(19) United States
(12) Patent Application Publication
(10) Pub. No.: US 2023/0200101 A1
(43) Pub. Date: Jun. 22, 2023

(54) LIGHT EMITTING ELEMENT AND AMINE COMPOUND FOR THE SAME

(71) Applicant: Samsung Display Co., Ltd., Yongin-si (KR)

(72) Inventors: Takuya UNO, Yokohama (JP); Takao SAKUMA, Yokohama (JP)

(21) Appl. No.: 18/084,382

(22) Filed: Dec. 19, 2022

(30) Foreign Application Priority Data
Dec. 21, 2021 (KR) 10-2021-0184235

(51) Int. Cl. H10K 50/15; C07D 405/14; H10K 85/60; C07D 409/14

(52) U.S. Cl. CPC H01L 51/5056; C07D 405/14; H01L 51/0072; H01L 51/0073; H01L 51/0061; C07D 409/14; H01L 51/0074; C07B 2200/05

(57) ABSTRACT

A light emitting element and an amine compound for the light emitting element are provided. The light emitting element includes a first electrode, a second electrode on the first electrode, and at least one functional layer between the first electrode and the second electrode. The functional layer includes the amine compound represented by a particular formula structure, and thus the luminous efficiency and service life of the light emitting element may be improved.

### CROSS-REFERENCE TO RELATED APPLICATION

This application claims priority to and the benefit of Korean Patent Application No. 10-2021-0184235, filed on Dec. 21, 2021, the entire content of which is hereby incorporated by reference.

### BACKGROUND

Aspects of one or more embodiments of the present disclosure relate to a light emitting element and an amine compound for the same, and for example, to a light emitting element including an amine compound in a hole transport region.

Recently, the development of an organic electroluminescence display device as an image display device is being actively conducted. The organic electroluminescence display device includes a self-luminescent light emitting element in which holes and electrons injected from a first electrode and a second electrode recombine in an emission layer, and thus a luminescent material of the emission layer emits light to implement display.

In the application of a light emitting element to a display device, there is a demand for a light emitting element having low driving voltage, high luminous efficiency, and a long service life, and development on materials for a light emitting element capable of stably attaining such characteristics is being continuously required (sought).

In some embodiments, development on materials of a hole transport region for suppressing the diffusion of exciton energy of the emission layer is being carried out in order to implement a highly efficient light emitting element.

### SUMMARY

An aspect of one or more embodiments of the present disclosure is directed toward a light emitting element exhibiting excellent or suitable luminous efficiency and long service life characteristics.

An aspect of one or more embodiments of the present disclosure is directed toward an amine compound which is a material for a light emitting element having high efficiency and long service life characteristics.

Additional aspects will be set forth in part in the description which follows and, in part, will be apparent from the description, or may be learned by practice of the presented embodiments of the disclosure.

An embodiment of the present disclosure provides an amine compound represented by Formula 1:

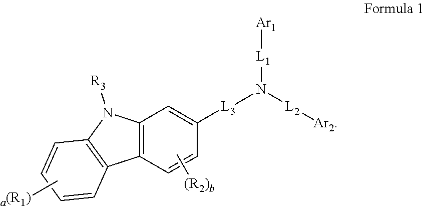

In Formula 1, R₁ and R₂ may each independently be a hydrogen atom, a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, and/or are bonded to adjacent group to form a ring, the embodiment in which R₁ and R₂ are each a substituted silyl group, a substituted alkyl group, a substituted alkenyl group, or a substituted aryl group, a substituent is not an amine group, R₃ is an unsubstituted aryl group having 6 to 10 ring-forming carbon atoms, or an aryl group having 6 to 10 ring-forming carbon atoms substituted with a deuterium atom, a halogen atom, or a substituted or unsubstituted alkyl group having 1 to 20 carbon atoms, a is an integer from 0 to 4, b is an integer from 0 to 3, at least one selected from among L₁ to L₃ is a direct linkage, and the L₁ to L₃ that are not a direct linkage are represented by any one selected from among Formula 2-1 to Formula 2-3, and Ar₁ and Ar₂ may each independently be represented by Formula 3:

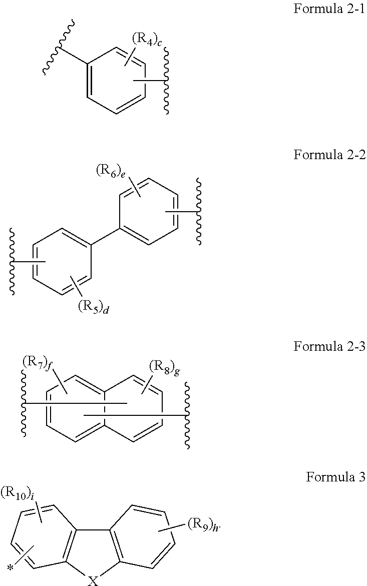

In Formula 2-1 to Formula 2-3, R₄ to R₈ may each independently be a hydrogen atom, a deuterium atom, a halogen atom, or a substituted or unsubstituted alkyl group having 1 to 20 carbon atoms, and c, d, e, f, and g may each independently be an integer from 0 to 4.

In Formula 3, X is O or S, R₉ and R₁₀ may each independently be a hydrogen atom, a deuterium atom, a halogen atom, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, and/or adjacent R₉s or adjacent R₁₀s are bonded to each other to form an aromatic hydrocarbon ring, and when adjacent R₉s or adjacent R₁₀s are bonded to each other to form an aromatic hydrocarbon ring, only one pair of R₉s or R₁₀s (either two R₉s or two R₁₀s) selected from among the plurality of R₉s and R₁₀s are bonded to each other to form an aromatic hydrocarbon ring, h is an integer from 0 to 4, i is an integer from 0 to 3, “-*” is a position linked to L₁ and L₂, and when L₁ and L₂ are each a direct linkage, the embodiment in which “-*” is linked to a nitrogen atom in the para relation to X is excluded.

In an embodiment, R₁ and R₂ may each independently be a hydrogen atom or a deuterium atom, and/or may be bonded to an adjacent group to form an aromatic hydrocarbon ring.

In an embodiment, R₃ may be represented by any one selected from among Substituent Group S1:

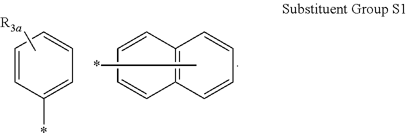

In Substituent Group S1, R₃ₐ is a hydrogen atom, a deuterium atom, an alkyl group having 1 to 10 carbon atoms, or a halogen atom.

In an embodiment, Formula 3 may be represented by Formula 3-1 or Formula 3-2:

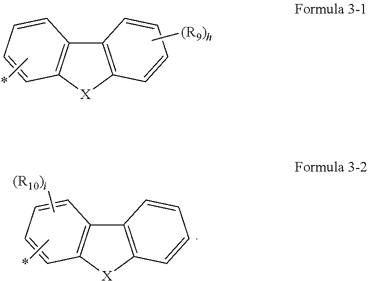

In Formula 3-1 and Formula 3-2, R₉, R₁₀, X, h, and i may each independently be the same as defined in Formula 3.

In some embodiments, in an embodiment, Formula 3 may be represented by any one selected from among 3-a to 3-k:

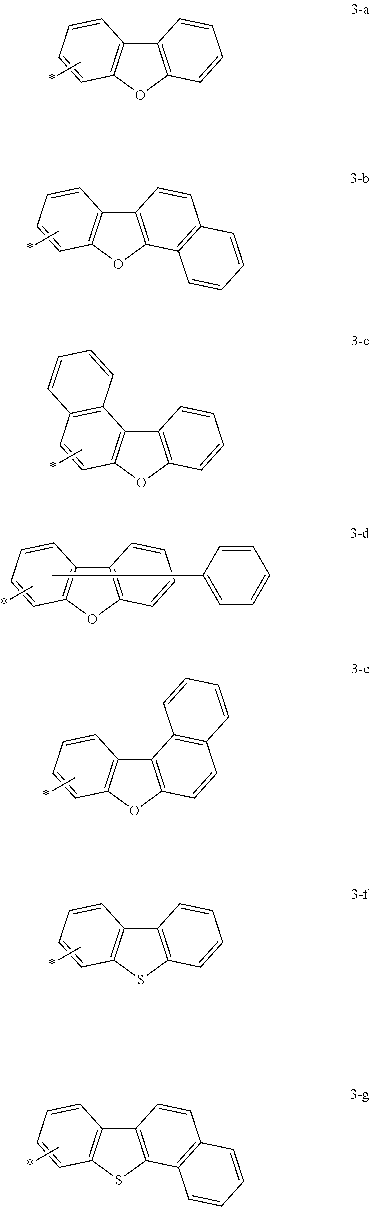

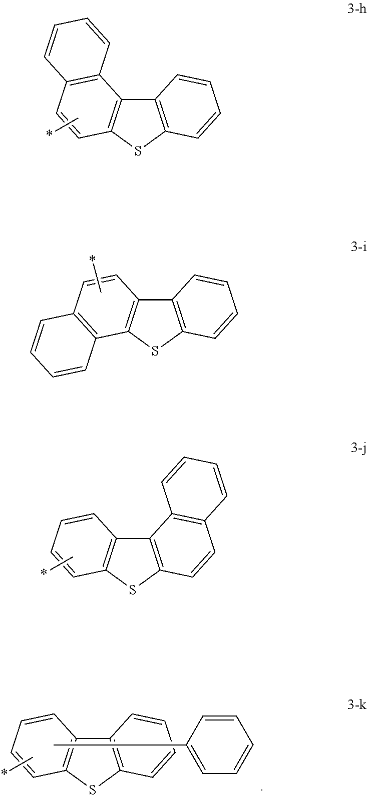

In an embodiment, at least one selected from among L₁ to L₃ may be a direct linkage, and the rest may be represented by any one selected from among Substituent Group S2:

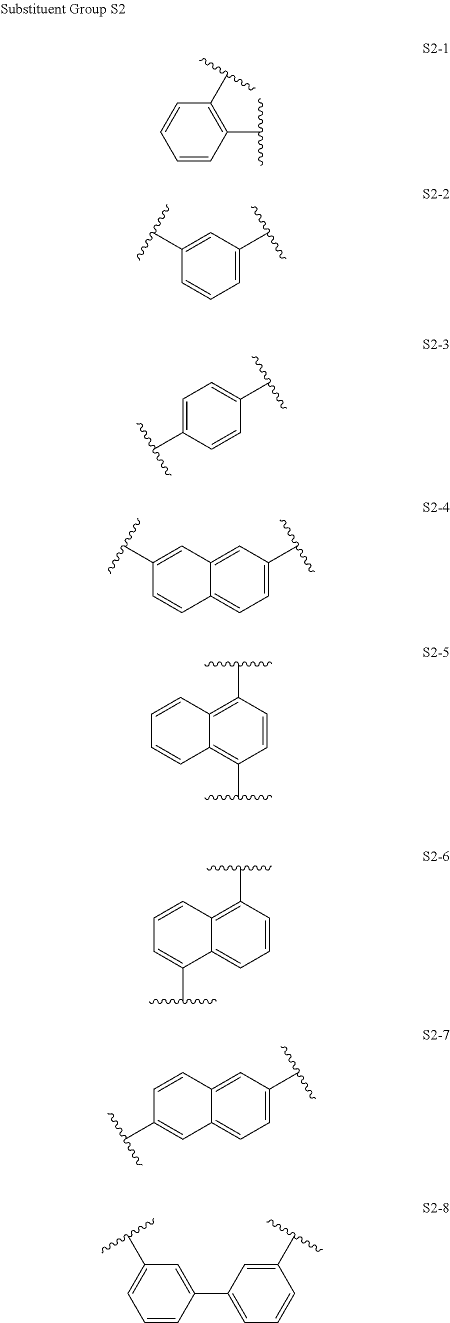

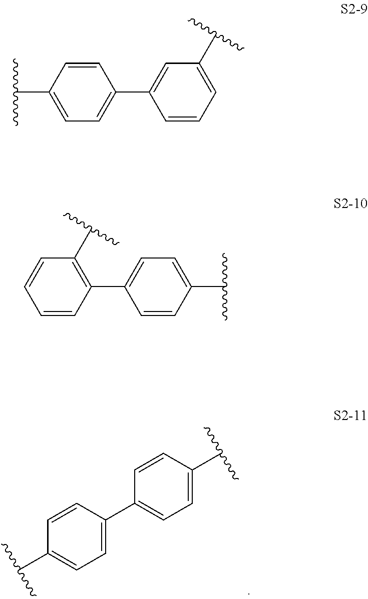 [SMILES: *c1ccc(-c2cccc(*)c2)cc1 ; *c1ccc(-c2ccccc2*)cc1 ; *c1ccc(-c2ccc(*)cc2)cc1]

In an embodiment, Formula 1 may be represented by Formula 4:

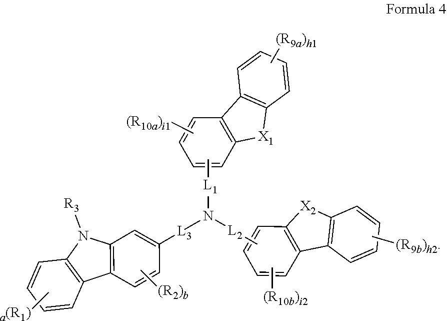

In Formula 4, X₁ and X₂ may each independently be O or S, R₉ₐ and R₁₀ₐ may each independently be a hydrogen atom, a deuterium atom, or a substituted or unsubstituted aryl group having 6 to 30 ring-forming carbon atoms, or adjacent R₉ₐ′ or adjacent R₁₀ₐs are bonded to each other to form an aromatic ring, R_{9b} and R_{10b} may each independently be a hydrogen atom, a deuterium atom, or a substituted or unsubstituted aryl group having 6 to 30 ring-forming carbon atoms, or adjacent R_{9b}s or adjacent R_{10b}s are bonded to each other to form an aromatic ring, h1 and h2 may each independently be an integer from 0 to 4, i1 and i2 may each independently be an integer from 0 to 3, and R₁ to R₃, a, b, and L₁ to L₃ may each independently be the same as defined in Formula 1.

In an embodiment, Formula 4 may be represented by any one selected from among Formula 4-1 to Formula 4-5:

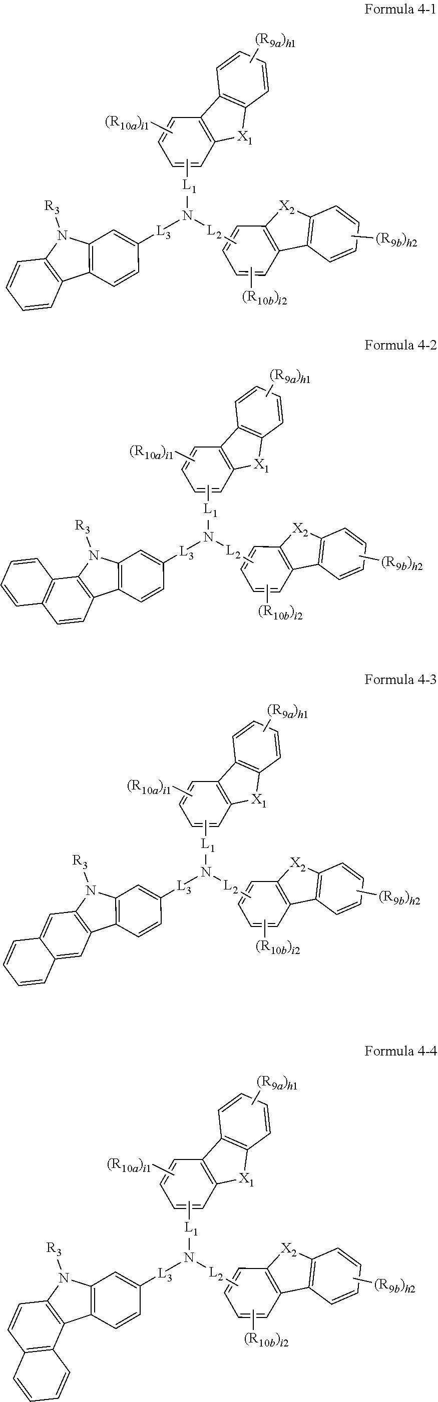

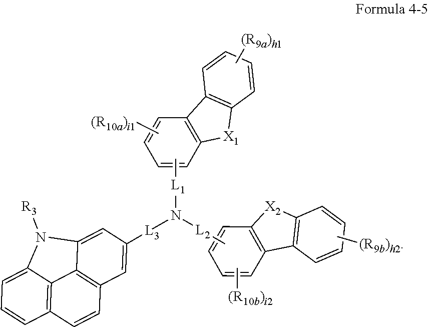

In Formula 4-1 to Formula 4-5, R₃, and L₁ to L₃ may each independently be the same as defined in Formula 1, and X₁, X₂, R₉ₐ, R_{9b}, R₁₀ₐ, R_{10b}, h1, h2, i1, and i2 may each independently be the same as defined in Formula 4.

In an embodiment of the present disclosure, a light emitting element includes: a first electrode; a second electrode on the first electrode; and at least one functional layer between the first electrode and the second electrode and includes the above-described amine compound.

In an embodiment, the at least one functional layer may include an emission layer, a hole transport region between the first electrode and the emission layer, and an electron transport region between the emission layer and the second electrode, and the hole transport region may include the amine compound.

In an embodiment, the hole transport region may include at least one of a hole injection layer, a hole transport layer, or an electron blocking layer, and at least one of the hole transport layer or the electron blocking layer may include the amine compound.

In an embodiment, the emission layer may include a compound represented by Formula E-1:

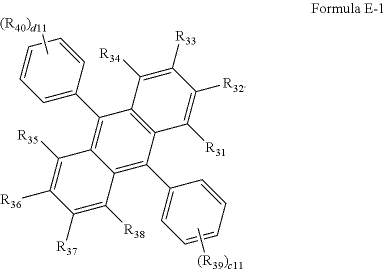

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or are bonded to an adjacent group to form a ring, and c11 and d11 may each independently be an integer from 0 to 5.

### BRIEF DESCRIPTION OF THE DRAWINGS

The accompanying drawings are included to provide a further understanding of of the present disclosure, and are incorporated in and constitute a part of this specification. The drawings illustrate example embodiments of the present disclosure and, together with the description, serve to explain principles of present disclosure. In the drawings:

FIG. 1 is a plan view illustrating a display device according to an embodiment;

FIG. 2 is a cross-sectional view of a display device according to an embodiment;

FIG. 3 is a cross-sectional view schematically illustrating a light emitting element according to an embodiment;

FIG. 4 is a cross-sectional view schematically illustrating a light emitting element according to an embodiment;

FIG. 5 is a cross-sectional view schematically illustrating a light emitting element according to an embodiment;

FIG. 6 is a cross-sectional view schematically illustrating a light emitting element according to an embodiment;

FIG. 7 is a cross-sectional view of a display device according to an embodiment;

FIG. 8 is a cross-sectional view of a display device according to an embodiment;

FIG. 9 is a cross-sectional view illustrating a display device according to an embodiment; and

FIG. 10 is a cross-sectional view illustrating a display device according to an embodiment.

### DETAILED DESCRIPTION

The present disclosure may be modified in many alternate forms, and thus specific embodiments will be exemplified in the drawings and described in more detail. It should be understood, however, that it is not intended to limit the present disclosure to the particular forms disclosed, but rather, is intended to cover all modifications, equivalents, and alternatives falling within the spirit and scope of the present disclosure.

When explaining each of drawings, like reference numerals are utilized for referring to like elements. In the accompanying drawings, the dimensions of each structure may be exaggeratingly illustrated for clarity of the present disclosure. It will be understood that, although the terms “first”, “second”, etc. may be utilized herein to describe one or more suitable components, these components should not be limited by these terms. These terms are only utilized to distinguish one component from another. For example, a first component could be termed a second component, and, similarly, a second component could be termed a first component, without departing from the scope of the present disclosure. As utilized herein, the singular forms, “a,” “an,” and “the” are intended to include the plural forms as well, unless the context clearly indicates otherwise.

In the present disclosure, it will be understood that the terms “include,” “have” and/or the like specify the presence of features, numbers, steps, operations, components, parts, or combinations thereof disclosed in the specification, but do not exclude the possibility of presence or addition of one or more other features, numbers, steps, operations, components, parts, or combinations thereof.

In the present disclosure, when a part such as a layer, a film, a region, or a plate is referred to as being “on” or “above” another part, it can be directly on the other part, or an intervening part may also be present. In contrast, when a part such as a layer, a film, a region, or a plate is referred to as being “under” or “below” another part, it can be directly under the other part, or an intervening part may also be present. In some embodiments, it will be understood that when a part is referred to as being “on” another part, it can be disposed above the other part, or disposed under the other part as well.

In the disclosure, the term “substituted or unsubstituted” may refer to substituted or unsubstituted with at least one substituent selected from the group including (e.g., consisting of) a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. In some embodiments, each of the substituents exemplified above may be substituted or unsubstituted. For example, a biphenyl group may be interpreted as an aryl group or a phenyl group substituted with a phenyl group.

In the disclosure, the phrase “bonded to an adjacent group to form a ring” may indicate that one is bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocyclic or polycyclic. In some embodiments, the rings formed by being bonded to each other may be connected to another ring to form a spiro structure.

In the disclosure, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. In some embodiments, two methyl groups in 4,5-dimethylphenanthrene may be interpreted as “adjacent groups” to each other.

In the disclosure, examples of the halogen atom may include a fluorine atom, a chlorine atom, a bromine atom, or an iodine atom.

In the disclosure, the alkyl group may be a linear, branched or cyclic type or kind. The number of carbons in the alkyl group may be 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a cyclopentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, a cyclohexyl group, a 4-methylcyclohexyl group, a 4-t-butylcyclohexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, a cyclooctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, etc., but the embodiment of the present disclosure is not limited thereto.

In the disclosure, an alkenyl group refers to a hydrocarbon group including at least one carbon double bond in the middle or terminal of an alkyl group having 2 or more carbon atoms. The alkenyl group may be a linear chain or a branched chain. The carbon number is not limited, but may be 2 to 30, 2 to 20 or 2 to 10. Examples of the alkenyl group include a vinyl group, a 1-butenyl group, a 1-pentenyl group, a 1,3-butadienyl aryl group, a styrenyl group, a styrylvinyl group, etc., without limitation.

In the disclosure, an alkynyl group refers to a hydrocarbon group including at least one carbon triple bond in the middle or terminal of an alkyl group having 2 or more carbon atoms. The alkynyl group may be a linear chain or a branched chain. The carbon number is not limited, but may be 2 to 30, 2 to 20 or 2 to 10. Examples of the alkynyl group include an ethynyl group, a propynyl group, etc., without limitation.

The hydrocarbon ring group herein refers to any functional group or substituent derived from an aliphatic hydrocarbon ring. The hydrocarbon ring group may be a saturated hydrocarbon ring group having 5 to 20 ring-forming carbon atoms.

In the disclosure, an aryl group refers to any functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, etc., but the embodiment of the present disclosure is not limited thereto.

In the disclosure, the fluorenyl group may be substituted, and two substituents may be bonded to each other to form a spiro structure. Examples of embodiments in which the fluorenyl group is substituted are as follows. However, the embodiment of the present disclosure is not limited thereto.

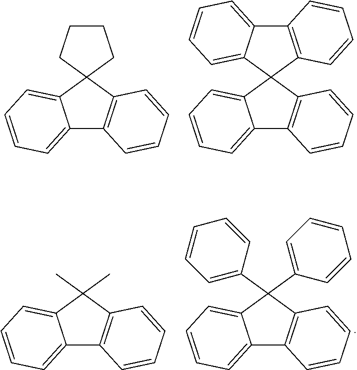

The heterocyclic group herein refers to any functional group or substituent derived from a ring including at least one of B, O, N, P, Si, or Se as a heteroatom.

The heterocyclic group includes an aliphatic heterocyclic group and an aromatic heterocyclic group. The aromatic heterocyclic group may be a heteroaryl group. The aliphatic heterocycle and the aromatic heterocycle may be monocyclic or polycyclic.

In the disclosure, the heterocyclic group may contain at least one of B, O, N, P, Si, or S as a heteroatom. When the heterocyclic group contains two or more heteroatoms, the two or more heteroatoms may be the same as or different from each other. In the disclosure, the heterocyclic group may be a monocyclic heterocyclic group or a polycyclic heterocyclic group, and is a concept including (e.g., may include) a heteroaryl group. The number of ring-forming carbon atoms in the heterocyclic group may be 2 to 30, 2 to 20, or 2 to 10.

In the disclosure, the aliphatic heterocyclic group may include one or more selected from among B, O, N, P, Si, and S as a heteroatom. The number of ring-forming carbon atoms in the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., but the embodiment of the present disclosure is not limited thereto.

The heteroaryl group herein may include at least one of B, O, N, P, Si, or S as a heteroatom. When the heteroaryl group contains two or more heteroatoms, the two or more heteroatoms may be the same as or different from each other. The heteroaryl group may be a monocyclic heteroaryl group or polycyclic heteroaryl group. The number of ring-forming carbon atoms in the heteroaryl group may be 2 to 30, 2 to 20, or 2 to 10. Examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, etc., but the embodiment of the present disclosure is not limited thereto.

In the disclosure, the above description of the aryl group may be applied to an arylene group except that the arylene group is a divalent group. The above description of the heteroaryl group may be applied to a heteroarylene group except that the heteroarylene group is a divalent group.

In the disclosure, the boryl group includes an alkyl boryl group and an aryl boryl group. Examples of the boryl group may include a dimethylboryl group, a diethylboryl group, a t-butylmethylboryl group, a diphenylboryl group, a phenylboryl group, etc., but the embodiment of the present disclosure is not limited thereto. For example, the alkyl group in the alkyl boryl group is the same as the examples of the alkyl group described above, and the aryl group in the aryl boryl group is the same as the examples of the aryl group described above.

In the disclosure, a silyl group includes an alkyl silyl group and/or an aryl silyl group. Examples of the silyl group may include trimethylsilyl, triethylsilyl, t-butyldimethylsilyl, vinyldimethylsilyl, propyldimethylsilyl, triphenylsilyl, diphenylsilyl, phenylsilyl, etc. However, an embodiment of the present disclosure is not limited thereto.

In the disclosure, the number of ring-forming carbon atoms in the carbonyl group is not limited, but may be 1 to 40, 1 to 30, or 1 to 20. For example, the carbonyl group may have the following structures, but the embodiment of the present disclosure is not limited thereto.

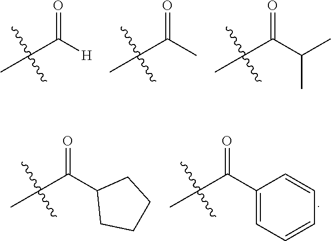

In the disclosure, the number of carbon atoms in the sulfinyl group and the sulfonyl group is not limited, but may be 1 to 30. The sulfinyl group may include an alkyl sulfinyl group and an aryl sulfinyl group. The sulfonyl group may include an alkyl sulfonyl group and/or an aryl sulfonyl group.

In the disclosure, a thio group may include an alkylthio group and/or an arylthio group. The thio group may refer to a sulfur atom that is bonded to the alkyl group or the aryl group as defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, etc., but the embodiment of the present disclosure is not limited thereto.

In the disclosure, an oxy group may refer to an oxygen atom that is bonded to the alkyl group or the aryl group as defined above. The oxy group may include an alkoxy group and/or an aryl oxy group. The alkoxy group may be a linear chain, a branched chain or a ring chain. The number of carbon atoms in the alkoxy group is not specifically limited, but may be, for example, 1 to 20 or 1 to 10. Examples of the oxy group include methoxy, ethoxy, n-propoxy, isopropoxy, butoxy, pentyloxy, hexyloxy, octyloxy, nonyloxy, decyloxy, benzyloxy, etc., but the embodiment of the present disclosure is not limited thereto.

In the disclosure, the number of carbon atoms in an amine group is not limited, but may be 1 to 30. The amine group may include an alkyl amine group and an aryl amine group. Examples of the amine group include a methylamine group, a dimethylamine group, a phenylamine group, a diphenylamine group, a naphthylamine group, a 9-methyl-anthracenylamine group, etc., but the embodiment of the present disclosure is not limited thereto.

In the disclosure, the alkyl group selected from among an alkylthio group, an alkylsulfoxy group, an alkylaryl group, an alkylamino group, an alkyl boron group, an alkyl silyl group, and an alkyl amine group is the same as the examples of the alkyl group described above.

In the disclosure, the aryl group selected from among an aryloxy group, an arylthio group, an arylsulfoxy group, an arylamino group, an arylboron group, an arylsilyl group, an arylamine group is the same as the examples of the aryl group described above.

In the disclosure, a direct linkage may refer to a single bond.

In some embodiments,

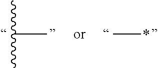

herein refers to a position to be connected.

Hereinafter, embodiments of the present disclosure will be described with reference to the accompanying drawings.

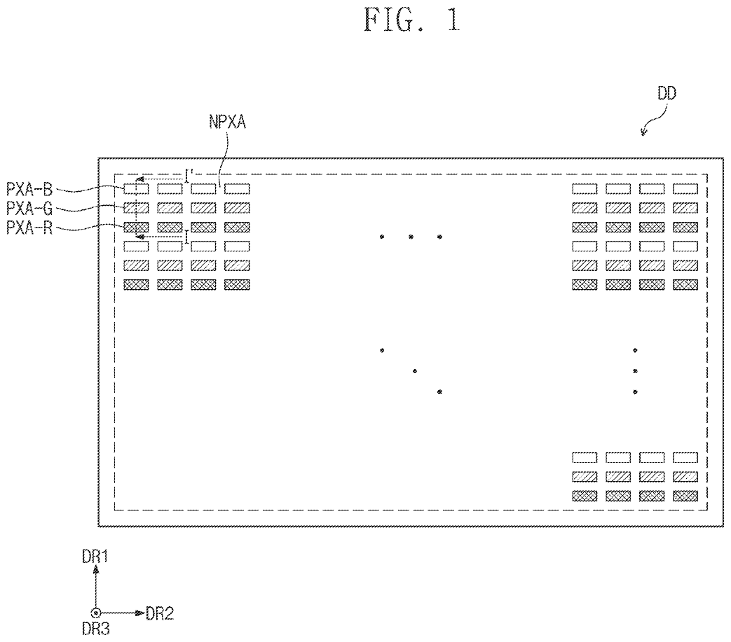

FIG. 1 is a plan view illustrating an embodiment of a display device DD.

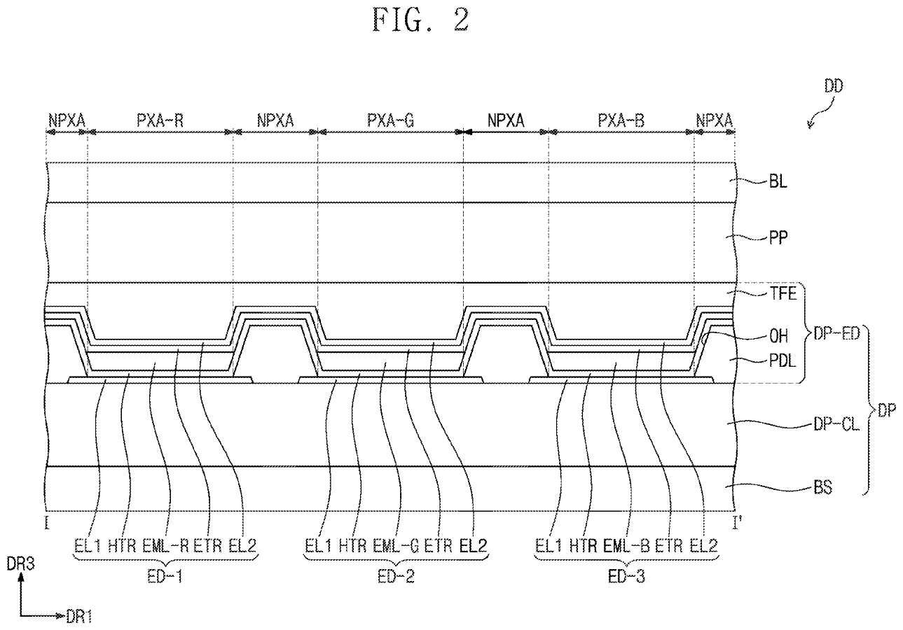

FIG. 2 is a cross-sectional view of the display device DD of the embodiment. FIG. 2 is a cross-sectional view illustrating a part taken along line I-I′ of FIG. 1.

The display device DD may include a display panel DP and an optical layer PP on the display panel DP. The display panel DP includes light emitting elements ED-1, ED-2, and ED-3. The display device DD may include a plurality of light emitting elements ED-1, ED-2, and ED-3. The optical layer PP may be on the display panel DP to control reflected light in the display panel DP due to external light. The optical layer PP may include, for example, a polarization layer or a color filter layer. In some embodiments, the optical layer PP may not be provided in the display device DD of an embodiment.

A base substrate BL may be on the optical layer PP. The base substrate BL may be a member which provides a base surface on which the optical layer PP disposed. The base substrate BL may be a glass substrate, a metal substrate, a plastic substrate, etc. However, the embodiment of the present disclosure is not limited thereto, and the base substrate BL may be an inorganic layer, an organic layer, or a composite material layer. In some embodiments, the base substrate BL may not be provided.

In an embodiment, the circuit layer DP-CL is on the base layer BS, and the circuit layer DP-CL may include a plurality of transistors. Each of the transistors may include a control electrode, an input electrode, and an output electrode. For example, the circuit layer DP-CL may include a switching transistor and a driving transistor for driving the light emitting elements ED-1, ED-2, and ED-3 of the display element layer DP-ED.

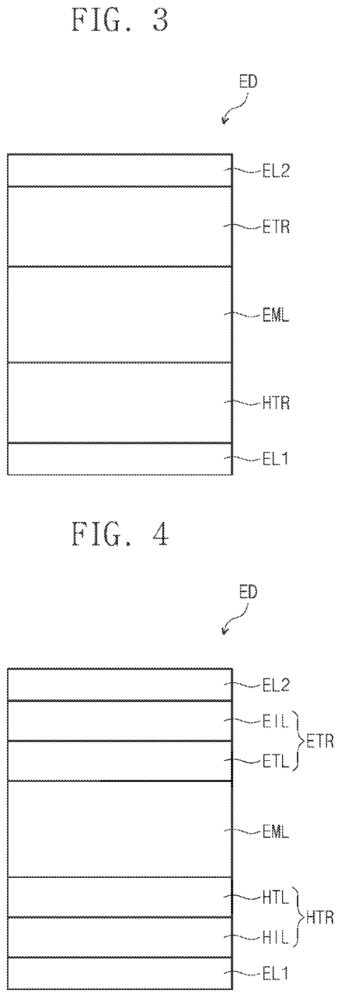

Each of the light emitting elements ED-1, ED-2, and ED-3 may have a structure of a light emitting element ED of an embodiment according to FIGS. 3 to 6, which will be described in more detail. Each of the light emitting elements ED-1, ED-2 and ED-3 may include a first electrode EL1, a hole transport region HTR, emission layers EML-R, EML-G and EML-B, an electron transport region ETR, and a second electrode EL2.

FIG. 2 illustrates an embodiment in which the emission layers EML-R, EML-G, and EML-B of the light emitting elements ED-1, ED-2, and ED-3 are in openings OH defined in the pixel defining film PDL, and the hole transport region HTR, the electron transport region ETR, and the second electrode EL2 are provided as a common layer in the entire light emitting elements ED-1, ED-2, and ED-3. However, the embodiment of the present disclosure is not limited thereto, and the hole transport region HTR and the electron transport region ETR in an embodiment may be provided by being patterned inside the openings OH defined in the pixel defining film PDL. For example, the hole transport region HTR, the emission layers EML-R, EML-G, and EML-B, and the electron transport region ETR of the light emitting elements ED-1, ED-2, and ED-3 in an embodiment may be provided by being patterned in an inkjet printing method.

The encapsulation layer TFE may cover the light emitting elements ED-1, ED-2 and ED-3. The encapsulation layer TFE may seal the display element layer DP-ED. The encapsulation layer TFE may be a thin film encapsulation layer. The encapsulation layer TFE may be formed by laminating one layer or a plurality of layers. The encapsulation layer TFE includes at least one insulation layer. The encapsulation layer TFE according to an embodiment may include at least one inorganic film (hereinafter, an encapsulation-inorganic film). The encapsulation layer TFE according to an embodiment may also include at least one organic film (hereinafter, an encapsulation-organic film) and at least one encapsulation-inorganic film.

The encapsulation-inorganic film protects (reduces) the display element layer DP-ED from moisture/oxygen, and the encapsulation-organic film protects (reduces) the display element layer DP-ED from foreign substances such as dust particles. The encapsulation-inorganic film may include silicon nitride, silicon oxynitride, silicon oxide, titanium oxide, aluminum oxide, and/or the like, but the embodiment of the present disclosure is not limited thereto. The encapsulation-organic film may include an acrylic-based compound, an epoxy-based compound, and/or the like. The encapsulation-organic film may include a photopolymerizable organic material, but the embodiment of the present disclosure is not limited thereto.

The encapsulation layer TFE may be on the second electrode EL2 and may be disposed filling the opening OH.

Referring to FIGS. 1 and 2, the display device DD may include a non-light emitting region NPXA and light emitting regions PXA-R, PXA-G and PXA-B. The light emitting regions PXA-R, PXA-G and PXA-B may be regions in which light generated by the respective light emitting elements ED-1, ED-2 and ED-3 is emitted. The light emitting regions PXA-R, PXA-G, and PXA-B may be spaced apart from each other on a plane (e.g., in a plan view).

Each of the light emitting regions PXA-R, PXA-G, and PXA-B may be a region divided by the pixel defining film PDL. The non-light emitting regions NPXA may be regions between the adjacent light emitting regions PXA-R, PXA-G, and PXA-B, which correspond to portions of the pixel defining film PDL. In some embodiments, in the disclosure, the light emitting regions PXA-R, PXA-G, and PXA-B may respectively correspond to pixels. The pixel defining film PDL may divide the light emitting elements ED-1, ED-2, and ED-3. The emission layers EML-R, EML-G and EML-B of the light emitting elements ED-1, ED-2 and ED-3 may be disposed in openings OH defined in the pixel defining film PDL and separated from each other.

The light emitting regions PXA-R, PXA-G and PXA-B may be divided into a plurality of groups according to the color of light generated from the light emitting elements ED-1, ED-2 and ED-3. In the display device DD of an embodiment shown in FIGS. 1 and 2, three light emitting regions PXA-R, PXA-G, and PXA-B which emit red light, green light, and blue light, respectively are illustrated. For example, the display device DD of an embodiment may include the red light emitting region PXA-R, the green light emitting region PXA-G, and the blue light emitting region PXA-B that are separated from each other.

In the display device DD according to an embodiment, the plurality of light emitting elements ED-1, ED-2 and ED-3 may emit light beams having wavelengths different from each other. For example, in an embodiment, the display device DD may include a first light emitting element ED-1 that emits red light, a second light emitting element ED-2 that emits green light, and a third light emitting element ED-3 that emits blue light. For example, the red light emitting region PXA-R, the green light emitting region PXA-G, and the blue light emitting region PXA-B of the display device DD may correspond to the first light emitting element ED-1, the second light emitting element ED-2, and the third light emitting element ED-3, respectively.

However, the embodiment of the present disclosure is not limited thereto, and the first to third light emitting elements ED-1, ED-2, and ED-3 may emit light beams in substantially the same wavelength range or at least one light emitting element may emit a light beam in a wavelength range different from the others. For example, the first to third light emitting elements ED-1, ED-2, and ED-3 may all emit blue light.

The light emitting regions PXA-R, PXA-G, and PXA-B in the display device DD according to an embodiment may be arranged in a stripe form. Referring to FIG. 1, the plurality of red light emitting regions PXA-R may be arranged with each other along a second direction axis DR2, the plurality of green light emitting regions PXA-G may be arranged with each other along the second direction axis DR2, and the plurality of blue light emitting regions PXA-B may be arranged with each other along the second direction axis DR2. In some embodiments, the red light emitting region PXA-R, the green light emitting region PXA-G, and the blue light emitting region PXA-B may be alternately arranged in this order along a first direction axis DR1. (DR3 is a third direction which is normal or perpendicular to the plane defined by the first direction DR1 and the second direction DR2).

FIGS. 1 and 2 illustrate that all the light emitting regions PXA-R, PXA-G, and PXA-B have a substantially similar area, but the embodiment of the present disclosure is not limited thereto. Thus, the light emitting regions PXA-R, PXA-G, and PXA-B may have different areas from each other according to the wavelength range of the emitted light. In this embodiment, the areas of the light emitting regions PXA-R, PXA-G, and PXA-B may refer to areas when viewed on a plane defined by the first direction axis DR1 and the second direction axis DR2 (e.g., when viewed in a plan view).

In some embodiments, an arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in one or more suitable combinations according to the characteristics of display quality required in the display device DD. For example, the arrangement form of the light emitting regions PXA-R, PXA-G, and PXA-B may be a PENTILE® arrangement form (for example, an RGBG matrix, an RGBG structure, or RGBG matrix structure) or a Diamond Pixel™ arrangement form (e.g., a display (e.g., an OLED display) containing red, blue, and green (RGB) light emitting regions arranged in the shape of diamonds. PENTILE® is a duly registered trademark of Samsung Display Co., Ltd. Diamond Pixel™ is a trademark of Samsung's displays.

In some embodiments, the areas of the light emitting regions PXA-R, PXA-G, and PXA-B may be different from each other. For example, in an embodiment, the area of the green light emitting region PXA-G may be smaller than that of the blue light emitting region PXA-B, but the embodiment of the present disclosure is not limited thereto.

Hereinafter, FIGS. 3 to 6 are cross-sectional views schematically illustrating light emitting elements ED according to embodiments. The light emitting elements ED according to embodiments each may include a first electrode EL1, a second electrode EL2 facing the first electrode EL1, and at least one functional layer between the first electrode EL1 and the second electrode EL2. Each of the light emitting elements ED of embodiments may include an amine compound of an embodiment, which will be described in more detail, in at least one functional layer.

Each of the light emitting elements ED may include, as at least one functional layer, a hole transport region HTR, an emission layer EML, and an electron transport region ETR that are sequentially stacked. Referring to FIG. 3, the light emitting element ED of an embodiment may include a first electrode EL1, a hole transport region HTR, an emission layer EML, an electron transport region ETR, and a second electrode EL2 that are sequentially stacked.

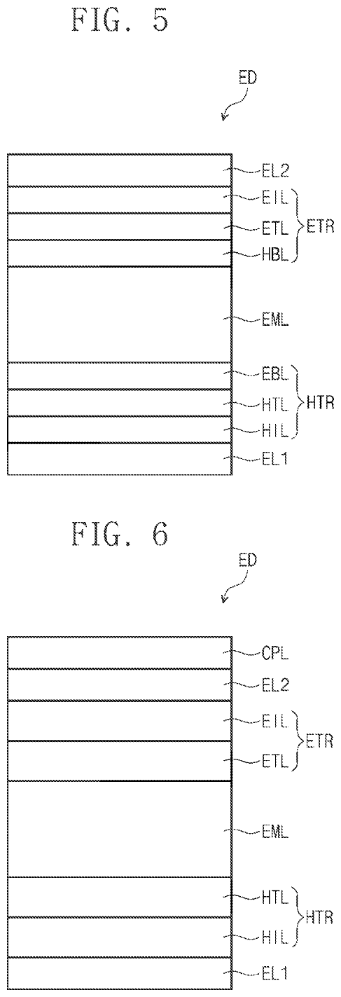

Compared with FIG. 3, FIG. 4 illustrates a cross-sectional view of a light emitting element ED of an embodiment, in which a hole transport region HTR includes a hole injection layer HIL and a hole transport layer HTL, and an electron transport region ETR includes an electron injection layer EIL and an electron transport layer ETL. In some embodiments, compared with FIG. 3, FIG. 5 illustrates a cross-sectional view of a light emitting element ED of an embodiment, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared with FIG. 4, FIG. 6 illustrates a cross-sectional view of a light emitting element ED of an embodiment including a capping layer CPL on a second electrode EL2.

The light emitting element ED of an embodiment may include an amine compound of an embodiment, which will be described in more detail, in the hole transport region HTR. In the light emitting element ED of an embodiment, at least one of a hole injection layer HIL, a hole transport layer HTL, or an electron blocking layer EBL in the hole transport region HTR may include the amine compound of an embodiment. For example, in the light emitting element ED of an embodiment, at least one of the hole transport layer HTL or the electron blocking layer EBL may include the amine compound of an embodiment.

In the light emitting element ED according to an embodiment, the first electrode EL1 has conductivity (e.g., is a conductor). The first electrode EL1 may be formed of a metal material, a metal alloy, or a conductive compound. The first electrode EL1 may be an anode or a cathode. However, the embodiment of the present disclosure is not limited thereto. In some embodiments, the first electrode EL1 may be a pixel electrode. The first electrode EL1 may be a transmissive electrode, a transflective electrode, or a reflective electrode. The first electrode EL1 may include at least one selected from among Ag, Mg, Cu, Al, Pt, Pd, Au, Ni, Nd, Ir, Cr, Li, Ca, LiF, Mo, Ti, W, In, Sn, Zn, compounds comprising one or more of the foregoing elements, combinations of two or more of the foregoing elements or compounds, mixtures of two or or more of the foregoing elements or compounds, and/or oxides thereof.

When the first electrode EL1 is the transmissive electrode, the first electrode EL1 may include a transparent metal oxide such as indium tin oxide (ITO), indium zinc oxide (IZO), zinc oxide (ZnO), and/or indium tin zinc oxide (ITZO). When the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, and/or a compound or mixture thereof (e.g., a mixture of Ag and Mg). In some embodiments, the first electrode EL1 may have a multilayer structure including a reflective film or a transflective film formed of the above-described materials, and a transparent conductive film formed of ITO, IZO, ZnO, ITZO, etc. For example, the first electrode EL1 may have a three-layer structure of ITO/Ag/ITO, but the embodiment of the present disclosure is not limited thereto. The embodiment of the present disclosure is not limited thereto, and the first electrode EL1 may include the above-described metal materials, combinations of at least two metal materials of the above-described metal materials, oxides of the above-described metal materials, and/or the like. The thickness of the first electrode EL1 may be from about 700 Å to about 10,000 Å. For example, the thickness of the first electrode EL1 may be from about 1,000 Å to about 3,000 Å.

The hole transport region HTR may be provided on the first electrode EL1. The hole transport region HTR may have a single layer formed of a single material, a single layer formed of a plurality of different materials, or a multilayer structure including a plurality of layers formed of a plurality of different materials.

The hole transport region HTR may include at least one of the hole injection layer HIL, the hole transport layer HTL, or the electron blocking layer EBL. In some embodiments, the hole transport region HTR may include a plurality of stacked hole transport layers HTR.

In some embodiments, the hole transport region HTR may have a single layer structure of the hole injection layer HIL or the hole transport layer HTL, or may have a single layer structure formed of a hole injection material and a hole transport material. In an embodiment, the hole transport region HTR may have a single layer structure formed of a plurality of different materials, or a structure in which a hole injection layer HIL/hole transport layer HTL, a hole injection layer HIL/hole transport layer HTL/buffer layer, a hole injection layer HIL/buffer layer, or a hole transport layer HTL/buffer layer are stacked in order from the first electrode EL1, but the embodiment of the present disclosure is not limited thereto.

The thickness of the hole transport region HTR may be, for example, from about 50 Å to about 15,000 Å. The hole transport region HTR may be formed utilizing one or more suitable methods such as a vacuum deposition method, a spin coating method, a cast method, a Langmuir-Blodgett (LB) method, an inkjet printing method, a laser printing method, and/or a laser induced thermal imaging (LITI) method.

The light emitting element ED of an embodiment may include the amine compound of an embodiment in the hole transport region HTR. In the light emitting element ED of an embodiment, the hole transport layer HTL or the electron blocking layer EBL in the hole transport region HTR may include the amine compound of an embodiment.

In an embodiment according to the present disclosure, the amine compound may be represented by Formula 1. Any hydrogen atom included in the amine compound represented by Formula 1 of an embodiment may be substituted with a deuterium atom. For example, the amine compound of an embodiment may contain a deuterium atom or a substituent including a deuterium atom. For example, the amine compound of an embodiment may include at least one deuterium atom as a substituent.

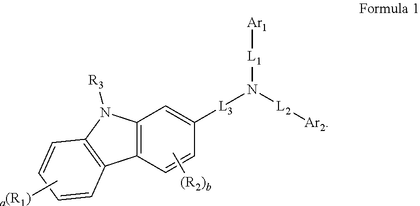

In Formula 1, R₁ and R₂ may each independently be a hydrogen atom, a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, and/or may be bonded to an adjacent group to form a ring. Here, when R₁ and R₂ are each a substituted silyl group, a substituted alkyl group, a substituted alkenyl group, or a substituted aryl group, the embodiment in which the substituent is an amine group may be excluded.

When R₁ and R₂ are bonded to an adjacent group to form a ring, the embodiment in which a ring containing a heteroatom of O, S, or N is formed may be excluded. For example, R₁ and R₂ may be bonded to an adjacent group to form an aromatic hydrocarbon ring. For example, adjacent R₁'s may be bonded to each other to form an aromatic hydrocarbon ring, or R₁ and R₂, which are adjacent, may be bonded to form an aromatic hydrocarbon ring. In some embodiments, adjacent R₂s may be bonded to each other to form an aromatic hydrocarbon ring.

In an embodiment, R₁ and R₂ may each independently be a hydrogen atom or a deuterium atom, and/or may be bonded to an adjacent group to form an aromatic ring. When R₁ and R₂ are bonded to an adjacent group to form an aromatic hydrocarbon ring, adjacent two R₁s may be bonded to each other to form an aromatic hydrocarbon ring, or R₁ and R₂, which are adjacent, may be bonded to form an aromatic hydrocarbon ring.

In Formula 1, a may be an integer from 0 to 4, and b may be an integer from 0 to 3. When a is an integer of 2 or greater, a plurality R₁s may all be the same or at least one may be different from the rest. When b is an integer of 2 or greater, a plurality of R₂s may all be the same or at least one may be different from the rest. The embodiment in which a is 0 may be the same as the embodiment in which a is 4 and R₁s are hydrogen atoms. The embodiment in which b is 0 may be the same as the embodiment in which b is 3 and R₂s are hydrogen atoms. In some embodiments, when each of a and b is 0, the benzene ring moieties in the carbazole moiety included in the amine compound of an embodiment may be unsubstituted.

In Formula 1, R₃ may be a substituted or unsubstituted aryl group having 6 to 10 ring-forming carbon atoms. When R₃ is a substituted aryl group, the substituent may exclude an amine group. For example, R₃ may be an unsubstituted aryl group, or an aryl group substituted with a deuterium atom, a halogen atom, or a substituted or unsubstituted alkyl group having 1 to 20 carbon atoms. In some embodiments, when R₃ is an aryl group substituted with a halogen atom, R₃ may include a fluorine atom (F) as a heteroatom.

In an embodiment, R₃ may be represented by any one selected from among Substituent Group S1. In Substituent Group S1, “*-” is bonded to the nitrogen atom (N) of the carbazole moiety included in the amine compound of an embodiment.

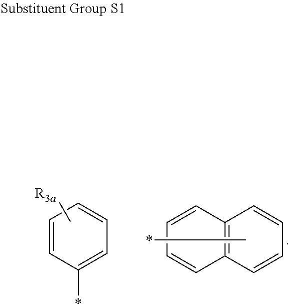

In Substituent Group S1, R₃ₐ may be a hydrogen atom, a deuterium atom, an alkyl group having 1 to 10 carbon atoms, or a halogen atom. For example, R₃ₐ may be a hydrogen atom, a deuterium atom, a t-butyl group, or a fluorine atom (F).

At least one selected from among L₁ to L₃ is a direct linkage, and the L₁ to L₃ that are not a direct linkage are represented by any one selected from among Formula 2-1 to Formula 2-3:

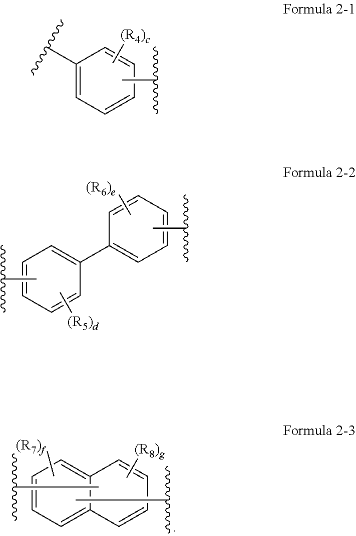

In Formula 2-1 to Formula 2-3, R₄ to R₈ may each independently be a hydrogen atom, a deuterium atom, a halogen atom, or a substituted or unsubstituted alkyl group having 1 to 20 carbon atoms. For example, R₄ to R₈ may each independently be a hydrogen atom or a deuterium atom, but the embodiment of the present disclosure is not limited thereto.

In Formula 2-1 to Formula 2-3, c, d, e, f, and g may each independently be an integer from 0 to 4. When each of c, d, e, f, and g is an integer of 2 or greater, a plurality of R₄s to R₈s may each be the same or one or more may be different. For example, when c is 2, two R₄s may be the same as or different from each other. In some embodiments, such a description may be equally applied to R₅, R₆, R₇, and R₈. In some embodiments, not both (e.g., simultaneously) f and g are 4 at the same time. For example, depending on the position at which Formula 2-3 is linked to Formula 1, both (e.g., simultaneously) f and g may be 3, or any one selected from among f and g may be 4, and the other (the one not selected) may be 2.

For example, the embodiment in which c is 0 may be the same as the embodiment in which c is 4 and R₄s are hydrogen atoms, the embodiment in which d is 0 may be the same as the embodiment in which d is 4 and R₅s are hydrogen atoms, the embodiment in which e is 0 may be the same as the embodiment in which e is 4 and R₆s are hydrogen atoms, the embodiment in which f is 0 may be the same as the embodiment in which f is 4 and R₇s are hydrogen atoms, and the embodiment in which g is 0 may be the same as the embodiment in which g is 4 and R₈′ are hydrogen atoms. In some embodiments, when each of c, d, e, f, and g is 0, L₁ to L₃ may be unsubstituted.

In Formula 2-1 to Formula 2-3,

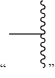

is a part linked to the amine compound.

For example, any one selected from among two

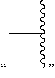

is a part linked to the nitrogen atom (N) of the amine compound, and the other (the one not selected) is a part linked to the carbazole moiety, Ar₁, or Ar₂ of the amine compound.

In an embodiment, at least one selected from among L₁ to L₃ may be a direct linkage, and the rest may be represented by any one selected from among Substituent Group S2. However, the embodiment of the present disclosure is not limited thereto.

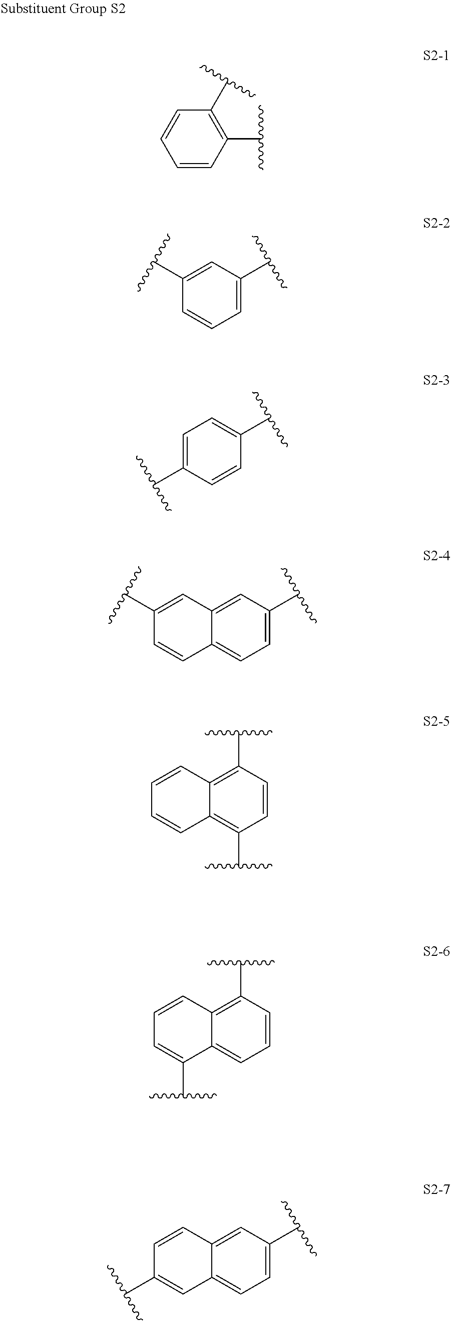

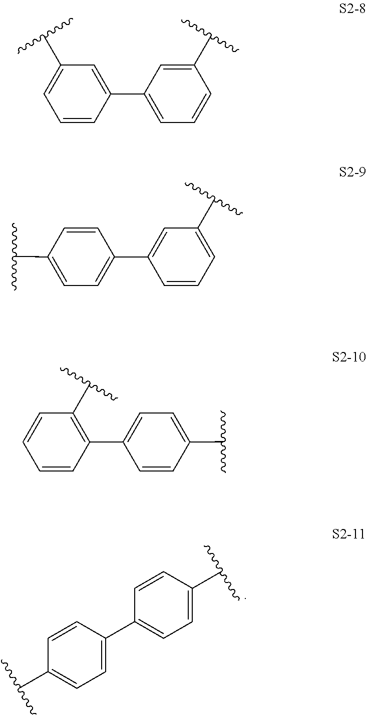 [SMILES: *c1cccc(-c2cccc(*)c2)c1 ; *c1ccc(-c2cccc(*)c2)cc1 ; *c1ccc(-c2ccccc2*)cc1 ; *c1ccc(-c2ccc(*)cc2)cc1]

In Formula 1, Ar₁ and Ar₂ may each independently be a substituted or unsubstituted dibenzoheterole group. In an embodiment, Ar₁ and Ar₂ may each independently be represented by Formula 3:

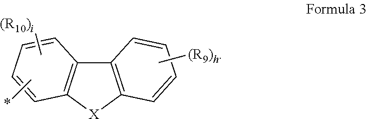

In Formula 3, X may be an oxygen atom (O) or a sulfur atom (S). For example, when X is an oxygen atom (O), Ar₁ and Ar₂ may include a dibenzofuran skeleton, and when X is a sulfur atom (S), Ar₁ and Ar₂ may include a dibenzothiophene skeleton.

In Formula 3, R₉ and R₁₀ may each independently be a hydrogen atom, a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, or adjacent R₉s or adjacent R₁₀s may be bonded to each other to form an aromatic hydrocarbon ring. For example, R₉ and R₁₀ may each independently be a hydrogen atom, a deuterium atom, or a substituted or unsubstituted phenyl group, and/or adjacent R₉s or adjacent R₁₀s may be bonded to each other to form an aromatic hydrocarbon ring.

In an embodiment, when adjacent R₉s or adjacent R₁₀s are bonded to each other to form an aromatic hydrocarbon ring, only one pair of R₉s or R₁₀s (either two R₉s or two R₁₀s) selected from among a plurality of R₉'s and R₁₀'s may be bonded to each other to form an aromatic hydrocarbon ring. For example, Ar₁ and Ar₂ may each independently be that one heteroaromatic hydrocarbon ring that is condensed at the benzene ring of the dibenzoheterole moiety. For example, Ar₁ and Ar₂ represented by Formula 3 may be two adjacent R₉s that are bonded to each other to form an aromatic hydrocarbon ring, or two adjacent R₁₀s that are bonded to each other to form an aromatic hydrocarbon ring. When adjacent R₉s or adjacent R₁₀s are bonded to each other to form an aromatic hydrocarbon ring, Ar₁ and Ar₂ may include a benzonaphthofuran skeleton or a benzonaphthothiophene skeleton.

In Formula 3, h may be an integer from 0 to 4, and i may be an integer from 0 to 3. When h is an integer of 2 or greater, a plurality of R₉s may all be the same or one or more may be different. When i is an integer of 2 or greater, a plurality R₁₀s may all be the same or one or more may be different. The embodiment in which h is 0 may be the same as the embodiment in which h is 4 and R₉s are hydrogen atoms. The embodiment in which i is 0 may be the same as the embodiment in which i is 3 and R₁₀s are hydrogen atoms. In some embodiments, when each of h and i is 0, Ar₁ and Ar₂ may be unsubstituted.

In Formula 3, “-*” is a position linked to L₁ or L₂. For example, “-*” is a position in which Ar₁ and Ar₂ are linked to L₁ and L₂, respectively. When L₁ and L₂ are each a direct linkage, “-*” corresponds to a position linked to the nitrogen atom (N) of the amine compound. In an embodiment, when L₁ and L₂ are each a direct linkage, the embodiment in which “-*” is linked to the nitrogen atom of the amine compound of an embodiment in the para relation to X may be excluded. For example, referring to Formula 3a, the amine compound of an embodiment may not be directly bonded to the nitrogen atom at the second position of the dibenzoheterole group, but may be directly bonded to the nitrogen atom at the first position, third position, or fourth position of the dibenzoheterole group.

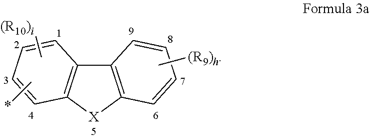

In the amine compound of an embodiment, Formula 3 may be represented by Formula 3-1 or Formula 3-2. Formula 3-1 and Formula 3-2 correspond to the structures in which R₉ and R₁₀, respectively, are substituents in Formula 3. In Formula 3-1 and Formula 3-2, the same as defined in Formula 3 may be applied to X, R₉, R₁₀, h, and i. For example, in Formula 3-1, X may be O or S. R₉ may be a hydrogen atom, a deuterium atom, or a substituted or unsubstituted aryl group having 6 to 15 ring-forming carbon atoms, and/or a pair of adjacent R₉s may be bonded to each other to form an aromatic hydrocarbon ring. h may be an integer from 0 to 4. In Formula 3-2, X may be O or S. R₁₀ may be a hydrogen atom, a deuterium atom, or a substituted or unsubstituted aryl group having 6 to 15 ring-forming carbon atoms, and/or a pair of adjacent R₁₀s may be bonded to each other to form an aromatic hydrocarbon ring. i may be an integer from 0 to 3.

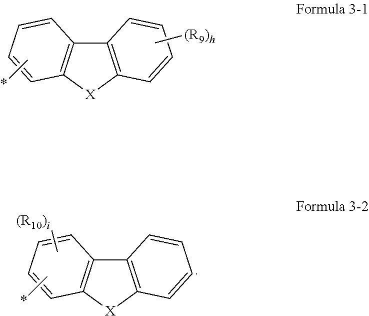

In an embodiment, Formula 3 may be represented by any one selected from among 3-a to 3-k. In 3-a to 3-k, “-*” may be a position linked to L₁ or L₂. When L₁ and L₂ are each a direct linkage, “-*” may be a position linked to the nitrogen atom (N) of the amine compound.

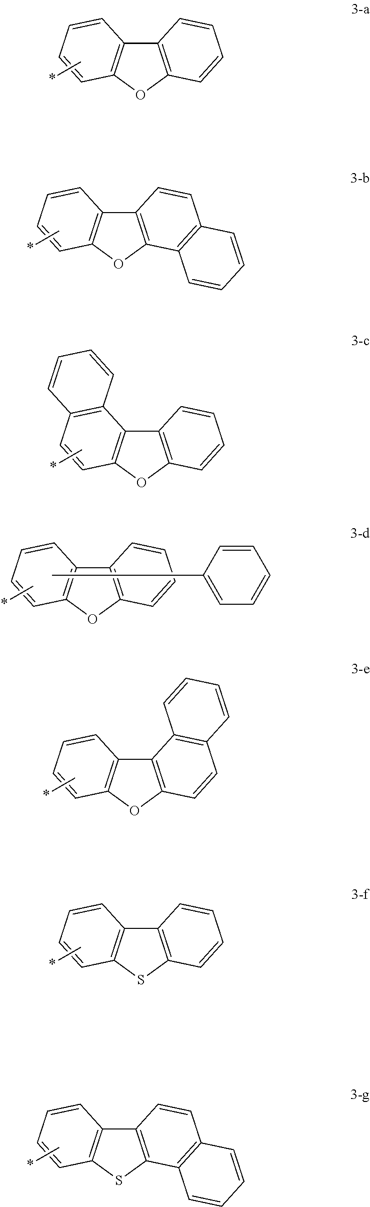

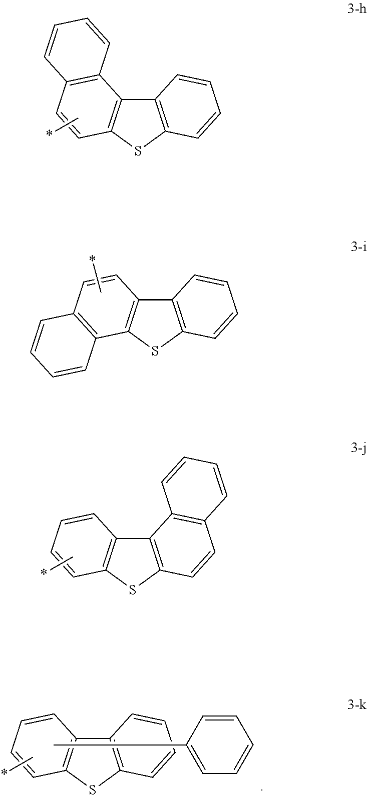

The amine compound represented by Formula 1 of an embodiment may be represented by Formula 1-1. Formula 1-1 is the embodiment in which at the amine compound of an embodiment in which Formula 3 is bonded to Ar₁ and Ar₂ in Formula 1 is specified. In Formula 1-1, the same as described in Formula 1 may be applied to R₁ to R₃, a, b, and L₁ to L₃. In Formula 1-1, the same as described in Formula 3 may be applied to X, R₉, R₁₀, h and i. In Formula 1-1 of an embodiment, a plurality of X's, R₉, R₁₀, h, and i may be the same as or different from each other.

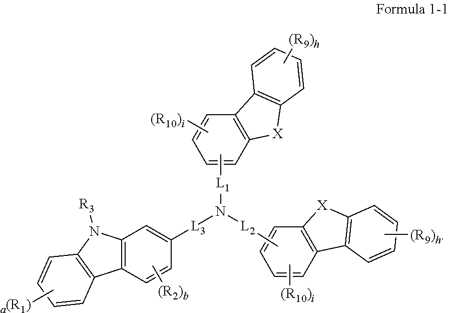

The amine compound of an embodiment may be represented by Formula 4. Formula 4 is the amine compound of an embodiment in which Formula 3 is bonded to Formula 1, and corresponds to that R₉ and R₁₀ in Formula 3 are specified. In some embodiments, Formula 4 may correspond to that R₉ and R₁₀ in Formula 1-1 are specified. In Formula 4, R₁ to R₃, a, b, and L₁ to L₃ may each independently be the same as defined in Formula 1.

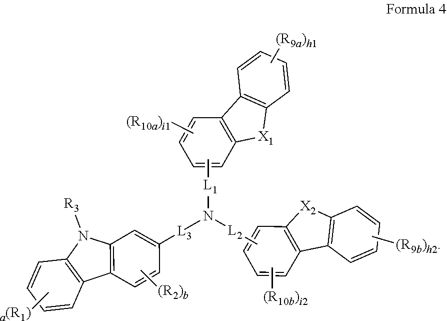

In Formula 4, X₁ and X₂ may each independently be O or S. h1 and h2 may each independently be an integer of 0 to 4, and i1 and i2 may each independently be an integer of 0 to 3. In an embodiment, the description with respect to X in Formula 3 may be applied to X₁ and X₂. In some embodiments, the description with respect to h in Formula 3 may be applied to h1 and h2, and the description with respect to i in Formula 3 may be applied to i1 and i2.

In Formula 4, R₉ₐ and R₁₀ₐ may each independently be a hydrogen atom, a deuterium atom, or a substituted or unsubstituted aryl group having 6 to 30 ring-forming carbon atoms, and/or adjacent R₉ₐs or adjacent R₁₀ₐs may be bonded to each other to form an aromatic hydrocarbon ring. In an embodiment, when adjacent R₉ₐs or adjacent R₁₀ₐs are bonded to each other to form an aromatic hydrocarbon ring, only one pair of R₉ₐs or R₁₀ₐs selected from among a plurality of R₉ₐs and R₁₀ₐs may be bonded to each other to form an aromatic hydrocarbon ring. For example, when adjacent R₉ₐs or adjacent R₁₀ₐs are bonded to each other to form an aromatic hydrocarbon ring, a pair of adjacent R₉ₐs may be bonded to each other to form an aromatic hydrocarbon ring, or a pair of adjacent R₁₀ₐs may be bonded to each other to form an aromatic hydrocarbon ring.

In Formula 4, R_{9b} and R_{10b} may each independently be a hydrogen atom, a deuterium atom, or a substituted or unsubstituted aryl group having 6 to 30 ring-forming carbon atoms, and/or adjacent R_{9b}s or adjacent R_{10b}s may be bonded to each other to form an aromatic hydrocarbon ring. In an embodiment, when adjacent R₉ₐs or adjacent R₁₀ₐs are bonded to each other to form an aromatic hydrocarbon ring, only one pair of R₉ₐs or R₁₀ₐs selected from among a plurality of R₉ₐs and R₁₀ₐs may be bonded to each other to form an aromatic hydrocarbon ring. For example, when adjacent R₉ₐs or adjacent R₁₀ₐs are bonded to each other to form an aromatic hydrocarbon ring, a pair of adjacent R₉ₐs may be bonded to each other to form an aromatic hydrocarbon ring, or a pair of adjacent R₁₀ₐs may be bonded to each other to form an aromatic hydrocarbon ring. In this embodiment, the amine compound represented by Formula 4 of an embodiment may include a naphthobenzofuran or naphthobenzothiophene moiety.

In an embodiment, Formula 4 may be represented by any one selected from among Formula 4-1 to Formula 4-5. Formula 4-1 and Formula 4-5 correspond to that R₁ and R₂ in Formula 4 are specified. For example, Formula 4-1 may correspond to the embodiment in which a and b are 0, or the embodiment in which a is 4, b is 3, and R₁ and R₂ are all hydrogen atoms. Formula 4-2 to Formula 4-4 correspond to the embodiment in which two adjacent R₁s are bonded to each other to form an aromatic hydrocarbon ring, and Formula 4-5 corresponds to the embodiment in which adjacent R₁ and R₂ are bonded to each other to form an aromatic hydrocarbon ring. In Formula 4-1 to Formula 4-5, the same as described in Formula 1 may be applied to R₃, and L₁ to L₃. In Formula 4-1 to Formula 4-5, the same as described in Formula 4 may be applied to X₁, X₂, R₉ₐ, R_{9b}, R₁₀ₐ, R_{10b}, h1, h2, i1, and i2.

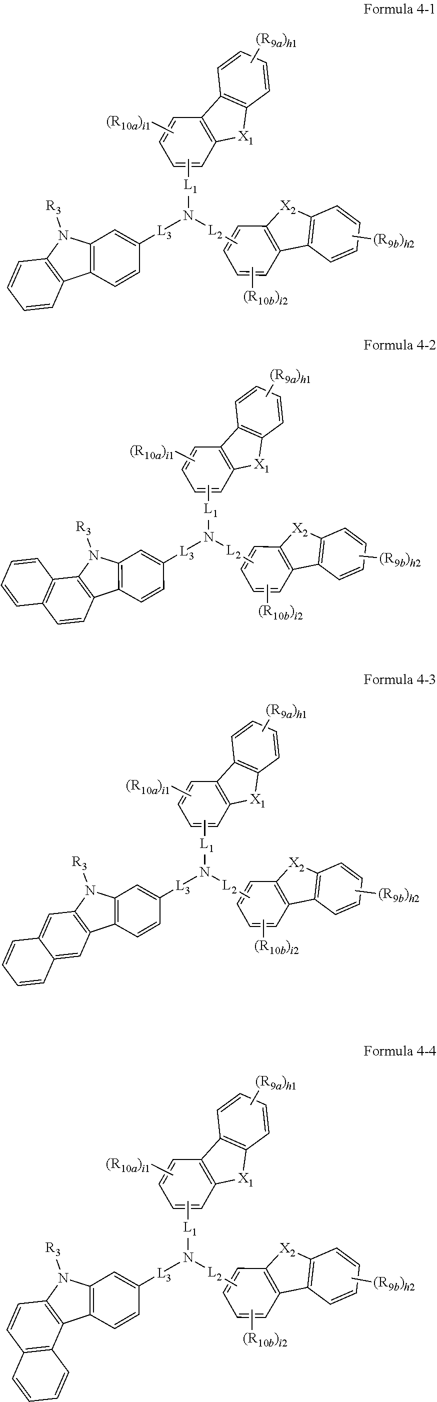

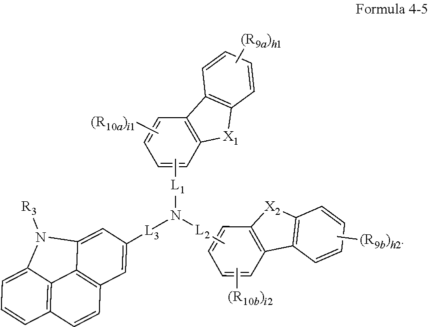

The amine compound represented by Formula 1 of an embodiment may be one selected from among the compounds of Compound Group 1. The hole transport region HTR of the light emitting element ED of an embodiment may include at least one selected from among the amine compounds disclosed in Compound Group 1. D in Compound Group 1 is a deuterium atom.

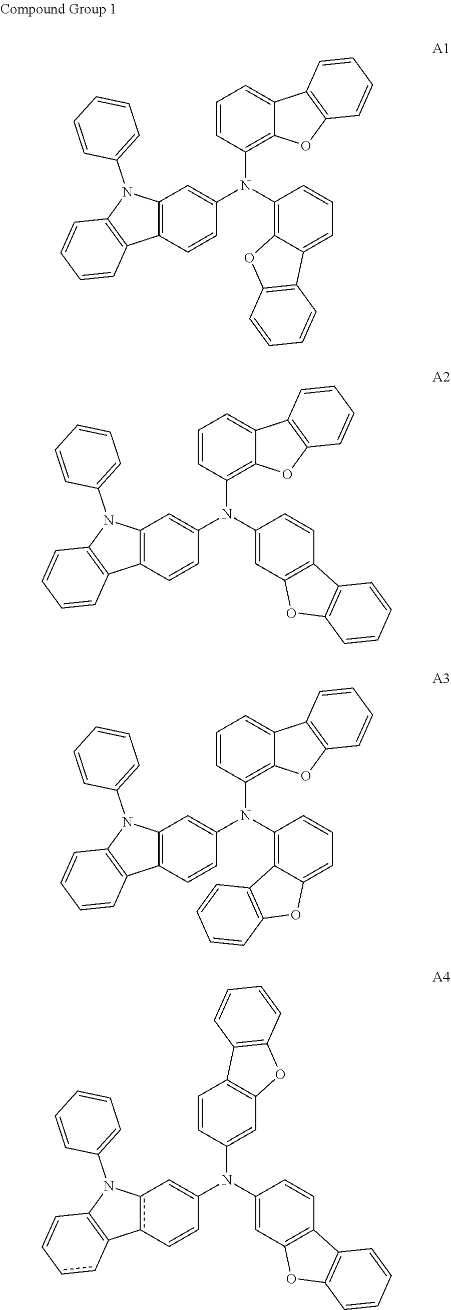

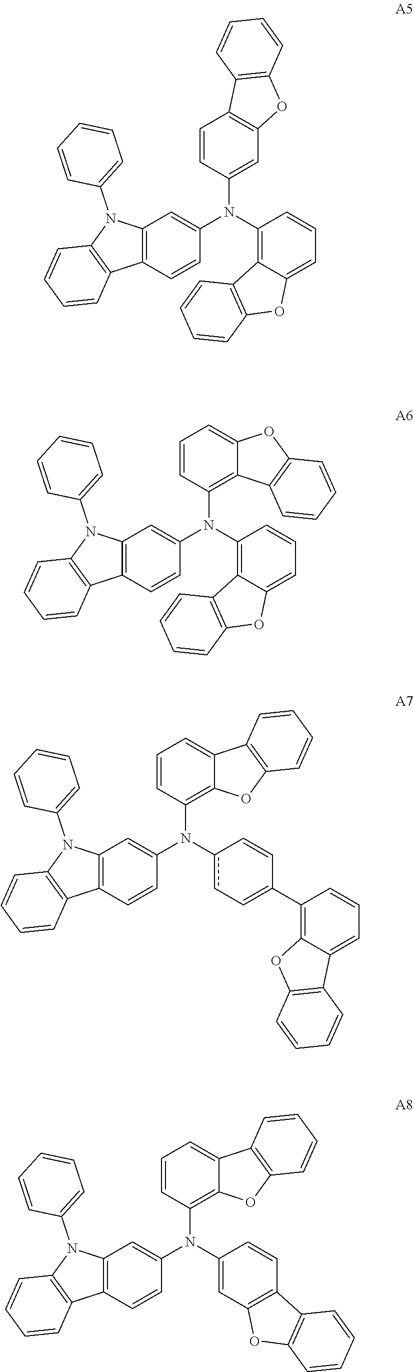

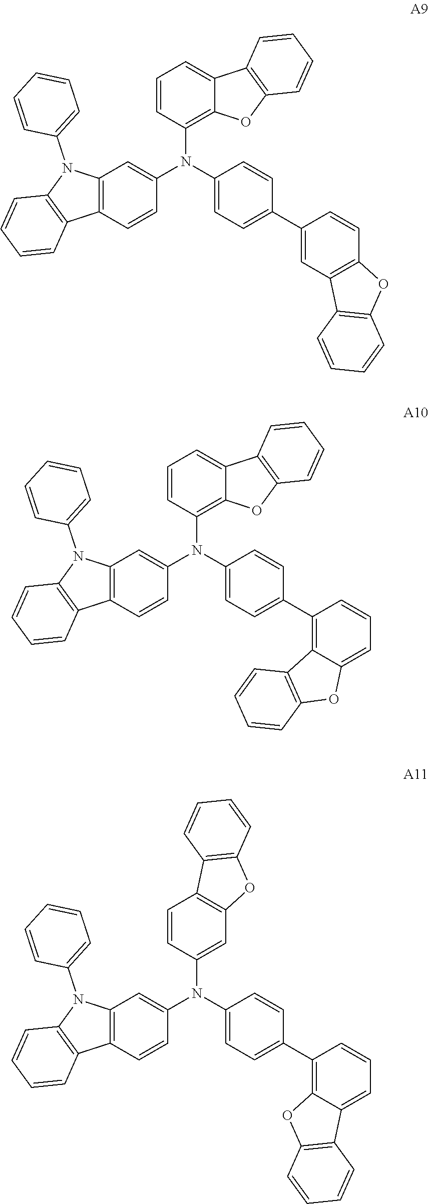 [SMILES: c1ccc(-n2c3ccccc3c3ccc(N(c4ccc(-c5ccc6oc7ccccc7c6c5)cc4)c4cccc5c4oc4ccccc45)cc32)cc1 ; c1ccc(-n2c3ccccc3c3ccc(N(c4ccc(-c5cccc6oc7ccccc7c56)cc4)c4cccc5c4oc4ccccc45)cc32)cc1 ; c1ccc(-n2c3ccccc3c3ccc(N(c4ccc(-c5cccc6c5oc5ccccc56)cc4)c4ccc5c(c4)oc4ccccc45)cc32)cc1]

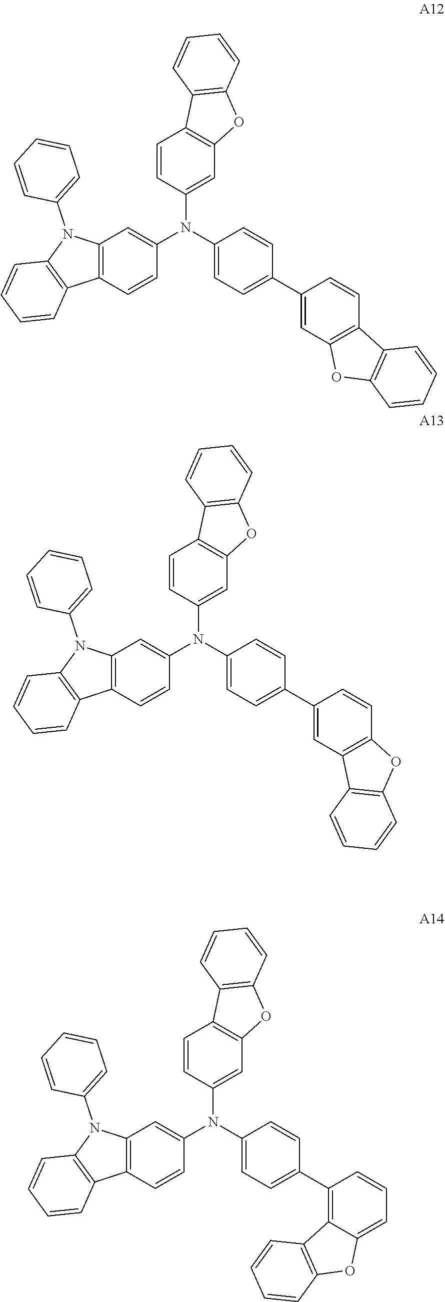 [SMILES: c1ccc(-n2c3ccccc3c3ccc(N(c4ccc(-c5ccc6c(c5)oc5ccccc56)cc4)c4ccc5c(c4)oc4ccccc45)cc32)cc1 ; c1ccc(-n2c3ccccc3c3ccc(N(c4ccc(-c5ccc6oc7ccccc7c6c5)cc4)c4ccc5c(c4)oc4ccccc45)cc32)cc1 ; c1ccc(-n2c3ccccc3c3ccc(N(c4ccc(-c5cccc6oc7ccccc7c56)cc4)c4ccc5c(c4)oc4ccccc45)cc32)cc1]

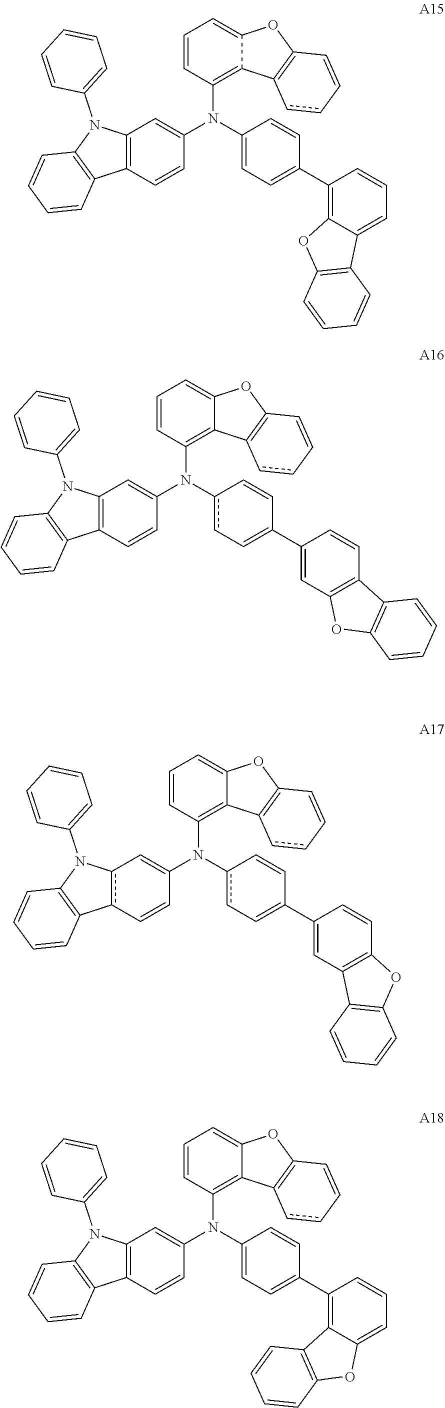

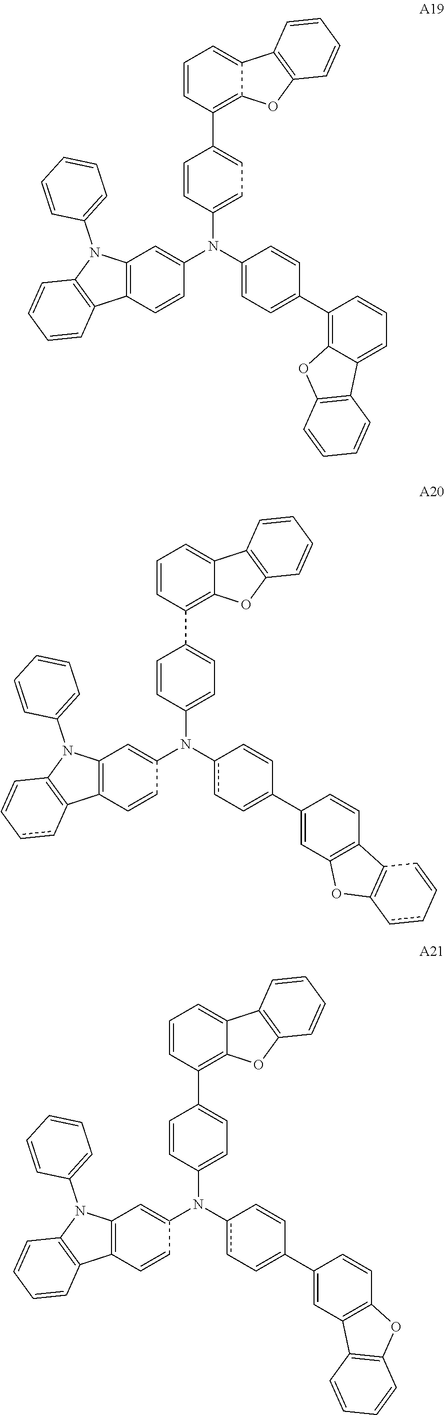

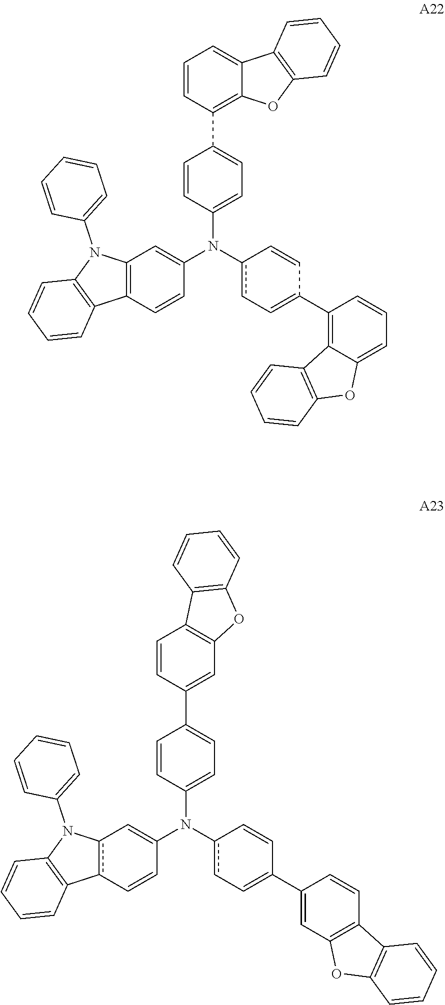

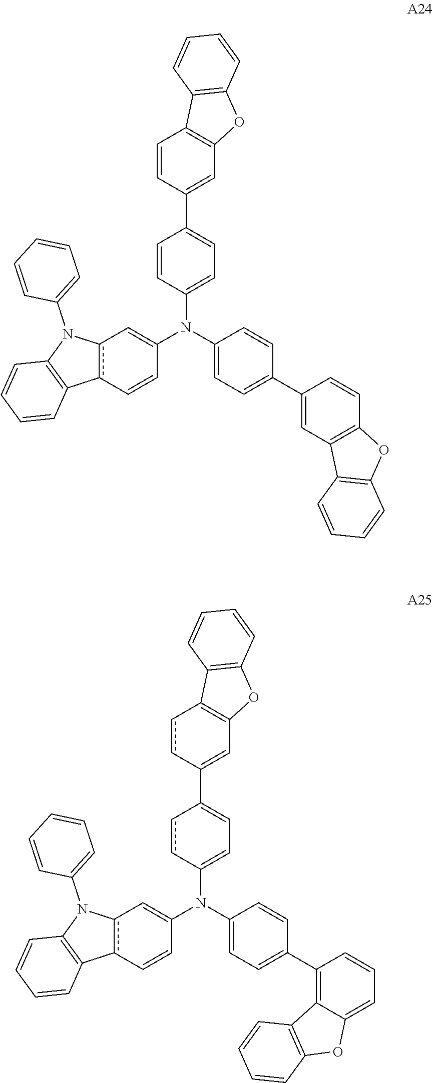

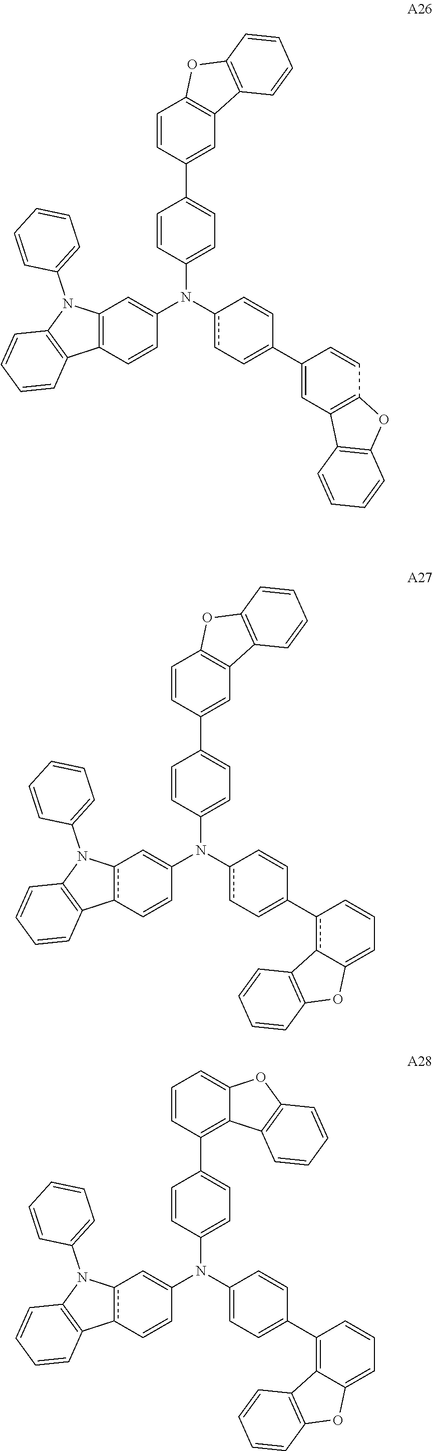

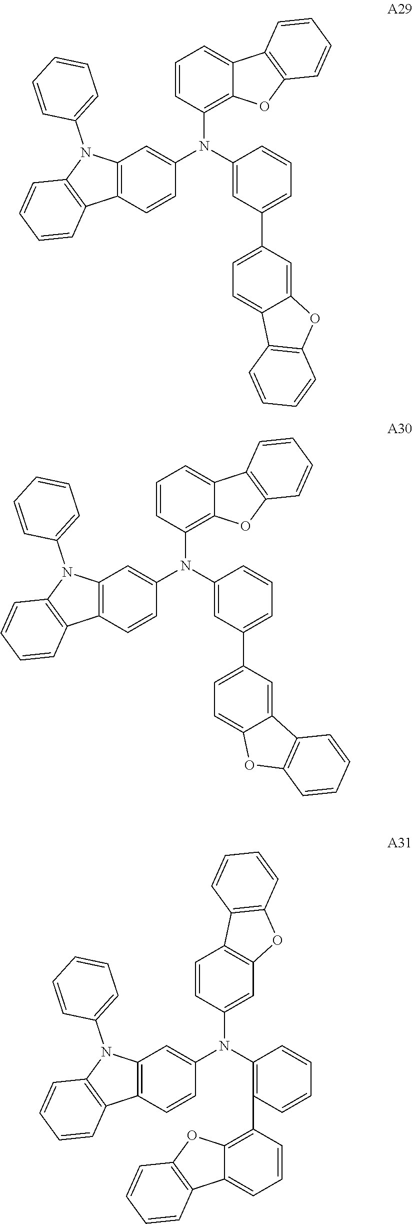

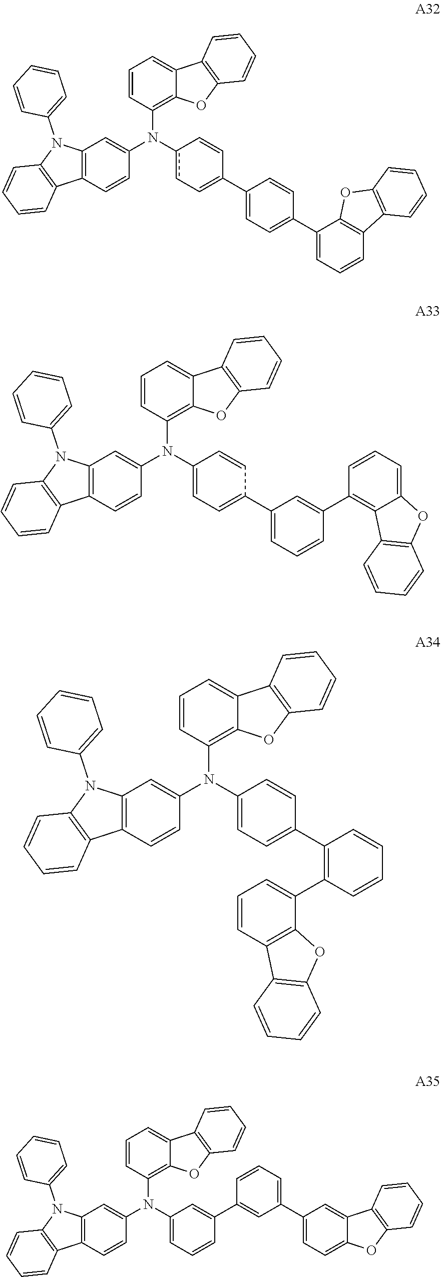

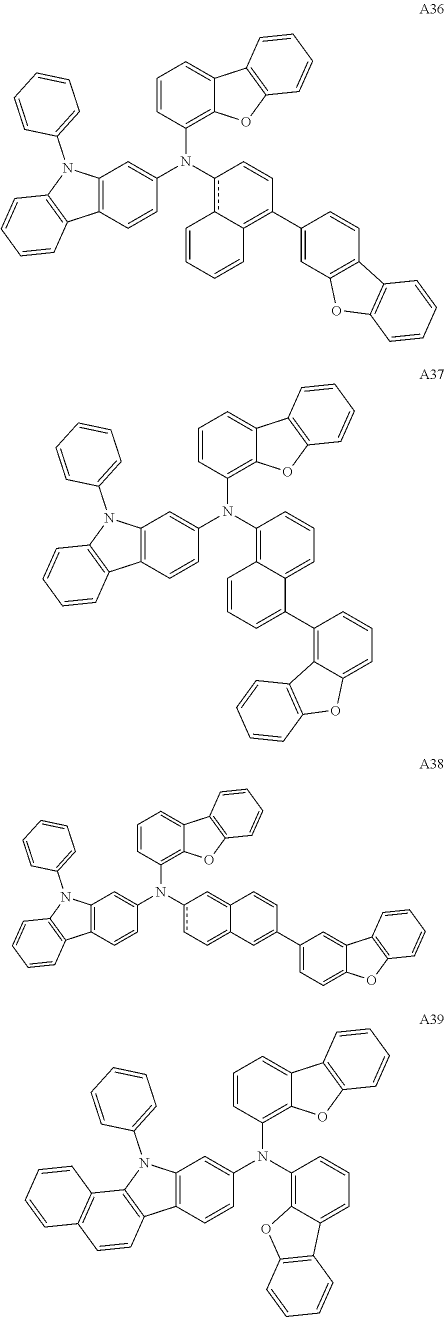

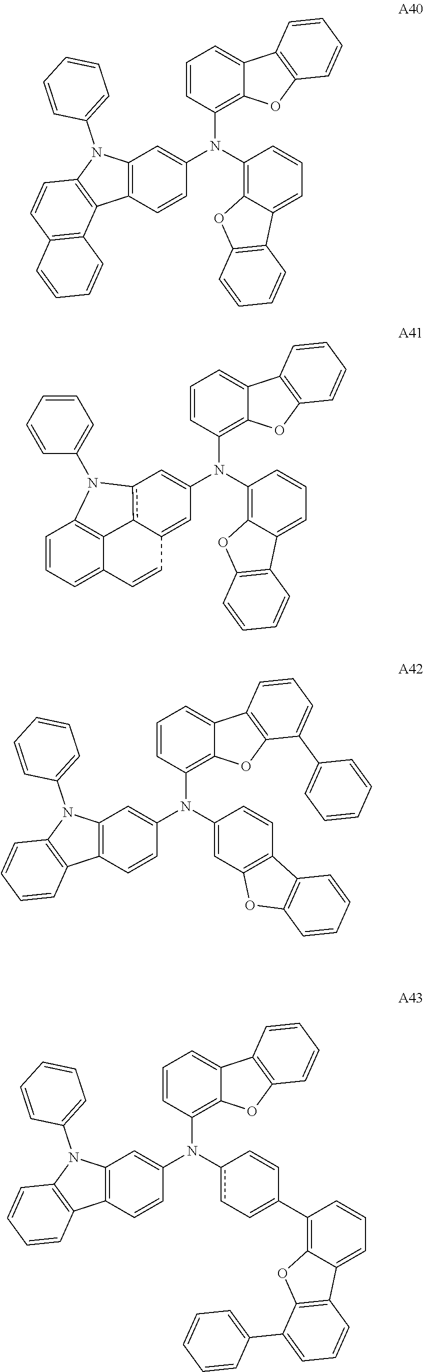

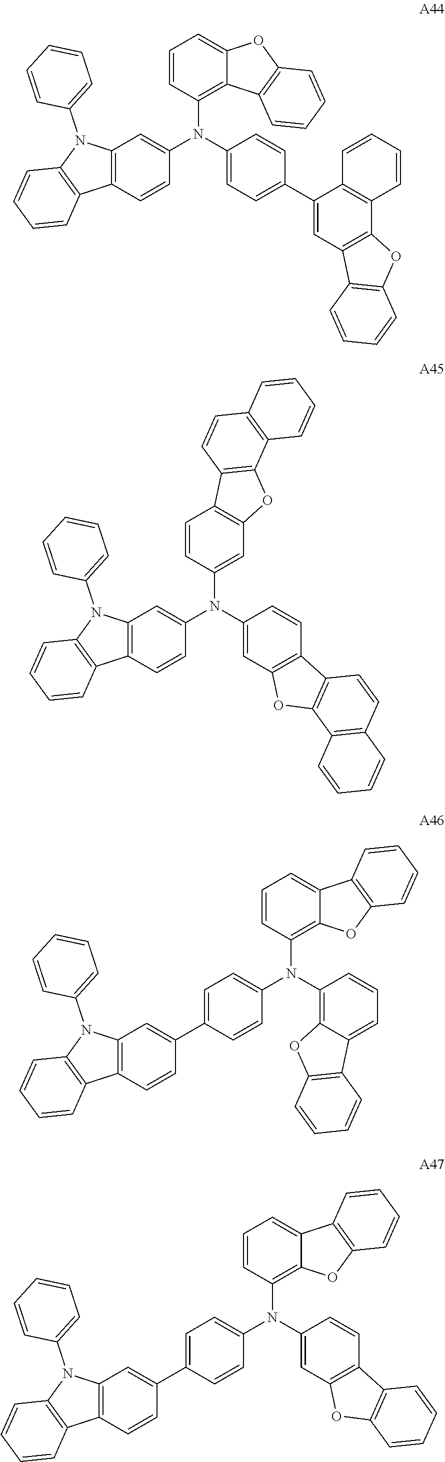 [SMILES: c1ccc(-n2c3ccccc3c3ccc(N(c4ccc(-c5cc6c7ccccc7oc6c6ccccc56)cc4)c4cccc5oc6ccccc6c45)cc32)cc1 ; c1ccc(-n2c3ccccc3c3ccc(N(c4ccc5c(c4)oc4c6ccccc6ccc54)c4ccc5c(c4)oc4c6ccccc6ccc54)cc32)cc1 ; c1ccc(-n2c3ccccc3c3ccc(-c4ccc(N(c5cccc6c5oc5ccccc56)c5cccc6c5oc5ccccc56)cc4)cc32)cc1 ; c1ccc(-n2c3ccccc3c3ccc(-c4ccc(N(c5ccc6c(c5)oc5ccccc56)c5cccc6c5oc5ccccc56)cc4)cc32)cc1]

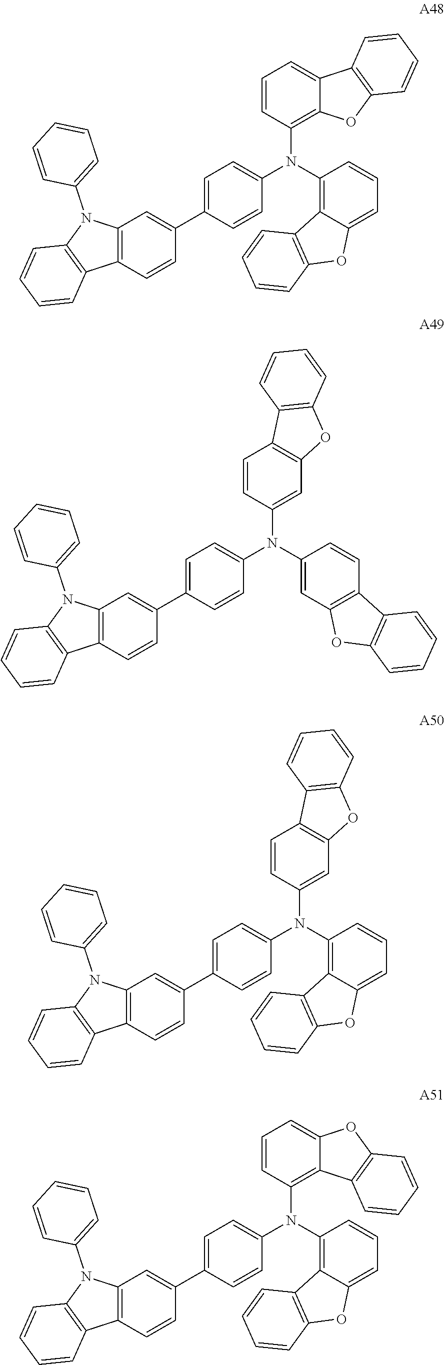 [SMILES: c1ccc(-n2c3ccccc3c3ccc(-c4ccc(N(c5cccc6c5oc5ccccc56)c5cccc6oc7ccccc7c56)cc4)cc32)cc1 ; c1ccc(-n2c3ccccc3c3ccc(-c4ccc(N(c5ccc6c(c5)oc5ccccc56)c5ccc6c(c5)oc5ccccc56)cc4)cc32)cc1 ; c1ccc(-n2c3ccccc3c3ccc(-c4ccc(N(c5ccc6c(c5)oc5ccccc56)c5cccc6oc7ccccc7c56)cc4)cc32)cc1 ; c1ccc(-n2c3ccccc3c3ccc(-c4ccc(N(c5cccc6oc7ccccc7c56)c5cccc6oc7ccccc7c56)cc4)cc32)cc1]

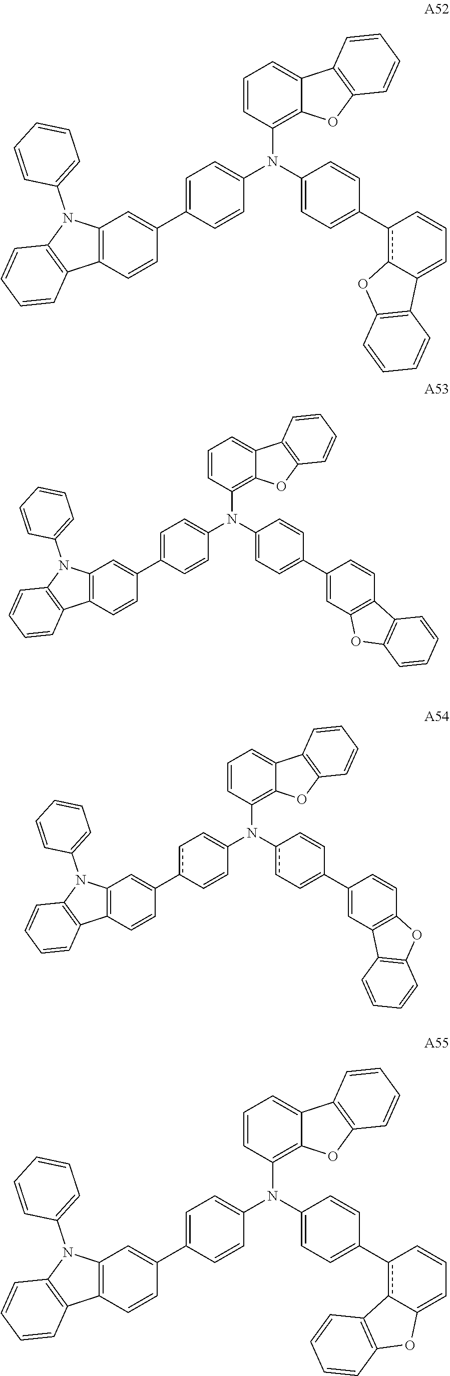

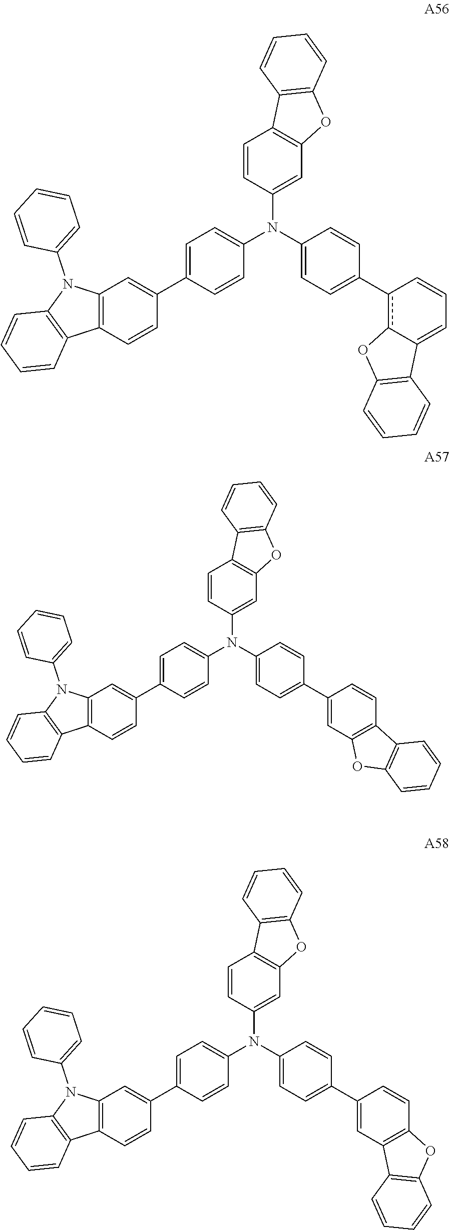

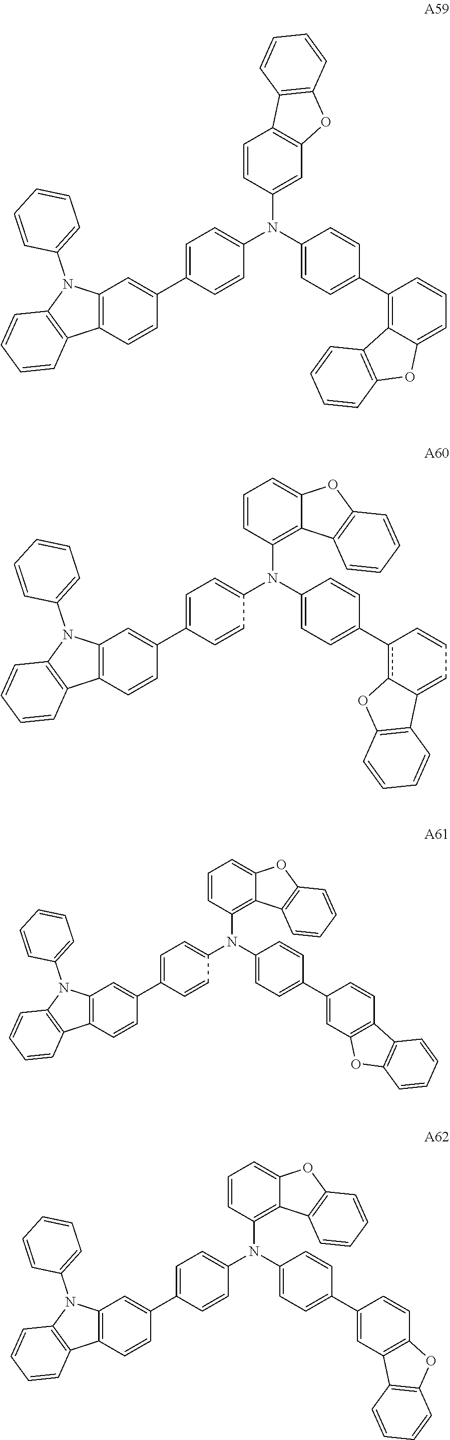

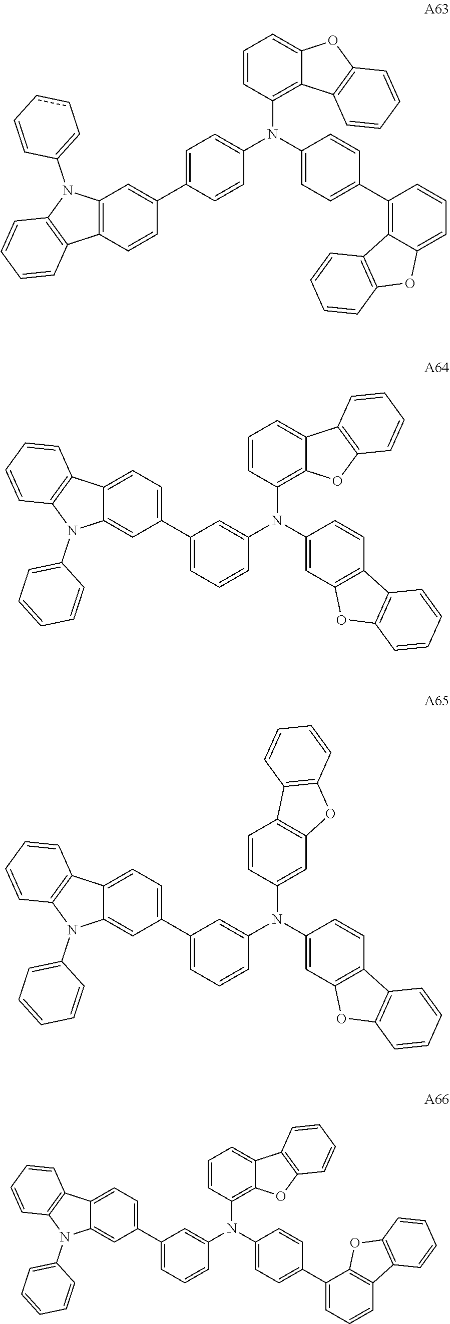

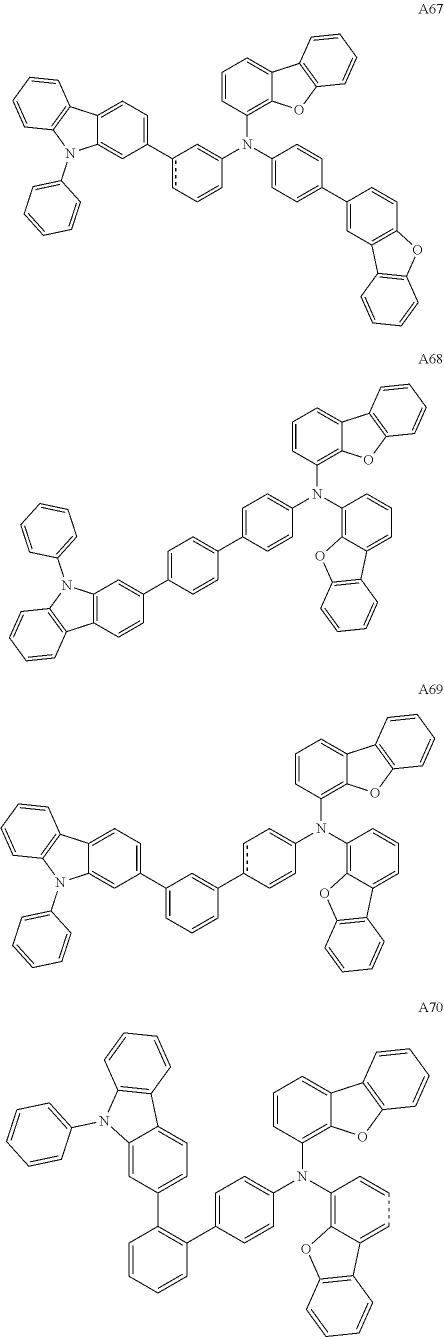

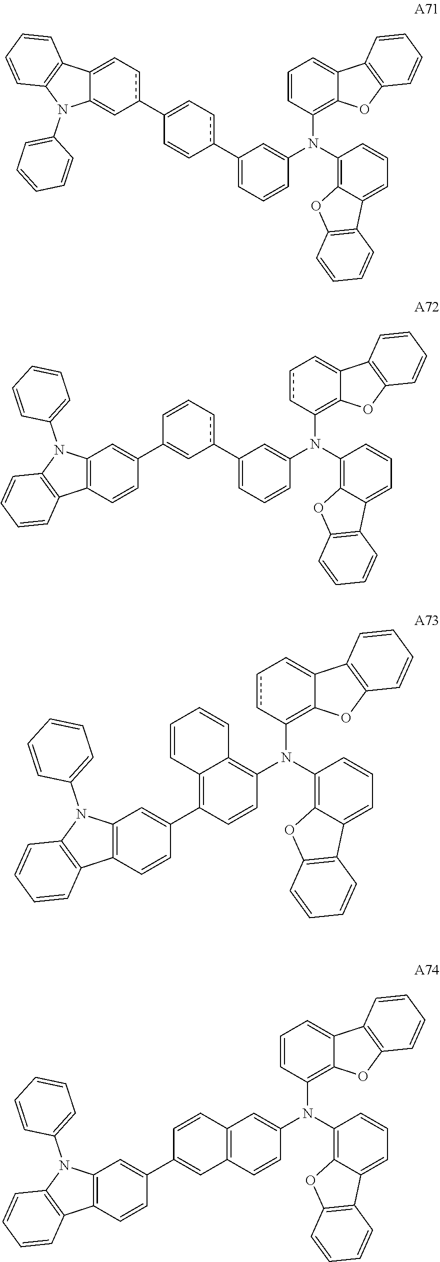

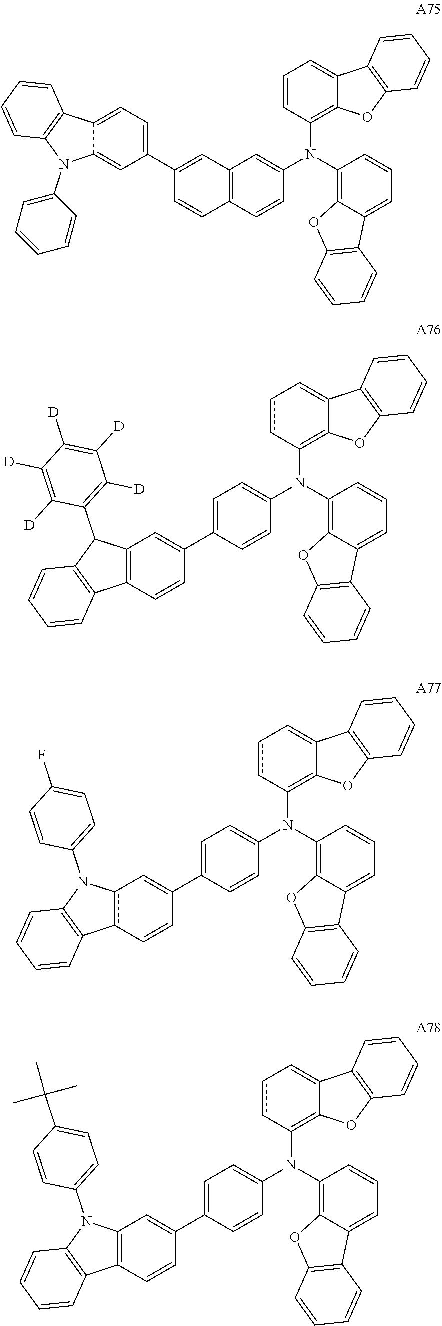

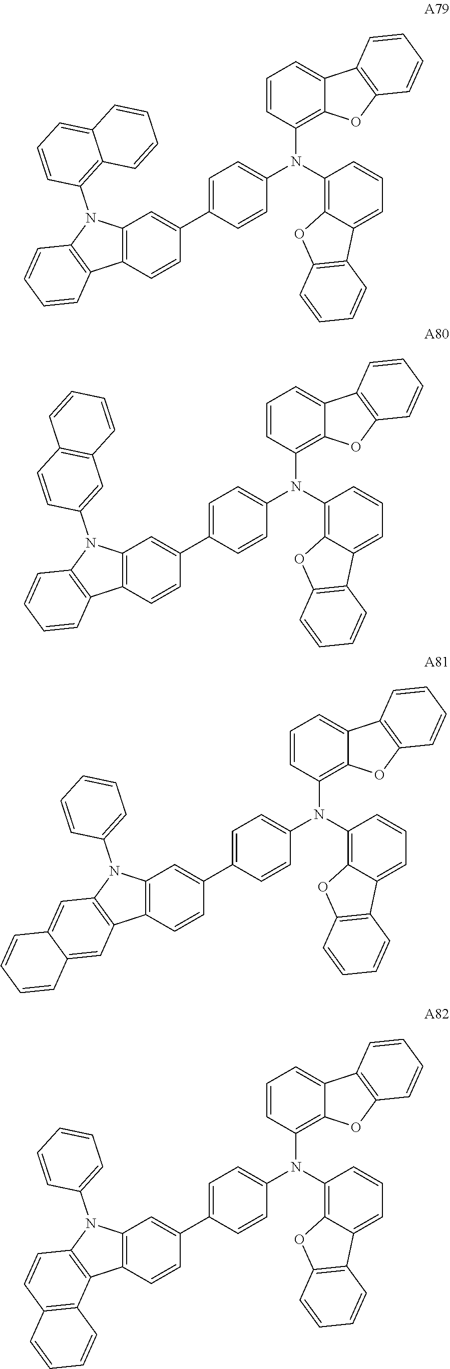 [SMILES: c1ccc2c(-n3c4ccccc4c4ccc(-c5ccc(N(c6cccc7c6oc6ccccc67)c6cccc7c6oc6ccccc67)cc5)cc43)cccc2c1 ; c1ccc2cc(-n3c4ccccc4c4ccc(-c5ccc(N(c6cccc7c6oc6ccccc67)c6cccc7c6oc6ccccc67)cc5)cc43)ccc2c1 ; c1ccc(-n2c3cc(-c4ccc(N(c5cccc6c5oc5ccccc56)c5cccc6c5oc5ccccc56)cc4)ccc3c3cc4ccccc4cc32)cc1 ; c1ccc(-n2c3cc(-c4ccc(N(c5cccc6c5oc5ccccc56)c5cccc6c5oc5ccccc56)cc4)ccc3c3c4ccccc4ccc32)cc1]

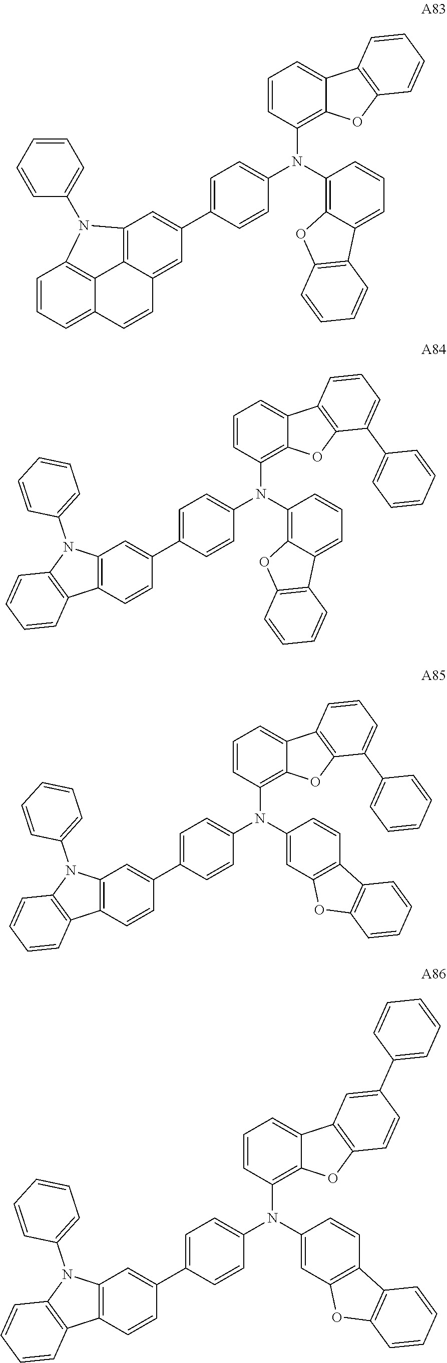 [SMILES: c1ccc(-n2c3cccc4ccc5cc(-c6ccc(N(c7cccc8c7oc7ccccc78)c7cccc8c7oc7ccccc78)cc6)cc2c5c43)cc1 ; c1ccc(-c2cccc3c2oc2c(N(c4ccc(-c5ccc6c7ccccc7n(-c7ccccc7)c6c5)cc4)c4cccc5c4oc4ccccc45)cccc23)cc1 ; c1ccc(-c2cccc3c2oc2c(N(c4ccc(-c5ccc6c7ccccc7n(-c7ccccc7)c6c5)cc4)c4ccc5c(c4)oc4ccccc45)cccc23)cc1 ; c1ccc(-c2ccc3oc4c(N(c5ccc(-c6ccc7c8ccccc8n(-c8ccccc8)c7c6)cc5)c5ccc6c(c5)oc5ccccc56)cccc4c3c2)cc1]

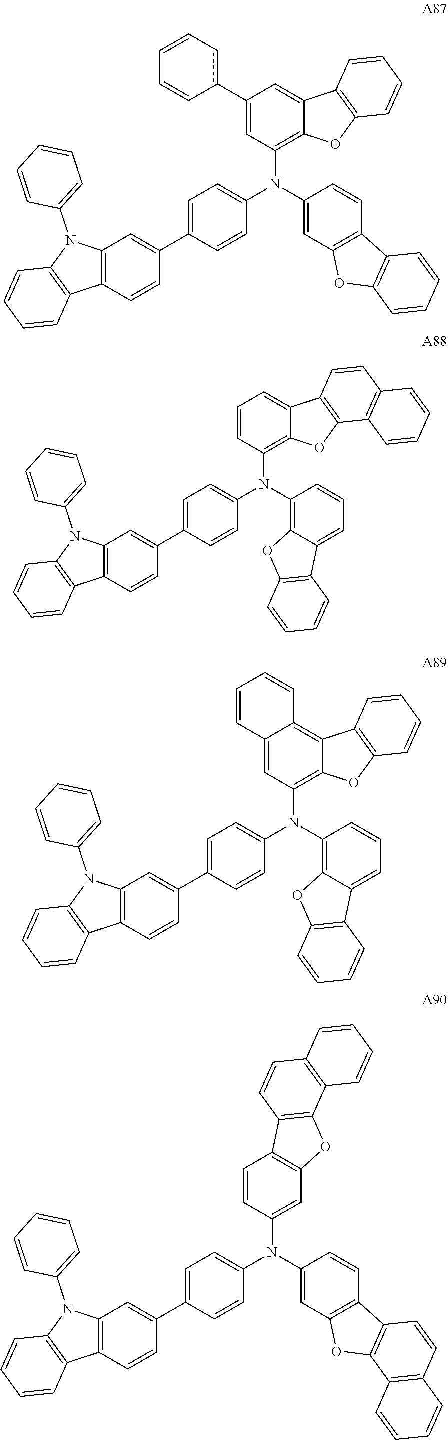

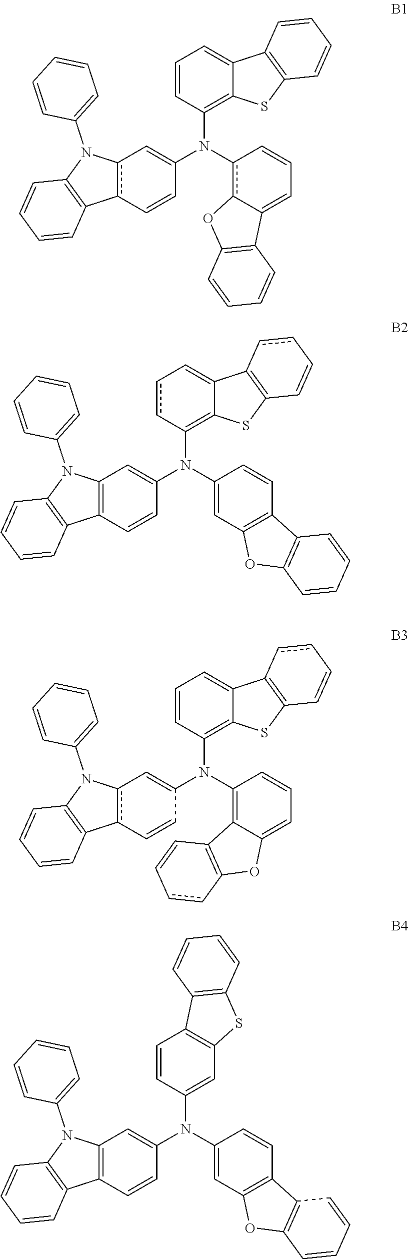

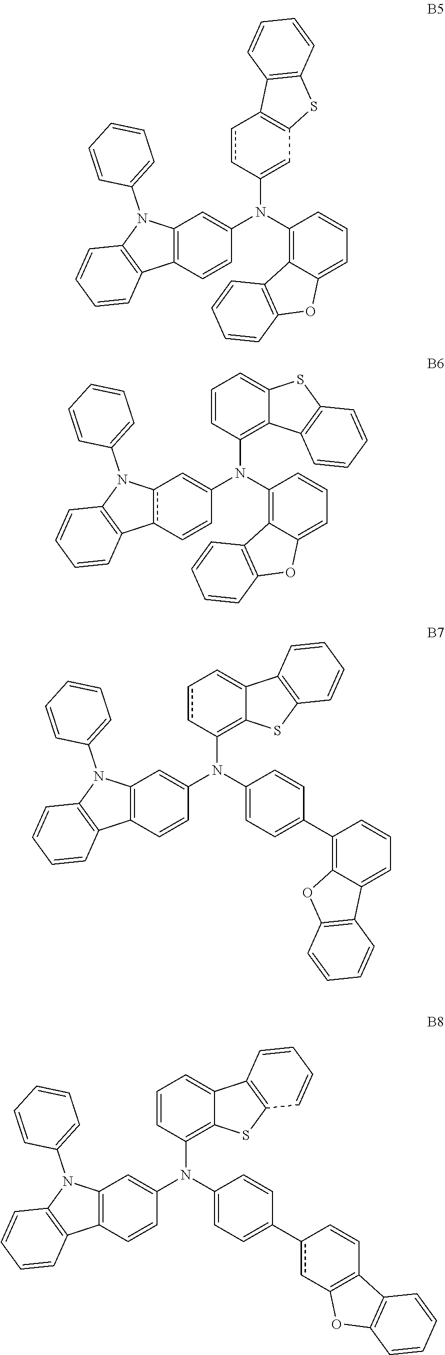

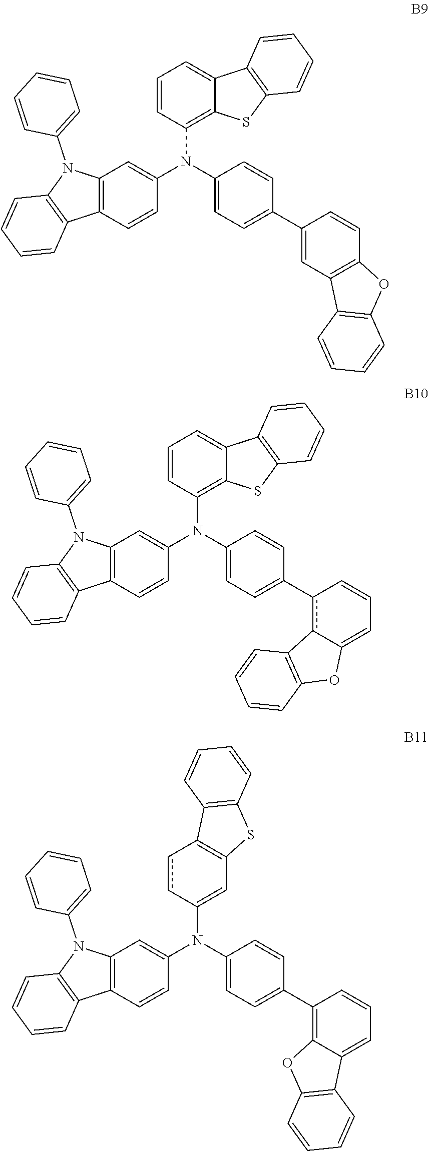

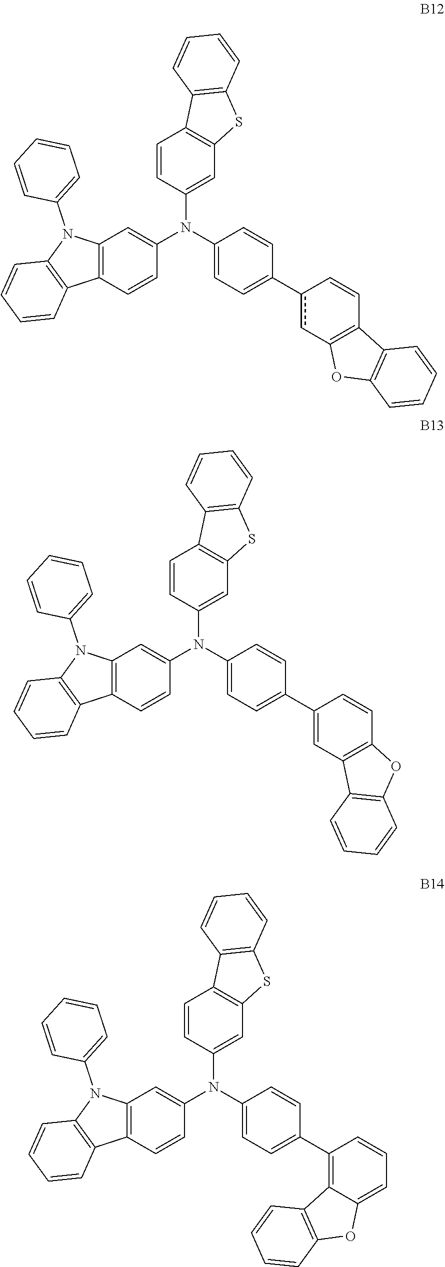

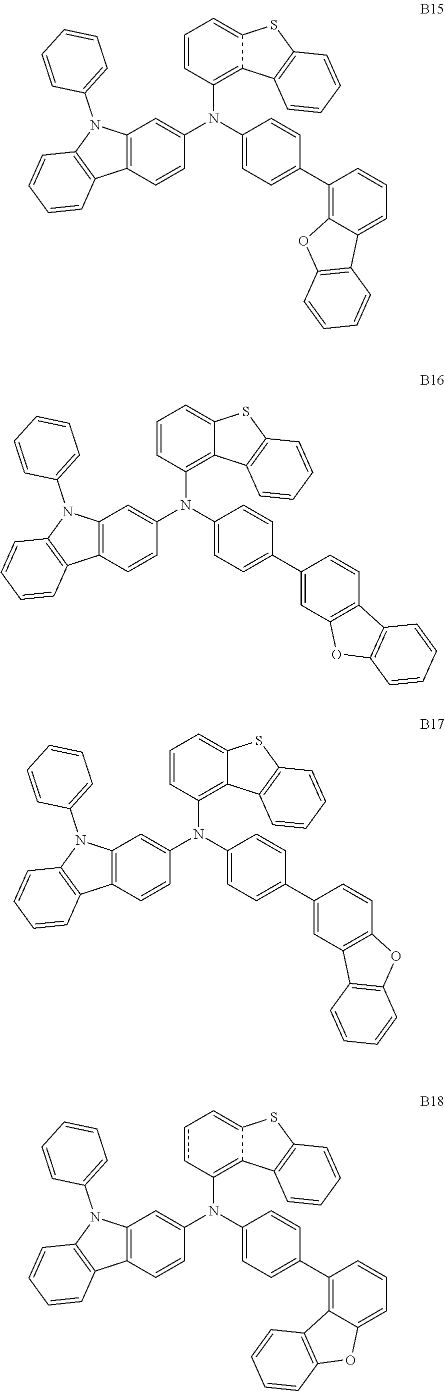

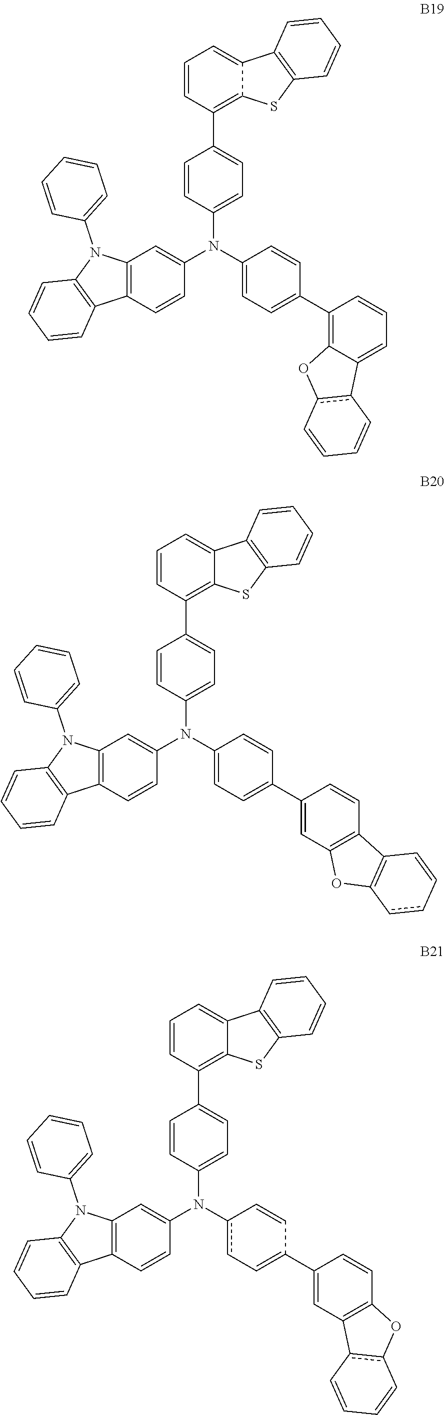

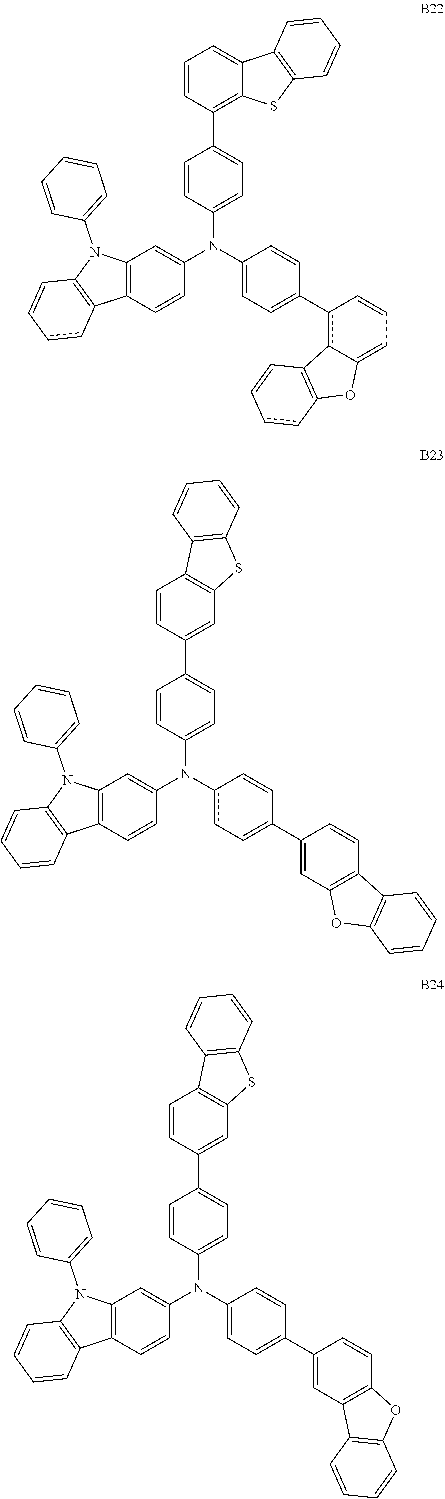

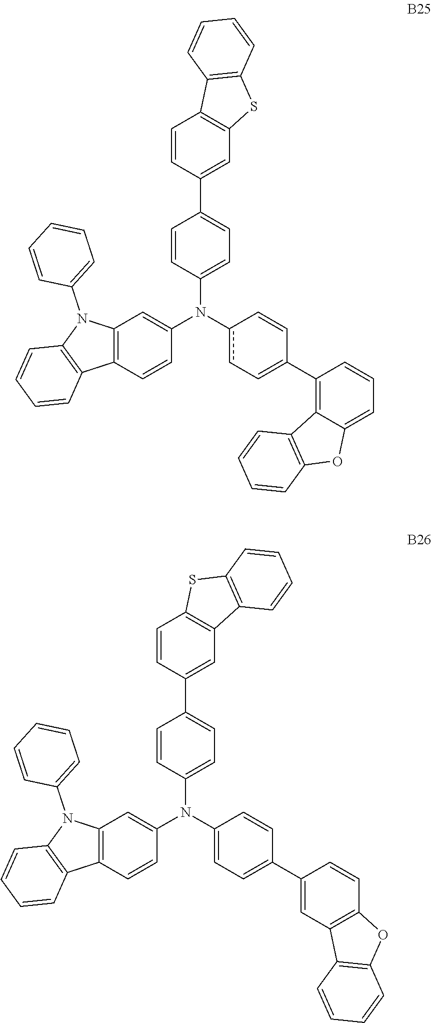

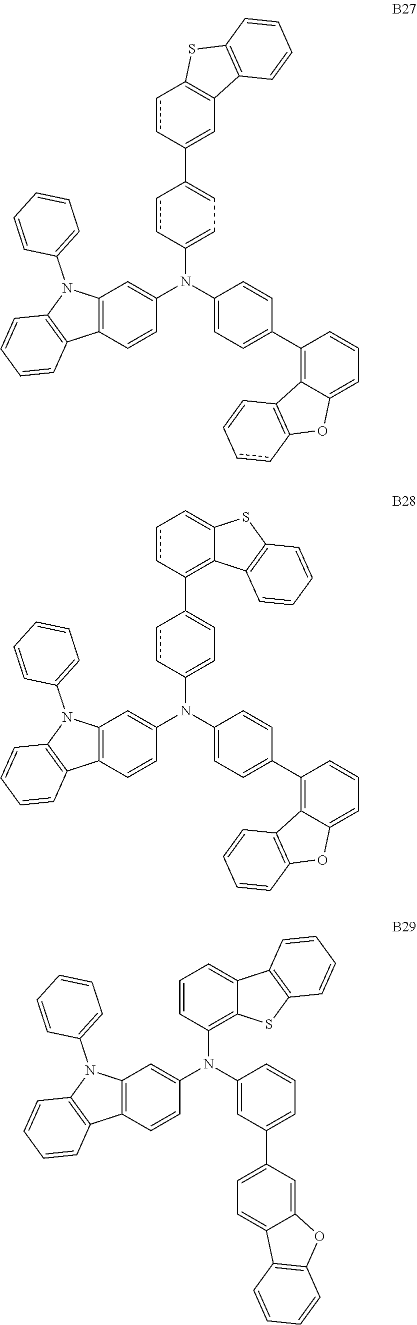

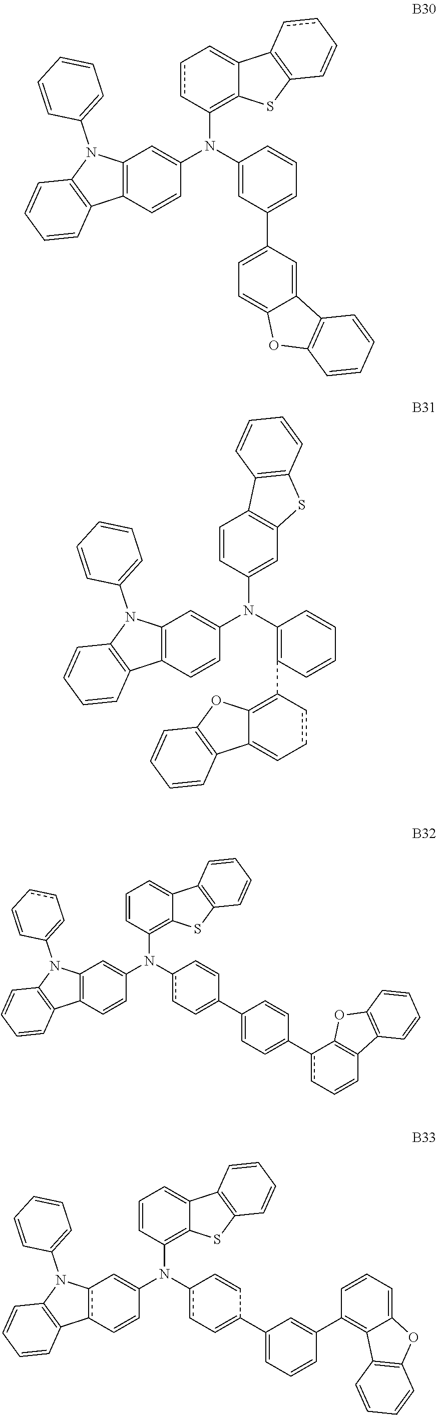

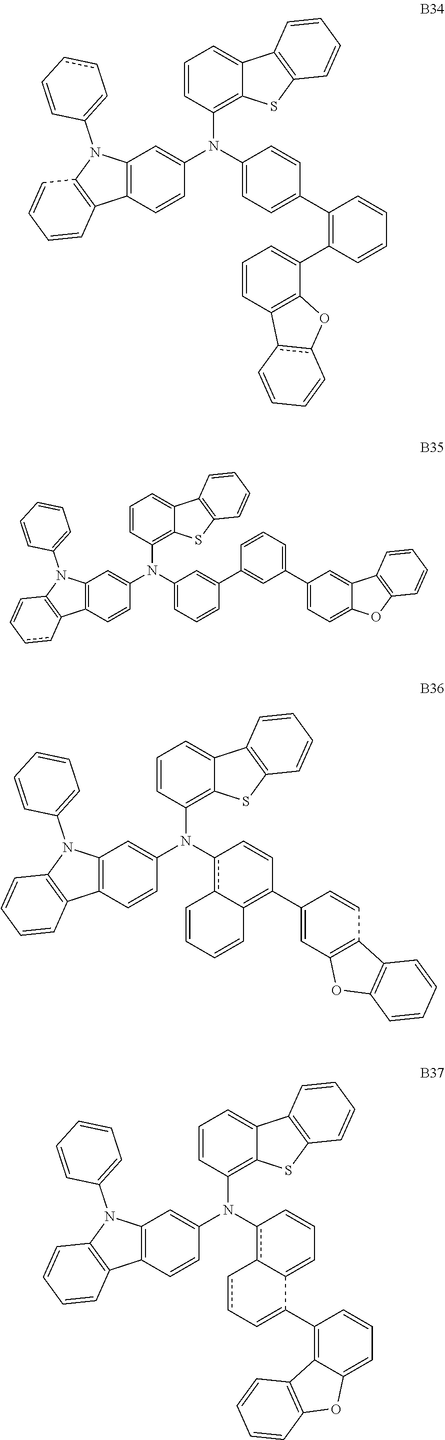

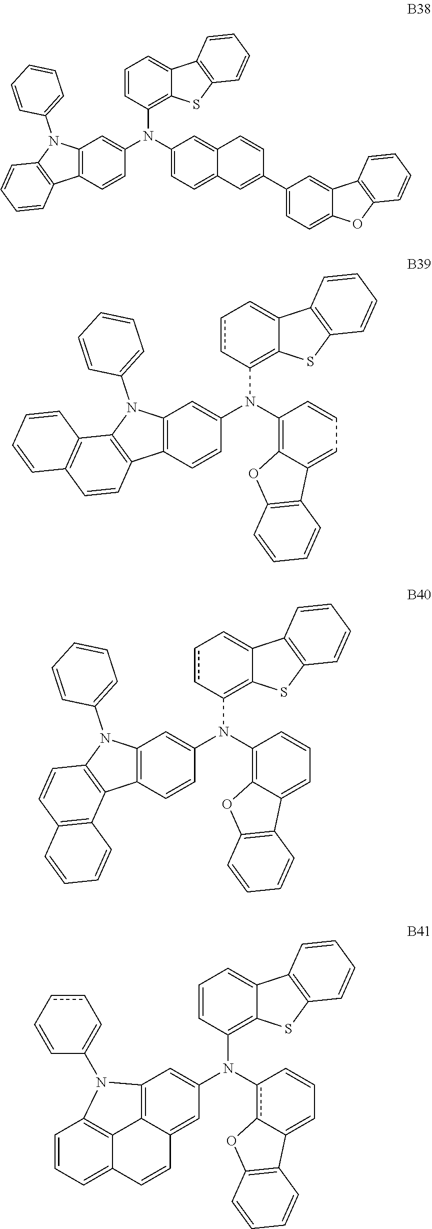

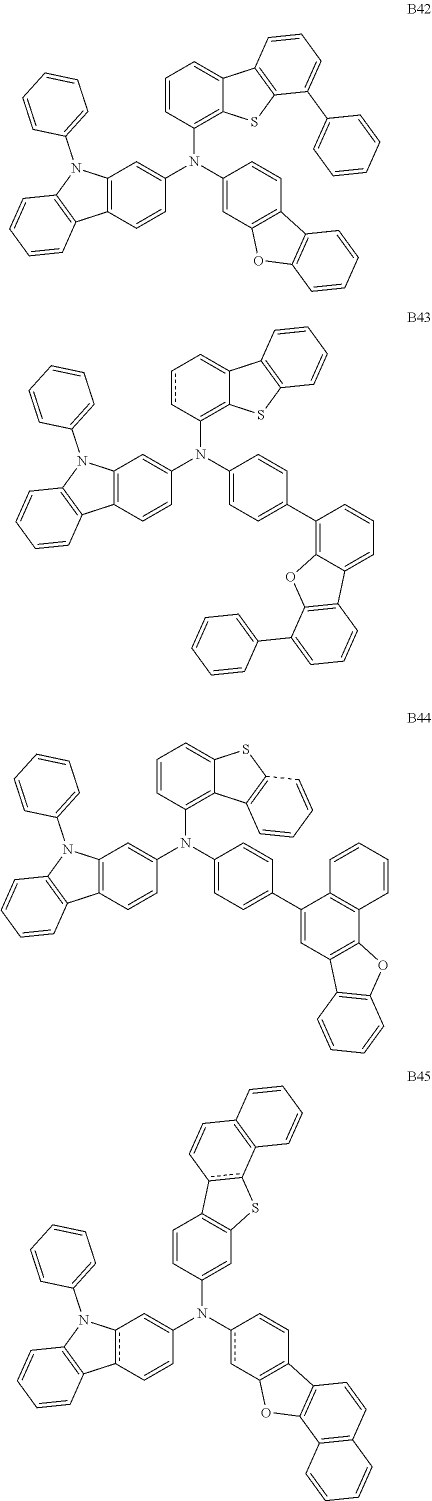

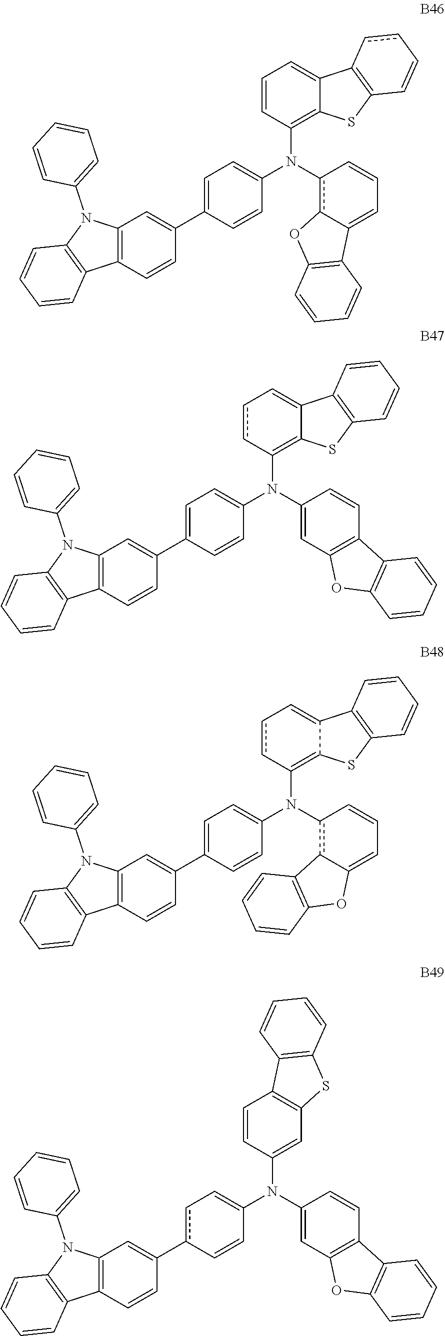

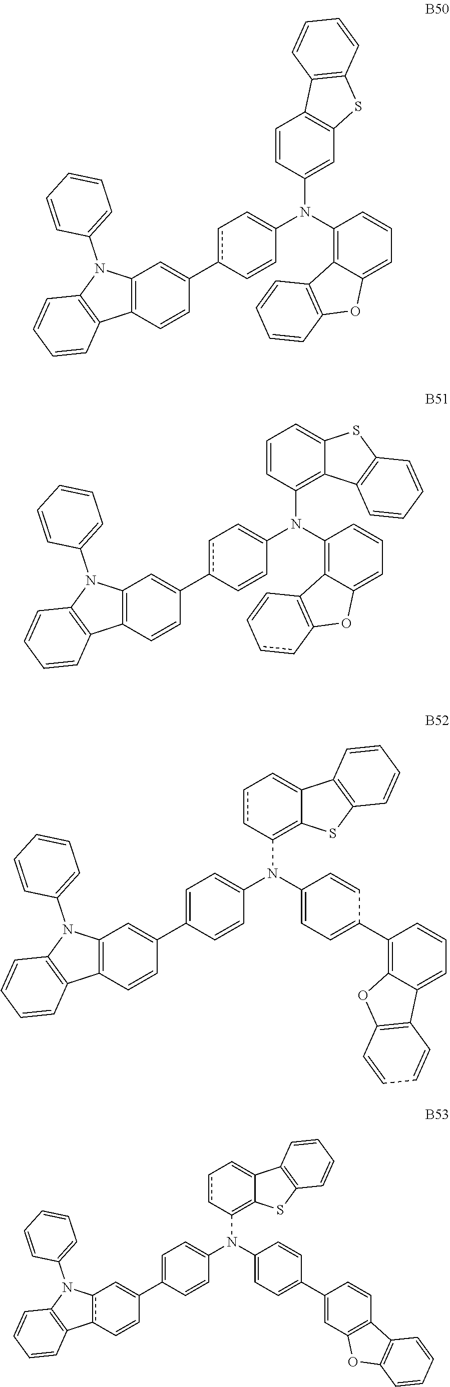

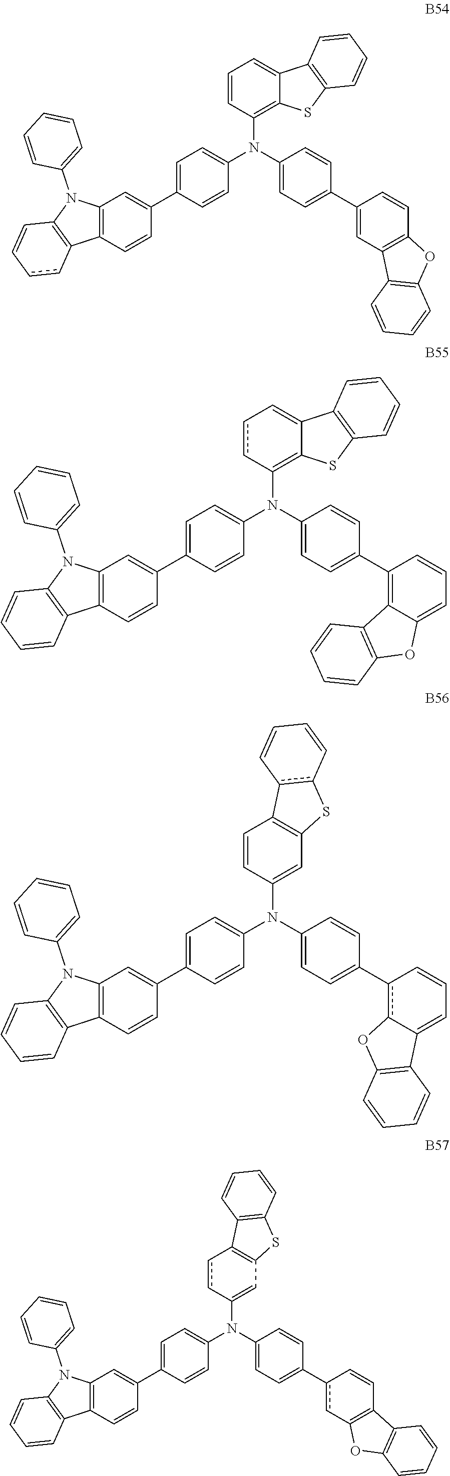

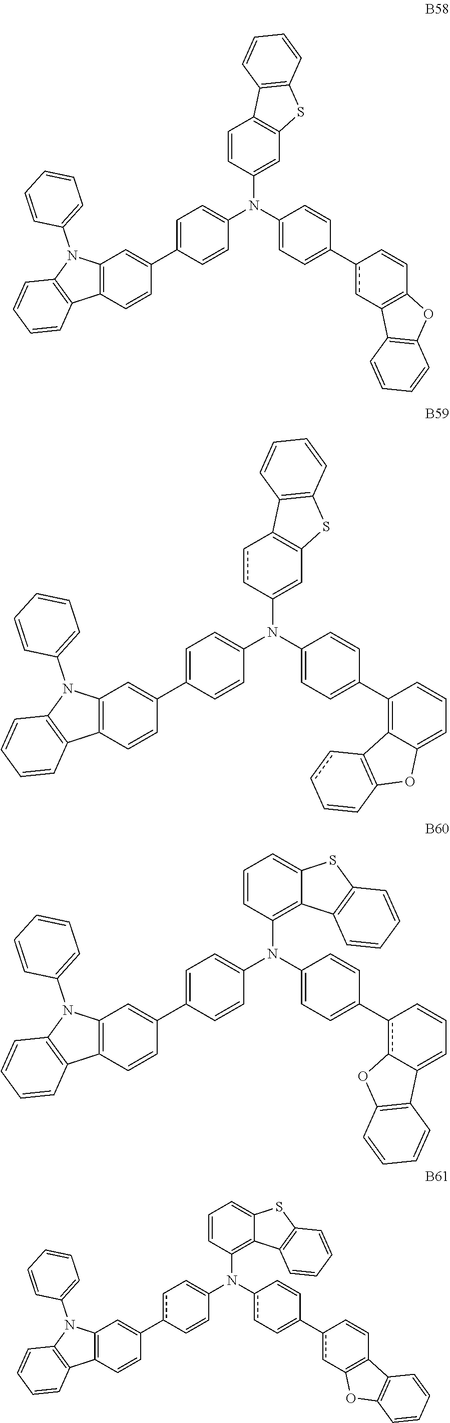

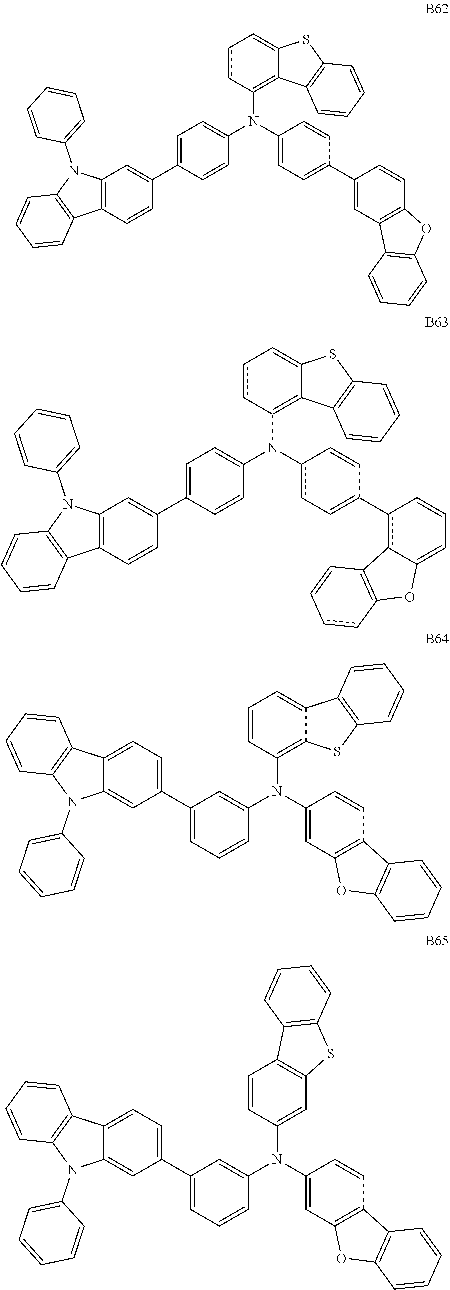

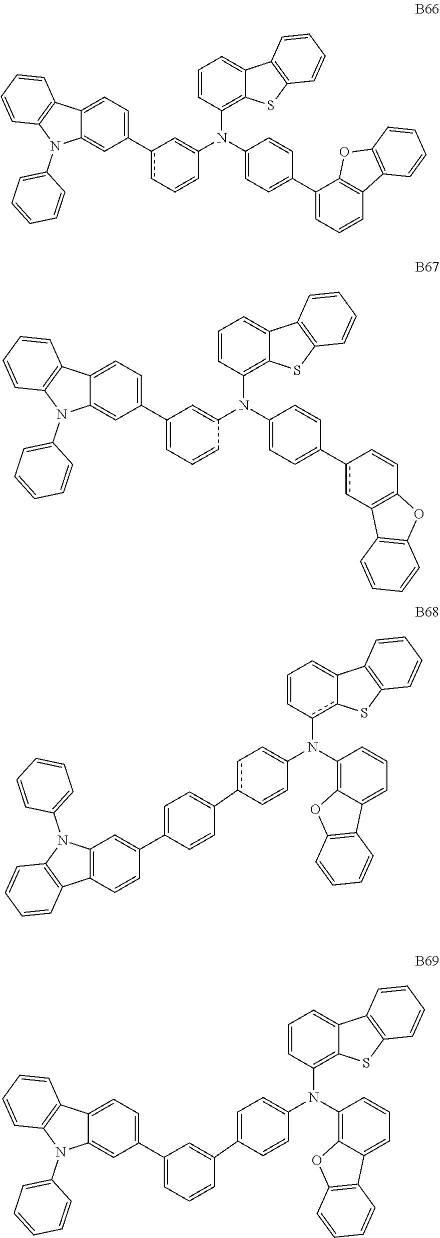

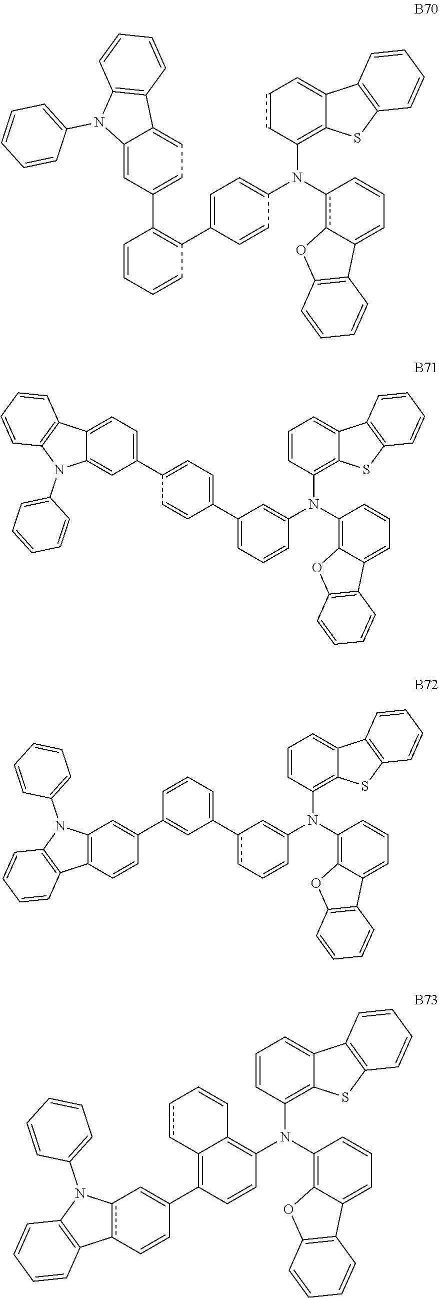

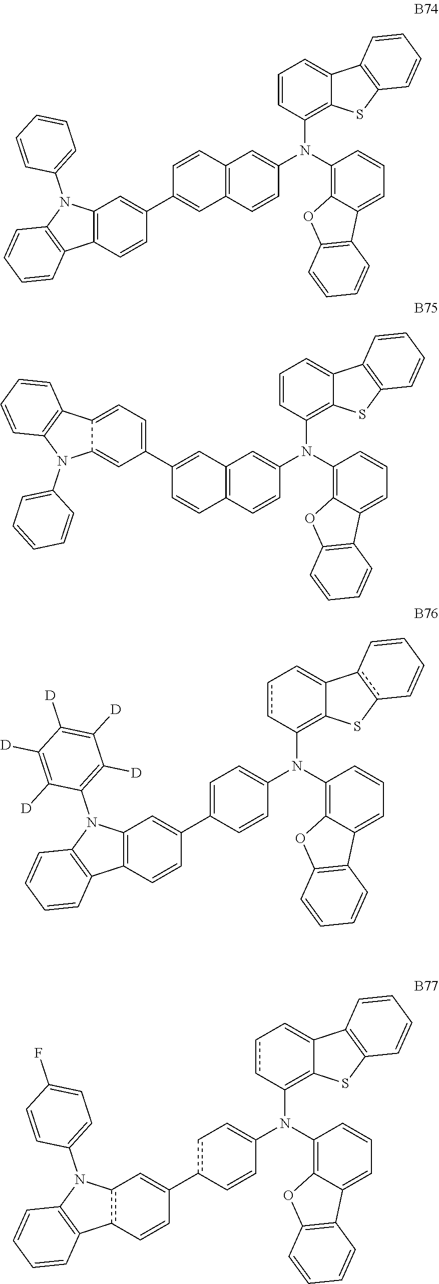

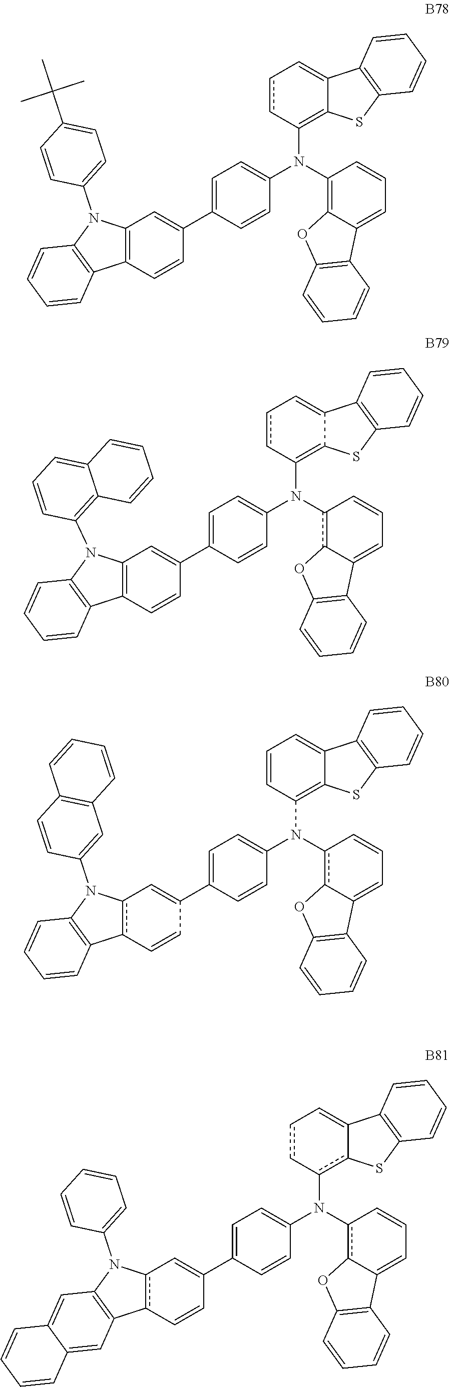

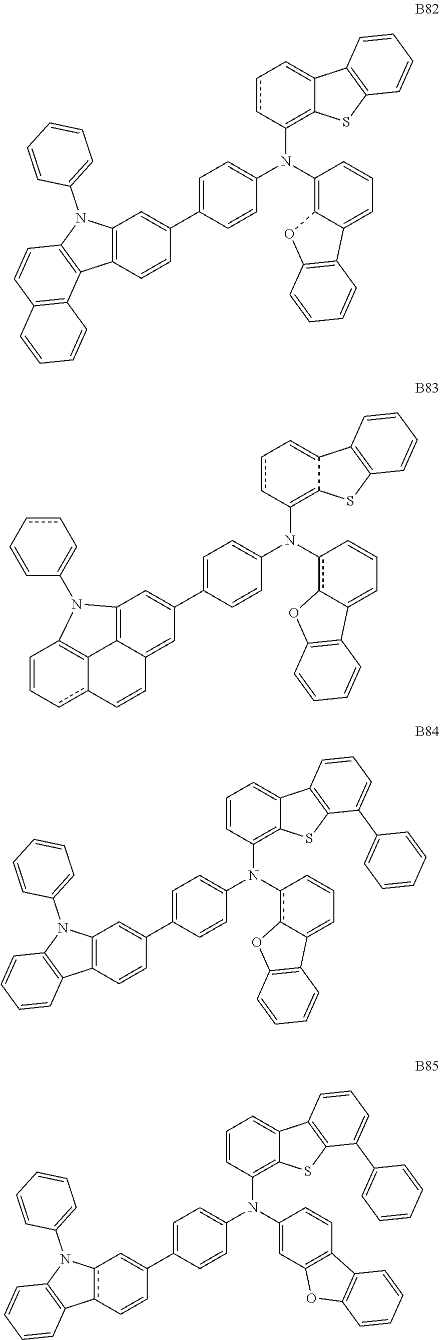

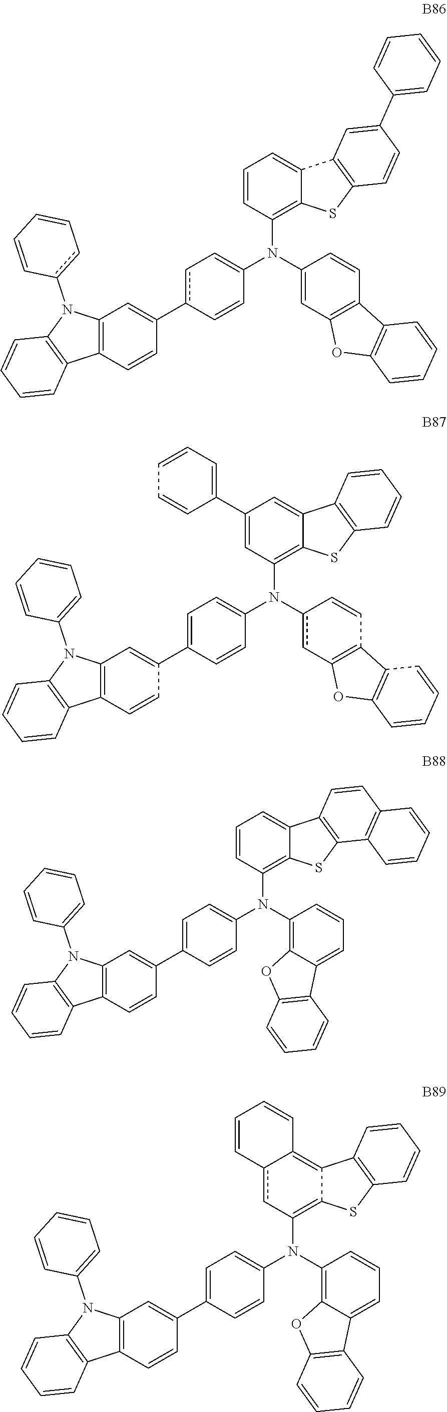

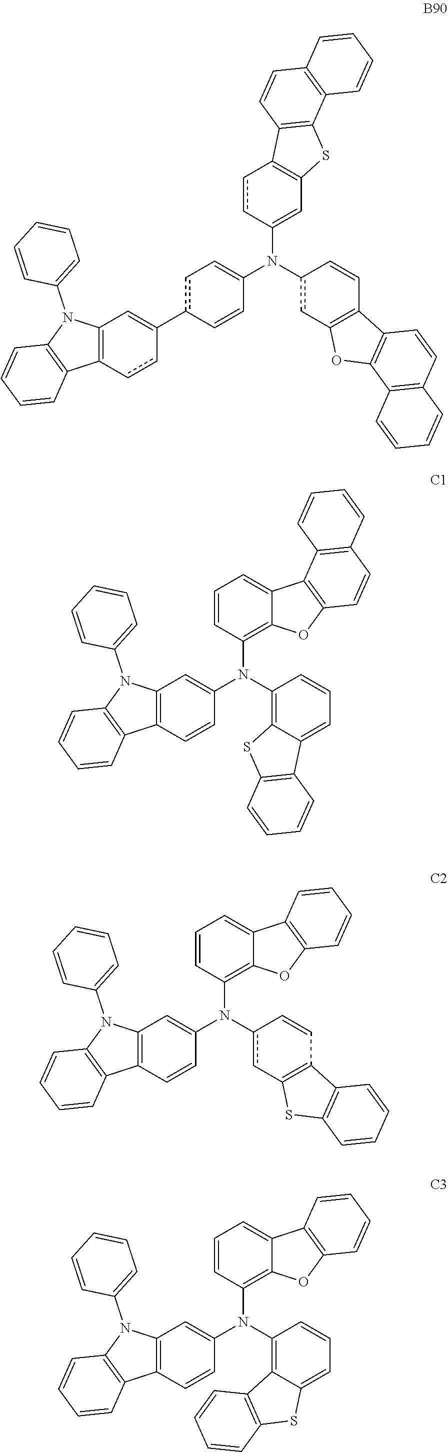

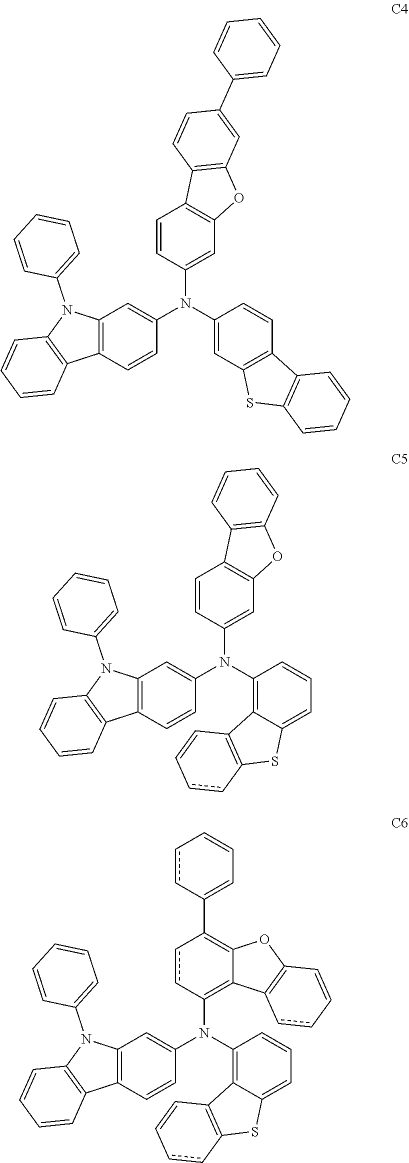

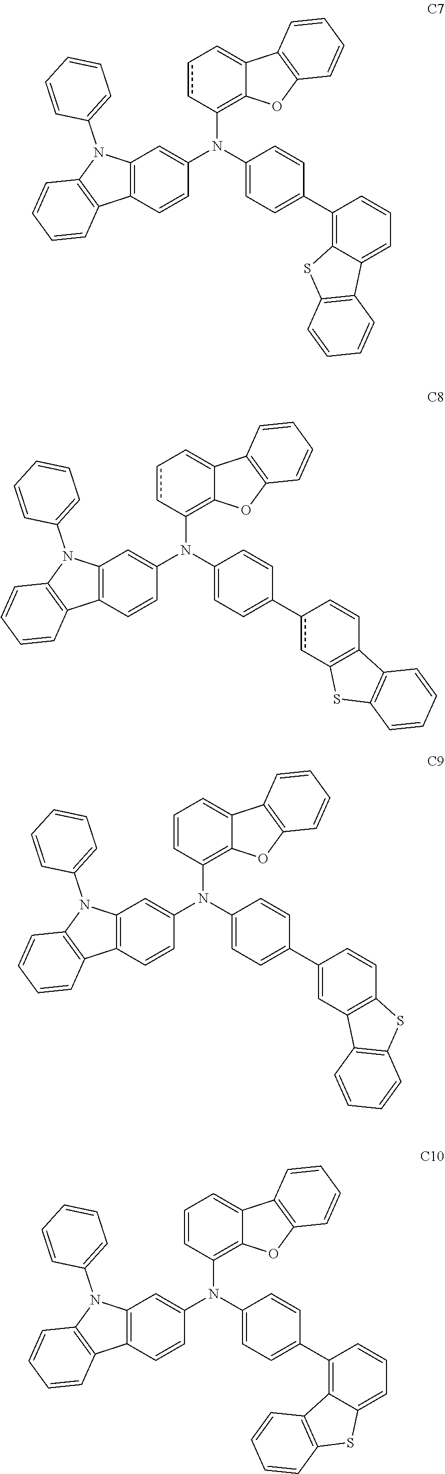

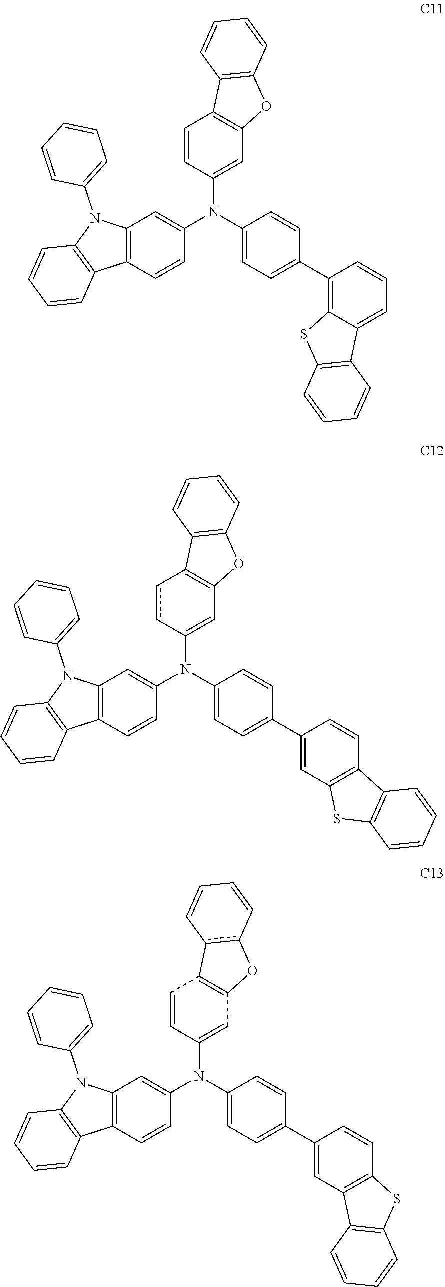

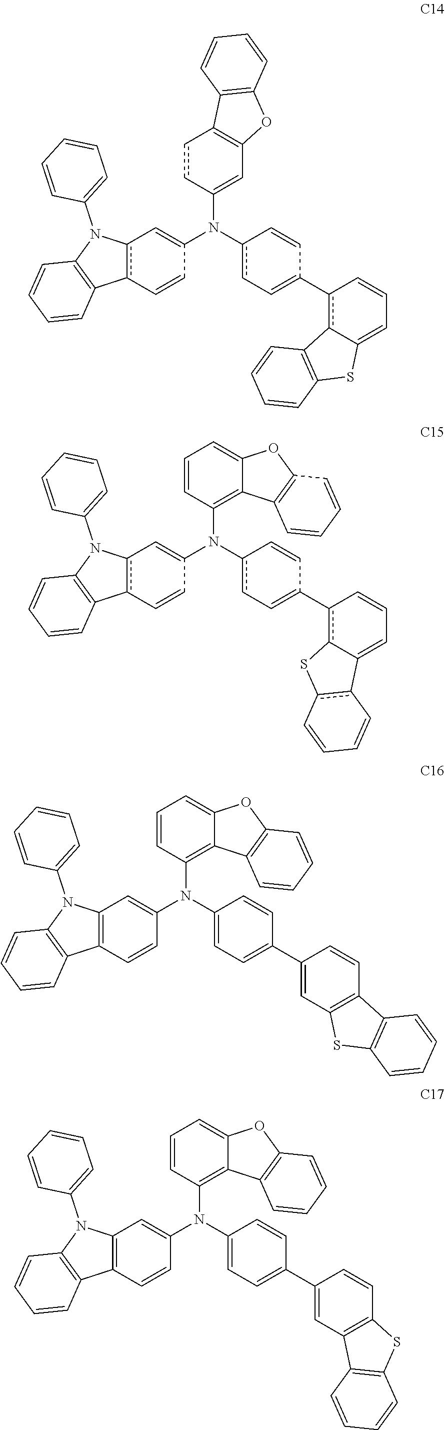

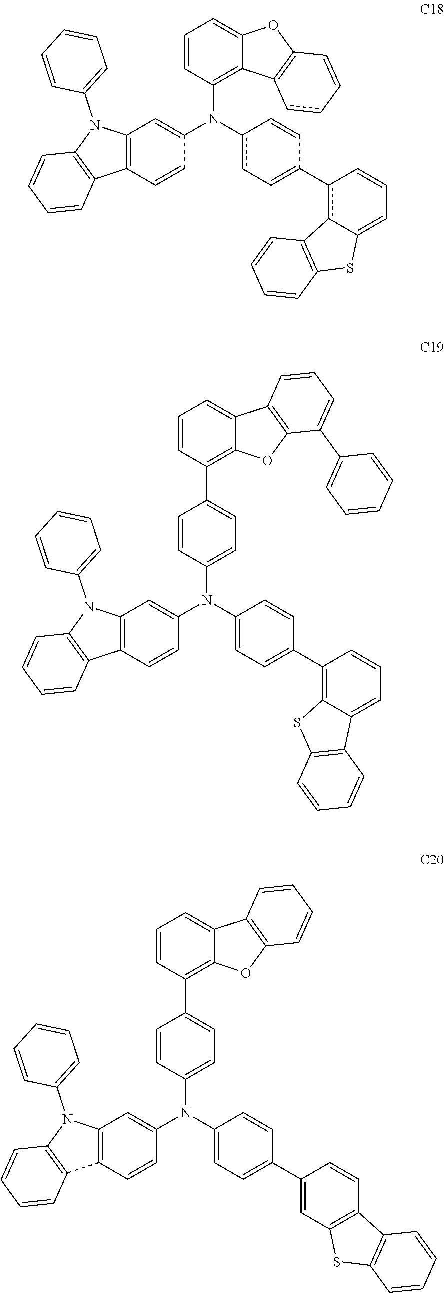

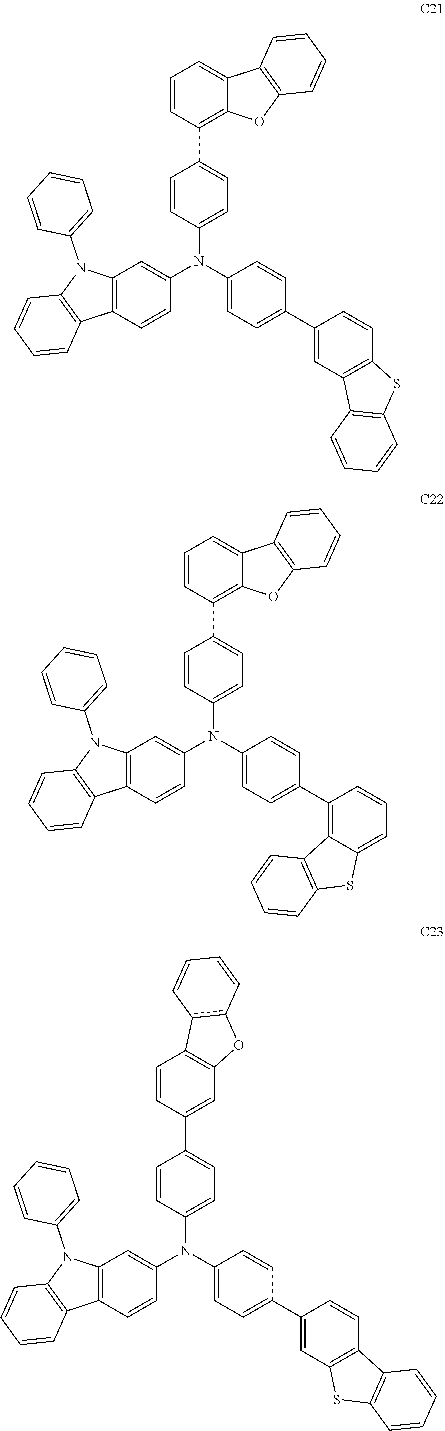

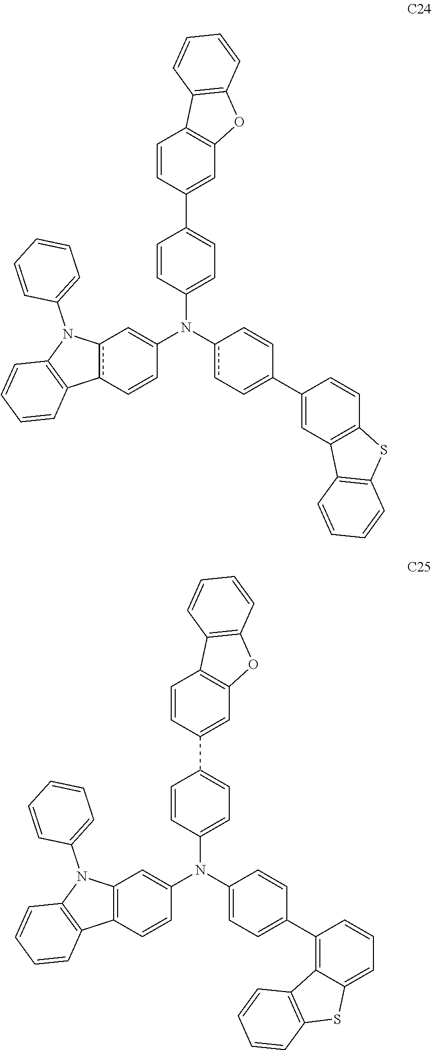

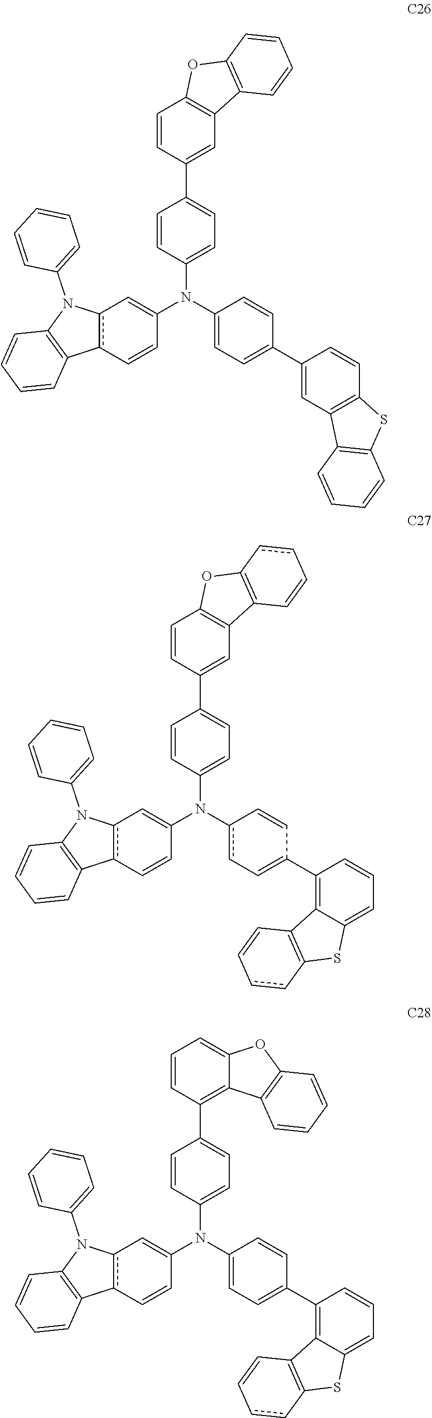

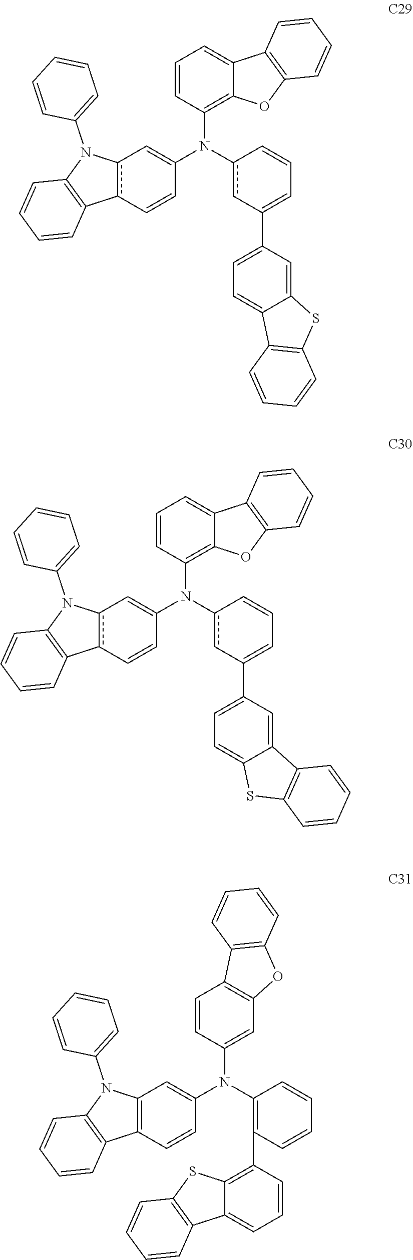

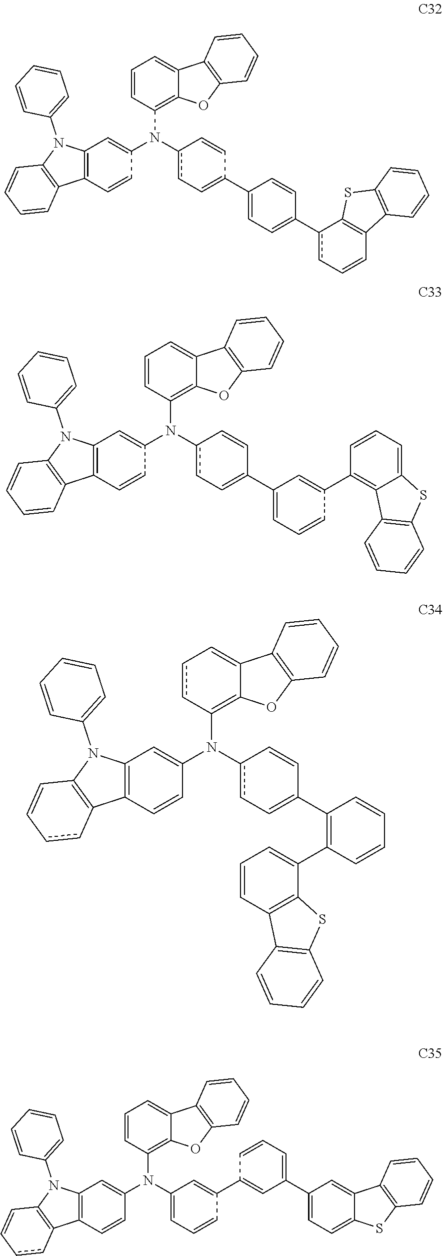

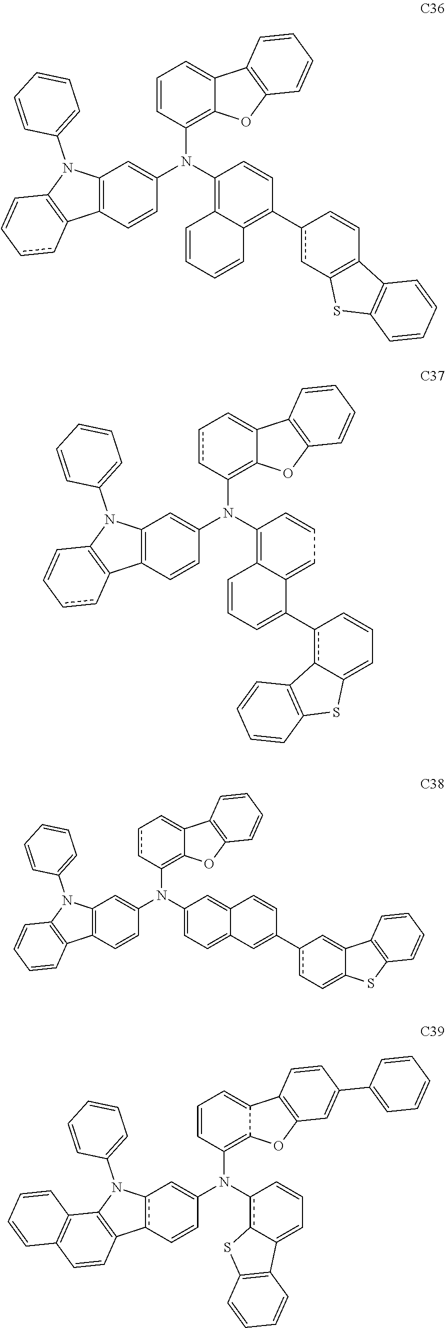

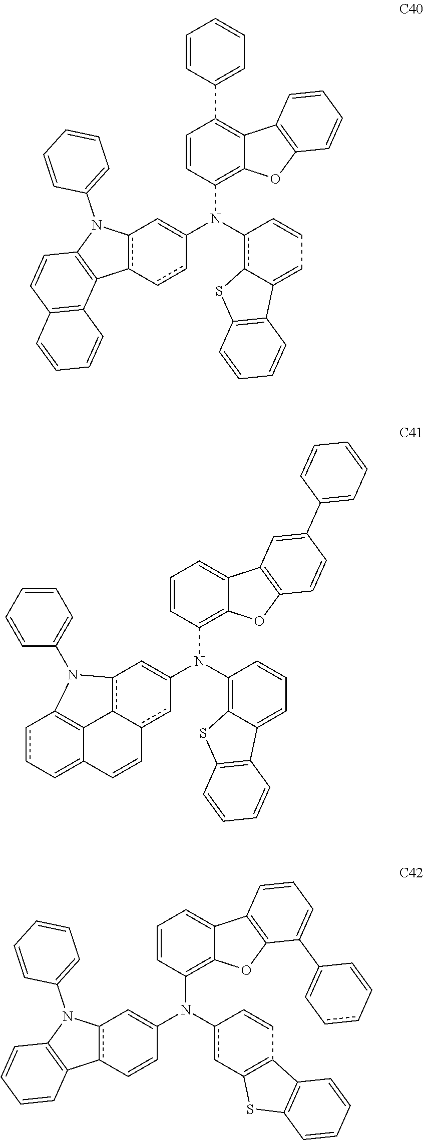

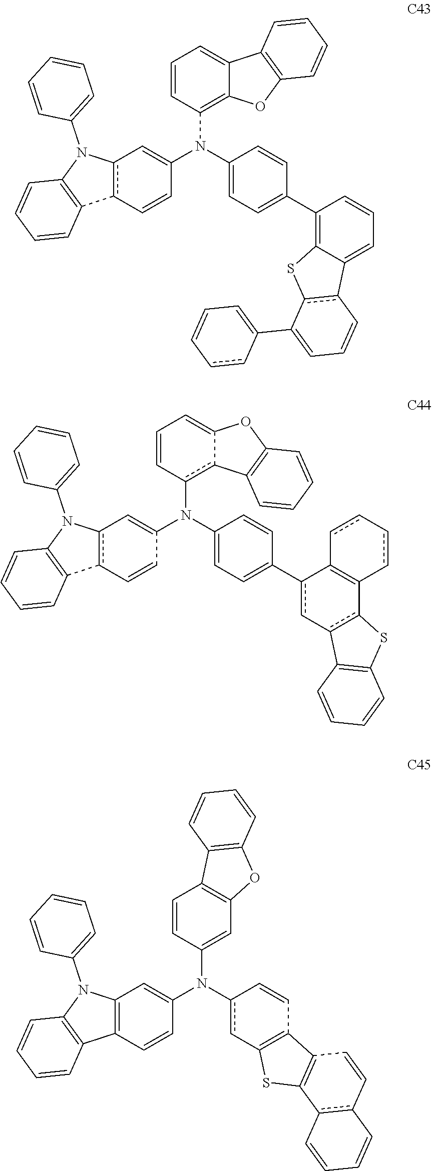

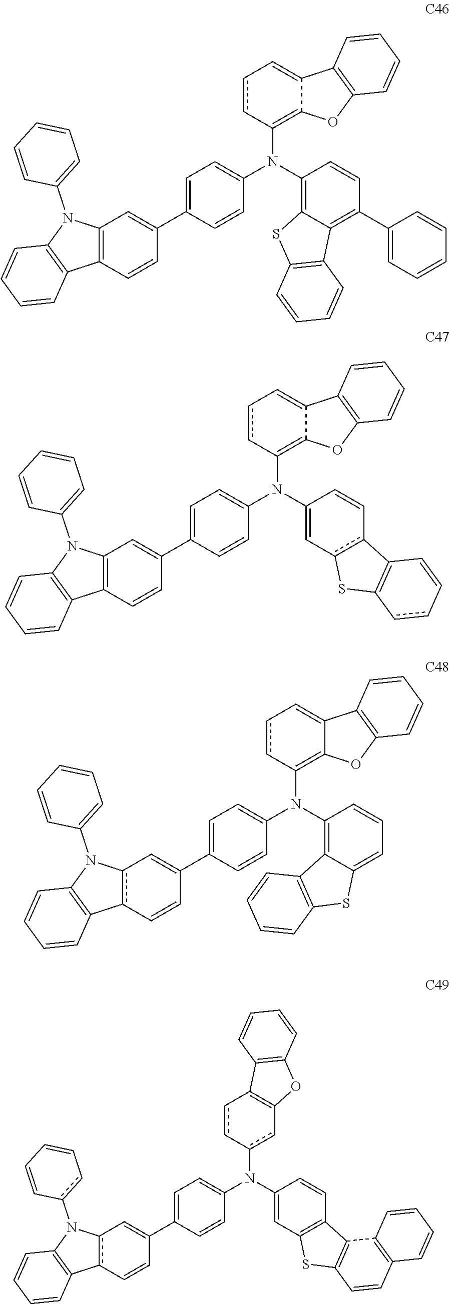

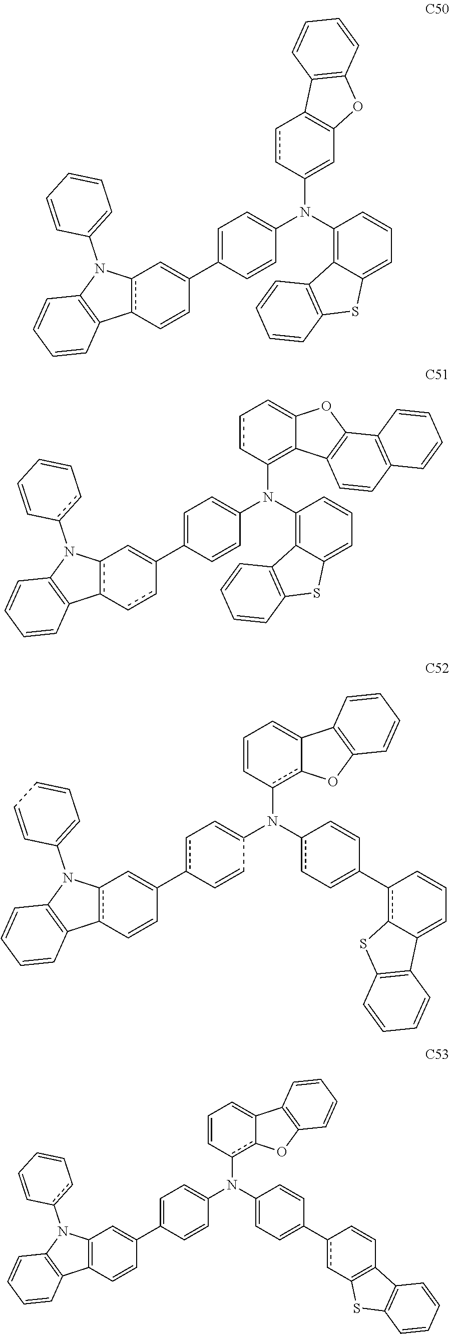

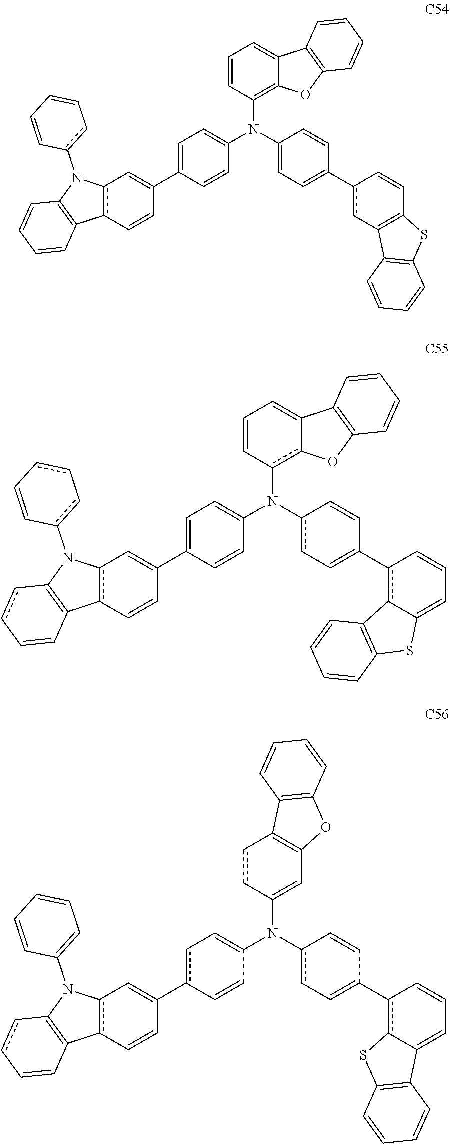

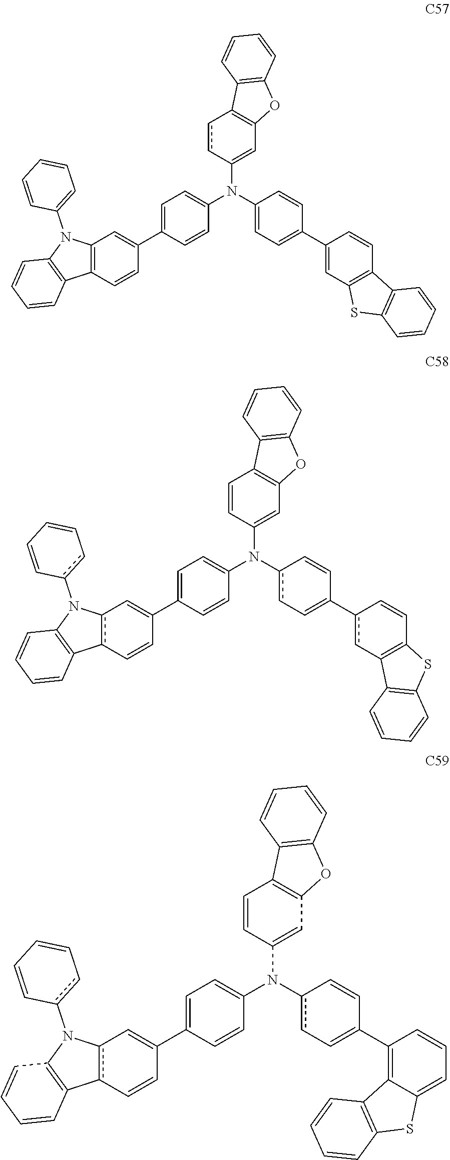

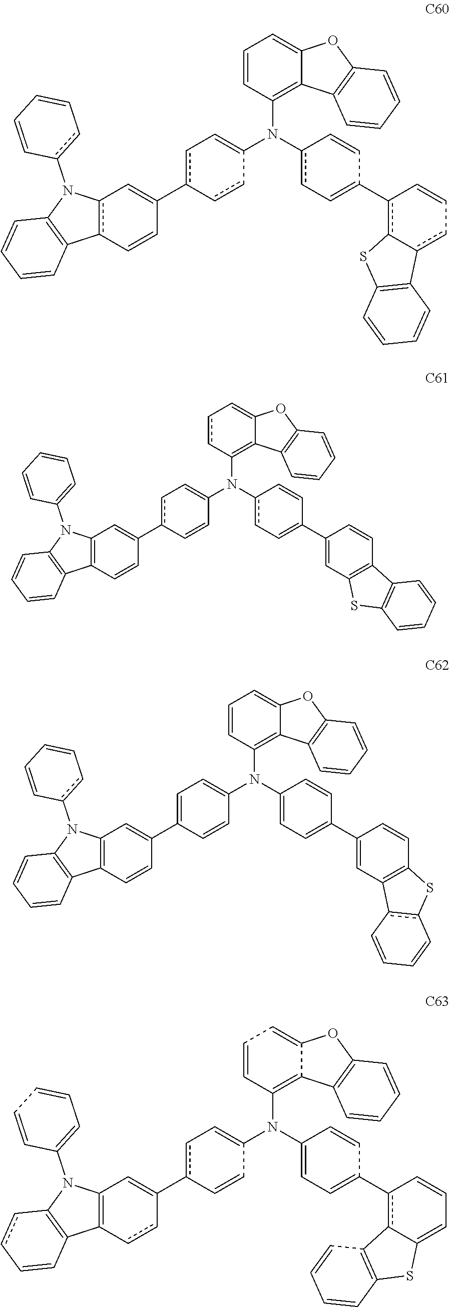

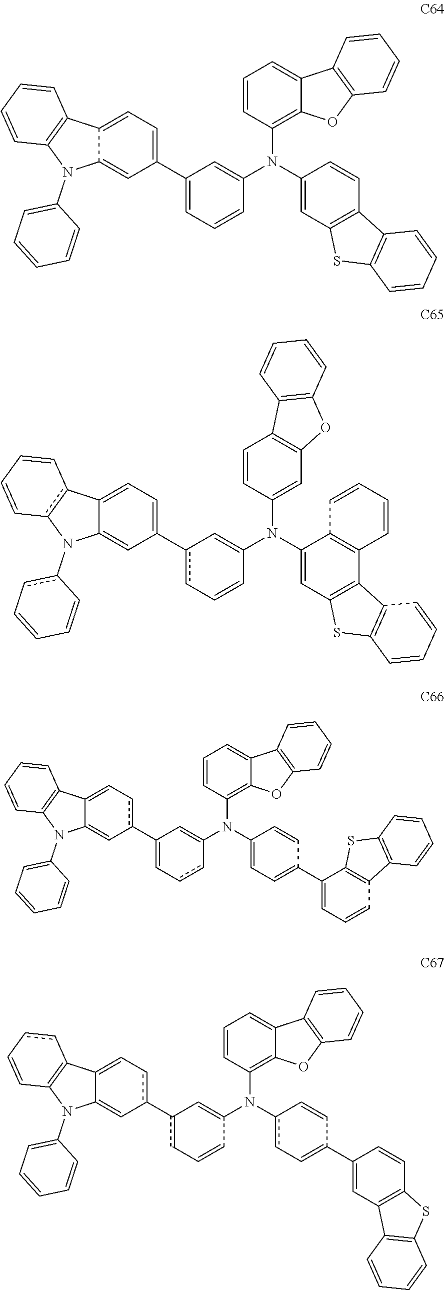

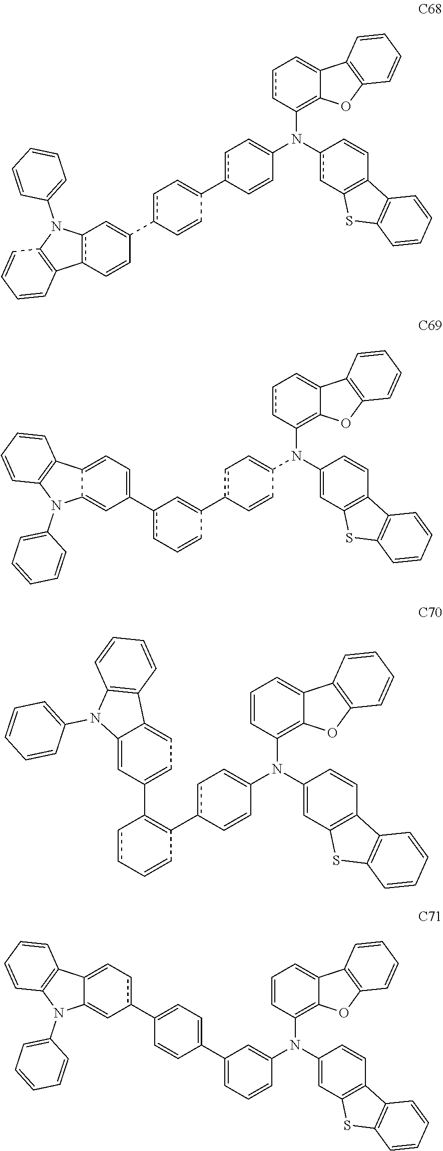

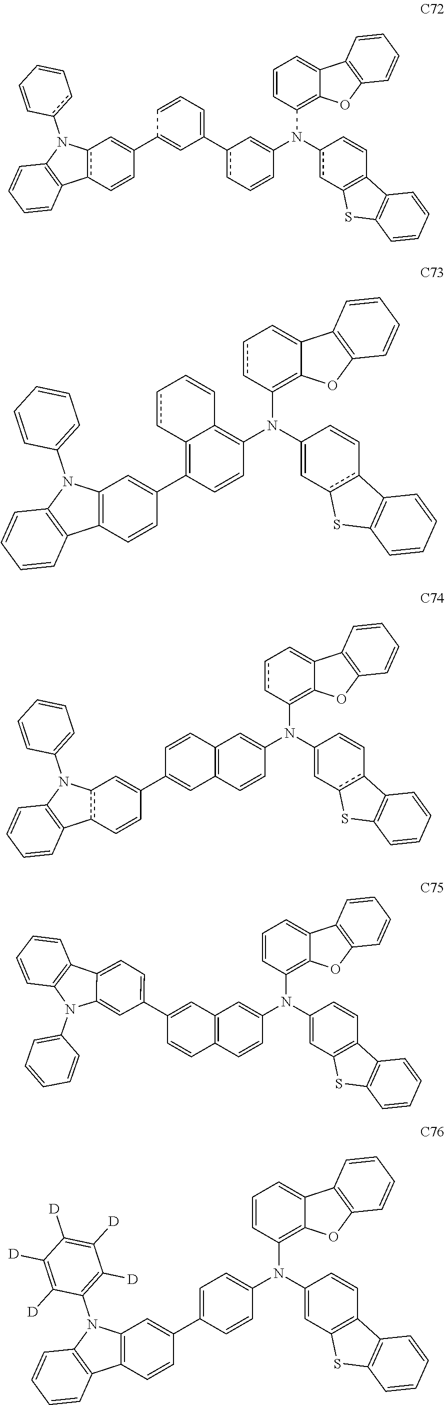

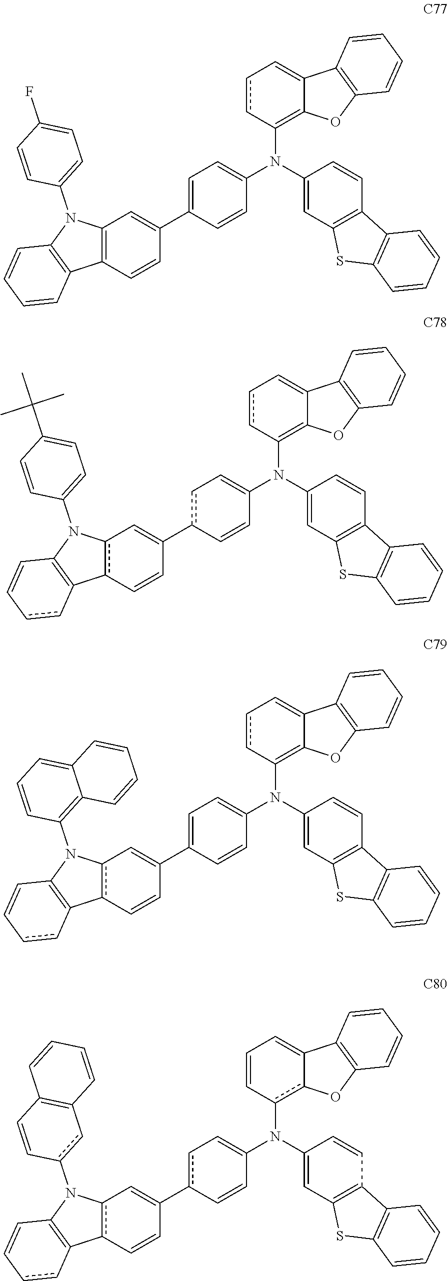

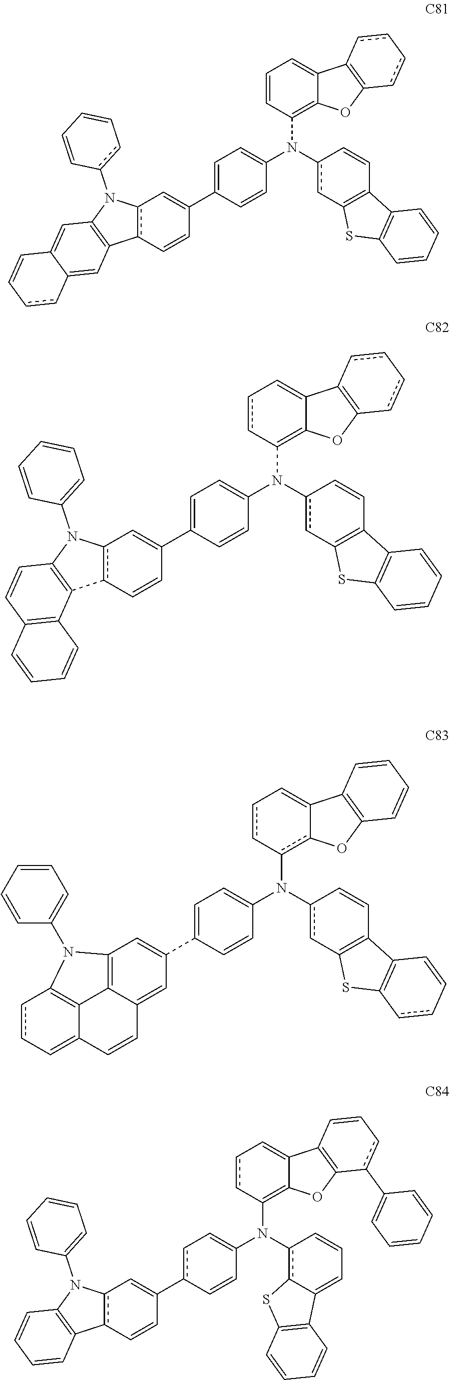

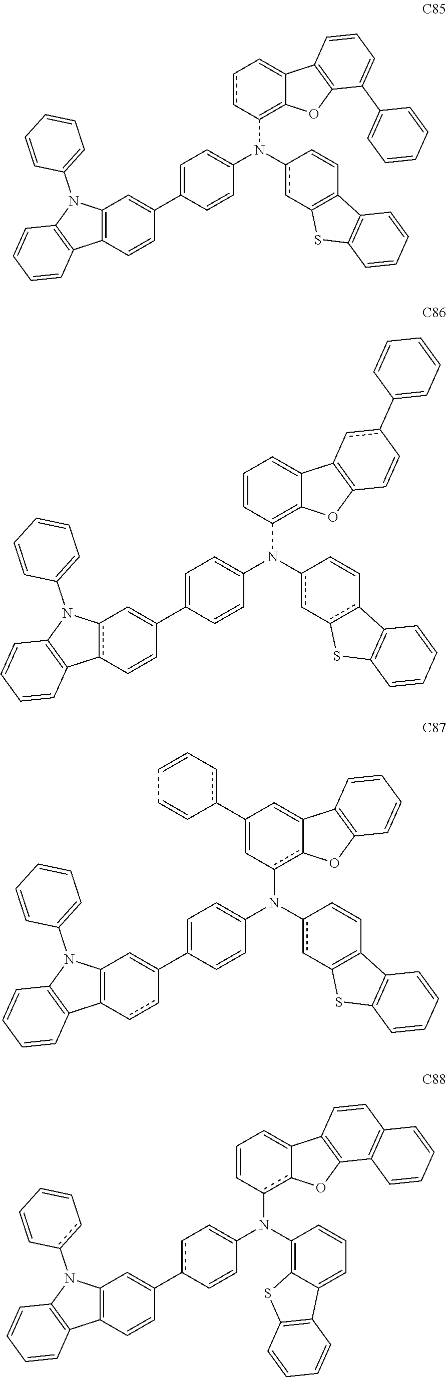

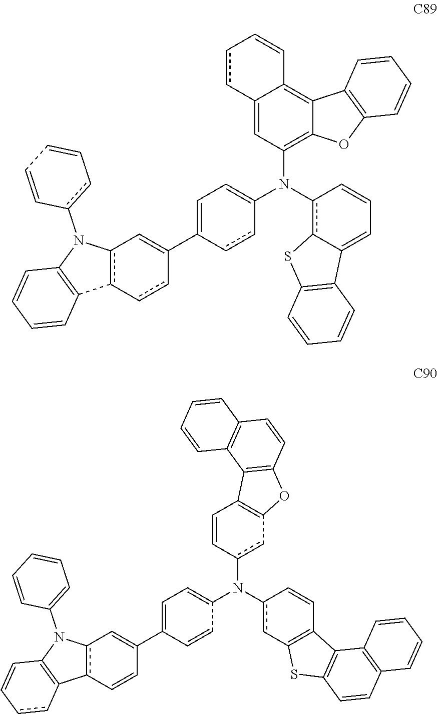

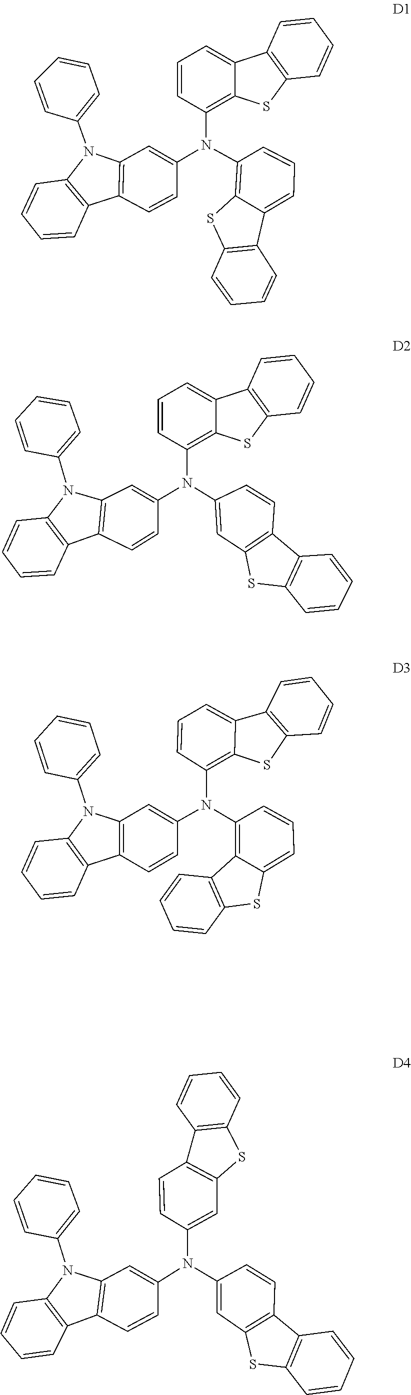

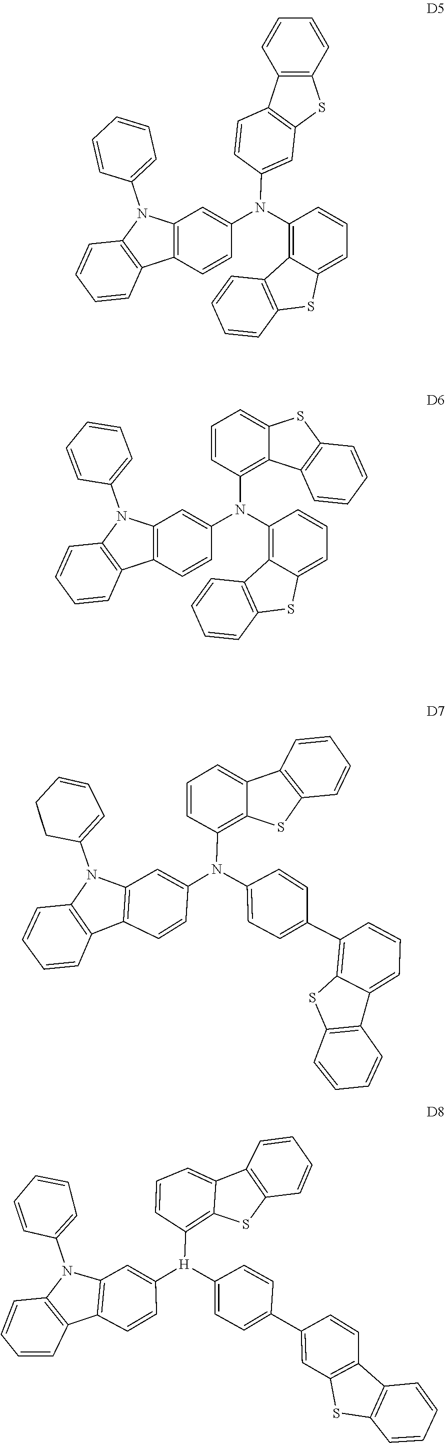

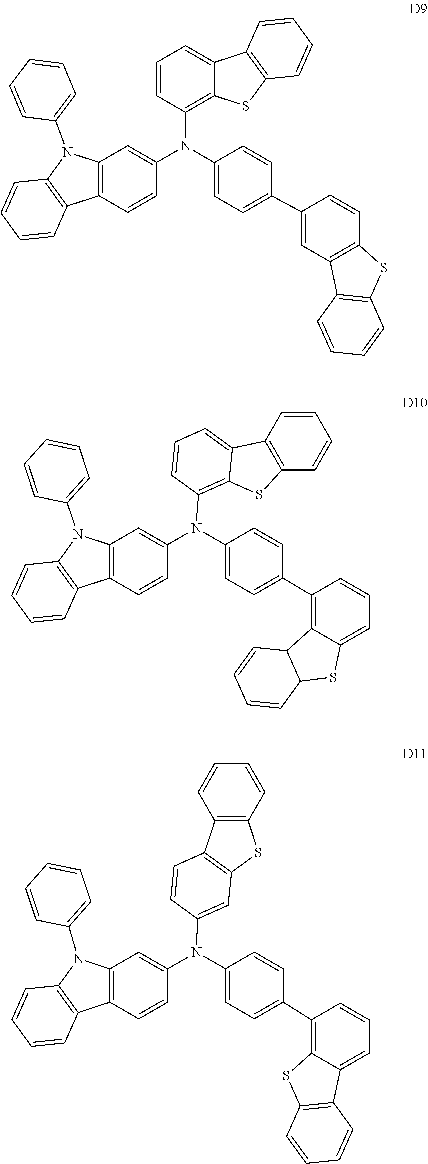 [SMILES: c1ccc(-n2c3ccccc3c3ccc(N(c4ccc(-c5ccc6sc7ccccc7c6c5)cc4)c4cccc5c4sc4ccccc45)cc32)cc1 ; C1=CC2Sc3cccc(-c4ccc(N(c5ccc6c7ccccc7n(-c7ccccc7)c6c5)c5cccc6c5sc5ccccc56)cc4)c3C2C=C1 ; c1ccc(-n2c3ccccc3c3ccc(N(c4ccc(-c5cccc6c5sc5ccccc56)cc4)c4ccc5c(c4)sc4ccccc45)cc32)cc1]

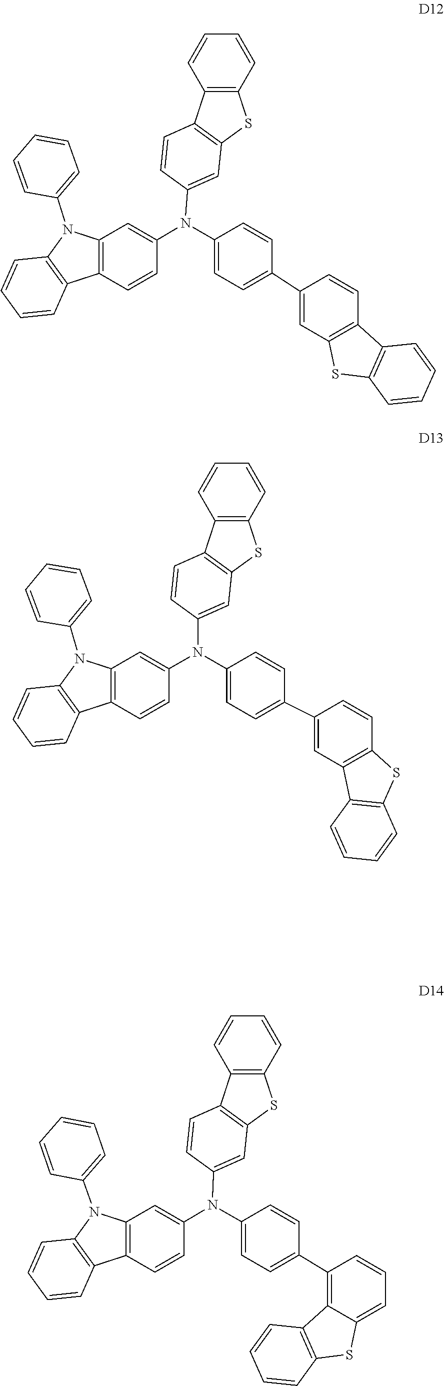 [SMILES: c1ccc(-n2c3ccccc3c3ccc(N(c4ccc(-c5ccc6c(c5)sc5ccccc56)cc4)c4ccc5c(c4)sc4ccccc45)cc32)cc1 ; c1ccc(-n2c3ccccc3c3ccc(N(c4ccc(-c5ccc6sc7ccccc7c6c5)cc4)c4ccc5c(c4)sc4ccccc45)cc32)cc1 ; c1ccc(-n2c3ccccc3c3ccc(N(c4ccc(-c5cccc6sc7ccccc7c56)cc4)c4ccc5c(c4)sc4ccccc45)cc32)cc1]

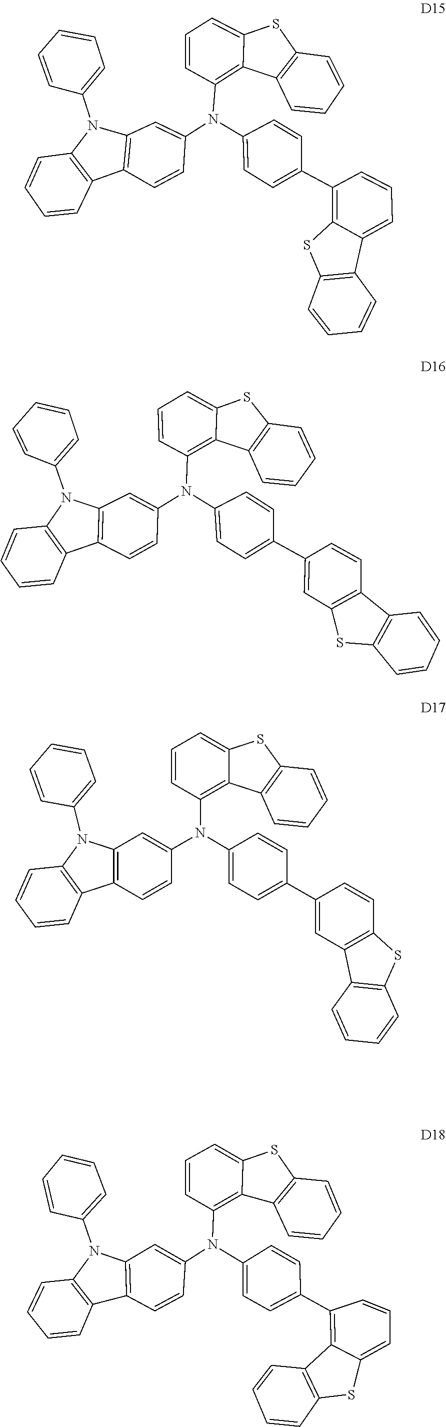 [SMILES: c1ccc(-n2c3ccccc3c3ccc(N(c4ccc(-c5cccc6c5Sc5ccccc5C6)cc4)c4cccc5sc6ccccc6c45)cc32)cc1 ; c1ccc(-n2c3ccccc3c3ccc(N(c4ccc(-c5ccc6c(c5)sc5ccccc56)cc4)c4cccc5sc6ccccc6c45)cc32)cc1 ; c1ccc(-n2c3ccccc3c3ccc(N(c4ccc(-c5ccc6sc7ccccc7c6c5)cc4)c4cccc5sc6ccccc6c45)cc32)cc1 ; c1ccc(-n2c3ccccc3c3ccc(N(c4ccc(-c5cccc6sc7ccccc7c56)cc4)c4cccc5sc6ccccc6c45)cc32)cc1]

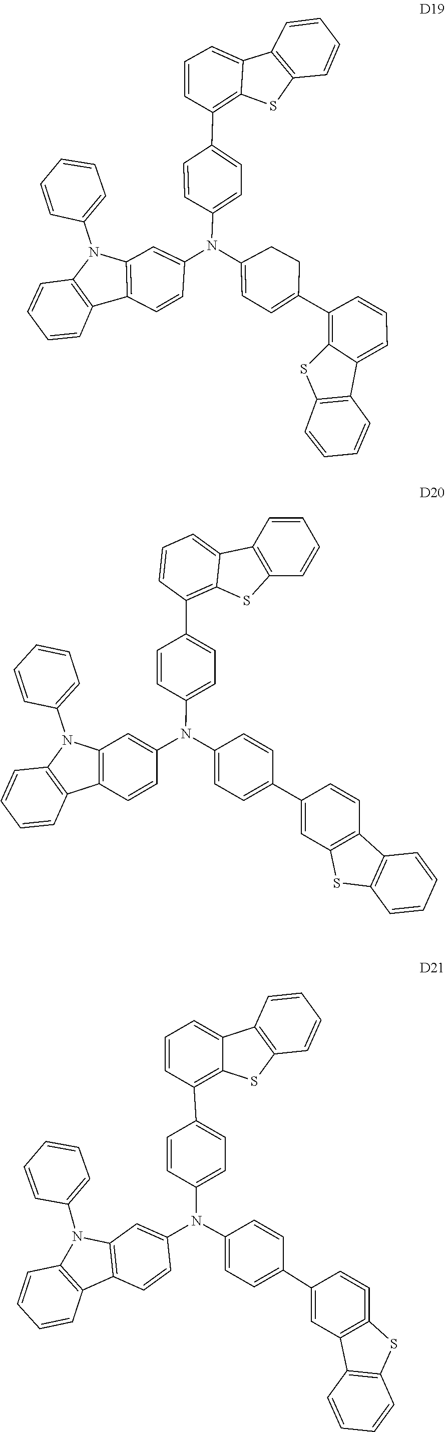

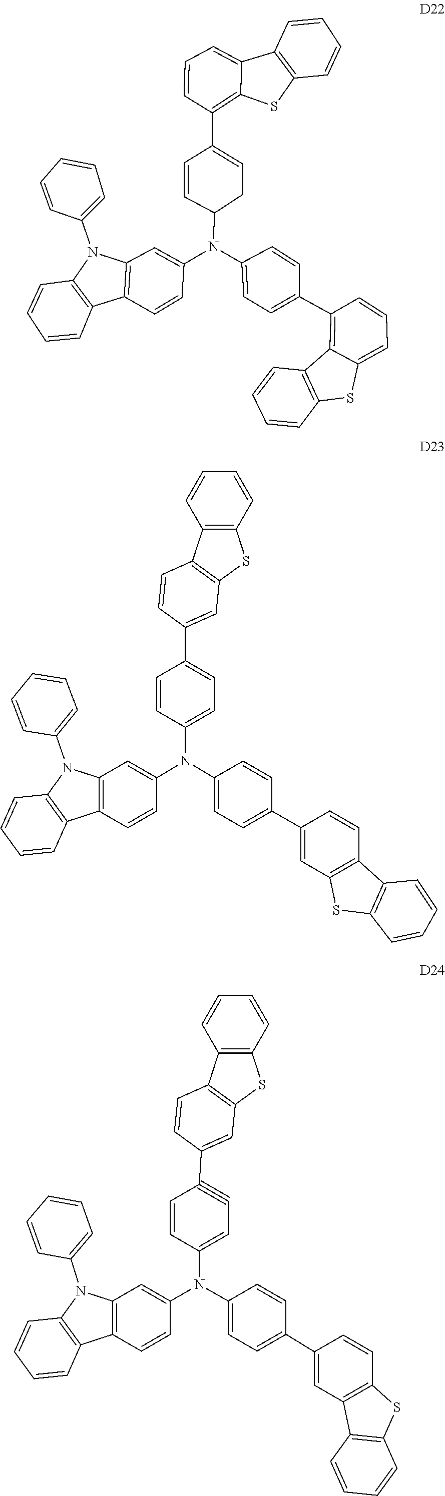

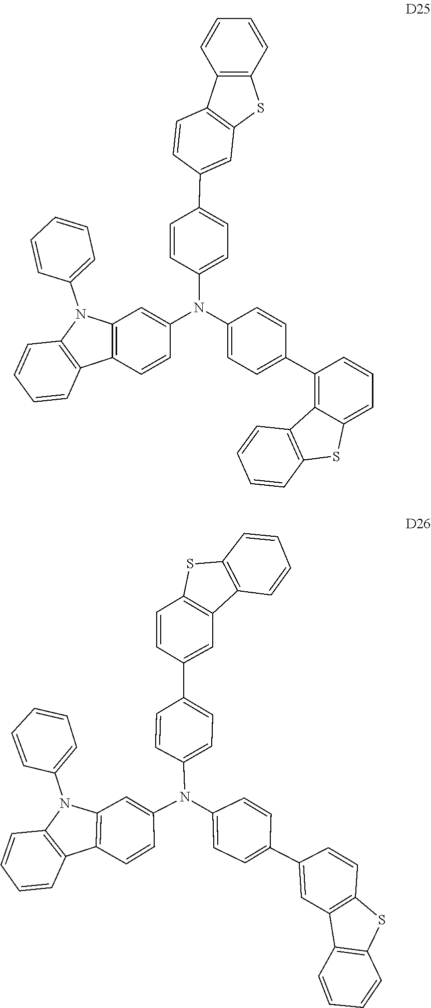 [SMILES: c1ccc(-n2c3ccccc3c3ccc(N(c4ccc(-c5ccc6c(c5)sc5ccccc56)cc4)c4ccc(-c5cccc6sc7ccccc7c56)cc4)cc32)cc1 ; c1ccc(-n2c3ccccc3c3ccc(N(c4ccc(-c5ccc6sc7ccccc7c6c5)cc4)c4ccc(-c5ccc6sc7ccccc7c6c5)cc4)cc32)cc1]

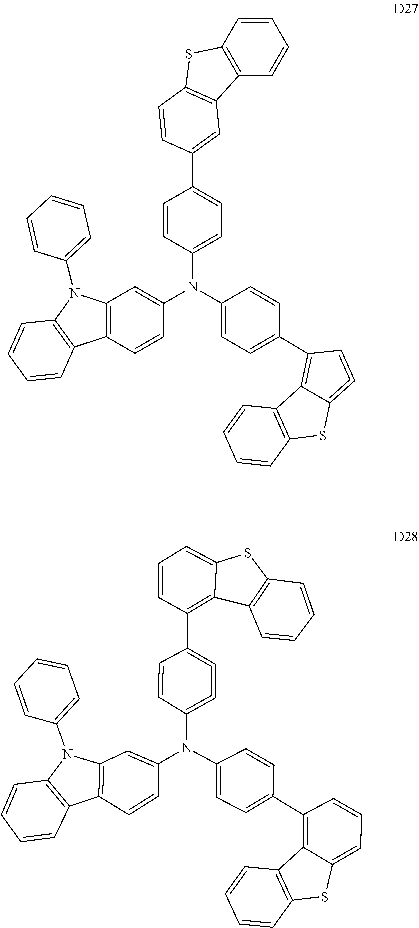 [SMILES: C1=CC(c2ccc(N(c3ccc(-c4ccc5sc6ccccc6c5c4)cc3)c3ccc4c5ccccc5n(-c5ccccc5)c4c3)cc2)=c2c=1sc1ccccc21 ; C1=C=C(N(c2ccc(-c3cccc4sc5ccccc5c34)cc2)c2ccc3c4ccccc4n(-c4ccccc4)c3c2)C=CC=1c1cccc2sc3ccccc3c12]

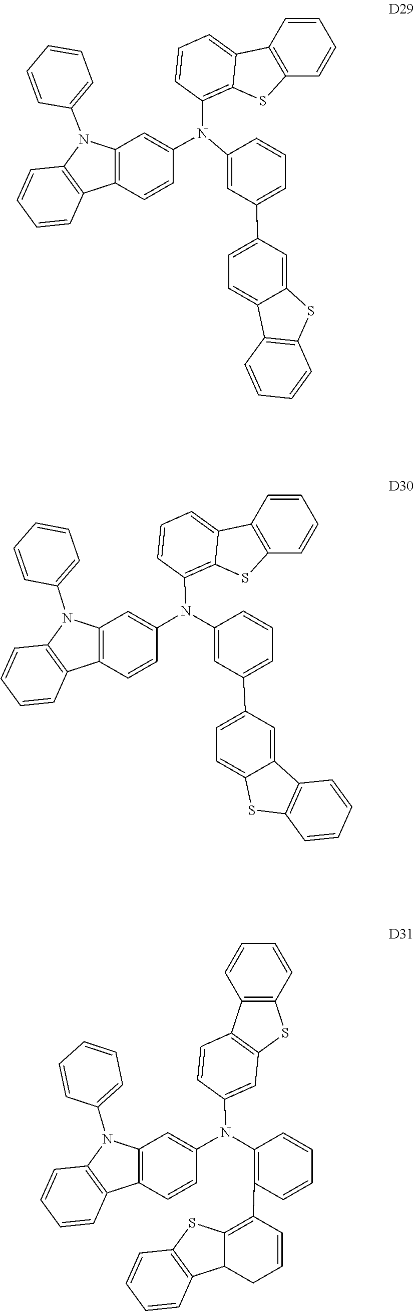 [SMILES: c1ccc(-n2c3ccccc3c3ccc(N(c4cccc(-c5ccc6c(c5)sc5ccccc56)c4)c4cccc5c4sc4ccccc45)cc32)cc1 ; c1ccc(-n2c3ccccc3c3ccc(N(c4cccc(-c5ccc6sc7ccccc7c6c5)c4)c4cccc5c4sc4ccccc45)cc32)cc1 ; C1=CC(c2ccccc2N(c2ccc3c(c2)sc2ccccc23)c2ccc3c4ccccc4n(-c4ccccc4)c3c2)=C2Sc3ccccc3C2C1]

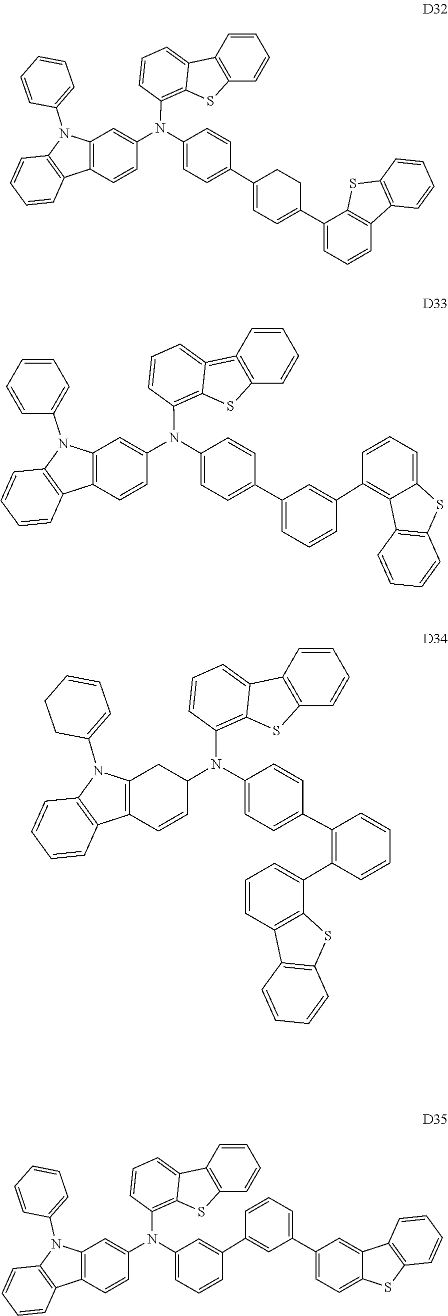

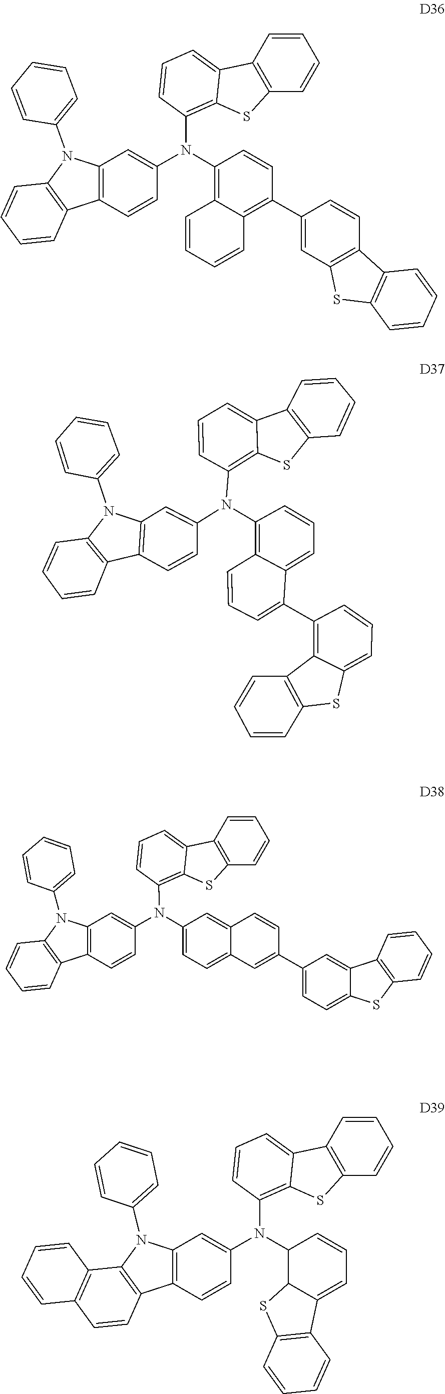 [SMILES: c1ccc(-n2c3ccccc3c3ccc(N(c4ccc(-c5ccc6c(c5)sc5ccccc56)c5ccccc45)c4cccc5c4sc4ccccc45)cc32)cc1 ; c1ccc(-n2c3ccccc3c3ccc(N(c4cccc5c(-c6cccc7sc8ccccc8c67)cccc45)c4cccc5c4sc4ccccc45)cc32)cc1 ; c1ccc(-n2c3ccccc3c3ccc(N(c4ccc5cc(-c6ccc7sc8ccccc8c7c6)ccc5c4)c4cccc5c4sc4ccccc45)cc32)cc1 ; C1=CC(N(c2ccc3c4ccc5ccccc5c4n(-c4ccccc4)c3c2)c2cccc3c2sc2ccccc23)C2Sc3ccccc3C2=C1]

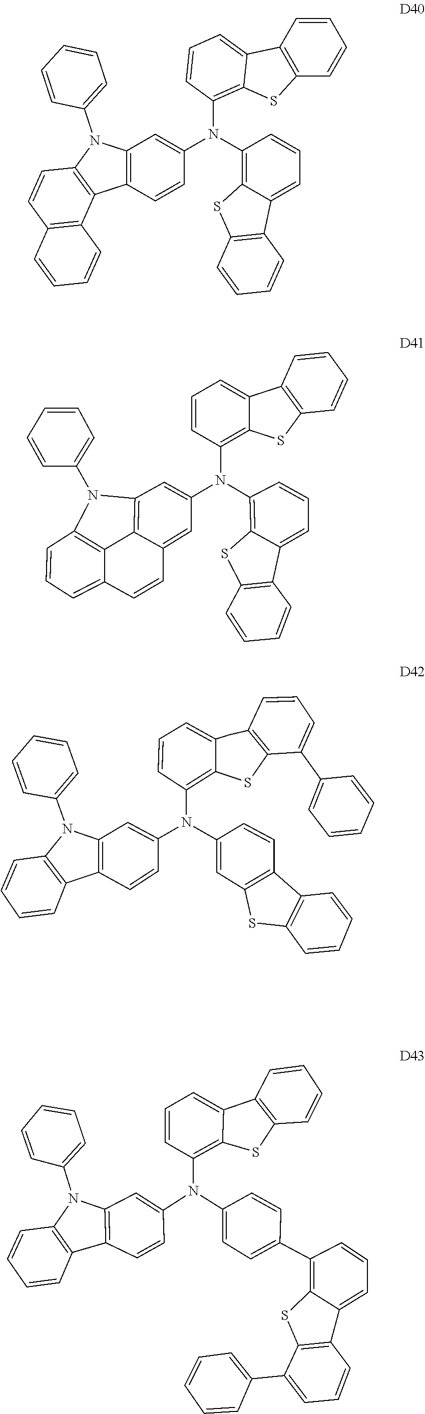

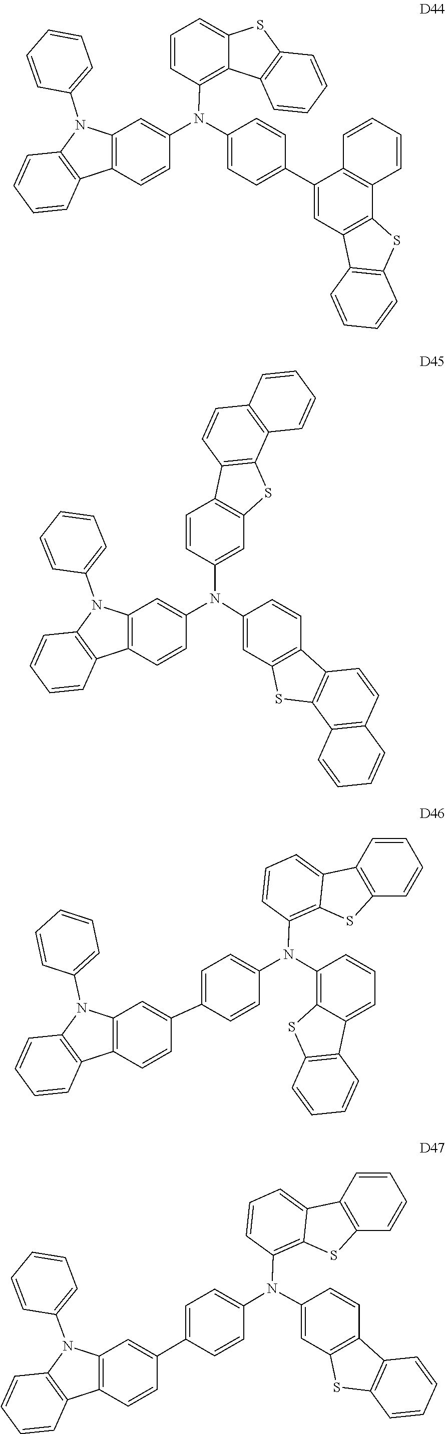 [SMILES: c1ccc(-n2c3ccccc3c3ccc(N(c4ccc(-c5cc6c7ccccc7sc6c6ccccc56)cc4)c4cccc5sc6ccccc6c45)cc32)cc1 ; c1ccc(-n2c3ccccc3c3ccc(N(c4ccc5c(c4)sc4c6ccccc6ccc54)c4ccc5c(c4)sc4c6ccccc6ccc54)cc32)cc1 ; c1ccc(-n2c3ccccc3c3ccc(-c4ccc(N(c5cccc6c5sc5ccccc56)c5cccc6c5sc5ccccc56)cc4)cc32)cc1 ; c1ccc(-n2c3ccccc3c3ccc(-c4ccc(N(c5ccc6c(c5)sc5ccccc56)c5cccc6c5sc5ccccc56)cc4)cc32)cc1]

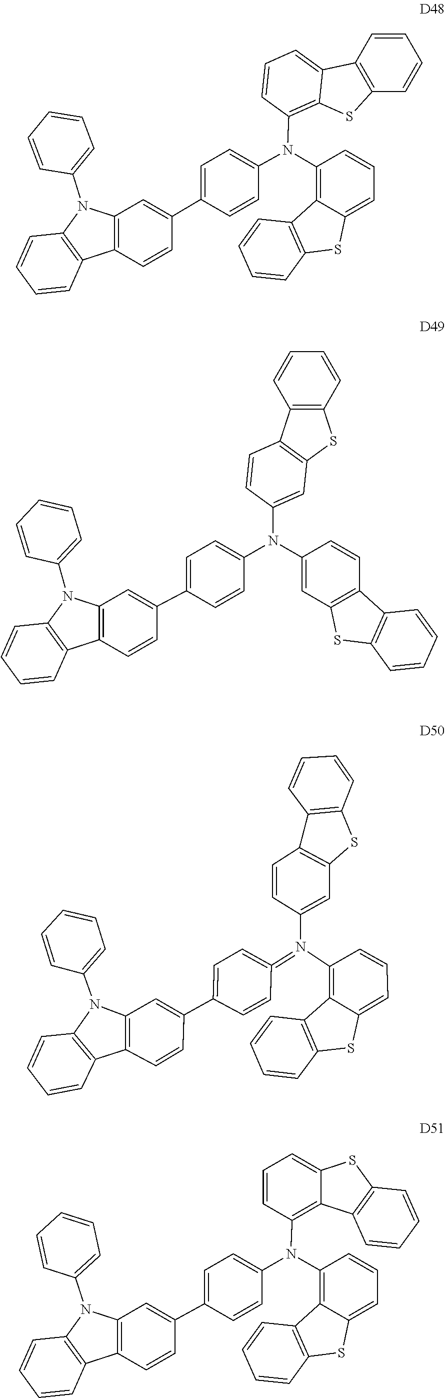

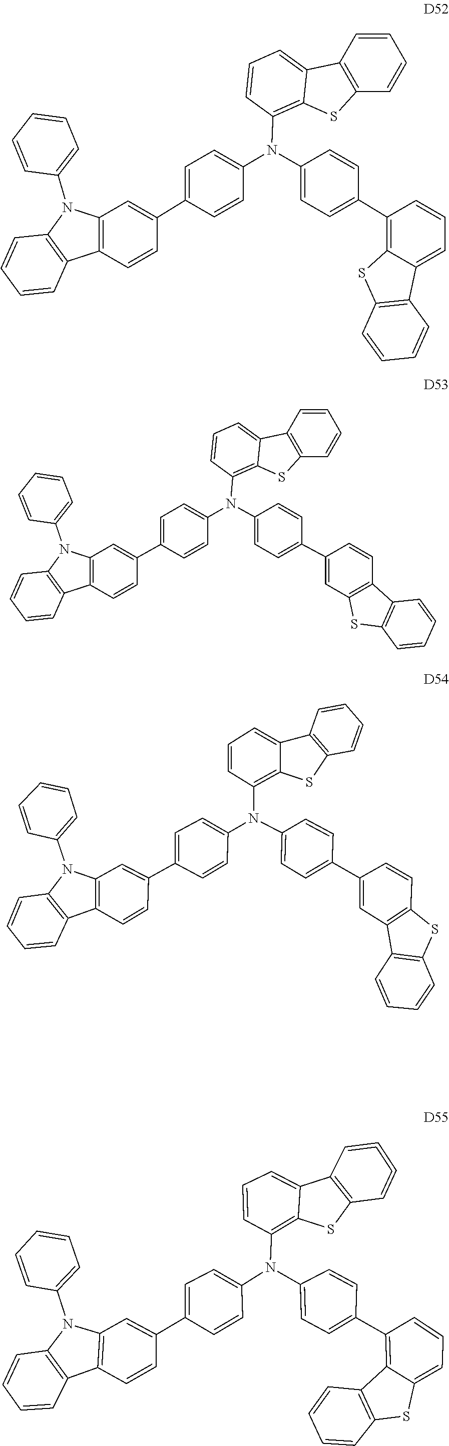 [SMILES: c1ccc(-n2c3ccccc3c3ccc(-c4ccc(N(c5ccc(-c6cccc7c6sc6ccccc67)cc5)c5cccc6c5sc5ccccc56)cc4)cc32)cc1 ; c1ccc(-n2c3ccccc3c3ccc(-c4ccc(N(c5ccc(-c6ccc7c(c6)sc6ccccc67)cc5)c5cccc6c5sc5ccccc56)cc4)cc32)cc1 ; c1ccc(-n2c3ccccc3c3ccc(-c4ccc(N(c5ccc(-c6ccc7sc8ccccc8c7c6)cc5)c5cccc6c5sc5ccccc56)cc4)cc32)cc1 ; C1=C=Cc2c(sc3c(N(c4ccc(-c5ccc6c7ccccc7n(-c7ccccc7)c6c5)cc4)c4ccc(-c5cccc6sc7ccccc7c56)cc4)cccc23)C=1]

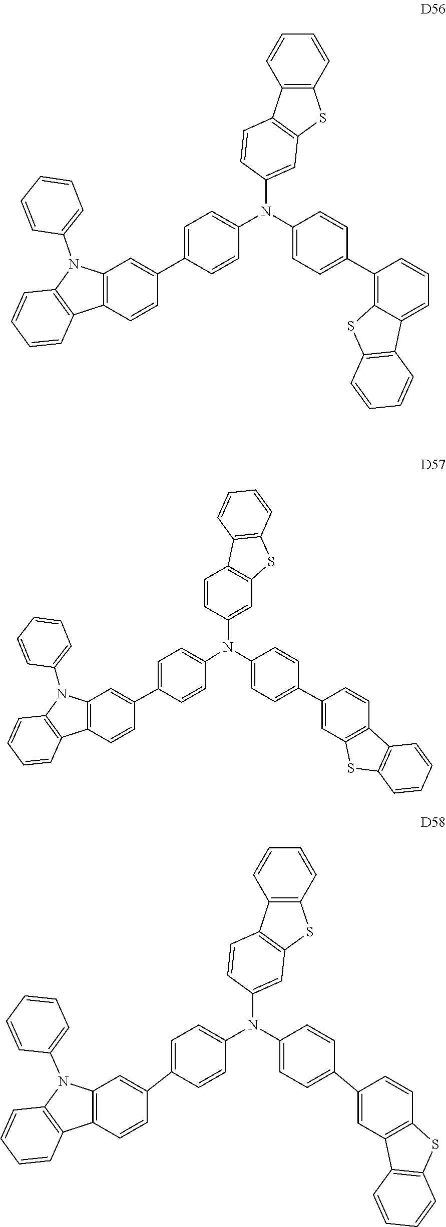 [SMILES: c1ccc(-n2c3ccccc3c3ccc(-c4ccc(N(c5ccc(-c6cccc7c6sc6ccccc67)cc5)c5ccc6c(c5)sc5ccccc56)cc4)cc32)cc1 ; c1ccc(-n2c3ccccc3c3ccc(-c4ccc(N(c5ccc(-c6ccc7c(c6)sc6ccccc67)cc5)c5ccc6c(c5)sc5ccccc56)cc4)cc32)cc1 ; c1ccc(-n2c3ccccc3c3ccc(-c4ccc(N(c5ccc(-c6ccc7sc8ccccc8c7c6)cc5)c5ccc6c(c5)sc5ccccc56)cc4)cc32)cc1]

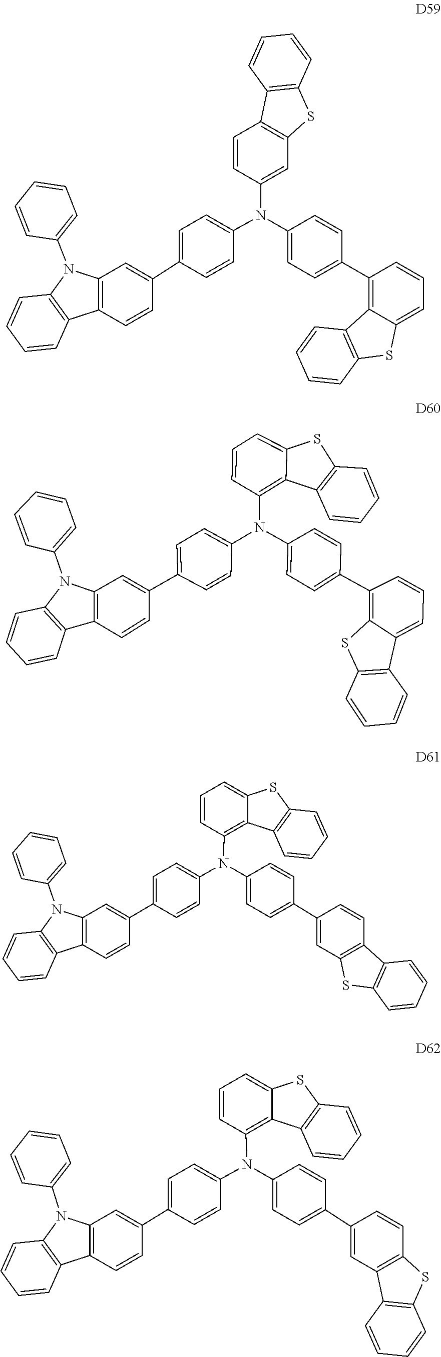 [SMILES: c1ccc(-n2c3ccccc3c3ccc(-c4ccc(N(c5ccc(-c6cccc7sc8ccccc8c67)cc5)c5ccc6c(c5)sc5ccccc56)cc4)cc32)cc1 ; c1ccc(-n2c3ccccc3c3ccc(-c4ccc(N(c5ccc(-c6cccc7c6sc6ccccc67)cc5)c5cccc6sc7ccccc7c56)cc4)cc32)cc1 ; c1ccc(-n2c3ccccc3c3ccc(-c4ccc(N(c5ccc(-c6ccc7c(c6)sc6ccccc67)cc5)c5cccc6sc7ccccc7c56)cc4)cc32)cc1 ; c1ccc(-n2c3ccccc3c3ccc(-c4ccc(N(c5ccc(-c6ccc7sc8ccccc8c7c6)cc5)c5cccc6sc7ccccc7c56)cc4)cc32)cc1]

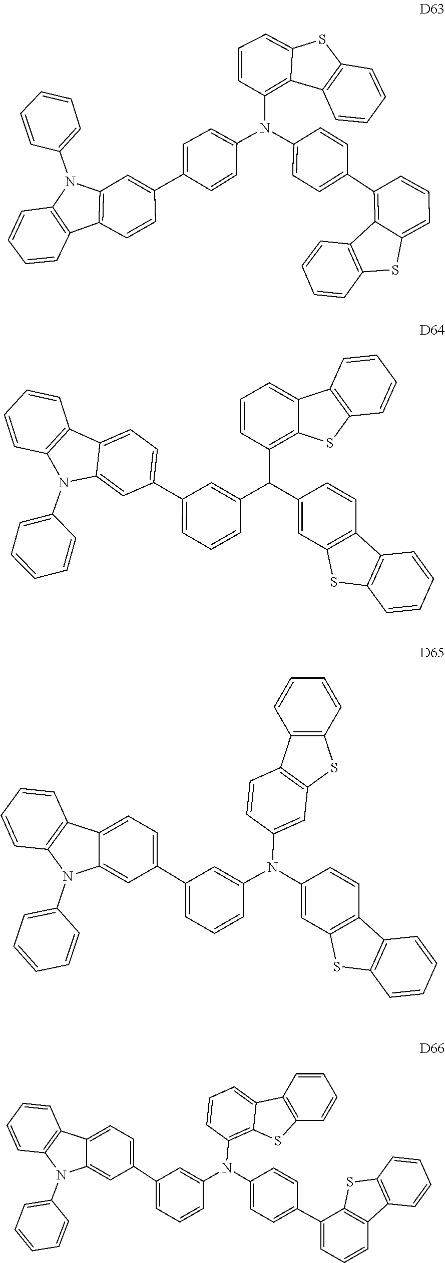 [SMILES: c1ccc(-n2c3ccccc3c3ccc(-c4ccc(N(c5ccc(-c6cccc7sc8ccccc8c67)cc5)c5cccc6sc7ccccc7c56)cc4)cc32)cc1 ; c1ccc(-n2c3ccccc3c3ccc(-c4cccc(C(c5ccc6c(c5)sc5ccccc56)c5cccc6c5sc5ccccc56)c4)cc32)cc1 ; c1ccc(-n2c3ccccc3c3ccc(-c4cccc(N(c5ccc6c(c5)sc5ccccc56)c5ccc6c(c5)sc5ccccc56)c4)cc32)cc1 ; c1ccc(-n2c3ccccc3c3ccc(-c4cccc(N(c5ccc(-c6cccc7c6sc6ccccc67)cc5)c5cccc6c5sc5ccccc56)c4)cc32)cc1]

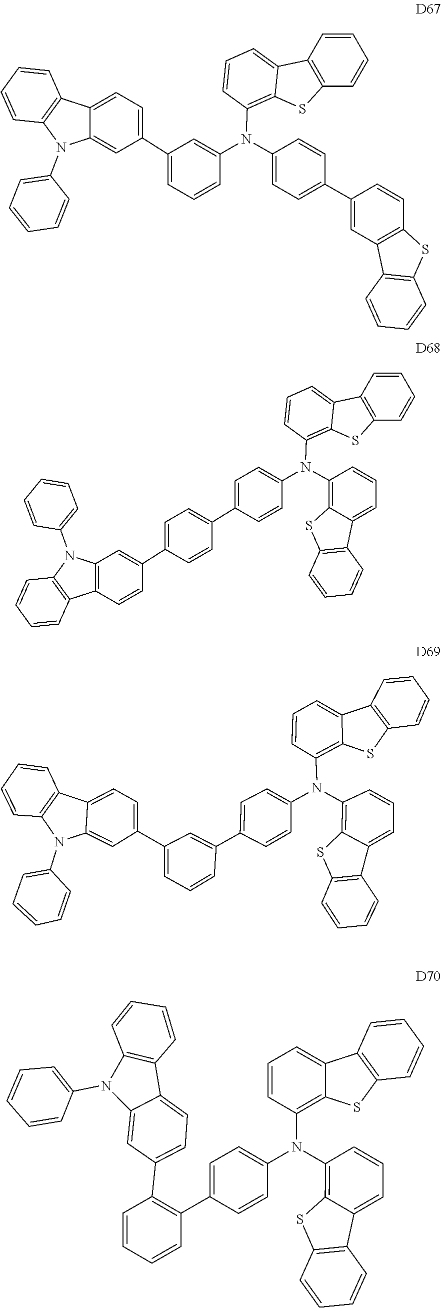 [SMILES: c1ccc(-n2c3ccccc3c3ccc(-c4cccc(N(c5ccc(-c6ccc7sc8ccccc8c7c6)cc5)c5cccc6c5sc5ccccc56)c4)cc32)cc1 ; c1ccc(-n2c3ccccc3c3ccc(-c4ccc(-c5ccc(N(c6cccc7c6sc6ccccc67)c6cccc7c6sc6ccccc67)cc5)cc4)cc32)cc1 ; c1ccc(-n2c3ccccc3c3ccc(-c4cccc(-c5ccc(N(c6cccc7c6sc6ccccc67)c6cccc7c6sc6ccccc67)cc5)c4)cc32)cc1 ; c1ccc(-n2c3ccccc3c3ccc(-c4ccccc4-c4ccc(N(c5cccc6c5sc5ccccc56)c5cccc6c5sc5ccccc56)cc4)cc32)cc1]

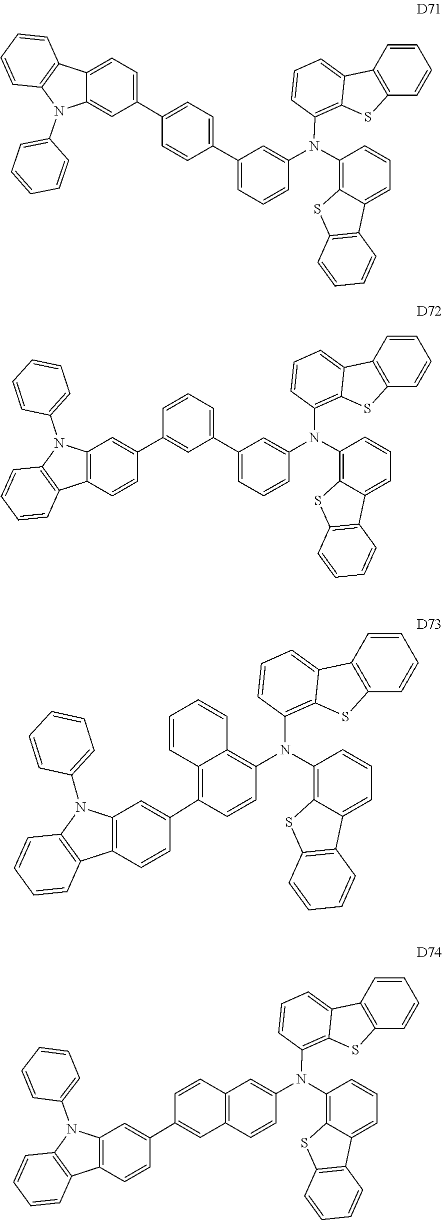

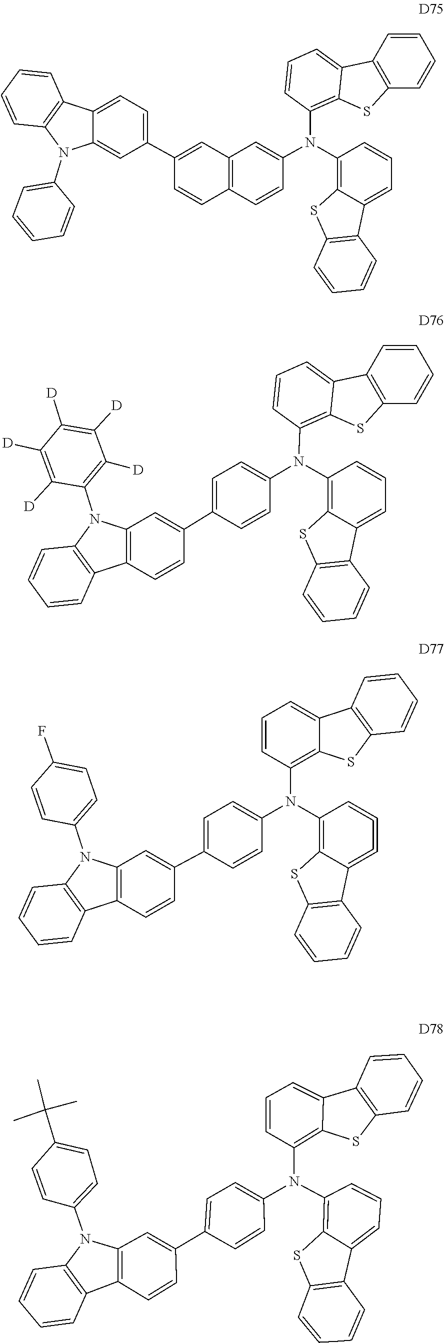 [SMILES: c1ccc(-n2c3ccccc3c3ccc(-c4ccc5ccc(N(c6cccc7c6sc6ccccc67)c6cccc7c6sc6ccccc67)cc5c4)cc32)cc1 ; [2H]c1c([2H])c([2H])c(-n2c3ccccc3c3ccc(-c4ccc(N(c5cccc6c5sc5ccccc56)c5cccc6c5sc5ccccc56)cc4)cc32)c([2H])c1[2H] ; Fc1ccc(-n2c3ccccc3c3ccc(-c4ccc(N(C5=c6sc7ccccc7c6=C=C=C5)c5cccc6c5sc5ccccc56)cc4)cc32)cc1 ; CC(C)(C)c1ccc(-n2c3ccccc3c3ccc(-c4ccc(N(c5cccc6c7c(sc56)C=CCC=C7)c5cccc6c5sc5ccccc56)cc4)cc32)cc1]

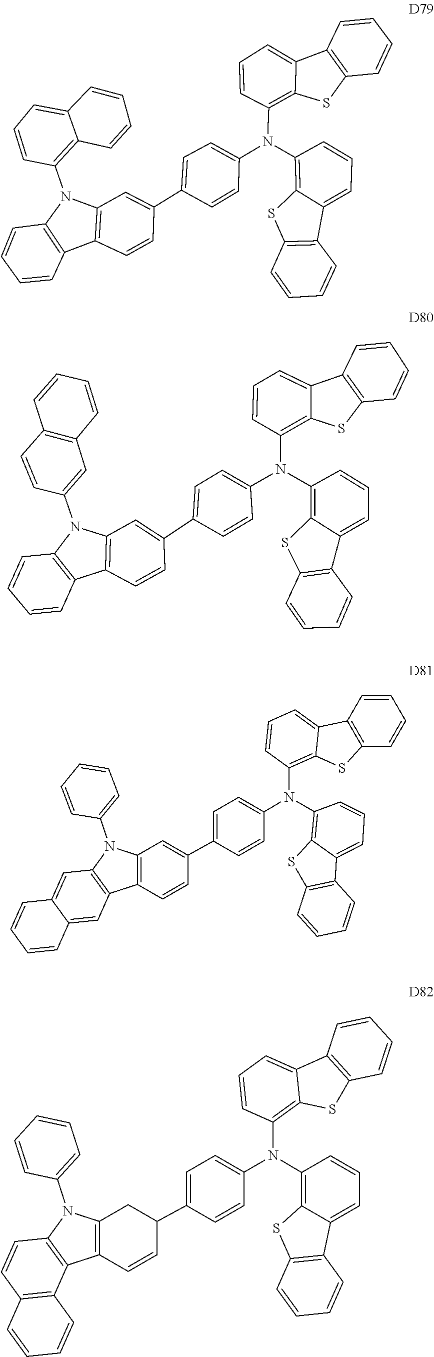

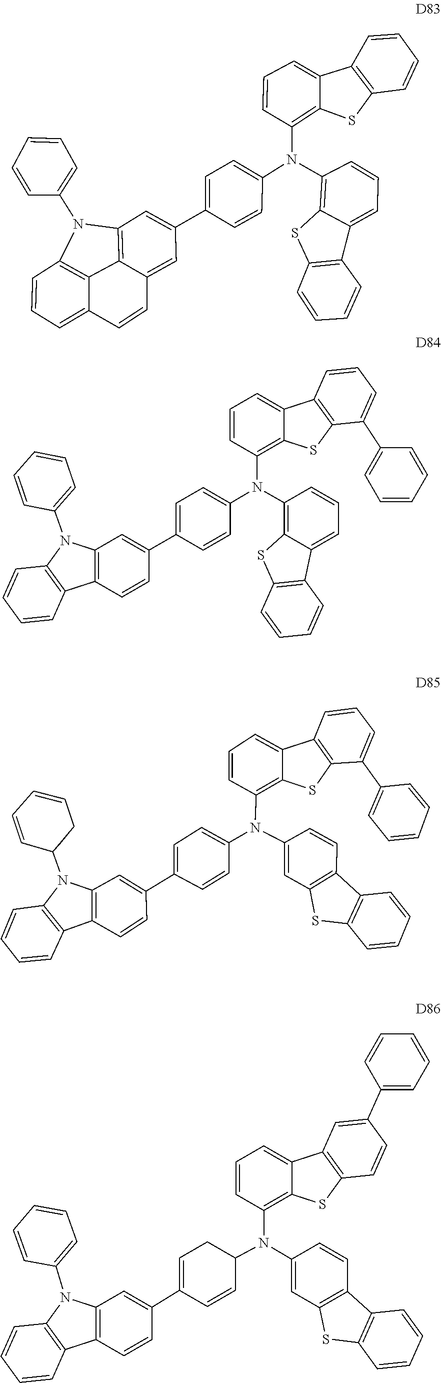 [SMILES: c1ccc(-n2c3cccc4ccc5cc(-c6ccc(N(c7cccc8c7sc7ccccc78)c7cccc8c7sc7ccccc78)cc6)cc2c5c43)cc1 ; c1ccc(-c2cccc3c2sc2c(N(c4ccc(-c5ccc6c7ccccc7n(-c7ccccc7)c6c5)cc4)c4cccc5c4sc4ccccc45)cccc23)cc1 ; C1=CC2CC2(n2c3ccccc3c3ccc(-c4ccc(N(c5ccc6c(c5)sc5ccccc56)c5cccc6c5sc5c(-c7ccccc7)cccc56)cc4)cc32)C=C1 ; C1=CC(N(c2ccc3c(c2)sc2ccccc23)c2cccc3c2sc2ccc(-c4ccccc4)cc23)CC=C1c1ccc2c3ccccc3n(-c3ccccc3)c2c1]

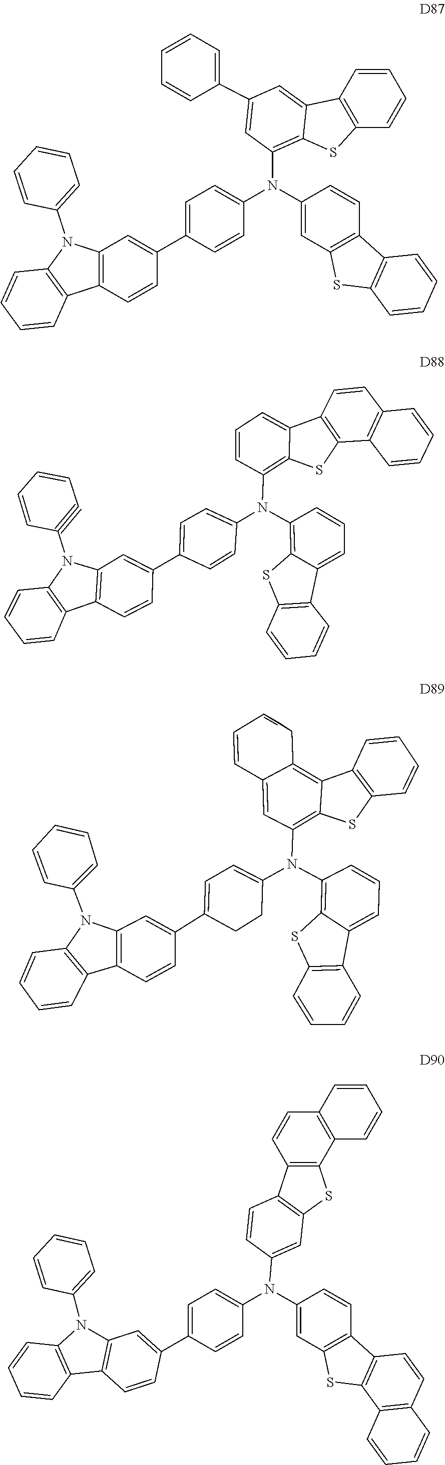

For the amine compound of an embodiment, the carbazole moiety having a high hole transport property is bonded to the nitrogen atom (N) moiety of the amine at the second position of the carbazole, and thus a highest occupied molecular orbital (HOMO) is greatly extended. Accordingly, the amine compound of an embodiment may have a stabilized radical or radical cation species. In some embodiments, in the amine compound, two dibenzoheterole moieties are bonded to the nitrogen atom (N) moiety, and thus the hole transport property may be increased, and the electron resistance and exciton resistance may be further improved. Thus, when the amine compound of an embodiment is utilized as a luminescent material, the high efficiency and long service life of the light emitting element may be realized.

The hole transport region HTR in the light emitting element ED of an embodiment may further include a compound represented by Formula H-1:

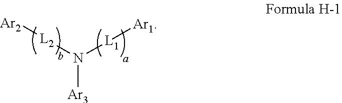

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. a and b may each independently be an integer of 0 to 10. In some embodiments, when a or b is an integer of 2 or greater, a plurality of L₁'s and L₂'s may each independently be a substituted or unsubstituted arylene group having 6 to 30 ring-forming carbon atoms, or a substituted or unsubstituted heteroarylene group having 2 to 30 ring-forming carbon atoms.

In Formula H-1, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. In some embodiments, in Formula H-1, Ar₃ may be a substituted or unsubstituted aryl group having 6 to 30 ring-forming carbon atoms.

The compound represented by Formula H-1 may be a monoamine compound. In some embodiments, the compound represented by Formula H-1 may be a diamine compound in which at least one selected from among Ar₁ to Ar₃ includes the amine group as a substituent. In some embodiments, the compound represented by Formula H-1 may be a carbazole-based compound including a substituted or unsubstituted carbazole group in at least one of Ar₁ or Ar₂, or a fluorene-based compound including a substituted or unsubstituted fluorene group in at least one of Ar₁ or Ar₂.

The compound represented by Formula H-1 may be represented by any one selected from among the compounds of Compound Group H. However, the compounds listed in Compound Group H are examples, and the compounds represented by Formula H-1 are not limited to those represented by Compound Group H:

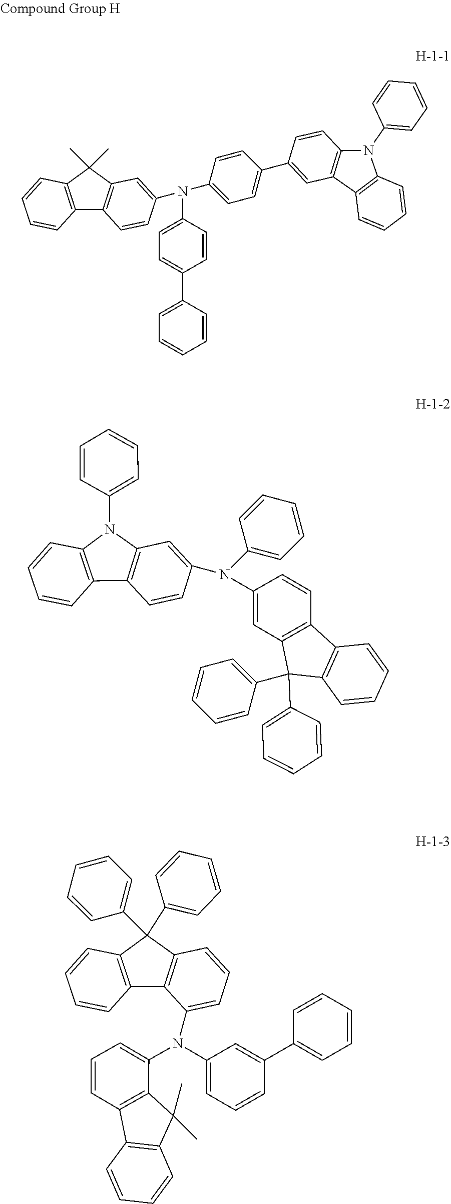 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21]

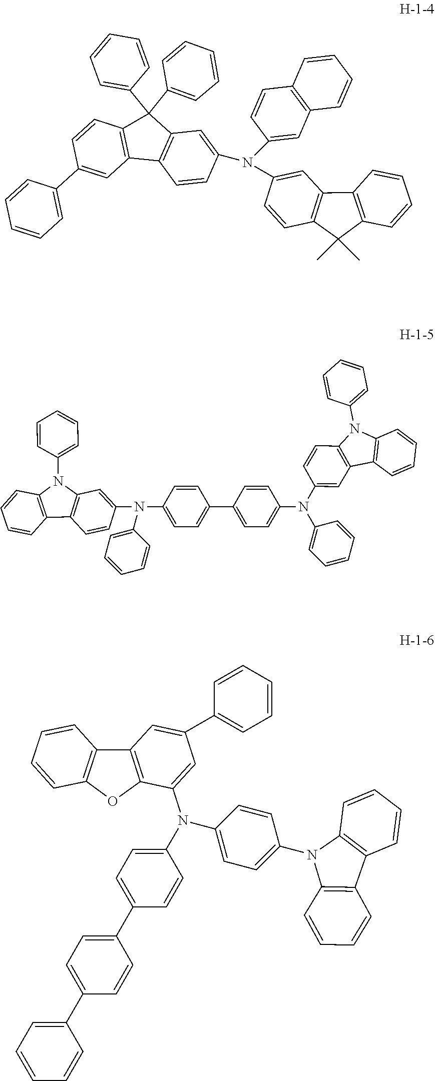 [SMILES: CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21 ; c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1]

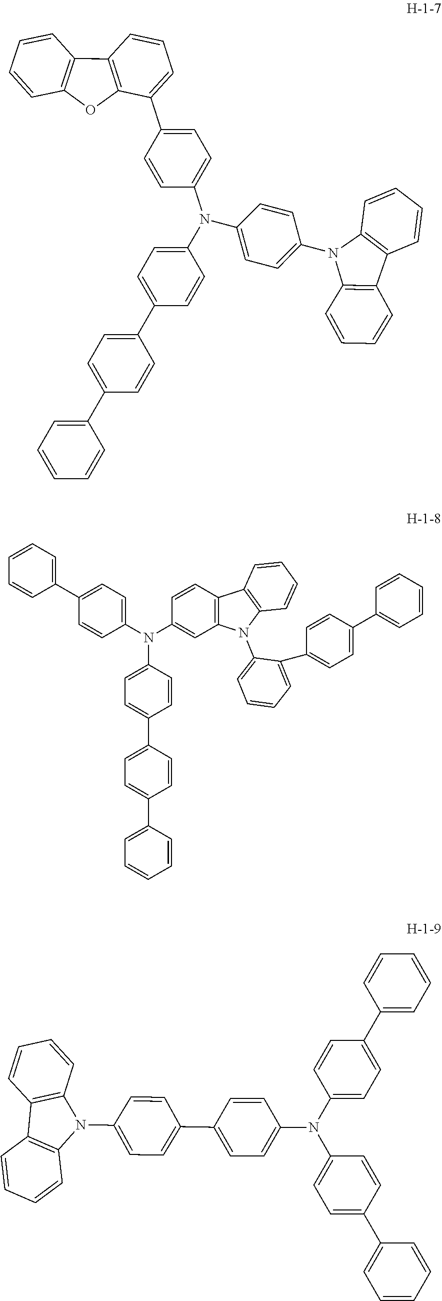 [SMILES: c1ccc(-c2ccc(-c3ccc(N(c4ccc(-c5cccc6c5oc5ccccc56)cc4)c4ccc(-n5c6ccccc6c6ccccc65)cc4)cc3)cc2)cc1 ; c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

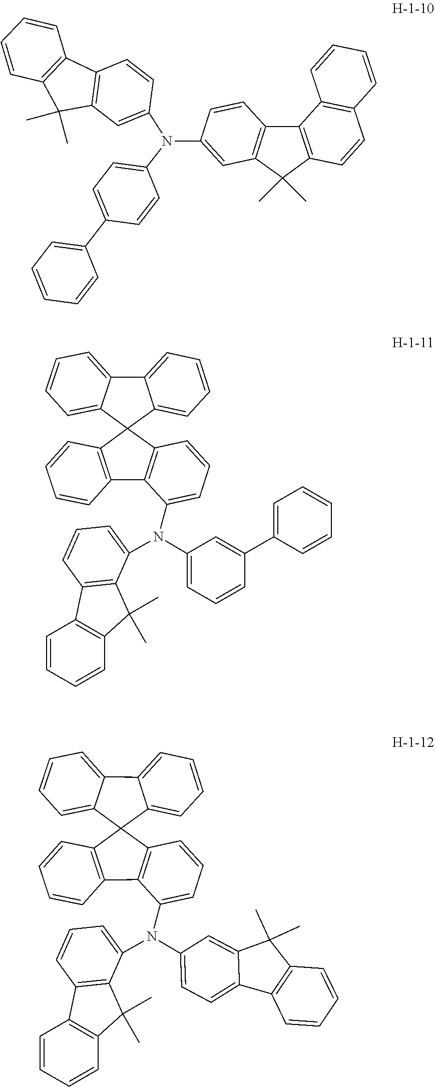 [SMILES: CC1(C)c2ccccc2-c2ccc(N(c3ccc(-c4ccccc4)cc3)c3ccc4c(c3)C(C)(C)c3ccc5ccccc5c3-4)cc21 ; CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21]

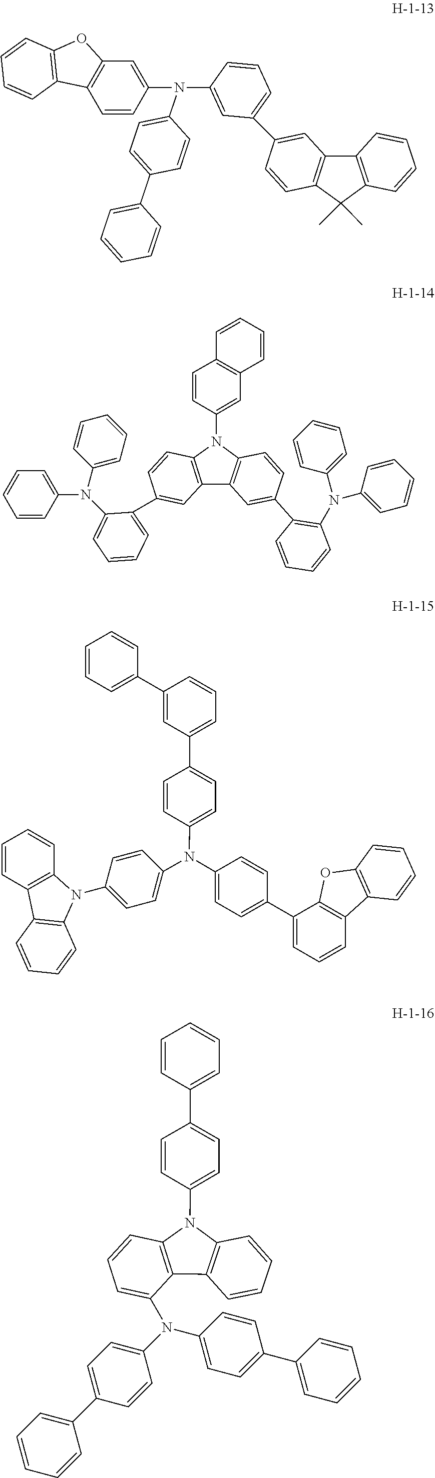 [SMILES: CC1(C)c2ccccc2-c2cc(-c3cccc(N(c4ccc(-c5ccccc5)cc4)c4ccc5c(c4)oc4ccccc45)c3)ccc21 ; c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

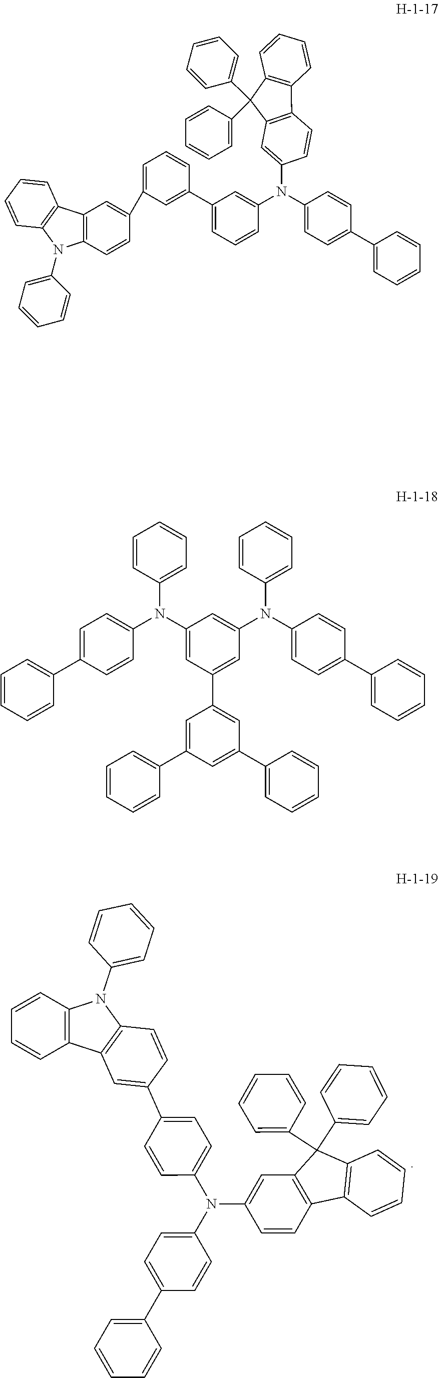 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may include a phthalocyanine compound such as copper phthalocyanine; N¹,N^{1′}-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino]triphenylamine (m-MTDATA), 4,4′,4″-tris(N,N-diphenylamino)triphenylamine (TDATA), 4,4′,4″-tris[N-(1-naphthyl)-N-phenylamino]-triphenylamine (1-TNATA), 4,4′,4″-tris[N-(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalene-I-yl)-N,N′-diphenyl-benzidine (NPB or NPD), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HATCN), etc.

The hole transport region HTR may further include a carbazole-based derivative such as N-phenyl carbazole or polyvinyl carbazole, a fluorene-based derivative, a triphenyl amine-based derivative such as 4,4′,4″-tris(N-carbazolyl)triphenylamine (TCTA), or N,N′-bis(3-methylphenyl)-N,N′-diphenyl-[1,1′-biphenyl]-4,4′-diamine (TPD), N,N′-di(naphthalene-1-yl)-N,N′-diphenyl-benzidine (NPB), 4,4′-cyclohexylidene bis[N,N-bis(4-methylphenyl)benzenamine] (TAPC), 4,4′-bis[N,N′-(3-tolyl)amino]-3,3′-dimethylbiphenyl (HMTPD), 1,3-bis(N-carbazolyl)benzene (mCP), etc.

In some embodiments, the hole transport region HTR may further include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the above-described compounds of the hole transport region HTR in at least one of a hole injection layer HIL, a hole transport layer HTL, or an electron blocking layer EBL.

The thickness of the hole transport region HTR may be from about 100 Å to about 10,000 Å, for example, from about 100 Å to about 5,000 Å. When the hole transport region HTR includes the hole injection layer HIL, the hole injection layer HIL may have, for example, a thickness of about 30 Å to about 1,000 Å. When the hole transport region HTR includes the hole transport layer HTL, the hole transport layer HTL may have a thickness of about 250 Å to about 1,000 Å. For example, when the hole transport region HTR includes the electron blocking layer EBL, the electron blocking layer EBL may have a thickness of about 10 Å to about 1,000 Å. When the thicknesses of the hole transport region HTR, the hole injection layer HIL, the hole transport layer HTL and the electron blocking layer EBL satisfy the above-described ranges, satisfactory (suitable) hole transport properties may be achieved without a substantial increase in driving voltage.

The hole transport region HTR may further include a charge generating material to increase conductivity in addition to the above-described materials. The charge generating material may be dispersed substantially uniformly or non-uniformly in the hole transport region HTR. The charge generating material may be, for example, a p-dopant. The p-dopant may include at least one of a halogenated metal compound, a quinone derivative, a metal oxide, or a cyano group-containing compound, but the embodiment of the present disclosure is not limited thereto. For example, the p-dopant may include a metal halide compound such as CuI or RbI, a quinone derivative such as tetracyanoquinodimethane (TCNQ) or 2,3,5,6-tetrafluoro-7,7, 8,8-tetracyanoquinodimethane (F4-TCNQ), a metal oxide such as tungsten oxide or molybdenum oxide, a cyano group-containing compound such as dipyrazino[2,3-f: 2′,3′-h] quinoxaline-2,3,6,7,10,11-hexacarbonitrile (HATCN) or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., but the embodiment of the present disclosure is not limited thereto.

As described above, the hole transport region HTR may further include at least one of the buffer layer or the electron blocking layer EBL in addition to the hole injection layer HIL and the hole transport layer HTL. The buffer layer may compensate for a resonance distance according to the wavelength of light emitted from the emission layer EML and may thus increase light emission efficiency. A material that may be contained in the hole transport region HTR may be utilized as a material to be contained in the buffer layer. The electron blocking layer EBL is a layer that serves to prevent or reduce the electron injection from the electron transport region ETR to the hole transport region HTR.

The emission layer EML is provided on the hole transport region HTR. The emission layer EML may have a thickness of, for example, about 100 Å to about 1,000 Å or about 100 Å to about 300 Å. The emission layer EML may have a single layer formed of a single material, a single layer formed of a plurality of different materials, or a multilayer structure having a plurality of layers formed of a plurality of different materials.

The emission layer EML in the light emitting element ED of an embodiment may emit blue light. The light emitting element ED of an embodiment may include the above-described amine compound of an embodiment in the hole transport region HTR, thereby exhibiting high efficiency and long service life characteristics in the blue light emitting region. However, the embodiment of the present disclosure is not limited thereto.

In the light emitting element ED of an embodiment, the emission layer EML may include an anthracene derivative, a pyrene derivative, a fluoranthene derivative, a chrysene derivative, a dehydrobenzanthracene derivative, or a triphenylene derivative. For example, the emission layer EML may include the anthracene derivative or the pyrene derivative.

In each of the light emitting elements ED of embodiments illustrated in FIGS. 3 to 6, the emission layer EML may include a host and a dopant, and the emission layer EML may include a compound represented by Formula E-1. The compound represented by Formula E-1 may be utilized as a fluorescent host material.

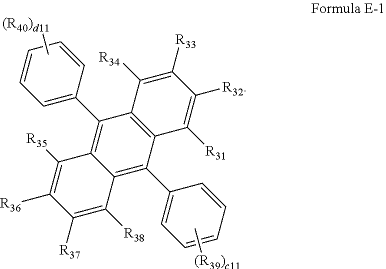

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or may be bonded to an adjacent group to form a ring. In some embodiments, R₃₁ to R₄₀ may be bonded to an adjacent group to form a saturated hydrocarbon ring or an unsaturated hydrocarbon ring, a saturated heterocycle, or an unsaturated heterocycle.

In Formula E-1, c11 and d11 may each independently be an integer from 0 to 5.

Formula E-1 may be represented by any one selected from among Compound E1 to Compound E19:

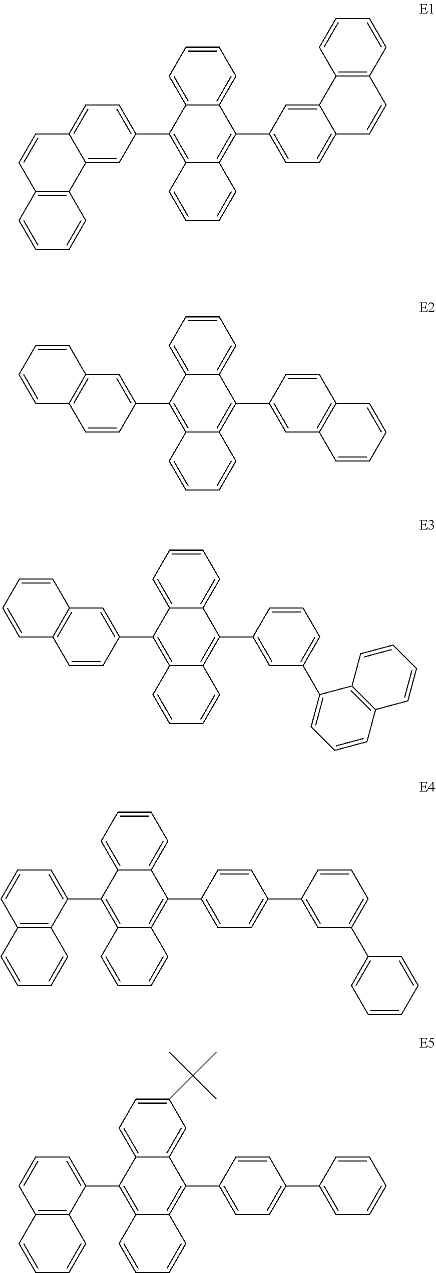

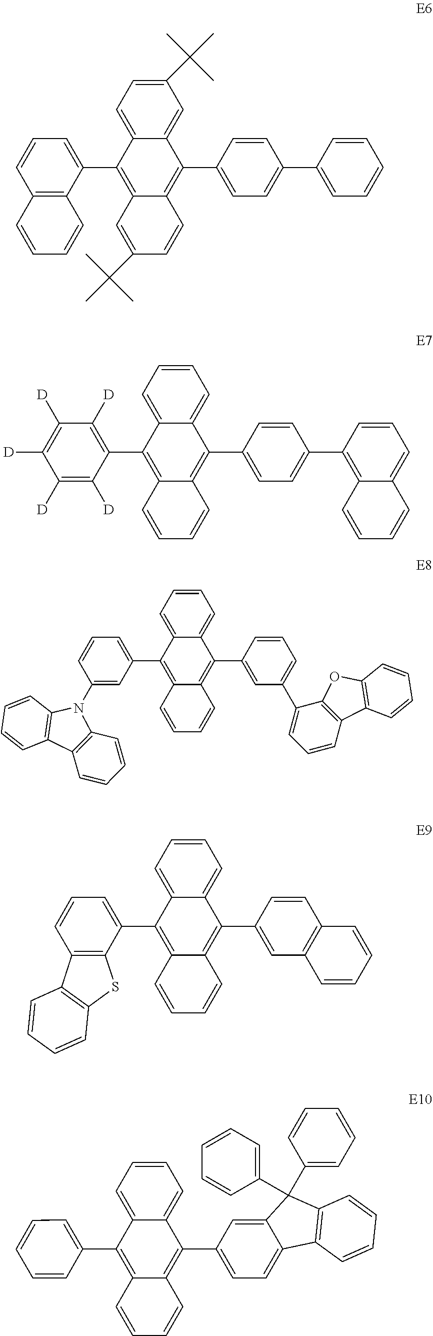

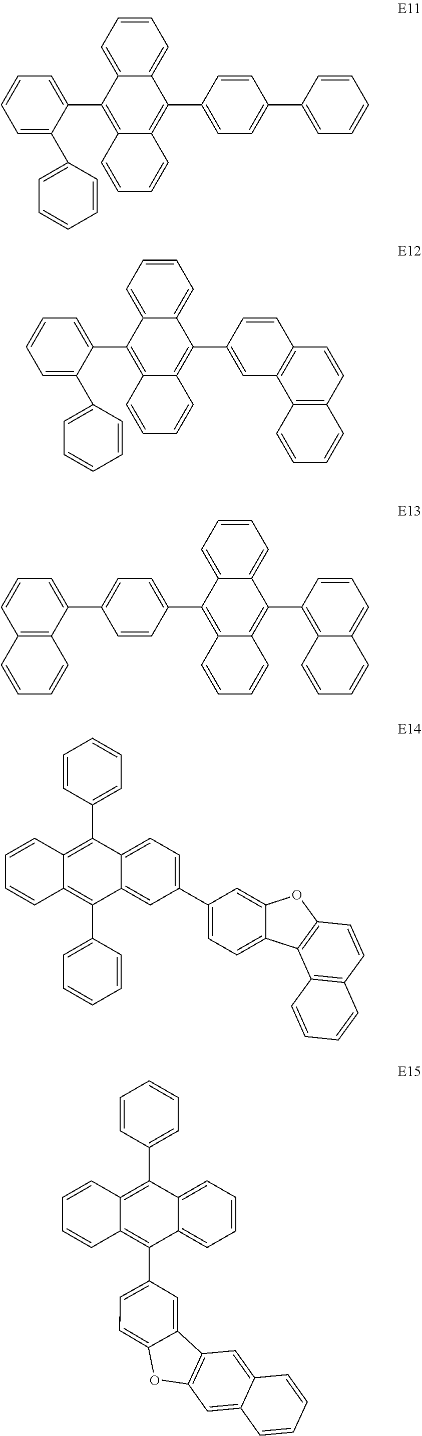

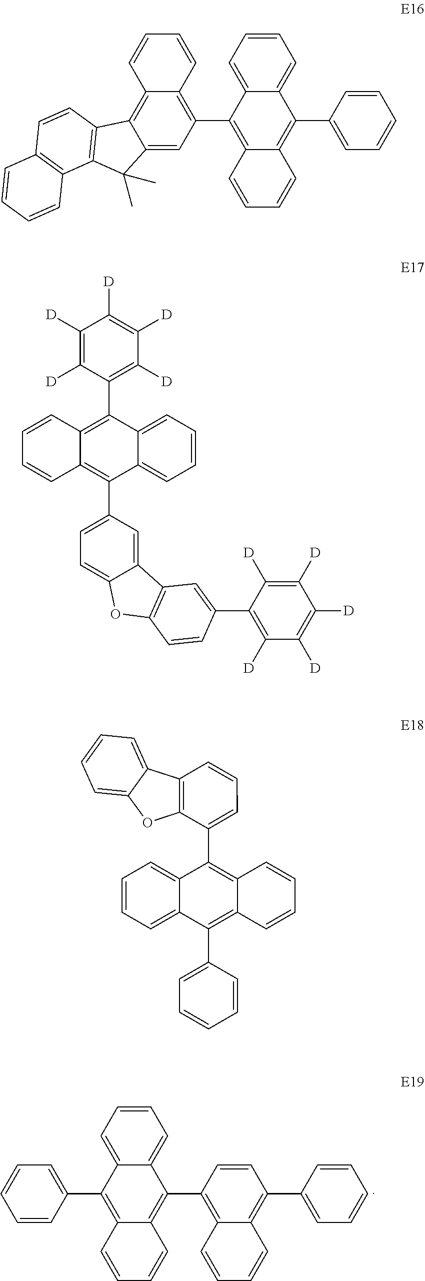

In an embodiment, the emission layer EML may include a compound represented by Formula E-2a or Formula E-2b. The compound represented by Formula E-2a or Formula E-2b may be utilized as a phosphorescent host material.

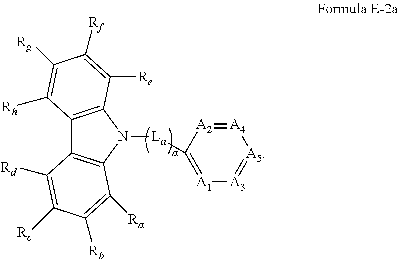

In Formula E-2a, a may be an integer from 0 to 10, Lₐ may be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a is an integer of 2 or more, a plurality of Lₐs may each independently be a substituted or unsubstituted arylene group having 6 to 30 ring-forming carbon atoms, or a substituted or unsubstituted heteroarylene group having 2 to 30 ring-forming carbon atoms.

In some embodiments, in Formula E-2a, A₁ to A₅ may each independently be N or CRᵢ. Rₐ to R₁ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may be bonded to an adjacent group to form a ring. Rₐ to Rᵢ may be bonded to an adjacent group to form a hydrocarbon ring or a heterocycle containing N, O, S, etc. as a ring-forming atom.

In some embodiments, in Formula E-2a, two or three selected from among A₁ to A₅ may be N, and the rest may be CRᵢ.

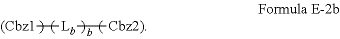

In Formula E-2b, Cbz1 and Cbz2 may each independently be an unsubstituted carbazole group, or a carbazole group substituted with an aryl group having 6 to 30 ring-forming carbon atoms. L_{b} may be a direct linkage, a substituted or unsubstituted arylene group having 6 to 30 ring-forming carbon atoms, or a substituted or unsubstituted heteroarylene group having 2 to 30 ring-forming carbon atoms. In some embodiments, b is an integer from 0 to 10, and when b is an integer of 2 or more, a plurality of L_{b}s may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by any one selected from among the compounds of Compound Group E-2. However, the compounds listed in Compound Group E-2 are merely examples, and the compound represented by Formula E-2a or Formula E-2b is not limited to those represented in Compound Group E-2.

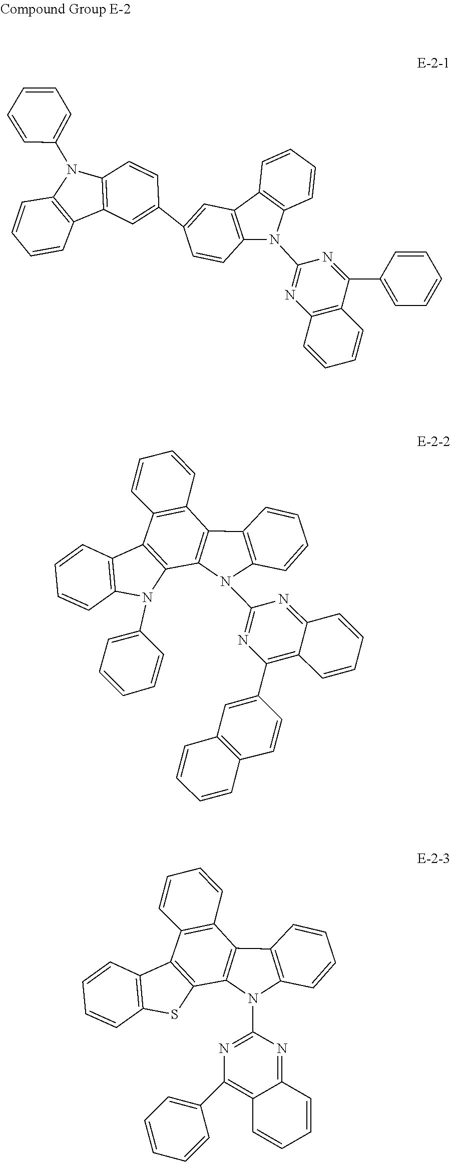

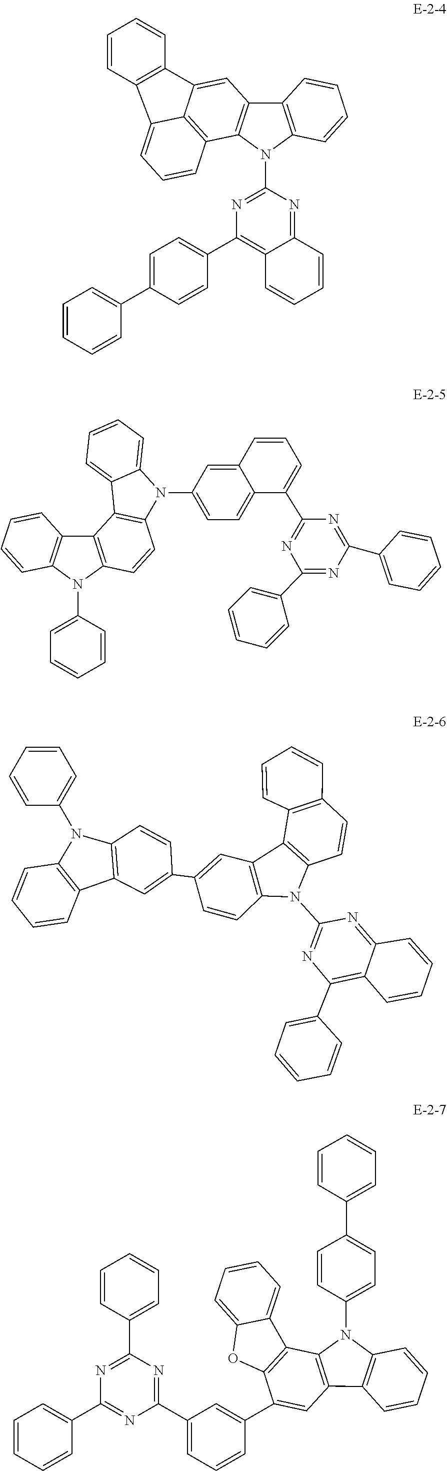 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1]

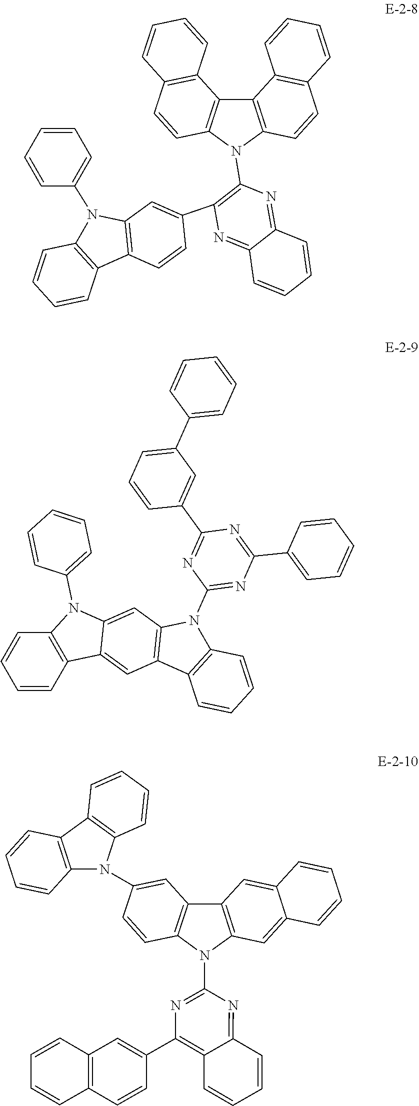 [SMILES: c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1]

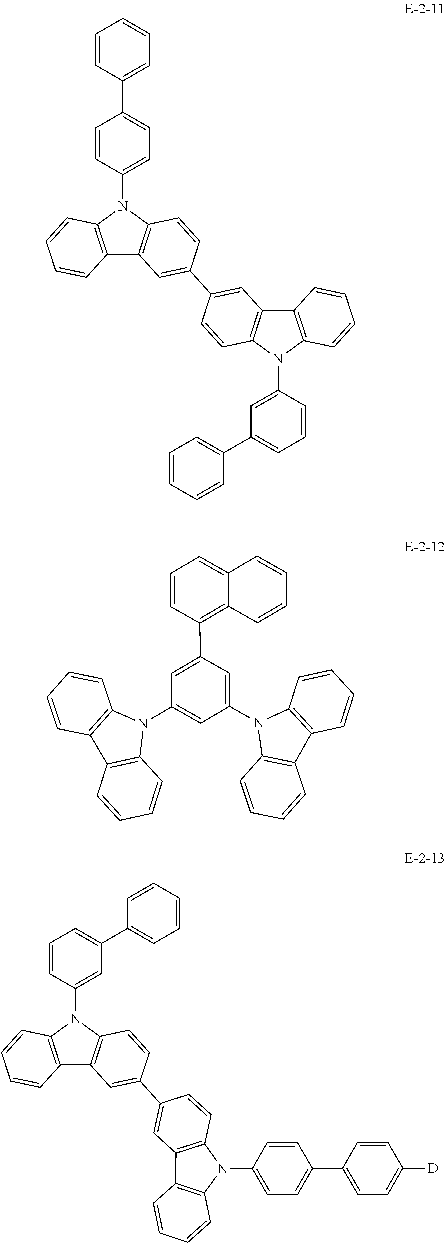 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1]

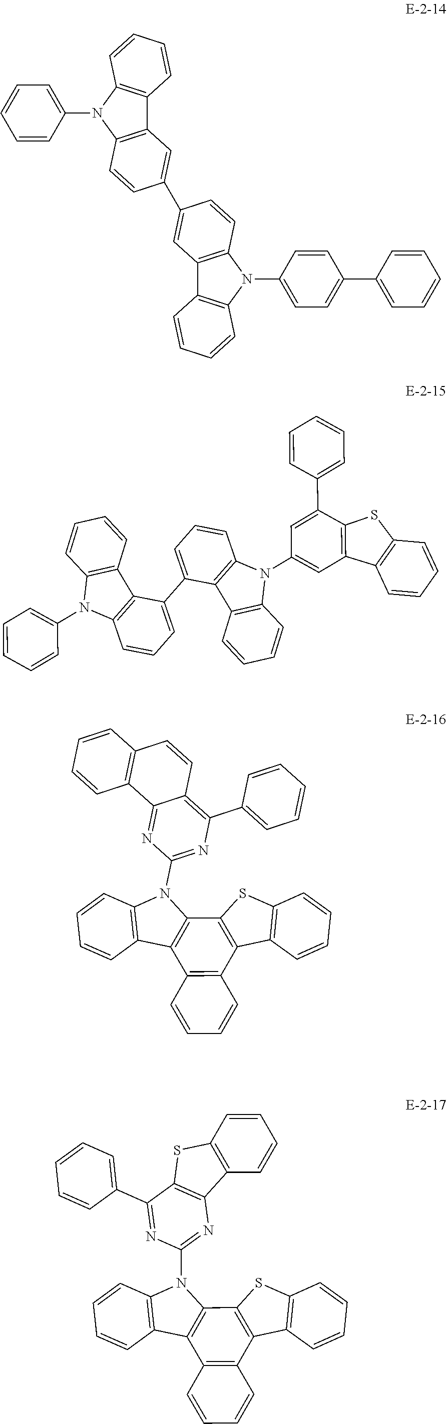 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1]

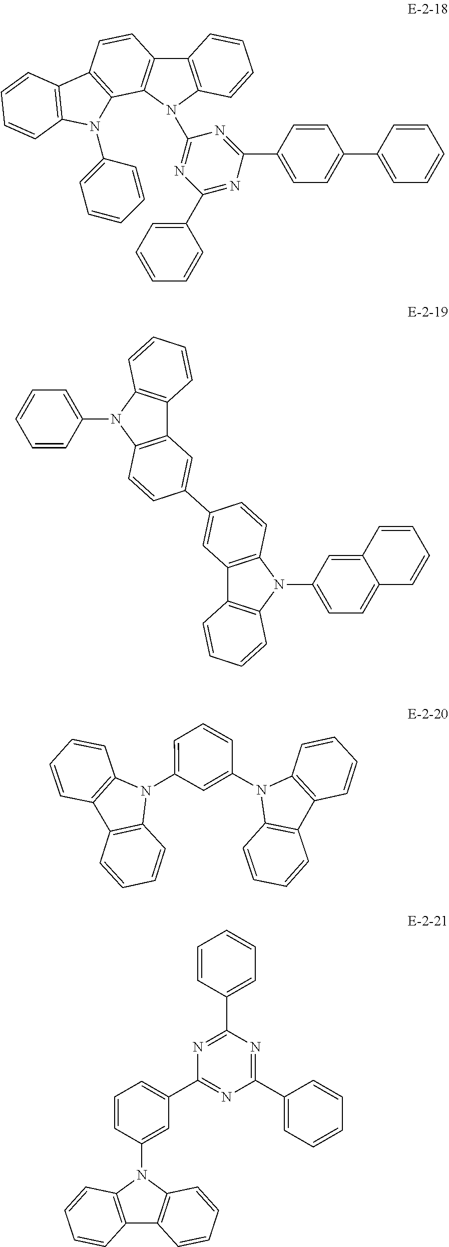 [SMILES: c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1]

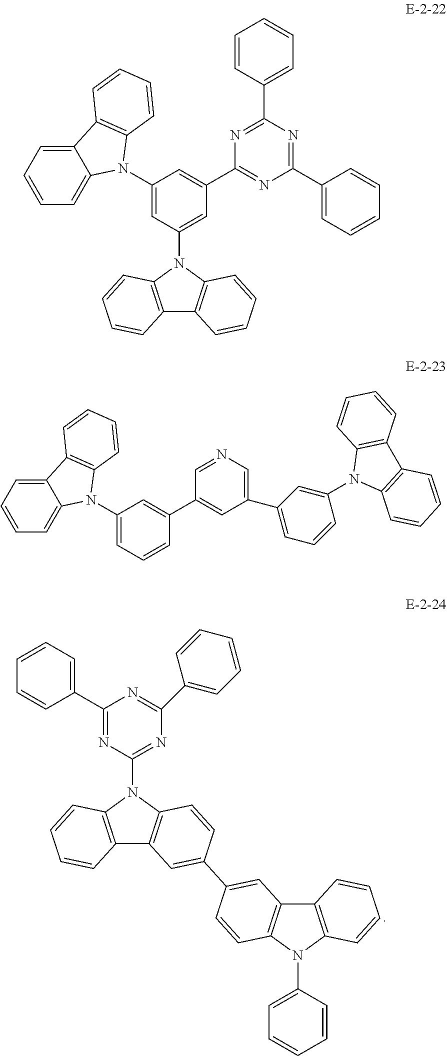 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a general material suitable in the art as a host material. For example, the emission layer EML may include, as a host material, at least one of bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, the embodiment of the present disclosure is not limited thereto, for example, tris(8-hydroxyquinolino)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be utilized as a host material.

The emission layer EML may include a compound represented by Formula M-a or Formula M-b. The compound represented by Formula M-a or Formula M-b may be utilized as a phosphorescent dopant material. In some embodiments, the compound represented by Formula M-a or Formula M-b in an embodiment may be utilized as an auxiliary dopant material.

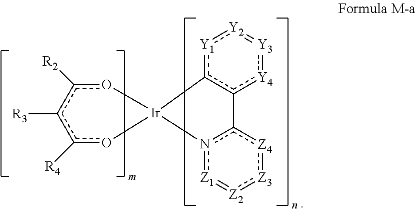

In Formula M-a, Y₁ to Y₄ and Z₁ to Z₄ may each independently be CR₁ or N, R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may be bonded to an adjacent group to form a ring. In Formula M-a, m may be 0 or 1, and n may be 2 or 3. In Formula M-a, when m is 0, n is 3, and when m is 1, n is 2.

The compound represented by Formula M-a may be represented by any one selected from among Compound M-a1 to Compound M-a25. However, Compounds M-a1 to M-a25 are examples, and the compound represented by Formula M-a is not limited to those represented by Compounds M-a1 to M-a25.

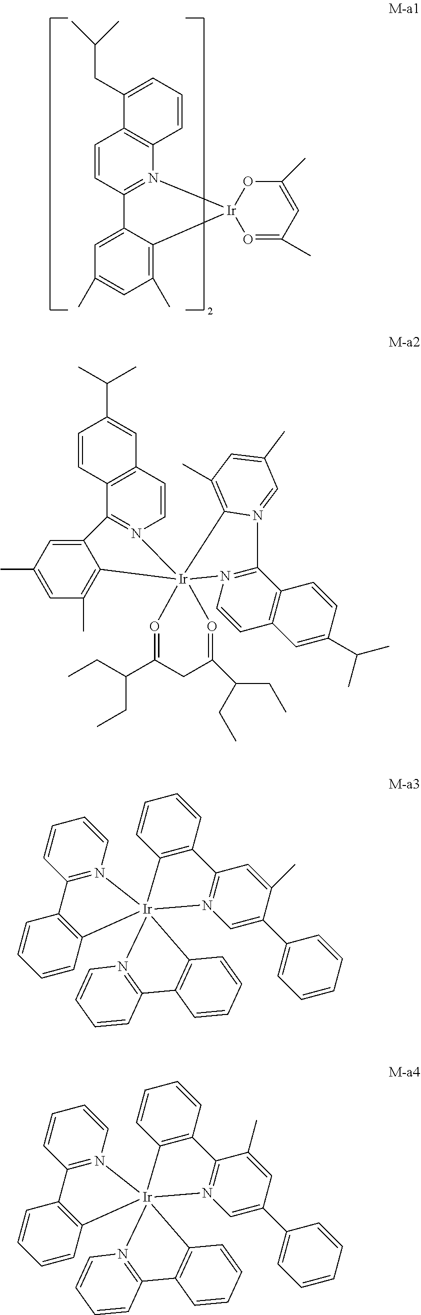

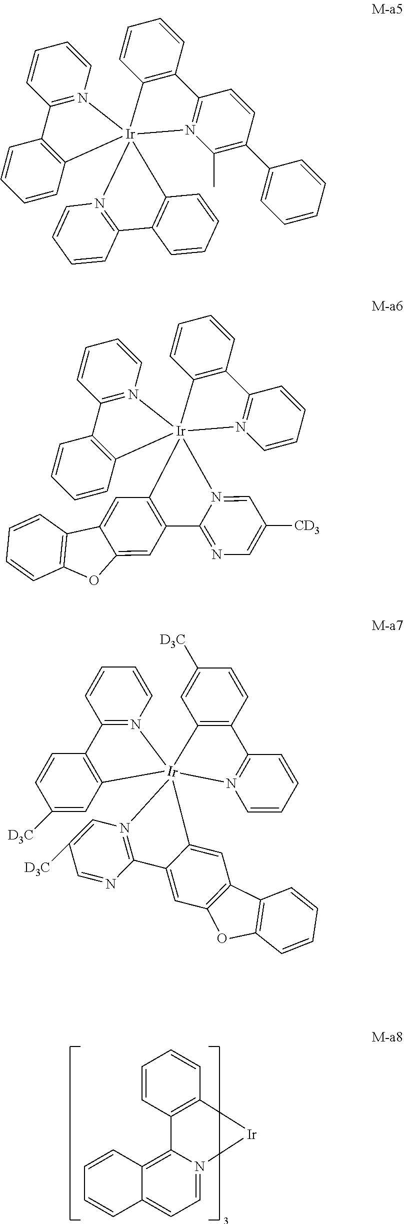

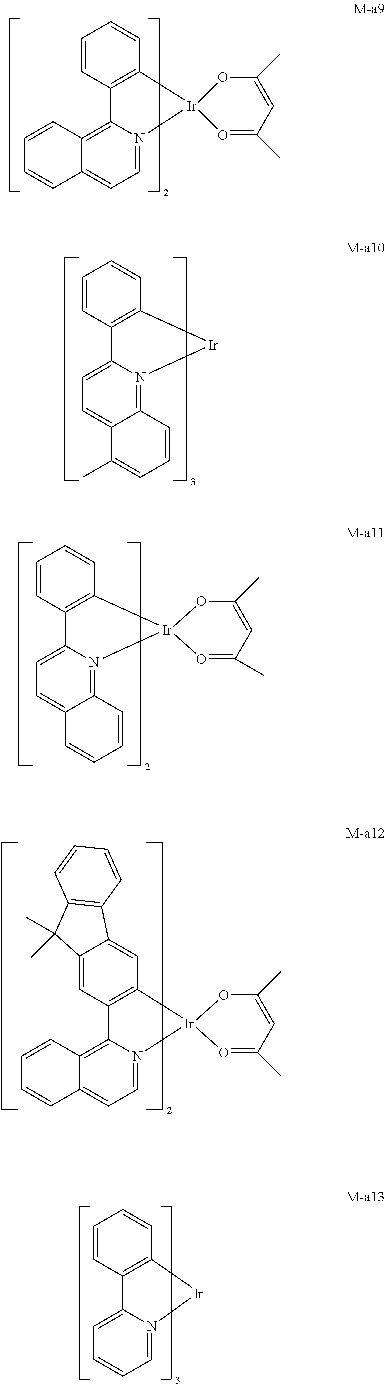

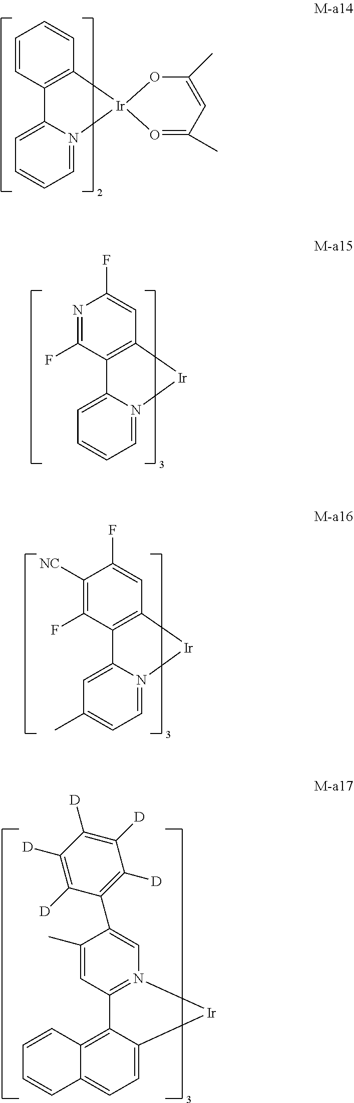

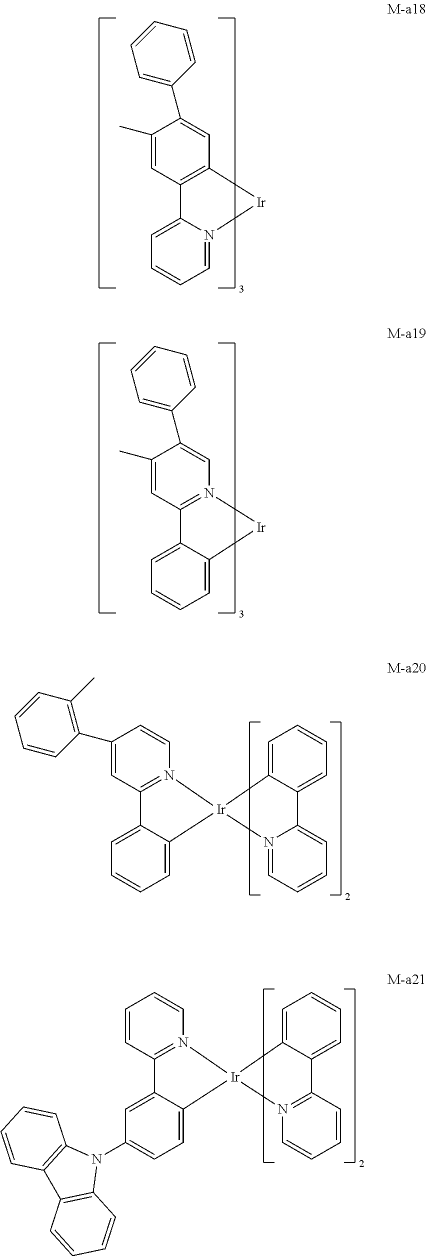

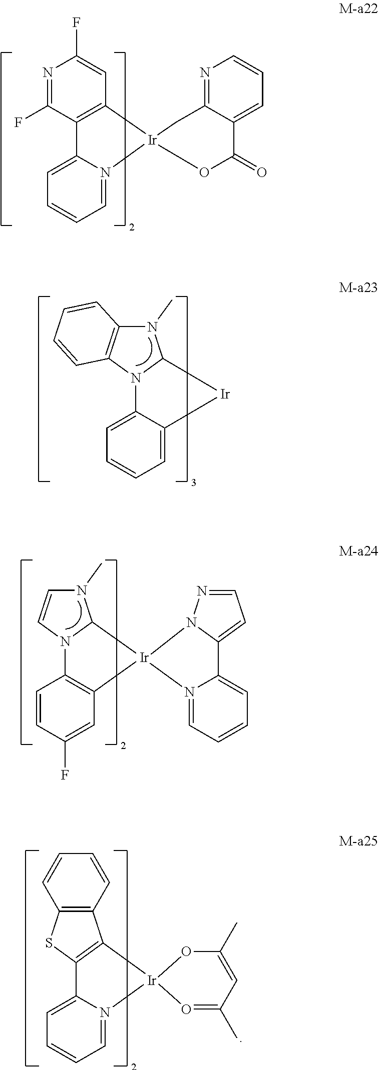

Compound M-a1 and Compound M-a2 may be utilized as a red dopant material, and Compound M-a3 to Compound M-a7 may be utilized as a green dopant material.

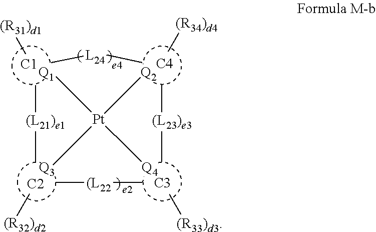

In Formula M-b, Q₁ to Q₄ may each independently be C or N, and C1 to C4 may each independently be a substituted or unsubstituted hydrocarbon ring having 5 to 30 ring-forming carbon atoms, or a substituted or unsubstituted heterocycle having 2 to 30 ring-forming carbon atoms. L₂₁ to L₂₄ may each independently be a direct linkage,

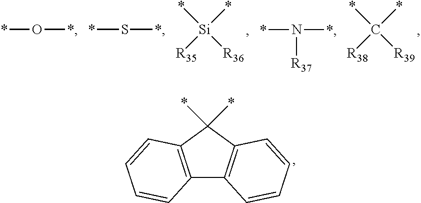

substituted or unsubstituted divalent alkyl group having 1 to 20 carbon atoms, substituted or unsubstituted arylene group having 6 to 30 ring-forming carbon atoms, or a substituted or unsubstituted heteroarylene group having 2 to 30 ring-forming carbon atoms, and e1 to e4 may each independently be 0 or 1. R₃₁ to R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or are bonded to an adjacent group to form a ring, and d1 to d4 may each independently be an integer from 0 to 4.

The compound represented by Formula M-b may be utilized as a blue phosphorescent dopant or a green phosphorescent dopant. In some embodiments, the compound represented by Formula M-b may be further included as an auxiliary dopant in the emission layer EML in an embodiment.

The compound represented by Formula M-b may be represented by any one selected from among the following compounds. However, these compounds are merely examples, and the compound represented by Formula M-b is not limited to those represented by these compounds.

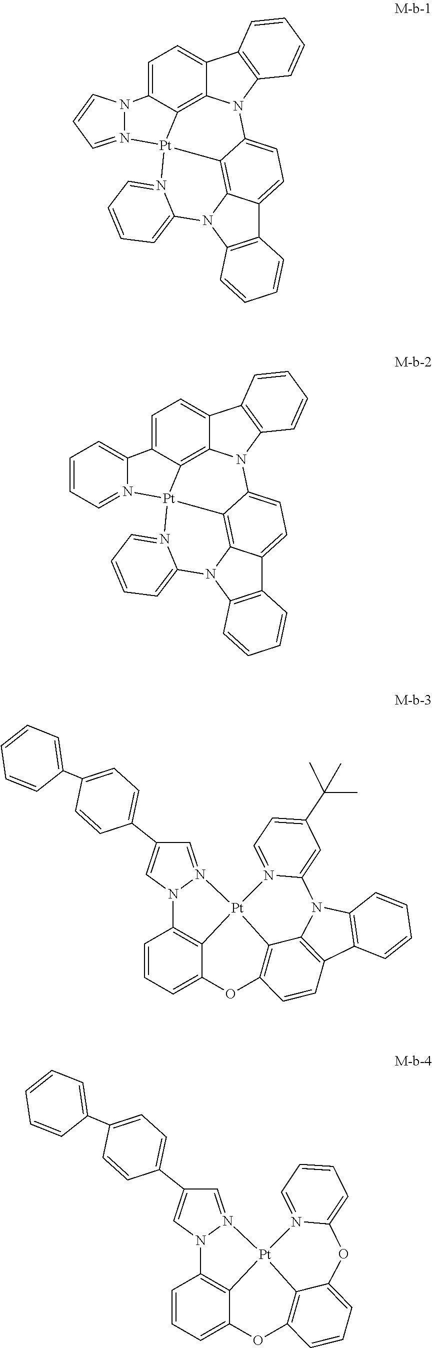

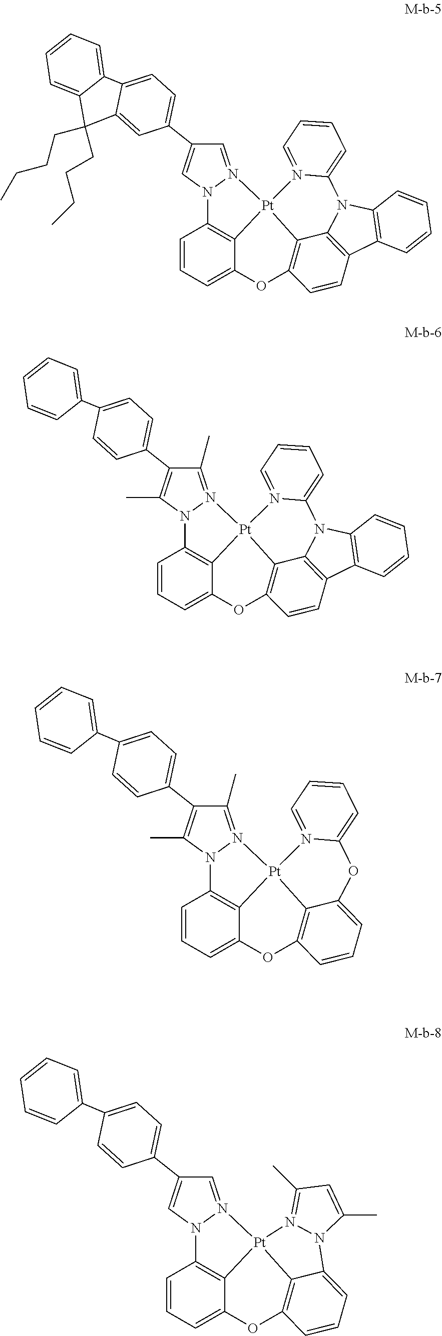

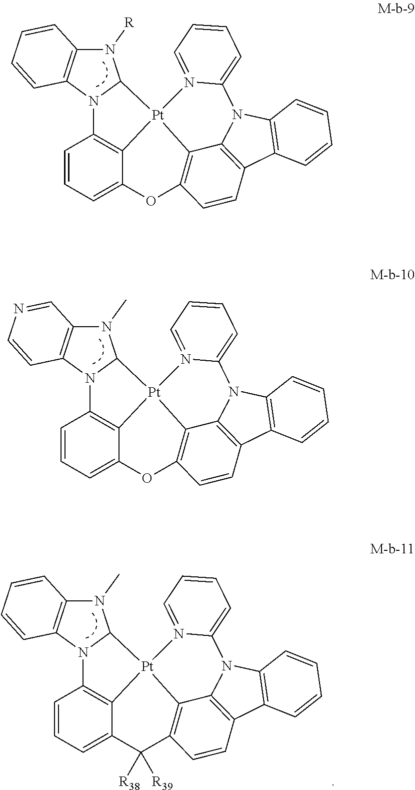

In the foregoing compounds, R, R₃₈, and R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

The emission layer EML may further include a compound represented by any one selected from among Formula F-a to Formula F-c. The compound represented by Formula F-a or Formula F-c may be utilized as a fluorescence dopant material.

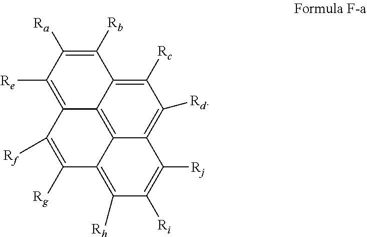

In Formula F-a, two groups selected from among Rₐ to Rⱼ may each independently be substituted with

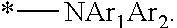

The others, which are not substituted with

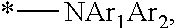

selected from among Rₐ to Rⱼ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In

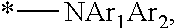

Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, at least one of Ar₁ or Ar₂ may be a heteroaryl group containing O or S as a ring-forming atom.

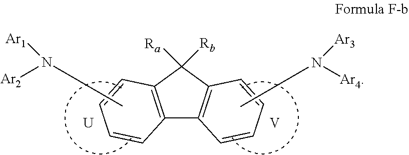

In Formula F-b, Rₐ and R_{b} may each independently be a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may be bonded to an adjacent group to form a ring. Ar₁ to Ar₄ may each independently be a substituted or unsubstituted aryl group having 6 to 30 ring-forming carbon atoms, or a substituted or unsubstituted heteroaryl group having 2 to 30 ring-forming carbon atoms.

In Formula F-b, U and V may each independently be a substituted or unsubstituted hydrocarbon ring having 5 to 30 ring-forming carbon atoms, or a substituted or unsubstituted heterocycle having 2 to 30 ring-forming carbon atoms.

In Formula F-b, the number of rings represented by U and V may each independently be 0 or 1. For example, in Formula F-b, it refers to that when the number of U or V is 1, one ring constitutes a fused ring at a portion indicated by U or V, and when the number of U or V is 0, a ring indicated by U or V does not exist. For example, when the number of U is 0 and the number of V is 1, or when the number of U is 1 and the number of V is 0, the fused ring having a fluorene core in Formula F-b may be a cyclic compound having four rings. In some embodiments, when each number of U and V is 0, the fused ring in Formula F-b may be a cyclic compound having three rings. In some embodiments, when each number of U and V is 1, the fused ring having a fluorene core in Formula F-b may be a cyclic compound having five rings.

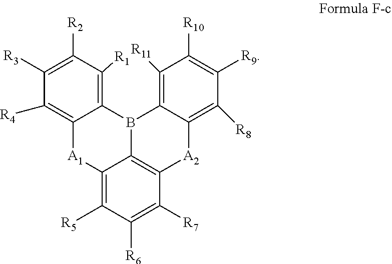

In Formula F-c, A₁ and A₂ may each independently be O, S, Se, or NRₘ, and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or are bonded to an adjacent group to form a ring.

In Formula F-c, A₁ and A₂ may each independently be bonded to substituents of an adjacent ring to form a condensed ring. For example, when A₁ and A₂ may each independently be NRₘ, A₁ may be bonded to R₄ or R₅ to form a ring. In some embodiments, A₂ may be bonded to R₇ or R₈ to form a ring.

In an embodiment, the emission layer EML may include, as a suitable dopant material, styryl derivatives (e.g., 1,4-bis[2-(3-N-ethylcarbazolyl)vinyl]benzene (BCzVB), 4-(di-p-tolylamino)-4′-[(di-p-tolylamino)styryl]stilbene (DPAVB), and N-(4-((E)-2-(6-((E)-4-(diphenylamino)styryl)naphthalen-2-yl)vinyl)phenyl)-N-phenylbenzenamine (N-BDAVBi), 4,4′-bis[2-(4-(N,N-diphenylamino)phenyl)vinyl]biphenyl (DPAVBi), perylene and the derivatives thereof (e.g., 2,5,8,11-tetra-t-butylperylene (TBP)), pyrene and the derivatives thereof (e.g., 1,1-dipyrene, 1,4-dipyrenylbenzene, 1,4-bis(N,N-diphenylamino)pyrene), etc.

In an embodiment, when a plurality of emission layers EML are included, at least one emission layer EML may include a suitable phosphorescence dopant material. For example, a metal complex containing iridium (Ir), platinum (Pt), osmium (Os), aurum (Au), titanium (Ti), zirconium (Zr), hafnium (Hf), europium (Eu), terbium (Tb), or thulium (Tm) may be utilized as a phosphorescent dopant. For example, iridium(III) bis(4,6-difluorophenylpyridinato-N,C2′)picolinate (FIrpic), bis(2,4-difluorophenylpyridinato)-tetrakis(1-pyrazolyl)borate iridium(III) (FIre), or platinum octaethyl porphyrin (PtOEP) may be utilized as a phosphorescent dopant. However, the embodiment of the present disclosure is not limited thereto.

In some embodiments, the emission layer EML in an embodiment may include a hole transport host and an electron transport host. Also, the emission layer EML may include an auxiliary dopant and a light emitting dopant. In some embodiments, a phosphorescent dopant material or a thermally delayed fluorescent dopant material may be included as the auxiliary dopant. For example, the emission layer EML in an embodiment may include the hole transport host, the electron transport host, the auxiliary dopant, and the light emitting dopant.

In some embodiments, in the emission layer EML, an exciplex may be formed by the hole transport host and the electron transport host. In this embodiment, a triplet energy of the exciplex formed by the hole transport host and the electron transport host may correspond to T1 that is a gap between a lowest unoccupied molecular orbital (LUMO) energy level of the electron transport host and a HOMO energy level of the hole transport host.

In an embodiment, the triplet energy (T1) of the exciplex formed by the hole transport host and the electron transport host may be about 2.4 eV to about 3.0 eV. In some embodiments, the triplet energy of the exciplex may be a value smaller than an energy gap of each host material. Therefore, the exciplex may have a triplet energy of about 3.0 eV or less that is an energy gap between the hole transport host and the electron transport host.

In some embodiments, at least one emission layer EML may include a quantum dot material. The core of the quantum dot may be selected from a Group II-VI compound, a Group III-VI compound, a Group I-III-VI compound, a Group III-V compound, a Group III-II-V compound, a Group IV-VI compound, a Group IV element, a Group IV compound, and one or more combinations thereof.

The Group II-VI compound may be selected from the group including (e.g., consisting of) a binary compound selected from the group including (e.g., consisting of) CdSe, CdTe, CdS, ZnS, ZnSe, ZnTe, ZnO, HgS, HgSe, HgTe, MgSe, MgS, and a mixture thereof, a ternary compound selected from the group including (e.g., consisting of) CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof, and a quaternary compound selected from the group including (e.g., consisting of) CdZnSeS, CdZnSeTe, CdZnSTe, CdHgSeS, CdHgSeTe, CdHgSTe, HgZnSeS, HgZnSeTe, HgZnSTe, and a mixture thereof.

The Group III-VI compound may include a binary compound such as In₂S₃ or In₂Se₃, a ternary compound such as InGaS₃ or InGaSe₃, or one or more combinations thereof.

The Group I-III-VI compound may be selected from a ternary compound selected from the group including (e.g., consisting of) AgInS, AgInS₂, CuInS, CuInS₂, AgGaS₂, CuGaS₂ CuGaO₂, AgGaO₂, AgAlO₂, and one or more compounds or mixtures thereof, and/or a quaternary compound such as AgInGaS₂ or CuInGaS₂ (the quaternary compound may be used alone or in combination with any of the foregoing compounds or mixtures; and the quaternary compound may also be combined with other quaternary compounds).

The Group III-V compound may be selected from the group including (e.g., consisting of) a binary compound selected from the group including (e.g., consisting of) GaN, GaP, GaAs, GaSb, AlN, AlP, AlAs, AlSb, InN, InP, InAs, InSb, and one or more compounds or mixtures thereof, a ternary compound selected from the group including (e.g., consisting of) GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and one or more compounds or mixtures thereof, and a quaternary compound selected from the group including (e.g., consisting of) GaAlNP, GaAlNAs, GaAlNSb, GaAlPAs, GaAlPSb, GaInNP, GaInNAs, GalnNSb, GaInPAs, GalnPSb, InAlNP, InAlNAs, InAlNSb, InAlPAs, InAlPSb, and one or more compounds or mixtures thereof. In some embodiments, the Group III-V compound may further include a Group II metal. For example, InZnP, etc. may be selected as a Group III-II-V compound.

The Group IV-VI compound may be selected from the group including (e.g., consisting of) a binary compound selected from the group including (e.g., consisting of) SnS, SnSe, SnTe, PbS, PbSe, PbTe, and one or more compounds or mixtures thereof, a ternary compound selected from the group including (e.g., consisting of) SnSeS, SnSeTe, SnSTe, PbSeS, PbSeTe, PbSTe, SnPbS, SnPbSe, SnPbTe, and one or more compounds or mixtures thereof, and a quaternary compound selected from the group including (e.g., consisting of) SnPbSSe, SnPbSeTe, SnPbSTe, and one or more compounds or mixtures thereof. The Group IV element may be selected from the group including (e.g., consisting of) Si, Ge, and one or more elements or mixtures thereof. The Group IV compound may be a binary compound selected from the group including (e.g., consisting of) SiC, SiGe, and one or more compounds or mixtures thereof.

In this embodiment, a binary compound, a ternary compound, or a quaternary compound may be present in a particle form and the particle being with a uniform concentration distribution, or may be present in the same particle with a partially different concentration distribution. In some embodiments, a core/shell structure in which one quantum dot surrounds another quantum dot may also be possible. The core/shell structure may have a concentration gradient in which the concentration of elements present in the shell decreases toward the core.

In some embodiments, the quantum dot may have the above-described core/shell structure including a core containing nanocrystals and a shell around (e.g., surrounding) the core. The shell of the quantum dot may serve as a protection layer to prevent or reduce the chemical deformation of the core so as to maintain semiconductor properties, and/or a charging layer to impart electrophoresis properties to the quantum dot. The shell may be a single layer or a multilayer. An example of the shell of the quantum dot may include a metal or non-metal oxide, a semiconductor compound, or a combination thereof.

For example, the metal or non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, CO₃O₄, and/or NiO, or a ternary compound such as MgAl₂O₄, CoFe₂O₄, NiFe₂O₄, and CoMn₂O₄, but the present present disclosure is not limited thereto.

In some embodiments, the semiconductor compound may be, for example, CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but the embodiment of the present disclosure is not limited thereto.

The quantum dot may have a full width of half maximum (FWHM) of a light emitting wavelength spectrum of about 45 nm or less, about 40 nm or less, or about 30 nm or less, and color purity or color reproducibility may be improved in the above range. In some embodiments, light emitted through such a quantum dot is emitted in all directions, and thus a wide viewing angle may be improved (increased).

In some embodiments, although the form of a quantum dot is not limited as long as it is a form commonly utilized in the art, more specifically, a quantum dot in the form of a substantially spherical, pyramidal, multi-arm, or cubic nanoparticles, nanotubes, nanowires, nanofibers, nanoparticles, etc. may be utilized.

The quantum dot may control the color of emitted light according to the particle size thereof. Accordingly, the quantum dot may have one or more suitable light emission colors such as blue, red, and/or green.

In each of the light emitting elements ED of embodiments illustrated in FIGS. 3 to 6, the electron transport region ETR is provided on the emission layer EML. The electron transport region ETR may include at least one of the hole blocking layer HBL, the electron transport layer ETL, or the electron injection layer EIL, but the embodiment of the present disclosure is not limited thereto.

The electron transport region ETR may have a single layer formed of a single material, a single layer formed of a plurality of different materials, or a multilayer structure including a plurality of layers formed of a plurality of different materials.

For example, the electron transport region ETR may have a single layer structure of the electron injection layer EIL or the electron transport layer ETL, and may have a single layer structure formed of an electron injection material and an electron transport material. In some embodiments, the electron transport region ETR may have a single layer structure formed of a plurality of different materials, or may have a structure in which an electron transport layer ETL/electron injection layer EIL, a hole blocking layer HBL/electron transport layer ETL/electron injection layer EIL, an electron transport layer ETL/buffer layer/electron injection layer EIL are stacked in order from the emission layer EML, but the embodiment of the present disclosure is not limited thereto. The electron transport region ETR may have a thickness, for example, from about 1,000 Å to about 1,500 Å.

The electron transport region ETR may be formed by utilizing one or more suitable methods such as a vacuum deposition method, a spin coating method, a cast method, a Langmuir-Blodgett (LB) method, an inkjet printing method, a laser printing method, and a laser induced thermal imaging (LITI) method.

The electron transport region ETR may include a compound represented by Formula ET-1:

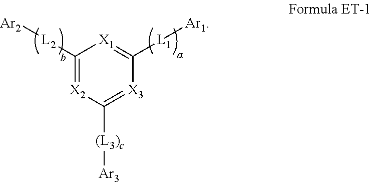

In Formula ET-1, at least one among X₁ to X₃ is N, and the rest are CRₐ. Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-1, a to c may each independently be an integer from 0 to 10. In Formula ET-1, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a to c are an integer of 2 or more, L₁ to L₃ may each independently be a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, the embodiment of the present disclosure is not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalene-2-yl)anthracene (ADN), 1,3-bis[3,5-di(pyridin-3-yl)phenyl]benzene (BmPyPhB), diphenyl(4-(triphenylsilyl)phenyl)phosphine oxide (TSPO1), or one or more compounds or mixtures thereof.

The electron transport region ETR may include at least one selected from among Compound ET1 to Compound ET36:

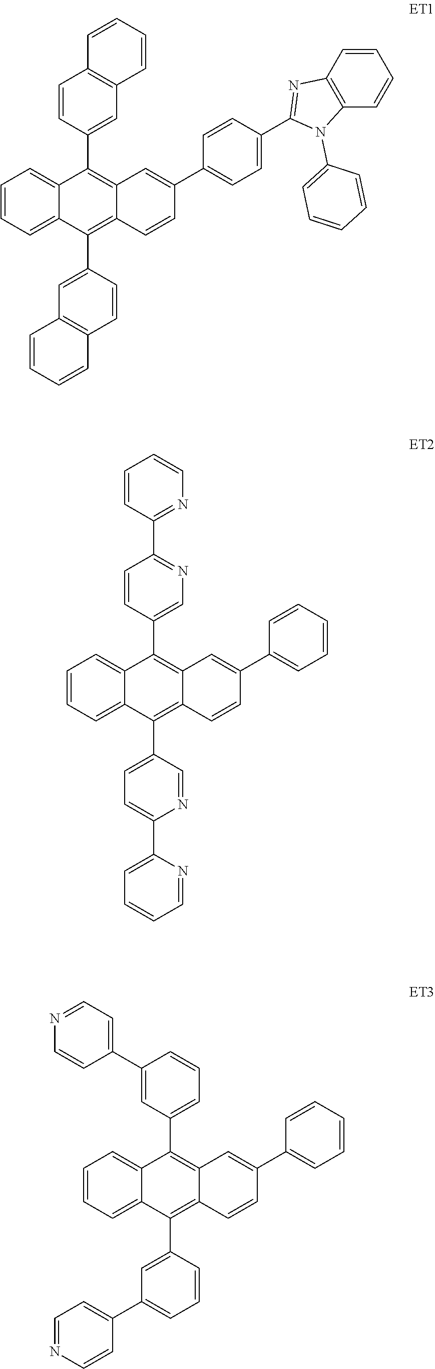 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

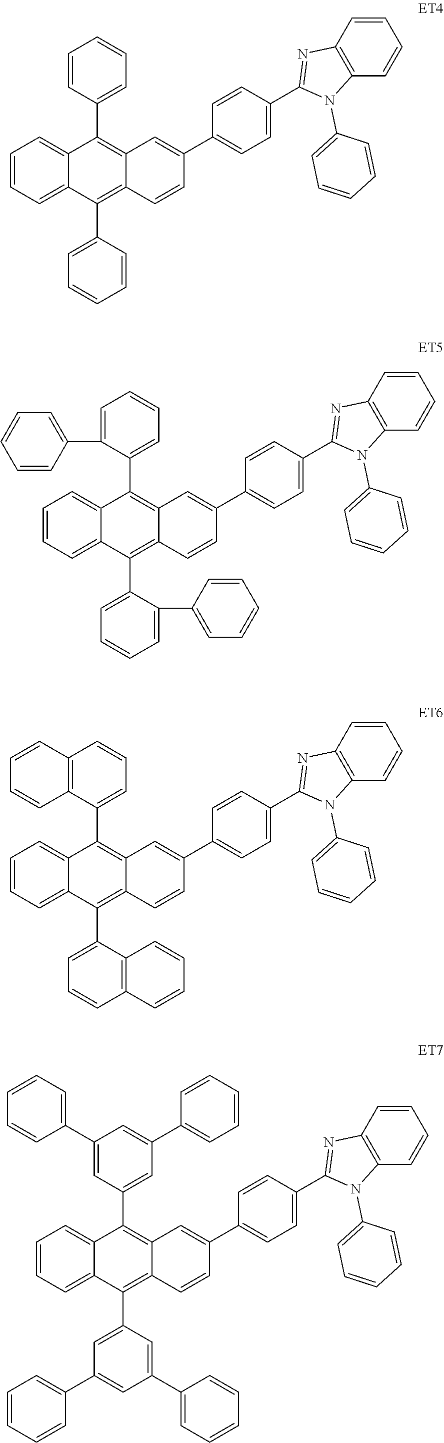 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

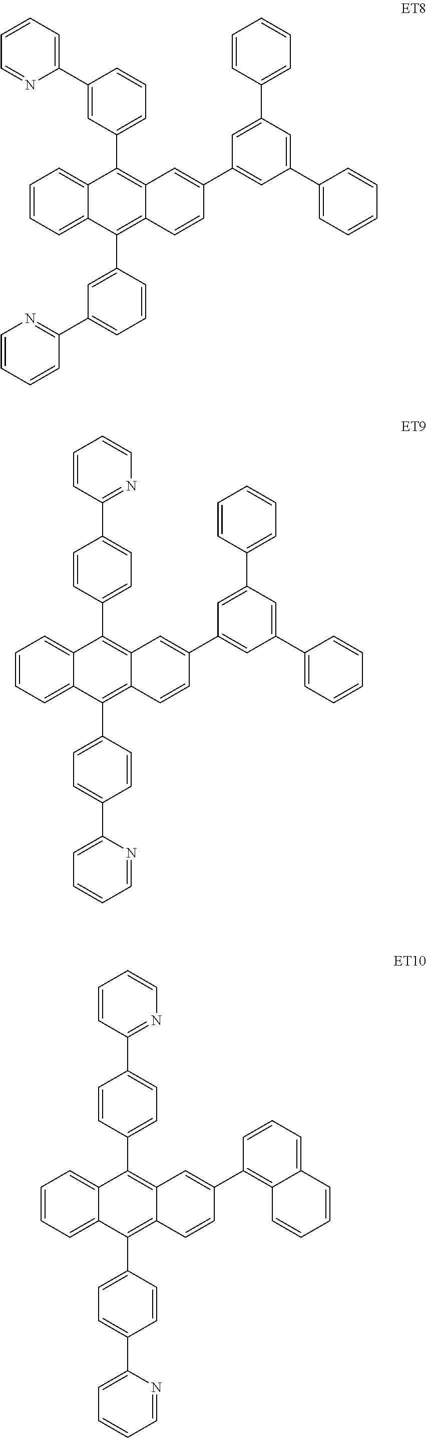 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

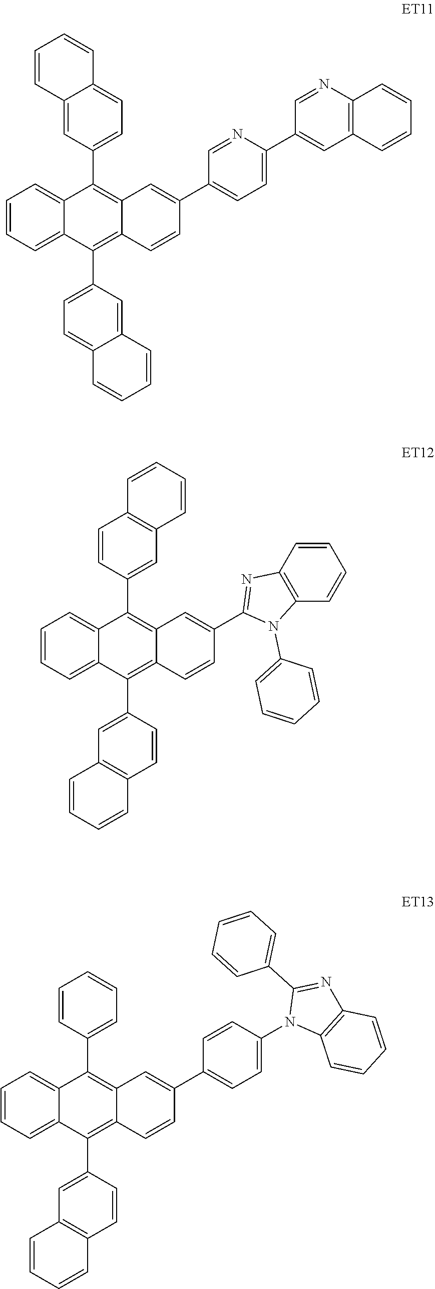 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

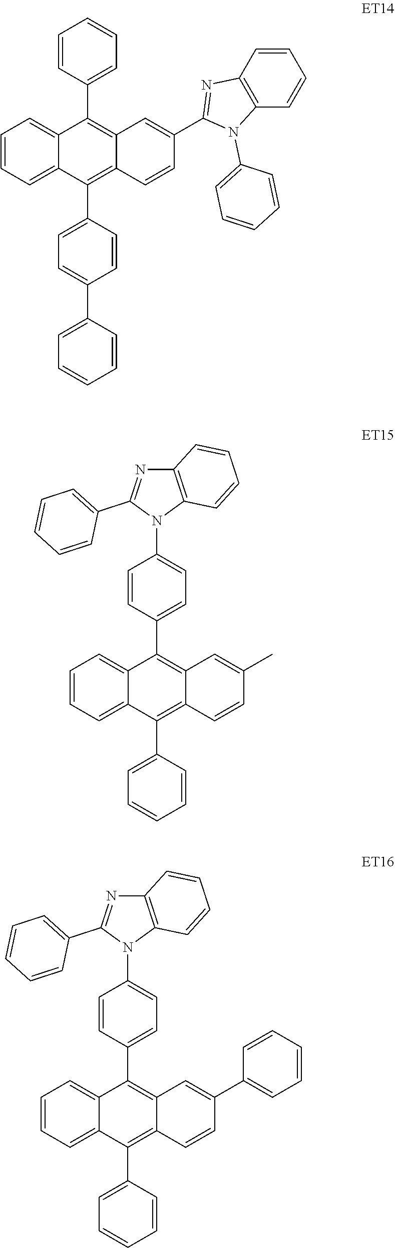 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

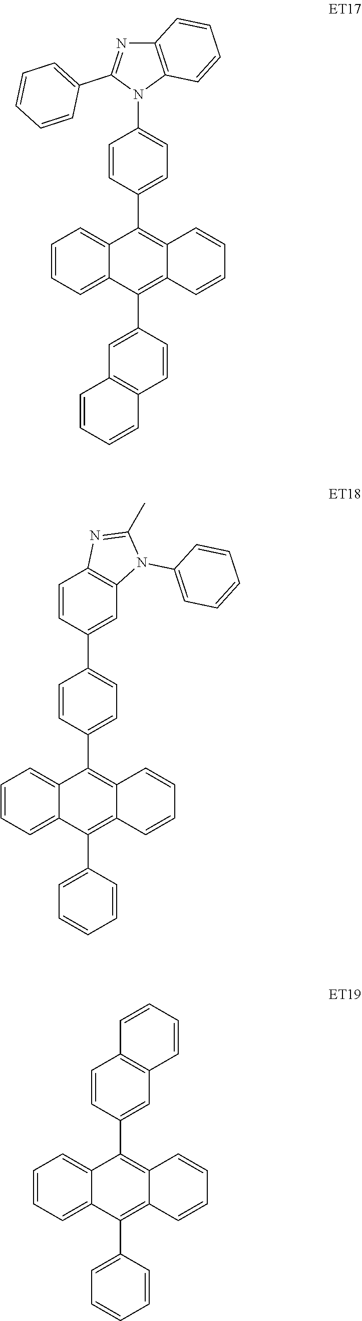

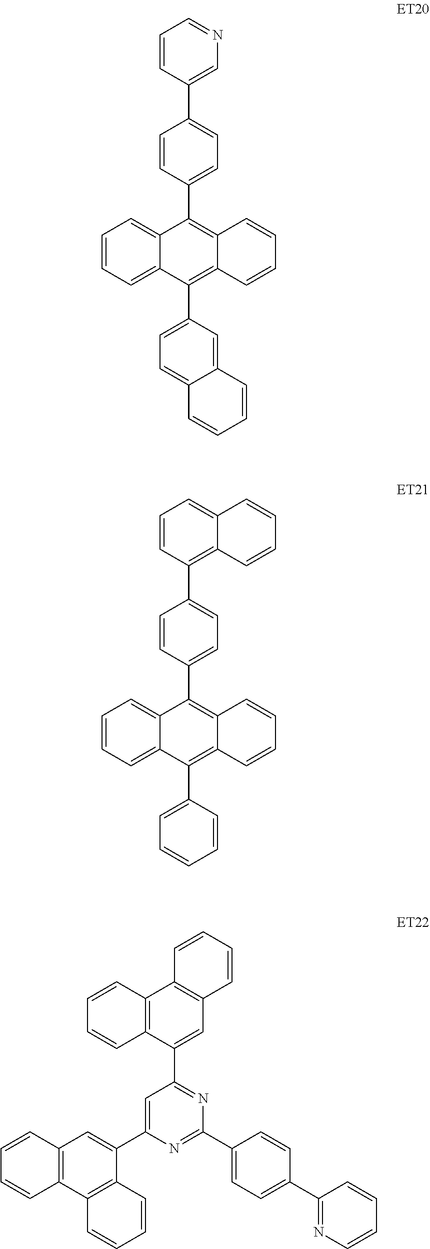

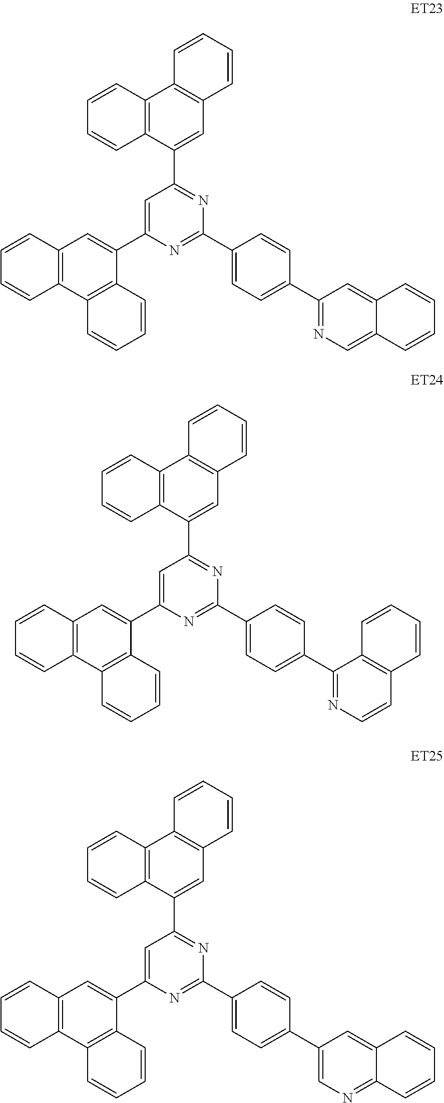 [SMILES: c1ccc2cc(-c3ccc(-c4nc(-c5cc6ccccc6c6ccccc56)cc(-c5cc6ccccc6c6ccccc56)n4)cc3)ncc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)cc(-c5cc6ccccc6c6ccccc56)n4)cc3)cc2c1]

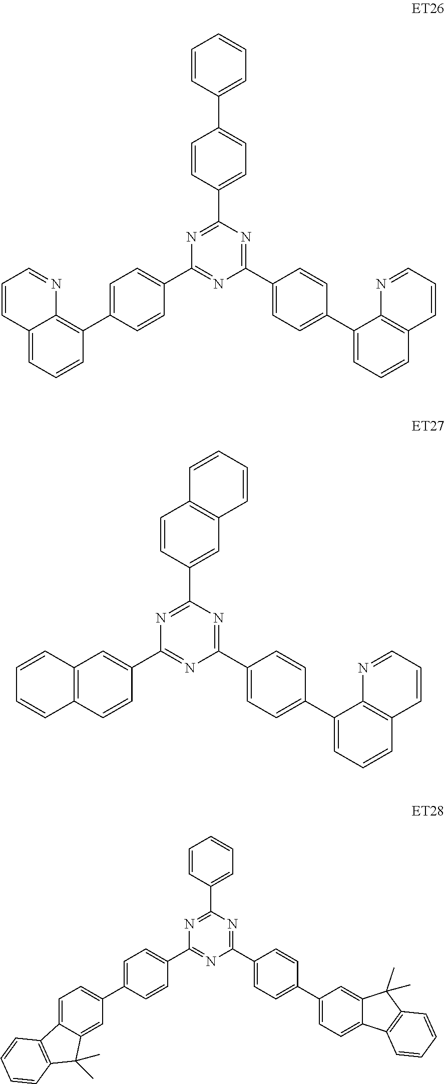 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21]

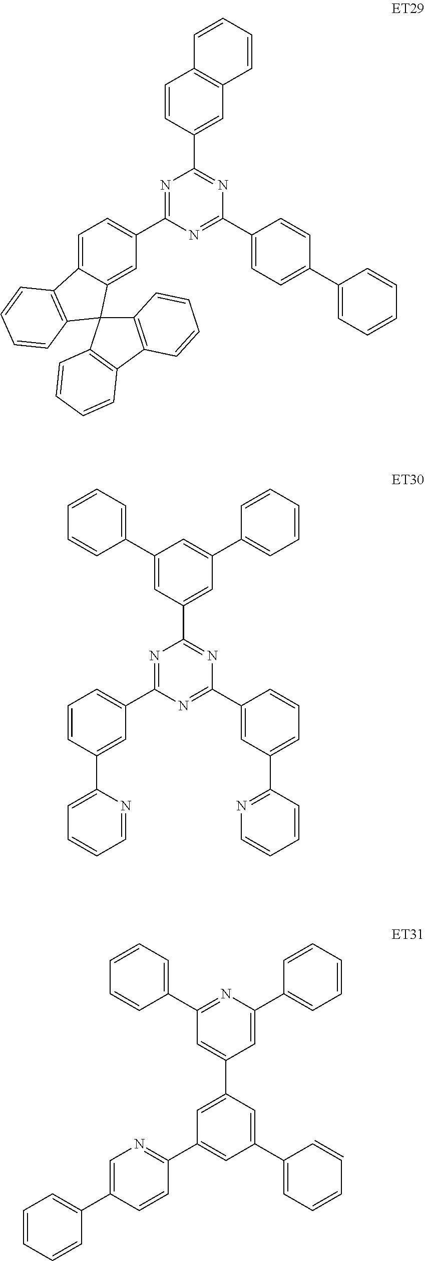 [SMILES: c1ccc(-c2ccc(-c3nc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4ccccc4)cc(-c4cc(-c5ccccc5)nc(-c5ccccc5)c4)c3)nc2)cc1]

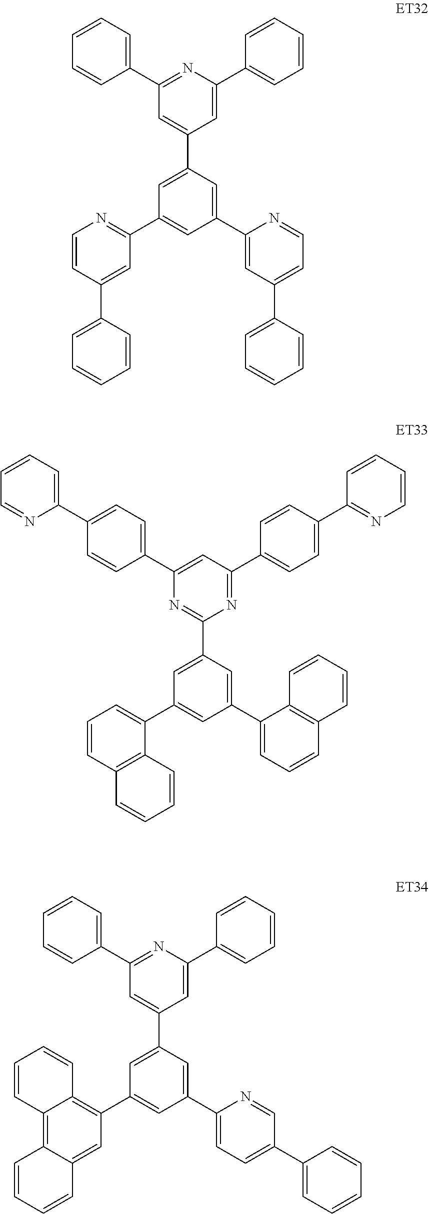 [SMILES: c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1]

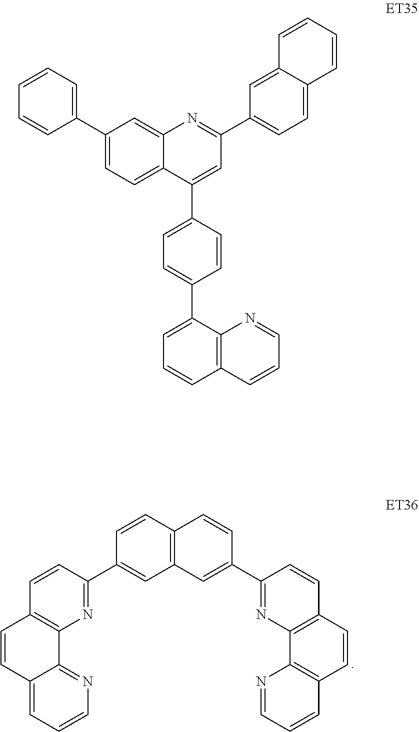 [SMILES: c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In some embodiments, the electron transport regions ETR may include a metal halide such as LiF, NaCl, CsF, RbCl, RbI, CuI, or KI, a lanthanide metal such as Yb, and a co-deposited material of the metal halide and the lanthanide metal. For example, the electron transport region ETR may include KI:Yb, RbI:Yb, LiF:Yb, etc. as a co-deposited material. In some embodiments, the electron transport region ETR may be formed utilizing a metal oxide such as Li₂O or BaO, or 8-hydroxyl-lithium quinolate (Liq), etc., but the embodiment of the present disclosure is not limited thereto. The electron transport region ETR may also be formed of a mixture material of an electron transport material and an insulating organometallic salt. The organometallic salt may be a material having an energy band gap of about 4 eV or more. For example, the organometallic salt may include, for example, a metal acetate, a metal benzoate, a metal acetoacetate, a metal acetylacetonate, or a metal stearate.

The electron transport region ETR may further include at least one of 2,9-dimethyl-4,7-diphenyl-1,10-phenanthroline (BCP), diphenyl(4-(triphenylsilyl)phenyl)phosphine oxide (TSPO1), or 4,7-diphenyl-1,10-phenanthroline (Bphen) in addition to the above-described materials, but the embodiment of the present disclosure is not limited thereto.

The electron transport region ETR may include the above-described compounds of the hole transport region in at least one of the electron injection layer EIL, the electron transport layer ETL, or the hole blocking layer HBL.

When the electron transport region ETR includes the electron transport layer ETL, the electron transport layer ETL may have a thickness of about 100 Å to about 1,000 Å, for example, about 150 Å to about 500 Å. When the thickness of the electron transport layer ETL satisfies the aforementioned range, satisfactory (suitable) electron transport characteristics may be obtained without a substantial increase in driving voltage. When the electron transport region ETR includes the electron injection layer EIL, the electron injection layer EIL may have a thickness of about 1 Å to about 100 Å, for example, about 3 Å to about 90 Å. When the thickness of the electron injection layer EIL satisfies the above-described range, satisfactory (suitable) electron injection characteristics may be obtained without a substantial increase in driving voltage.

The second electrode EL2 is provided on the electron transport region ETR. The second electrode EL2 may be a common electrode. The second electrode EL2 may be a cathode or an anode, but the embodiment of the present disclosure is not limited thereto. For example, when the first electrode EL1 is an anode, the second electrode EL2 may be a cathode, and when the first electrode EL1 is a cathode, the second electrode EL2 may be an anode. The second electrode EL2 may include at least one selected from among Ag, Mg, Cu, Al, Pt, Pd, Au, Ni, Nd, Ir, Cr, Li, Ca, LiF, Mo, Ti, W, In, Sn, Zn, one or more compounds thereof, one or more mixtures thereof, and/or oxides thereof.

The second electrode EL2 may be a transmissive electrode, a transflective electrode, or a reflective electrode. When the second electrode EL2 is the transmissive electrode, the second electrode EL2 may be formed of a transparent metal oxide, for example, indium tin oxide (ITO), indium zinc oxide (IZO), zinc oxide (ZnO), indium tin zinc oxide (ITZO), etc.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, Yb, W, or one or more compounds or mixtures including these (e.g., AgMg, AgYb, or MgYb). In some embodiments, the second electrode EL2 may have a multilayer structure including a reflective film or a transflective film formed of the above-described materials, and a transparent conductive film formed of ITO, IZO, ZnO, ITZO, etc. For example, the second electrode EL2 may include the above-described metal materials, combinations of at least two metal materials of the above-described metal materials, oxides of the above-described metal materials, and/or the like.

The second electrode EL2 may be connected with an auxiliary electrode. When the second electrode EL2 is connected with the auxiliary electrode, the resistance of the second electrode EL2 may be decreased.

In some embodiments, a capping layer CPL may further be disposed on the second electrode EL2 of the light emitting element ED of an embodiment. The capping layer CPL may include a multilayer or a single layer.

In an embodiment, the capping layer CPL may be an organic layer or an inorganic layer. For example, when the capping layer CPL contains an inorganic material, the inorganic material may include an alkaline metal compound (for example, LiF), an alkaline earth metal compound (for example, MgF₂), SiON, SiNₓ, SiOy, etc.

For example, when the capping layer CPL contains an organic material, the organic material may include α-NPD, NPB, TPD, m-MTDATA, Alq₃, CuPc, N4,N4,N4′,N4′-tetra(biphenyl-4-yl)biphenyl-4,4′-diamine (TPD15), 4,4′,4″-tris(N-carbazol yl)triphenylamine (TCTA), etc., or may include an epoxy resin, or an acrylate such as a methacrylate. However, the embodiment of the present disclosure is not limited thereto, and the capping layer CPL may include at least one selected from among Compounds P1 to P5:

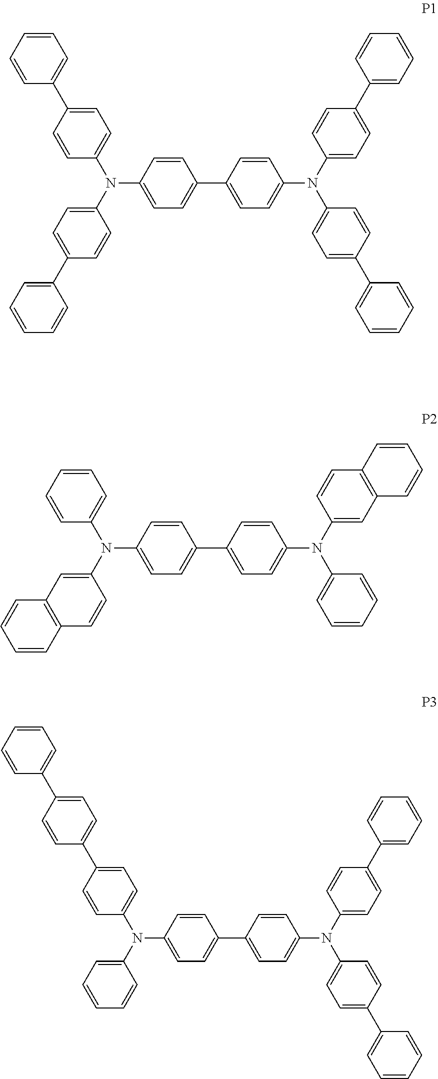 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

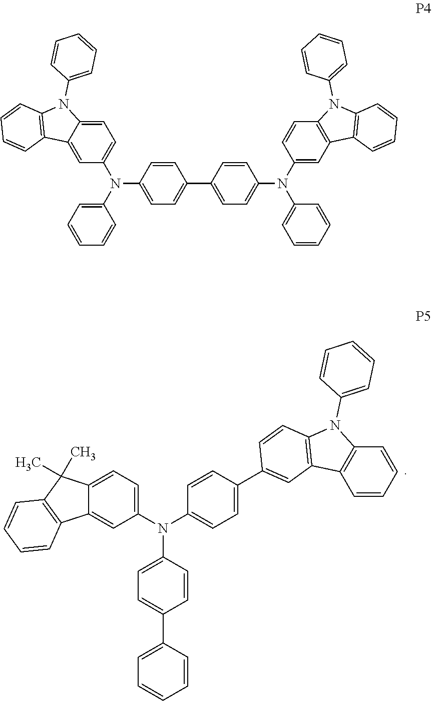 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In some embodiments, the refractive index of the capping layer CPL may be about 1.6 or more. For example, the refractive index of the capping layer CPL may be about 1.6 or more with respect to light in a wavelength range of about 550 nm to about 660 nm.

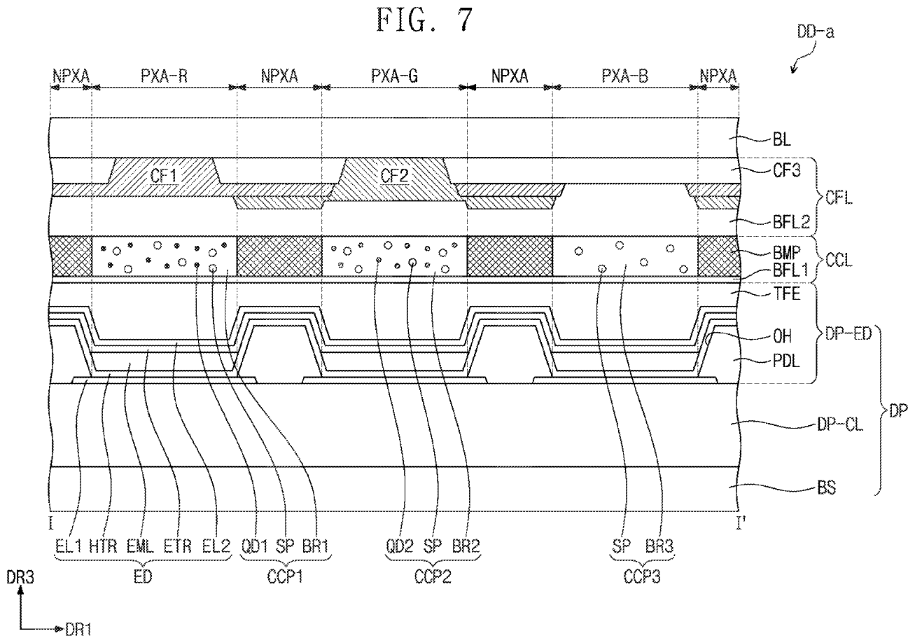

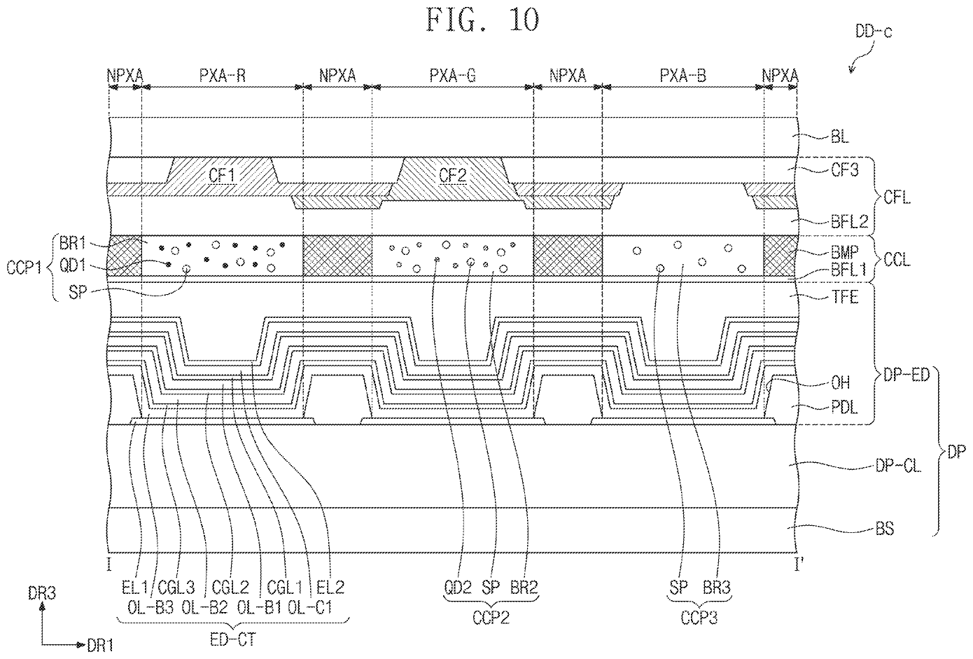

Each of FIGS. 7 to 10 is a cross-sectional view of a display device according to an embodiment of the present disclosure. Hereinafter, in describing the display devices of embodiments with reference to FIGS. 7 to 10, the duplicated features which have been described in FIGS. 1 to 6 may not be described again, but their differences will be mainly described.

Referring to FIG. 7, the display device DD-a according to an embodiment may include a display panel DP including a display element layer DP-ED, a light control layer CCL on the display panel DP, and a color filter layer CFL.

In an embodiment illustrated in FIG. 7, the display panel DP may include a base layer BS, a circuit layer DP-CL provided on the base layer BS, and the display element layer DP-ED, and the display element layer DP-ED may include a light emitting element ED.

The light emitting element ED may include a first electrode EL1, a hole transport region HTR on the first electrode EL1, an emission layer EML on the hole transport region HTR, an electron transport region ETR on the emission layer EML, and a second electrode EL2 on the electron transport region ETR. In some embodiments, the structures of the light emitting elements ED of FIGS. 3 to 6 as described above may be applied to the structure of the light emitting element ED illustrated in FIG. 7.

The hole transport region HTR of the light emitting element ED included in the display device DD-a according to an embodiment may include the above-described amine compound of an embodiment.

Referring to FIG. 7, the emission layer EML may be in an opening OH defined in a pixel defining film PDL. For example, the emission layer EML which is divided by the pixel defining film PDL and provided corresponding to each light emitting regions PXA-R, PXA-G, and PXA-B may be to emit light in substantially the same wavelength range. In the display device DD-a of an embodiment, the emission layer EML may emit blue light. In some embodiments, the emission layer EML may be provided as a common layer in the entire light emitting regions PXA-R, PXA-G, and PXA-B.

The light control layer CCL may be on the display panel DP. The light control layer CCL may include a light conversion body. The light conversion body may be a quantum dot, a phosphor, and/or the like. The light conversion body may emit provided light by converting the wavelength thereof. For example, the light control layer CCL may be a layer containing the quantum dot or a layer containing the phosphor.

The light control layer CCL may include a plurality of light control parts CCP1, CCP2 and CCP3. The light control parts CCP1, CCP2, and CCP3 may be spaced apart from each other.

Referring to FIG. 7, divided patterns BMP may be between the light control parts CCP1, CCP2 and CCP3 which are spaced apart from each other, but the embodiment of the present disclosure is not limited thereto. FIG. 7 illustrates that the divided patterns BMP do not overlap the light control parts CCP1, CCP2 and CCP3, but at least a portion of the edges of the light control parts CCP1, CCP2 and CCP3 may overlap the divided patterns BMP.

The light control layer CCL may include a first light control part CCP1 containing a first quantum dot QD1 which converts first color light provided from the light emitting element ED into second color light, a second light control part CCP2 containing a second quantum dot QD2 which converts the first color light into third color light, and a third light control part CCP3 which transmits the first color light.

In an embodiment, the first light control part CCP1 may provide red light that is the second color light, and the second light control part CCP2 may provide green light that is the third color light. The third light control part CCP3 may provide blue light by transmitting the blue light that is the first color light provided from the light emitting element ED. For example, the first quantum dot QD1 may be a red quantum dot, and the second quantum dot QD2 may be a green quantum dot. The same description used above may be applied with respect to the quantum dots QD1 and QD2.

In some embodiments, the light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include (e.g., may exclude) any quantum dot but include the scatterer SP.

The scatterer SP may be inorganic particles. For example, the scatterer SP may include at least one of TiO₂, ZnO, Al₂O₃, SiO₂, or hollow sphere silica. The scatterer SP may include any one selected from among TiO₂, ZnO, Al₂O₃, SiO₂, and hollow sphere silica, or may be a mixture of at least two materials selected from among TiO₂, ZnO, Al₂O₃, SiO₂, and hollow sphere silica.

The first light control part CCP1, the second light control part CCP2, and the third light control part CCP3 each may include base resins BR1, BR2, and BR3 in which the quantum dots QD1 and QD2 and the scatterer SP are dispersed. In an embodiment, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3. The base resins BR1, BR2, and BR3 are media in which the quantum dots QD1 and QD2 and the scatterer SP are dispersed, and may be formed of one or more suitable resin compositions, which may be generally referred to as a binder. For example, the base resins BR1, BR2, and BR3 may be acrylic-based resins, urethane-based resins, silicone-based resins, epoxy-based resins, etc. The base resins BR1, BR2, and BR3 may be transparent resins.

In an embodiment, the first base resin BR1, the second base resin BR2, and the third base resin BR3 may be the same as or different from each other.

The light control layer CCL may include a barrier layer BFL1. The barrier layer BFL1 may serve to prevent or reduce the penetration of moisture and/or oxygen (hereinafter, referred to as ‘moisture/oxygen’). The barrier layer BFL1 may be on the light control parts CCP1, CCP2, and CCP3 to block or reduce the light control parts CCP1, CCP2 and CCP3 from being exposed to moisture/oxygen. In some embodiments, the barrier layer BFL1 may cover the light control parts CCP1, CCP2, and CCP3. In some embodiments, the barrier layer BFL2 may be provided between the light control parts CCP1, CCP2, and CCP3 and the color filter layer CFL.

The barrier layers BFL1 and BFL2 may include at least one inorganic layer. For example, the barrier layers BFL1 and BFL2 may include an inorganic material. For example, the barrier layers BFL1 and BFL2 may include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film which secures a transmittance, etc. In some embodiments, the barrier layers BFL1 and BFL2 may further include an organic film. The barrier layers BFL1 and BFL2 may be formed of a single layer or a plurality of layers.

In the display device DD-a of an embodiment, the color filter layer CFL may be on the light control layer CCL. For example, the color filter layer CFL may be directly on the light control layer CCL. In this embodiment, the barrier layer BFL2 may not be provided.

The color filter layer CFL may include filters CF1, CF2, and CF3. The color filter layer CFL may include a first filter CF1 configured to transmit the second color light, a second filter CF2 configured to transmit the third color light, and a third filter CF3 configured to transmit the first color light. For example, the first filter CF1 may be a red filter, the second filter CF2 may be a green filter, and the third filter CF3 may be a blue filter. The filters CF1, CF2, and CF3 may each include a polymeric photosensitive resin and a pigment or dye. The first filter CF1 may include a red pigment or dye, the second filter CF2 may include a green pigment or dye, and the third filter CF3 may include a blue pigment or dye. However, the embodiment of the present disclosure is not limited thereto, and the third filter CF3 may not include (e.g., may exclude) a pigment or dye. The third filter CF3 may include a polymeric photosensitive resin and may not include (e.g., may exclude) a pigment or dye. The third filter CF3 may be transparent. The third filter CF3 may be formed of a transparent photosensitive resin.

Furthermore, in an embodiment, the first filter CF1 and the second filter CF2 may be a yellow filter. The first filter CF1 and the second filter CF2 may not be separated but be provided as one filter. The first to third filters CF1, CF2, and CF3 may be disposed corresponding to the red light emitting region PXA-R, the green light emitting region PXA-G, and the blue light emitting region PXA-B, respectively.

In some embodiments, the color filter layer CFL may include a light shielding part. The color filter layer CFL may include a light shielding part disposed to overlap at the boundaries of neighboring filters CF1, CF2, and CF3. The light shielding part may be a black matrix. The light shielding part may include an organic light shielding material or an inorganic light shielding material containing a black pigment or dye. The light shielding part may separate boundaries between the adjacent filters CF1, CF2, and CF3. In some embodiments, in an embodiment, the light shielding part may be formed of a blue filter.

A base substrate BL may be on the color filter layer CFL. The base substrate BL may be a member which provides a base surface in which the color filter layer CFL, the light control layer CCL, and/or the like are disposed. The base substrate BL may be a glass substrate, a metal substrate, a plastic substrate, etc. However, the embodiment of the present disclosure is not limited thereto, and the base substrate BL may be an inorganic layer, an organic layer, or a composite material layer. In some embodiments, the base substrate BL may not be provided.

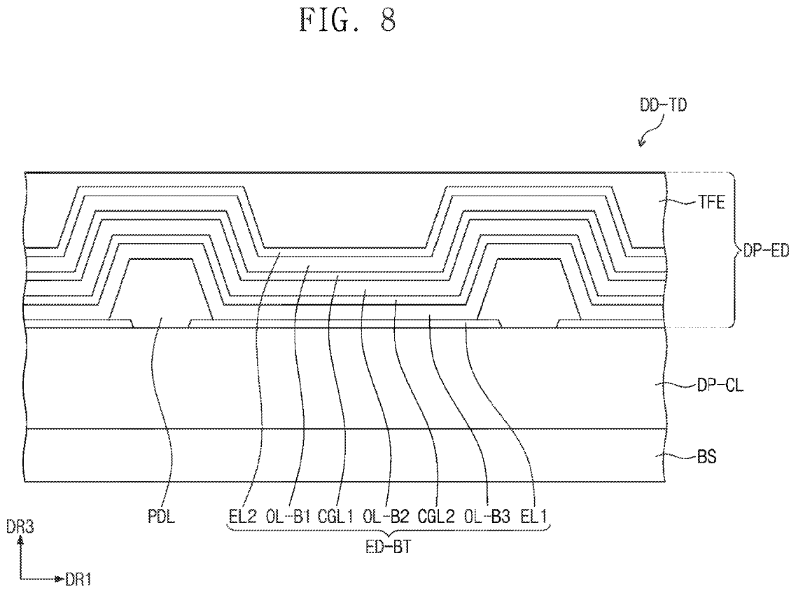

FIG. 8 is a cross-sectional view illustrating a portion of a display device according to an embodiment of the present disclosure. FIG. 8 illustrates a cross-sectional view of a part corresponding to the display panel DP of FIG. 7. In the display device DD-TD of an embodiment, the light emitting element ED-BT may include a plurality of light emitting structures OL-B1, OL-B2, and OL-B3. The light emitting element ED-BT may include a first electrode EL1 and a second electrode EL2 which face each other, and the plurality of light emitting structures OL-B1, OL-B2, and OL-B3 sequentially stacked in the thickness direction between the first electrode EL1 and the second electrode EL2. The light emitting structures OL-B1, OL-B2, and OL-B3 each may include an emission layer EML (FIG. 7) and a hole transport region HTR and an electron transport region ETR disposed with the emission layer EML (FIG. 7) located therebetween.

For example, the light emitting element ED-BT included in the display device DD-TD of an embodiment may be a light emitting element having a tandem structure and including a plurality of emission layers EML.

In an embodiment illustrated in FIG. 8, all light beams respectively emitted from the light emitting structures OL-B1, OL-B2, and OL-B3 may be blue light. However, the embodiment of the present disclosure is not limited thereto, and the light beams respectively emitted from the light emitting structures OL-B1, OL-B2, and OL-B3 may have wavelength ranges different from each other. For example, the light emitting element ED-BT including the plurality of light emitting structures OL-B1, OL-B2, and OL-B3 which emit light beams having wavelength ranges different from each other may emit white light.

Charge generation layers CGL1 and CGL2 may be respectively disposed between two of the neighboring light emitting structures OL-B1, OL-B2, and OL-B3. The charge generation layers CGL1 and CGL2 may include a p-type or kind charge generation layer and/or an n-type or kind charge generation layer.

At least one of the light emitting structures OL-B1, OL-B2, or OL-B3 included in the display device DD-TD of an embodiment may contain the above-described amine compound of an embodiment.

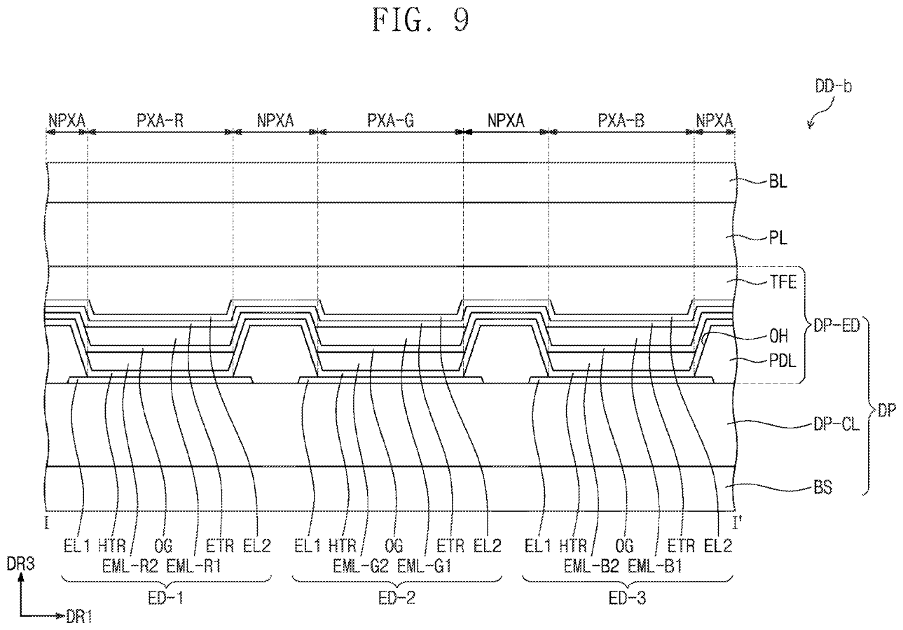

Referring to FIG. 9, the display device DD-b according to an embodiment may include light emitting elements ED-1, ED-2, and ED-3 in which two emission layers are stacked. Compared with the display device DD of an embodiment illustrated in FIG. 2, an embodiment illustrated in FIG. 9 has a difference in that the first to third light emitting elements ED-1, ED-2, and ED-3 each include two emission layers stacked in the thickness direction. In each of the first to third light emitting elements ED-1, ED-2, and ED-3, the two emission layers may emit light in substantially the same wavelength region.

The first light emitting element ED-1 may include a first red emission layer EML-R1 and a second red emission layer EML-R2. The second light emitting element ED-2 may include a first green emission layer EML-G1 and a second green emission layer EML-G2. In some embodiments, the third light emitting element ED-3 may include a first blue emission layer EML-B1 and a second blue emission layer EML-B2. An emission auxiliary part OG may be between the first red emission layer EML-R1 and the second red emission layer EML-R2, between the first green emission layer EML-G1 and the second green emission layer EML-G2, and between the first blue emission layer EML-B1 and the second blue emission layer EML-B2.

The emission auxiliary part OG may include a single layer or a multilayer. The emission auxiliary part OG may include a charge generation layer. For example, the emission auxiliary part OG may include an electron transport region, a charge generation layer, and a hole transport region that are sequentially stacked. The emission auxiliary part OG may be provided as a common layer in the whole of the first to third light emitting elements ED-1, ED-2, and ED-3. However, the embodiment of the present disclosure is not limited thereto, and the emission auxiliary part OG may be provided by being patterned within the openings OH defined in the pixel defining film PDL.

The first red emission layer EML-R1, the first green emission layer EML-G1, and the first blue emission layer EML-B1 may be between the electron transport region ETR and the emission auxiliary part OG. The second red emission layer EML-R2, the second green emission layer EML-G2, and the second blue emission layer EML-B2 may be between the emission auxiliary part OG and the hole transport region HTR.

For example, the first light emitting element ED-1 may include the first electrode EL1, the hole transport region HTR, the second red emission layer EML-R2, the emission auxiliary part OG, the first red emission layer EML-R1, the electron transport region ETR, and the second electrode EL2 that are sequentially stacked. The second light emitting element ED-2 may include the first electrode EL1, the hole transport region HTR, the second green emission layer EML-G2, the emission auxiliary part OG, the first green emission layer EML-G1, the electron transport region ETR, and the second electrode EL2 that are sequentially stacked. The third light emitting element ED-3 may include the first electrode EL1, the hole transport region HTR, the second blue emission layer EML-B2, the emission auxiliary part OG, the first blue emission layer EML-B1, the electron transport region ETR, and the second electrode EL2 that are sequentially stacked.

In some embodiments, an optical auxiliary layer PL may be on the display element layer DP-ED. The optical auxiliary layer PL may include a polarizing layer. The optical auxiliary layer PL may be on the display panel DP and control reflected light in the display panel DP due to external light. Unlike the configuration illustrated, the optical auxiliary layer PL in the display device according to an embodiment may not be provided.

Unlike FIGS. 8 and 9, FIG. 10 illustrates that a display device DD-c includes four light emitting structures OL-B1, OL-B2, OL-B3, and OL-C1. A light emitting element ED-CT may include a first electrode EL1 and a second electrode EL2 which face each other, and first to fourth light emitting structures OL-B1, OL-B2, OL-B3, and OL-C1 that are sequentially stacked in the thickness direction between the first electrode EL1 and the second electrode EL2. Charge generation layers CGL1, CGL2, and CGL3 may be between the first to fourth light emitting structures OL-B1, OL-B2, OL-B3, and OL-C1. Among the four light emitting structures, the first to third light emitting structures OL-B1, OL-B2, and OL-B3 may be to emit blue light, and the fourth light emitting structure OL-C1 may be to emit green light. However, the embodiment of the present disclosure is not limited thereto, and the first to fourth light emitting structures OL-B1, OL-B2, OL-B3, and OL-C1 may be to emit light beams in different wavelength regions.

The charge generation layers CGL1, CGL2, and CGL3 disposed between adjacent light emitting structures OL-B1, OL-B2, OL-B3, and OL-C1 may include a p-type or kind charge generation layer and/or an n-type or kind charge generation layer.

At least one among the light emitting structures OL-B1, OL-B2, OL-B3, and OL-C1 included in the display device DD-c of an embodiment may include the above-described amine compound of an embodiment.

The light emitting element ED according to an embodiment of the present disclosure may include the above-described amine compound of an embodiment in at least one functional layer between the first electrode EL1 and the second electrode EL2, thereby exhibiting improved luminous efficiency and service life characteristics. The light emitting element ED according to an embodiment may include the above-described amine compound of an embodiment in at least one of the hole transport region HTR, the emission layer EML, or the electron transport region ETR between the first electrode EL1 and the second electrode EL2, or in a capping layer CPL.

For example, the amine compound according to an embodiment may be included in the hole transport region HTR of the light emitting element ED of an embodiment, and the light emitting element of an embodiment may exhibit excellent or suitable luminous efficiency and long service life characteristics.

For the above-described amine compound of an embodiment, the carbazole moiety is bonded to the amine moiety at the second position of the carbazole, and two dibenzoheterole moieties are bonded via a specific linker to the nitrogen atom of the amine or directly bonded at a specific position, thereby exhibiting high efficiency and increased service life characteristics.

Hereinafter, with reference to Examples and Comparative Examples, an amine compound according to an embodiment of the present disclosure and a light emitting element of an embodiment of the present disclosure will be described in more detail. In some embodiments, examples described are merely illustrations to assist the understanding of the present disclosure, and the scope of the present disclosure is not limited thereto.

### Examples

### 1. Synthesis of Amine Compound

First, a synthetic method of an amine compound according to the present embodiment will be described in more detail by illustrating the synthetic methods of Compounds A2, A17, A69, A90, B4, B36, B43, B86, C38, C61, C76, C79, D19, D35, D54, D81, A39, B40, and C83. Also, in the following descriptions, the synthetic method of the amine compound is provided as an example, but the synthetic method according to an embodiment of the present disclosure is not limited to Examples.

### (1) Synthesis of Compound A2

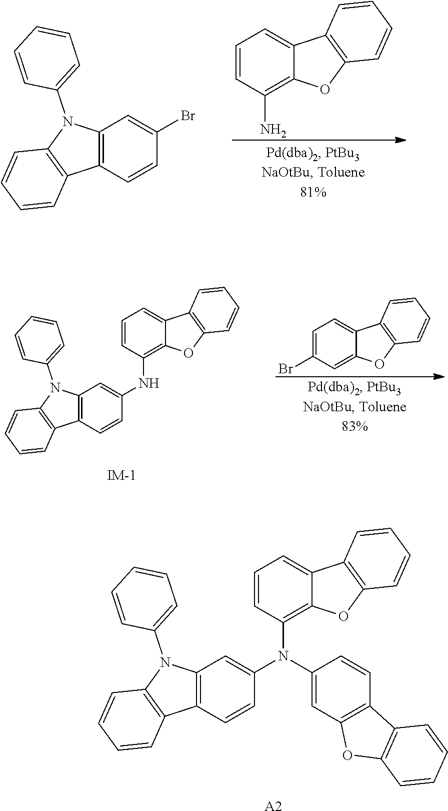

### 1) Synthesis of Intermediate Compound IM-1

In an argon (Ar) atmosphere, in a 500 mL three-neck flask, dibenzofuran-4-amine (10.00 g, 54.6 mmol), Pd(dba)₂ (0.94 g, 0.03 equiv, 1.6 mmol), NaOtBu (5.25 g, 1.0 equiv, 54.5 mmol), toluene (272 mL), 2-bromo-9-phenyl-9H-carbazole (19.25 g, 1.1 equiv, 60.0 mmol), and PtBu₃ (1.10 g, 0.1 equiv, 7.2 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-1 (18.77 g, yield 81%).

By measuring FAB-MS, a mass number of m/z=424 was observed by molecular ion peak, thereby identifying Intermediate Compound IM-1.

### 2) Synthesis of Compound A2

In an Ar atmosphere, in a 300 mL three-neck flask, Intermediate IM-1 (10.00 g, 23.6 mmol), Pd(dba)₂ (0.41 g, 0.03 equiv, 0.7 mmol), NaOtBu (4.53 g, 2.0 equiv, 47.1 mmol), toluene (118 mL), 3-bromodibenzofuran (6.40 g, 1.1 equiv, 25.9 mmol), and PtBu₃ (0.48 g, 0.1 equiv, 2.4 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain solid Compound A2 (11.55 g, yield 83%).

By measuring FAB-MS, a mass number of m/z=590 was observed by molecular ion peak, thereby identifying Compound A2.

### (2) Synthesis of Compound A17

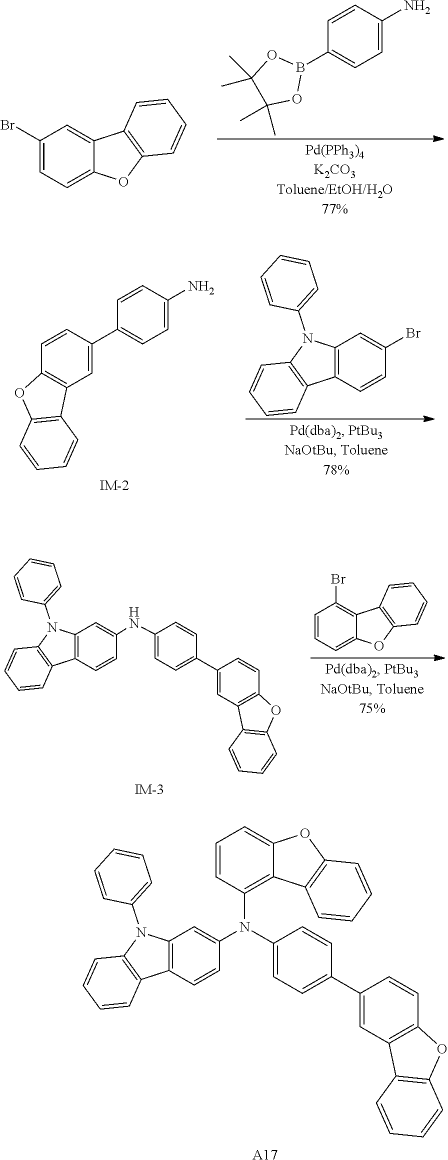

### 1) Synthesis of Intermediate Compound IM-2

In an Ar atmosphere, in a 1,000 mL three-neck flask, 2-bromodibenzofuran (20.00 g, 80.9 mmol), 4-(4,4,5,5-tetramethyl-1,3,2-dioxaborolan-2-yl)aniline (19.51 g, 1.1 equiv, 89.0 mmol), K₂CO₃ (33.56 g, 3.0 equiv, 242.8 mmol), Pd(PPh₃)₄ (4.68 g, 0.05 eq, 4.0 mmol), and a mixed solution of toluene/EtOH/H₂O (volume ratio=4/2/1) (567 mL) were sequentially added, and heated and stirred at about 80° C. After air-cooled to room temperature, the reaction solution was extracted with toluene. A water layer was removed, and organic layers were washed with saturated saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-2 (16.16 g, yield 77%).

By measuring FAB-MS, a mass number of m/z=259 was observed by molecular ion peak, thereby identifying Intermediate Compound IM-2.

### 2) Synthesis of Intermediate Compound IM-3

In an Ar atmosphere, in a 500 mL three-neck flask, Intermediate Compound IM-2 (10.00 g, 38.6 mmol), Pd(dba)₂ (0.67 g, 0.03 equiv, 1.2 mmol), NaOtBu (3.71 g, 1.0 equiv, 38.6 mmol), toluene (192 mL), 2-bromo-9-phenyl-9H-carbazole (13.67 g, 1.1 equiv, 42.4 mmol), and PtBu₃ (0.78 g, 0.1 equiv, 3.9 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-3 (15.06 g, yield 78%).

By measuring FAB-MS, a mass number of m/z=500 was observed by molecular ion peak, thereby identifying Intermediate Compound IM-3.

### 3) Synthesis of Compound A17

In an Ar atmosphere, in a 300 mL three-neck flask, Intermediate Compound IM-3 (10.00 g, 20.0 mmol), Pd(dba)₂ (0.34 g, 0.03 equiv, 0.6 mmol), NaOtBu (3.84 g, 2.0 equiv, 40.0 mmol), toluene (100 mL), 1-bromodibenzofuran (5.43 g, 1.1 equiv, 22.0 mmol), and PtBu₃ (0.40 g, 0.1 equiv, 2.0 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain solid Compound A17 (9.99 g, yield 75%).

By measuring FAB-MS, a mass number of m/z=666 was observed by molecular ion peak, thereby identifying Compound A17.

### (3) Synthesis of Compound A69

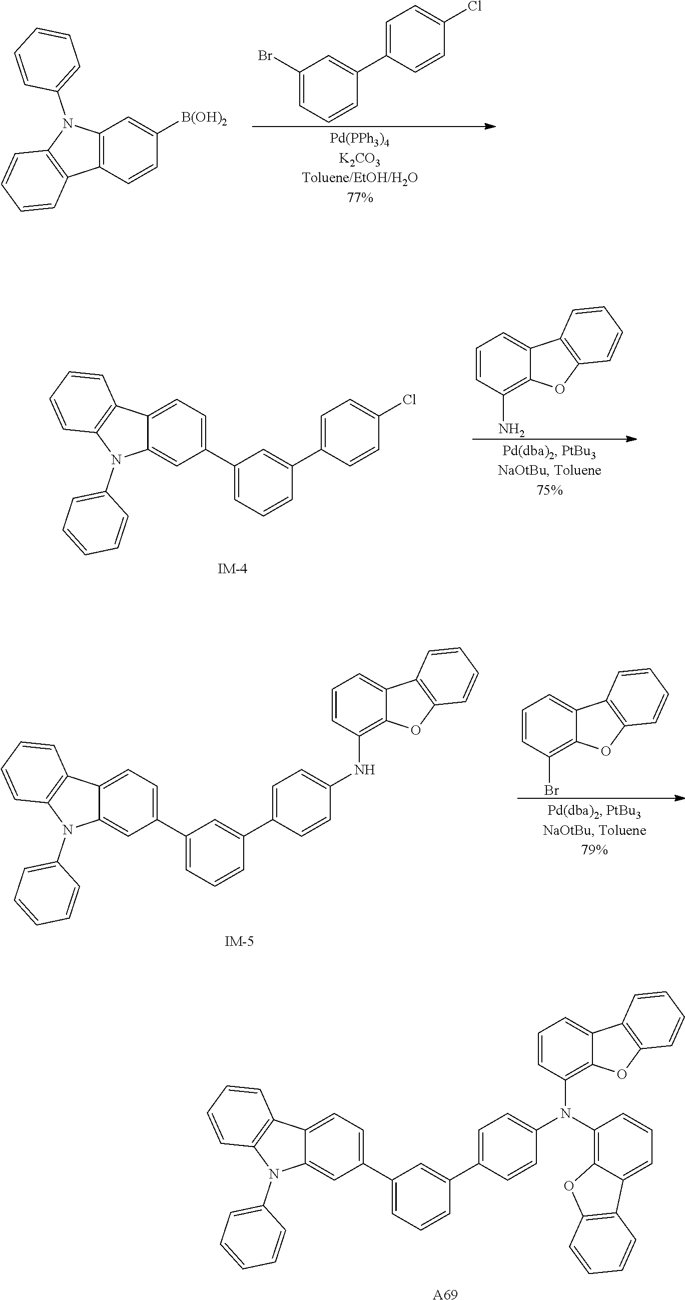

### 1) Synthesis of Intermediate Compound IM-4

In an Ar atmosphere, in a 1,000 mL three-neck flask, 3-bromo-4′-chloro-1,1′-biphenyl (20.00 g, 74.8 mmol), (9-phenyl-9H-carbazol-2-yl)boronic acid (23.61 g, 1.1 equiv, 82.2 mmol), K₂CO₃ (31.00 g, 3.0 equiv, 224.3 mmol), Pd(PPh₃)₄ (4.32 g, 0.05 eq, 3.7 mmol), and a mixed solution of toluene/EtOH/H₂O (volume ratio=4/2/1) (523 mL) were sequentially added, and heated and stirred at about 80° C. After air-cooled to room temperature, the reaction solution was extracted with toluene. A water layer was removed, and organic layers were washed with saturated saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-4 (24.75 g, yield 77%).

By measuring FAB-MS, a mass number of m/z=429 was observed by molecular ion peak, thereby identifying Intermediate Compound IM-4.

### 2) Synthesis of Intermediate Compound IM-5

In an Ar atmosphere, in a 500 mL three-neck flask, dibenzofuran-4-amine (10.00 g, 54.6 mmol), Pd(dba)₂ (0.94 g, 0.03 equiv, 1.6 mmol), NaOtBu (5.25 g, 1.0 equiv, 54.6 mmol), toluene (273 mL), Intermediate Compound IM-4 (25.81 g, 1.1 equiv, 60.0 mmol), and PtBu₃ (1.10 g, 0.1 equiv, 5.5 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-5 (23.61 g, yield 75%).

By measuring FAB-MS, a mass number of m/z=576 was observed by molecular ion peak, thereby identifying Intermediate Compound IM-5.

### 3) Synthesis of Compound A69

In an Ar atmosphere, in a 300 mL three-neck flask, Intermediate Compound IM-5 (10.00 g, 17.3 mmol), Pd(dba)₂ (0.30 g, 0.03 equiv, 0.5 mmol), NaOtBu (3.33 g, 2.0 equiv, 34.7 mmol), toluene (87 mL), 4-bromodibenzofuran (4.71 g, 1.1 equiv, 19.1 mmol), and PtBu₃ (0.35 g, 0.1 equiv, 1.7 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain solid Compound A69 (10.18 g, yield 79%).

By measuring FAB-MS, a mass number of m/z=742 was observed by molecular ion peak, thereby identifying Compound A69.

### (4) Synthesis of Compound A90

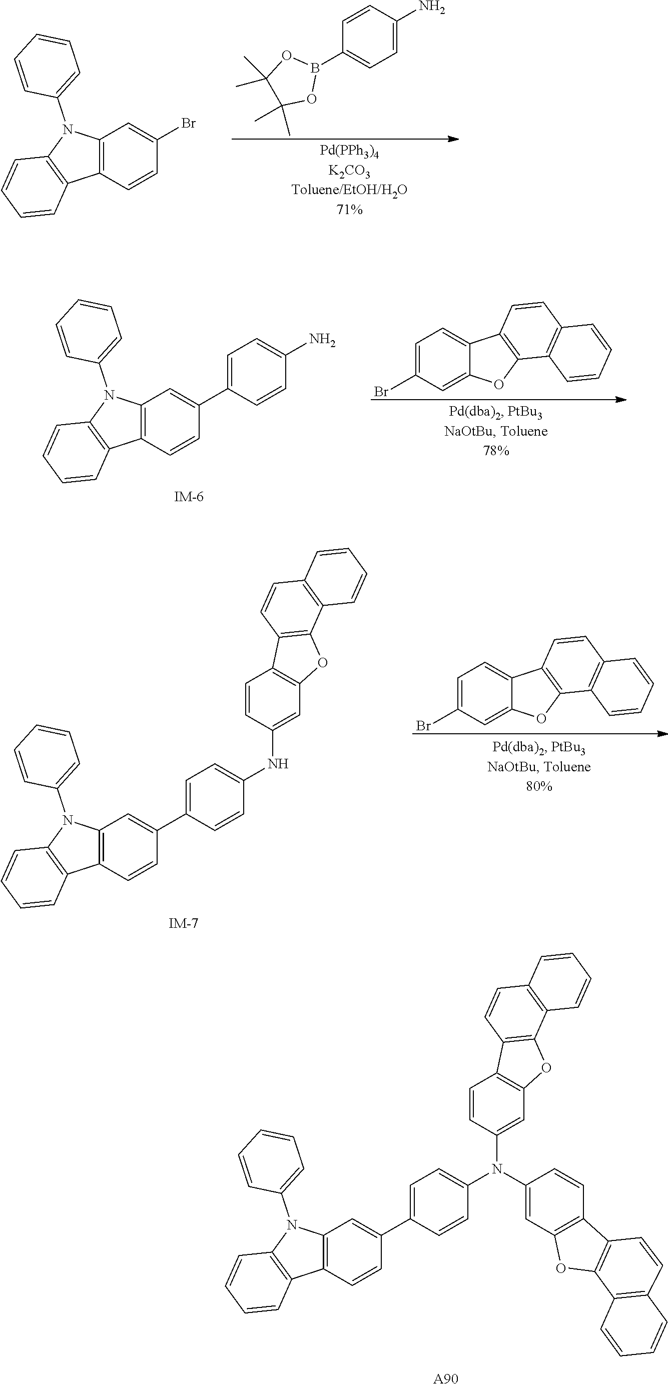

### 1) Synthesis of Intermediate Compound IM-6

In an Ar atmosphere, in a 1,000 mL three-neck flask, 2-bromo-9-phenyl-9H-carbazole (20.00 g, 62.1 mmol), 4-(4,4,5,5-tetramethyl-1,3,2-dioxaborolan-2-yl)aniline (14.96 g, 1.1 equiv, 68.3 mmol), K₂CO₃ (25.74 g, 3.0 equiv, 186.2 mmol), Pd(PPh₃)₄ (3.59 g, 0.05 eq, 3.1 mmol), and a mixed solution of toluene/EtOH/H₂O (volume ratio=4/2/1) (434 mL) were sequentially added, and heated and stirred at about 80° C. After air-cooled to room temperature, the reaction solution was extracted with toluene. A water layer was removed, and organic layers were washed with saturated saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-6 (14.74 g, yield 71%).

By measuring FAB-MS, a mass number of m/z=334 was observed by molecular ion peak, thereby identifying Intermediate Compound IM-6.

### 2) Synthesis of Intermediate Compound IM-7

In an Ar atmosphere, in a 500 mL three-neck flask, Intermediate Compound IM-6 (10.00 g, 29.9 mmol), Pd(dba)₂ (0.52 g, 0.03 equiv, 0.9 mmol), NaOtBu (2.87 g, 1.0 equiv, 29.9 mmol), toluene (150 mL), 9-bromonaphtho[1,2-b]benzofuran (9.77 g, 1.1 equiv, 32.9 mmol), and PtBu₃ (0.60 g, 0.1 equiv, 3.0 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-7 (12.84 g, yield 78%).

By measuring FAB-MS, a mass number of m/z=550 was observed by molecular ion peak, thereby identifying Intermediate Compound IM-7.

### 3) Synthesis of Compound A90

In an Ar atmosphere, in a 300 mL three-neck flask, Intermediate Compound IM-7 (10.00 g, 18.2 mmol), Pd(dba)₂ (0.31 g, 0.03 equiv, 0.5 mmol), NaOtBu (3.49 g, 2.0 equiv, 36.3 mmol), toluene (91 mL), 9-bromonaphtho[1,2-b]benzofuran (5.94 g, 1.1 equiv, 20.0 mmol), and PtBu₃ (0.37 g, 0.1 equiv, 1.8 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain solid Compound 90 (11.14 g, yield 80%).

By measuring FAB-MS, a mass number of m/z=766 was observed by molecular ion peak, thereby identifying Compound A90.

### (5) Synthesis of Compound B4

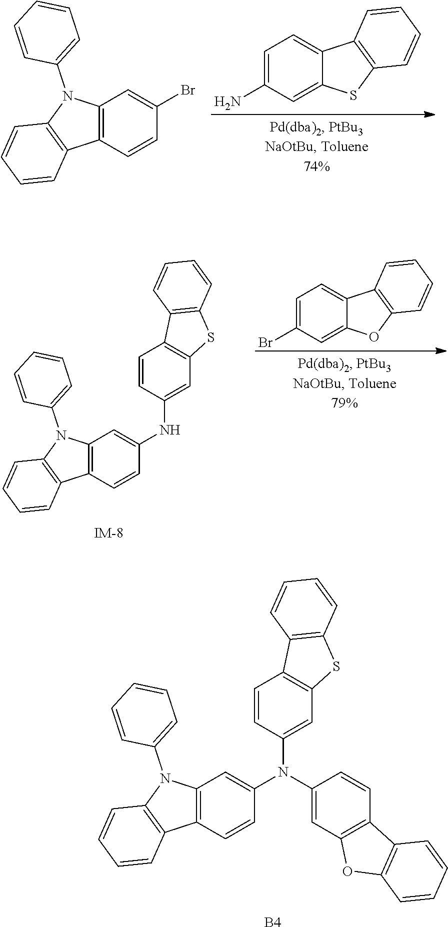

### 1) Synthesis of Intermediate Compound IM-8

In an Ar atmosphere, in a 500 mL three-neck flask, dibenzothiophen-3-amine (10.00 g, 50.2 mmol), Pd(dba)₂ (0.87 g, 0.03 equiv, 1.5 mmol), NaOtBu (4.82 g, 1.0 equiv, 50.2 mmol), toluene (250 mL), 2-bromo-9-phenyl-9H-carbazole (17.79 g, 1.1 equiv, 55.2 mmol), and PtBu₃ (1.02 g, 0.1 equiv, 5.0 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-8 (16.36 g, yield 74%).

By measuring FAB-MS, a mass number of m/z=440 was observed by molecular ion peak, thereby identifying Intermediate Compound IM-8.

### 2) Synthesis of Compound B4

In an Ar atmosphere, in a 300 mL three-neck flask, Intermediate Compound IM-8 (10.00 g, 22.7 mmol), Pd(dba)₂ (0.39 g, 0.03 equiv, 0.7 mmol), NaOtBu (4.36 g, 2.0 equiv, 45.4 mmol), toluene (113 mL), 3-bromodibenzofuran (6.17 g, 1.1 equiv, 25.0 mmol), and PtBu₃ (0.46 g, 0.1 equiv, 2.3 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain solid Compound B4 (10.88 g, yield 79%).

By measuring FAB-MS, a mass number of m/z=606 was observed by molecular ion peak, thereby identifying Compound B4.

### (6) Synthesis of Compound B36

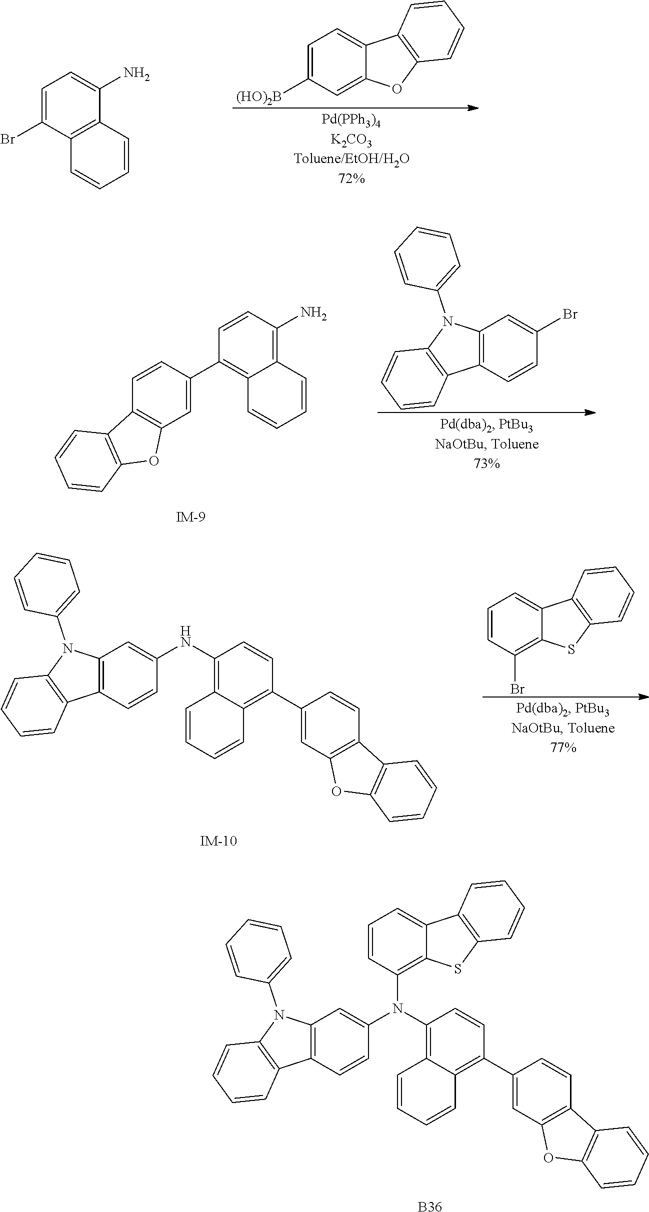

### 1) Synthesis of Intermediate Compound IM-9

In an Ar atmosphere, in a 1,000 mL three-neck flask, 4-bromonaphthalen-1-amine (20.00 g, 90.1 mmol), dibenzofuran-3-boronic acid (21.00 g, 1.1 equiv, 99.1 mmol), K₂C03 (37.34 g, 3.0 equiv, 270.2 mmol), Pd(PPh₃)₄ (5.20 g, 0.05 eq, 4.5 mmol), and a mixed solution of toluene/EtOH/H₂O (volume ratio=4/2/1) (434 mL) were sequentially added, and heated and stirred at about 80° C. After air-cooled to room temperature, the reaction solution was extracted with toluene. A water layer was removed, and organic layers were washed with saturated saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-9 (20.06 g, yield 72%).

By measuring FAB-MS, a mass number of m/z=309 was observed by molecular ion peak, thereby identifying Intermediate Compound IM-9.

### 2) Synthesis of Intermediate Compound IM-10

In an Ar atmosphere, in a 500 mL three-neck flask, Intermediate Compound IM-9 (10.00 g, 32.3 mmol), Pd(dba)₂ (0.56 g, 0.03 equiv, 1.0 mmol), NaOtBu (3.10 g, 1.0 equiv, 32.3 mmol), toluene (162 mL), 2-bromo-9-phenyl-9H-carbazole (11.46 g, 1.1 equiv, 35.6 mmol), and PtBu₃ (0.65 g, 0.1 equiv, 3.2 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-10 (12.99 g, yield 73%).

By measuring FAB-MS, a mass number of m/z=550 was observed by molecular ion peak, thereby identifying Intermediate Compound IM-10.

### 3) Synthesis of Compound B36

In an Ar atmosphere, in a 300 mL three-neck flask, Intermediate Compound IM-10 (10.00 g, 18.2 mmol), Pd(dba)₂ (0.31 g, 0.03 equiv, 0.5 mmol), NaOtBu (3.49 g, 2.0 equiv, 36.3 mmol), toluene (91 mL), 4-bromodibenzothiophene (5.26 g, 1.1 equiv, 20.0 mmol), and PtBu₃ (0.37 g, 0.1 equiv, 1.8 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain solid Compound B36 (11.05 g, yield 77%).

By measuring FAB-MS, a mass number of m/z=732 was observed by molecular ion peak, thereby identifying Compound B36.

### (7) Synthesis of Compound B43

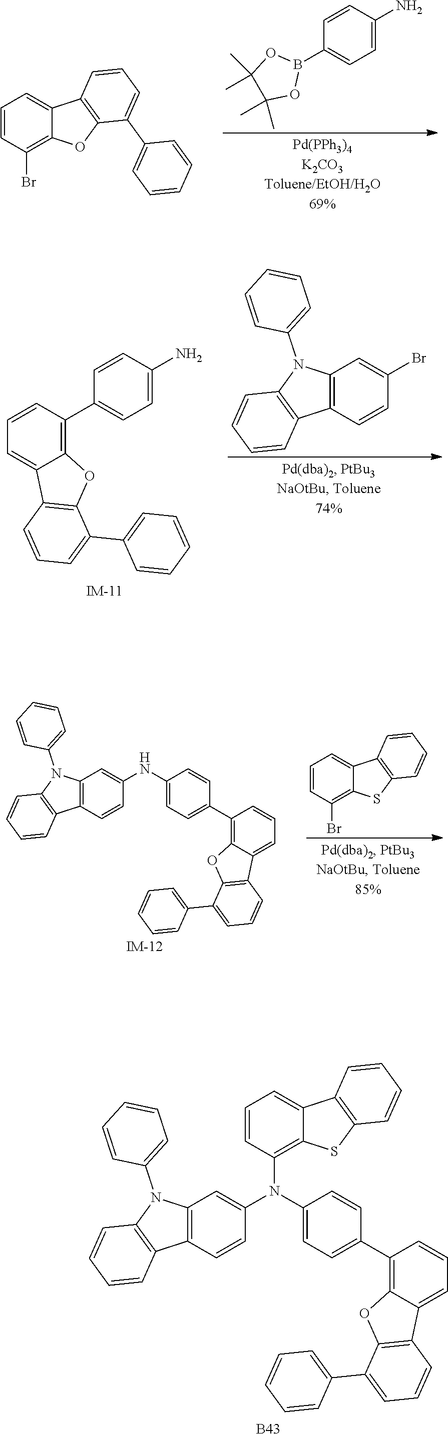

### 1) Synthesis of Intermediate Compound IM-11

In an Ar atmosphere, in a 1,000 mL three-neck flask, 4-bromo-6-phenyldibenzofunran (20.00 g, 61.9 mmol), 4-(4,4,5,5-tetramethyl-1,3,2-dioxaborolan-2-yl)aniline (14.91 g, 1.1 equiv, 68.1 mmol), K₂CO₃ (25.66 g, 3.0 equiv, 185.6 mmol), Pd(PPh₃)₄ (3.58 g, 0.05 eq, 3.1 mmol), and a mixed solution of toluene/EtOH/H₂O (volume ratio=4/2/1) (433 mL) were sequentially added, and heated and stirred at about 80° C. After air-cooled to room temperature, the reaction solution was extracted with toluene. A water layer was removed, and organic layers were washed with saturated saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-11 (14.32 g, yield 69%).

By measuring FAB-MS, a mass number of m/z=335 was observed by molecular ion peak, thereby identifying Intermediate Compound IM-11.

### 2) Synthesis of Intermediate Compound IM-12

In an Ar atmosphere, in a 500 mL three-neck flask, Intermediate Compound IM-11 (10.00 g, 29.8 mmol), Pd(dba)₂ (0.51 g, 0.03 equiv, 0.9 mmol), NaOtBu (2.87 g, 1.0 equiv, 29.8 mmol), toluene (150 mL), 2-bromo-9-phenyl-9H-carbazole (10.57 g, 1.1 equiv, 32.8 mmol), and PtBu₃ (0.60 g, 0.1 equiv, 3.0 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-12 (12.72 g, yield 74%).

By measuring FAB-MS, a mass number of m/z=576 was observed by molecular ion peak, thereby identifying Intermediate Compound IM-12.

### 3) Synthesis of Compound B43

In an Ar atmosphere, in a 300 mL three-neck flask, Intermediate Compound IM-12 (10.00 g, 17.3 mmol), Pd(dba)₂ (0.30 g, 0.03 equiv, 0.5 mmol), NaOtBu (3.33 g, 2.0 equiv, 34.7 mmol), toluene (87 mL), 4-bromodibenzothiophene (5.02 g, 1.1 equiv, 19.1 mmol), and PtBu₃ (0.35 g, 0.1 equiv, 1.7 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain solid Compound B43 (11.58 g, yield 85%).

By measuring FAB-MS, a mass number of m/z=785 was observed by molecular ion peak, thereby identifying Compound B43.

### (8) Synthesis of Compound B86

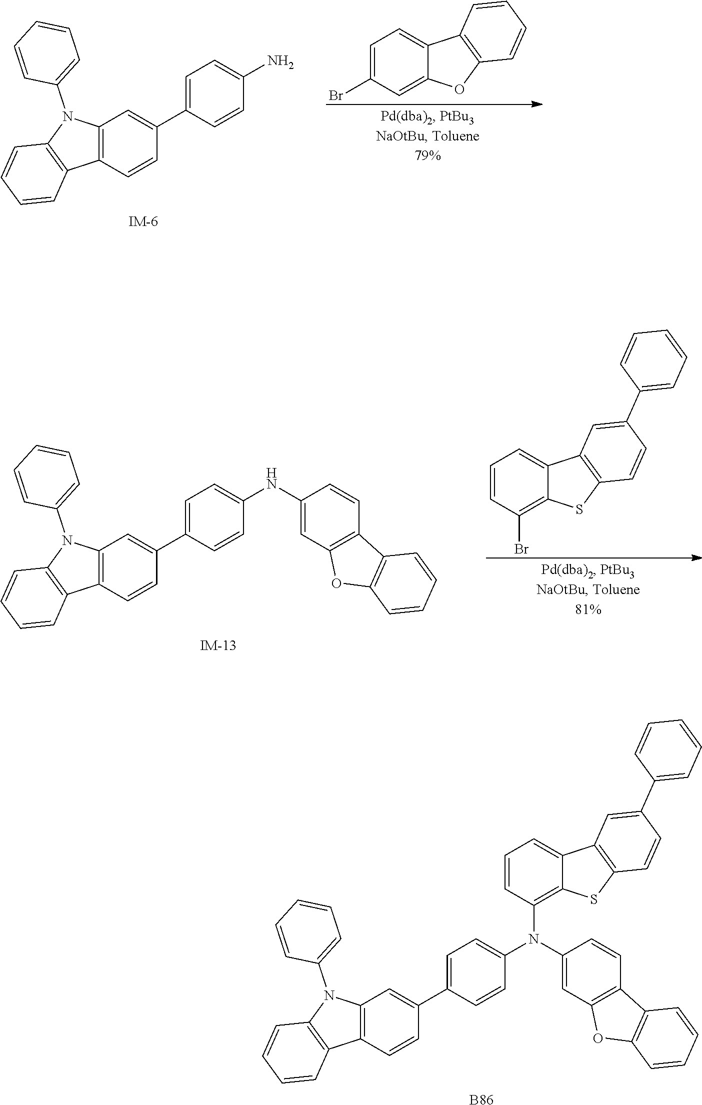

### 1) Synthesis of Intermediate Compound IM-13

In an Ar atmosphere, in a 500 mL three-neck flask, Intermediate Compound IM-6 (10.00 g, 29.9 mmol), Pd(dba)₂ (0.52 g, 0.03 equiv, 0.9 mmol), NaOtBu (2.87 g, 1.0 equiv, 29.9 mmol), toluene (150 mL), 3-bromodibenzofuran (8.13 g, 1.1 equiv, 32.9 mmol), and PtBu₃ (0.60 g, 0.1 equiv, 3.0 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-13 (11.83 g, yield 79%).

By measuring FAB-MS, a mass number of m/z=500 was observed by molecular ion peak, thereby identifying Intermediate Compound IM-13.

### 2) Synthesis of Compound B86

In an Ar atmosphere, in a 300 mL three-neck flask, Intermediate Compound IM-13 (10.00 g, 20.0 mmol), Pd(dba)₂ (0.34 g, 0.03 equiv, 0.6 mmol), NaOtBu (3.84 g, 2.0 equiv, 40.0 mmol), toluene (100 mL), 6-bromo-2-phenyldibenzothiophene (7.45 g, 1.1 equiv, 22.0 mmol), and PtBu₃ (0.40 g, 0.1 equiv, 2.0 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain solid Compound B86 (12.28 g, yield 81%).

By measuring FAB-MS, a mass number of m/z=758 was observed by molecular ion peak, thereby identifying Compound B86.

### (9) Synthesis of Compound C38

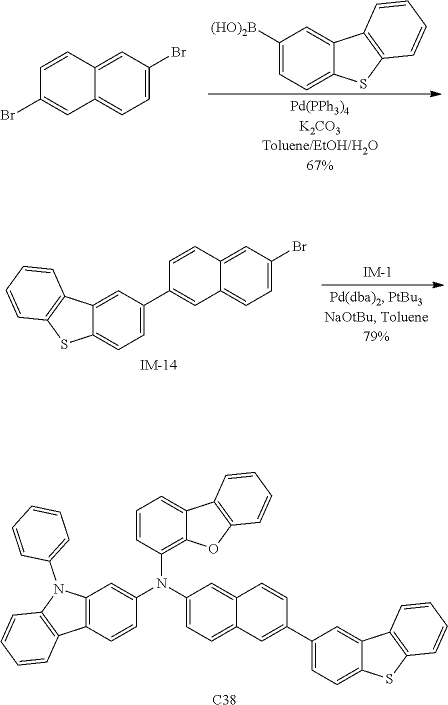

### 1) Synthesis of Intermediate Compound IM-14

In an Ar atmosphere, in a 1,000 mL three-neck flask, 2,6-dibromonaphathalene (20.00 g, 69.9 mmol), dibenzofuran-2-boronic acid (17.55 g, 1.1 equiv, 76.9 mmol), K₂CO₃ (29.0 g, 3.0 equiv, 209.8 mmol), Pd(PPh₃)₄ (4.04 g, 0.05 eq, 3.5 mmol), and a mixed solution of toluene/EtOH/H₂O (volume ratio=4/2/1) (490 mL) were sequentially added, and heated and stirred at about 80° C. After air-cooled to room temperature, the reaction solution was extracted with toluene. A water layer was removed, and organic layers were washed with saturated saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-14 (18.24 g, yield 67%).

By measuring FAB-MS, a mass number of m/z=389 was observed by molecular ion peak, thereby identifying Intermediate Compound IM-14.

### 2) Synthesis of Compound C38

In an Ar atmosphere, in a 300 mL three-neck flask, Intermediate Compound IM-1 (10.00 g, 23.6 mmol), Pd(dba)₂ (0.41 g, 0.03 equiv, 0.7 mmol), NaOtBu (4.53 g, 2.0 equiv, 47.1 mmol), toluene (118 mL), Intermediate Compound IM-14 (10.08 g, 1.1 equiv, 25.9 mmol), and PtBu₃ (0.48 g, 0.1 equiv, 2.4 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain solid Compound C38 (13.64 g, yield 79%).

By measuring FAB-MS, a mass number of m/z=732 was observed by molecular ion peak, thereby identifying Compound C38.

### (10) Synthesis of Compound C61

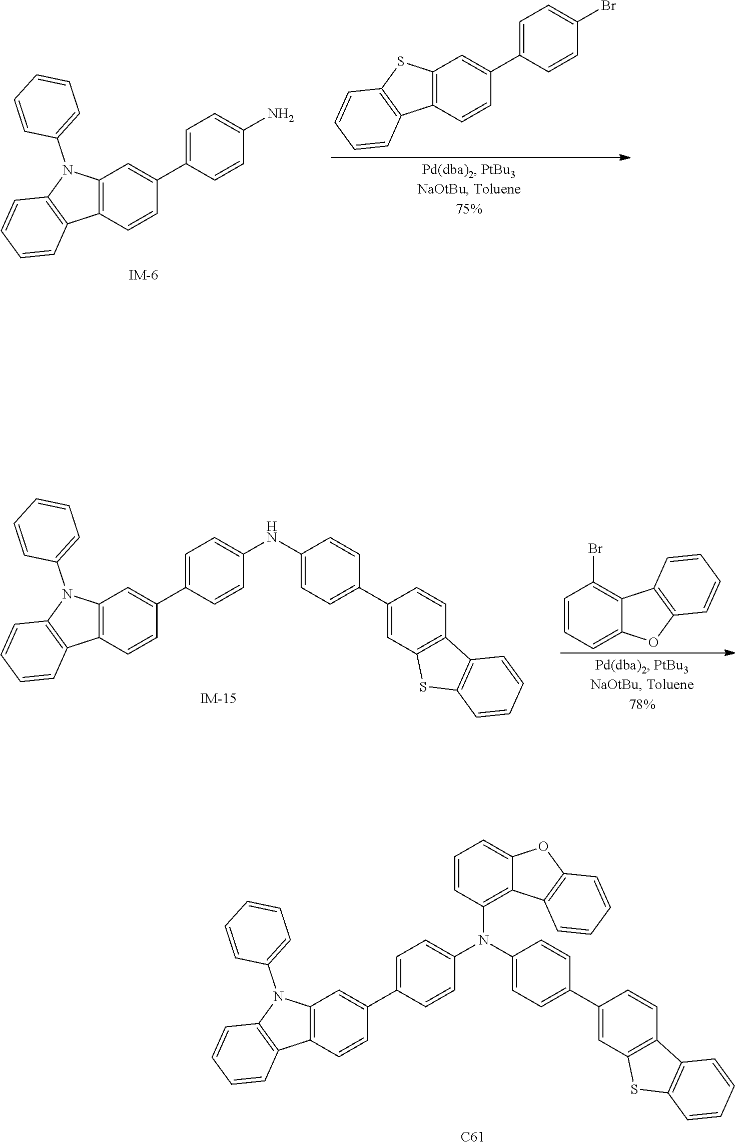

### 1) Synthesis of Intermediate Compound IM-15

In an Ar atmosphere, in a 500 mL three-neck flask, Intermediate Compound IM-6 (10.00 g, 29.9 mmol), Pd(dba)₂ (0.52 g, 0.03 equiv, 0.9 mmol), NaOtBu (2.87 g, 1.0 equiv, 29.9 mmol), toluene (150 mL), 3-(4-bromophenyl)dibenzothiophene (11.16 g, 1.1 equiv, 32.9 mmol), and PtBu₃ (0.60 g, 0.1 equiv, 3.0 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-15 (13.29 g, yield 75%).

By measuring FAB-MS, a mass number of m/z=592 was observed by molecular ion peak, thereby identifying Intermediate Compound IM-15.

### 2) Synthesis of Compound C61

In an Ar atmosphere, in a 300 mL three-neck flask, Intermediate Compound IM-15 (10.00 g, 16.9 mmol), Pd(dba)₂ (0.29 g, 0.03 equiv, 0.5 mmol), NaOtBu (3.24 g, 2.0 equiv, 33.7 mmol), toluene (84 mL), 1-bromodibenzofuran (4.59 g, 1.1 equiv, 18.6 mmol), and PtBu₃ (0.34 g, 0.1 equiv, 1.7 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain solid Compound C61 (9.99 g, yield 78%).

By measuring FAB-MS, a mass number of m/z=758 was observed by molecular ion peak, thereby identifying Compound C61.

### (11) Synthesis of Compound C76

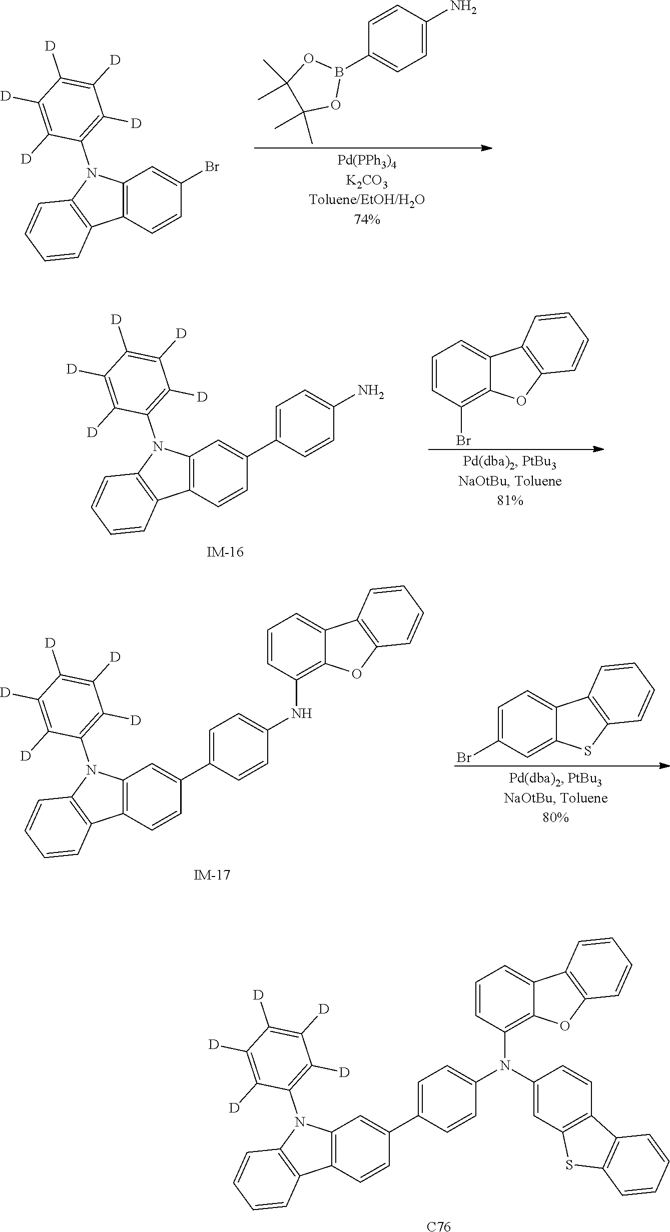

### 1) Synthesis of Intermediate Compound IM-16

In an Ar atmosphere, in a 1,000 mL three-neck flask, 2-bromo-9-(phenyl-d5)-9H-carbazole (20.00 g, 61.1 mmol), 4-(4,4,5,5-tetramethyl-1,3,2-dioxaborolan-2-yl)aniline (14.73 g, 1.1 equiv, 67.2 mmol), K₂CO₃ (25.34 g, 3.0 equiv, 183.4 mmol), Pd(PPh₃)₄ (3.53 g, 0.05 eq, 3.1 mmol), and a mixed solution of toluene/EtOH/H₂O (volume ratio=4/2/1) (428 mL) were sequentially added, and heated and stirred at about 80° C. After air-cooled to room temperature, the reaction solution was extracted with toluene. A water layer was removed, and organic layers were washed with saturated saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-16 (15.35 g, yield 74%).

By measuring FAB-MS, a mass number of m/z=339 was observed by molecular ion peak, thereby identifying Intermediate Compound IM-16.

### 2) Synthesis of Intermediate Compound IM-17

In an Ar atmosphere, in a 500 mL three-neck flask, Intermediate Compound IM-16 (10.00 g, 29.5 mmol), Pd(dba)₂ (0.51 g, 0.03 equiv, 0.9 mmol), NaOtBu (2.83 g, 1.0 equiv, 29.5 mmol), toluene (147 mL), 4-bromodibenzofuran (8.01 g, 1.1 equiv, 32.4 mmol), and PtBu₃ (0.60 g, 0.1 equiv, 3.0 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-17 (12.07 g, yield 81%).

By measuring FAB-MS, a mass number of m/z=505 was observed by molecular ion peak, thereby identifying Intermediate Compound IM-17.

### 3) Synthesis of Compound C76

In an Ar atmosphere, in a 300 mL three-neck flask, Intermediate Compound IM-17 (10.00 g, 19.8 mmol), Pd(dba)₂ (0.34 g, 0.03 equiv, 0.6 mmol), NaOtBu (3.80 g, 2.0 equiv, 39.6 mmol), toluene (99 mL), 3-bromodibenzothiophene (5.72 g, 1.1 equiv, 21.8 mmol), and PtBu₃ (0.40 g, 0.1 equiv, 2.0 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain solid Compound C76 (10.88 g, yield 80%).

By measuring FAB-MS, a mass number of m/z=687 was observed by molecular ion peak, thereby identifying Compound C76.

### (12) Synthesis of Compound C79

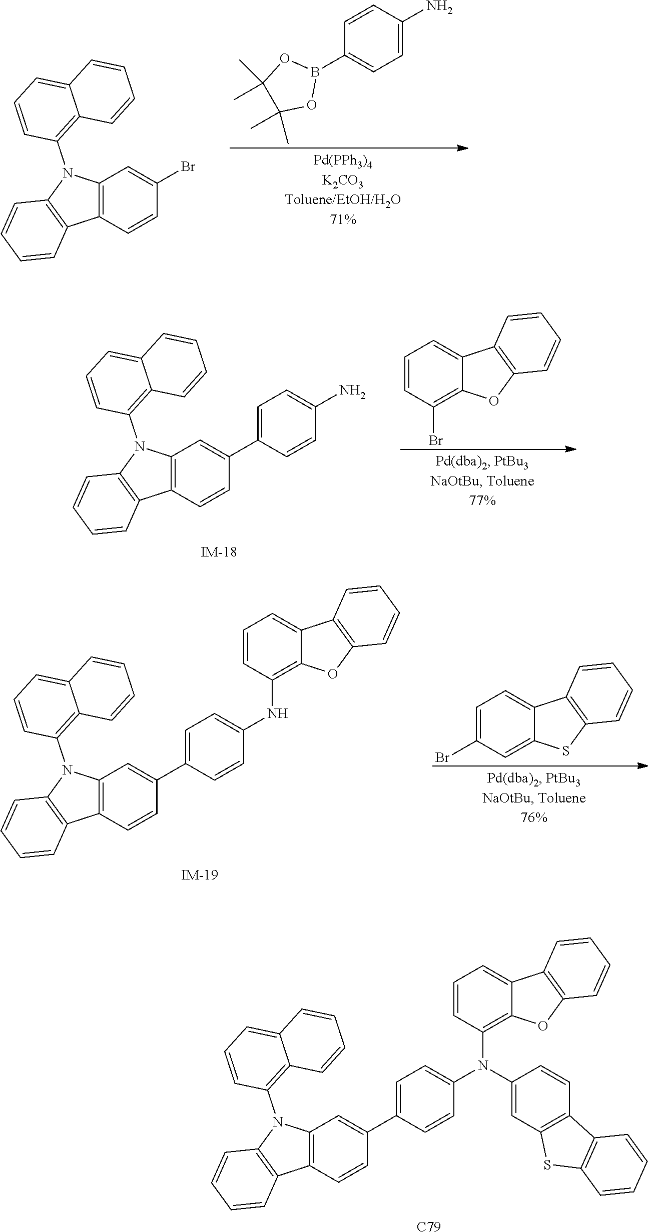

### 1) Synthesis of Intermediate Compound IM-18

In an Ar atmosphere, in a 1,000 mL three-neck flask, 2-bromo-9-(naphthalen-1-yl)-9H-carbazole (20.00 g, 53.7 mmol), 4-(4,4,5,5-tetramethyl-1,3,2-dioxaborolan-2-yl)aniline (12.95 g, 1.1 equiv, 59.1 mmol), K₂CO₃ (22.28 g, 3.0 equiv, 161.2 mmol), Pd(PPh₃)₄ (3.10 g, 0.05 eq, 2.7 mmol), and a mixed solution of toluene/EtOH/H₂O (volume ratio=4/2/1) (376 mL) were sequentially added, and heated and stirred at about 80° C. After air-cooled to room temperature, the reaction solution was extracted with toluene. A water layer was removed, and organic layers were washed with saturated saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-18 (14.67 g, yield 71%).

By measuring FAB-MS, a mass number of m/z=384 was observed by molecular ion peak, thereby identifying Intermediate Compound IM-18.

### 2) Synthesis of Intermediate Compound IM-19

In an Ar atmosphere, in a 500 mL three-neck flask, Intermediate Compound IM-18 (10.00 g, 26.0 mmol), Pd(dba)₂ (0.45 g, 0.03 equiv, 0.8 mmol), NaOtBu (2.45 g, 1.0 equiv, 26.0 mmol), toluene (147 mL), 4-bromodibenzofuran (7.07 g, 1.1 equiv, 28.6 mmol), and PtBu₃ (0.53 g, 0.1 equiv, 2.6 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-19 (11.03 g, yield 77%).

By measuring FAB-MS, a mass number of m/z=550 was observed by molecular ion peak, thereby identifying Intermediate Compound IM-19.

### 3) Synthesis of Compound C79

In an Ar atmosphere, in a 300 mL three-neck flask, Intermediate Compound IM-19 (10.00 g, 18.2 mmol), Pd(dba)₂ (0.31 g, 0.03 equiv, 0.5 mmol), NaOtBu (3.49 g, 2.0 equiv, 36.3 mmol), toluene (91 mL), 3-bromodibenzothiophene (5.26 g, 1.1 equiv, 20.0 mmol), and PtBu₃ (0.37 g, 0.1 equiv, 1.8 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain solid Compound C79 (10.11 g, yield 76%).

By measuring FAB-MS, a mass number of m/z=732 was observed by molecular ion peak, thereby identifying Compound C79.

### (13) Synthesis of Compound D19

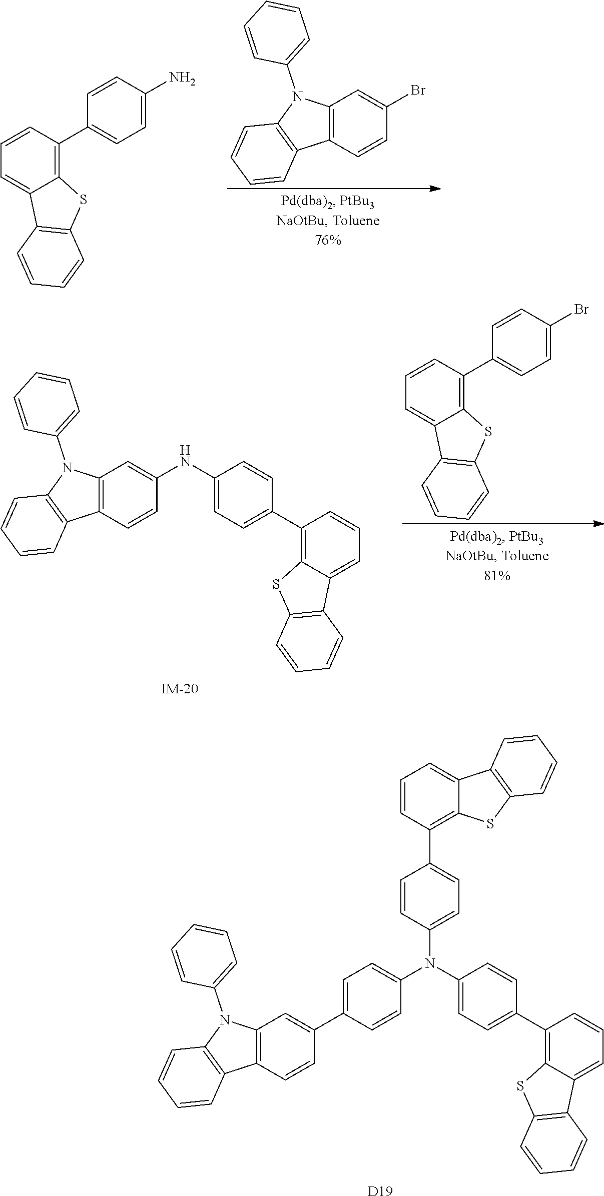

### 1) Synthesis of Intermediate Compound IM-20

In an Ar atmosphere, in a 500 mL three-neck flask, 4-(dibenzothiophen-4-yl)aniline (10.00 g, 36.1 mmol), Pd(dba)₂ (0.63 g, 0.03 equiv, 1.1 mmol), NaOtBu (3.49 g, 1.0 equiv, 36.3 mmol), toluene (181 mL), 2-bromo-9-phenyl-9H-carbazole (12.87 g, 1.1 equiv, 39.9 mmol), and PtBu₃ (0.73 g, 0.1 equiv, 3.6 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-20 (14.26 g, yield 76%).

By measuring FAB-MS, a mass number of m/z=516 was observed by molecular ion peak, thereby identifying Compound IM-20.

### 2) Synthesis of Compound D19

In an Ar atmosphere, in a 300 mL three-neck flask, Intermediate Compound IM-20 (10.00 g, 19.4 mmol), Pd(dba)₂ (0.33 g, 0.03 equiv, 0.6 mmol), NaOtBu (3.72 g, 2.0 equiv, 38.7 mmol), toluene (97 mL), 4-(4-bromophenyl)dibenzothiophene (7.22 g, 1.1 equiv, 21.3 mmol), and PtBu₃ (0.39 g, 0.1 equiv, 1.9 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain solid Compound D19 (12.15 g, yield 81%).

By measuring FAB-MS, a mass number of m/z=775 was observed by molecular ion peak, thereby identifying Compound D19.

### (14) Synthesis of Compound D35

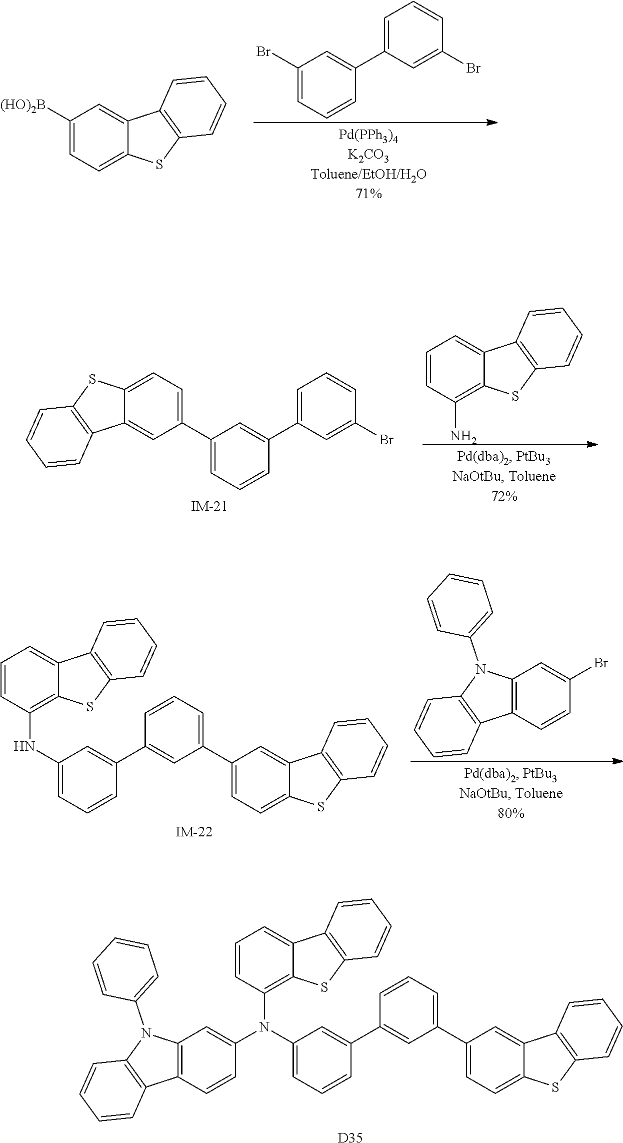

### 1) Synthesis of Intermediate Compound IM-21

In an Ar atmosphere, in a 1,000 mL three-neck flask, dibenzothiophen-2-boronic acid (20.00 g, 87.7 mmol), 3,3′-dibromo-1,1′-biphenyl (30.10 g, 1.1 equiv, 96.5 mmol), K₂CO₃ (36.36 g, 3.0 equiv, 263.1 mmol), Pd(PPh₃)₄ (5.07 g, 0.05 eq, 4.4 mmol), and a mixed solution of toluene/EtOH/H₂O (volume ratio=4/2/1) (613 mL) were sequentially added, and heated and stirred at about 80° C. After air-cooled to room temperature, the reaction solution was extracted with toluene. A water layer was removed, and organic layers were washed with saturated saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-21 (25.86 g, yield 71%).

By measuring FAB-MS, a mass number of m/z=415 was observed by molecular ion peak, thereby identifying Intermediate Compound IM-21.

### 2) Synthesis of Intermediate Compound IM-22

In an Ar atmosphere, in a 500 mL three-neck flask, dibenzothiophene-4-amine (10.00 g, 50.2 mmol), Pd(dba)₂ (0.87 g, 0.03 equiv, 1.5 mmol), NaOtBu (4.82 g, 1.0 equiv, 50.2 mmol), toluene (250 mL), Intermediate Compound IM-21 (22.93 g, 1.1 equiv, 55.2 mmol), and PtBu₃ (1.02 g, 0.1 equiv, 5.0 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-22 (19.28 g, yield 72%).

By measuring FAB-MS, a mass number of m/z=533 was observed by molecular ion peak, thereby identifying Intermediate Compound IM-22.

### 3) Synthesis of Compound D35

In an Ar atmosphere, in a 300 mL three-neck flask, Intermediate Compound IM-22 (10.00 g, 38.6 mmol), Pd(dba)₂ (0.32 g, 0.03 equiv, 0.6 mmol), NaOtBu (3.60 g, 2.0 equiv, 38.6 mmol), toluene (94 mL), 2-bromo-9-phenyl-9H-carbazole (6.64 g, 1.1 equiv, 20.6 mmol), and PtBu₃ (0.38 g, 0.1 equiv, 1.9 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain solid Compound D35 (11.62 g, yield 80%).

By measuring FAB-MS, a mass number of m/z=775 was observed by molecular ion peak, thereby identifying Compound D35.

### (15) Synthesis of Compound D54

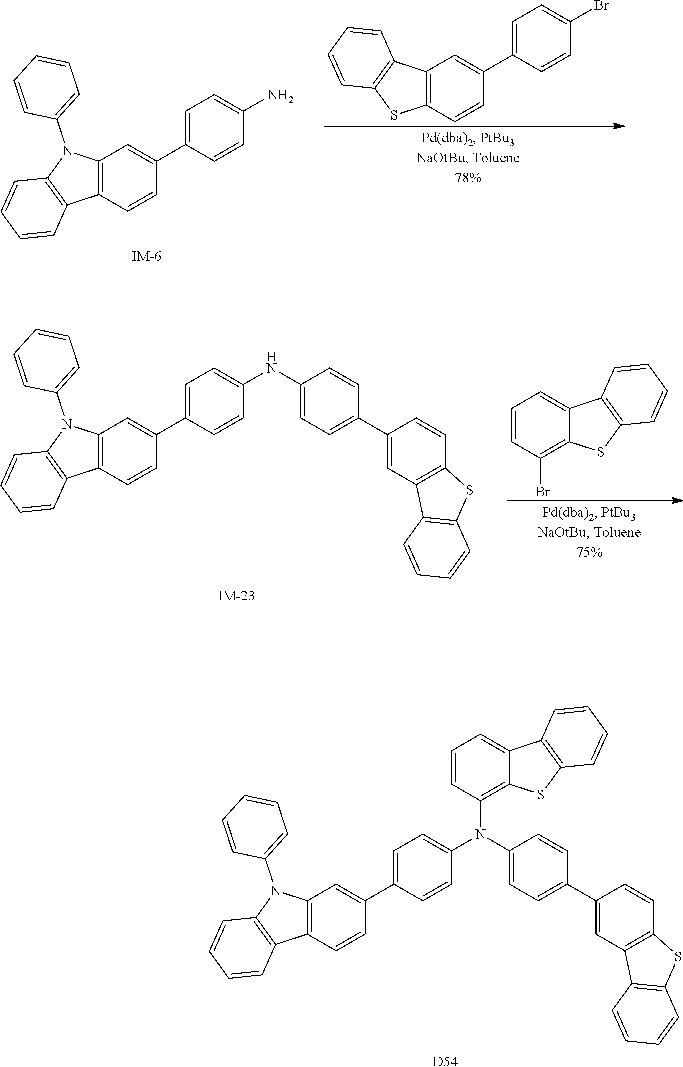

### 1) Synthesis of Intermediate Compound IM-23

In an Ar atmosphere, in a 500 mL three-neck flask, Intermediate Compound IM-6 (10.00 g, 39.9 mmol), Pd(dba)₂ (0.52 g, 0.03 equiv, 0.9 mmol), NaOtBu (2.87 g, 1.0 equiv, 29.9 mmol), toluene (150 mL), 2-(4-bromophenyl)dibenzothiophene (11.16 g, 1.1 equiv, 32.9 mmol), and PtBu₃ (0.60 g, 0.1 equiv, 3.0 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-23 (13.83 g, yield 78%).

By measuring FAB-MS, a mass number of m/z=592 was observed by molecular ion peak, thereby identifying Intermediate Compound IM-23.

### 2) Synthesis of Compound D54

In an Ar atmosphere, in a 300 mL three-neck flask, Intermediate Compound IM-23 (10.00 g, 16.9 mmol), Pd(dba)₂ (0.29 g, 0.03 equiv, 0.5 mmol), NaOtBu (3.24 g, 2.0 equiv, 33.7 mmol), toluene (84 mL), 4-bromodibenzothiophene (4.88 g, 1.1 equiv, 18.6 mmol), and PtBu₃ (0.34 g, 0.1 equiv, 1.7 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain solid Compound D54 (9.81 g, yield 75%).

By measuring FAB-MS, a mass number of m/z=775 was observed by molecular ion peak, thereby identifying Compound D54.

### (16) Synthesis of Compound D81

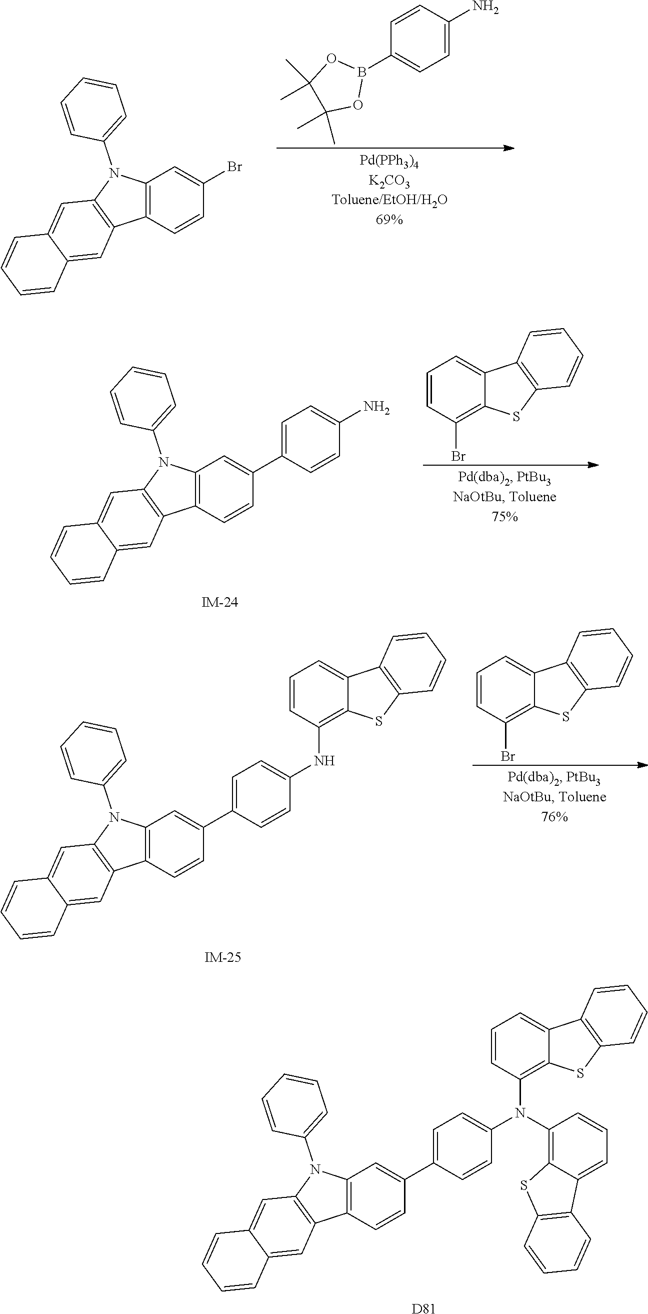

### 1) Synthesis of Intermediate Compound IM-24

In an Ar atmosphere, in a 1,000 mL three-neck flask, 3-bromo-5-phenyl-5H-benzo[b]carbazole (20.00 g, 53.7 mmol), 4-(4,4,5,5-tetramethyl-1,3,2-dioxaborolan-2-yl)aniline (12.95 g, 1.1 equiv, 59.1 mmol), K₂CO₃ (22.28 g, 3.0 equiv, 161.2 mmol), Pd(PPh₃)₄ (3.10 g, 0.05 eq, 2.7 mmol), and a mixed solution of toluene/EtOH/H₂O (volume ratio=4/2/1) (376 mL) were sequentially added, and heated and stirred at about 80° C. After air-cooled to room temperature, the reaction solution was extracted with toluene. A water layer was removed, and organic layers were washed with saturated saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-24 (14.25 g, yield 69%).

By measuring FAB-MS, a mass number of m/z=384 was observed by molecular ion peak, thereby identifying Intermediate Compound IM-24.

### 2) Synthesis of Intermediate Compound IM-25

In an Ar atmosphere, in a 500 mL three-neck flask, Intermediate Compound IM-24 (10.00 g, 26.0 mmol), Pd(dba)₂ (0.45 g, 0.03 equiv, 0.8 mmol), NaOtBu (2.45 g, 1.0 equiv, 26.0 mmol), toluene (147 mL), 4-bromodibenzothiophene (7.53 g, 1.1 equiv, 28.6 mmol), and PtBu₃ (0.53 g, 0.1 equiv, 2.6 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-25 (11.05 g, yield 75%).

By measuring FAB-MS, a mass number of m/z=566 was observed by molecular ion peak, thereby identifying Intermediate Compound IM-25.

### 3) Synthesis of Compound D81

In an Ar atmosphere, in a 300 mL three-neck flask, Intermediate Compound IM-25 (10.00 g, 17.6 mmol), Pd(dba)₂ (0.30 g, 0.03 equiv, 0.5 mmol), NaOtBu (3.39 g, 2.0 equiv, 35.3 mmol), toluene (88 mL), 4-bromodibenzothiophene (5.11 g, 1.1 equiv, 19.4 mmol), and PtBu₃ (0.36 g, 0.1 equiv, 1.8 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain solid Compound D81 (10.04 g, yield 76%).

By measuring FAB-MS, a mass number of m/z=748 was observed by molecular ion peak, thereby identifying Compound D81.

### (17) Synthesis of Compound A39

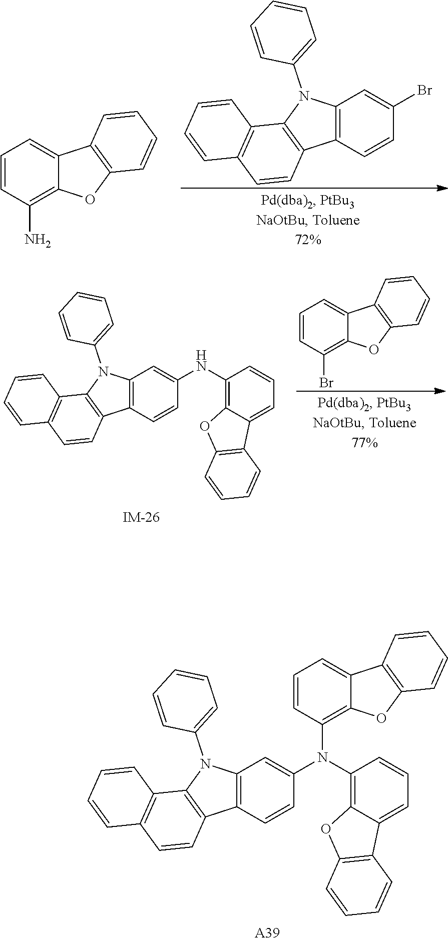

### 1) Synthesis of Intermediate Compound IM-26

In an Ar atmosphere, in a 500 mL three-neck flask, 4-aminodibenzofuran (10.00 g, 54.6 mmol), Pd(dba)₂ (0.94 g, 0.03 equiv, 1.6 mmol), NaOtBu (5.25 g, 1.0 equiv, 54.6 mmol), toluene (272 mL), 9-bromo-11-phenyl-11H-benzo[a]carbazole (22.35 g, 1.1 equiv, 60.0 mmol), and PtBu₃ (1.10 g, 0.1 equiv, 5.5 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-26 (18.65 g, yield 72%).

By measuring FAB-MS, a mass number of m/z=474 was observed by molecular ion peak, thereby identifying Intermediate Compound IM-26.

### 2) Synthesis of Compound A39

In an Ar atmosphere, in a 300 mL three-neck flask, Intermediate Compound IM-26 (10.00 g, 21.1 mmol), Pd(dba)₂ (0.36 g, 0.03 equiv, 0.6 mmol), NaOtBu (4.05 g, 2.0 equiv, 42.1 mmol), toluene (105 mL), 4-bromodibenzofuran (5.73 g, 1.1 equiv, 23.2 mmol), and PtBu₃ (0.43 g, 0.1 equiv, 2.1 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain solid Compound A39 (10.40 g, yield 77%).

By measuring FAB-MS, a mass number of m/z=640 was observed by molecular ion peak, thereby identifying Compound A39.

### (18) Synthesis of Compound B40

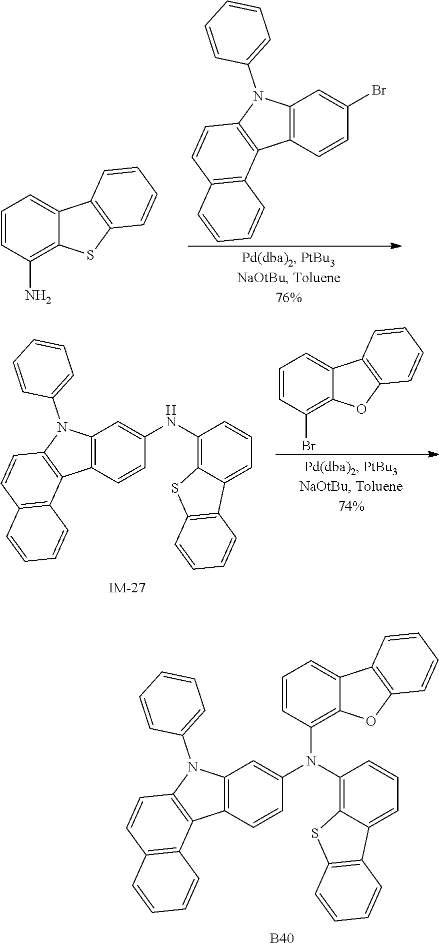

### 1) Synthesis of Intermediate Compound IM-27

In an Ar atmosphere, in a 500 mL three-neck flask, 4-aminodibenzothiophene (10.00 g, 50.2 mmol), Pd(dba)₂ (0.87 g, 0.03 equiv, 1.5 mmol), NaOtBu (4.82 g, 1.0 equiv, 50.2 mmol), toluene (250 mL), 9-bromo-7-phenyl-7H-benzo[c]carbazole (20.55 g, 1.1 equiv, 55.2 mmol), and PtBu₃ (1.02 g, 0.1 equiv, 5.1 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-27 (18.71 g, yield 76%).

By measuring FAB-MS, a mass number of m/z=490 was observed by molecular ion peak, thereby identifying Intermediate Compound IM-27.

### 2) Synthesis of Compound B40

In an Ar atmosphere, in a 300 mL three-neck flask, Intermediate Compound IM-27 (10.00 g, 20.4 mmol), Pd(dba)₂ (0.35 g, 0.03 equiv, 0.6 mmol), NaOtBu (3.92 g, 2.0 equiv, 40.8 mmol), toluene (102 mL), 4-bromodibenzofuran (5.54 g, 1.1 equiv, 22.4 mmol), and PtBu₃ (0.41 g, 0.1 equiv, 2.0 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain solid Compound B40 (9.91 g, yield 74%).

By measuring FAB-MS, a mass number of m/z=656 was observed by molecular ion peak, thereby identifying Compound B40.

### (19) Synthesis of Compound C83

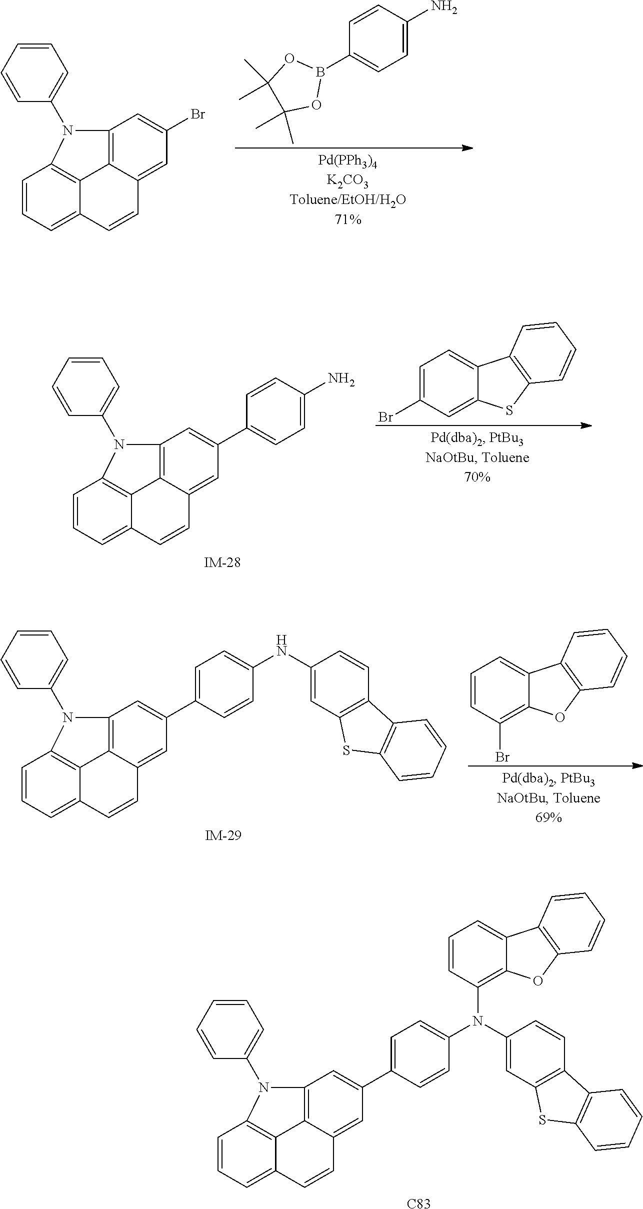

### 1) Synthesis of Intermediate Compound IM-28

In an Ar atmosphere, in a 1,000 mL three-neck flask, 2-bromo-4-phenyl-4H-benzo[def]carbazole (20.00 g, 57.8 mmol), 4-(4,4,5,5-tetramethyl-1,3,2-dioxaborolan-2-yl)aniline (13.92 g, 1.1 equiv, 63.5 mmol), K₂CO₃ (23.95 g, 3.0 equiv, 173.3 mmol), Pd(PPh₃)₄ (3.34 g, 0.05 eq, 2.9 mmol), and a mixed solution of toluene/EtOH/H₂O (volume ratio=4/2/1) (405 mL) were sequentially added, and heated and stirred at about 80° C. After air-cooled to room temperature, the reaction solution was extracted with toluene. A water layer was removed, and organic layers were washed with saturated saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-28 (14.70 g, yield 71%).

By measuring FAB-MS, a mass number of m/z=358 was observed by molecular ion peak, thereby identifying Intermediate Compound IM-28.

### 2) Synthesis of Intermediate Compound IM-29

In an Ar atmosphere, in a 500 mL three-neck flask, Intermediate Compound IM-28 (10.00 g, 27.9 mmol), Pd(dba)₂ (0.48 g, 0.03 equiv, 0.8 mmol), NaOtBu (2.68 g, 1.0 equiv, 27.9 mmol), toluene (140 mL), 3-bromodibenzothiophene (8.08 g, 1.1 equiv, 30.7 mmol), and PtBu₃ (0.56 g, 0.1 equiv, 2.8 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain Intermediate Compound IM-29 (10.56 g, yield 70%).

By measuring FAB-MS, a mass number of m/z=540 was observed by molecular ion peak, thereby identifying Intermediate Compound IM-29.

### 3) Synthesis of Compound C83

In an Ar atmosphere, in a 300 mL three-neck flask, Intermediate Compound IM-29 (10.00 g, 18.5 mmol), Pd(dba)₂ (0.32 g, 0.03 equiv, 0.6 mmol), NaOtBu (3.55 g, 2.0 equiv, 37.0 mmol), toluene (93 mL), 4-bromodibenzofuran (5.03 g, 1.1 equiv, 20.3 mmol), and PtBu₃ (0.37 g, 0.1 equiv, 1.8 mmol) were sequentially added, and then heated and stirred under reflux. After the reaction solution was air-cooled to room temperature, organic layers were separated and obtained by adding water to the reaction solvent. The organic layers were further extracted by adding toluene to a water layer, and then the organic layers were combined and washed with saline and then dried over MgSO₄. MgSO₄ was filtered off and the organic layers were concentrated, and then the resulting crude product was purified by silica gel column chromatography (utilizing a mixed solvent of hexane and toluene as an eluent) to obtain solid Compound C83 (9.02 g, yield 69%).

By measuring FAB-MS, a mass number of m/z=706 was observed by molecular ion peak, thereby identifying Compound C83.

### 2. Manufacture and Evaluation of Light Emitting Element

Evaluation of the light emitting elements including compounds of Examples and Comparative Examples in a hole transport region was performed as follows. The method for manufacturing the light emitting element for the evaluation of the element is described below.

### (1) Manufacture of Light Emitting Element 1

A 1500 Å-thick ITO was patterned on a glass substrate, and then the glass substrate was washed with ultrapure water and treated with UV and ozone for about 10 minutes to form a first electrode. Thereafter, 2-TNATA was deposited to form a 600 Å-thick hole injection layer. Then, Example Compound or Comparative Example Compound was deposited to form a 300 Å-thick hole transport layer.

Thereafter, TBP was doped to ADN by 3% to form a 250 Å-thick emission layer. Then, Alq₃ was deposited to form a 250 Å-thick electron transport layer, and LiF was deposited to form a 10 Å-thick electron injection layer.

Then, aluminum (Al) was provided to form a 1,000 Å-thick second electrode.

In the Examples, the hole injection layer, the hole transport layer, the emission layer, the electron transport layer, the electron injection layer, and the second electrode were formed by utilizing a vacuum deposition apparatus.

### (2) Manufacture of Light Emitting Element 2

A 1500 Å-thick ITO was patterned on a glass substrate, and then the glass substrate was washed with ultrapure water and treated with UV and ozone for about 10 minutes to form a first electrode. Thereafter, 2-TNATA was deposited to form a 600 Å-thick hole injection layer. Then, H-1-1 was deposited to form a 200 Å-thick hole transport layer, and then Example Compound or Comparative Example Compound was deposited to form 100 Å-thick electron blocking layer.

Thereafter, TBP was doped to ADN by 3% to form a 250 Å-thick emission layer. Then, Alq₃ was deposited to form a 250 Å-thick electron transport layer, and LiF was deposited to form a 10 Å-thick electron injection layer.

Then, aluminum (Al) was provided to form a 1,000 Å-thick second electrode.

In the Examples, the hole injection layer, the hole transport layer, the electron blocking layer, the emission layer, the electron transport layer, the electron injection layer, and the second electrode were formed by utilizing a vacuum deposition apparatus.

In some embodiments, for the molecular weight of Example Compound, FAB-MS was measured by utilizing JMS-700V manufactured by JEOL, Ltd. In some embodiments, for the NMR of Example Compound, ¹H-NMR was measured by utilizing AVAVCE300M manufactured by Bruker Biospin K.K. In the following evaluation of the light emitting elements, current densities, voltages and luminous efficiencies of the elements were measured in a dark room by utilizing 2400 Series Source Meter manufactured by Keithley Instruments, Inc., CS-200, Color and Luminance Meter manufactured by Konica Minolta, Inc., and PC Program LaVIEW 8.2 for the measurement manufactured by Japan National Instrument, Inc.

Example Compounds and Comparative Example Compounds utilized to manufacture light emitting element 1 and light emitting element 2 are as follows:

### Example Compounds

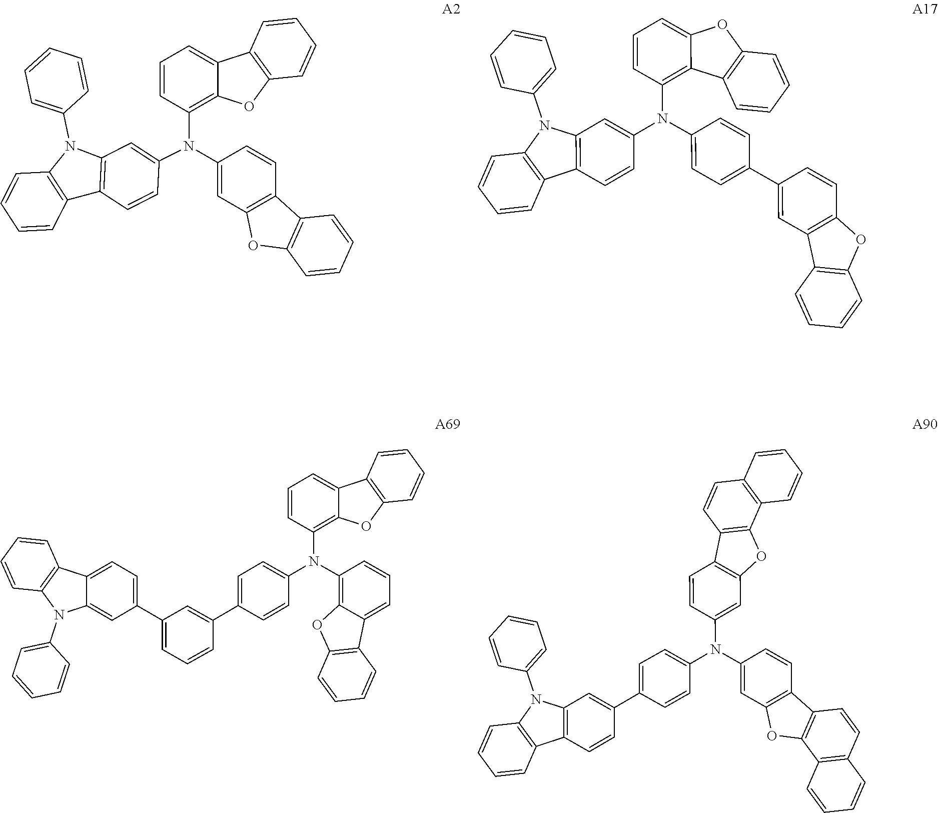 [SMILES: c1ccc(-n2c3ccccc3c3ccc(N(c4ccc5c(c4)oc4ccccc45)c4cccc5c4oc4ccccc45)cc32)cc1 ; c1ccc(-n2c3ccccc3c3ccc(N(c4ccc(-c5ccc6oc7ccccc7c6c5)cc4)c4cccc5oc6ccccc6c45)cc32)cc1 ; c1ccc(-n2c3ccccc3c3ccc(-c4cccc(-c5ccc(N(c6cccc7c6oc6ccccc67)c6cccc7c6oc6ccccc67)cc5)c4)cc32)cc1 ; c1ccc(-n2c3ccccc3c3ccc(-c4ccc(N(c5ccc6c(c5)oc5c7ccccc7ccc65)c5ccc6c(c5)oc5c7ccccc7ccc65)cc4)cc32)cc1]

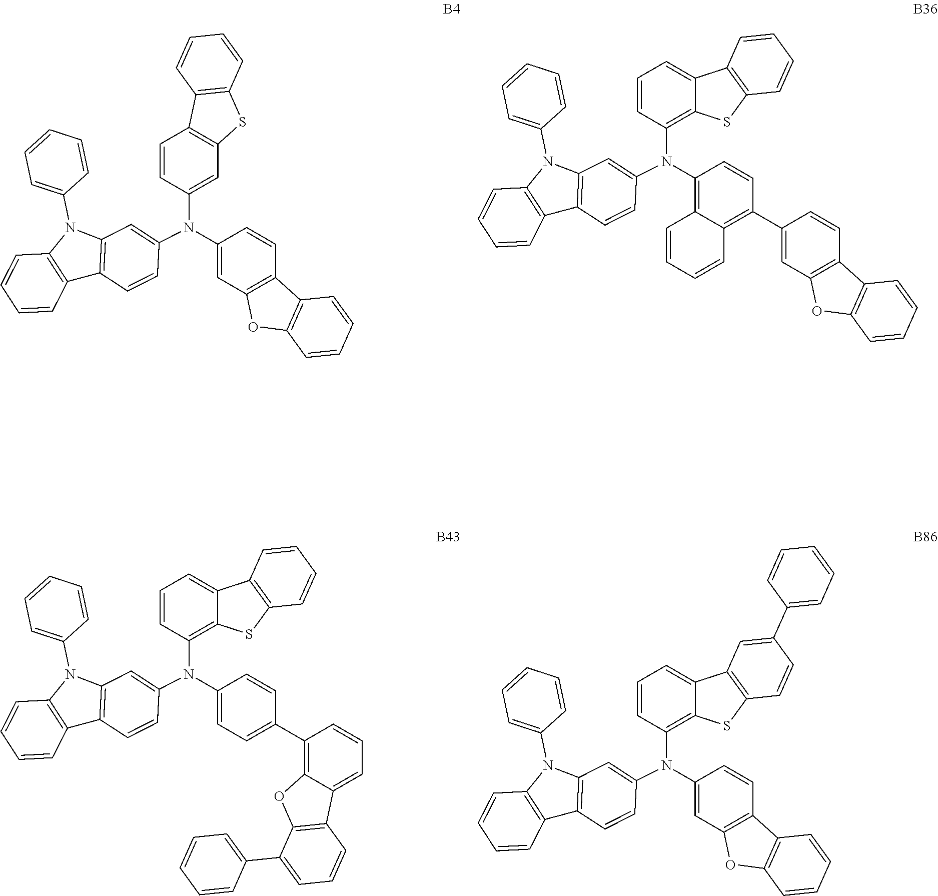 [SMILES: c1ccc(-n2c3ccccc3c3ccc(N(c4ccc5c(c4)oc4ccccc45)c4ccc5c(c4)sc4ccccc45)cc32)cc1 ; c1ccc(-n2c3ccccc3c3ccc(N(c4ccc(-c5ccc6c(c5)oc5ccccc56)c5ccccc45)c4cccc5c4sc4ccccc45)cc32)cc1 ; c1ccc(-c2cccc3c2oc2c(-c4ccc(N(c5ccc6c7ccccc7n(-c7ccccc7)c6c5)c5cccc6c5sc5ccccc56)cc4)cccc23)cc1 ; c1ccc(-c2ccc3sc4c(N(c5ccc6c(c5)oc5ccccc56)c5ccc6c7ccccc7n(-c7ccccc7)c6c5)cccc4c3c2)cc1]

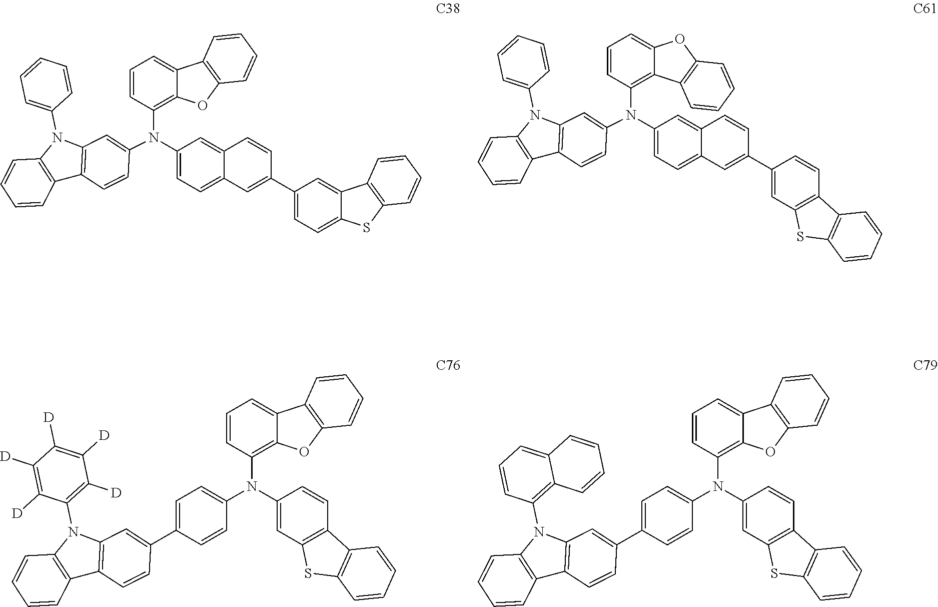 [SMILES: c1ccc(-n2c3ccccc3c3ccc(N(c4ccc5cc(-c6ccc7sc8ccccc8c7c6)ccc5c4)c4cccc5c4oc4ccccc45)cc32)cc1 ; c1ccc(-n2c3ccccc3c3ccc(N(c4ccc5cc(-c6ccc7c(c6)sc6ccccc67)ccc5c4)c4cccc5oc6ccccc6c45)cc32)cc1 ; [2H]c1c([2H])c([2H])c(-n2c3ccccc3c3ccc(-c4ccc(N(c5ccc6c(c5)sc5ccccc56)c5cccc6c5oc5ccccc56)cc4)cc32)c([2H])c1[2H] ; c1ccc2c(-n3c4ccccc4c4ccc(-c5ccc(N(c6ccc7c(c6)sc6ccccc67)c6cccc7c6oc6ccccc67)cc5)cc43)cccc2c1]

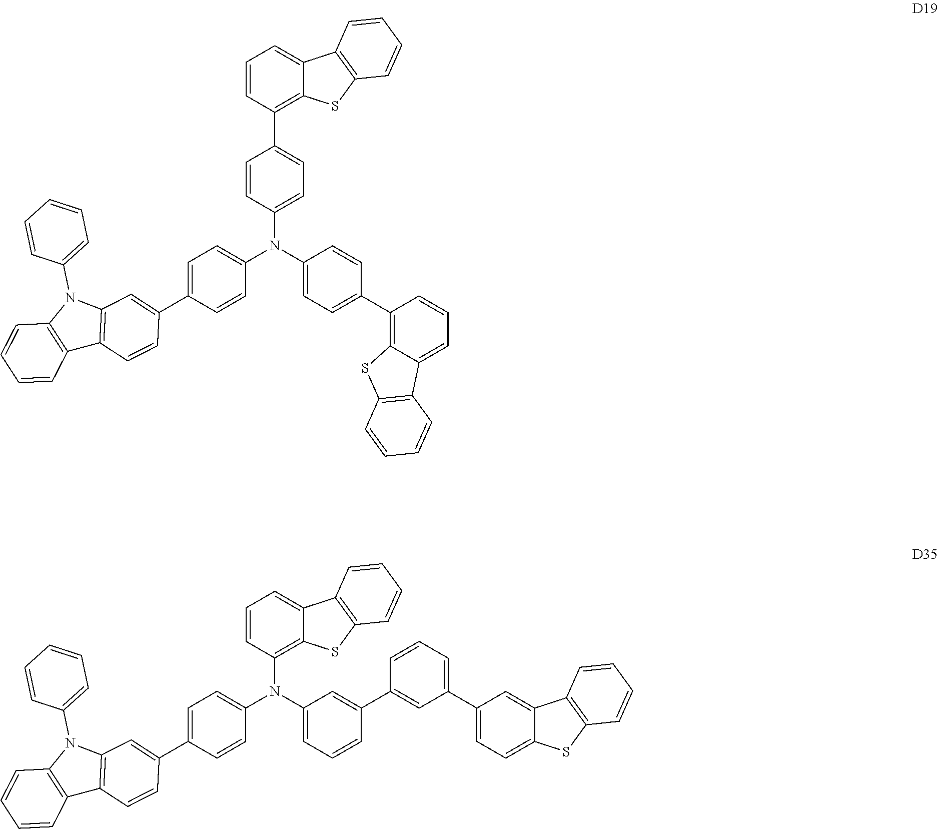 [SMILES: c1ccc(-n2c3ccccc3c3ccc(-c4ccc(N(c5ccc(-c6cccc7c6sc6ccccc67)cc5)c5ccc(-c6cccc7c6sc6ccccc67)cc5)cc4)cc32)cc1 ; c1ccc(-n2c3ccccc3c3ccc(-c4ccc(N(c5cccc(-c6cccc(-c7ccc8sc9ccccc9c8c7)c6)c5)c5cccc6c5sc5ccccc56)cc4)cc32)cc1]

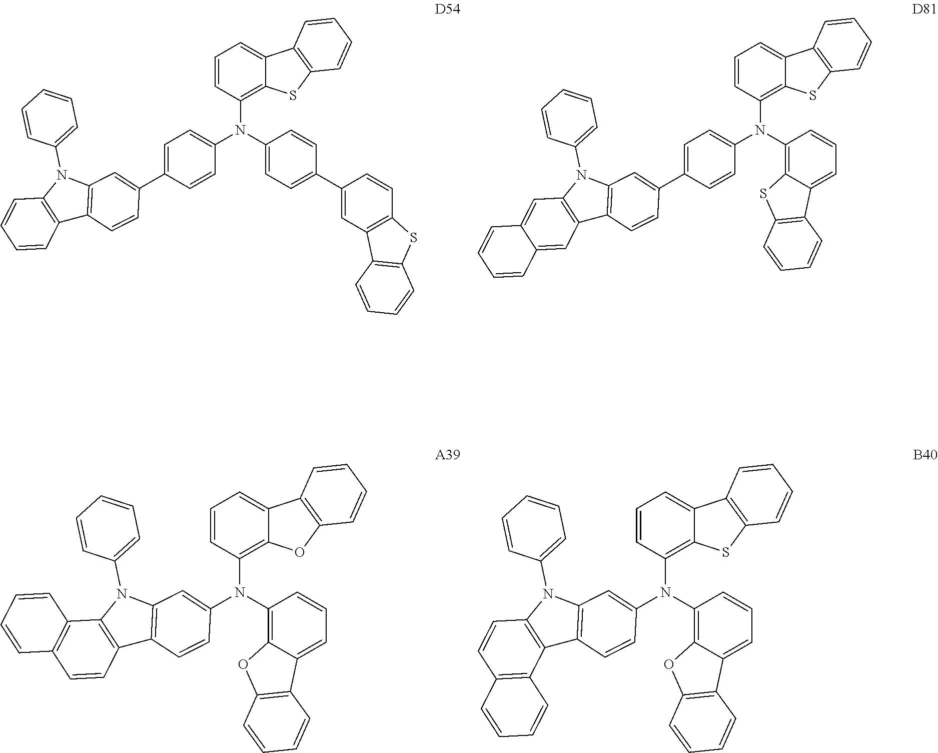 [SMILES: c1ccc(-n2c3ccccc3c3ccc(-c4ccc(N(c5ccc(-c6ccc7sc8ccccc8c7c6)cc5)c5cccc6c5sc5ccccc56)cc4)cc32)cc1 ; c1ccc(-n2c3cc(-c4ccc(N(c5cccc6c5sc5ccccc56)c5cccc6c5sc5ccccc56)cc4)ccc3c3cc4ccccc4cc32)cc1 ; c1ccc(-n2c3cc(N(c4cccc5c4oc4ccccc45)c4cccc5c4oc4ccccc45)ccc3c3ccc4ccccc4c32)cc1 ; c1ccc(-n2c3cc(N(c4cccc5c4oc4ccccc45)c4cccc5c4sc4ccccc45)ccc3c3c4ccccc4ccc32)cc1]

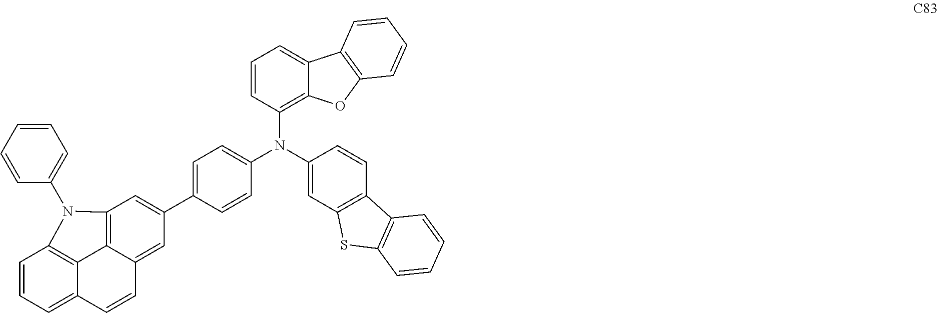 [SMILES: c1ccc(-n2c3cccc4ccc5cc(-c6ccc(N(c7ccc8c(c7)sc7ccccc78)c7cccc8c7oc7ccccc78)cc6)cc2c5c43)cc1]

### Comparative Example Compounds

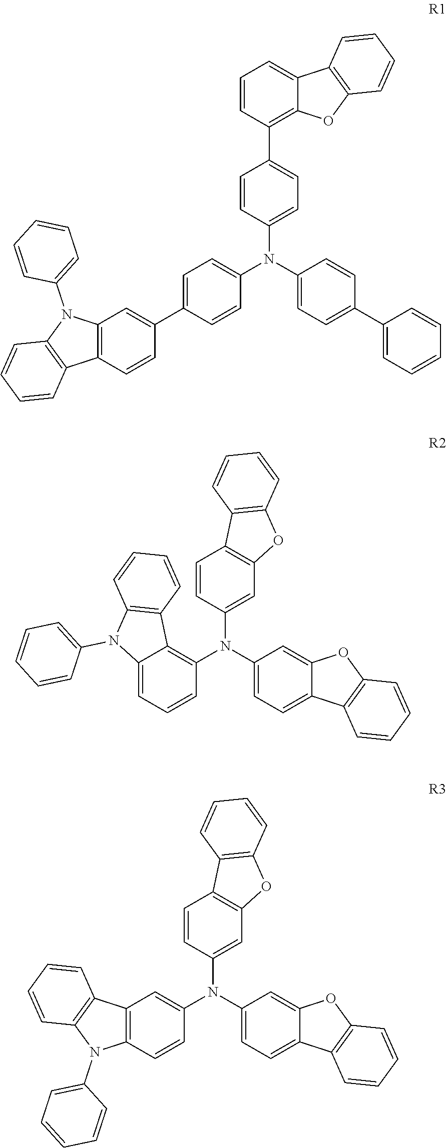 [SMILES: c1ccc(-c2ccc(N(c3ccc(-c4ccc5c6ccccc6n(-c6ccccc6)c5c4)cc3)c3ccc(-c4cccc5c4oc4ccccc45)cc3)cc2)cc1 ; c1ccc(-n2c3ccccc3c3c(N(c4ccc5c(c4)oc4ccccc45)c4ccc5c(c4)oc4ccccc45)cccc32)cc1 ; c1ccc(-n2c3ccccc3c3cc(N(c4ccc5c(c4)oc4ccccc45)c4ccc5c(c4)oc4ccccc45)ccc32)cc1]

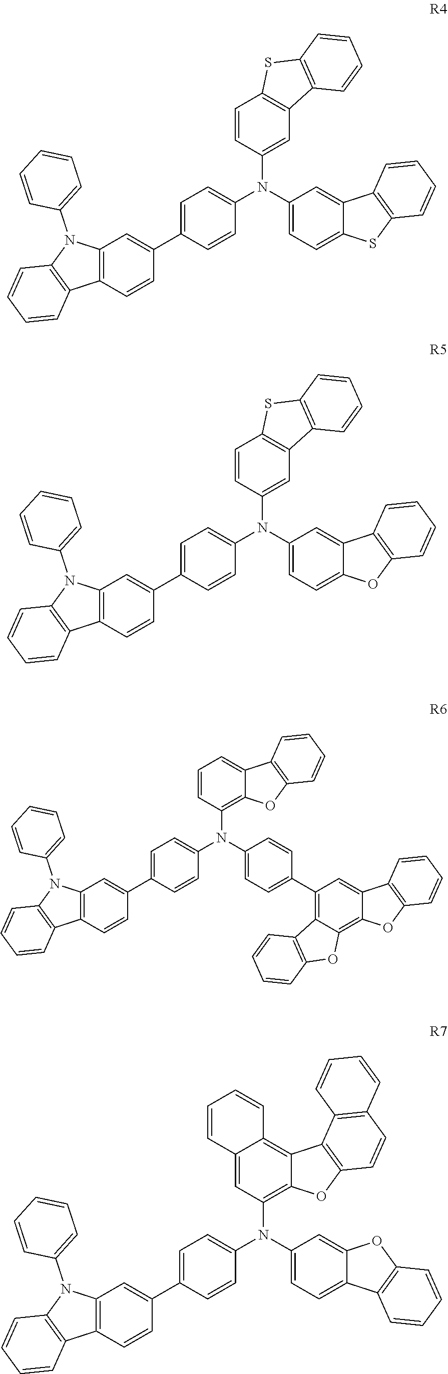 [SMILES: c1ccc(-n2c3ccccc3c3ccc(-c4ccc(N(c5ccc6sc7ccccc7c6c5)c5ccc6sc7ccccc7c6c5)cc4)cc32)cc1 ; c1ccc(-n2c3ccccc3c3ccc(-c4ccc(N(c5ccc6oc7ccccc7c6c5)c5ccc6sc7ccccc7c6c5)cc4)cc32)cc1 ; c1ccc(-n2c3ccccc3c3ccc(-c4ccc(N(c5ccc(-c6cc7c8ccccc8oc7c7oc8ccccc8c67)cc5)c5cccc6c5oc5ccccc56)cc4)cc32)cc1 ; c1ccc(-n2c3ccccc3c3ccc(-c4ccc(N(c5ccc6c(c5)oc5ccccc56)c5cc6ccccc6c6c5oc5ccc7ccccc7c56)cc4)cc32)cc1]

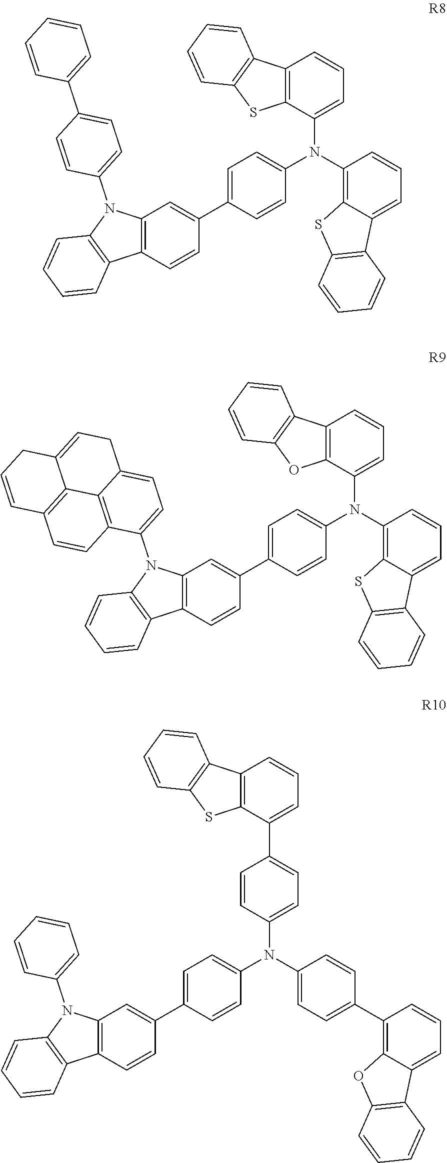 [SMILES: c1ccc(-c2ccc(-n3c4ccccc4c4ccc(-c5ccc(N(c6cccc7c6sc6ccccc67)c6cccc7c6sc6ccccc67)cc5)cc43)cc2)cc1 ; C1=Cc2ccc3c(-n4c5ccccc5c5ccc(-c6ccc(N(c7cccc8c7oc7ccccc78)c7cccc8c7sc7ccccc78)cc6)cc54)ccc4c3c2C(=CC4)C1 ; c1ccc(-n2c3ccccc3c3ccc(-c4ccc(N(c5ccc(-c6cccc7c6oc6ccccc67)cc5)c5ccc(-c6cccc7c6sc6ccccc67)cc5)cc4)cc32)cc1]

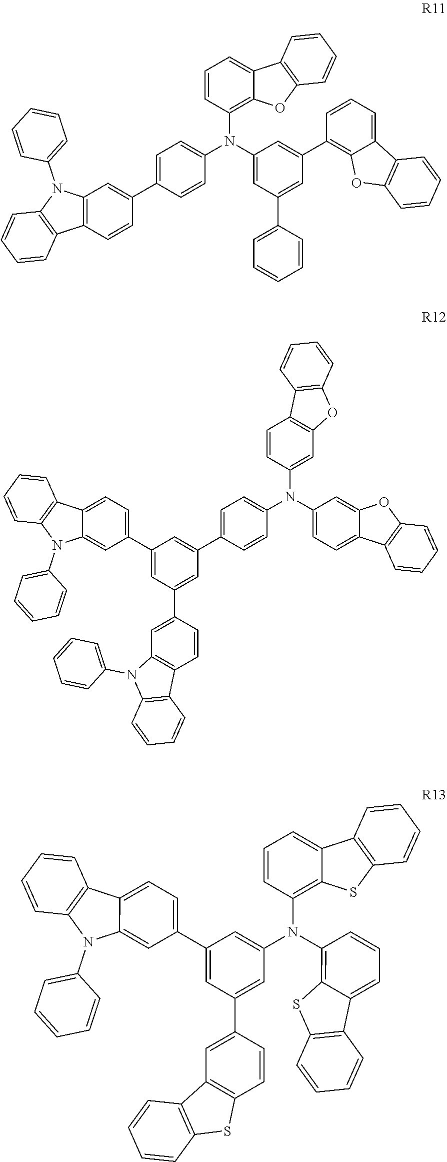 [SMILES: c1ccc(-c2cc(-c3cccc4c3oc3ccccc34)cc(N(c3ccc(-c4ccc5c6ccccc6n(-c6ccccc6)c5c4)cc3)c3cccc4c3oc3ccccc34)c2)cc1 ; c1ccc(-n2c3ccccc3c3ccc(-c4cc(-c5ccc(N(c6ccc7c(c6)oc6ccccc67)c6ccc7c(c6)oc6ccccc67)cc5)cc(-c5ccc6c7ccccc7n(-c7ccccc7)c6c5)c4)cc32)cc1 ; c1ccc(-n2c3ccccc3c3ccc(-c4cc(-c5ccc6sc7ccccc7c6c5)cc(N(c5cccc6c5sc5ccccc56)c5cccc6c5sc5ccccc56)c4)cc32)cc1]

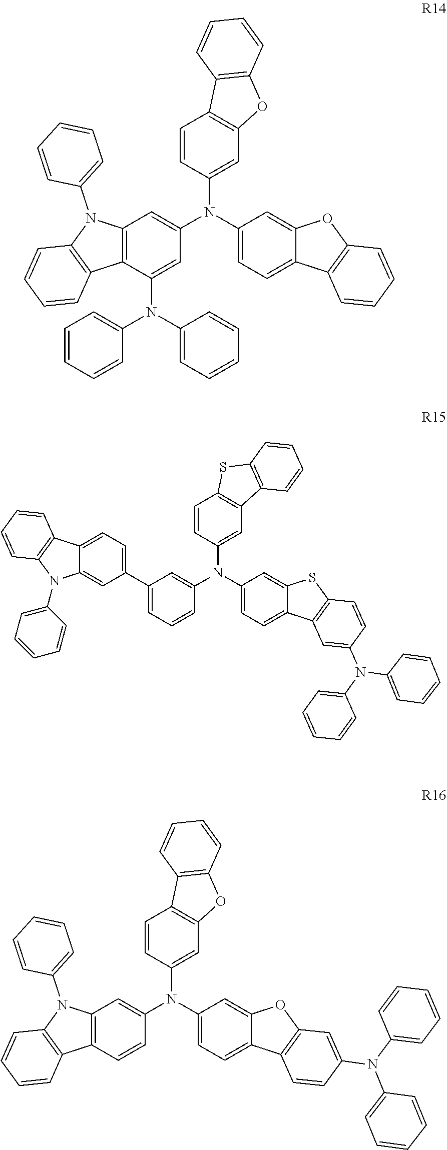 [SMILES: c1ccc(N(c2ccccc2)c2cc(N(c3ccc4c(c3)oc3ccccc34)c3ccc4c(c3)oc3ccccc34)cc3c2c2ccccc2n3-c2ccccc2)cc1 ; c1ccc(N(c2ccccc2)c2ccc3sc4cc(N(c5cccc(-c6ccc7c8ccccc8n(-c8ccccc8)c7c6)c5)c5ccc6sc7ccccc7c6c5)ccc4c3c2)cc1 ; c1ccc(N(c2ccccc2)c2ccc3c(c2)oc2cc(N(c4ccc5c(c4)oc4ccccc45)c4ccc5c6ccccc6n(-c6ccccc6)c5c4)ccc23)cc1]

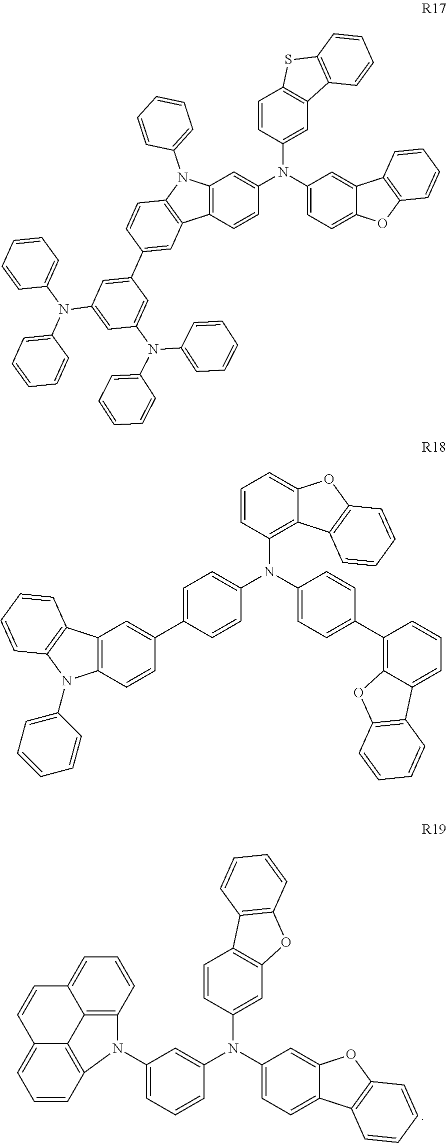 [SMILES: c1ccc(N(c2ccccc2)c2cc(-c3ccc4c(c3)c3ccc(N(c5ccc6oc7ccccc7c6c5)c5ccc6sc7ccccc7c6c5)cc3n4-c3ccccc3)cc(N(c3ccccc3)c3ccccc3)c2)cc1 ; c1ccc(-n2c3ccccc3c3cc(-c4ccc(N(c5ccc(-c6cccc7c6oc6ccccc67)cc5)c5cccc6oc7ccccc7c56)cc4)ccc32)cc1 ; c1cc(N(c2ccc3c(c2)oc2ccccc23)c2ccc3c(c2)oc2ccccc23)cc(-n2c3cccc4ccc5cccc2c5c43)c1]

In some embodiments, compounds of each functional layer utilized to manufacture light emitting elements 1 and 2 are as follows:

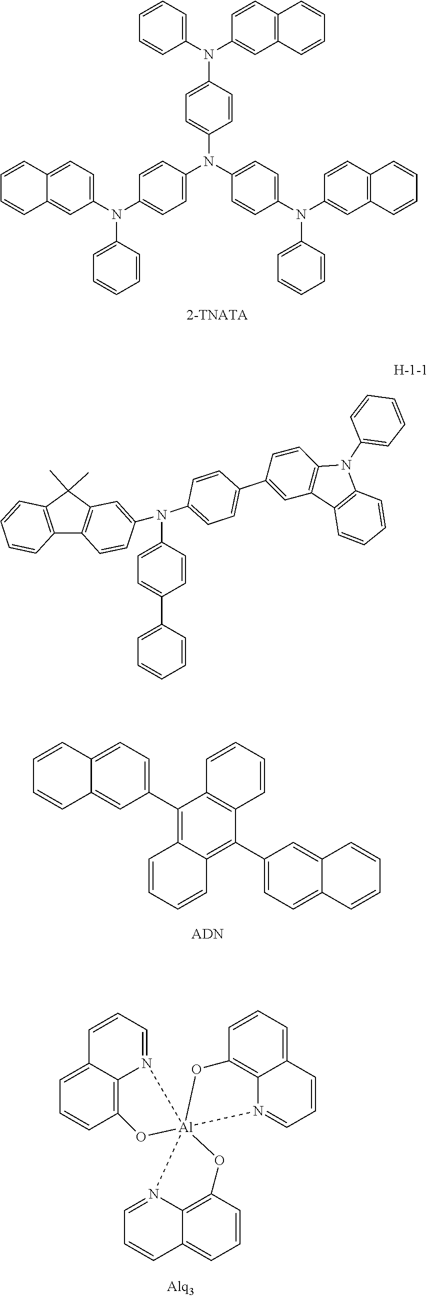

### (3) Evaluation of Light Emitting Element 1 and Light Emitting Element 2

The evaluation results of light emitting element 1 with respect to Examples 1-1 to 1-19 and Comparative Examples 1-1 to 1-19 are listed in Table 1, and the evaluation results of light emitting element 2 with respect to Examples 2-1 to 2-19 and Comparative Examples 2-1 to 2-19 are listed in Table 2. The maximum luminous efficiencies and half service lives of light emitting element 1 and light emitting element 2 are listed in comparison in each of Tables 1 and 2. In the evaluation results of the characteristics for Examples and Comparative Examples listed in Tables 1 and 2, the luminous efficiency shows the efficiency value at a current density of 10 mA/cm².

The element service life is a relative value showing a time when the brightness value is 50% of an initial brightness during the substantially continuous operation of the element at 1.0 mA/Cm² compared with Comparative Examples 1-1 and 2-1.

The luminous efficiencies and element service lifes in Tables 1 and 2 represent compared values when it is assumed that each of the luminous efficiency and service life of Comparative Examples 1-1 and 2-1, respectively, is 100%.

TABLE 1

| Examples of | | Luminous | Element |
|---|---|---|---|
| elements | | efficiency | service life |
| manufactured | Hole transport layer | @10 mA/cm² | LT50 |
| Example 1-1 | Example Compound | 145% | 155% |
| | A2 | | |
| Example 1-2 | Example Compound | 147% | 149% |
| | A17 | | |
| Example 1-3 | Example Compound | 155% | 148% |
| | A69 | | |
| Example 1-4 | Example Compound | 138% | 163% |
| | A90 | | |
| Example 1-5 | Example Compound | 140% | 160% |
| | B4 | | |
| Example 1-6 | Example Compound | 146% | 147% |
| | B36 | | |
| Example 1-7 | Example Compound | 145% | 150% |
| | B43 | | |
| Example 1-8 | Example Compound | 139% | 152% |
| | B86 | | |
| Example 1-9 | Example Compound | 138% | 155% |
| | C38 | | |
| Example 1- | Example Compound | 137% | 157% |
| 10 | C61 | | |
| Example 1- | Example Compound | 139% | 155% |
| 11 | C76 | | |
| Example 1- | Example Compound | 137% | 154% |
| 12 | C79 | | |
| Example 1- | Example Compound | 142% | 152% |
| 13 | D19 | | |
| Example 1- | Example Compound | 153% | 148% |
| 14 | D35 | | |
| Example 1- | Example Compound | 147% | 156% |
| 15 | D54 | | |
| Example 1- | Example Compound | 149% | 159% |
| 16 | D81 | | |
| Example 1- | Example Compound | 147% | 162% |
| 17 | A39 | | |
| Example 1- | Example Compound | 144% | 155% |
| 18 | B40 | | |
| Example 1- | Example Compound | 150% | 150% |
| 19 | C83 | | |
| Comparative | Comparative | 100% | 100% |
| Example 1-1 | Example Compound | | |
| | R1 | | |
| Comparative | Comparative | 105% | 75% |
| Example 1-2 | Example Compound | | |
| | R2 | | |
| Comparative | Comparative | 102% | 87% |
| Example 1-3 | Example Compound | | |
| | R3 | | |
| Comparative | Comparative | 107% | 110% |
| Example 1-4 | Example Compound | | |
| | R4 | | |
| Comparative | Comparative | 108% | 105% |
| Example 1-5 | Example Compound | | |
| | R5 | | |
| Comparative | Comparative | 94% | 76% |
| Example 1-6 | Example Compound | | |
| | R6 | | |
| Comparative | Comparative | 99% | 80% |
| Example 1-7 | Example Compound | | |
| | R7 | | |
| Comparative | Comparative | 110% | 98% |
| Example 1-8 | Example Compound | | |
| | R8 | | |
| Comparative | Comparative | 85% | 64% |
| Example 1-9 | Example Compound | | |
| | R9 | | |
| Comparative | Comparative | 115% | 60% |
| Example 1- | Example Compound | | |
| 10 | R10 | | |
| Comparative | Comparative | 95% | 102% |
| Example 1- | Example Compound | | |
| 11 | R11 | | |
| Comparative | Comparative | 100% | 87% |
| Example 1- | Example Compound | | |
| 12 | R12 | | |
| Comparative | Comparative | 97% | 77% |
| Example 1- | Example Compound | | |
| 13 | R13 | | |
| Comparative | Comparative | 92% | 92% |
| Example 1- | Example Compound | | |
| 14 | R14 | | |
| Comparative | Comparative | 90% | 95% |
| Example 1- | Example Compound | | |
| 15 | R15 | | |
| Comparative | Comparative | 99% | 86% |
| Example 1- | Example Compound | | |
| 16 | R16 | | |
| Comparative | Comparative | 97% | 89% |
| Example 1- | Example Compound | | |
| 17 | R17 | | |
| Comparative | Comparative | 101% | 83% |
| Example 1- | Example Compound | | |
| 18 | R18 | | |
| Comparative | Comparative | 96% | 78% |
| Example 1- | Example Compound | | |

| | |
|---|---|
| 19 | R19 |

Referring to the results of Table 1, it may be seen that Examples of the light emitting elements utilizing the amine compounds of Examples according to the present disclosure as a hole transport layer material exhibit excellent or suitable luminous efficiency and long service life characteristics.

The amine compounds utilized in the light emitting elements of Examples 1-1 to 1-19 include a 2-carbazolyl group and two dibenzoheterole groups, and thus the high efficiency and long service life of the element are realized. For example, for the amine compounds of Examples, the carbazole group having a high hole transport property with respect to the amine is bonded to the nitrogen atom moiety at the second position of the carbazole group, and thus the HOMO orbital is greatly extended extended. Accordingly, radical or radical cation species may be stabilized. In some embodiments, the amine compounds of Examples have two dibenzoheterole groups at the side chain of the amine, and thus the hole transport property is increased and the electron resistance is improved. Thus, the amine compounds of Examples may have realized specific high efficiency and long service life of the light emitting element.

Comparative Example Compound utilized in Comparative Example 1-1 is a material having only a single dibenzofuran group unlike Example Compounds, and the hole transport property and electron resistance are not sufficient, and thus both (e.g., simultaneously) the efficiency and the service life of the Comparative Example are reduced compared with Examples.

Comparative Example Compounds utilized in Comparative Examples 1-2, 1-3, 1-18, and 1-19 are materials having a different bonding position of the carbazole group from the compounds of Examples, and the HOMO orbital is difficult to be extended compared with the materials utilized in Examples. Accordingly, the radical or radical cation species may not be sufficiently stabilized, and thus, for example, the service lives of the Comparative Examples are reduced compared with Examples.

Comparative Example Compounds utilized in Comparative Examples 1-4 and 1-5 are materials in which the dibenzoheterole group is directly bonded to the nitrogen atom at the second position, and show the results in that both (e.g., simultaneously) the luminous efficiency and the service life are reduced compared with Examples. Because the heteroatom and the nitrogen atom in the dibenzoheterole skeleton are located at the para position with the benzene ring located therebetween, it is postulated that the radical or radical cation species is unstabilized, leading to the deterioration of materials during the driving by electrification. Compared with this, Examples utilizing the amine compounds in which the dibenzohelerole group is bonded to the nitrogen atom at the second position with a linking group located therebetween such as Examples Compounds A17, C38, D35, and D54 have improved stability, thereby exhibiting excellent or suitable element characteristics.

Comparative Example Compounds utilized in Comparative Examples 1-6 and 1-7 are compounds in which heterocyclic structures are further condensed with respect to the dibenzoheterole ring and at least two aromatic rings are condensed. Comparative Example Compounds utilized in Comparative Examples 1-8 and 1-9 are compounds in which an aryl group having many ring-forming carbon atoms is bonded at the ninth position of the carbazole group. Comparative Example Compound utilized in Comparative Example 1-10 is a compound in which both (e.g., simultaneously) the carbazole group and two dibenzoheterole groups is bonded to the nitrogen atom with a linker located therebetween. Comparative Example Compounds utilized in Comparative Examples 1-11 to 1-13 are compounds having a branched structure in which an aromatic group or a heterocyclic group is substituted at the linking group moiety for linking the carbazole group or the dibenzoheterole group with the nitrogen atom. Comparative Examples 1-6 to 1-13 all show results of reduction in both (e.g., simultaneously) the luminous efficiency and the service life compared with Examples. It is postulated that this is because the deposition temperature of the material is elevated, and thus the decomposition of materials is progressed under the high temperature condition.

Comparative Example Compounds utilized in Comparative Examples 1-14 to 1-17 are compounds having a plurality of amine moieties in substantially the same molecule, and show the results in that both (e.g., simultaneously) the luminous efficiency and the service life are reduced compared with Examples due to the collapse of the carrier balance.

TABLE 2

| Examples of | Electron | Luminous | Element |
|---|---|---|---|
| elements | blocking | efficiency | service life |
| manufactured | layer | @10 mA/cm² | LT50 |
| Example 2-1 | Example Compound | 150% | 158% |
| | A2 | | |
| Example 2-2 | Example Compound | 151% | 152% |
| | A17 | | |
| Example 2-3 | Example Compound | 159% | 150% |
| | A69 | | |
| Example 2-4 | Example Compound | 140% | 167% |
| | A90 | | |
| Example 2-5 | Example Compound | 143% | 166% |
| | B4 | | |
| Example 2-6 | Example Compound | 152% | 152% |
| | B36 | | |
| Example 2-7 | Example Compound | 150% | 156% |
| | B43 | | |
| Example 2-8 | Example Compound | 147% | 158% |
| | B86 | | |
| Example 2-9 | Example Compound | 140% | 160% |
| | C38 | | |
| Example 2- | Example Compound | 141% | 162% |
| 10 | C61 | | |
| Example 2- | Example Compound | 144% | 159% |
| 11 | C76 | | |
| Example 2- | Example Compound | 147% | 157% |
| 12 | C79 | | |
| Example 2- | Example Compound | 149% | 154% |
| 13 | D19 | | |
| Example 2- | Example Compound | 155% | 151% |
| 14 | D35 | | |
| Example 2- | Example Compound | 151% | 159% |
| 15 | D54 | | |
| Example 2- | Example Compound | 150% | 163% |
| 16 | D81 | | |
| Example 2- | Example Compound | 148% | 165% |
| 17 | A39 | | |
| Example 2- | Example Compound | 147% | 159% |
| 18 | B40 | | |
| Example 2- | Example Compound | 151% | 148% |
| 19 | C83 | | |
| Comparative | Comparative | 100% | 100% |
| Example 2-1 | Example Compound | | |
| | R1 | | |
| Comparative | Comparative | 107% | 81% |
| Example 2-2 | Example Compound | | |
| | R2 | | |
| Comparative | Comparative | 103% | 91% |
| Example 2-3 | Example Compound | | |
| | R3 | | |
| Comparative | Comparative | 110% | 102% |
| Example 2-4 | Example Compound | | |
| | R4 | | |
| Comparative | Comparative | 112% | 104% |
| Example 2-5 | Example Compound | | |
| | R5 | | |
| Comparative | Comparative | 102% | 79% |
| Example 2-6 | Example Compound | | |
| | R6 | | |
| Comparative | Comparative | 100% | 85% |
| Example 2-7 | Example Compound | | |
| | R7 | | |
| Comparative | Comparative | 113% | 95% |
| Example 2-8 | Example Compound | | |
| | R8 | | |
| Comparative | Comparative | 90% | 71% |
| Example 2-9 | Example Compound | | |
| | R9 | | |
| Comparative | Comparative | 117% | 68% |
| Example 2- | Example Compound | | |
| 10 | R10 | | |
| Comparative | Comparative | 98% | 107% |
| Example 2- | Example Compound | | |
| 11 | R11 | | |
| Comparative | Comparative | 103% | 95% |
| Example 2- | Example Compound | | |
| 12 | R12 | | |
| Comparative | Comparative | 99% | 87% |
| Example 2- | Example Compound | | |
| 13 | R13 | | |
| Comparative | Comparative | 94% | 99% |
| Example 2- | Example Compound | | |
| 14 | R14 | | |
| Comparative | Comparative | 91% | 104% |
| Example 2- | Example Compound | | |
| 15 | R15 | | |
| Comparative | Comparative | 94% | 96% |
| Example 2- | Example Compound | | |
| 16 | R16 | | |
| Comparative | Comparative | 98% | 98% |
| Example 2- | Example Compound | | |
| 17 | R17 | | |
| Comparative | Comparative | 102% | 89% |
| Example 2- | Example Compound | | |
| 18 | R18 | | |
| Comparative | Comparative | 96% | 79% |
| Example 2- | Example Compound | | |
| 19 | R19 | | |

Referring to the results of Table 2, it may be confirmed that the light emitting elements of Examples 2-1 to 2-19 exhibit long service life and high efficiency characteristics compared with those of Comparative Examples 2-1 to 2-19. For example, it may be seen that even when the amine compound of an example is utilized in the electron blocking layer, the light emitting element may exhibit excellent or suitable device characteristics.

Thus, the compounds utilized in Examples may improve luminous efficiency and luminous service life at the same time compared with the compound utilized in Comparative Examples. For example, for the amine compounds of Examples, the carbazole moiety is bonded to the amine moiety at the second position of the carbazole, and two dibenzoheterole moieties are bonded via a specific linker to the nitrogen atom of the amine or directly bonded at a specific position, thereby making it possible to improve both (e.g., simultaneously) the efficiency and service life of the light emitting element when utilized as a luminescent material.

The light emitting element of an embodiment may include the amine compound of an embodiment, thereby exhibiting high efficiency and long service life characteristics.

The amine compound of an embodiment may be utilized to achieve improved characteristics of the light emitting element having high efficiency and a long service life.

The use of “may” when describing embodiments of the present disclosure refers to “one or more embodiments of the present disclosure.”

As used herein, the term “substantially,” “about,” and similar terms are used as terms of approximation and not as terms of degree, and are intended to account for the inherent deviations in measured or calculated values that would be recognized by those of ordinary skill in the art. “About” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this disclosure is intended to include all higher numerical limitations subsumed therein. Accordingly, Applicant reserves the right to amend this disclosure, including the claims, to expressly recite any sub-range subsumed within the ranges expressly recited herein.

The light emitting device or any other relevant devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

Although the embodiments of the present disclosure have been described, it is understood that the present disclosure should not be limited to these embodiments, but one or more suitable changes and modifications can be made by one ordinary skilled in the art within the spirit and scope of the present present disclosure as defined by the following claims and equivalents thereof.

What is claimed is:

1. A light emitting element comprising:
a first electrode;
a second electrode on the first electrode; and
at least one functional layer which is between the first electrode and the second electrode and comprises an amine compound represented by Formula 1: 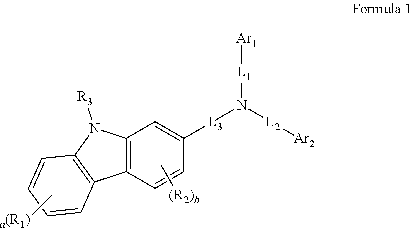
wherein, in Formula 1,
R₁ and R₂ are each independently a hydrogen atom, a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, and/or are bonded to an adjacent group to form a ring,
when R₁ and R₂ are each a substituted silyl group, a substituted alkyl group, a substituted alkenyl group, or a substituted aryl group, the substituent is not an amine group,
R₃ is an unsubstituted aryl group having 6 to 10 ring-forming carbon atoms, or an aryl group having 6 to 10 ring-forming carbon atoms substituted with a deuterium atom, a halogen atom, or a substituted or unsubstituted alkyl group having 1 to 20 carbon atoms,
a is an integer from 0 to 4, and b is an integer from 0 to 3,
at least one selected from among L₁ to L₃ is a direct linkage, and the L₁ to L₃ that are not a direct linkage are represented by any one selected from among Formula 2-1 to Formula 2-3, and
Ar₁ and Ar₂ are each independently represented by Formula 3: 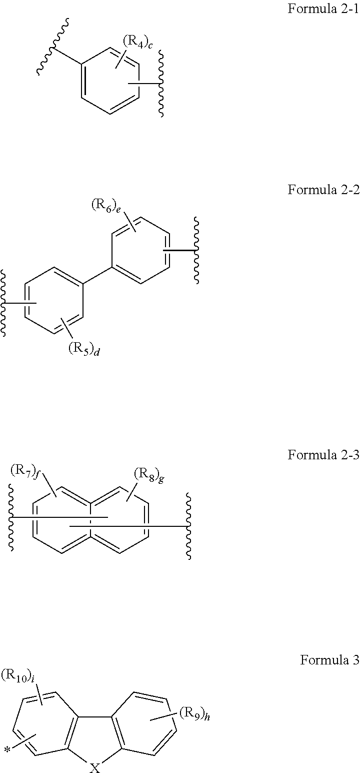
wherein, in Formula 2-1 to Formula 2-3,
R₄ to R₈ are each independently a hydrogen atom, a deuterium atom, a halogen atom, or a substituted or unsubstituted alkyl group having 1 to 20 carbon atoms, and
c, d, e, f, and g are each independently an integer from 0 to 4, and
in Formula 3,
X is O or S,
R₉ and R₁₀ are each independently a hydrogen atom, a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, and/or adjacent R₉s or adjacent R₁₀′ are bonded to each other to form an aromatic hydrocarbon ring,
when adjacent R₉s or adjacent R₁₀s are bonded to each other to form an aromatic hydrocarbon ring, only one pair of R₉s or R₁₀s selected from among a plurality of R₉s and R₁₀s are bonded to each other to form an aromatic hydrocarbon ring,
h is an integer from 0 to 4, and i is an integer from 0 to 3,
“-*” is a position linked to L₁ and L₂, and
when L₁ and L₂ are each a direct linkage, “-*” is not linked to a nitrogen atom in the para relation to X.

2. The light emitting element of claim 1, wherein the at least one functional layer comprises an emission layer, a hole transport region between the first electrode and the emission layer, and an electron transport region between the emission layer and the second electrode, and
the hole transport region comprises the amine compound.

3. The light emitting element of claim 2, wherein the hole transport region comprises at least one of a hole injection layer, a hole transport layer, or an electron blocking layer, and
at least one of the hole transport layer or the electron blocking layer comprises the amine compound.

4. The light emitting element of claim 1, wherein R₁ and R₂ are each independently a hydrogen atom or a deuterium atom, and/or are bonded to an adjacent group to form an aromatic hydrocarbon ring.

5. The light emitting element of claim 1, wherein R₃ is represented by any one selected from among Substituent Group S1: 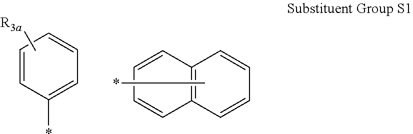
wherein, in Substituent Group S1,
R₃ₐ is a hydrogen atom, a deuterium atom, an alkyl group having 1 to 10 carbon atoms, or a halogen atom.

6. The light emitting element of claim 1, wherein Formula 3 is represented by Formula 3-1 or Formula 3-2: 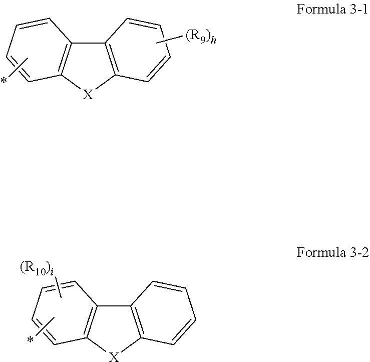
wherein, in Formula 3-1 and Formula 3-2,
R₉, R₁₀, X, h, and i are the same as defined in Formula 3.

7. The light emitting element of claim 1, wherein Formula 3 is represented by any one selected from among 3-a to 3-k:

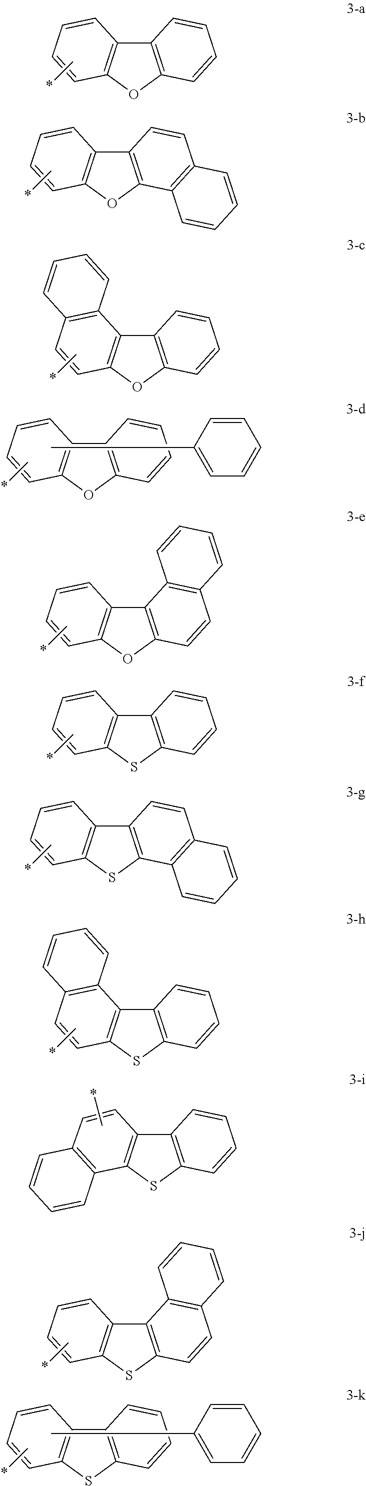

8. The light emitting element of claim 1, wherein at least one selected from among L₁ to L₃ is a direct linkage, and the L₁ to L₃ that are not a direct linkage are represented by any one selected from among Substituent Group S2:

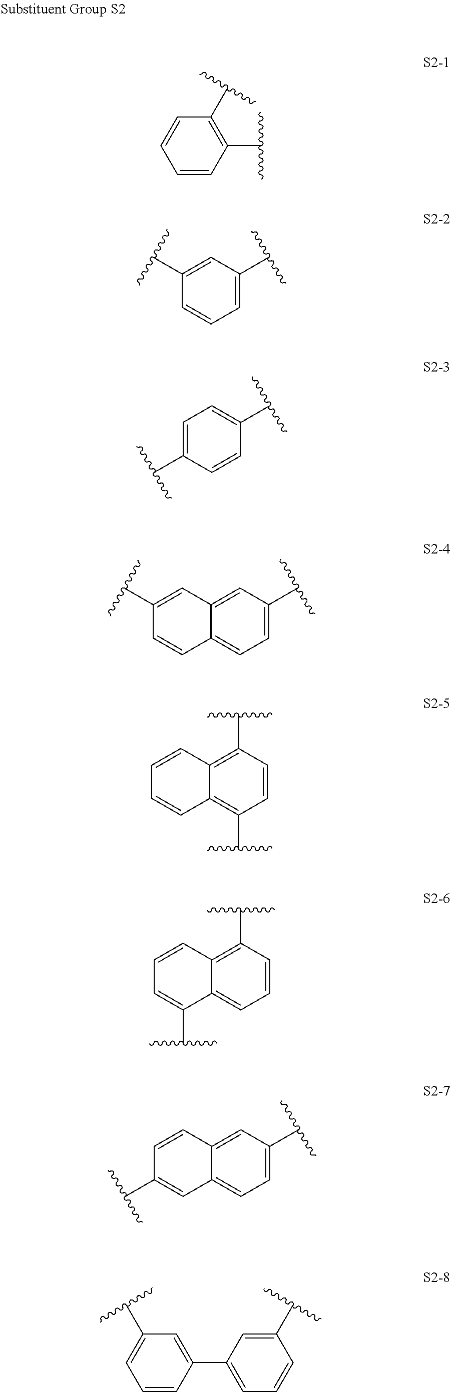

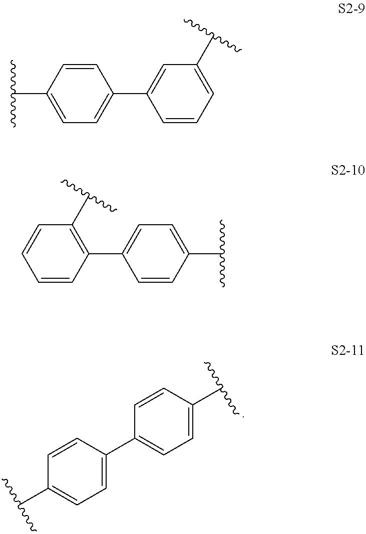 [SMILES: *c1ccc(-c2cccc(*)c2)cc1 ; *c1ccc(-c2ccccc2*)cc1 ; *c1ccc(-c2ccc(*)cc2)cc1]

9. The light emitting element of claim 1, wherein Formula 1 is represented by Formula 4: 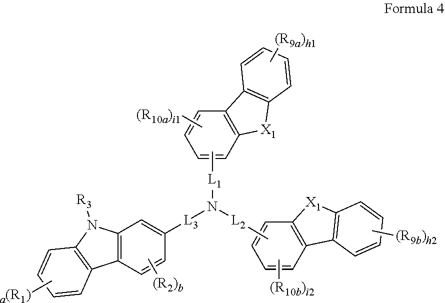
wherein, in Formula 4,
X₁ and X₂ are each independently O or S,
R₉ₐ and R₁₀ₐ are each independently a hydrogen atom, a deuterium atom, or a substituted or unsubstituted aryl group having 6 to 30 ring-forming carbon atoms, and/or adjacent R₉ₐs or adjacent R₁₀ₐs are bonded to each other to form an aromatic ring,
R_{9b} and R_{10b} are each independently a hydrogen atom, a deuterium atom, or a substituted or unsubstituted aryl group having 6 to 30 ring-forming carbon atoms, or adjacent R_{9b}s or adjacent R_{10b}s are bonded to each other to form an aromatic ring,
h1 and h2 are each independently an integer from 0 to 4,
i1 and i2 are each independently an integer from 0 to 3, and
R₁ to R₃, a, b, and L₁ to L₃ are the same as defined in Formula 1.

10. The light emitting element of claim 9, wherein Formula 4 is represented by any one selected from among Formula 4-1 to Formula 4-5: 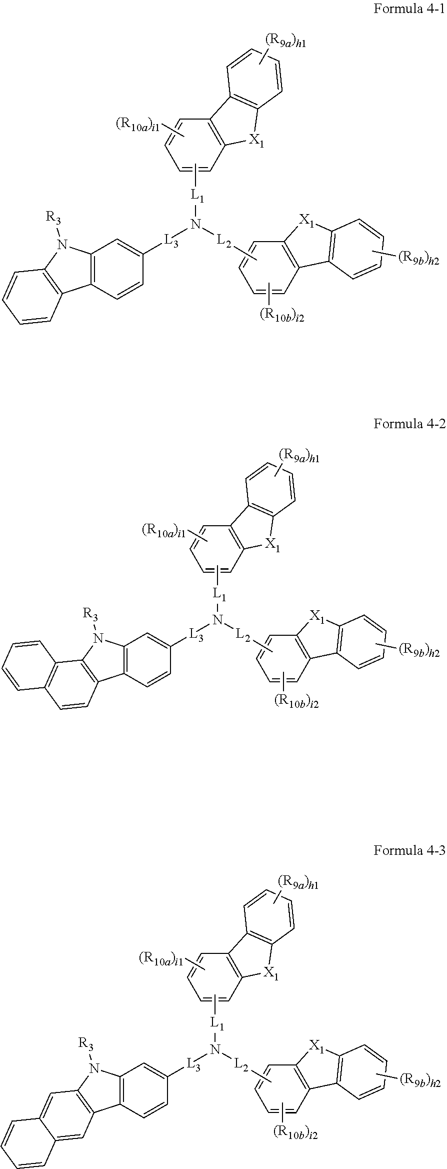 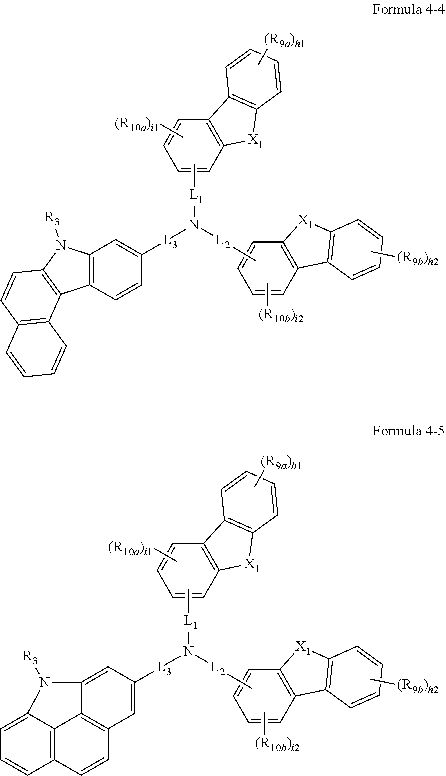
wherein, in Formula 4-1 to Formula 4-5,
R₃, and L₁ to L₃ are the same as defined in Formula 1, and
X₁, X₂, R₉ₐ, R_{9b}, R₁₀ₐ, R_{10b}, h1, h2, i1, and i2 are the same as defined in Formula 4.

11. The light emitting element of claim 2, wherein the emission layer comprises a compound represented by Formula E-1: 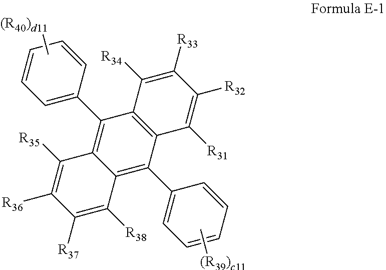
wherein, in Formula E-1, R₃₁ to R₄₀ are each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or are bonded to an adjacent group to form a ring, and
c11 and d11 are each independently an integer from 0 to 5.

12. The light emitting element of claim 1, wherein the amine compound is any one selected from among compounds of Compound Group 1:

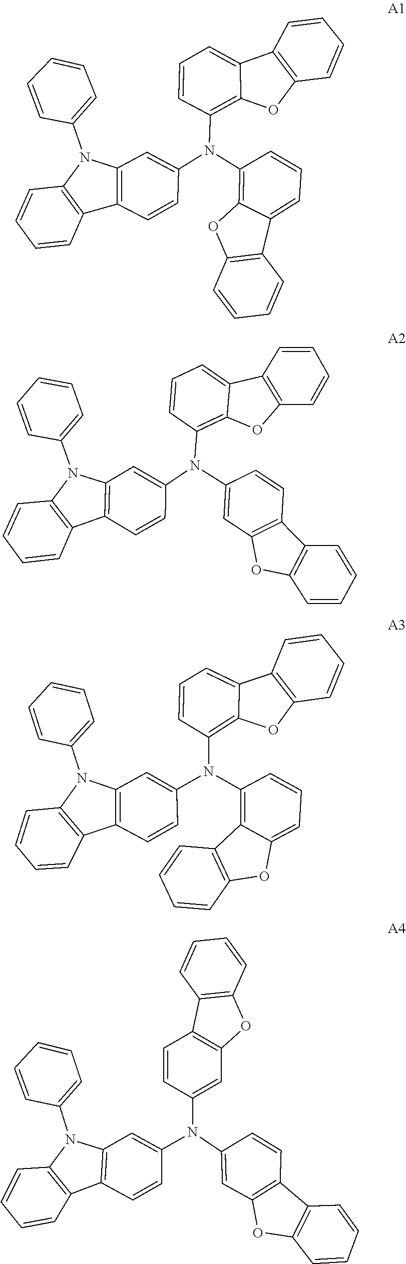

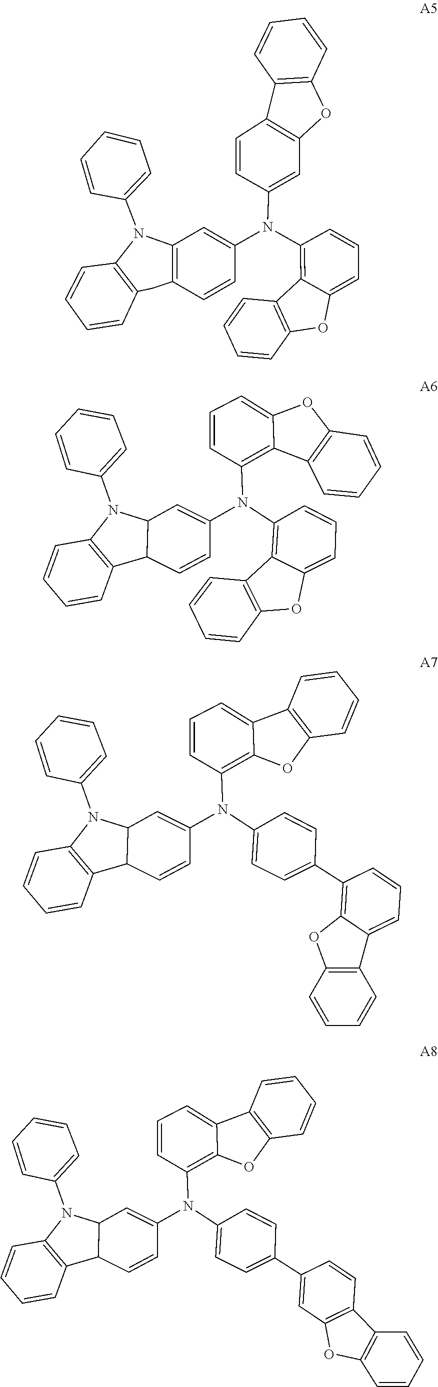

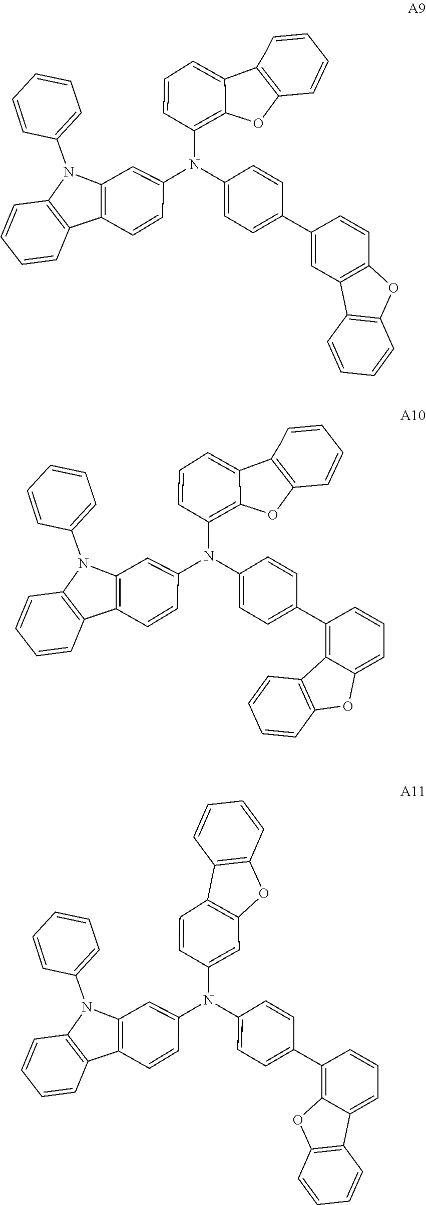 [SMILES: C1=CCc2c(oc3c(N(c4ccc(-c5ccc6oc7ccccc7c6c5)cc4)c4ccc5c6ccccc6n(-c6ccccc6)c5c4)cccc23)C=C1 ; C1=CCc2c(oc3c(N(c4ccc(-c5cccc6oc7ccccc7c56)cc4)c4ccc5c6ccccc6n(-c6ccccc6)c5c4)cccc23)C=C1 ; c1ccc(-n2c3ccccc3c3ccc(N(c4ccc(-c5cccc6c5oc5ccccc56)cc4)c4ccc5c(c4)oc4ccccc45)cc32)cc1]

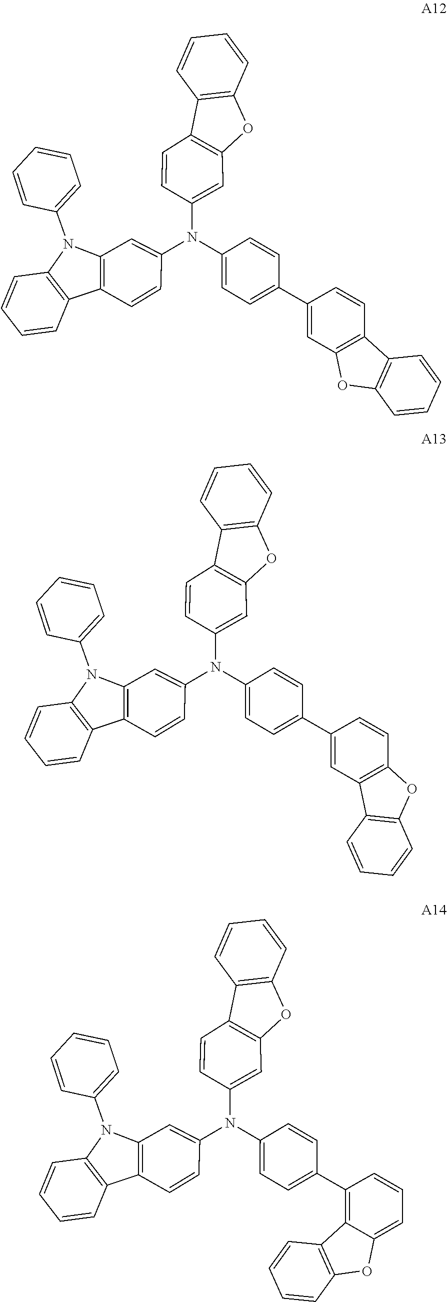 [SMILES: c1ccc(-n2c3ccccc3c3ccc(N(c4ccc(-c5ccc6c(c5)oc5ccccc56)cc4)c4ccc5c(c4)oc4ccccc45)cc32)cc1 ; c1ccc(-n2c3ccccc3c3ccc(N(c4ccc(-c5ccc6oc7ccccc7c6c5)cc4)c4ccc5c(c4)oc4ccccc45)cc32)cc1 ; c1ccc(-n2c3ccccc3c3ccc(N(c4ccc(-c5cccc6oc7ccccc7c56)cc4)c4ccc5c(c4)oc4ccccc45)cc32)cc1]

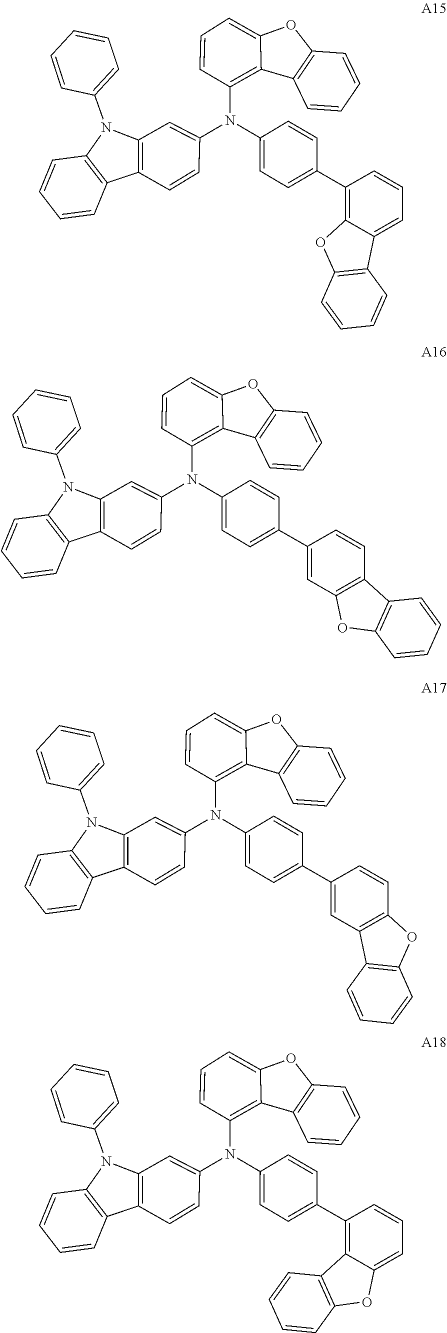 [SMILES: c1ccc(-n2c3ccccc3c3ccc(N(c4ccc(-c5cccc6c5oc5ccccc56)cc4)c4cccc5oc6ccccc6c45)cc32)cc1 ; c1ccc(-n2c3ccccc3c3ccc(N(c4ccc(-c5ccc6c(c5)oc5ccccc56)cc4)c4cccc5oc6ccccc6c45)cc32)cc1 ; c1ccc(-n2c3ccccc3c3ccc(N(c4ccc(-c5ccc6oc7ccccc7c6c5)cc4)c4cccc5oc6ccccc6c45)cc32)cc1 ; c1ccc(-n2c3ccccc3c3ccc(N(c4ccc(-c5cccc6oc7ccccc7c56)cc4)c4cccc5oc6ccccc6c45)cc32)cc1]

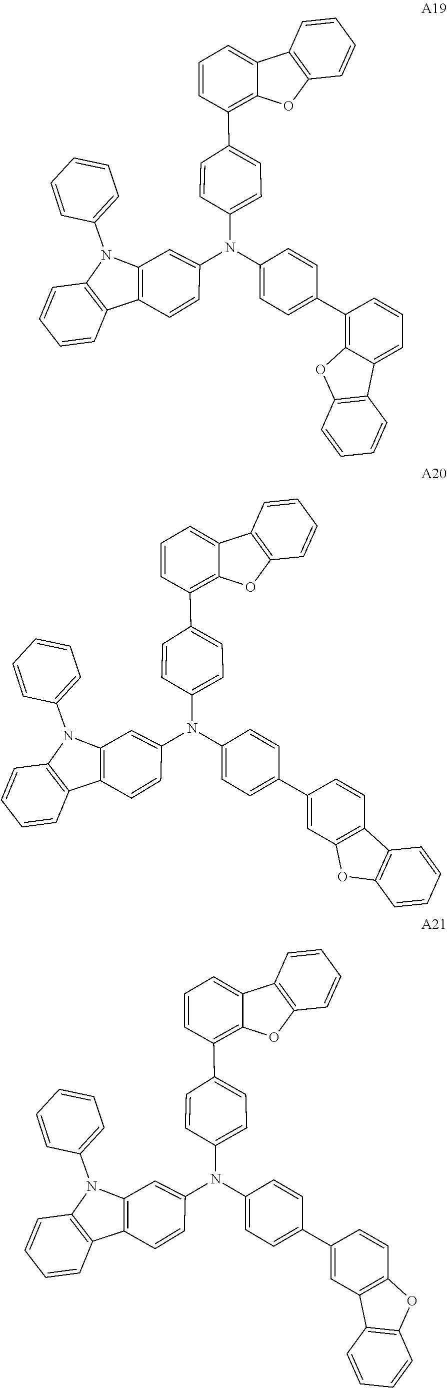 [SMILES: c1ccc(-n2c3ccccc3c3ccc(N(c4ccc(-c5cccc6c5oc5ccccc56)cc4)c4ccc(-c5cccc6c5oc5ccccc56)cc4)cc32)cc1 ; c1ccc(-n2c3ccccc3c3ccc(N(c4ccc(-c5ccc6c(c5)oc5ccccc56)cc4)c4ccc(-c5cccc6c5oc5ccccc56)cc4)cc32)cc1 ; c1ccc(-n2c3ccccc3c3ccc(N(c4ccc(-c5ccc6oc7ccccc7c6c5)cc4)c4ccc(-c5cccc6c5oc5ccccc56)cc4)cc32)cc1]

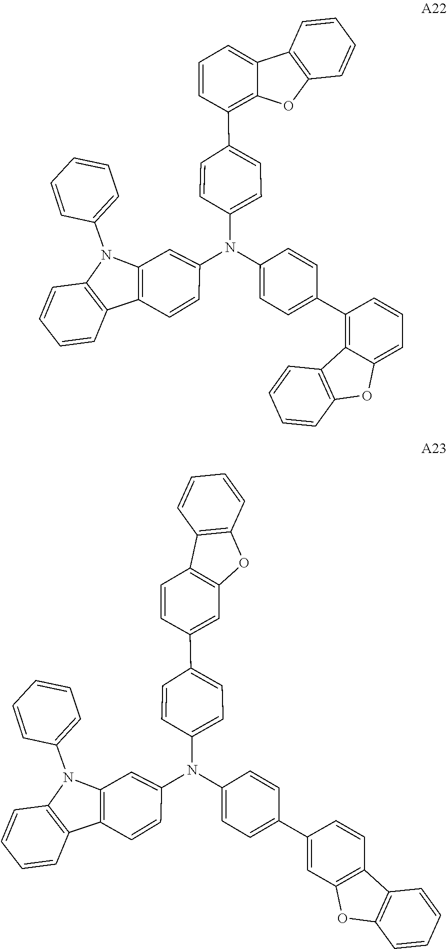 [SMILES: c1ccc(-n2c3ccccc3c3ccc(N(c4ccc(-c5cccc6c5oc5ccccc56)cc4)c4ccc(-c5cccc6oc7ccccc7c56)cc4)cc32)cc1 ; c1ccc(-n2c3ccccc3c3ccc(N(c4ccc(-c5ccc6c(c5)oc5ccccc56)cc4)c4ccc(-c5ccc6c(c5)oc5ccccc56)cc4)cc32)cc1]

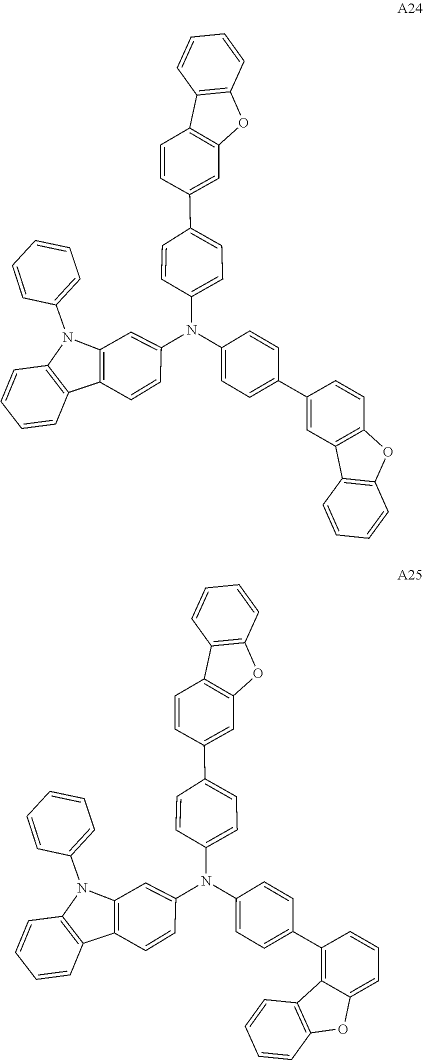 [SMILES: c1ccc(-n2c3ccccc3c3ccc(N(c4ccc(-c5ccc6c(c5)oc5ccccc56)cc4)c4ccc(-c5ccc6oc7ccccc7c6c5)cc4)cc32)cc1 ; c1ccc(-n2c3ccccc3c3ccc(N(c4ccc(-c5ccc6c(c5)oc5ccccc56)cc4)c4ccc(-c5cccc6oc7ccccc7c56)cc4)cc32)cc1]

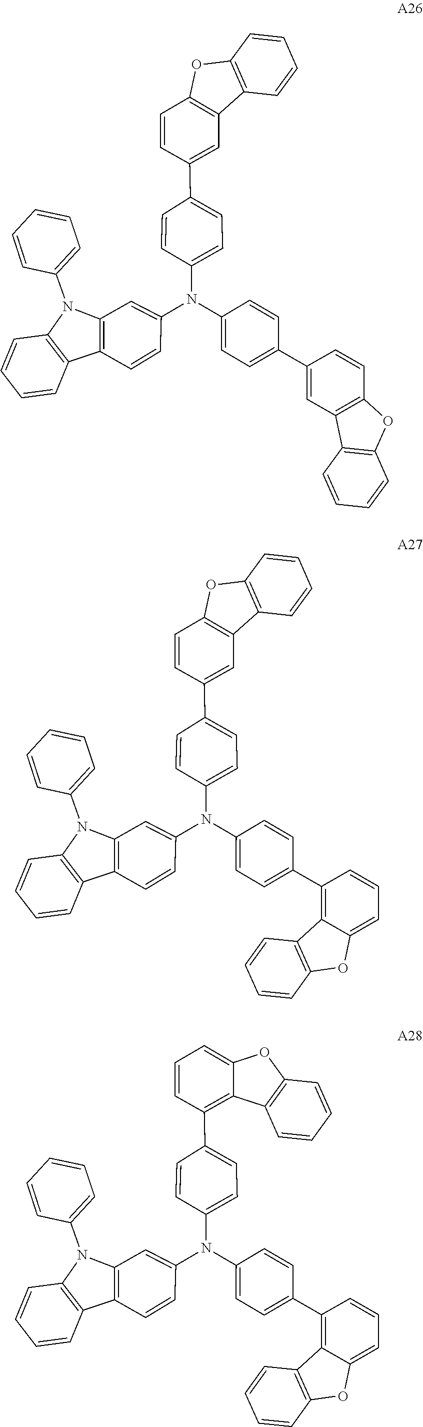 [SMILES: c1ccc(-n2c3ccccc3c3ccc(N(c4ccc(-c5ccc6oc7ccccc7c6c5)cc4)c4ccc(-c5ccc6oc7ccccc7c6c5)cc4)cc32)cc1 ; c1ccc(-n2c3ccccc3c3ccc(N(c4ccc(-c5ccc6oc7ccccc7c6c5)cc4)c4ccc(-c5cccc6oc7ccccc7c56)cc4)cc32)cc1 ; c1ccc(-n2c3ccccc3c3ccc(N(c4ccc(-c5cccc6oc7ccccc7c56)cc4)c4ccc(-c5cccc6oc7ccccc7c56)cc4)cc32)cc1]

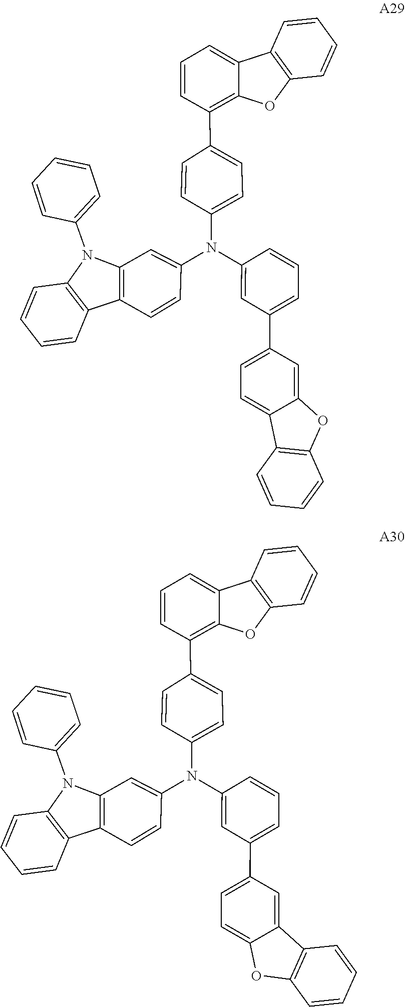

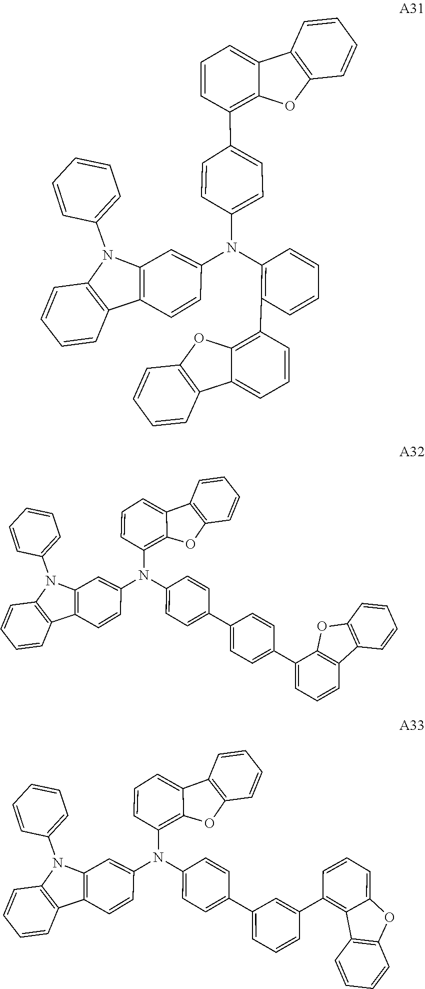 [SMILES: c1ccc(-n2c3ccccc3c3ccc(N(c4ccc(-c5cccc6c5oc5ccccc56)cc4)c4ccccc4-c4cccc5c4oc4ccccc45)cc32)cc1 ; c1ccc(-n2c3ccccc3c3ccc(N(c4ccc(-c5ccc(-c6cccc7c6oc6ccccc67)cc5)cc4)c4cccc5c4oc4ccccc45)cc32)cc1 ; c1ccc(-n2c3ccccc3c3ccc(N(c4ccc(-c5cccc(-c6cccc7oc8ccccc8c67)c5)cc4)c4cccc5c4oc4ccccc45)cc32)cc1]

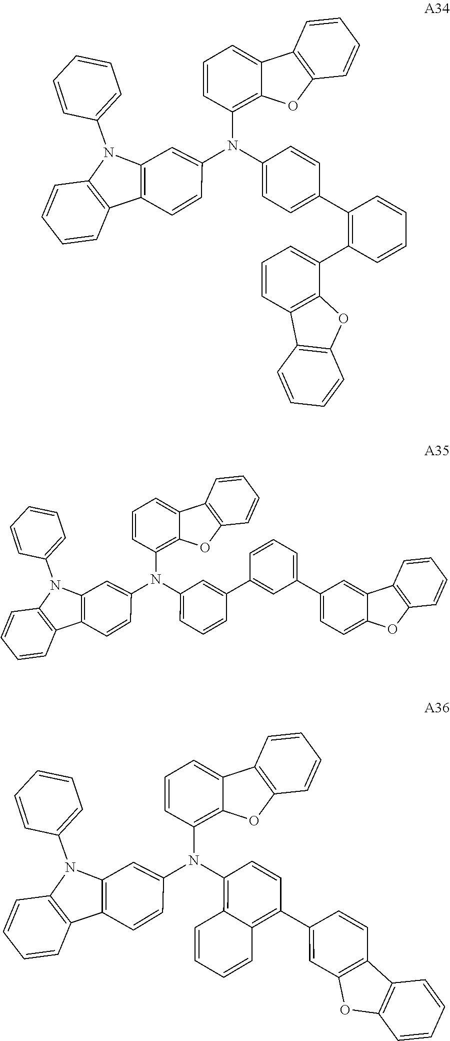 [SMILES: c1ccc(-n2c3ccccc3c3ccc(N(c4ccc(-c5ccccc5-c5cccc6c5oc5ccccc56)cc4)c4cccc5c4oc4ccccc45)cc32)cc1 ; c1ccc(-n2c3ccccc3c3ccc(N(c4cccc(-c5cccc(-c6ccc7oc8ccccc8c7c6)c5)c4)c4cccc5c4oc4ccccc45)cc32)cc1 ; c1ccc(-n2c3ccccc3c3ccc(N(c4ccc(-c5ccc6c(c5)oc5ccccc56)c5ccccc45)c4cccc5c4oc4ccccc45)cc32)cc1]

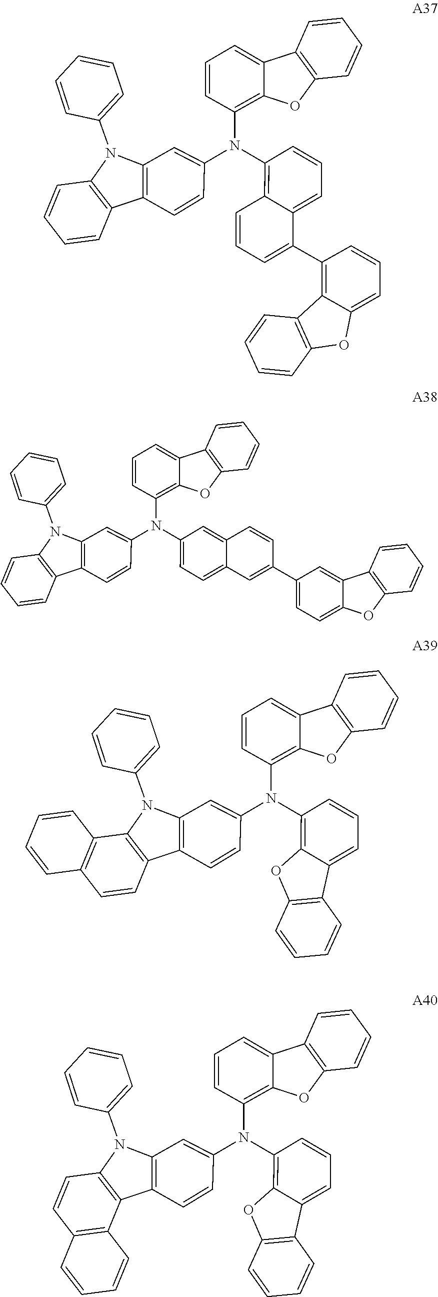 [SMILES: c1ccc(-n2c3ccccc3c3ccc(N(c4cccc5c(-c6cccc7oc8ccccc8c67)cccc45)c4cccc5c4oc4ccccc45)cc32)cc1 ; c1ccc(-n2c3ccccc3c3ccc(N(c4ccc5cc(-c6ccc7oc8ccccc8c7c6)ccc5c4)c4cccc5c4oc4ccccc45)cc32)cc1 ; c1ccc(-n2c3cc(N(c4cccc5c4oc4ccccc45)c4cccc5c4oc4ccccc45)ccc3c3ccc4ccccc4c32)cc1 ; c1ccc(-n2c3cc(N(c4cccc5c4oc4ccccc45)c4cccc5c4oc4ccccc45)ccc3c3c4ccccc4ccc32)cc1]

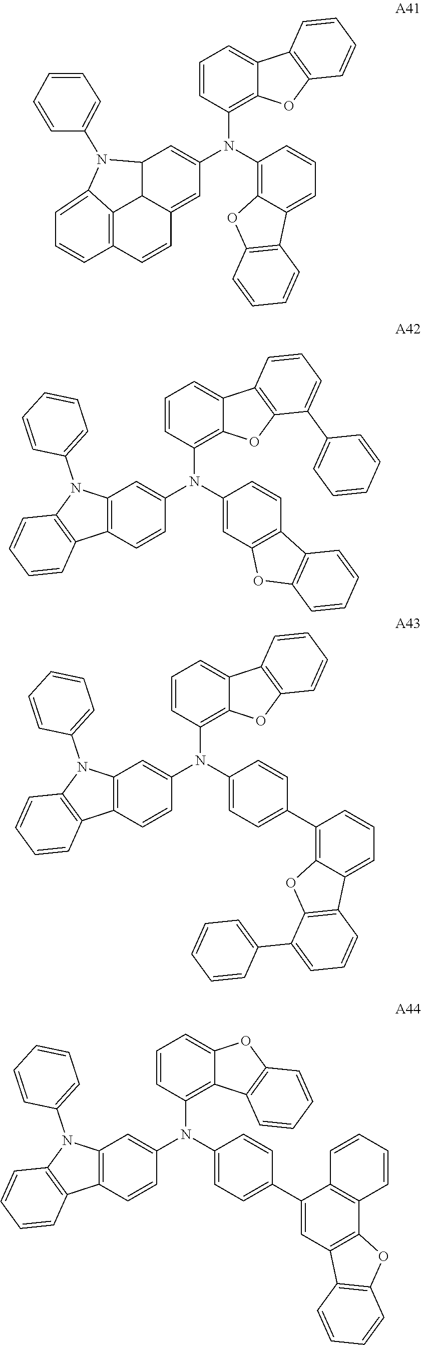

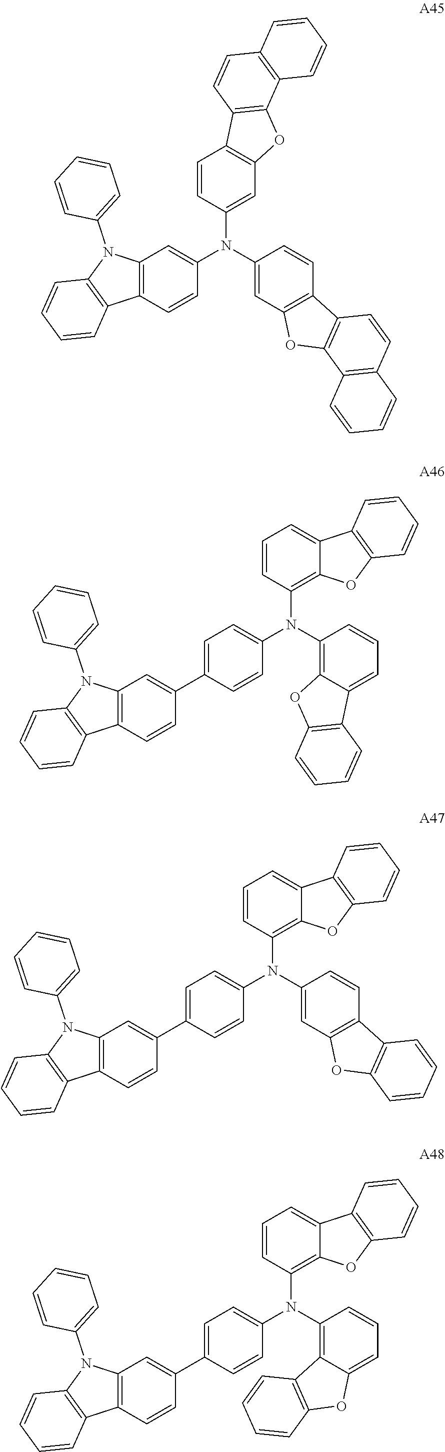 [SMILES: c1ccc(-n2c3ccccc3c3ccc(N(c4ccc5c(c4)oc4c6ccccc6ccc54)c4ccc5c(c4)oc4c6ccccc6ccc54)cc32)cc1 ; c1ccc(-n2c3ccccc3c3ccc(-c4ccc(N(c5cccc6c5oc5ccccc56)c5cccc6c5oc5ccccc56)cc4)cc32)cc1 ; c1ccc(-n2c3ccccc3c3ccc(-c4ccc(N(c5ccc6c(c5)oc5ccccc56)c5cccc6c5oc5ccccc56)cc4)cc32)cc1 ; c1ccc(-n2c3ccccc3c3ccc(-c4ccc(N(c5cccc6c5oc5ccccc56)c5cccc6oc7ccccc7c56)cc4)cc32)cc1]

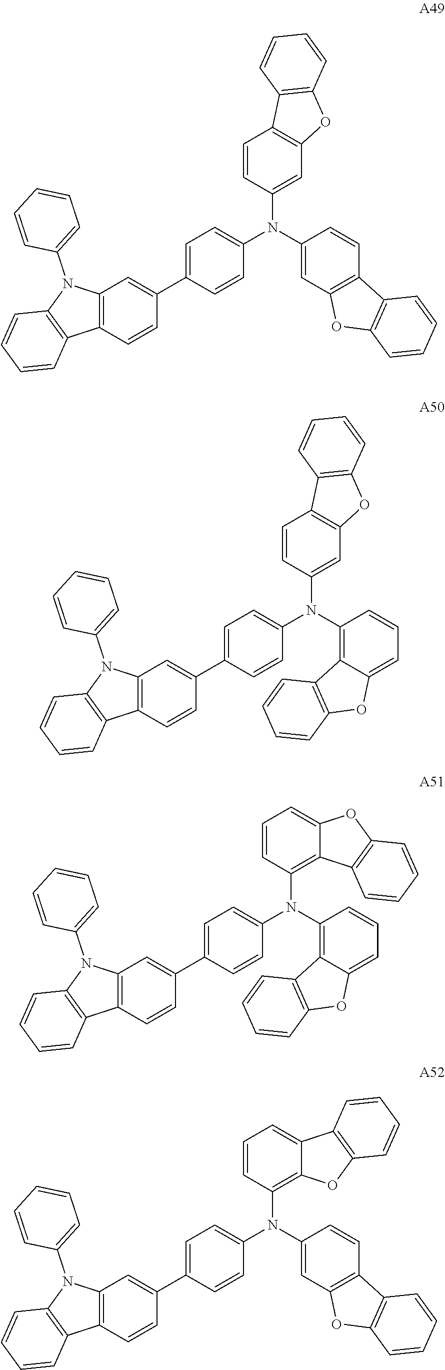 [SMILES: c1ccc(-n2c3ccccc3c3ccc(-c4ccc(N(c5ccc6c(c5)oc5ccccc56)c5ccc6c(c5)oc5ccccc56)cc4)cc32)cc1 ; c1ccc(-n2c3ccccc3c3ccc(-c4ccc(N(c5ccc6c(c5)oc5ccccc56)c5cccc6oc7ccccc7c56)cc4)cc32)cc1 ; c1ccc(-n2c3ccccc3c3ccc(-c4ccc(N(c5cccc6oc7ccccc7c56)c5cccc6oc7ccccc7c56)cc4)cc32)cc1 ; c1ccc(-n2c3ccccc3c3ccc(-c4ccc(N(c5ccc6c(c5)oc5ccccc56)c5cccc6c5oc5ccccc56)cc4)cc32)cc1]

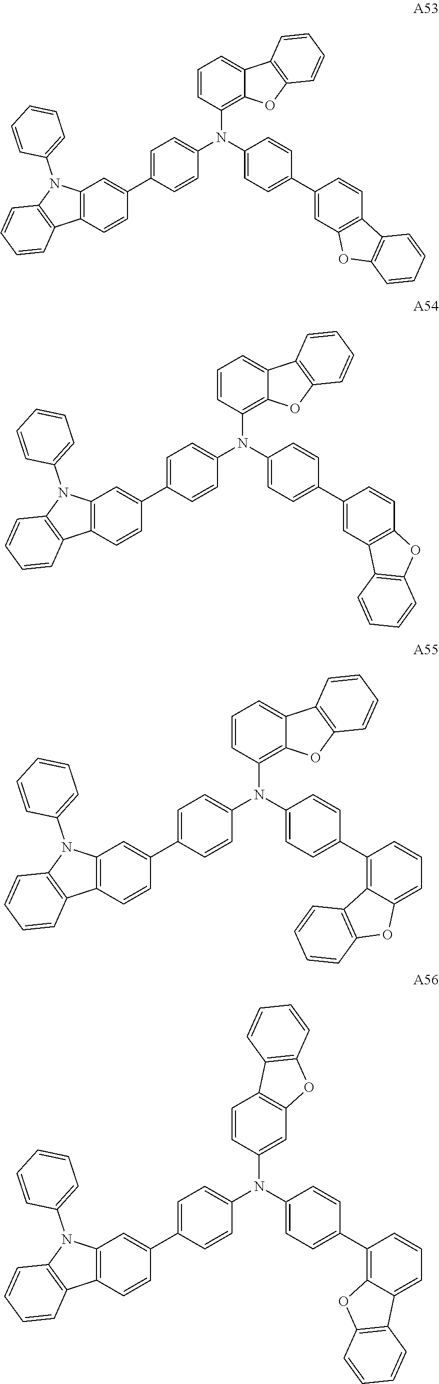 [SMILES: c1ccc(-n2c3ccccc3c3ccc(-c4ccc(N(c5ccc(-c6ccc7c(c6)oc6ccccc67)cc5)c5cccc6c5oc5ccccc56)cc4)cc32)cc1 ; c1ccc(-n2c3ccccc3c3ccc(-c4ccc(N(c5ccc(-c6ccc7oc8ccccc8c7c6)cc5)c5cccc6c5oc5ccccc56)cc4)cc32)cc1 ; c1ccc(-n2c3ccccc3c3ccc(-c4ccc(N(c5ccc(-c6cccc7oc8ccccc8c67)cc5)c5cccc6c5oc5ccccc56)cc4)cc32)cc1 ; c1ccc(-n2c3ccccc3c3ccc(-c4ccc(N(c5ccc(-c6cccc7c6oc6ccccc67)cc5)c5ccc6c(c5)oc5ccccc56)cc4)cc32)cc1]

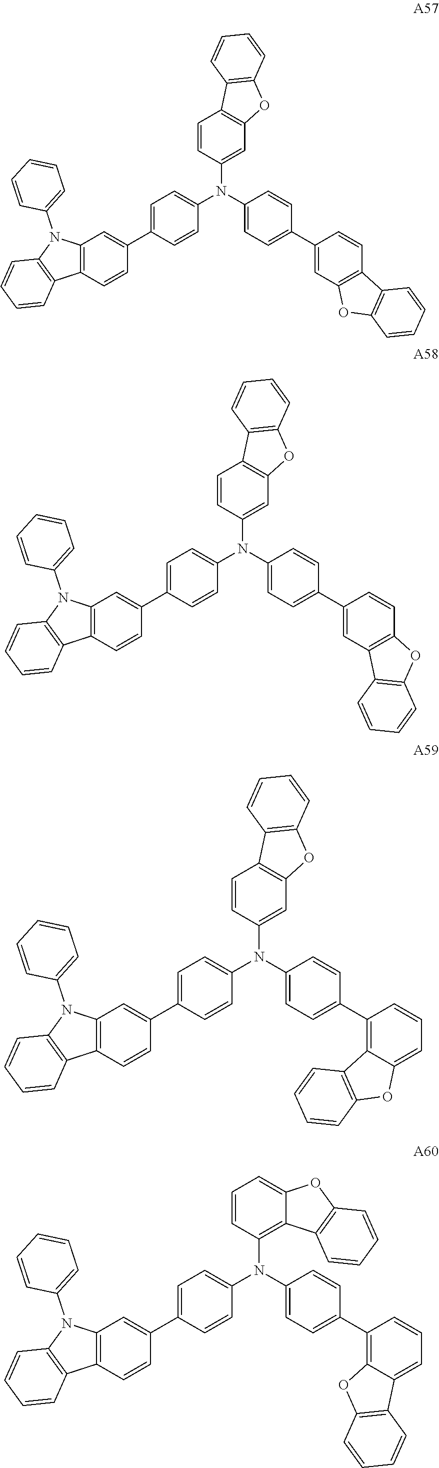 [SMILES: c1ccc(-n2c3ccccc3c3ccc(-c4ccc(N(c5ccc(-c6ccc7c(c6)oc6ccccc67)cc5)c5ccc6c(c5)oc5ccccc56)cc4)cc32)cc1 ; c1ccc(-n2c3ccccc3c3ccc(-c4ccc(N(c5ccc(-c6ccc7oc8ccccc8c7c6)cc5)c5ccc6c(c5)oc5ccccc56)cc4)cc32)cc1 ; c1ccc(-n2c3ccccc3c3ccc(-c4ccc(N(c5ccc(-c6cccc7oc8ccccc8c67)cc5)c5ccc6c(c5)oc5ccccc56)cc4)cc32)cc1 ; c1ccc(-n2c3ccccc3c3ccc(-c4ccc(N(c5ccc(-c6cccc7c6oc6ccccc67)cc5)c5cccc6oc7ccccc7c56)cc4)cc32)cc1]

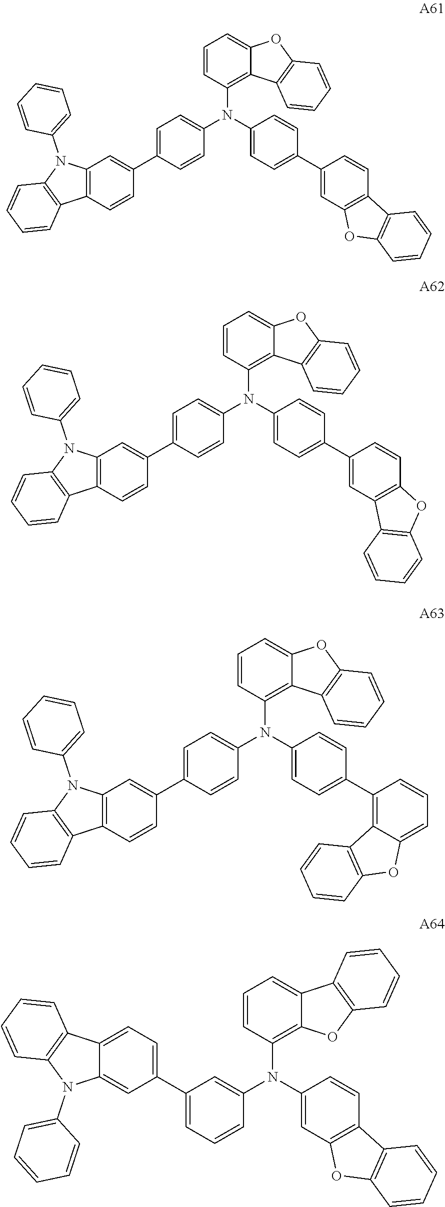 [SMILES: c1ccc(-n2c3ccccc3c3ccc(-c4ccc(N(c5ccc(-c6ccc7c(c6)oc6ccccc67)cc5)c5cccc6oc7ccccc7c56)cc4)cc32)cc1 ; c1ccc(-n2c3ccccc3c3ccc(-c4ccc(N(c5ccc(-c6ccc7oc8ccccc8c7c6)cc5)c5cccc6oc7ccccc7c56)cc4)cc32)cc1 ; c1ccc(-n2c3ccccc3c3ccc(-c4ccc(N(c5ccc(-c6cccc7oc8ccccc8c67)cc5)c5cccc6oc7ccccc7c56)cc4)cc32)cc1 ; c1ccc(-n2c3ccccc3c3ccc(-c4cccc(N(c5ccc6c(c5)oc5ccccc56)c5cccc6c5oc5ccccc56)c4)cc32)cc1]

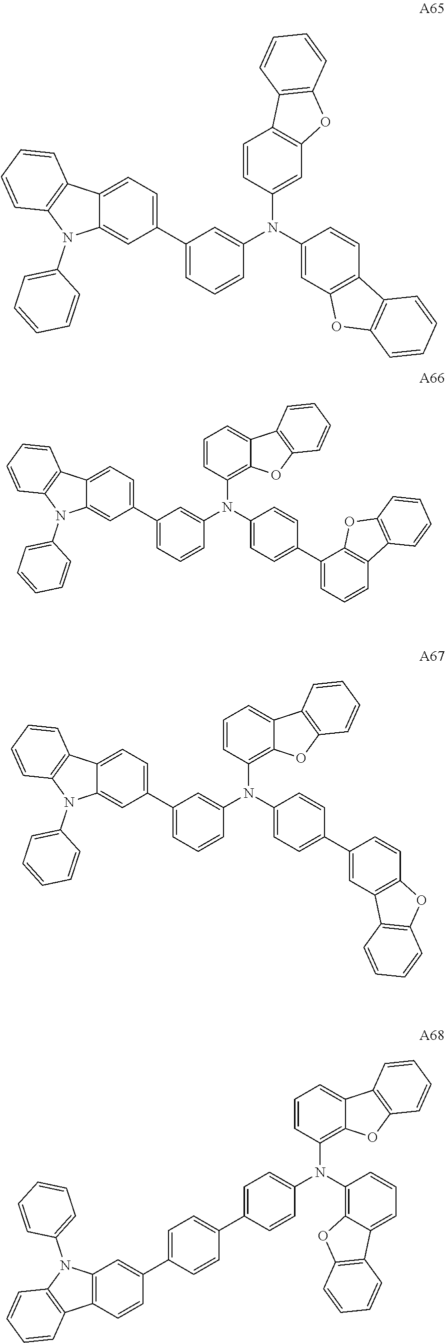 [SMILES: c1ccc(-n2c3ccccc3c3ccc(-c4cccc(N(c5ccc6c(c5)oc5ccccc56)c5ccc6c(c5)oc5ccccc56)c4)cc32)cc1 ; c1ccc(-n2c3ccccc3c3ccc(-c4cccc(N(c5ccc(-c6cccc7c6oc6ccccc67)cc5)c5cccc6c5oc5ccccc56)c4)cc32)cc1 ; c1ccc(-n2c3ccccc3c3ccc(-c4cccc(N(c5ccc(-c6ccc7oc8ccccc8c7c6)cc5)c5cccc6c5oc5ccccc56)c4)cc32)cc1 ; c1ccc(-n2c3ccccc3c3ccc(-c4ccc(-c5ccc(N(c6cccc7c6oc6ccccc67)c6cccc7c6oc6ccccc67)cc5)cc4)cc32)cc1]

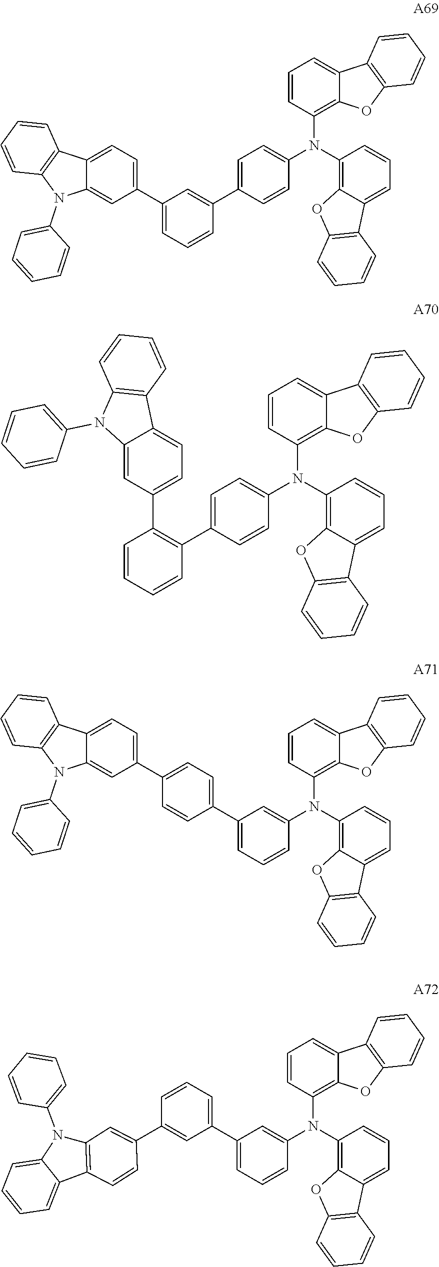 [SMILES: c1ccc(-n2c3ccccc3c3ccc(-c4cccc(-c5ccc(N(c6cccc7c6oc6ccccc67)c6cccc7c6oc6ccccc67)cc5)c4)cc32)cc1 ; c1ccc(-n2c3ccccc3c3ccc(-c4ccccc4-c4ccc(N(c5cccc6c5oc5ccccc56)c5cccc6c5oc5ccccc56)cc4)cc32)cc1 ; c1ccc(-n2c3ccccc3c3ccc(-c4ccc(-c5cccc(N(c6cccc7c6oc6ccccc67)c6cccc7c6oc6ccccc67)c5)cc4)cc32)cc1 ; c1ccc(-n2c3ccccc3c3ccc(-c4cccc(-c5cccc(N(c6cccc7c6oc6ccccc67)c6cccc7c6oc6ccccc67)c5)c4)cc32)cc1]

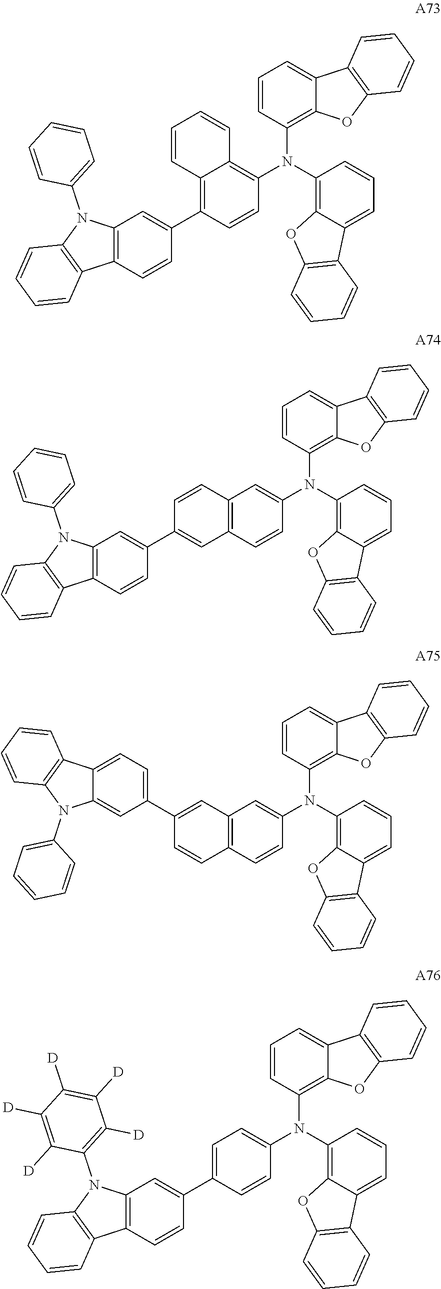 [SMILES: c1ccc(-n2c3ccccc3c3ccc(-c4ccc(N(c5cccc6c5oc5ccccc56)c5cccc6c5oc5ccccc56)c5ccccc45)cc32)cc1 ; c1ccc(-n2c3ccccc3c3ccc(-c4ccc5cc(N(c6cccc7c6oc6ccccc67)c6cccc7c6oc6ccccc67)ccc5c4)cc32)cc1 ; c1ccc(-n2c3ccccc3c3ccc(-c4ccc5ccc(N(c6cccc7c6oc6ccccc67)c6cccc7c6oc6ccccc67)cc5c4)cc32)cc1 ; [2H]c1c([2H])c([2H])c(-n2c3ccccc3c3ccc(-c4ccc(N(c5cccc6c5oc5ccccc56)c5cccc6c5oc5ccccc56)cc4)cc32)c([2H])c1[2H]]

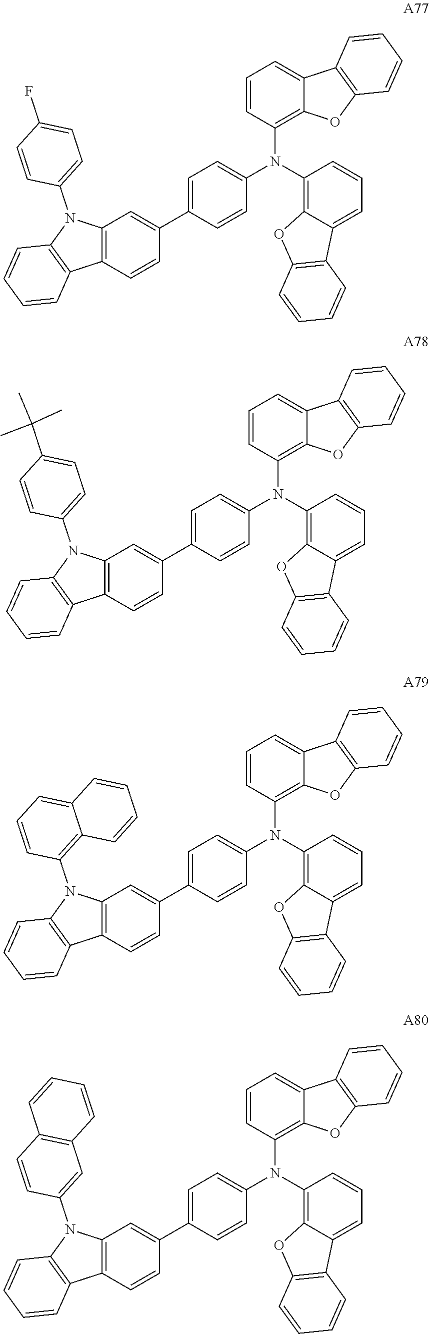 [SMILES: Fc1ccc(-n2c3ccccc3c3ccc(-c4ccc(N(c5cccc6c5oc5ccccc56)c5cccc6c5oc5ccccc56)cc4)cc32)cc1 ; CC(C)(C)c1ccc(-n2c3ccccc3c3ccc(-c4ccc(N(c5cccc6c5oc5ccccc56)c5cccc6c5oc5ccccc56)cc4)cc32)cc1 ; c1ccc2c(-n3c4ccccc4c4ccc(-c5ccc(N(c6cccc7c6oc6ccccc67)c6cccc7c6oc6ccccc67)cc5)cc43)cccc2c1 ; c1ccc2cc(-n3c4ccccc4c4ccc(-c5ccc(N(c6cccc7c6oc6ccccc67)c6cccc7c6oc6ccccc67)cc5)cc43)ccc2c1]

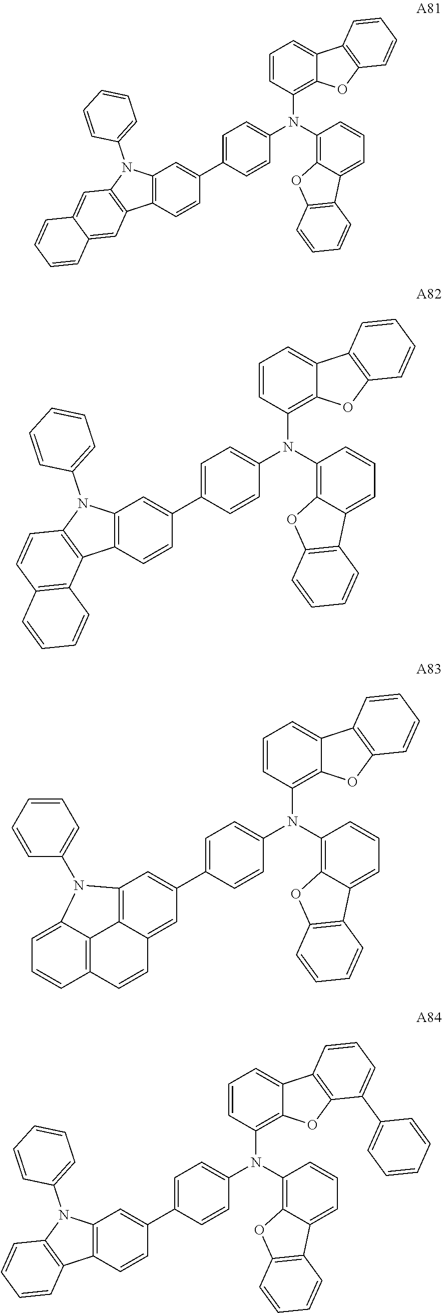 [SMILES: c1ccc(-n2c3cc(-c4ccc(N(c5cccc6c5oc5ccccc56)c5cccc6c5oc5ccccc56)cc4)ccc3c3cc4ccccc4cc32)cc1 ; c1ccc(-n2c3cc(-c4ccc(N(c5cccc6c5oc5ccccc56)c5cccc6c5oc5ccccc56)cc4)ccc3c3c4ccccc4ccc32)cc1 ; c1ccc(-n2c3cccc4ccc5cc(-c6ccc(N(c7cccc8c7oc7ccccc78)c7cccc8c7oc7ccccc78)cc6)cc2c5c43)cc1 ; c1ccc(-c2cccc3c2oc2c(N(c4ccc(-c5ccc6c7ccccc7n(-c7ccccc7)c6c5)cc4)c4cccc5c4oc4ccccc45)cccc23)cc1]

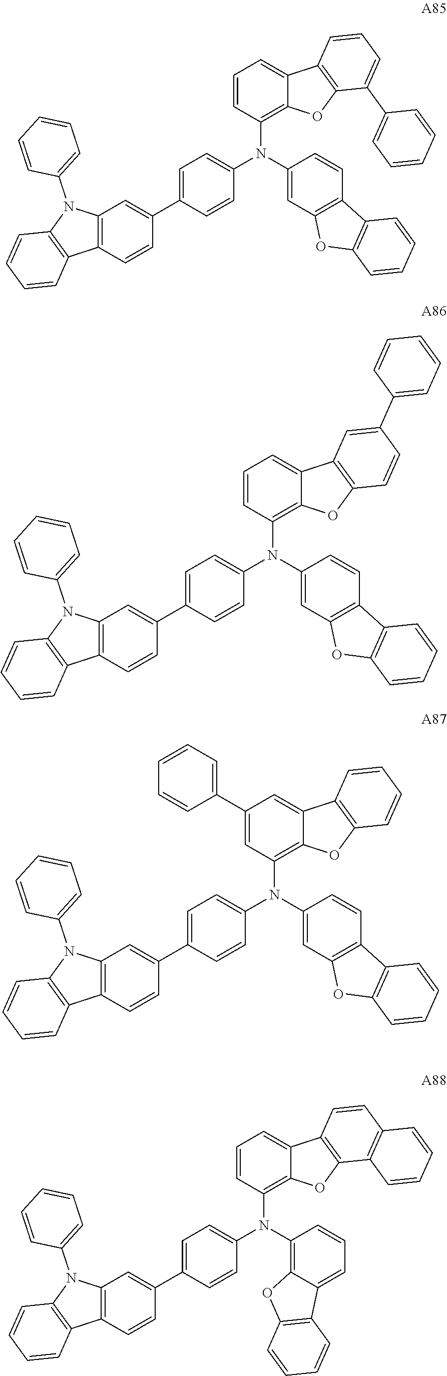 [SMILES: c1ccc(-c2cccc3c2oc2c(N(c4ccc(-c5ccc6c7ccccc7n(-c7ccccc7)c6c5)cc4)c4ccc5c(c4)oc4ccccc45)cccc23)cc1 ; c1ccc(-c2ccc3oc4c(N(c5ccc(-c6ccc7c8ccccc8n(-c8ccccc8)c7c6)cc5)c5ccc6c(c5)oc5ccccc56)cccc4c3c2)cc1 ; c1ccc(-c2cc(N(c3ccc(-c4ccc5c6ccccc6n(-c6ccccc6)c5c4)cc3)c3ccc4c(c3)oc3ccccc34)c3oc4ccccc4c3c2)cc1 ; c1ccc(-n2c3ccccc3c3ccc(-c4ccc(N(c5cccc6c5oc5ccccc56)c5cccc6c5oc5c7ccccc7ccc65)cc4)cc32)cc1]

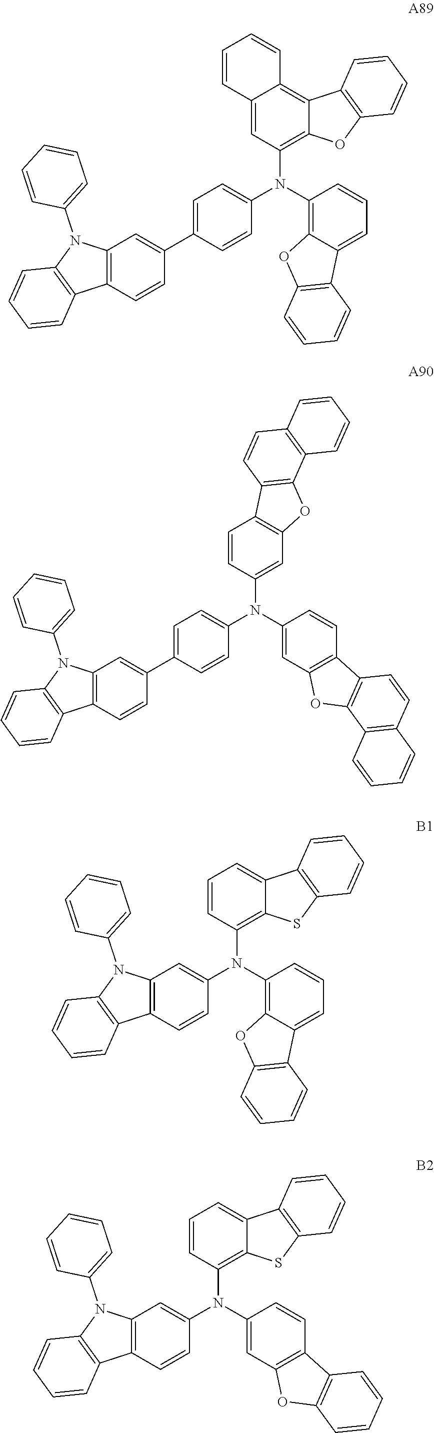 [SMILES: c1ccc(-n2c3ccccc3c3ccc(-c4ccc(N(c5cccc6c5oc5ccccc56)c5cc6ccccc6c6c5oc5ccccc56)cc4)cc32)cc1 ; c1ccc(-n2c3ccccc3c3ccc(-c4ccc(N(c5ccc6c(c5)oc5c7ccccc7ccc65)c5ccc6c(c5)oc5c7ccccc7ccc65)cc4)cc32)cc1 ; c1ccc(-n2c3ccccc3c3ccc(N(c4cccc5c4oc4ccccc45)c4cccc5c4sc4ccccc45)cc32)cc1 ; c1ccc(-n2c3ccccc3c3ccc(N(c4ccc5c(c4)oc4ccccc45)c4cccc5c4sc4ccccc45)cc32)cc1]

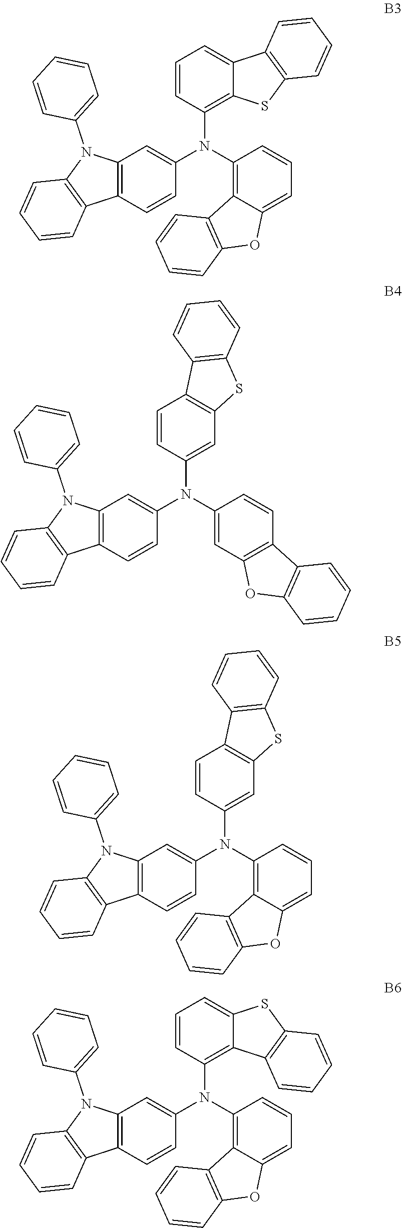

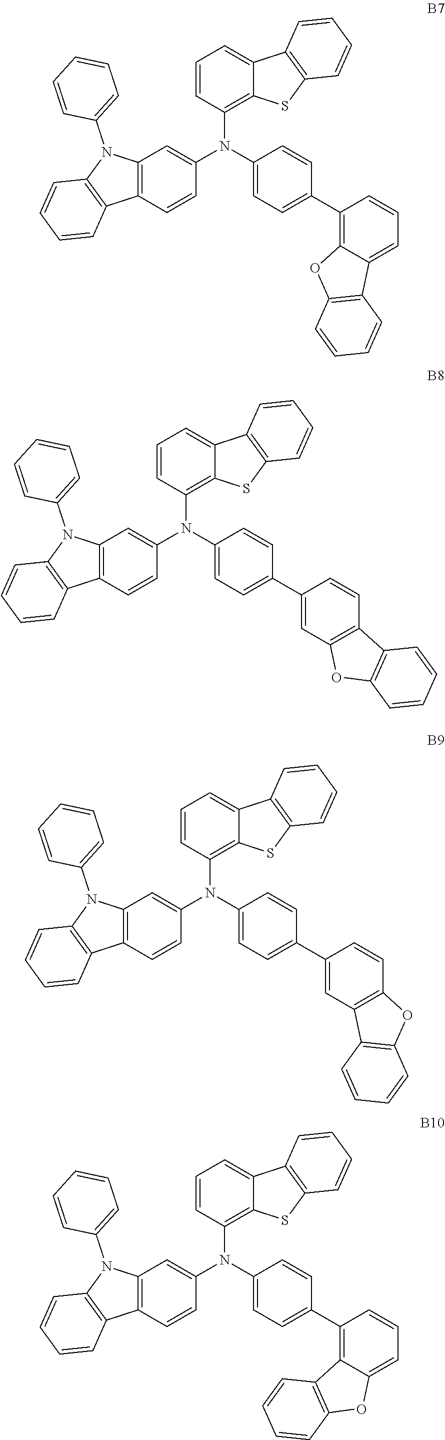

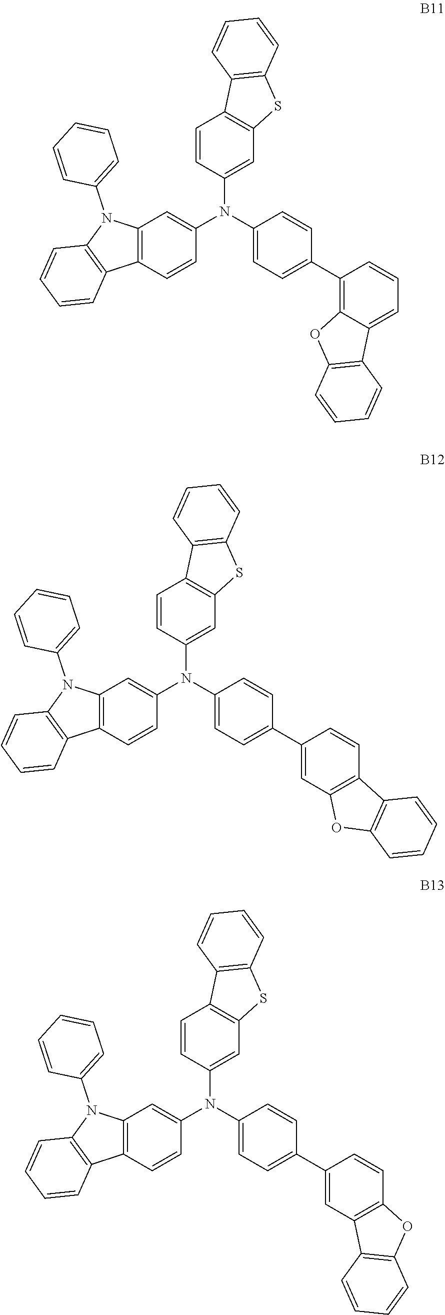 [SMILES: c1ccc(-n2c3ccccc3c3ccc(N(c4ccc(-c5cccc6c5oc5ccccc56)cc4)c4ccc5c(c4)sc4ccccc45)cc32)cc1 ; c1ccc(-n2c3ccccc3c3ccc(N(c4ccc(-c5ccc6c(c5)oc5ccccc56)cc4)c4ccc5c(c4)sc4ccccc45)cc32)cc1 ; c1ccc(-n2c3ccccc3c3ccc(N(c4ccc(-c5ccc6oc7ccccc7c6c5)cc4)c4ccc5c(c4)sc4ccccc45)cc32)cc1]

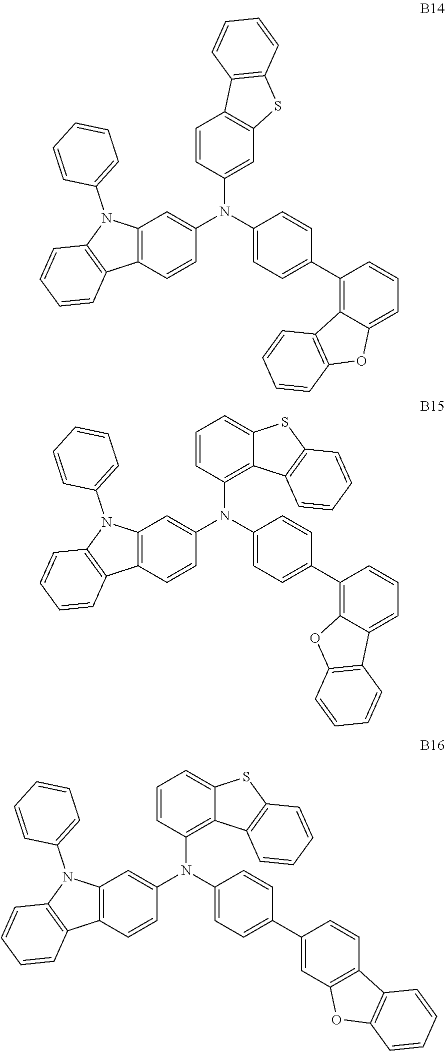

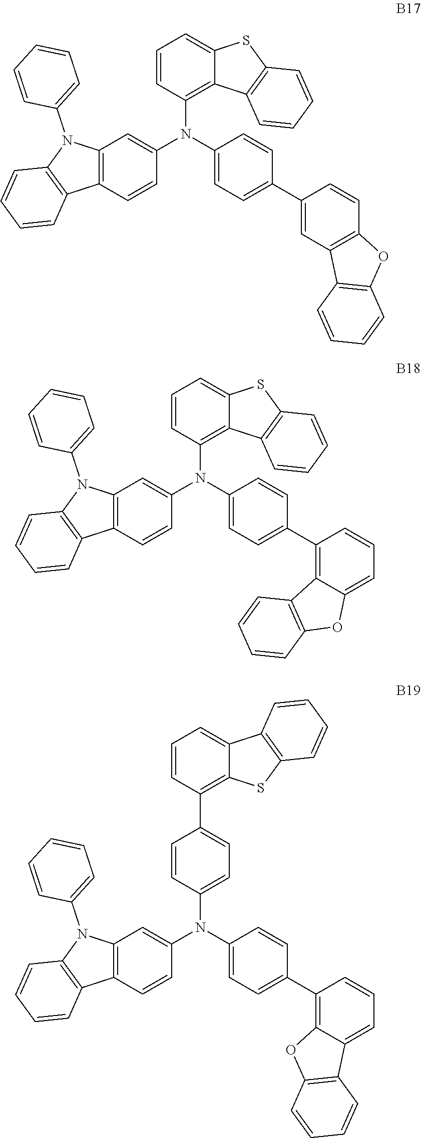

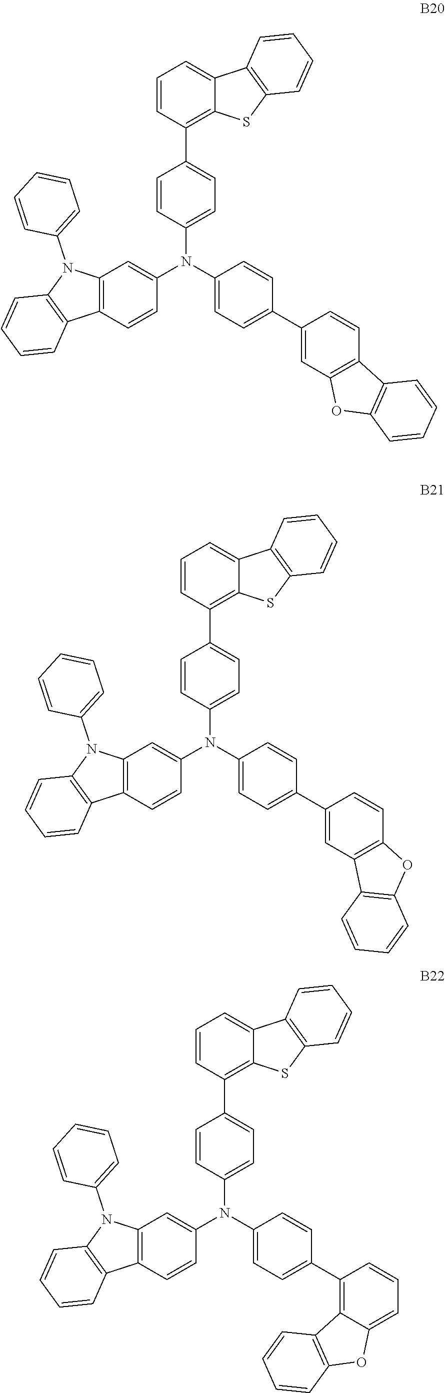

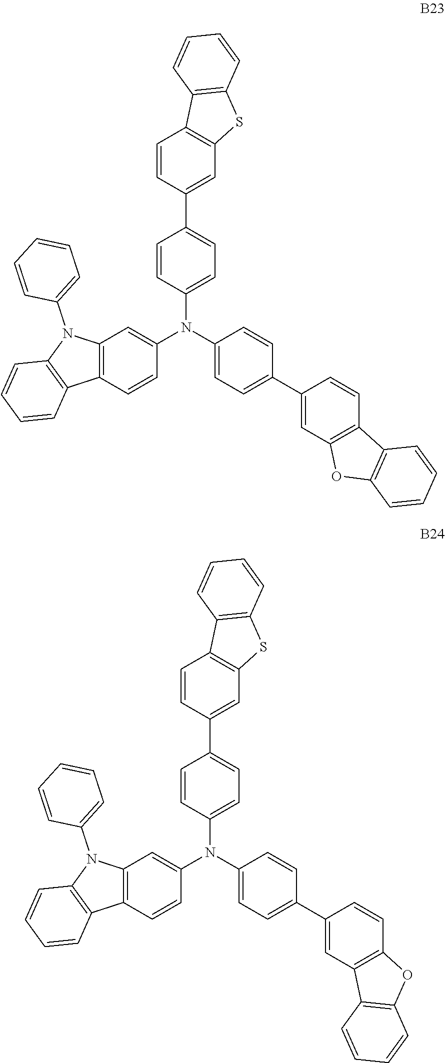 [SMILES: c1ccc(-n2c3ccccc3c3ccc(N(c4ccc(-c5ccc6c(c5)oc5ccccc56)cc4)c4ccc(-c5ccc6c(c5)sc5ccccc56)cc4)cc32)cc1 ; c1ccc(-n2c3ccccc3c3ccc(N(c4ccc(-c5ccc6c(c5)sc5ccccc56)cc4)c4ccc(-c5ccc6oc7ccccc7c6c5)cc4)cc32)cc1]

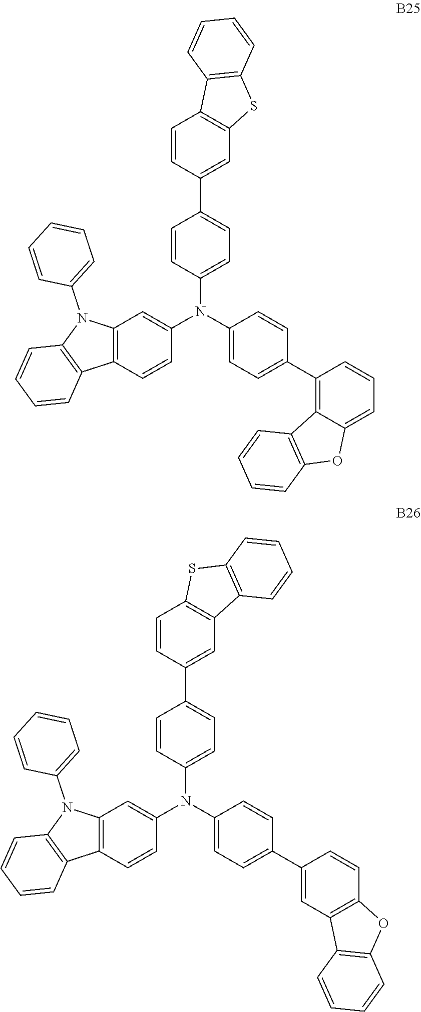 [SMILES: c1ccc(-n2c3ccccc3c3ccc(N(c4ccc(-c5ccc6c(c5)sc5ccccc56)cc4)c4ccc(-c5cccc6oc7ccccc7c56)cc4)cc32)cc1 ; c1ccc(-n2c3ccccc3c3ccc(N(c4ccc(-c5ccc6oc7ccccc7c6c5)cc4)c4ccc(-c5ccc6sc7ccccc7c6c5)cc4)cc32)cc1]

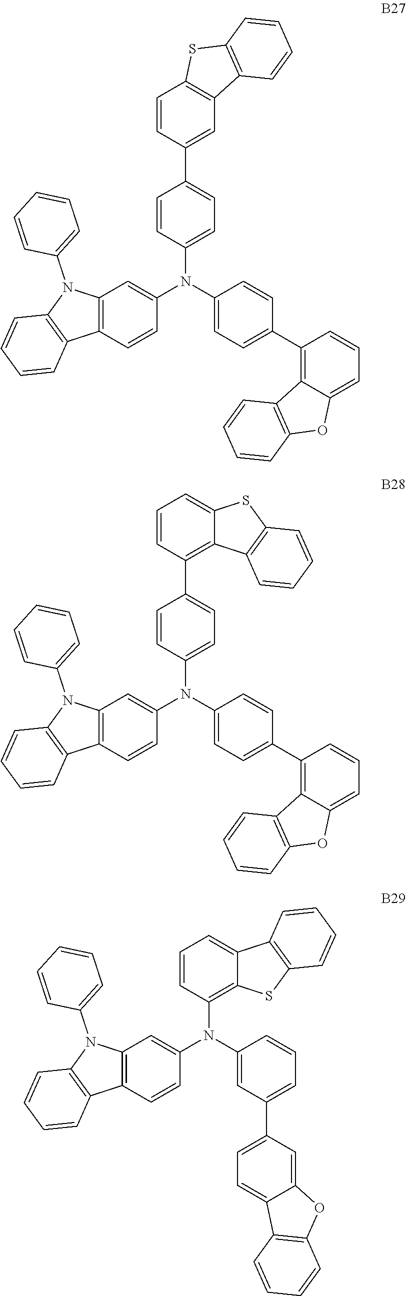

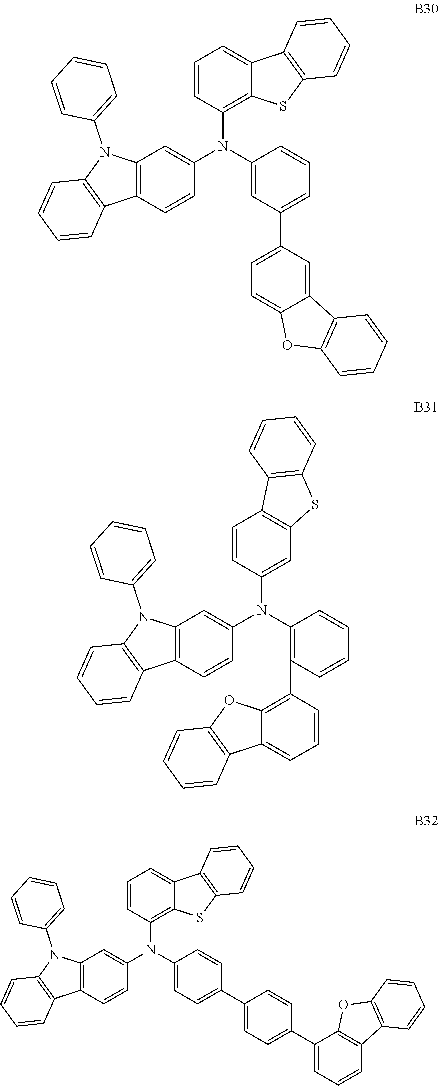 [SMILES: c1ccc(-n2c3ccccc3c3ccc(N(c4cccc(-c5ccc6oc7ccccc7c6c5)c4)c4cccc5c4sc4ccccc45)cc32)cc1 ; c1ccc(-n2c3ccccc3c3ccc(N(c4ccc5c(c4)sc4ccccc45)c4ccccc4-c4cccc5c4oc4ccccc45)cc32)cc1 ; c1ccc(-n2c3ccccc3c3ccc(N(c4ccc(-c5ccc(-c6cccc7c6oc6ccccc67)cc5)cc4)c4cccc5c4sc4ccccc45)cc32)cc1]

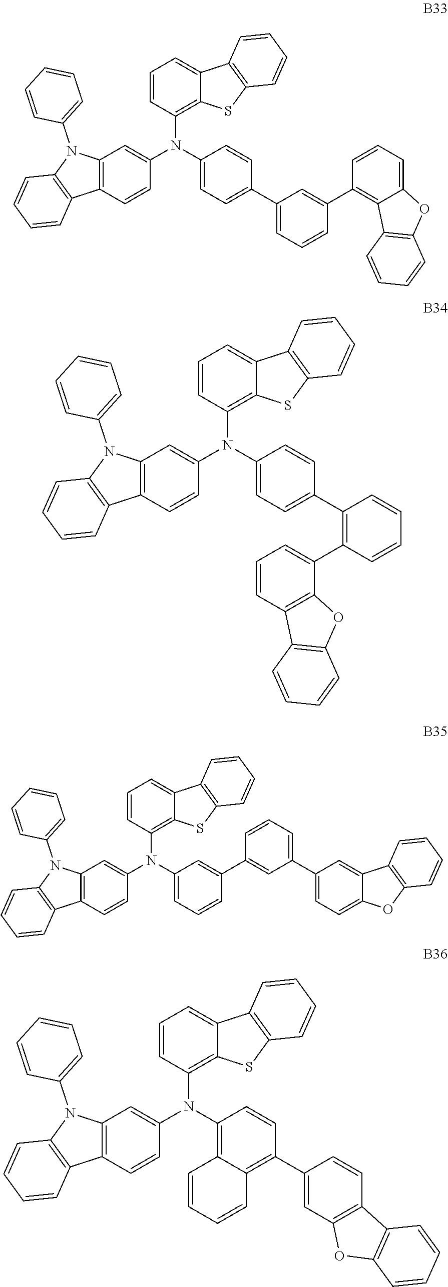 [SMILES: c1ccc(-n2c3ccccc3c3ccc(N(c4ccc(-c5cccc(-c6cccc7oc8ccccc8c67)c5)cc4)c4cccc5c4sc4ccccc45)cc32)cc1 ; c1ccc(-n2c3ccccc3c3ccc(N(c4ccc(-c5ccccc5-c5cccc6c5oc5ccccc56)cc4)c4cccc5c4sc4ccccc45)cc32)cc1 ; c1ccc(-n2c3ccccc3c3ccc(N(c4cccc(-c5cccc(-c6ccc7oc8ccccc8c7c6)c5)c4)c4cccc5c4sc4ccccc45)cc32)cc1 ; c1ccc(-n2c3ccccc3c3ccc(N(c4ccc(-c5ccc6c(c5)oc5ccccc56)c5ccccc45)c4cccc5c4sc4ccccc45)cc32)cc1]

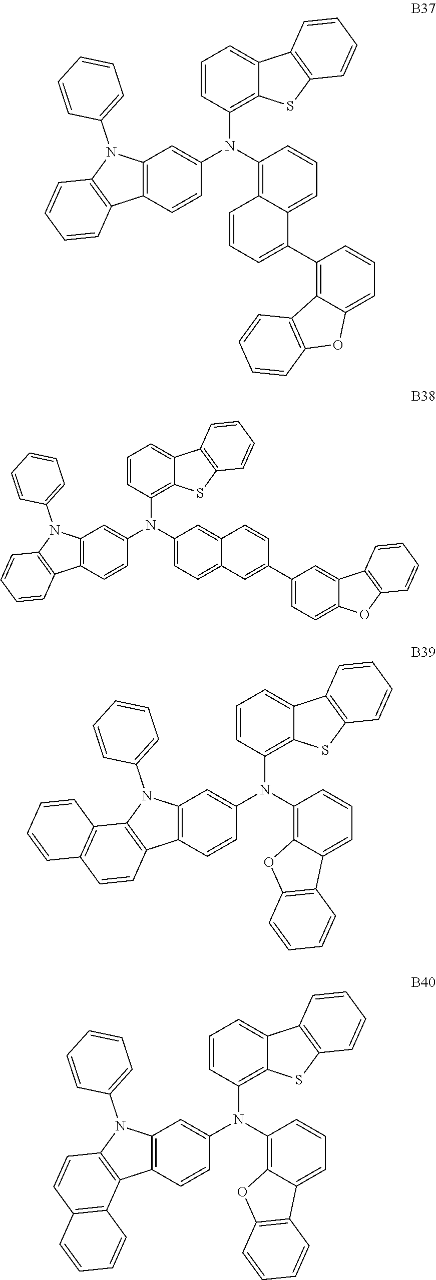 [SMILES: c1ccc(-n2c3ccccc3c3ccc(N(c4cccc5c(-c6cccc7oc8ccccc8c67)cccc45)c4cccc5c4sc4ccccc45)cc32)cc1 ; c1ccc(-n2c3ccccc3c3ccc(N(c4ccc5cc(-c6ccc7oc8ccccc8c7c6)ccc5c4)c4cccc5c4sc4ccccc45)cc32)cc1 ; c1ccc(-n2c3cc(N(c4cccc5c4oc4ccccc45)c4cccc5c4sc4ccccc45)ccc3c3ccc4ccccc4c32)cc1 ; c1ccc(-n2c3cc(N(c4cccc5c4oc4ccccc45)c4cccc5c4sc4ccccc45)ccc3c3c4ccccc4ccc32)cc1]

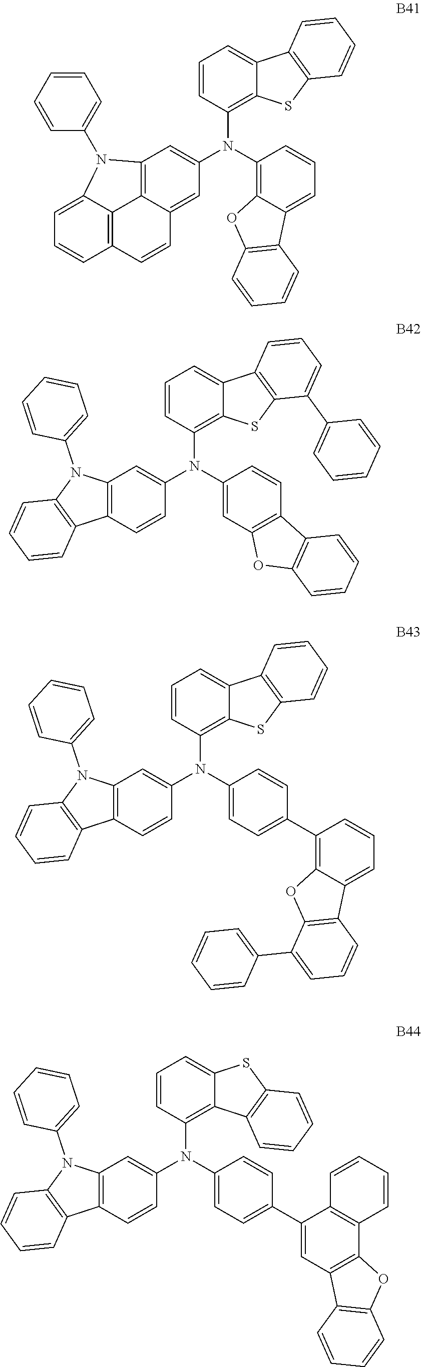

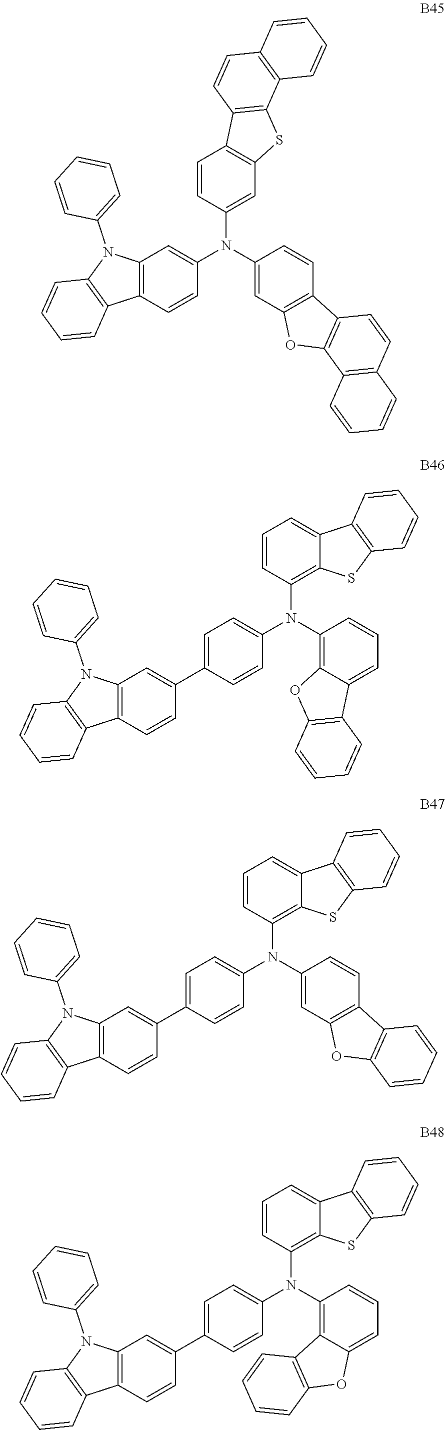 [SMILES: c1ccc(-n2c3ccccc3c3ccc(N(c4ccc5c(c4)oc4c6ccccc6ccc54)c4ccc5c(c4)sc4c6ccccc6ccc54)cc32)cc1 ; c1ccc(-n2c3ccccc3c3ccc(-c4ccc(N(c5cccc6c5oc5ccccc56)c5cccc6c5sc5ccccc56)cc4)cc32)cc1 ; c1ccc(-n2c3ccccc3c3ccc(-c4ccc(N(c5ccc6c(c5)oc5ccccc56)c5cccc6c5sc5ccccc56)cc4)cc32)cc1 ; c1ccc(-n2c3ccccc3c3ccc(-c4ccc(N(c5cccc6c5sc5ccccc56)c5cccc6oc7ccccc7c56)cc4)cc32)cc1]

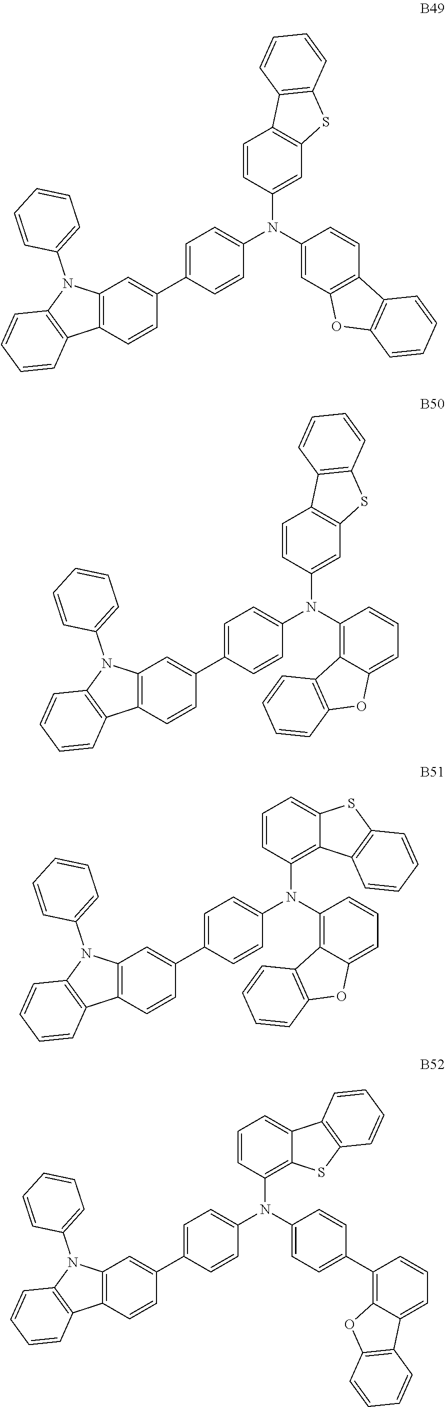 [SMILES: c1ccc(-n2c3ccccc3c3ccc(-c4ccc(N(c5ccc6c(c5)oc5ccccc56)c5ccc6c(c5)sc5ccccc56)cc4)cc32)cc1 ; c1ccc(-n2c3ccccc3c3ccc(-c4ccc(N(c5ccc6c(c5)sc5ccccc56)c5cccc6oc7ccccc7c56)cc4)cc32)cc1 ; c1ccc(-n2c3ccccc3c3ccc(-c4ccc(N(c5cccc6oc7ccccc7c56)c5cccc6sc7ccccc7c56)cc4)cc32)cc1 ; c1ccc(-n2c3ccccc3c3ccc(-c4ccc(N(c5ccc(-c6cccc7c6oc6ccccc67)cc5)c5cccc6c5sc5ccccc56)cc4)cc32)cc1]

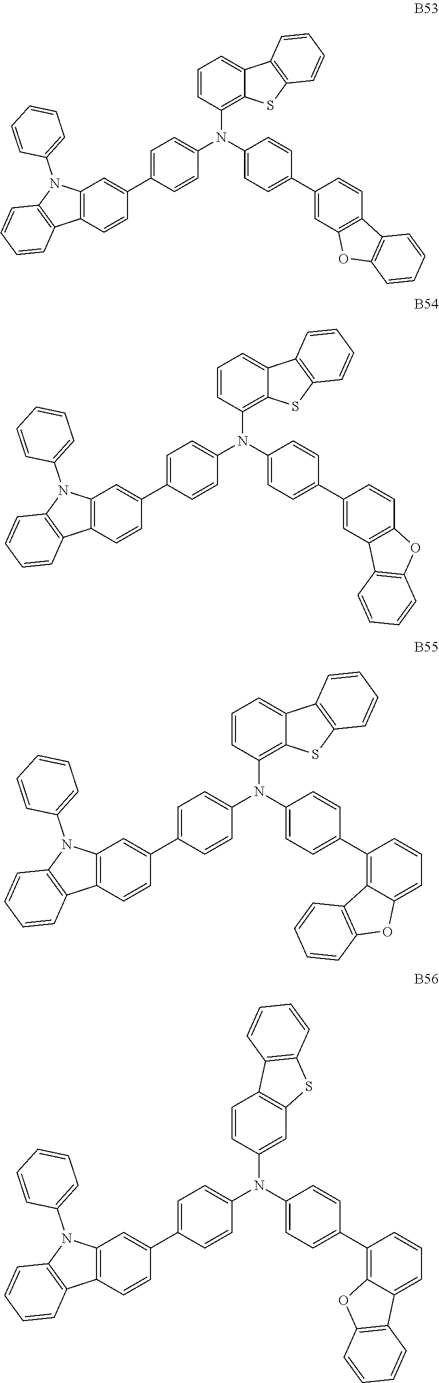 [SMILES: c1ccc(-n2c3ccccc3c3ccc(-c4ccc(N(c5ccc(-c6ccc7c(c6)oc6ccccc67)cc5)c5cccc6c5sc5ccccc56)cc4)cc32)cc1 ; c1ccc(-n2c3ccccc3c3ccc(-c4ccc(N(c5ccc(-c6ccc7oc8ccccc8c7c6)cc5)c5cccc6c5sc5ccccc56)cc4)cc32)cc1 ; c1ccc(-n2c3ccccc3c3ccc(-c4ccc(N(c5ccc(-c6cccc7oc8ccccc8c67)cc5)c5cccc6c5sc5ccccc56)cc4)cc32)cc1 ; c1ccc(-n2c3ccccc3c3ccc(-c4ccc(N(c5ccc(-c6cccc7c6oc6ccccc67)cc5)c5ccc6c(c5)sc5ccccc56)cc4)cc32)cc1]

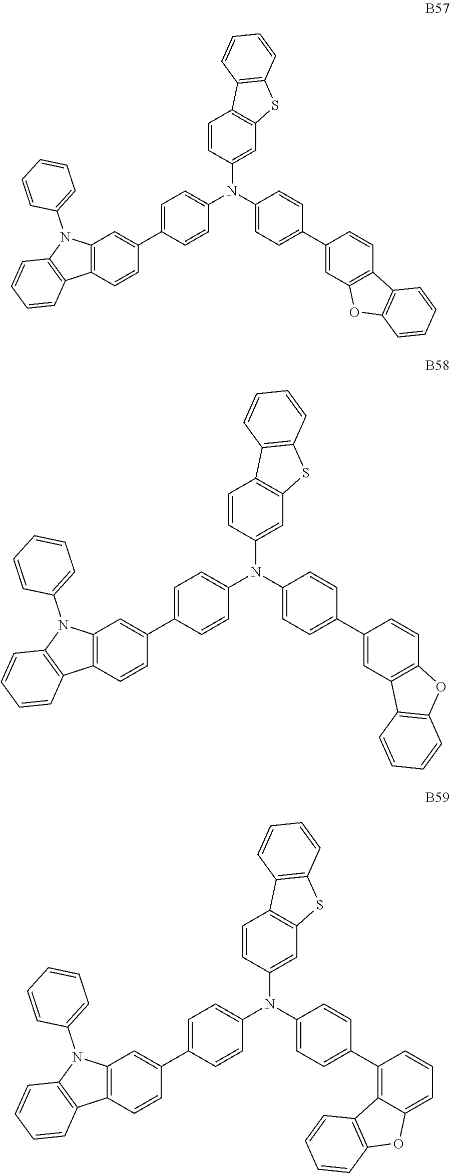 [SMILES: c1ccc(-n2c3ccccc3c3ccc(-c4ccc(N(c5ccc(-c6ccc7c(c6)oc6ccccc67)cc5)c5ccc6c(c5)sc5ccccc56)cc4)cc32)cc1 ; c1ccc(-n2c3ccccc3c3ccc(-c4ccc(N(c5ccc(-c6ccc7oc8ccccc8c7c6)cc5)c5ccc6c(c5)sc5ccccc56)cc4)cc32)cc1 ; c1ccc(-n2c3ccccc3c3ccc(-c4ccc(N(c5ccc(-c6cccc7oc8ccccc8c67)cc5)c5ccc6c(c5)sc5ccccc56)cc4)cc32)cc1]

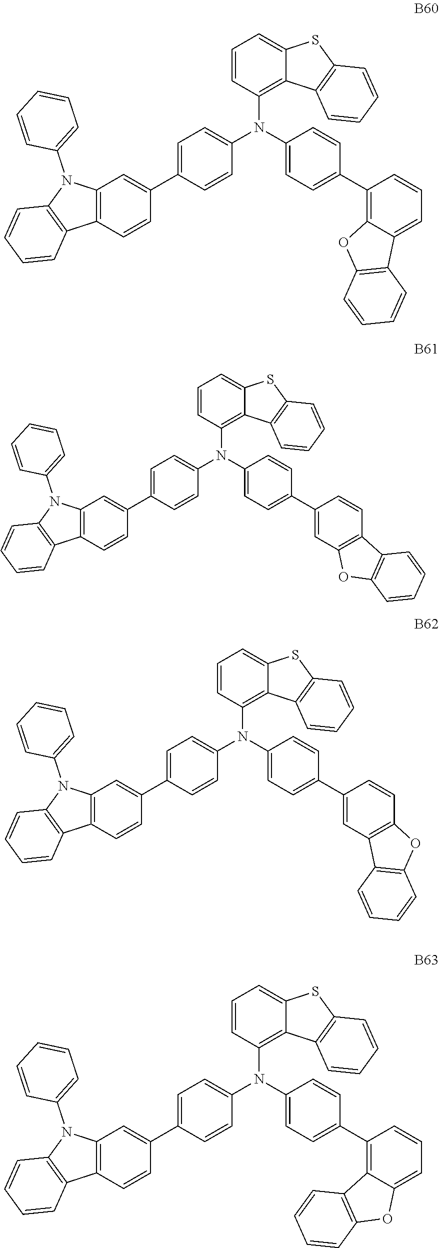 [SMILES: c1ccc(-n2c3ccccc3c3ccc(-c4ccc(N(c5ccc(-c6cccc7c6oc6ccccc67)cc5)c5cccc6sc7ccccc7c56)cc4)cc32)cc1 ; c1ccc(-n2c3ccccc3c3ccc(-c4ccc(N(c5ccc(-c6ccc7c(c6)oc6ccccc67)cc5)c5cccc6sc7ccccc7c56)cc4)cc32)cc1 ; c1ccc(-n2c3ccccc3c3ccc(-c4ccc(N(c5ccc(-c6ccc7oc8ccccc8c7c6)cc5)c5cccc6sc7ccccc7c56)cc4)cc32)cc1 ; c1ccc(-n2c3ccccc3c3ccc(-c4ccc(N(c5ccc(-c6cccc7oc8ccccc8c67)cc5)c5cccc6sc7ccccc7c56)cc4)cc32)cc1]

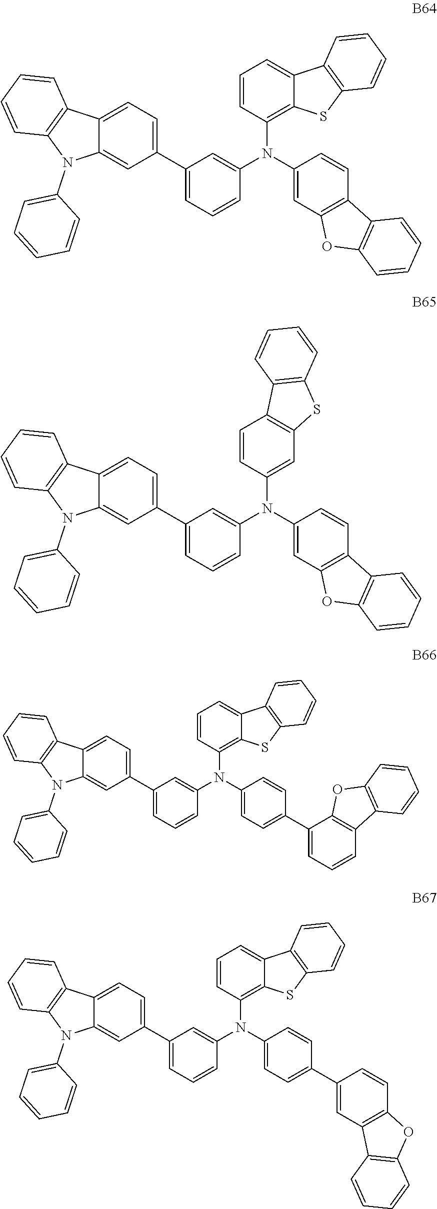 [SMILES: c1ccc(-n2c3ccccc3c3ccc(-c4cccc(N(c5ccc6c(c5)oc5ccccc56)c5cccc6c5sc5ccccc56)c4)cc32)cc1 ; c1ccc(-n2c3ccccc3c3ccc(-c4cccc(N(c5ccc6c(c5)oc5ccccc56)c5ccc6c(c5)sc5ccccc56)c4)cc32)cc1 ; c1ccc(-n2c3ccccc3c3ccc(-c4cccc(N(c5ccc(-c6cccc7c6oc6ccccc67)cc5)c5cccc6c5sc5ccccc56)c4)cc32)cc1 ; c1ccc(-n2c3ccccc3c3ccc(-c4cccc(N(c5ccc(-c6ccc7oc8ccccc8c7c6)cc5)c5cccc6c5sc5ccccc56)c4)cc32)cc1]

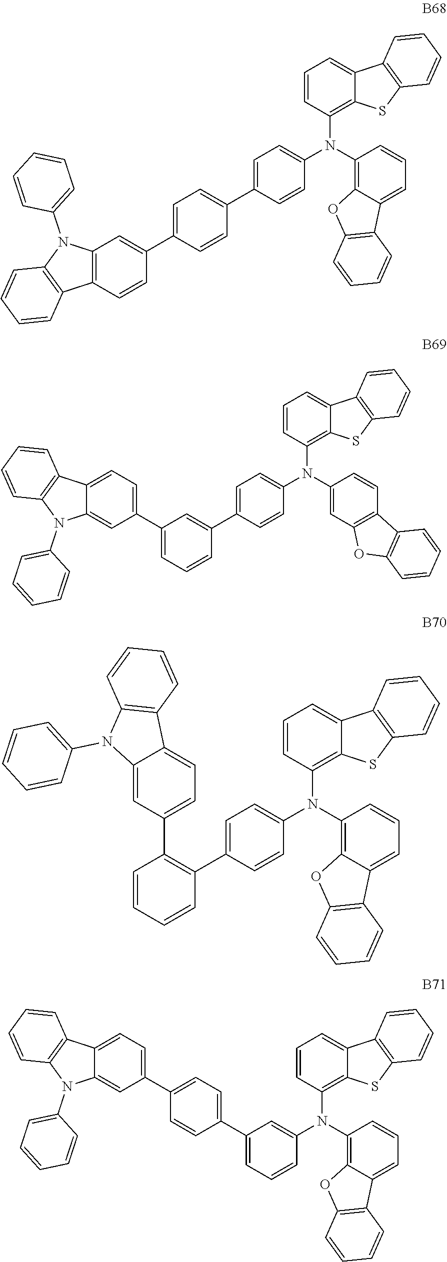 [SMILES: c1ccc(-n2c3ccccc3c3ccc(-c4ccc(-c5ccc(N(c6cccc7c6oc6ccccc67)c6cccc7c6sc6ccccc67)cc5)cc4)cc32)cc1 ; c1ccc(-n2c3ccccc3c3ccc(-c4cccc(-c5ccc(N(c6ccc7c(c6)oc6ccccc67)c6cccc7c6sc6ccccc67)cc5)c4)cc32)cc1 ; c1ccc(-n2c3ccccc3c3ccc(-c4ccccc4-c4ccc(N(c5cccc6c5oc5ccccc56)c5cccc6c5sc5ccccc56)cc4)cc32)cc1 ; c1ccc(-n2c3ccccc3c3ccc(-c4ccc(-c5cccc(N(c6cccc7c6oc6ccccc67)c6cccc7c6sc6ccccc67)c5)cc4)cc32)cc1]

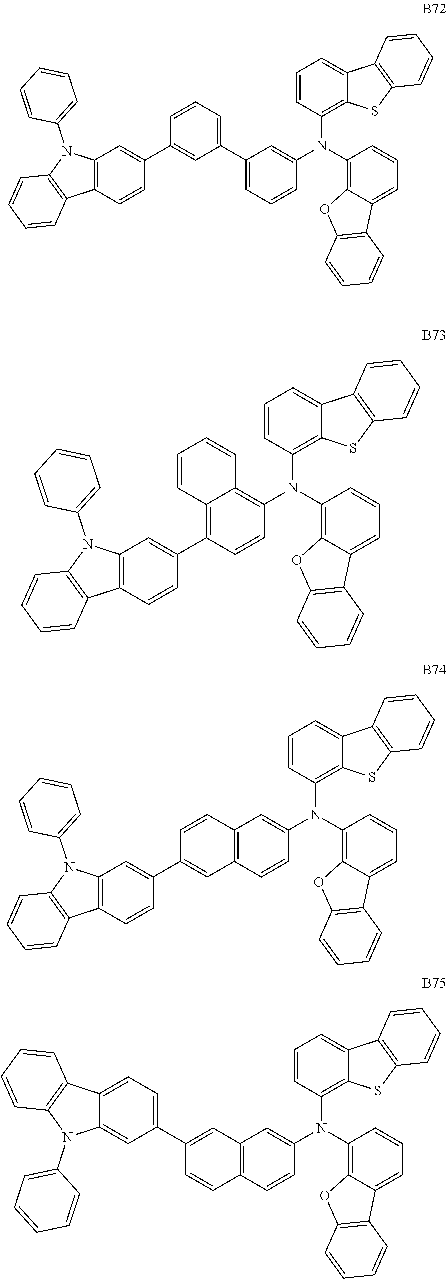 [SMILES: c1ccc(-n2c3ccccc3c3ccc(-c4cccc(-c5cccc(N(c6cccc7c6oc6ccccc67)c6cccc7c6sc6ccccc67)c5)c4)cc32)cc1 ; c1ccc(-n2c3ccccc3c3ccc(-c4ccc(N(c5cccc6c5oc5ccccc56)c5cccc6c5sc5ccccc56)c5ccccc45)cc32)cc1 ; c1ccc(-n2c3ccccc3c3ccc(-c4ccc5cc(N(c6cccc7c6oc6ccccc67)c6cccc7c6sc6ccccc67)ccc5c4)cc32)cc1 ; c1ccc(-n2c3ccccc3c3ccc(-c4ccc5ccc(N(c6cccc7c6oc6ccccc67)c6cccc7c6sc6ccccc67)cc5c4)cc32)cc1]

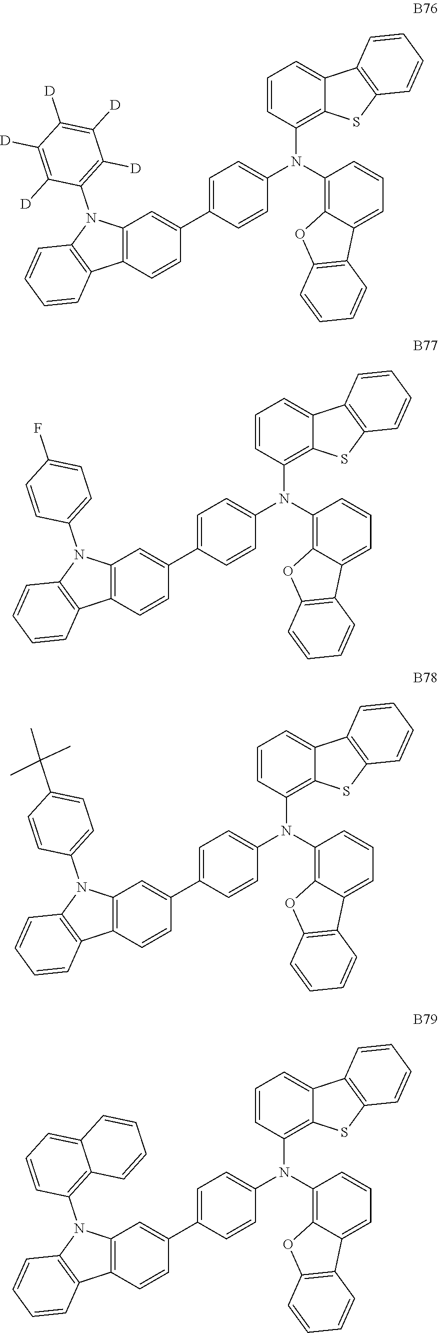 [SMILES: [2H]c1c([2H])c([2H])c(-n2c3ccccc3c3ccc(-c4ccc(N(c5cccc6c5oc5ccccc56)c5cccc6c5sc5ccccc56)cc4)cc32)c([2H])c1[2H] ; Fc1ccc(-n2c3ccccc3c3ccc(-c4ccc(N(c5cccc6c5oc5ccccc56)c5cccc6c5sc5ccccc56)cc4)cc32)cc1 ; CC(C)(C)c1ccc(-n2c3ccccc3c3ccc(-c4ccc(N(c5cccc6c5oc5ccccc56)c5cccc6c5sc5ccccc56)cc4)cc32)cc1 ; c1ccc2c(-n3c4ccccc4c4ccc(-c5ccc(N(c6cccc7c6oc6ccccc67)c6cccc7c6sc6ccccc67)cc5)cc43)cccc2c1]

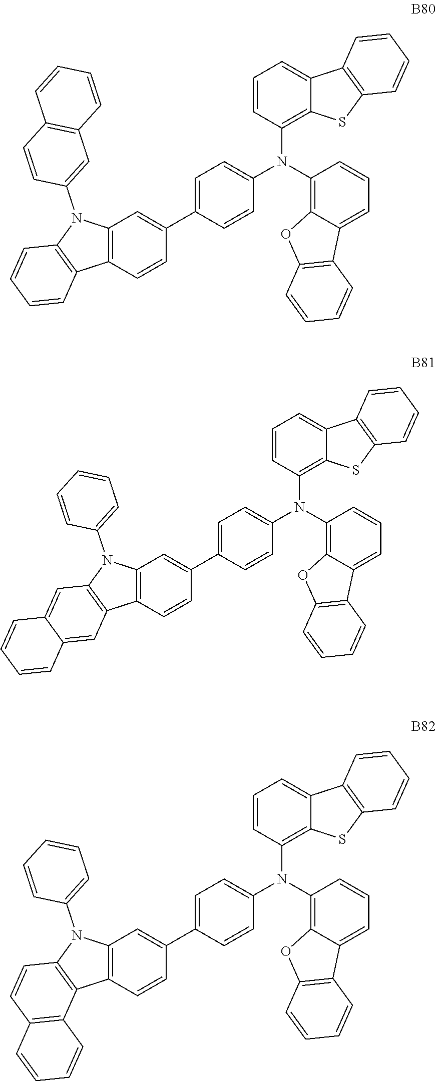 [SMILES: c1ccc2cc(-n3c4ccccc4c4ccc(-c5ccc(N(c6cccc7c6oc6ccccc67)c6cccc7c6sc6ccccc67)cc5)cc43)ccc2c1 ; c1ccc(-n2c3cc(-c4ccc(N(c5cccc6c5oc5ccccc56)c5cccc6c5sc5ccccc56)cc4)ccc3c3cc4ccccc4cc32)cc1 ; c1ccc(-n2c3cc(-c4ccc(N(c5cccc6c5oc5ccccc56)c5cccc6c5sc5ccccc56)cc4)ccc3c3c4ccccc4ccc32)cc1]

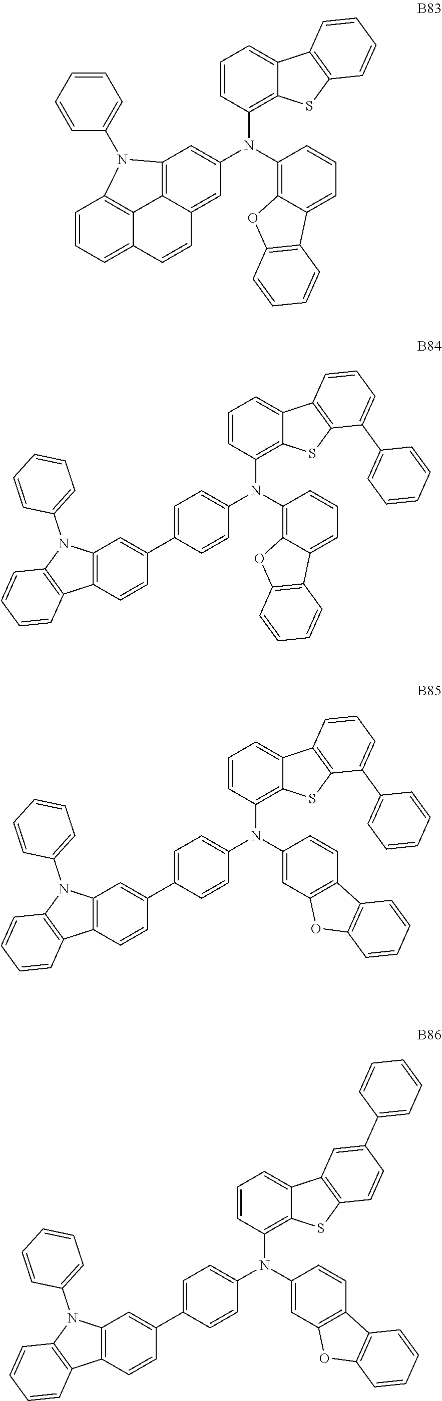 [SMILES: c1ccc(-n2c3cccc4ccc5cc(N(c6cccc7c6oc6ccccc67)c6cccc7c6sc6ccccc67)cc2c5c43)cc1 ; c1ccc(-c2cccc3c2sc2c(N(c4ccc(-c5ccc6c7ccccc7n(-c7ccccc7)c6c5)cc4)c4cccc5c4oc4ccccc45)cccc23)cc1 ; c1ccc(-c2cccc3c2sc2c(N(c4ccc(-c5ccc6c7ccccc7n(-c7ccccc7)c6c5)cc4)c4ccc5c(c4)oc4ccccc45)cccc23)cc1 ; c1ccc(-c2ccc3sc4c(N(c5ccc(-c6ccc7c8ccccc8n(-c8ccccc8)c7c6)cc5)c5ccc6c(c5)oc5ccccc56)cccc4c3c2)cc1]

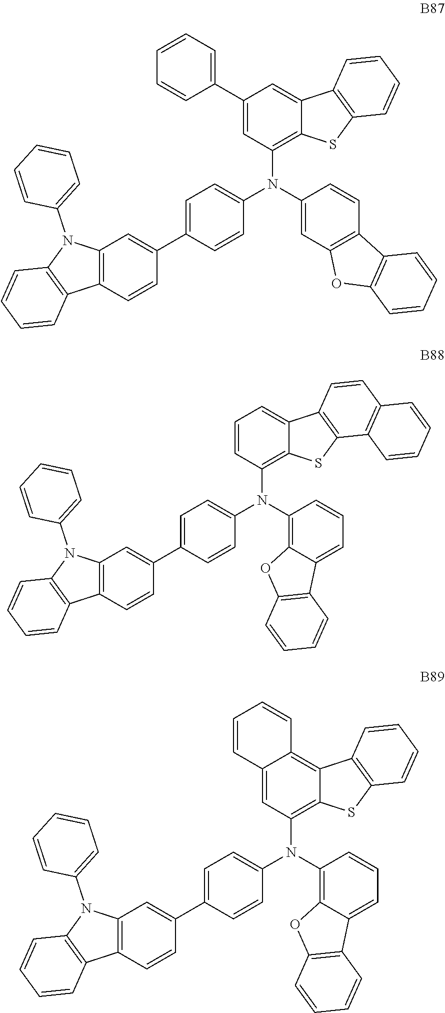 [SMILES: c1ccc(-c2cc(N(c3ccc(-c4ccc5c6ccccc6n(-c6ccccc6)c5c4)cc3)c3ccc4c(c3)oc3ccccc34)c3sc4ccccc4c3c2)cc1 ; c1ccc(-n2c3ccccc3c3ccc(-c4ccc(N(c5cccc6c5oc5ccccc56)c5cccc6c5sc5c7ccccc7ccc65)cc4)cc32)cc1 ; c1ccc(-n2c3ccccc3c3ccc(-c4ccc(N(c5cccc6c5oc5ccccc56)c5cc6ccccc6c6c5sc5ccccc56)cc4)cc32)cc1]

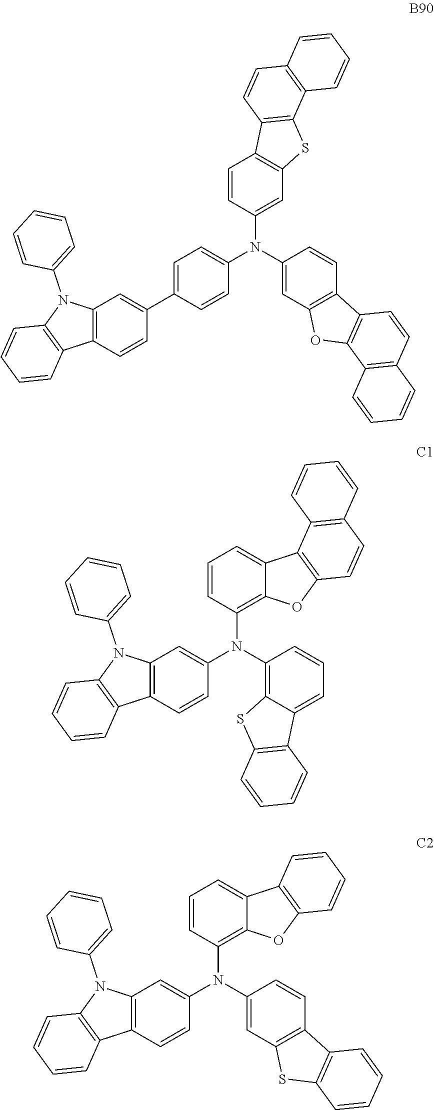 [SMILES: c1ccc(-n2c3ccccc3c3ccc(-c4ccc(N(c5ccc6c(c5)oc5c7ccccc7ccc65)c5ccc6c(c5)sc5c7ccccc7ccc65)cc4)cc32)cc1 ; c1ccc(-n2c3ccccc3c3ccc(N(c4cccc5c4oc4ccc6ccccc6c45)c4cccc5c4sc4ccccc45)cc32)cc1 ; c1ccc(-n2c3ccccc3c3ccc(N(c4ccc5c(c4)sc4ccccc45)c4cccc5c4oc4ccccc45)cc32)cc1]

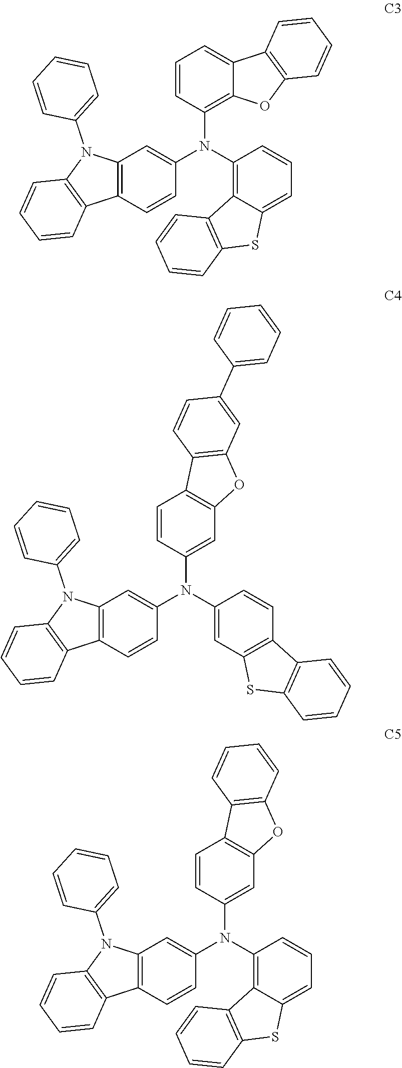

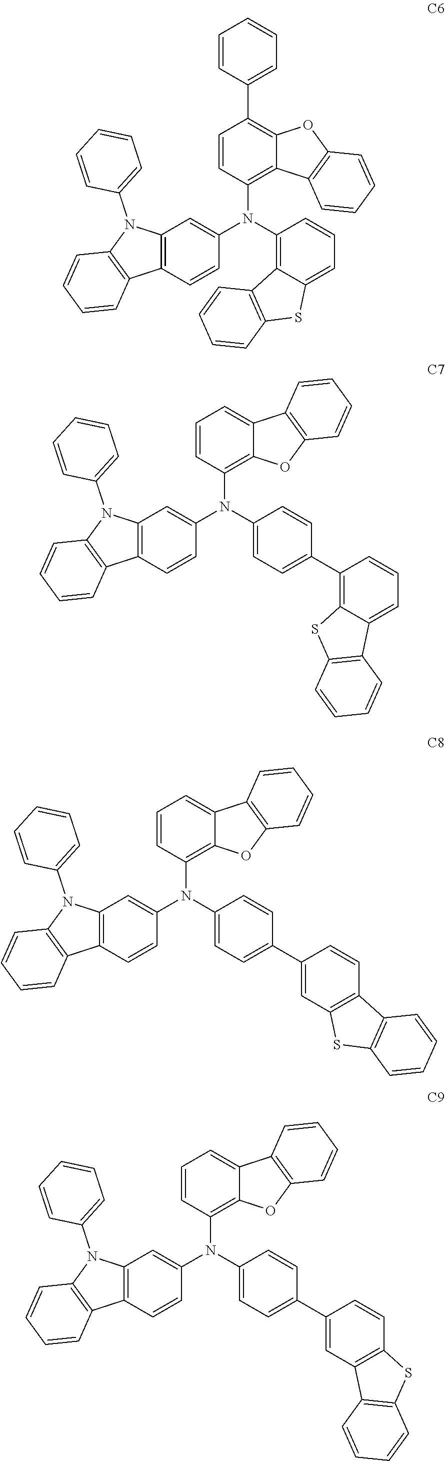 [SMILES: c1ccc(-c2ccc(N(c3ccc4c5ccccc5n(-c5ccccc5)c4c3)c3cccc4sc5ccccc5c34)c3c2oc2ccccc23)cc1 ; c1ccc(-n2c3ccccc3c3ccc(N(c4ccc(-c5cccc6c5sc5ccccc56)cc4)c4cccc5c4oc4ccccc45)cc32)cc1 ; c1ccc(-n2c3ccccc3c3ccc(N(c4ccc(-c5ccc6c(c5)sc5ccccc56)cc4)c4cccc5c4oc4ccccc45)cc32)cc1 ; c1ccc(-n2c3ccccc3c3ccc(N(c4ccc(-c5ccc6sc7ccccc7c6c5)cc4)c4cccc5c4oc4ccccc45)cc32)cc1]

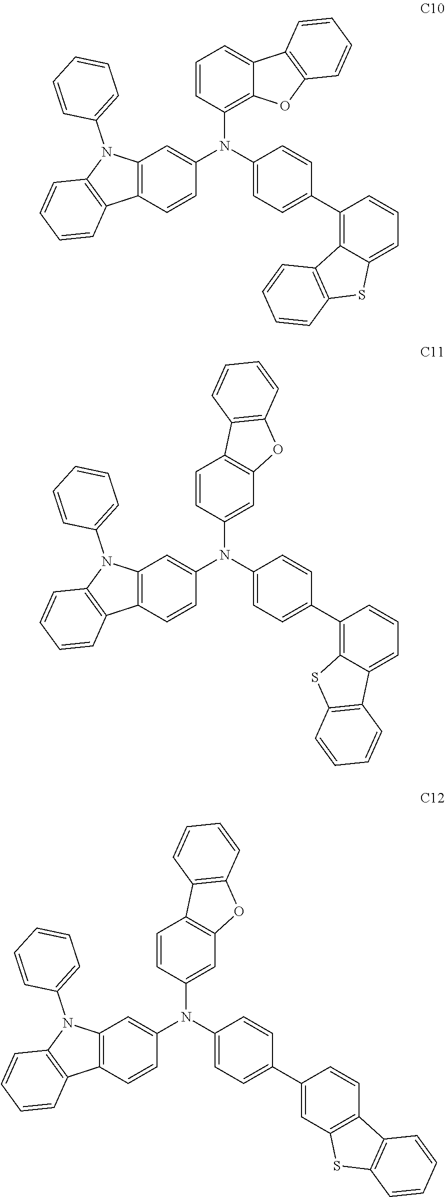

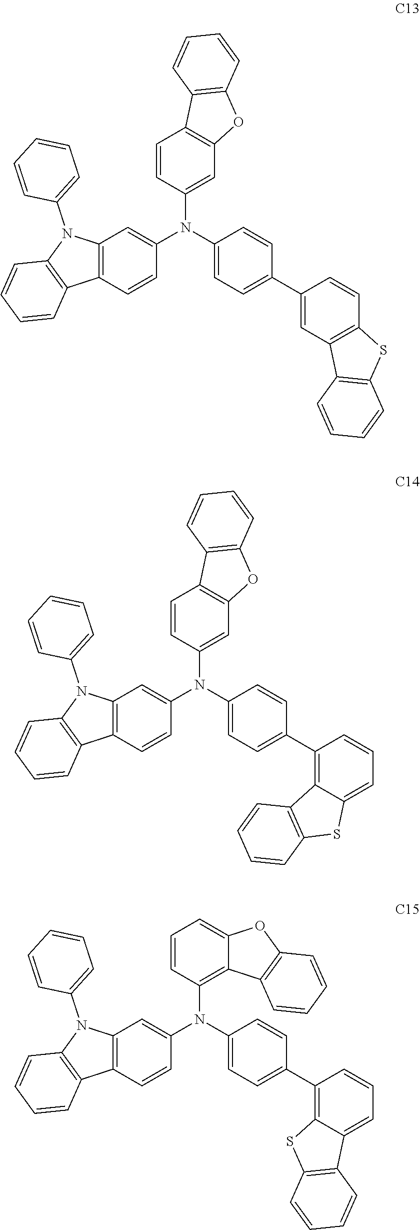 [SMILES: c1ccc(-n2c3ccccc3c3ccc(N(c4ccc(-c5ccc6sc7ccccc7c6c5)cc4)c4ccc5c(c4)oc4ccccc45)cc32)cc1 ; c1ccc(-n2c3ccccc3c3ccc(N(c4ccc(-c5cccc6sc7ccccc7c56)cc4)c4ccc5c(c4)oc4ccccc45)cc32)cc1 ; c1ccc(-n2c3ccccc3c3ccc(N(c4ccc(-c5cccc6c5sc5ccccc56)cc4)c4cccc5oc6ccccc6c45)cc32)cc1]

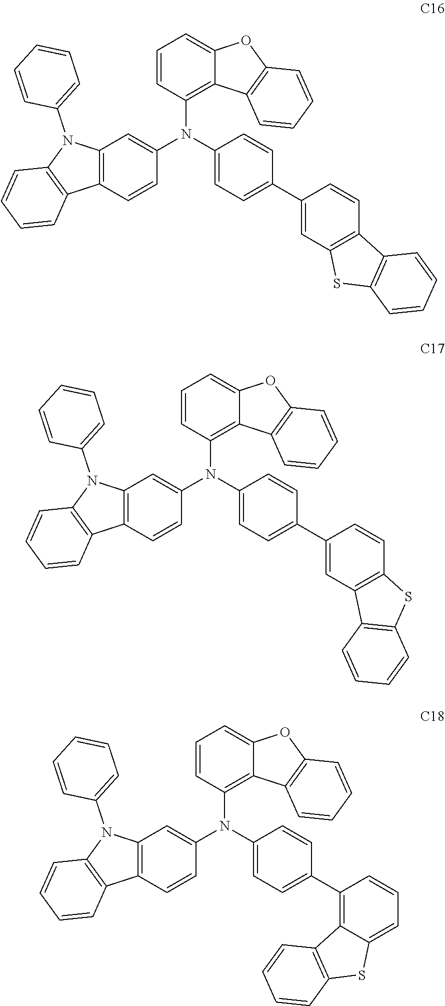 [SMILES: c1ccc(-n2c3ccccc3c3ccc(N(c4ccc(-c5ccc6c(c5)sc5ccccc56)cc4)c4cccc5oc6ccccc6c45)cc32)cc1 ; c1ccc(-n2c3ccccc3c3ccc(N(c4ccc(-c5ccc6sc7ccccc7c6c5)cc4)c4cccc5oc6ccccc6c45)cc32)cc1 ; c1ccc(-n2c3ccccc3c3ccc(N(c4ccc(-c5cccc6sc7ccccc7c56)cc4)c4cccc5oc6ccccc6c45)cc32)cc1]

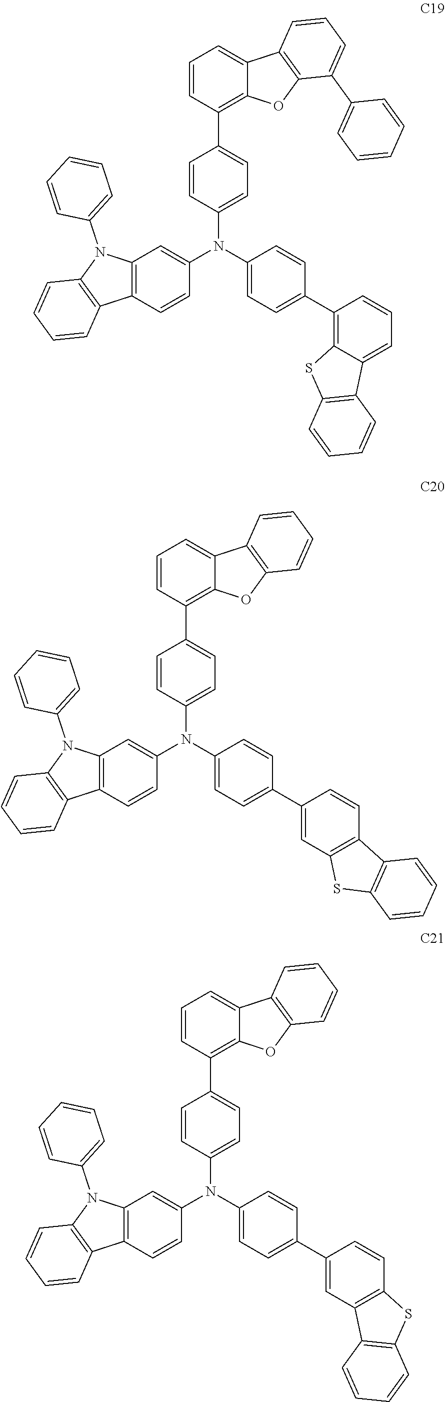 [SMILES: c1ccc(-c2cccc3c2oc2c(-c4ccc(N(c5ccc(-c6cccc7c6sc6ccccc67)cc5)c5ccc6c7ccccc7n(-c7ccccc7)c6c5)cc4)cccc23)cc1 ; c1ccc(-n2c3ccccc3c3ccc(N(c4ccc(-c5ccc6c(c5)sc5ccccc56)cc4)c4ccc(-c5cccc6c5oc5ccccc56)cc4)cc32)cc1 ; c1ccc(-n2c3ccccc3c3ccc(N(c4ccc(-c5ccc6sc7ccccc7c6c5)cc4)c4ccc(-c5cccc6c5oc5ccccc56)cc4)cc32)cc1]

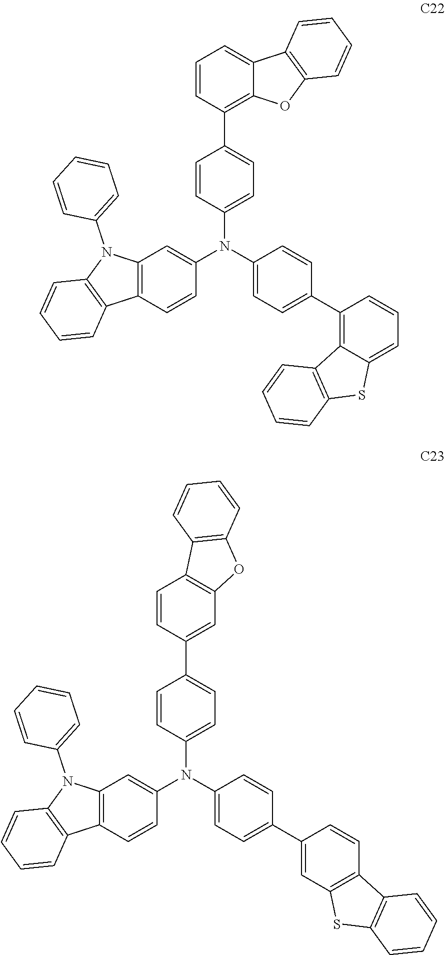 [SMILES: c1ccc(-n2c3ccccc3c3ccc(N(c4ccc(-c5cccc6c5oc5ccccc56)cc4)c4ccc(-c5cccc6sc7ccccc7c56)cc4)cc32)cc1 ; c1ccc(-n2c3ccccc3c3ccc(N(c4ccc(-c5ccc6c(c5)oc5ccccc56)cc4)c4ccc(-c5ccc6c(c5)sc5ccccc56)cc4)cc32)cc1]

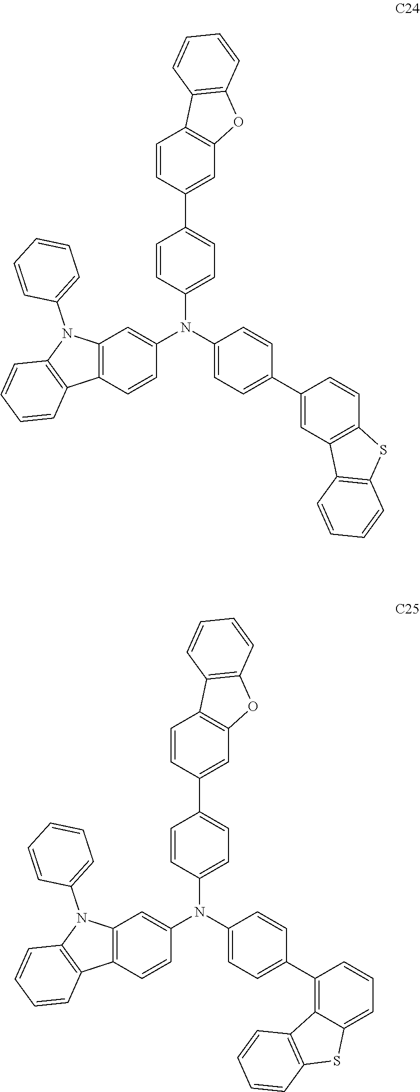 [SMILES: c1ccc(-n2c3ccccc3c3ccc(N(c4ccc(-c5ccc6c(c5)oc5ccccc56)cc4)c4ccc(-c5ccc6sc7ccccc7c6c5)cc4)cc32)cc1 ; c1ccc(-n2c3ccccc3c3ccc(N(c4ccc(-c5ccc6c(c5)oc5ccccc56)cc4)c4ccc(-c5cccc6sc7ccccc7c56)cc4)cc32)cc1]

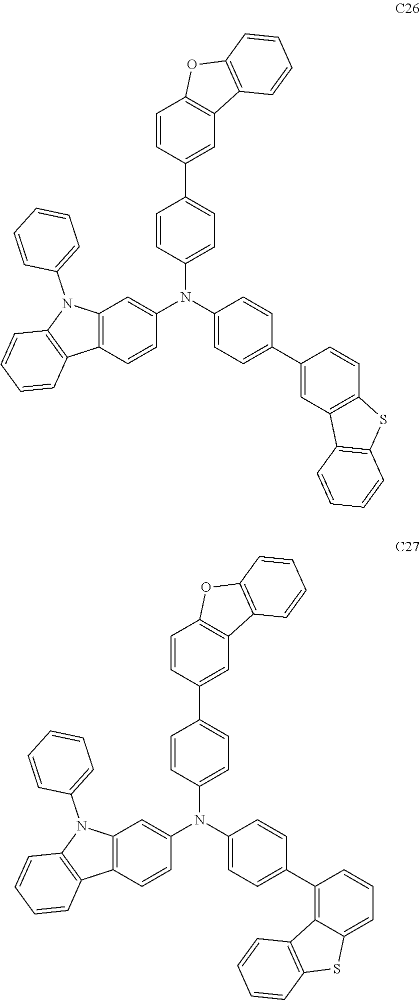 [SMILES: c1ccc(-n2c3ccccc3c3ccc(N(c4ccc(-c5ccc6oc7ccccc7c6c5)cc4)c4ccc(-c5ccc6sc7ccccc7c6c5)cc4)cc32)cc1 ; c1ccc(-n2c3ccccc3c3ccc(N(c4ccc(-c5ccc6oc7ccccc7c6c5)cc4)c4ccc(-c5cccc6sc7ccccc7c56)cc4)cc32)cc1]

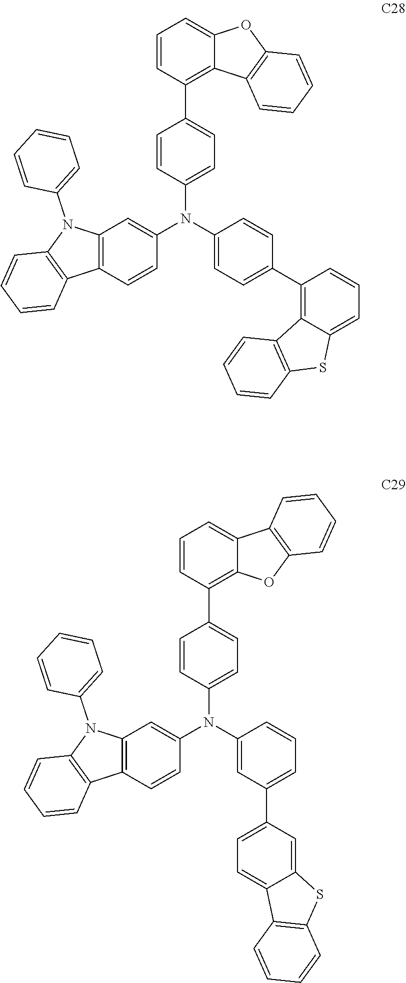 [SMILES: c1ccc(-n2c3ccccc3c3ccc(N(c4ccc(-c5cccc6oc7ccccc7c56)cc4)c4ccc(-c5cccc6sc7ccccc7c56)cc4)cc32)cc1 ; c1ccc(-n2c3ccccc3c3ccc(N(c4ccc(-c5cccc6c5oc5ccccc56)cc4)c4cccc(-c5ccc6c(c5)sc5ccccc56)c4)cc32)cc1]

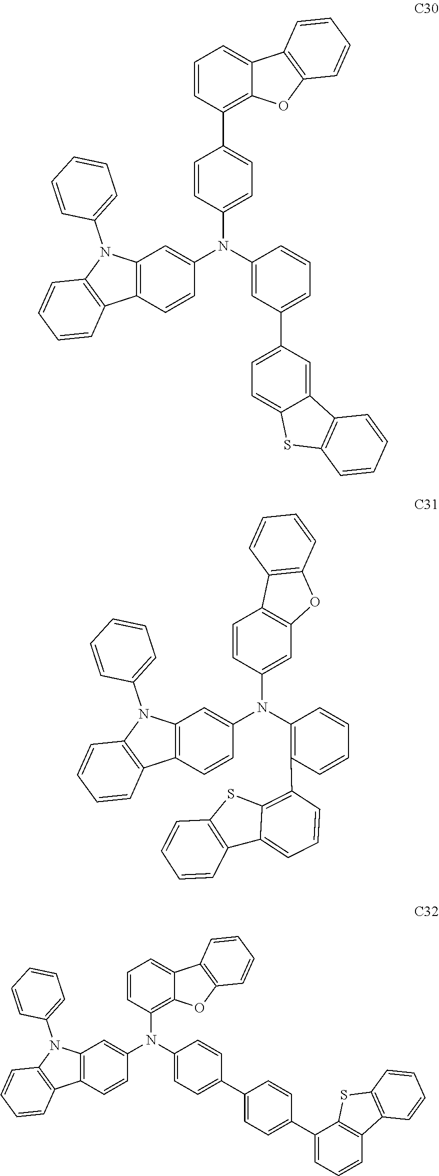 [SMILES: c1ccc(-n2c3ccccc3c3ccc(N(c4ccc(-c5cccc6c5oc5ccccc56)cc4)c4cccc(-c5ccc6sc7ccccc7c6c5)c4)cc32)cc1 ; c1ccc(-n2c3ccccc3c3ccc(N(c4ccc5c(c4)oc4ccccc45)c4ccccc4-c4cccc5c4sc4ccccc45)cc32)cc1 ; c1ccc(-n2c3ccccc3c3ccc(N(c4ccc(-c5ccc(-c6cccc7c6sc6ccccc67)cc5)cc4)c4cccc5c4oc4ccccc45)cc32)cc1]

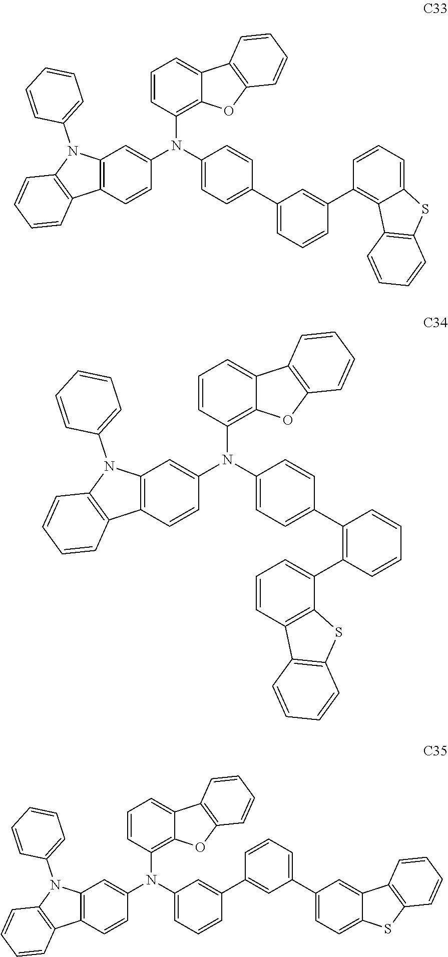 [SMILES: c1ccc(-n2c3ccccc3c3ccc(N(c4ccc(-c5cccc(-c6cccc7sc8ccccc8c67)c5)cc4)c4cccc5c4oc4ccccc45)cc32)cc1 ; c1ccc(-n2c3ccccc3c3ccc(N(c4ccc(-c5ccccc5-c5cccc6c5sc5ccccc56)cc4)c4cccc5c4oc4ccccc45)cc32)cc1 ; c1ccc(-n2c3ccccc3c3ccc(N(c4cccc(-c5cccc(-c6ccc7sc8ccccc8c7c6)c5)c4)c4cccc5c4oc4ccccc45)cc32)cc1]

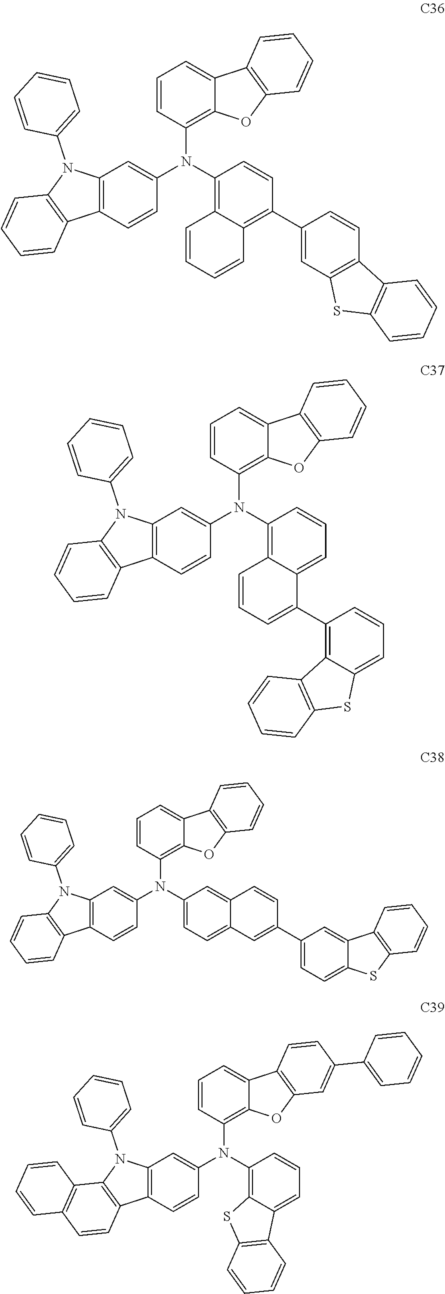 [SMILES: c1ccc(-n2c3ccccc3c3ccc(N(c4ccc(-c5ccc6c(c5)sc5ccccc56)c5ccccc45)c4cccc5c4oc4ccccc45)cc32)cc1 ; c1ccc(-n2c3ccccc3c3ccc(N(c4cccc5c(-c6cccc7sc8ccccc8c67)cccc45)c4cccc5c4oc4ccccc45)cc32)cc1 ; c1ccc(-n2c3ccccc3c3ccc(N(c4ccc5cc(-c6ccc7sc8ccccc8c7c6)ccc5c4)c4cccc5c4oc4ccccc45)cc32)cc1 ; c1ccc(-c2ccc3c(c2)oc2c(N(c4ccc5c6ccc7ccccc7c6n(-c6ccccc6)c5c4)c4cccc5c4sc4ccccc45)cccc23)cc1]

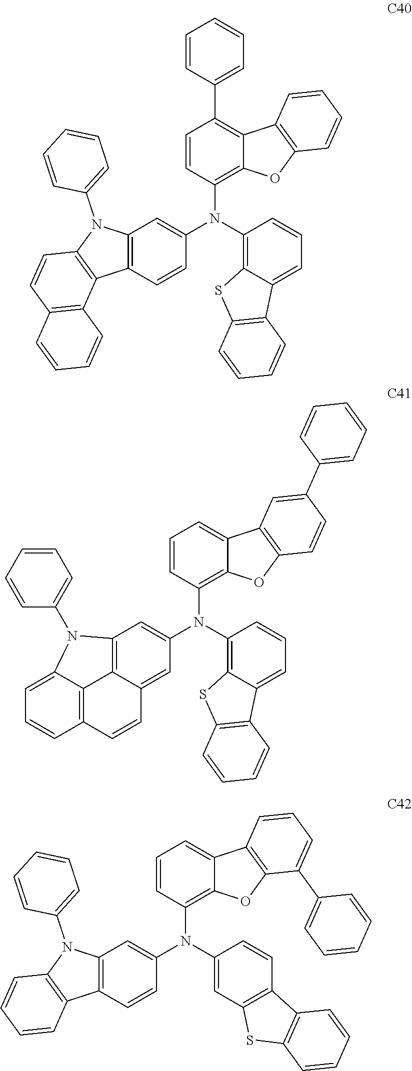

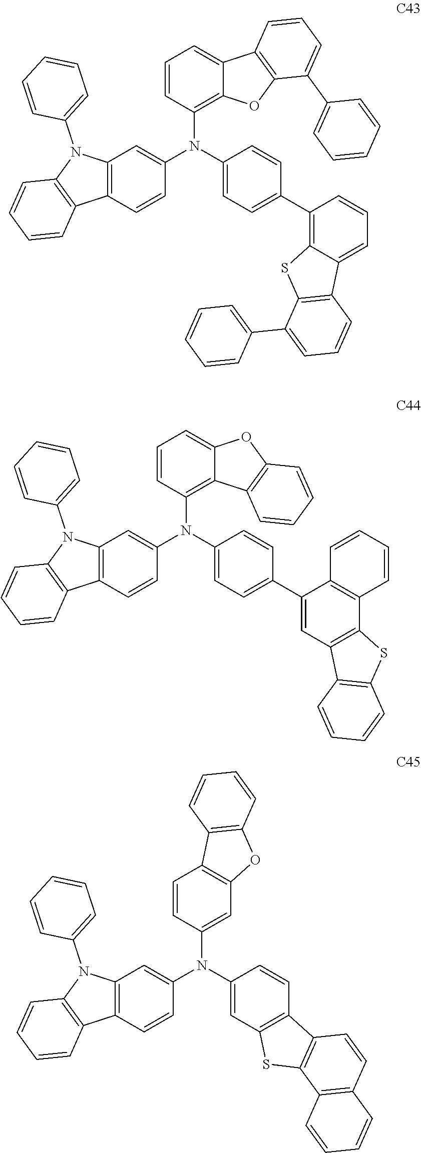 [SMILES: c1ccc(-c2cccc3c2oc2c(N(c4ccc(-c5cccc6c5sc5c(-c7ccccc7)cccc56)cc4)c4ccc5c6ccccc6n(-c6ccccc6)c5c4)cccc23)cc1 ; c1ccc(-n2c3ccccc3c3ccc(N(c4ccc(-c5cc6c7ccccc7sc6c6ccccc56)cc4)c4cccc5oc6ccccc6c45)cc32)cc1 ; c1ccc(-n2c3ccccc3c3ccc(N(c4ccc5c(c4)oc4ccccc45)c4ccc5c(c4)sc4c6ccccc6ccc54)cc32)cc1]

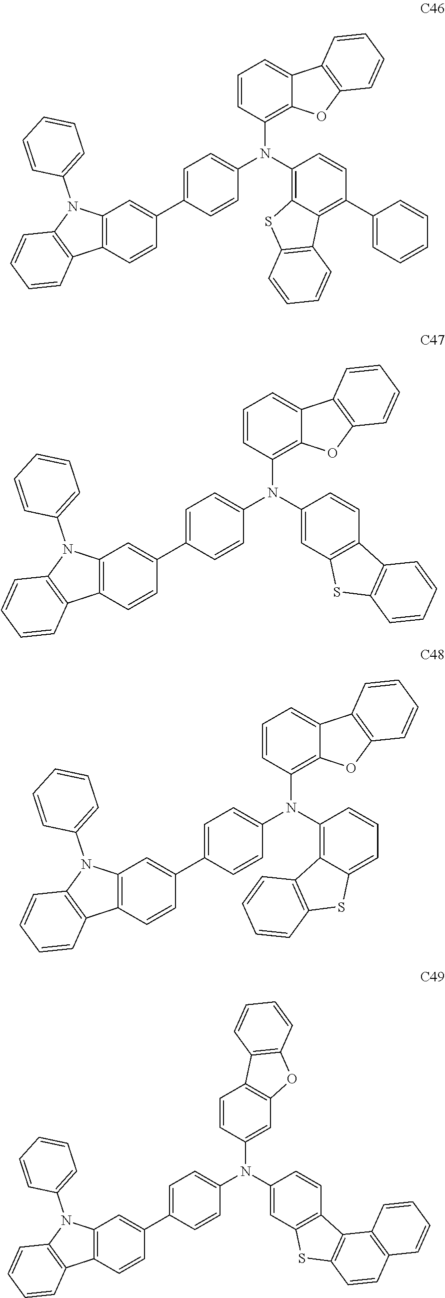 [SMILES: c1ccc(-c2ccc(N(c3ccc(-c4ccc5c6ccccc6n(-c6ccccc6)c5c4)cc3)c3cccc4c3oc3ccccc34)c3sc4ccccc4c23)cc1 ; c1ccc(-n2c3ccccc3c3ccc(-c4ccc(N(c5ccc6c(c5)sc5ccccc56)c5cccc6c5oc5ccccc56)cc4)cc32)cc1 ; c1ccc(-n2c3ccccc3c3ccc(-c4ccc(N(c5cccc6c5oc5ccccc56)c5cccc6sc7ccccc7c56)cc4)cc32)cc1 ; c1ccc(-n2c3ccccc3c3ccc(-c4ccc(N(c5ccc6c(c5)oc5ccccc56)c5ccc6c(c5)sc5ccc7ccccc7c56)cc4)cc32)cc1]

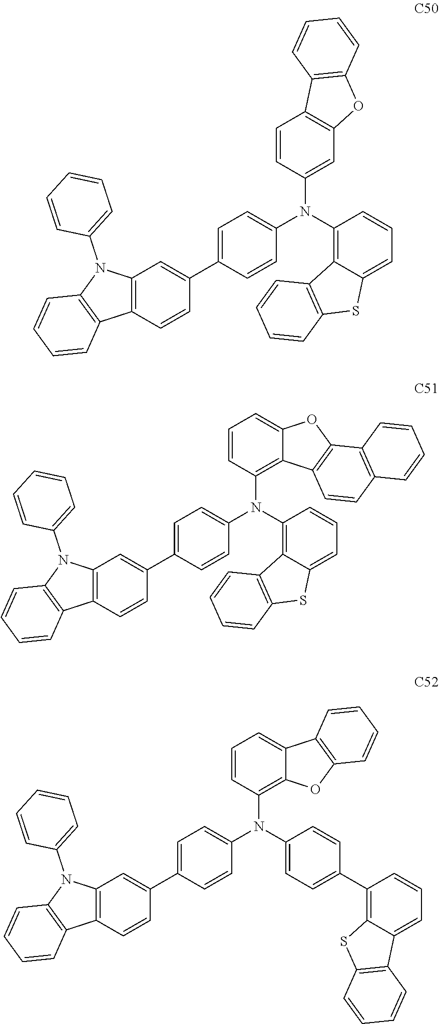 [SMILES: c1ccc(-n2c3ccccc3c3ccc(-c4ccc(N(c5ccc6c(c5)oc5ccccc56)c5cccc6sc7ccccc7c56)cc4)cc32)cc1 ; c1ccc(-n2c3ccccc3c3ccc(-c4ccc(N(c5cccc6oc7c8ccccc8ccc7c56)c5cccc6sc7ccccc7c56)cc4)cc32)cc1 ; c1ccc(-n2c3ccccc3c3ccc(-c4ccc(N(c5ccc(-c6cccc7c6sc6ccccc67)cc5)c5cccc6c5oc5ccccc56)cc4)cc32)cc1]

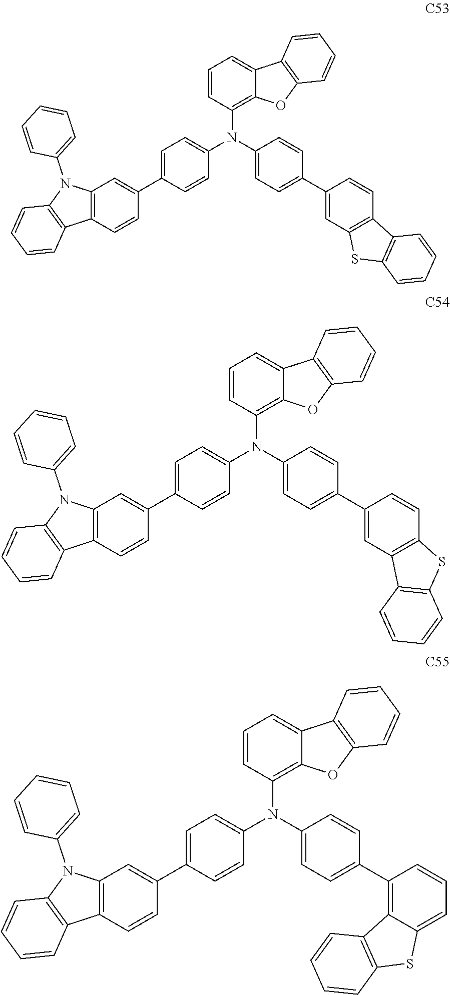 [SMILES: c1ccc(-n2c3ccccc3c3ccc(-c4ccc(N(c5ccc(-c6ccc7c(c6)sc6ccccc67)cc5)c5cccc6c5oc5ccccc56)cc4)cc32)cc1 ; c1ccc(-n2c3ccccc3c3ccc(-c4ccc(N(c5ccc(-c6ccc7sc8ccccc8c7c6)cc5)c5cccc6c5oc5ccccc56)cc4)cc32)cc1 ; c1ccc(-n2c3ccccc3c3ccc(-c4ccc(N(c5ccc(-c6cccc7sc8ccccc8c67)cc5)c5cccc6c5oc5ccccc56)cc4)cc32)cc1]

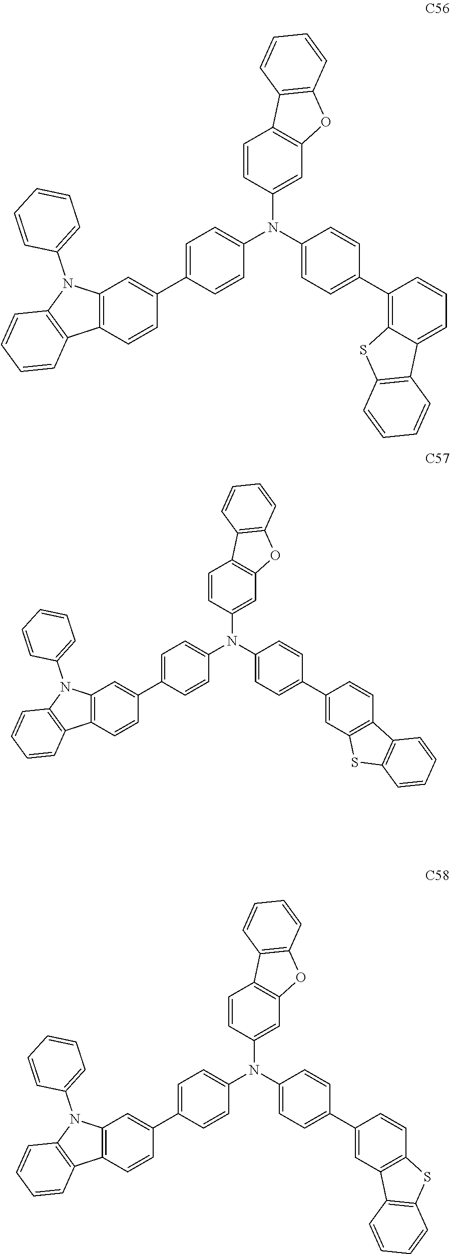 [SMILES: c1ccc(-n2c3ccccc3c3ccc(-c4ccc(N(c5ccc(-c6cccc7c6sc6ccccc67)cc5)c5ccc6c(c5)oc5ccccc56)cc4)cc32)cc1 ; c1ccc(-n2c3ccccc3c3ccc(-c4ccc(N(c5ccc(-c6ccc7c(c6)sc6ccccc67)cc5)c5ccc6c(c5)oc5ccccc56)cc4)cc32)cc1 ; c1ccc(-n2c3ccccc3c3ccc(-c4ccc(N(c5ccc(-c6ccc7sc8ccccc8c7c6)cc5)c5ccc6c(c5)oc5ccccc56)cc4)cc32)cc1]

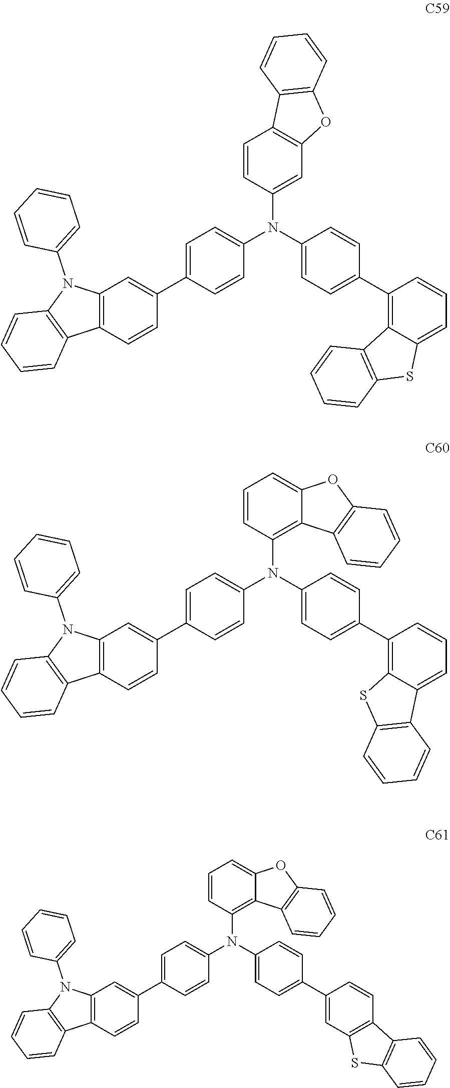 [SMILES: c1ccc(-n2c3ccccc3c3ccc(-c4ccc(N(c5ccc(-c6cccc7sc8ccccc8c67)cc5)c5ccc6c(c5)oc5ccccc56)cc4)cc32)cc1 ; c1ccc(-n2c3ccccc3c3ccc(-c4ccc(N(c5ccc(-c6cccc7c6sc6ccccc67)cc5)c5cccc6oc7ccccc7c56)cc4)cc32)cc1 ; c1ccc(-n2c3ccccc3c3ccc(-c4ccc(N(c5ccc(-c6ccc7c(c6)sc6ccccc67)cc5)c5cccc6oc7ccccc7c56)cc4)cc32)cc1]

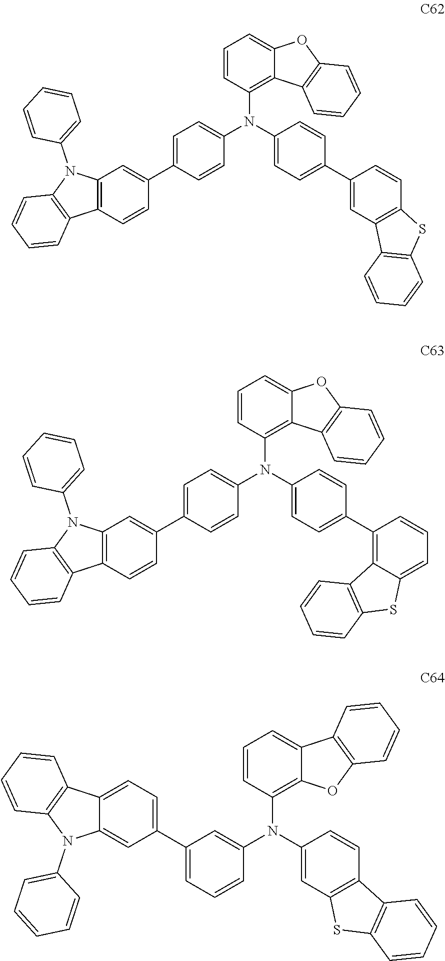 [SMILES: c1ccc(-n2c3ccccc3c3ccc(-c4ccc(N(c5ccc(-c6ccc7sc8ccccc8c7c6)cc5)c5cccc6oc7ccccc7c56)cc4)cc32)cc1 ; c1ccc(-n2c3ccccc3c3ccc(-c4ccc(N(c5ccc(-c6cccc7sc8ccccc8c67)cc5)c5cccc6oc7ccccc7c56)cc4)cc32)cc1 ; c1ccc(-n2c3ccccc3c3ccc(-c4cccc(N(c5ccc6c(c5)sc5ccccc56)c5cccc6c5oc5ccccc56)c4)cc32)cc1]

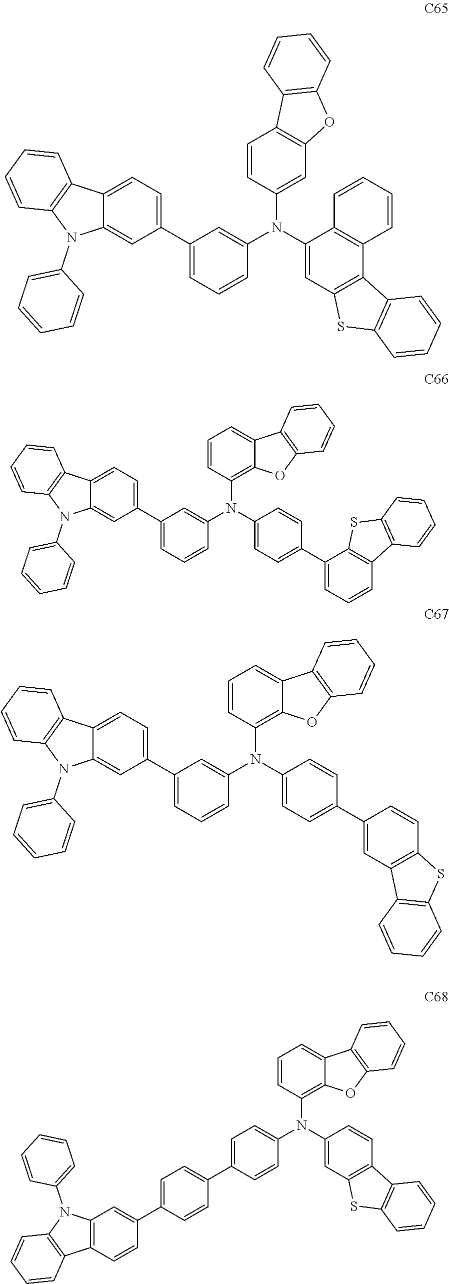

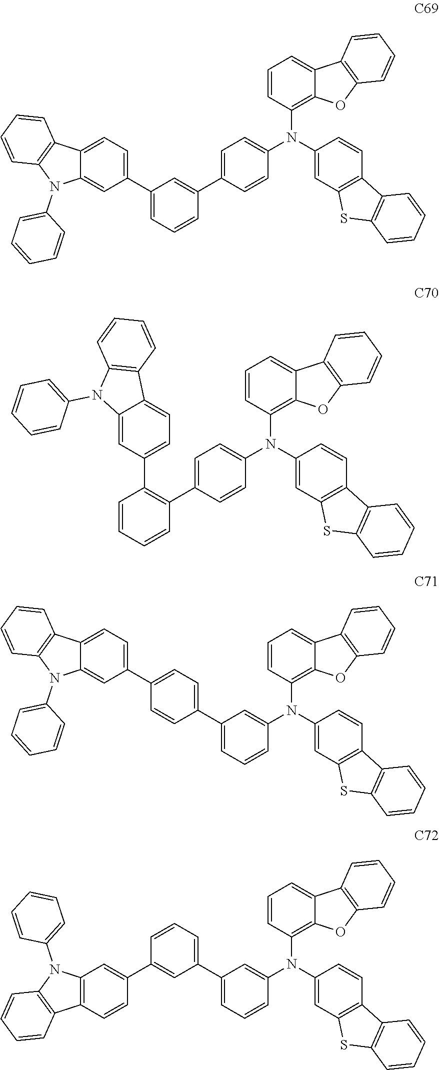 [SMILES: c1ccc(-n2c3ccccc3c3ccc(-c4cccc(-c5ccc(N(c6ccc7c(c6)sc6ccccc67)c6cccc7c6oc6ccccc67)cc5)c4)cc32)cc1 ; c1ccc(-n2c3ccccc3c3ccc(-c4ccccc4-c4ccc(N(c5ccc6c(c5)sc5ccccc56)c5cccc6c5oc5ccccc56)cc4)cc32)cc1 ; c1ccc(-n2c3ccccc3c3ccc(-c4ccc(-c5cccc(N(c6ccc7c(c6)sc6ccccc67)c6cccc7c6oc6ccccc67)c5)cc4)cc32)cc1 ; c1ccc(-n2c3ccccc3c3ccc(-c4cccc(-c5cccc(N(c6ccc7c(c6)sc6ccccc67)c6cccc7c6oc6ccccc67)c5)c4)cc32)cc1]

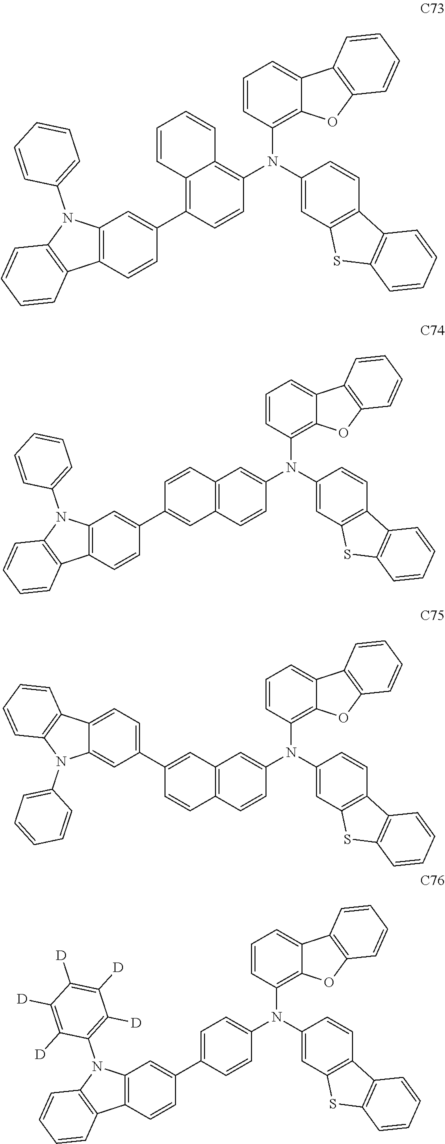 [SMILES: c1ccc(-n2c3ccccc3c3ccc(-c4ccc(N(c5ccc6c(c5)sc5ccccc56)c5cccc6c5oc5ccccc56)c5ccccc45)cc32)cc1 ; c1ccc(-n2c3ccccc3c3ccc(-c4ccc5cc(N(c6ccc7c(c6)sc6ccccc67)c6cccc7c6oc6ccccc67)ccc5c4)cc32)cc1 ; c1ccc(-n2c3ccccc3c3ccc(-c4ccc5ccc(N(c6ccc7c(c6)sc6ccccc67)c6cccc7c6oc6ccccc67)cc5c4)cc32)cc1 ; [2H]c1c([2H])c([2H])c(-n2c3ccccc3c3ccc(-c4ccc(N(c5ccc6c(c5)sc5ccccc56)c5cccc6c5oc5ccccc56)cc4)cc32)c([2H])c1[2H]]

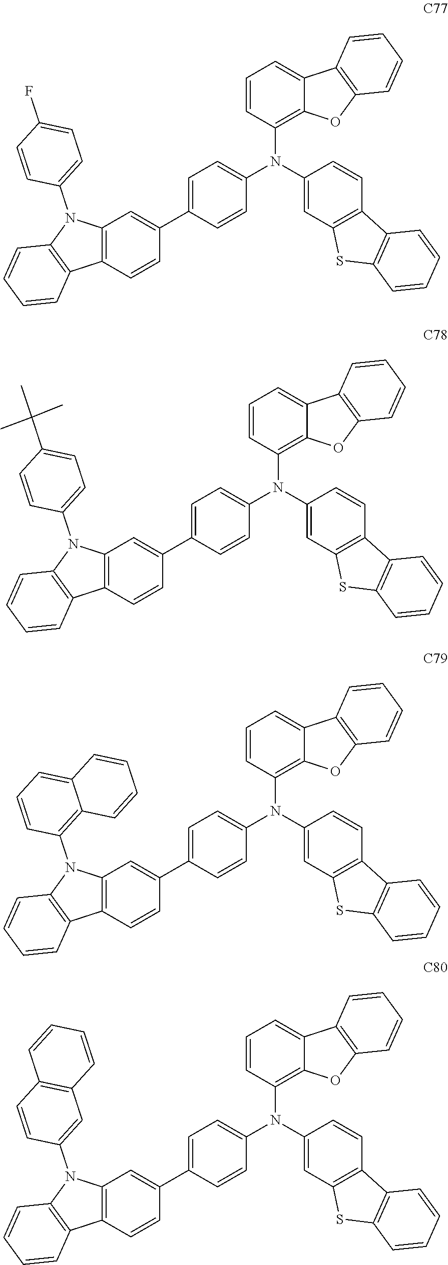 [SMILES: Fc1ccc(-n2c3ccccc3c3ccc(-c4ccc(N(c5ccc6c(c5)sc5ccccc56)c5cccc6c5oc5ccccc56)cc4)cc32)cc1 ; CC(C)(C)c1ccc(-n2c3ccccc3c3ccc(-c4ccc(N(c5ccc6c(c5)sc5ccccc56)c5cccc6c5oc5ccccc56)cc4)cc32)cc1 ; c1ccc2c(-n3c4ccccc4c4ccc(-c5ccc(N(c6ccc7c(c6)sc6ccccc67)c6cccc7c6oc6ccccc67)cc5)cc43)cccc2c1 ; c1ccc2cc(-n3c4ccccc4c4ccc(-c5ccc(N(c6ccc7c(c6)sc6ccccc67)c6cccc7c6oc6ccccc67)cc5)cc43)ccc2c1]

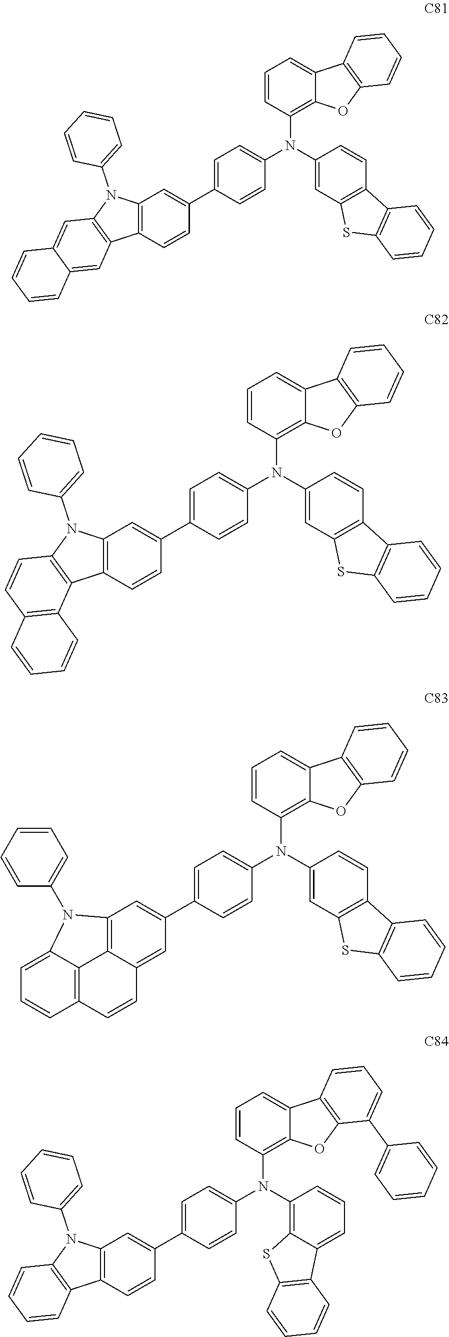 [SMILES: c1ccc(-n2c3cc(-c4ccc(N(c5ccc6c(c5)sc5ccccc56)c5cccc6c5oc5ccccc56)cc4)ccc3c3cc4ccccc4cc32)cc1 ; c1ccc(-n2c3cc(-c4ccc(N(c5ccc6c(c5)sc5ccccc56)c5cccc6c5oc5ccccc56)cc4)ccc3c3c4ccccc4ccc32)cc1 ; c1ccc(-n2c3cccc4ccc5cc(-c6ccc(N(c7ccc8c(c7)sc7ccccc78)c7cccc8c7oc7ccccc78)cc6)cc2c5c43)cc1 ; c1ccc(-c2cccc3c2oc2c(N(c4ccc(-c5ccc6c7ccccc7n(-c7ccccc7)c6c5)cc4)c4cccc5c4sc4ccccc45)cccc23)cc1]

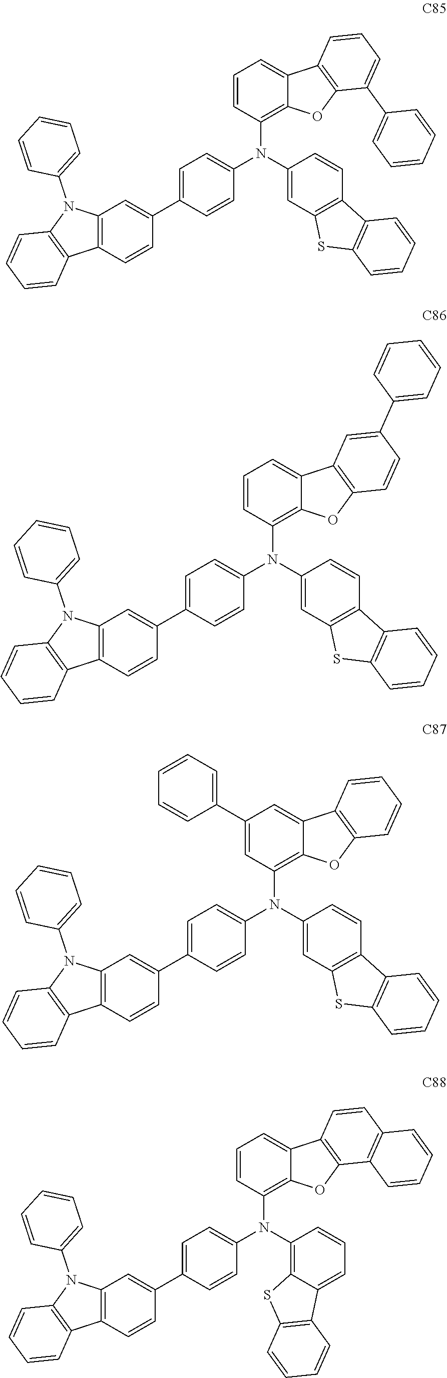 [SMILES: c1ccc(-c2cccc3c2oc2c(N(c4ccc(-c5ccc6c7ccccc7n(-c7ccccc7)c6c5)cc4)c4ccc5c(c4)sc4ccccc45)cccc23)cc1 ; c1ccc(-c2ccc3oc4c(N(c5ccc(-c6ccc7c8ccccc8n(-c8ccccc8)c7c6)cc5)c5ccc6c(c5)sc5ccccc56)cccc4c3c2)cc1 ; c1ccc(-c2cc(N(c3ccc(-c4ccc5c6ccccc6n(-c6ccccc6)c5c4)cc3)c3ccc4c(c3)sc3ccccc34)c3oc4ccccc4c3c2)cc1 ; c1ccc(-n2c3ccccc3c3ccc(-c4ccc(N(c5cccc6c5oc5c7ccccc7ccc65)c5cccc6c5sc5ccccc56)cc4)cc32)cc1]

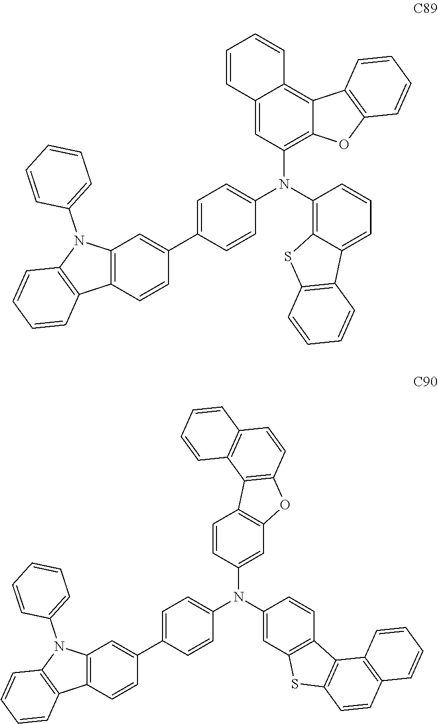 [SMILES: c1ccc(-n2c3ccccc3c3ccc(-c4ccc(N(c5cc6ccccc6c6c5oc5ccccc56)c5cccc6c5sc5ccccc56)cc4)cc32)cc1 ; c1ccc(-n2c3ccccc3c3ccc(-c4ccc(N(c5ccc6c(c5)oc5ccc7ccccc7c56)c5ccc6c(c5)sc5ccc7ccccc7c56)cc4)cc32)cc1]

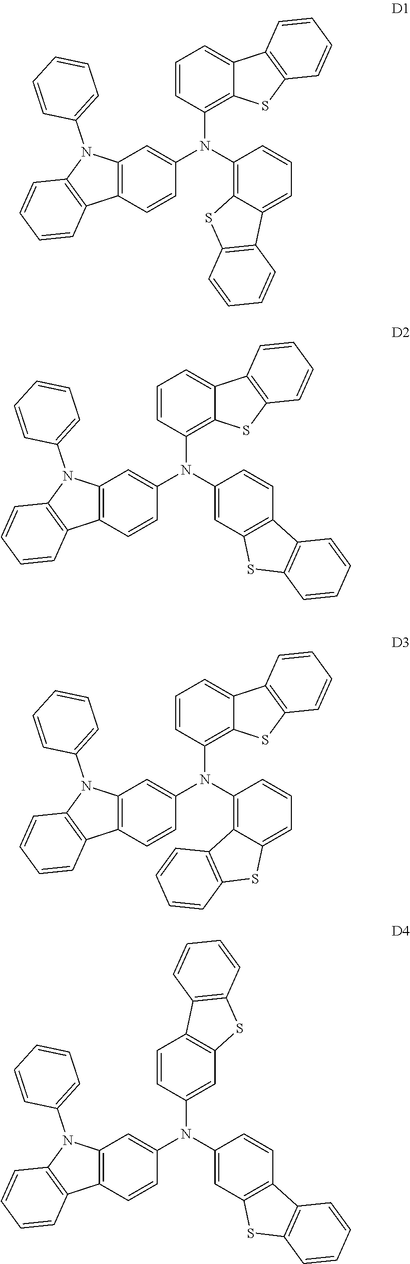

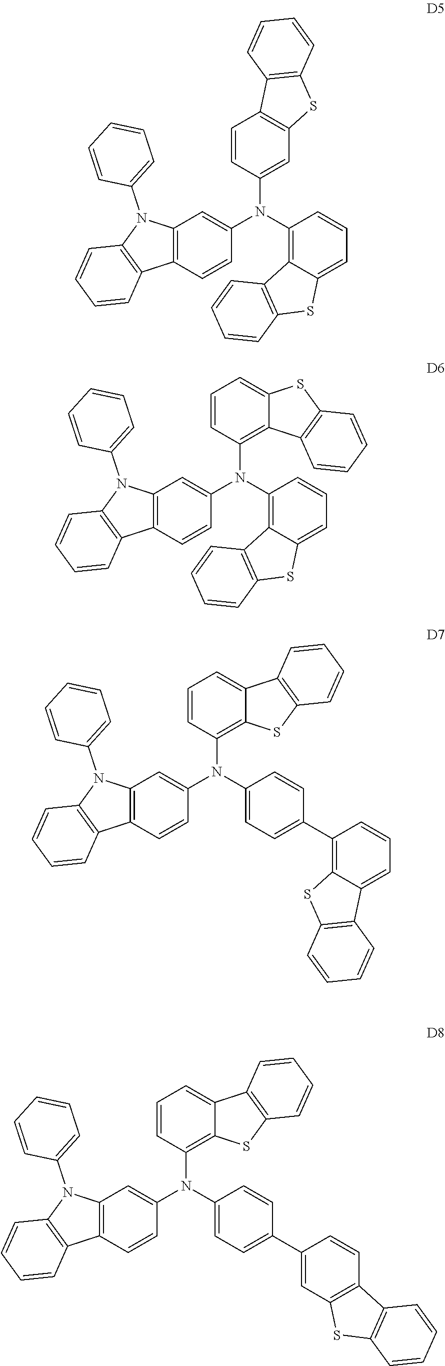

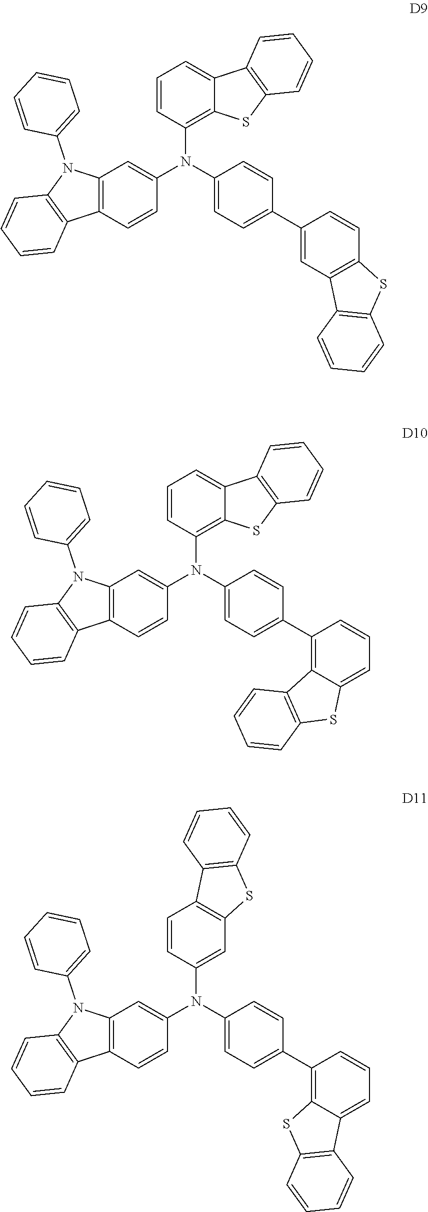 [SMILES: c1ccc(-n2c3ccccc3c3ccc(N(c4ccc(-c5ccc6sc7ccccc7c6c5)cc4)c4cccc5c4sc4ccccc45)cc32)cc1 ; c1ccc(-n2c3ccccc3c3ccc(N(c4ccc(-c5cccc6sc7ccccc7c56)cc4)c4cccc5c4sc4ccccc45)cc32)cc1 ; c1ccc(-n2c3ccccc3c3ccc(N(c4ccc(-c5cccc6c5sc5ccccc56)cc4)c4ccc5c(c4)sc4ccccc45)cc32)cc1]

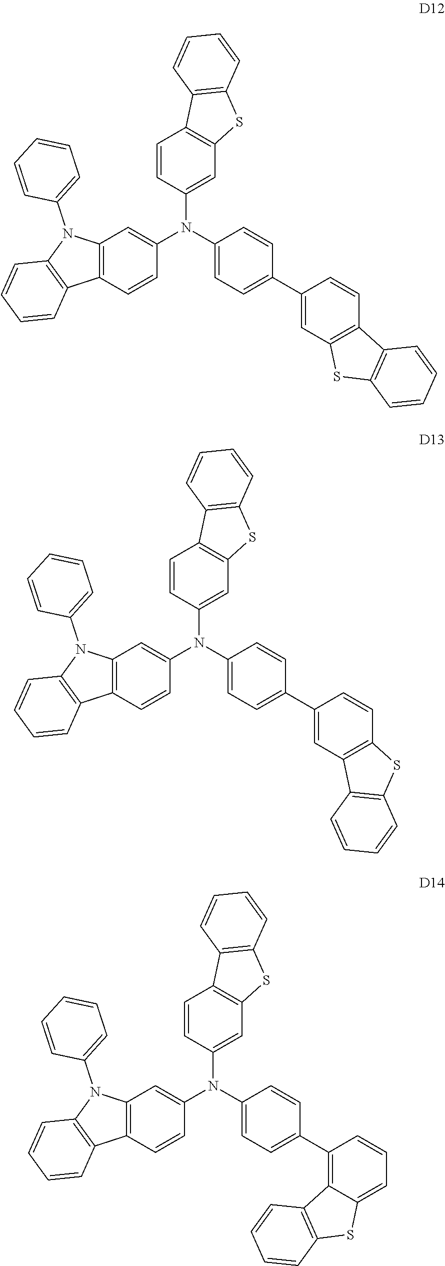 [SMILES: c1ccc(-n2c3ccccc3c3ccc(N(c4ccc(-c5ccc6c(c5)sc5ccccc56)cc4)c4ccc5c(c4)sc4ccccc45)cc32)cc1 ; c1ccc(-n2c3ccccc3c3ccc(N(c4ccc(-c5ccc6sc7ccccc7c6c5)cc4)c4ccc5c(c4)sc4ccccc45)cc32)cc1 ; c1ccc(-n2c3ccccc3c3ccc(N(c4ccc(-c5cccc6sc7ccccc7c56)cc4)c4ccc5c(c4)sc4ccccc45)cc32)cc1]

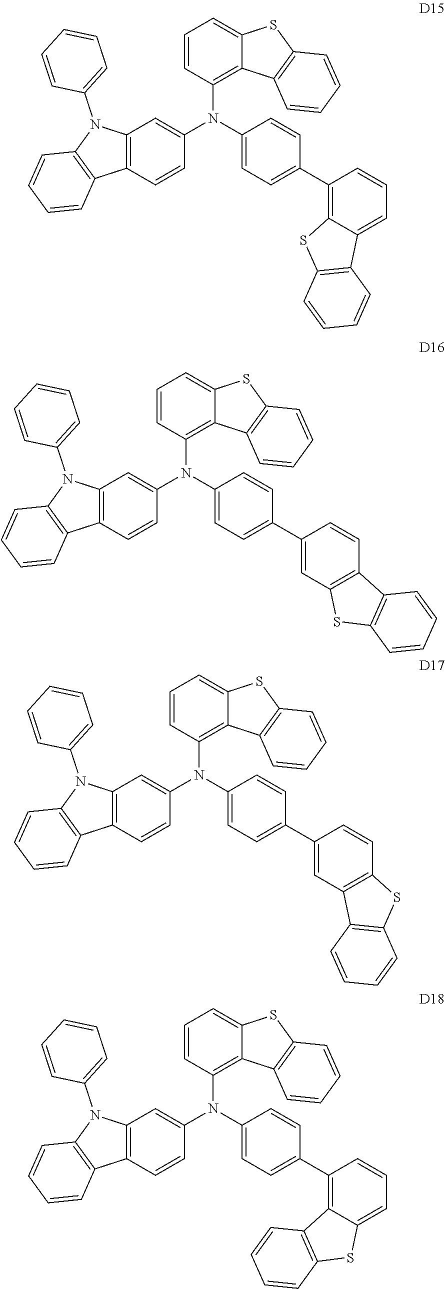 [SMILES: c1ccc(-n2c3ccccc3c3ccc(N(c4ccc(-c5cccc6c5sc5ccccc56)cc4)c4cccc5sc6ccccc6c45)cc32)cc1 ; c1ccc(-n2c3ccccc3c3ccc(N(c4ccc(-c5ccc6c(c5)sc5ccccc56)cc4)c4cccc5sc6ccccc6c45)cc32)cc1 ; c1ccc(-n2c3ccccc3c3ccc(N(c4ccc(-c5ccc6sc7ccccc7c6c5)cc4)c4cccc5sc6ccccc6c45)cc32)cc1 ; c1ccc(-n2c3ccccc3c3ccc(N(c4ccc(-c5cccc6sc7ccccc7c56)cc4)c4cccc5sc6ccccc6c45)cc32)cc1]

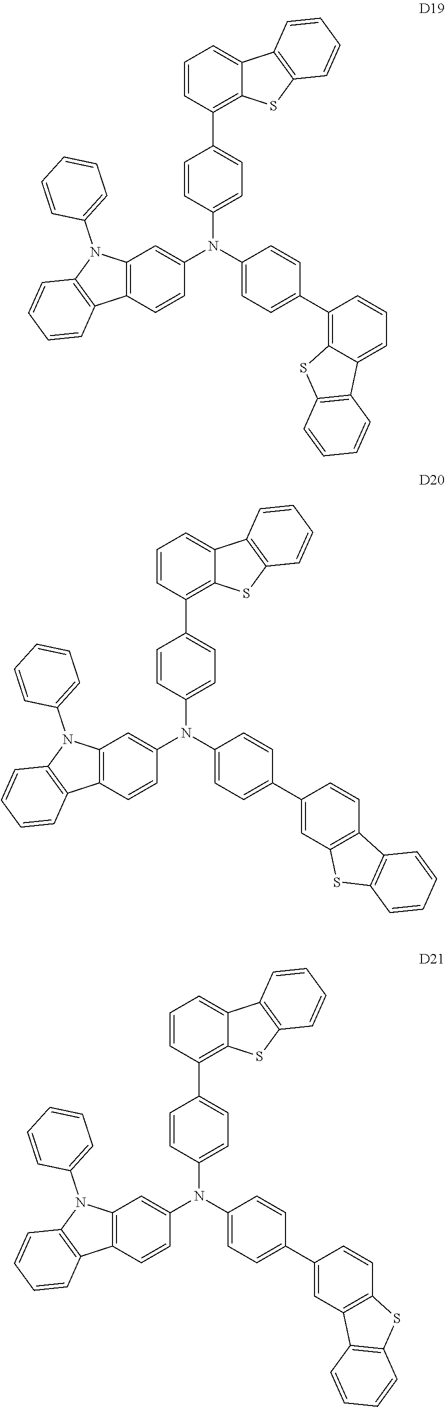 [SMILES: c1ccc(-n2c3ccccc3c3ccc(N(c4ccc(-c5cccc6c5sc5ccccc56)cc4)c4ccc(-c5cccc6c5sc5ccccc56)cc4)cc32)cc1 ; c1ccc(-n2c3ccccc3c3ccc(N(c4ccc(-c5ccc6c(c5)sc5ccccc56)cc4)c4ccc(-c5cccc6c5sc5ccccc56)cc4)cc32)cc1 ; c1ccc(-n2c3ccccc3c3ccc(N(c4ccc(-c5ccc6sc7ccccc7c6c5)cc4)c4ccc(-c5cccc6c5sc5ccccc56)cc4)cc32)cc1]

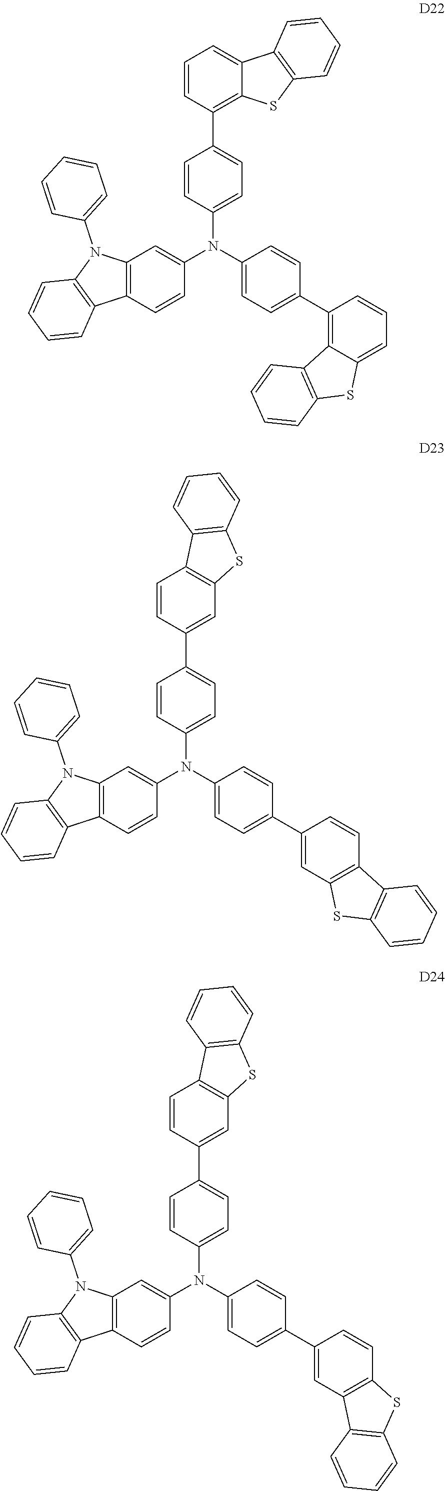 [SMILES: c1ccc(-n2c3ccccc3c3ccc(N(c4ccc(-c5cccc6c5sc5ccccc56)cc4)c4ccc(-c5cccc6sc7ccccc7c56)cc4)cc32)cc1 ; c1ccc(-n2c3ccccc3c3ccc(N(c4ccc(-c5ccc6c(c5)sc5ccccc56)cc4)c4ccc(-c5ccc6c(c5)sc5ccccc56)cc4)cc32)cc1 ; c1ccc(-n2c3ccccc3c3ccc(N(c4ccc(-c5ccc6c(c5)sc5ccccc56)cc4)c4ccc(-c5ccc6sc7ccccc7c6c5)cc4)cc32)cc1]

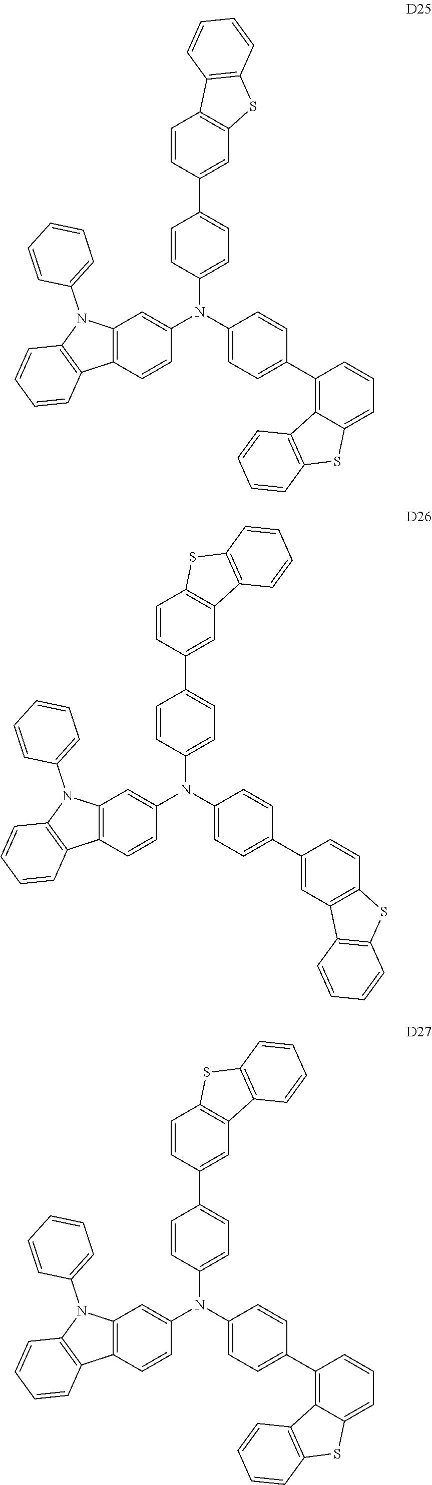

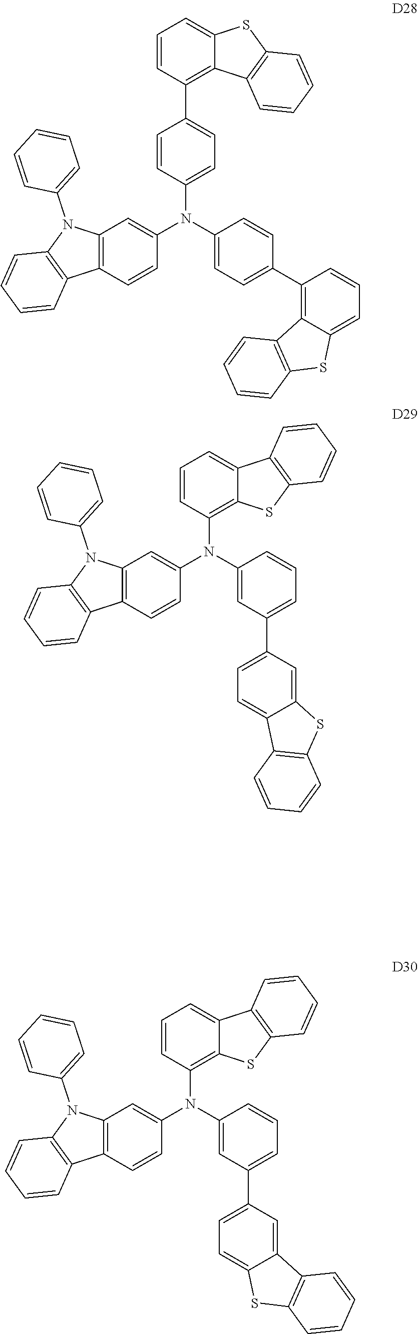

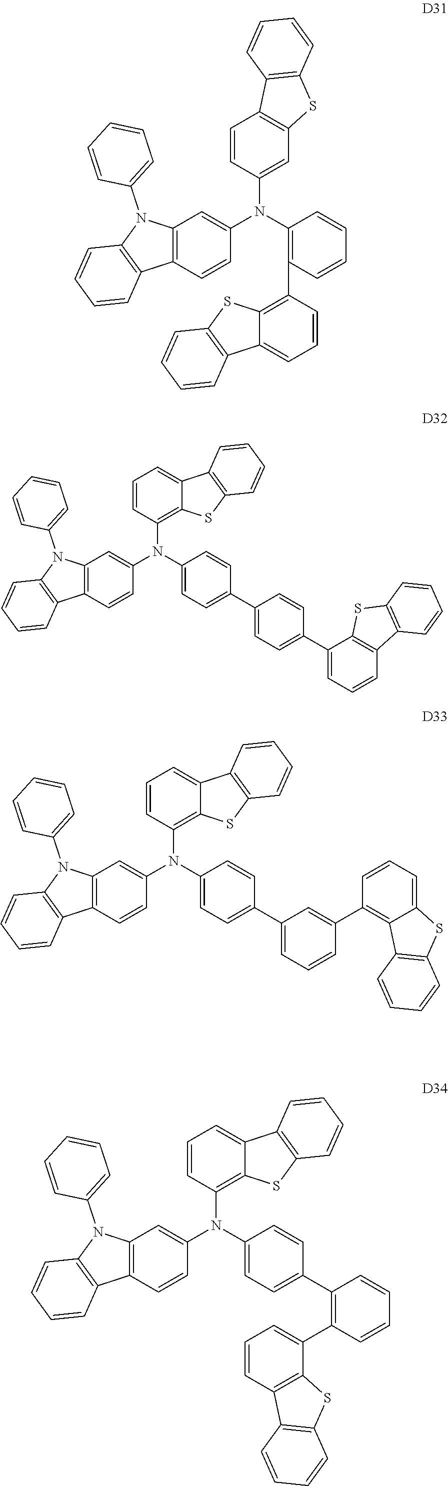 [SMILES: c1ccc(-n2c3ccccc3c3ccc(N(c4ccc5c(c4)sc4ccccc45)c4ccccc4-c4cccc5c4sc4ccccc45)cc32)cc1 ; c1ccc(-n2c3ccccc3c3ccc(N(c4ccc(-c5ccc(-c6cccc7c6sc6ccccc67)cc5)cc4)c4cccc5c4sc4ccccc45)cc32)cc1 ; c1ccc(-n2c3ccccc3c3ccc(N(c4ccc(-c5cccc(-c6cccc7sc8ccccc8c67)c5)cc4)c4cccc5c4sc4ccccc45)cc32)cc1 ; c1ccc(-n2c3ccccc3c3ccc(N(c4ccc(-c5ccccc5-c5cccc6c5sc5ccccc56)cc4)c4cccc5c4sc4ccccc45)cc32)cc1]

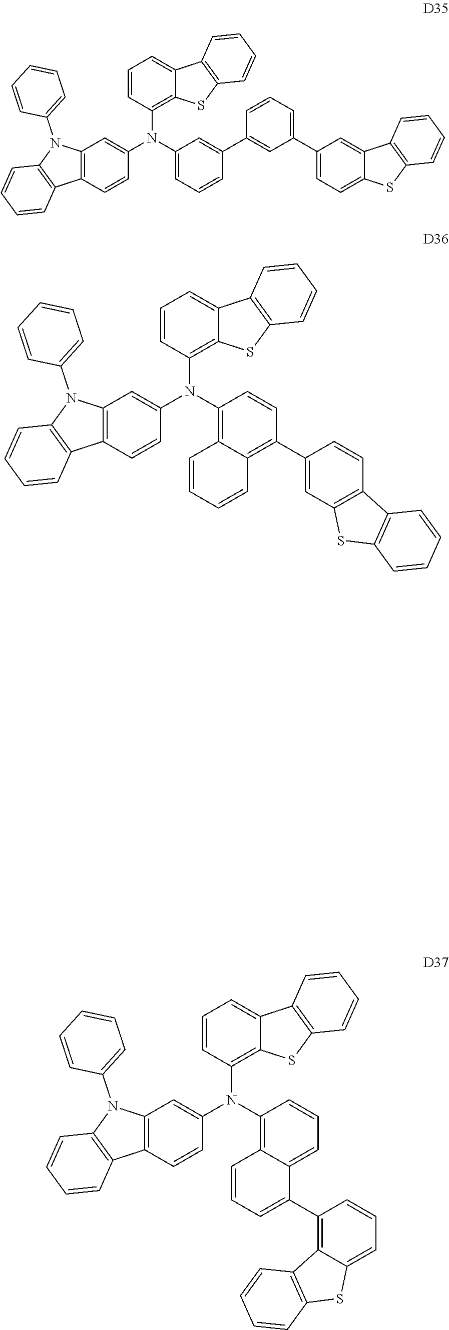 [SMILES: c1ccc(-n2c3ccccc3c3ccc(N(c4cccc(-c5cccc(-c6ccc7sc8ccccc8c7c6)c5)c4)c4cccc5c4sc4ccccc45)cc32)cc1 ; c1ccc(-n2c3ccccc3c3ccc(N(c4ccc(-c5ccc6c(c5)sc5ccccc56)c5ccccc45)c4cccc5c4sc4ccccc45)cc32)cc1 ; c1ccc(-n2c3ccccc3c3ccc(N(c4cccc5c(-c6cccc7sc8ccccc8c67)cccc45)c4cccc5c4sc4ccccc45)cc32)cc1]

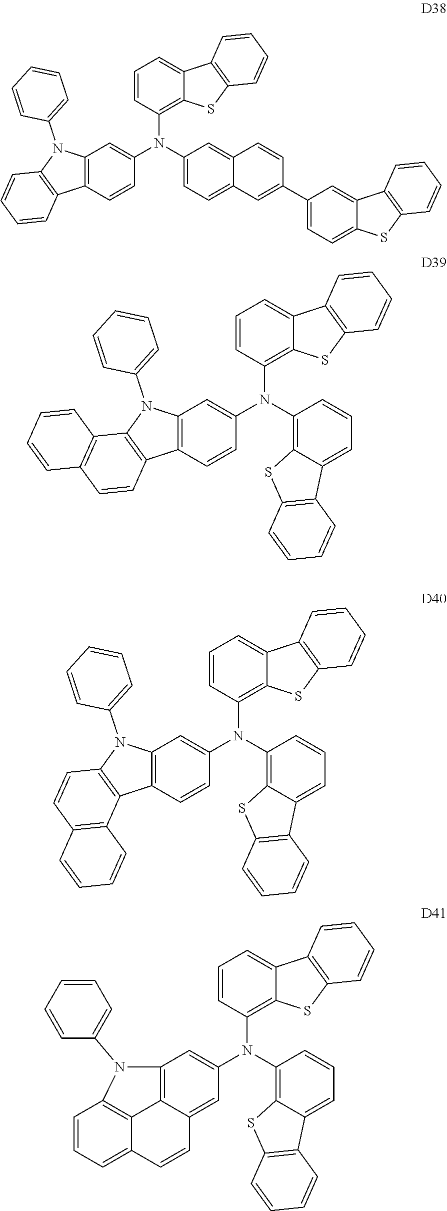

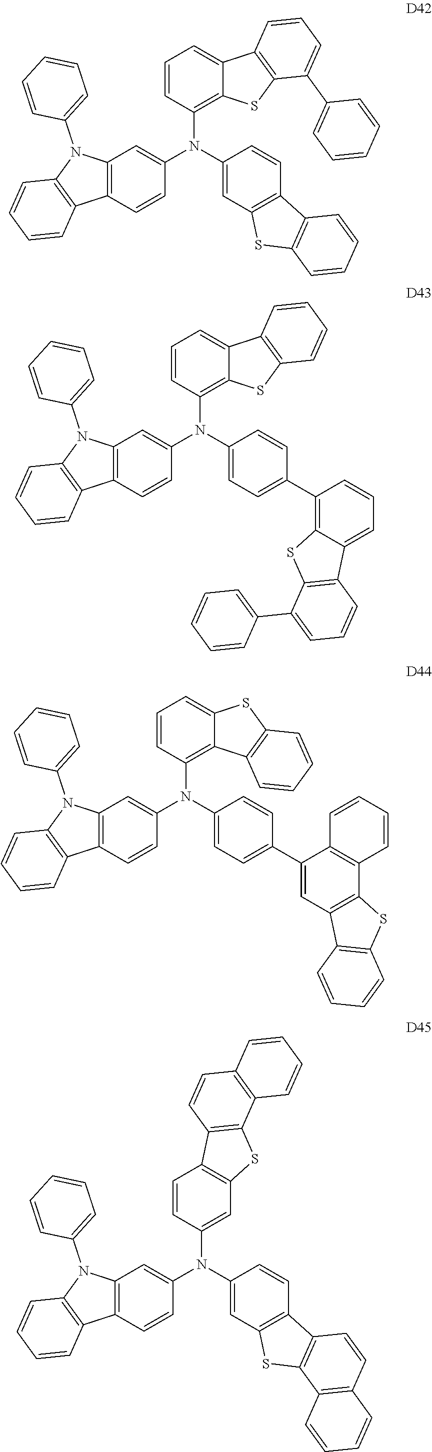

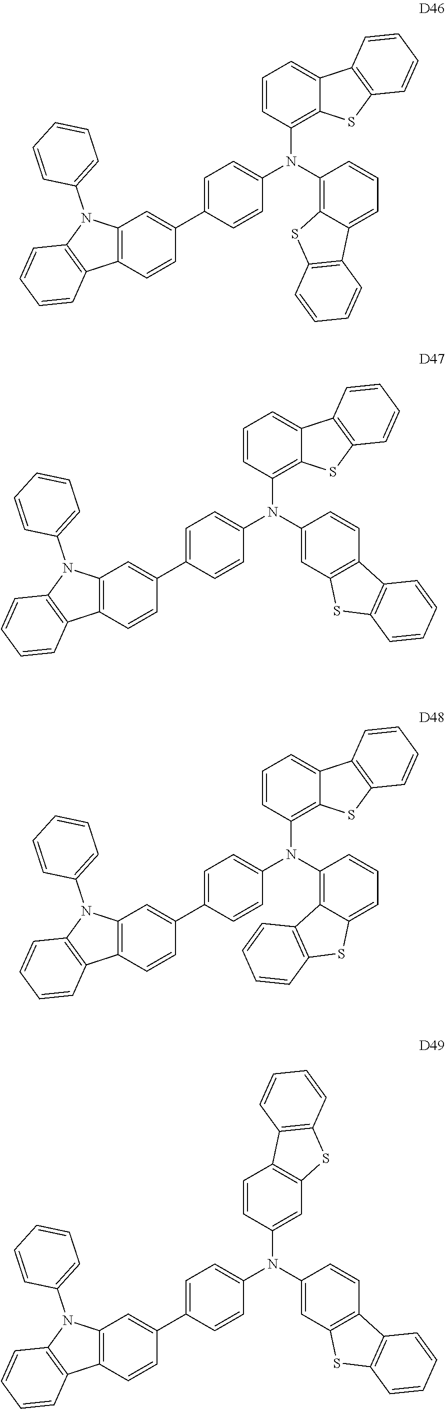 [SMILES: c1ccc(-n2c3ccccc3c3ccc(-c4ccc(N(c5cccc6c5sc5ccccc56)c5cccc6c5sc5ccccc56)cc4)cc32)cc1 ; c1ccc(-n2c3ccccc3c3ccc(-c4ccc(N(c5ccc6c(c5)sc5ccccc56)c5cccc6c5sc5ccccc56)cc4)cc32)cc1 ; c1ccc(-n2c3ccccc3c3ccc(-c4ccc(N(c5cccc6c5sc5ccccc56)c5cccc6sc7ccccc7c56)cc4)cc32)cc1 ; c1ccc(-n2c3ccccc3c3ccc(-c4ccc(N(c5ccc6c(c5)sc5ccccc56)c5ccc6c(c5)sc5ccccc56)cc4)cc32)cc1]

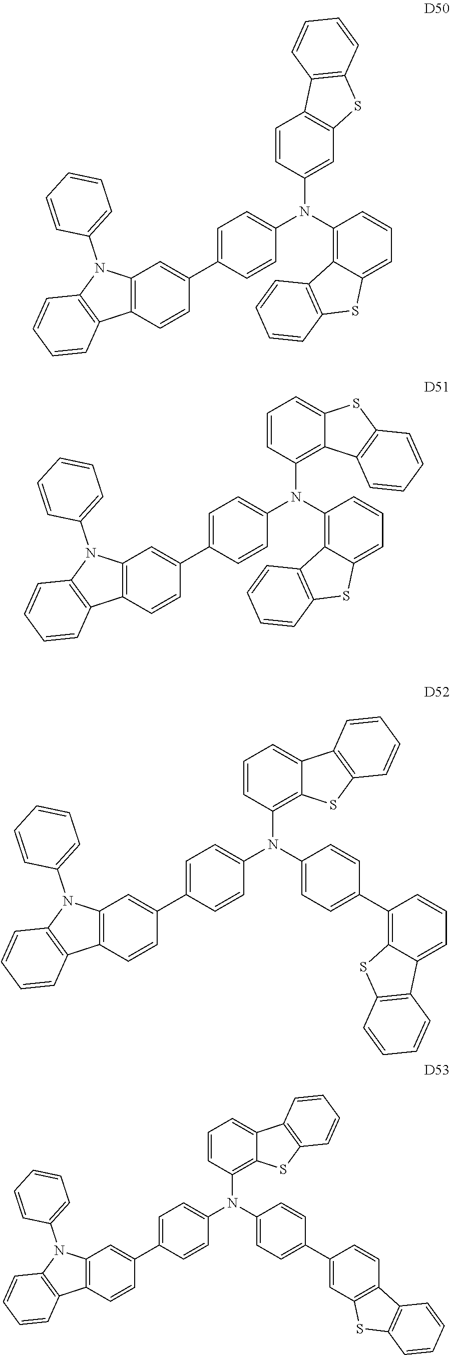 [SMILES: c1ccc(-n2c3ccccc3c3ccc(-c4ccc(N(c5ccc6c(c5)sc5ccccc56)c5cccc6sc7ccccc7c56)cc4)cc32)cc1 ; c1ccc(-n2c3ccccc3c3ccc(-c4ccc(N(c5cccc6sc7ccccc7c56)c5cccc6sc7ccccc7c56)cc4)cc32)cc1 ; c1ccc(-n2c3ccccc3c3ccc(-c4ccc(N(c5ccc(-c6cccc7c6sc6ccccc67)cc5)c5cccc6c5sc5ccccc56)cc4)cc32)cc1 ; c1ccc(-n2c3ccccc3c3ccc(-c4ccc(N(c5ccc(-c6ccc7c(c6)sc6ccccc67)cc5)c5cccc6c5sc5ccccc56)cc4)cc32)cc1]

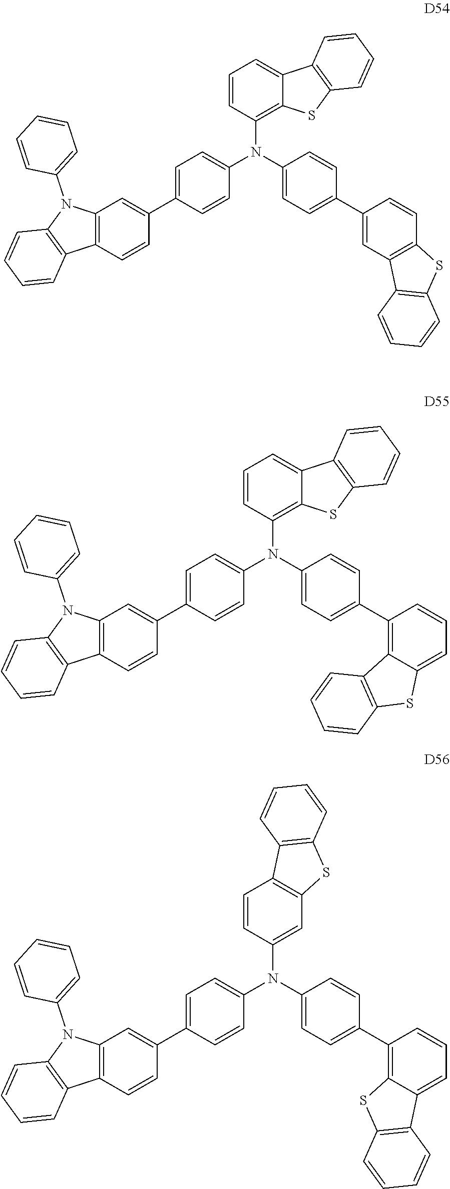 [SMILES: c1ccc(-n2c3ccccc3c3ccc(-c4ccc(N(c5ccc(-c6ccc7sc8ccccc8c7c6)cc5)c5cccc6c5sc5ccccc56)cc4)cc32)cc1 ; c1ccc(-n2c3ccccc3c3ccc(-c4ccc(N(c5ccc(-c6cccc7sc8ccccc8c67)cc5)c5cccc6c5sc5ccccc56)cc4)cc32)cc1 ; c1ccc(-n2c3ccccc3c3ccc(-c4ccc(N(c5ccc(-c6cccc7c6sc6ccccc67)cc5)c5ccc6c(c5)sc5ccccc56)cc4)cc32)cc1]

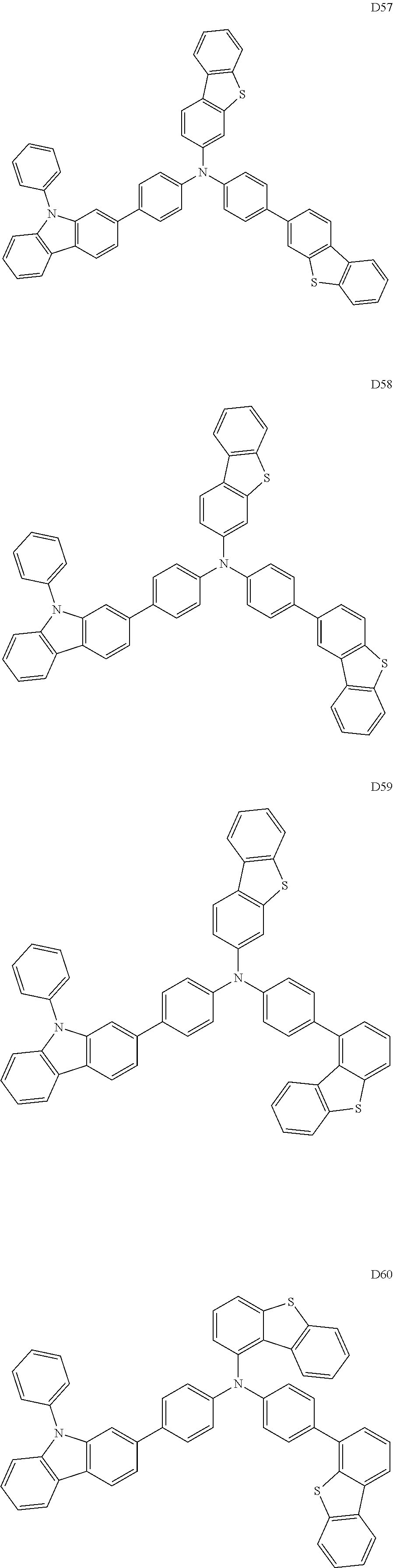 [SMILES: c1ccc(-n2c3ccccc3c3ccc(-c4ccc(N(c5ccc(-c6ccc7c(c6)sc6ccccc67)cc5)c5ccc6c(c5)sc5ccccc56)cc4)cc32)cc1 ; c1ccc(-n2c3ccccc3c3ccc(-c4ccc(N(c5ccc(-c6ccc7sc8ccccc8c7c6)cc5)c5ccc6c(c5)sc5ccccc56)cc4)cc32)cc1 ; c1ccc(-n2c3ccccc3c3ccc(-c4ccc(N(c5ccc(-c6cccc7sc8ccccc8c67)cc5)c5ccc6c(c5)sc5ccccc56)cc4)cc32)cc1 ; c1ccc(-n2c3ccccc3c3ccc(-c4ccc(N(c5ccc(-c6cccc7c6sc6ccccc67)cc5)c5cccc6sc7ccccc7c56)cc4)cc32)cc1]

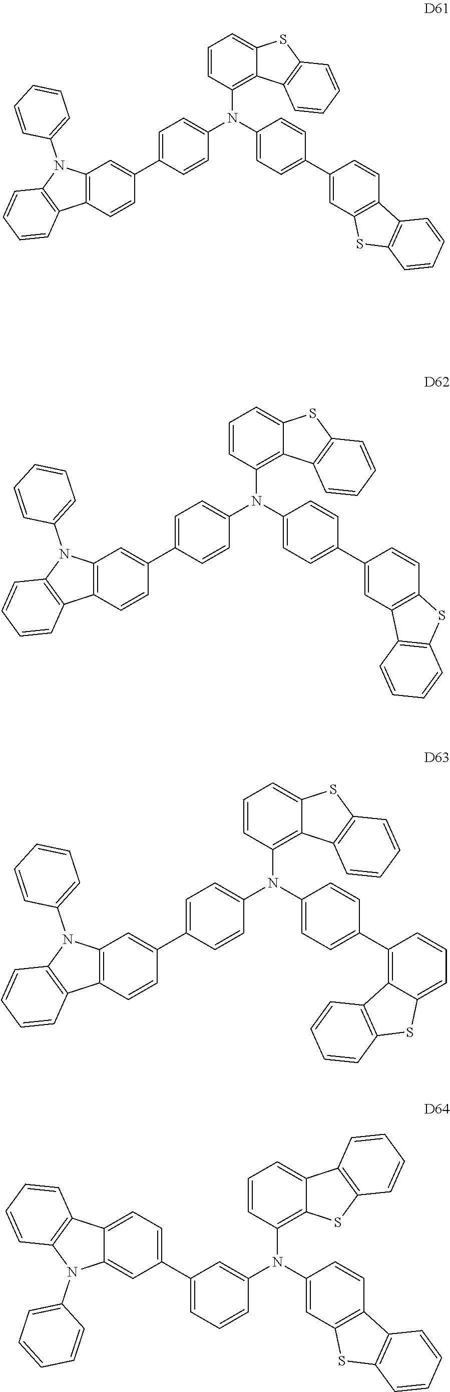 [SMILES: c1ccc(-n2c3ccccc3c3ccc(-c4ccc(N(c5ccc(-c6ccc7c(c6)sc6ccccc67)cc5)c5cccc6sc7ccccc7c56)cc4)cc32)cc1 ; c1ccc(-n2c3ccccc3c3ccc(-c4ccc(N(c5ccc(-c6ccc7sc8ccccc8c7c6)cc5)c5cccc6sc7ccccc7c56)cc4)cc32)cc1 ; c1ccc(-n2c3ccccc3c3ccc(-c4ccc(N(c5ccc(-c6cccc7sc8ccccc8c67)cc5)c5cccc6sc7ccccc7c56)cc4)cc32)cc1 ; c1ccc(-n2c3ccccc3c3ccc(-c4cccc(N(c5ccc6c(c5)sc5ccccc56)c5cccc6c5sc5ccccc56)c4)cc32)cc1]

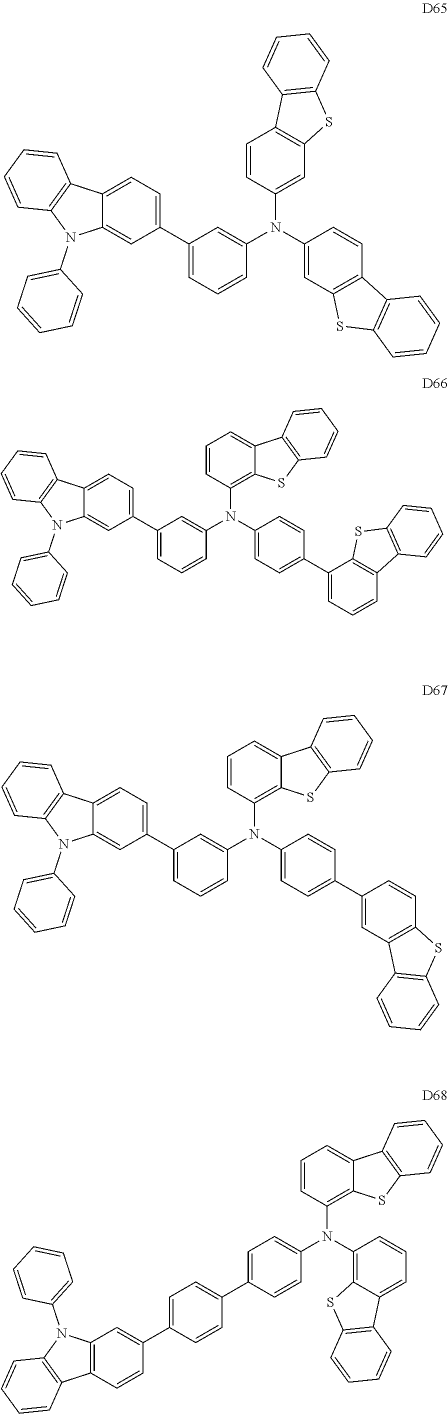 [SMILES: c1ccc(-n2c3ccccc3c3ccc(-c4cccc(N(c5ccc6c(c5)sc5ccccc56)c5ccc6c(c5)sc5ccccc56)c4)cc32)cc1 ; c1ccc(-n2c3ccccc3c3ccc(-c4cccc(N(c5ccc(-c6cccc7c6sc6ccccc67)cc5)c5cccc6c5sc5ccccc56)c4)cc32)cc1 ; c1ccc(-n2c3ccccc3c3ccc(-c4cccc(N(c5ccc(-c6ccc7sc8ccccc8c7c6)cc5)c5cccc6c5sc5ccccc56)c4)cc32)cc1 ; c1ccc(-n2c3ccccc3c3ccc(-c4ccc(-c5ccc(N(c6cccc7c6sc6ccccc67)c6cccc7c6sc6ccccc67)cc5)cc4)cc32)cc1]

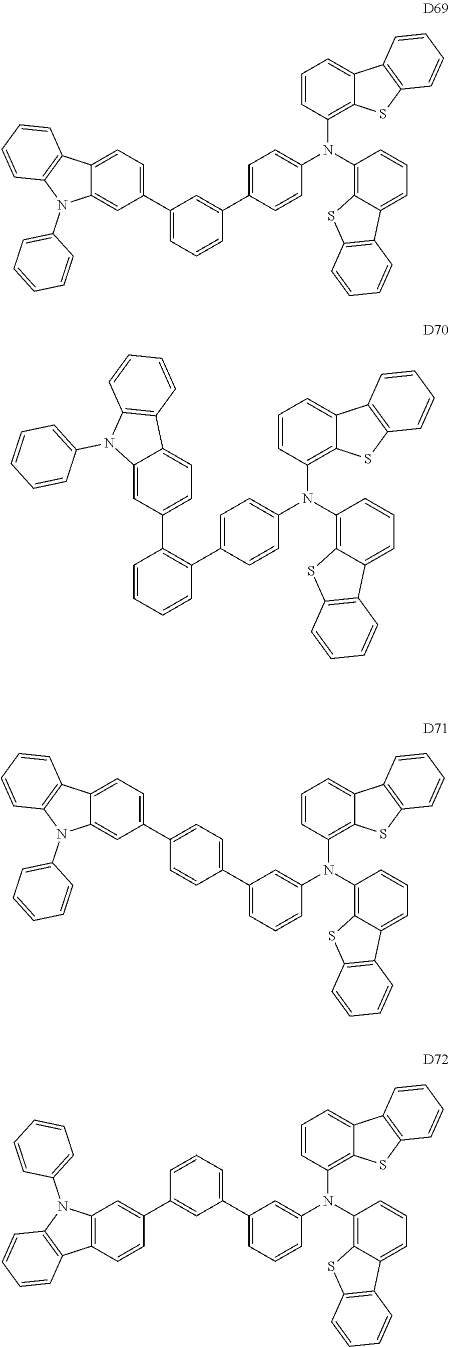 [SMILES: c1ccc(-n2c3ccccc3c3ccc(-c4cccc(-c5ccc(N(c6cccc7c6sc6ccccc67)c6cccc7c6sc6ccccc67)cc5)c4)cc32)cc1 ; c1ccc(-n2c3ccccc3c3ccc(-c4ccccc4-c4ccc(N(c5cccc6c5sc5ccccc56)c5cccc6c5sc5ccccc56)cc4)cc32)cc1 ; c1ccc(-n2c3ccccc3c3ccc(-c4ccc(-c5cccc(N(c6cccc7c6sc6ccccc67)c6cccc7c6sc6ccccc67)c5)cc4)cc32)cc1 ; c1ccc(-n2c3ccccc3c3ccc(-c4cccc(-c5cccc(N(c6cccc7c6sc6ccccc67)c6cccc7c6sc6ccccc67)c5)c4)cc32)cc1]

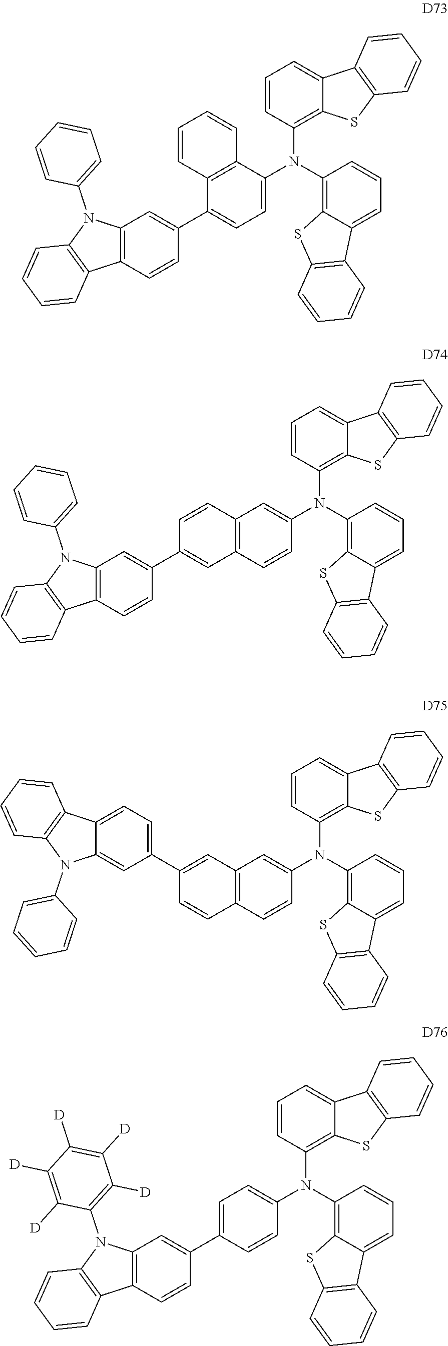 [SMILES: c1ccc(-n2c3ccccc3c3ccc(-c4ccc(N(c5cccc6c5sc5ccccc56)c5cccc6c5sc5ccccc56)c5ccccc45)cc32)cc1 ; c1ccc(-n2c3ccccc3c3ccc(-c4ccc5cc(N(c6cccc7c6sc6ccccc67)c6cccc7c6sc6ccccc67)ccc5c4)cc32)cc1 ; c1ccc(-n2c3ccccc3c3ccc(-c4ccc5ccc(N(c6cccc7c6sc6ccccc67)c6cccc7c6sc6ccccc67)cc5c4)cc32)cc1 ; [2H]c1c([2H])c([2H])c(-n2c3ccccc3c3ccc(-c4ccc(N(c5cccc6c5sc5ccccc56)c5cccc6c5sc5ccccc56)cc4)cc32)c([2H])c1[2H]]

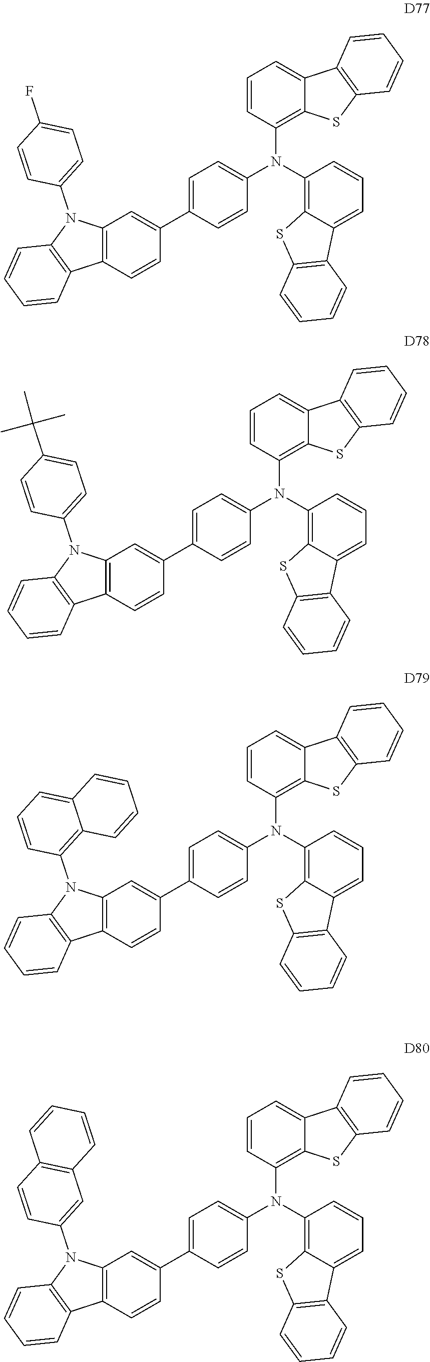 [SMILES: Fc1ccc(-n2c3ccccc3c3ccc(-c4ccc(N(c5cccc6c5sc5ccccc56)c5cccc6c5sc5ccccc56)cc4)cc32)cc1 ; CC(C)(C)c1ccc(-n2c3ccccc3c3ccc(-c4ccc(N(c5cccc6c5sc5ccccc56)c5cccc6c5sc5ccccc56)cc4)cc32)cc1 ; c1ccc2c(-n3c4ccccc4c4ccc(-c5ccc(N(c6cccc7c6sc6ccccc67)c6cccc7c6sc6ccccc67)cc5)cc43)cccc2c1 ; c1ccc2cc(-n3c4ccccc4c4ccc(-c5ccc(N(c6cccc7c6sc6ccccc67)c6cccc7c6sc6ccccc67)cc5)cc43)ccc2c1]

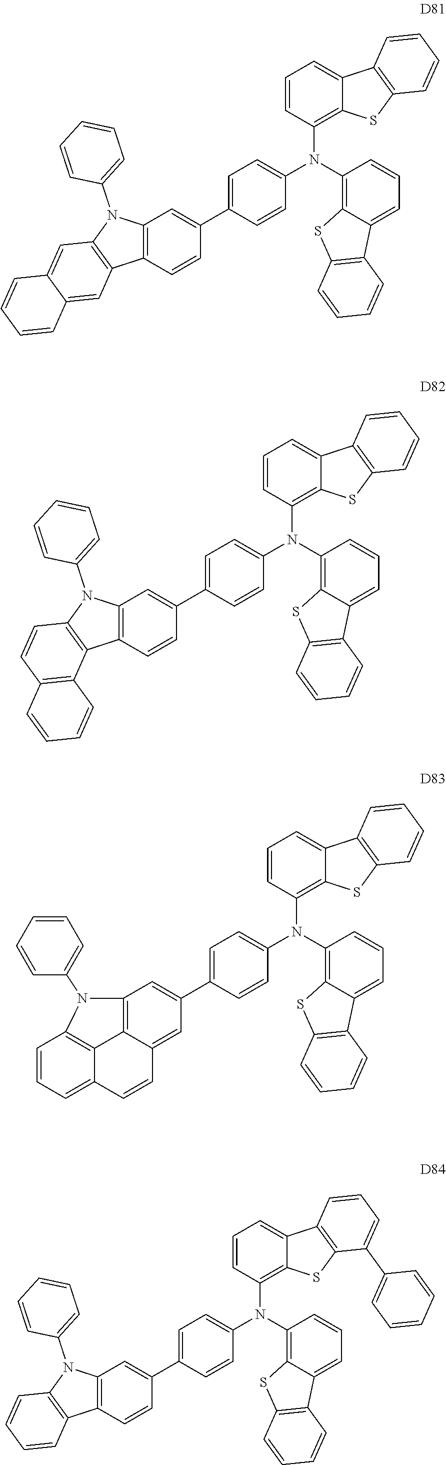 [SMILES: c1ccc(-n2c3cc(-c4ccc(N(c5cccc6c5sc5ccccc56)c5cccc6c5sc5ccccc56)cc4)ccc3c3cc4ccccc4cc32)cc1 ; c1ccc(-n2c3cc(-c4ccc(N(c5cccc6c5sc5ccccc56)c5cccc6c5sc5ccccc56)cc4)ccc3c3c4ccccc4ccc32)cc1 ; c1ccc(-n2c3cccc4ccc5cc(-c6ccc(N(c7cccc8c7sc7ccccc78)c7cccc8c7sc7ccccc78)cc6)cc2c5c43)cc1 ; c1ccc(-c2cccc3c2sc2c(N(c4ccc(-c5ccc6c7ccccc7n(-c7ccccc7)c6c5)cc4)c4cccc5c4sc4ccccc45)cccc23)cc1]

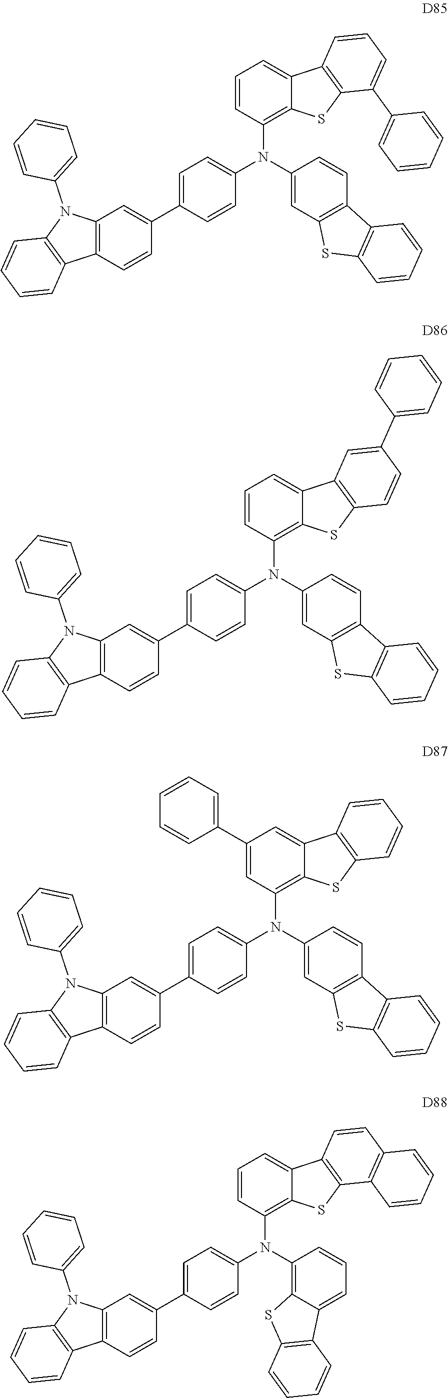 [SMILES: c1ccc(-c2cccc3c2sc2c(N(c4ccc(-c5ccc6c7ccccc7n(-c7ccccc7)c6c5)cc4)c4ccc5c(c4)sc4ccccc45)cccc23)cc1 ; c1ccc(-c2ccc3sc4c(N(c5ccc(-c6ccc7c8ccccc8n(-c8ccccc8)c7c6)cc5)c5ccc6c(c5)sc5ccccc56)cccc4c3c2)cc1 ; c1ccc(-c2cc(N(c3ccc(-c4ccc5c6ccccc6n(-c6ccccc6)c5c4)cc3)c3ccc4c(c3)sc3ccccc34)c3sc4ccccc4c3c2)cc1 ; c1ccc(-n2c3ccccc3c3ccc(-c4ccc(N(c5cccc6c5sc5ccccc56)c5cccc6c5sc5c7ccccc7ccc65)cc4)cc32)cc1]

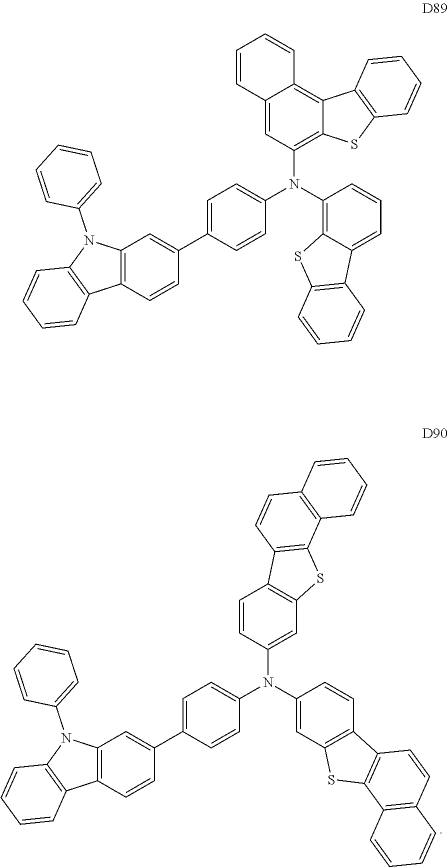 [SMILES: c1ccc(-n2c3ccccc3c3ccc(-c4ccc(N(c5cccc6c5sc5ccccc56)c5cc6ccccc6c6c5sc5ccccc56)cc4)cc32)cc1 ; c1ccc(-n2c3ccccc3c3ccc(-c4ccc(N(c5ccc6c(c5)sc5c7ccccc7ccc65)c5ccc6c(c5)sc5c7ccccc7ccc65)cc4)cc32)cc1]

13. An amine compound represented by Formula 1: 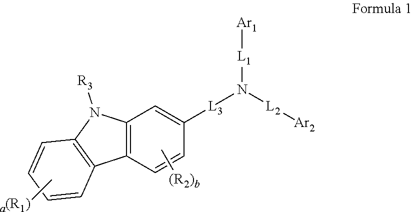
wherein, in Formula 1,
R₁ and R₂ are each independently a hydrogen atom, a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, and/or are bonded to an adjacent group to form a ring,
when R₁ and R₂ are each a substituted silyl group, a substituted alkyl group, a substituted alkenyl group, or a substituted aryl group, the substituent is not an amine group,
R₃ is an unsubstituted aryl group having 6 to 10 ring-forming carbon atoms, or an aryl group having 6 to 10 ring-forming carbon atoms substituted with a deuterium atom, a halogen atom, or a substituted or unsubstituted alkyl group having 1 to 20 carbon atoms,
a is an integer from 0 to 4, and b is an integer from 0 to 3,
at least one selected from among L₁ to L₃ is a direct linkage, and the L₁ to L₃ is that are not a direct linkage are represented by any one selected from among Formula 2-1 to Formula 2-3, and
Ar₁ and Ar₂ are each independently represented by Formula 3: 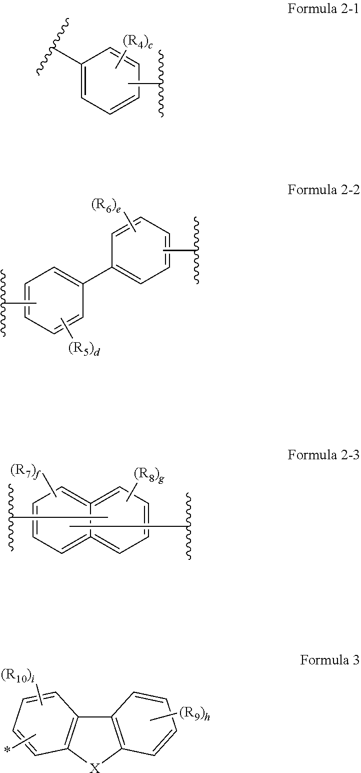
wherein, in Formula 2-1 to Formula 2-3,
R₄ to R₈ are each independently a hydrogen atom, a deuterium atom, a halogen atom, or a substituted or unsubstituted alkyl group having 1 to 20 carbon atoms, and
c, d, e, f, and g are each independently an integer from 0 to 4, and
in Formula 3,
X is O or S,
R₉ and R₁₀ are each independently a hydrogen atom, a deuterium atom, a halogen atom, a substituted or unsubstituted silyl group, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, and/or adjacent R₉s or adjacent R₁₀s are bonded to each other to form an aromatic hydrocarbon ring,
when adjacent R₉s or adjacent R₁₀s are bonded to each other to form an aromatic hydrocarbon ring, only one pair of R₉s or R₁₀s selected from among a plurality of R₉s and R₁₀s are bonded to each other to form an aromatic hydrocarbon ring,
h is an integer from 0 to 4, and i is an integer from 0 to 3,
“-*” is a position linked to L₁ and L₂, and
when L₁ and L₂ are each a direct linkage, “-*” is not linked to a nitrogen atom in the para relation to X.

14. The amine compound of claim 13, wherein R₁ and R₂ are each independently a hydrogen atom or a deuterium atom, and/or are bonded to an adjacent group to form an aromatic hydrocarbon ring.

15. The amine compound of claim 13, wherein R₃ is represented by any one selected from among Substituent Group S1: 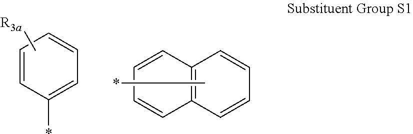
wherein, in Substituent Group S1,
R₃ₐ is a hydrogen atom, a deuterium atom, an alkyl group having 1 to 10 carbon atoms, or a halogen atom.

16. The amine compound of claim 13, wherein Formula 3 is represented by Formula 3-1 or Formula 3-2: 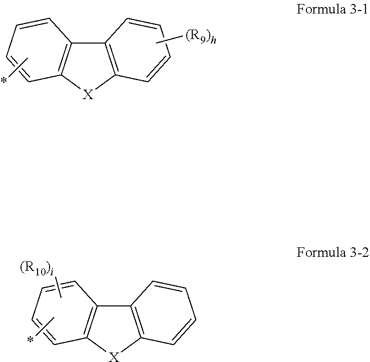
wherein, in Formula 3-1 and Formula 3-2,
R₉, R₁₀, X, h, and i are the same as defined in Formula 3.

17. The amine compound of claim 13, wherein Formula 3 is represented by any one selected from among 3-a to 3-k:

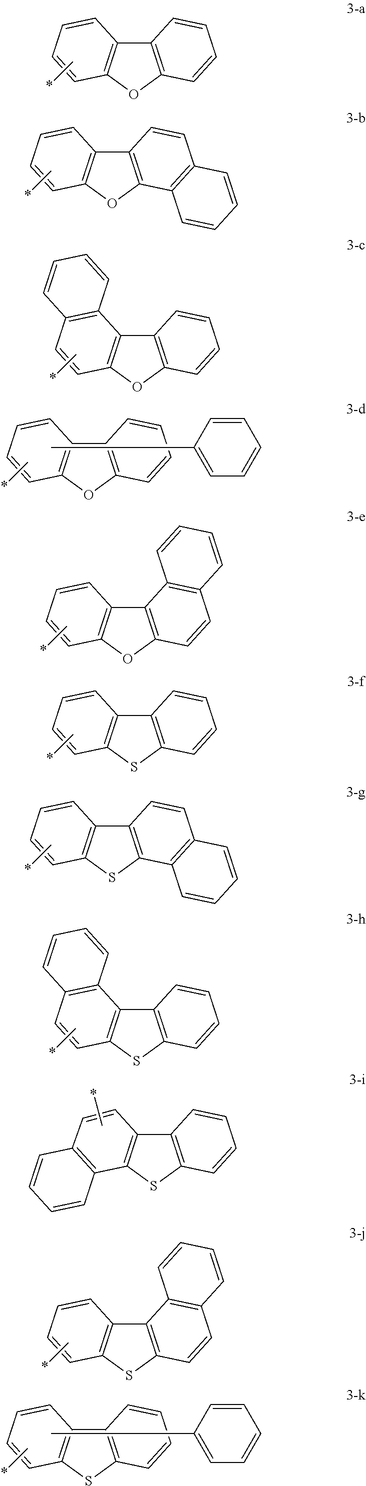

18. The amine compound of claim 13, wherein Formula 1 is represented by Formula 4: 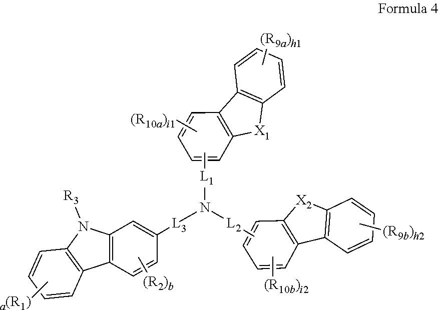
wherein, in Formula 4,
X₁ and X₂ are each independently O or S,
R₉ₐ and R₁₀ₐ are each independently a hydrogen atom, a deuterium atom, or a substituted or unsubstituted aryl group having 6 to 30 ring-forming carbon atoms, or adjacent R₉ₐs or adjacent R₁₀ₐs are bonded to each other to form an aromatic ring,
R_{9b} and R_{10b} are each independently a hydrogen atom, a deuterium atom, or a substituted or unsubstituted aryl group having 6 to 30 ring-forming carbon atoms, or adjacent R_{9b}s or adjacent R_{10b}s are bonded to each other to form an aromatic ring,
h1 and h2 are each independently an integer from 0 to 4,
i1 and i2 are each independently an integer from 0 to 3, and
R₁ to R₃, a, b, and L₁ to L₃ are the same as defined in Formula 1.

19. The amine compound of claim 18, wherein Formula 4 is represented by any one selected from among Formula 4-1 to Formula 4-5: 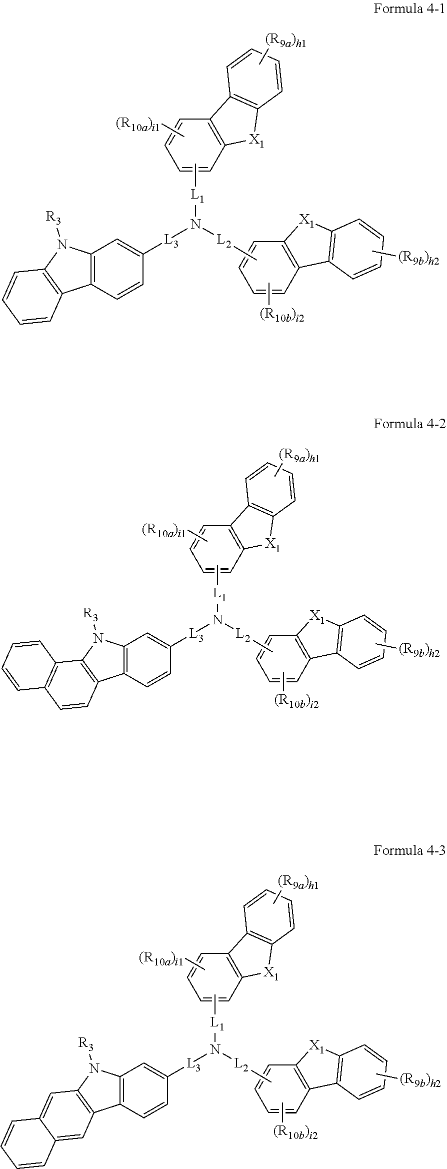 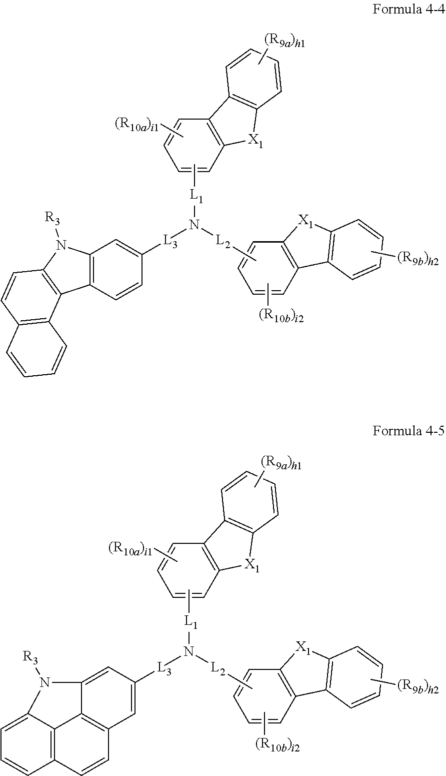
wherein, in Formula 4-1 to Formula 4-5,
R₃, and L₁ to L₃ are the same as defined in Formula 1, and
X₁, X₂, R₉ₐ, R_{9b}, R₁₀ₐ, R_{10b}, h1, h2, i1, and i2 are the same as defined in Formula 4.

20. The amine compound of claim 13, wherein the amine compound represented by Formula 1 is any one selected from among compounds of Compound Group 1: 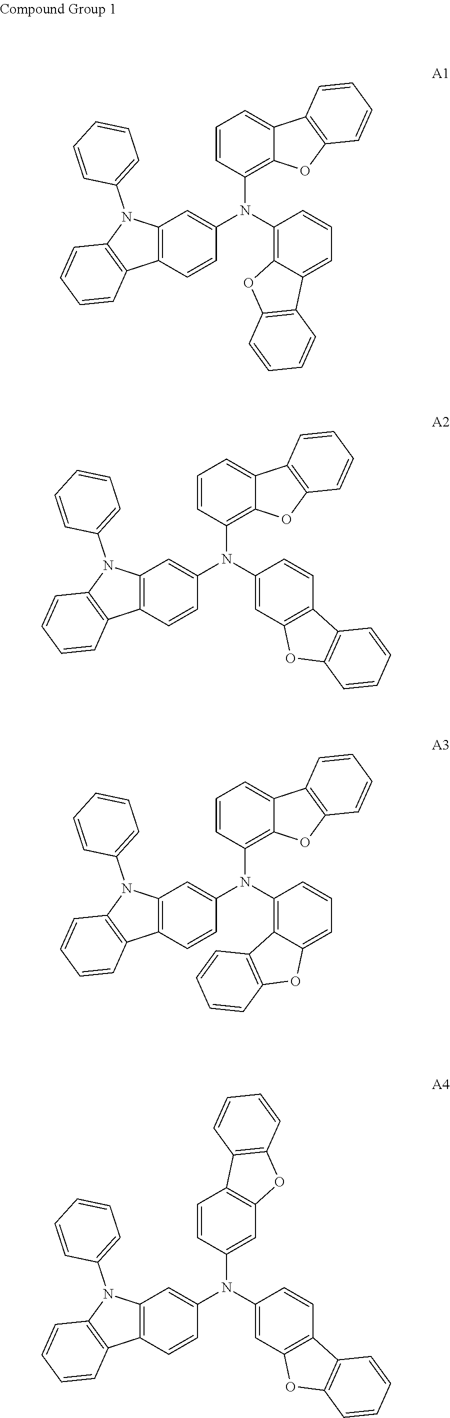 [SMILES: c1ccc(-n2c3ccccc3c3ccc(N(c4cccc5c4oc4ccccc45)c4cccc5c4oc4ccccc45)cc32)cc1 ; c1ccc(-n2c3ccccc3c3ccc(N(c4ccc5c(c4)oc4ccccc45)c4cccc5c4oc4ccccc45)cc32)cc1 ; c1ccc(-n2c3ccccc3c3ccc(N(c4cccc5c4oc4ccccc45)c4cccc5oc6ccccc6c45)cc32)cc1 ; c1ccc(-n2c3ccccc3c3ccc(N(c4ccc5c(c4)oc4ccccc45)c4ccc5c(c4)oc4ccccc45)cc32)cc1] 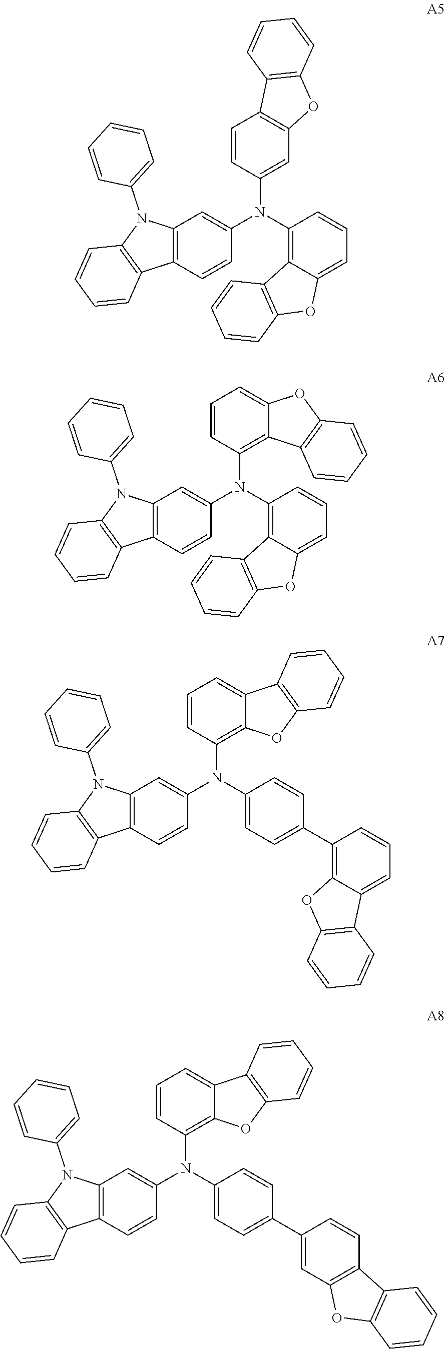 [SMILES: c1ccc(-n2c3ccccc3c3ccc(N(c4ccc5c(c4)oc4ccccc45)c4cccc5oc6ccccc6c45)cc32)cc1 ; c1ccc(-n2c3ccccc3c3ccc(N(c4cccc5oc6ccccc6c45)c4cccc5oc6ccccc6c45)cc32)cc1 ; c1ccc(-n2c3ccccc3c3ccc(N(c4ccc(-c5cccc6c5oc5ccccc56)cc4)c4cccc5c4oc4ccccc45)cc32)cc1 ; c1ccc(-n2c3ccccc3c3ccc(N(c4ccc(-c5ccc6c(c5)oc5ccccc56)cc4)c4cccc5c4oc4ccccc45)cc32)cc1] 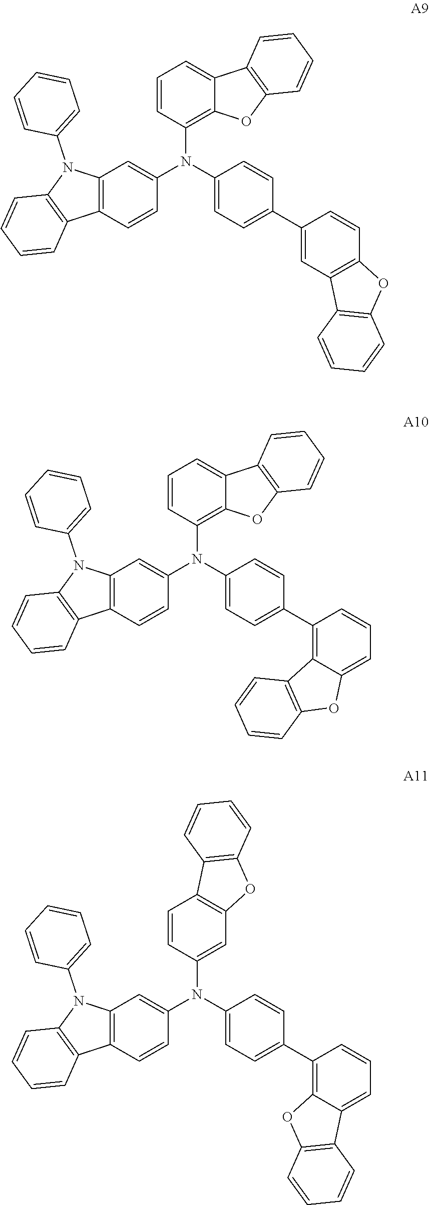 [SMILES: c1ccc(-n2c3ccccc3c3ccc(N(c4ccc(-c5ccc6oc7ccccc7c6c5)cc4)c4cccc5c4oc4ccccc45)cc32)cc1 ; c1ccc(-n2c3ccccc3c3ccc(N(c4ccc(-c5cccc6oc7ccccc7c56)cc4)c4cccc5c4oc4ccccc45)cc32)cc1 ; c1ccc(-n2c3ccccc3c3ccc(N(c4ccc(-c5cccc6c5oc5ccccc56)cc4)c4ccc5c(c4)oc4ccccc45)cc32)cc1] 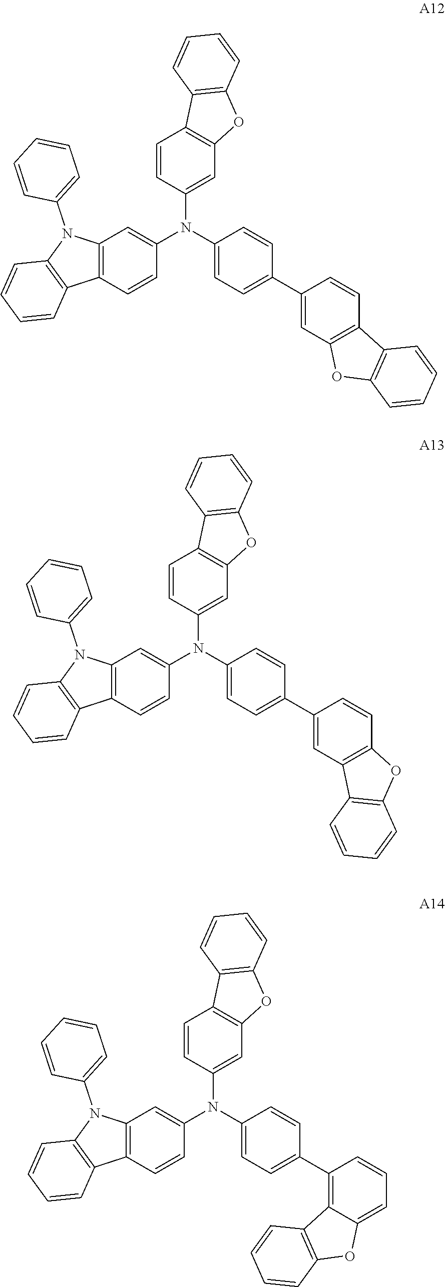 [SMILES: c1ccc(-n2c3ccccc3c3ccc(N(c4ccc(-c5ccc6c(c5)oc5ccccc56)cc4)c4ccc5c(c4)oc4ccccc45)cc32)cc1 ; c1ccc(-n2c3ccccc3c3ccc(N(c4ccc(-c5ccc6oc7ccccc7c6c5)cc4)c4ccc5c(c4)oc4ccccc45)cc32)cc1 ; c1ccc(-n2c3ccccc3c3ccc(N(c4ccc(-c5cccc6oc7ccccc7c56)cc4)c4ccc5c(c4)oc4ccccc45)cc32)cc1] 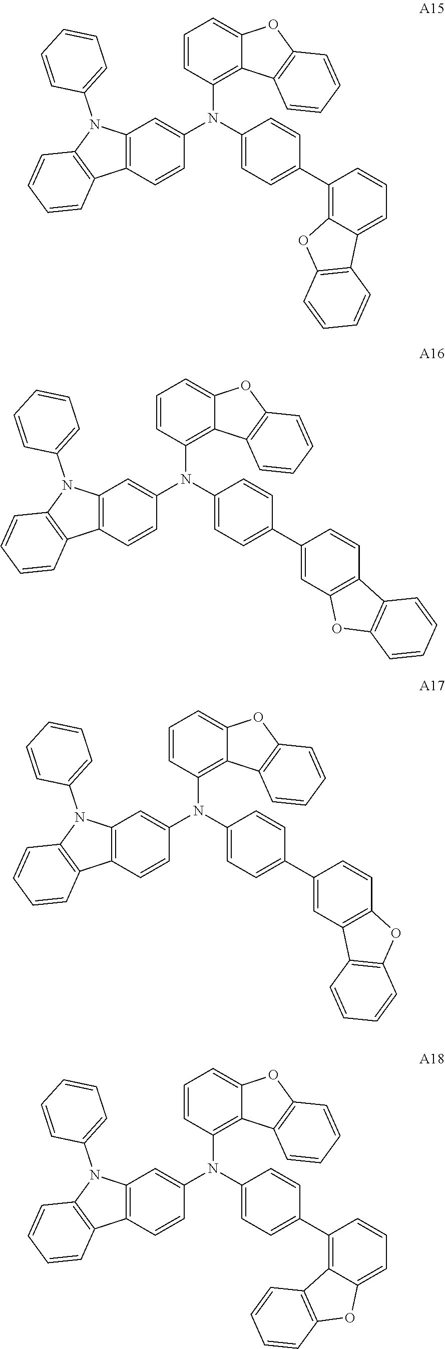 [SMILES: c1ccc(-n2c3ccccc3c3ccc(N(c4ccc(-c5cccc6c5oc5ccccc56)cc4)c4cccc5oc6ccccc6c45)cc32)cc1 ; c1ccc(-n2c3ccccc3c3ccc(N(c4ccc(-c5ccc6c(c5)oc5ccccc56)cc4)c4cccc5oc6ccccc6c45)cc32)cc1 ; c1ccc(-n2c3ccccc3c3ccc(N(c4ccc(-c5ccc6oc7ccccc7c6c5)cc4)c4cccc5oc6ccccc6c45)cc32)cc1 ; c1ccc(-n2c3ccccc3c3ccc(N(c4ccc(-c5cccc6oc7ccccc7c56)cc4)c4cccc5oc6ccccc6c45)cc32)cc1] 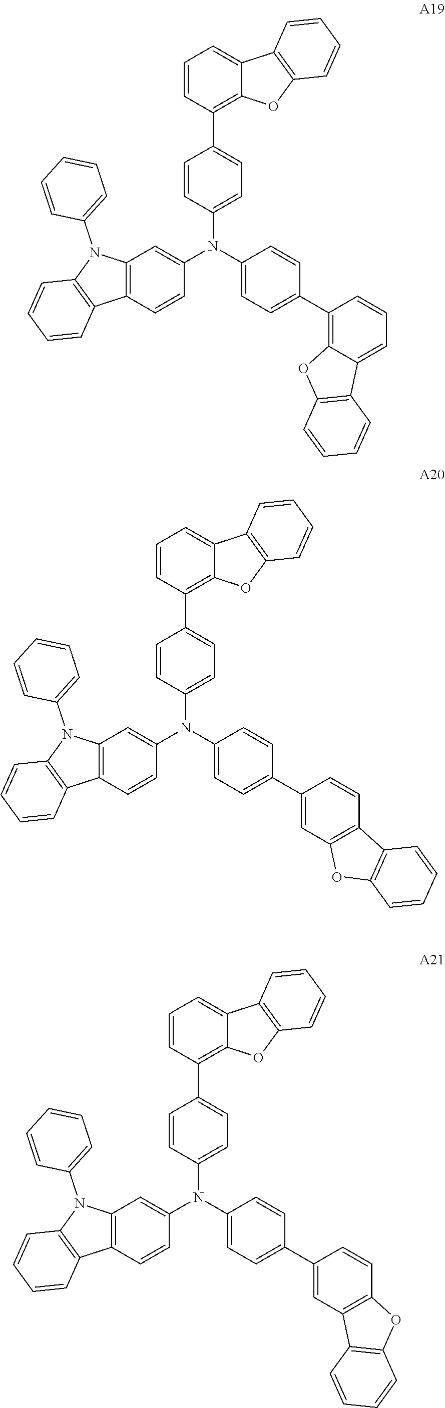 [SMILES: c1ccc(-n2c3ccccc3c3ccc(N(c4ccc(-c5cccc6c5oc5ccccc56)cc4)c4ccc(-c5cccc6c5oc5ccccc56)cc4)cc32)cc1 ; c1ccc(-n2c3ccccc3c3ccc(N(c4ccc(-c5ccc6c(c5)oc5ccccc56)cc4)c4ccc(-c5cccc6c5oc5ccccc56)cc4)cc32)cc1 ; c1ccc(-n2c3ccccc3c3ccc(N(c4ccc(-c5ccc6oc7ccccc7c6c5)cc4)c4ccc(-c5cccc6c5oc5ccccc56)cc4)cc32)cc1] 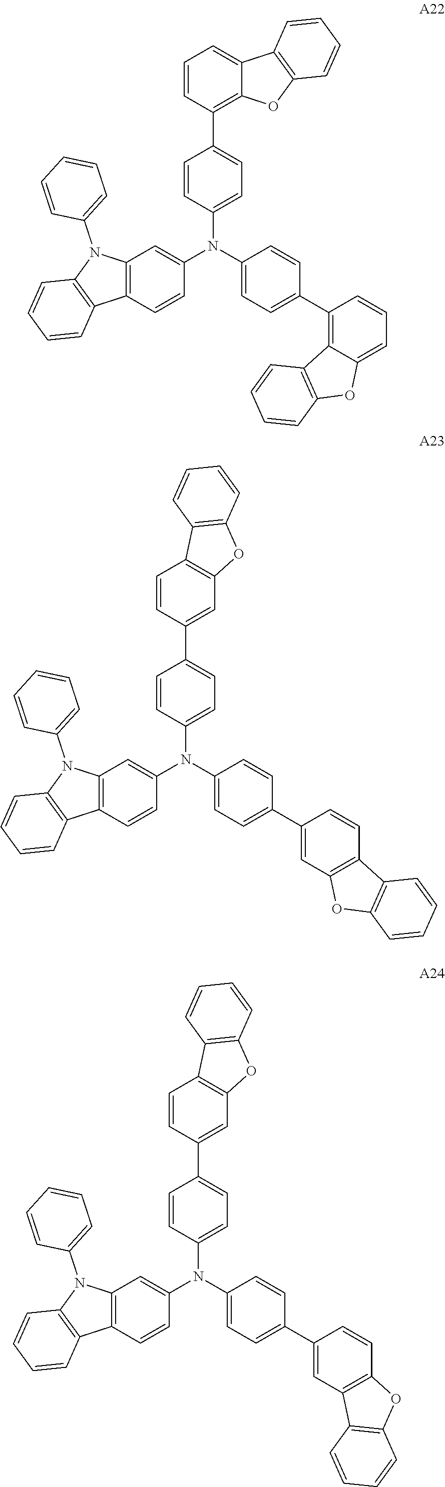 [SMILES: c1ccc(-n2c3ccccc3c3ccc(N(c4ccc(-c5cccc6c5oc5ccccc56)cc4)c4ccc(-c5cccc6oc7ccccc7c56)cc4)cc32)cc1 ; c1ccc(-n2c3ccccc3c3ccc(N(c4ccc(-c5ccc6c(c5)oc5ccccc56)cc4)c4ccc(-c5ccc6c(c5)oc5ccccc56)cc4)cc32)cc1 ; c1ccc(-n2c3ccccc3c3ccc(N(c4ccc(-c5ccc6c(c5)oc5ccccc56)cc4)c4ccc(-c5ccc6oc7ccccc7c6c5)cc4)cc32)cc1] 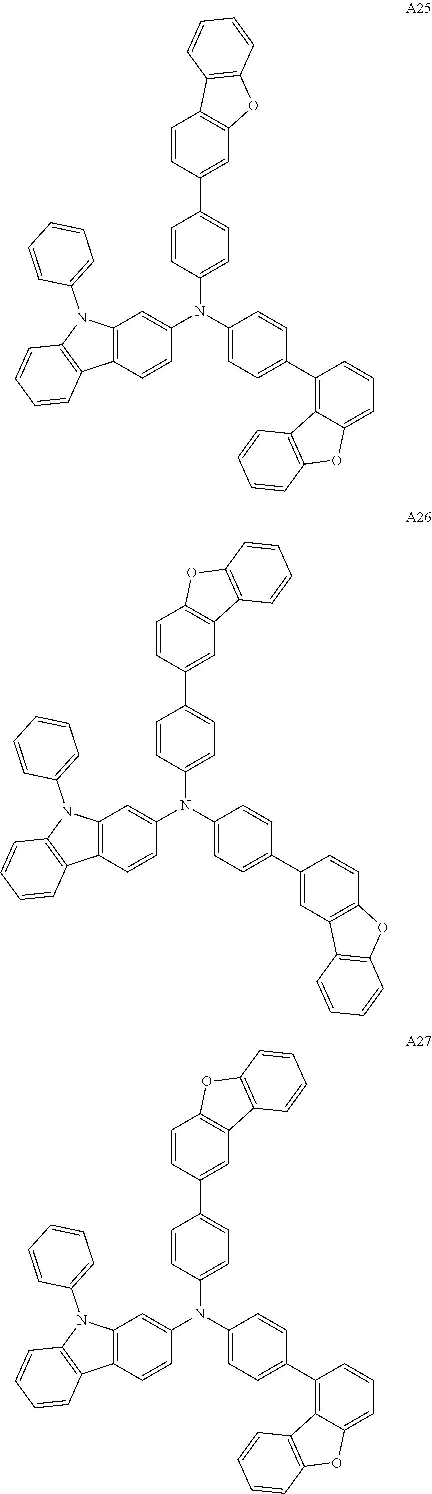 [SMILES: c1ccc(-n2c3ccccc3c3ccc(N(c4ccc(-c5ccc6c(c5)oc5ccccc56)cc4)c4ccc(-c5cccc6oc7ccccc7c56)cc4)cc32)cc1 ; c1ccc(-n2c3ccccc3c3ccc(N(c4ccc(-c5ccc6oc7ccccc7c6c5)cc4)c4ccc(-c5ccc6oc7ccccc7c6c5)cc4)cc32)cc1 ; c1ccc(-n2c3ccccc3c3ccc(N(c4ccc(-c5ccc6oc7ccccc7c6c5)cc4)c4ccc(-c5cccc6oc7ccccc7c56)cc4)cc32)cc1] 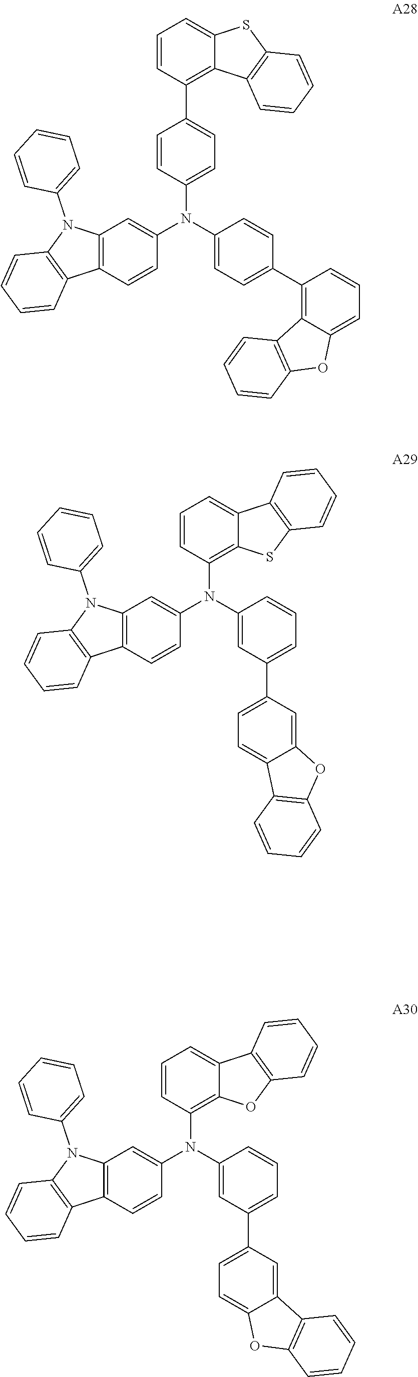 [SMILES: c1ccc(-n2c3ccccc3c3ccc(N(c4ccc(-c5cccc6oc7ccccc7c56)cc4)c4ccc(-c5cccc6sc7ccccc7c56)cc4)cc32)cc1 ; c1ccc(-n2c3ccccc3c3ccc(N(c4cccc(-c5ccc6c(c5)oc5ccccc56)c4)c4cccc5c4sc4ccccc45)cc32)cc1 ; c1ccc(-n2c3ccccc3c3ccc(N(c4cccc(-c5ccc6oc7ccccc7c6c5)c4)c4cccc5c4oc4ccccc45)cc32)cc1] 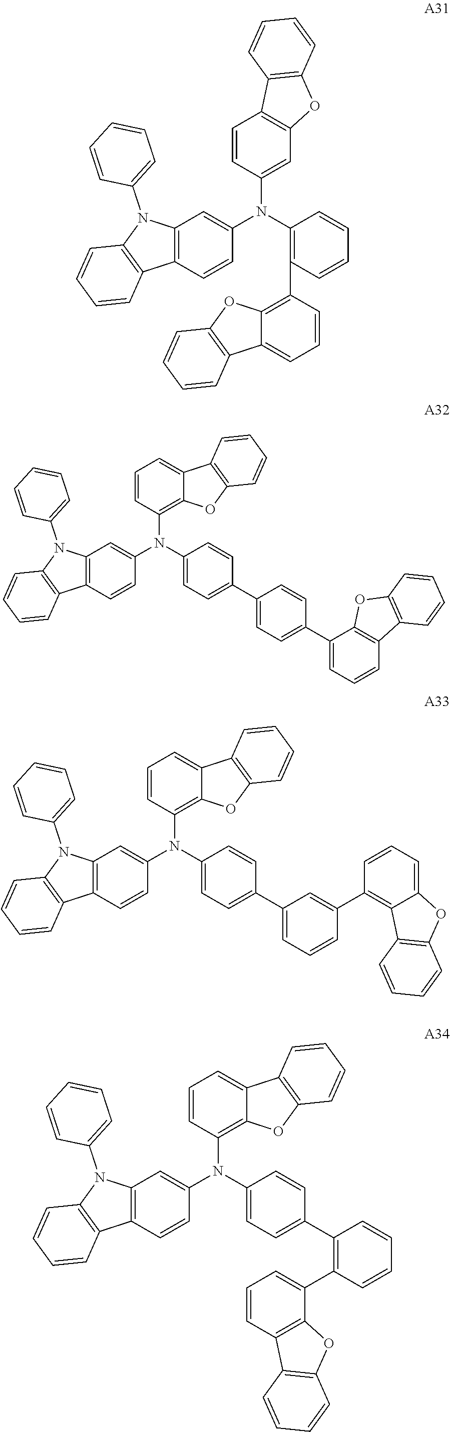 [SMILES: c1ccc(-n2c3ccccc3c3ccc(N(c4ccc5c(c4)oc4ccccc45)c4ccccc4-c4cccc5c4oc4ccccc45)cc32)cc1 ; c1ccc(-n2c3ccccc3c3ccc(N(c4ccc(-c5ccc(-c6cccc7c6oc6ccccc67)cc5)cc4)c4cccc5c4oc4ccccc45)cc32)cc1 ; c1ccc(-n2c3ccccc3c3ccc(N(c4ccc(-c5cccc(-c6cccc7oc8ccccc8c67)c5)cc4)c4cccc5c4oc4ccccc45)cc32)cc1 ; c1ccc(-n2c3ccccc3c3ccc(N(c4ccc(-c5ccccc5-c5cccc6c5oc5ccccc56)cc4)c4cccc5c4oc4ccccc45)cc32)cc1] 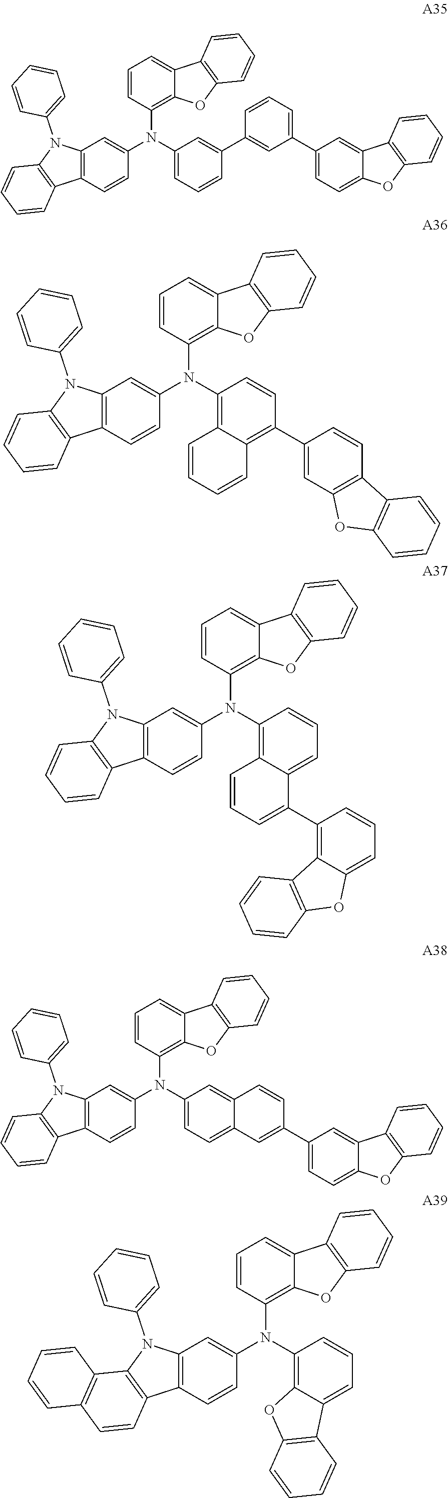 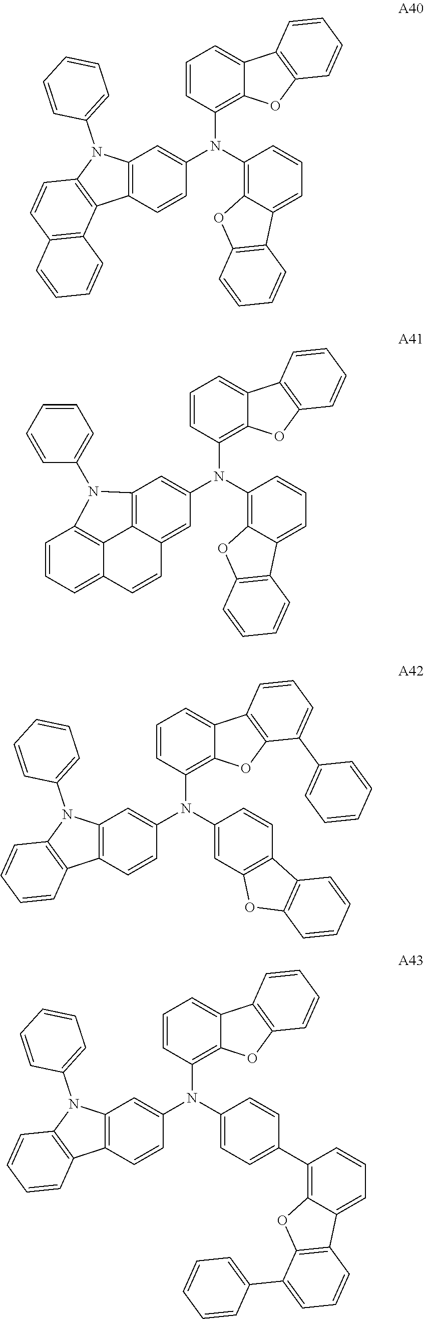 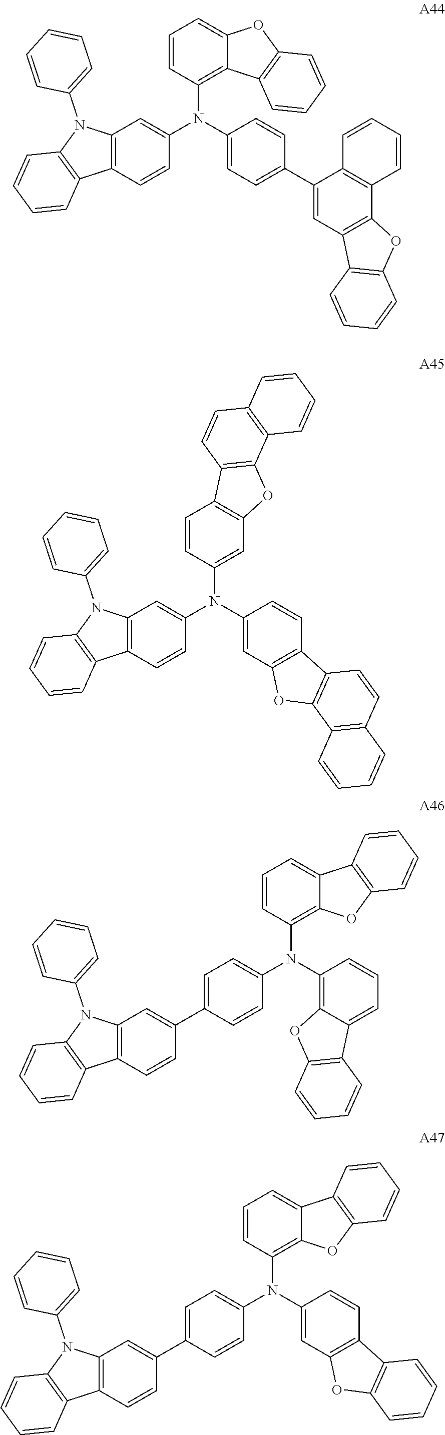 [SMILES: c1ccc(-n2c3ccccc3c3ccc(N(c4ccc(-c5cc6c7ccccc7oc6c6ccccc56)cc4)c4cccc5oc6ccccc6c45)cc32)cc1 ; c1ccc(-n2c3ccccc3c3ccc(N(c4ccc5c(c4)oc4c6ccccc6ccc54)c4ccc5c(c4)oc4c6ccccc6ccc54)cc32)cc1 ; c1ccc(-n2c3ccccc3c3ccc(-c4ccc(N(c5cccc6c5oc5ccccc56)c5cccc6c5oc5ccccc56)cc4)cc32)cc1 ; c1ccc(-n2c3ccccc3c3ccc(-c4ccc(N(c5ccc6c(c5)oc5ccccc56)c5cccc6c5oc5ccccc56)cc4)cc32)cc1] 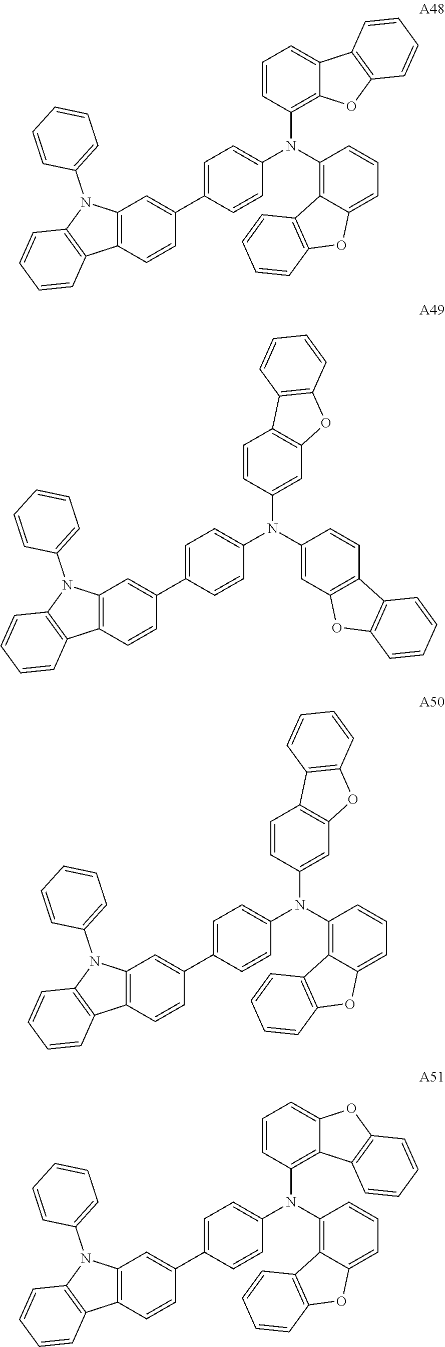 [SMILES: c1ccc(-n2c3ccccc3c3ccc(-c4ccc(N(c5cccc6c5oc5ccccc56)c5cccc6oc7ccccc7c56)cc4)cc32)cc1 ; c1ccc(-n2c3ccccc3c3ccc(-c4ccc(N(c5ccc6c(c5)oc5ccccc56)c5ccc6c(c5)oc5ccccc56)cc4)cc32)cc1 ; c1ccc(-n2c3ccccc3c3ccc(-c4ccc(N(c5ccc6c(c5)oc5ccccc56)c5cccc6oc7ccccc7c56)cc4)cc32)cc1 ; c1ccc(-n2c3ccccc3c3ccc(-c4ccc(N(c5cccc6oc7ccccc7c56)c5cccc6oc7ccccc7c56)cc4)cc32)cc1] 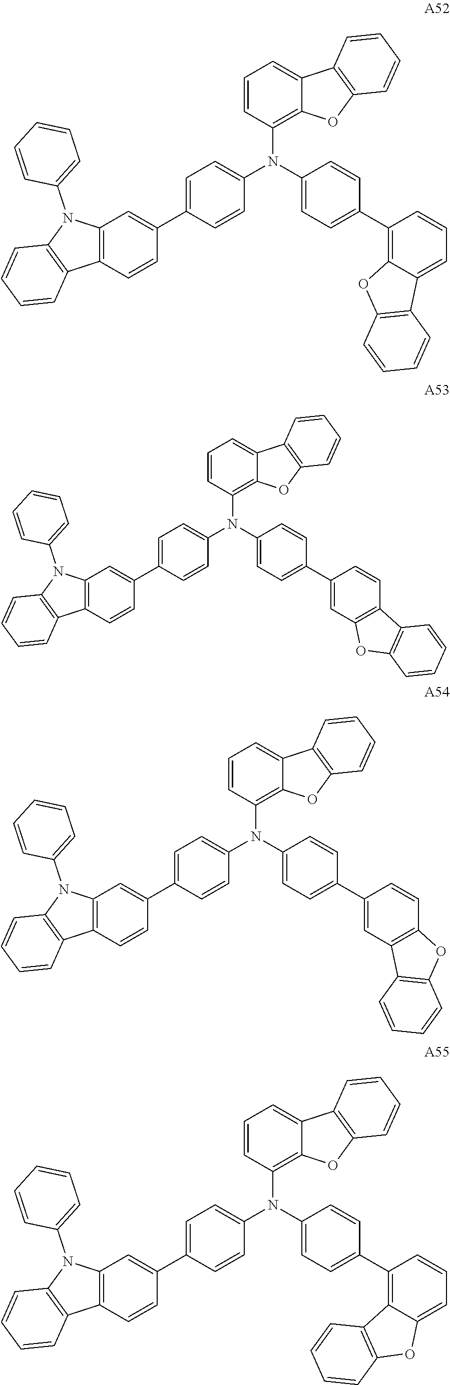 [SMILES: c1ccc(-n2c3ccccc3c3ccc(-c4ccc(N(c5ccc(-c6cccc7c6oc6ccccc67)cc5)c5cccc6c5oc5ccccc56)cc4)cc32)cc1 ; c1ccc(-n2c3ccccc3c3ccc(-c4ccc(N(c5ccc(-c6ccc7c(c6)oc6ccccc67)cc5)c5cccc6c5oc5ccccc56)cc4)cc32)cc1 ; c1ccc(-n2c3ccccc3c3ccc(-c4ccc(N(c5ccc(-c6ccc7oc8ccccc8c7c6)cc5)c5cccc6c5oc5ccccc56)cc4)cc32)cc1 ; c1ccc(-n2c3ccccc3c3ccc(-c4ccc(N(c5ccc(-c6cccc7oc8ccccc8c67)cc5)c5cccc6c5oc5ccccc56)cc4)cc32)cc1] 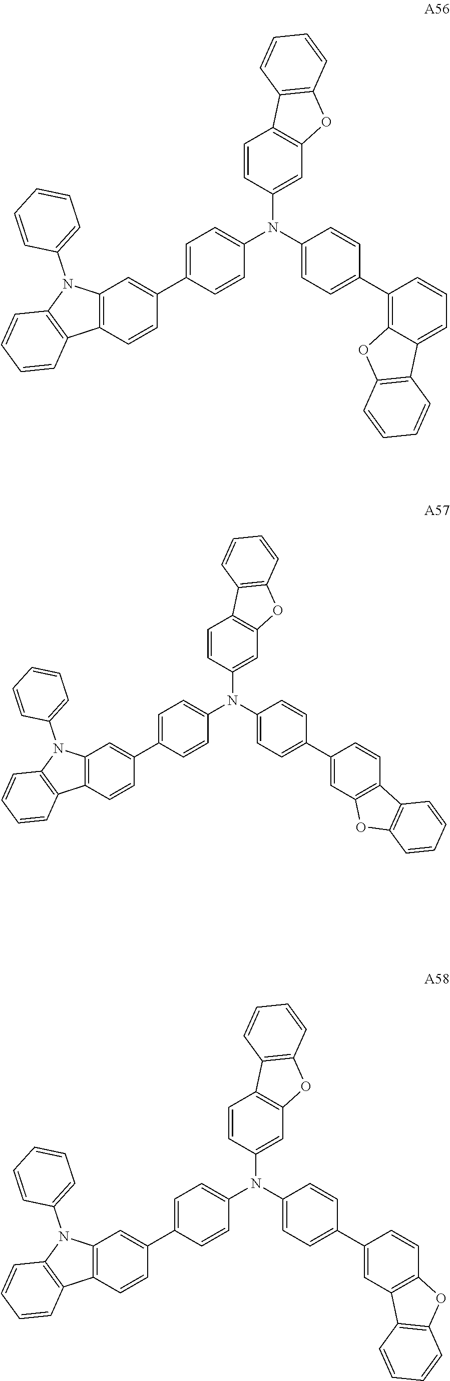 [SMILES: c1ccc(-n2c3ccccc3c3ccc(-c4ccc(N(c5ccc(-c6cccc7c6oc6ccccc67)cc5)c5ccc6c(c5)oc5ccccc56)cc4)cc32)cc1 ; c1ccc(-n2c3ccccc3c3ccc(-c4ccc(N(c5ccc(-c6ccc7c(c6)oc6ccccc67)cc5)c5ccc6c(c5)oc5ccccc56)cc4)cc32)cc1 ; c1ccc(-n2c3ccccc3c3ccc(-c4ccc(N(c5ccc(-c6ccc7oc8ccccc8c7c6)cc5)c5ccc6c(c5)oc5ccccc56)cc4)cc32)cc1] 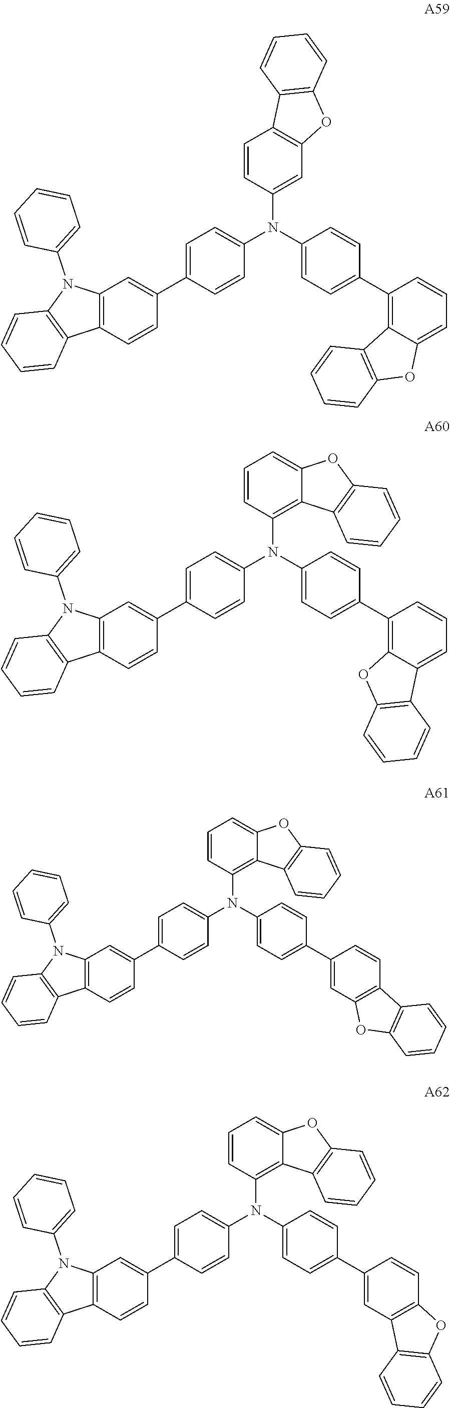 [SMILES: c1ccc(-n2c3ccccc3c3ccc(-c4ccc(N(c5ccc(-c6cccc7oc8ccccc8c67)cc5)c5ccc6c(c5)oc5ccccc56)cc4)cc32)cc1 ; c1ccc(-n2c3ccccc3c3ccc(-c4ccc(N(c5ccc(-c6cccc7c6oc6ccccc67)cc5)c5cccc6oc7ccccc7c56)cc4)cc32)cc1 ; c1ccc(-n2c3ccccc3c3ccc(-c4ccc(N(c5ccc(-c6ccc7c(c6)oc6ccccc67)cc5)c5cccc6oc7ccccc7c56)cc4)cc32)cc1 ; c1ccc(-n2c3ccccc3c3ccc(-c4ccc(N(c5ccc(-c6ccc7oc8ccccc8c7c6)cc5)c5cccc6oc7ccccc7c56)cc4)cc32)cc1] 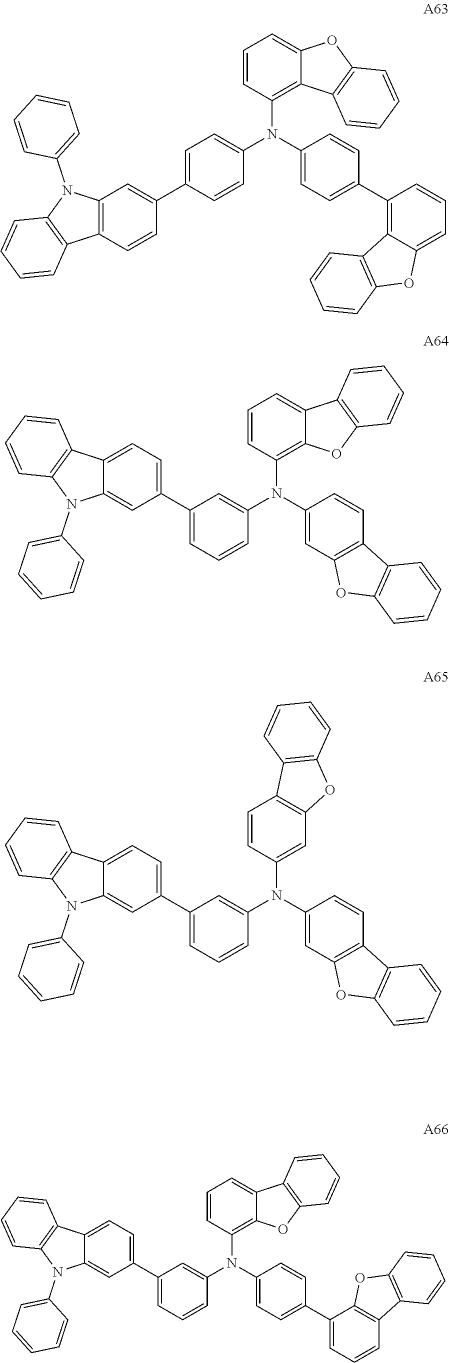 [SMILES: c1ccc(-n2c3ccccc3c3ccc(-c4ccc(N(c5ccc(-c6cccc7oc8ccccc8c67)cc5)c5cccc6oc7ccccc7c56)cc4)cc32)cc1 ; c1ccc(-n2c3ccccc3c3ccc(-c4cccc(N(c5ccc6c(c5)oc5ccccc56)c5cccc6c5oc5ccccc56)c4)cc32)cc1 ; c1ccc(-n2c3ccccc3c3ccc(-c4cccc(N(c5ccc6c(c5)oc5ccccc56)c5ccc6c(c5)oc5ccccc56)c4)cc32)cc1 ; c1ccc(-n2c3ccccc3c3ccc(-c4cccc(N(c5ccc(-c6cccc7c6oc6ccccc67)cc5)c5cccc6c5oc5ccccc56)c4)cc32)cc1] 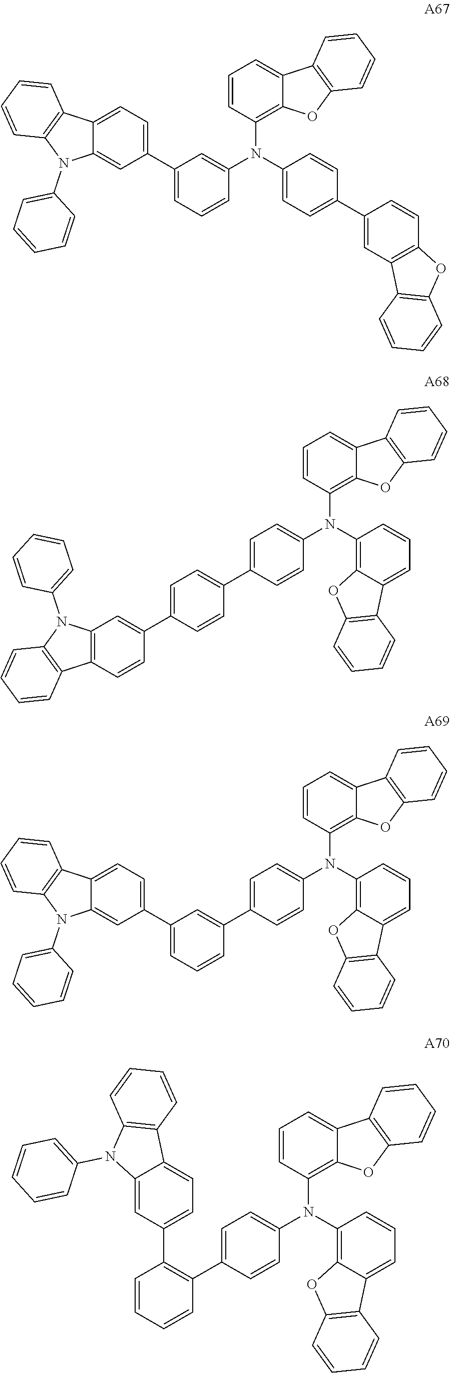 [SMILES: c1ccc(-n2c3ccccc3c3ccc(-c4cccc(N(c5ccc(-c6ccc7oc8ccccc8c7c6)cc5)c5cccc6c5oc5ccccc56)c4)cc32)cc1 ; c1ccc(-n2c3ccccc3c3ccc(-c4ccc(-c5ccc(N(c6cccc7c6oc6ccccc67)c6cccc7c6oc6ccccc67)cc5)cc4)cc32)cc1 ; c1ccc(-n2c3ccccc3c3ccc(-c4cccc(-c5ccc(N(c6cccc7c6oc6ccccc67)c6cccc7c6oc6ccccc67)cc5)c4)cc32)cc1 ; c1ccc(-n2c3ccccc3c3ccc(-c4ccccc4-c4ccc(N(c5cccc6c5oc5ccccc56)c5cccc6c5oc5ccccc56)cc4)cc32)cc1] 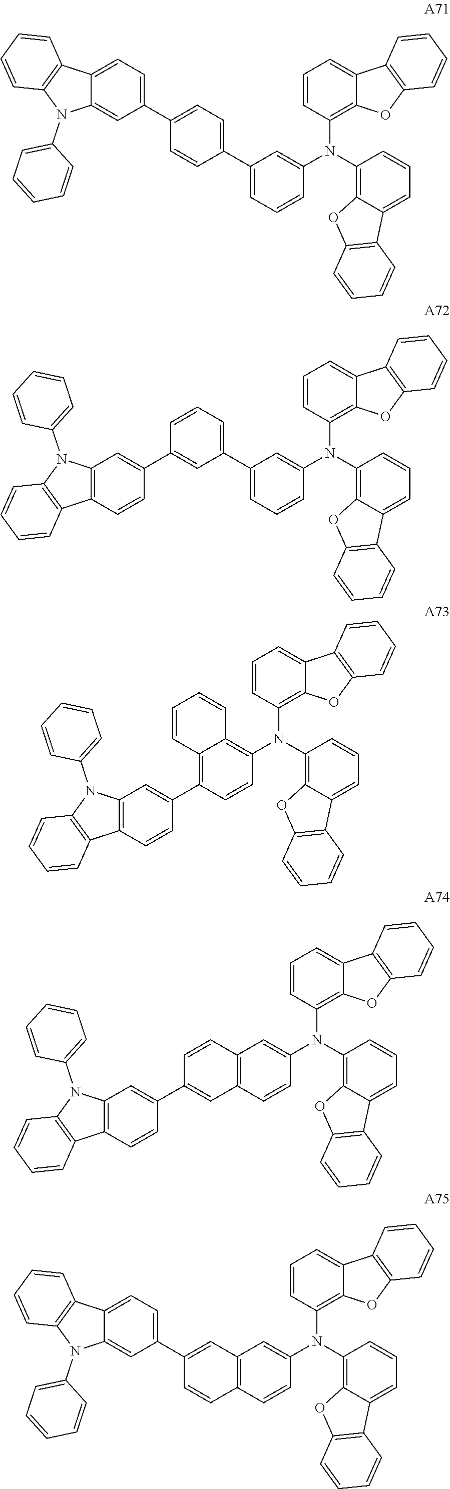 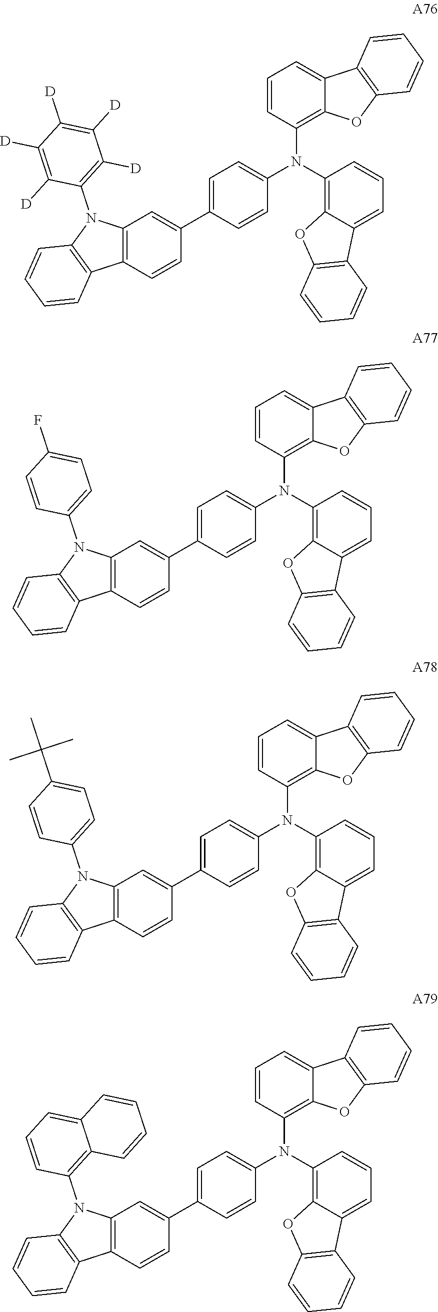 [SMILES: [2H]c1c([2H])c([2H])c(-n2c3ccccc3c3ccc(-c4ccc(N(c5cccc6c5oc5ccccc56)c5cccc6c5oc5ccccc56)cc4)cc32)c([2H])c1[2H] ; Fc1ccc(-n2c3ccccc3c3ccc(-c4ccc(N(c5cccc6c5oc5ccccc56)c5cccc6c5oc5ccccc56)cc4)cc32)cc1 ; CC(C)(C)c1ccc(-n2c3ccccc3c3ccc(-c4ccc(N(c5cccc6c5oc5ccccc56)c5cccc6c5oc5ccccc56)cc4)cc32)cc1 ; c1ccc2c(-n3c4ccccc4c4ccc(-c5ccc(N(c6cccc7c6oc6ccccc67)c6cccc7c6oc6ccccc67)cc5)cc43)cccc2c1] 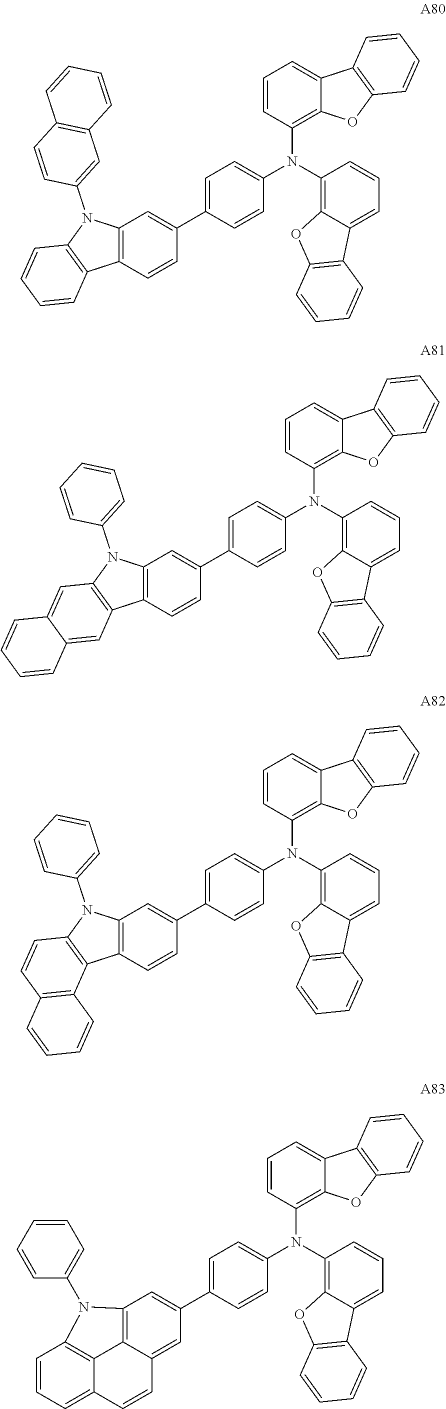 [SMILES: c1ccc2cc(-n3c4ccccc4c4ccc(-c5ccc(N(c6cccc7c6oc6ccccc67)c6cccc7c6oc6ccccc67)cc5)cc43)ccc2c1 ; c1ccc(-n2c3cc(-c4ccc(N(c5cccc6c5oc5ccccc56)c5cccc6c5oc5ccccc56)cc4)ccc3c3cc4ccccc4cc32)cc1 ; c1ccc(-n2c3cc(-c4ccc(N(c5cccc6c5oc5ccccc56)c5cccc6c5oc5ccccc56)cc4)ccc3c3c4ccccc4ccc32)cc1 ; c1ccc(-n2c3cccc4ccc5cc(-c6ccc(N(c7cccc8c7oc7ccccc78)c7cccc8c7oc7ccccc78)cc6)cc2c5c43)cc1] 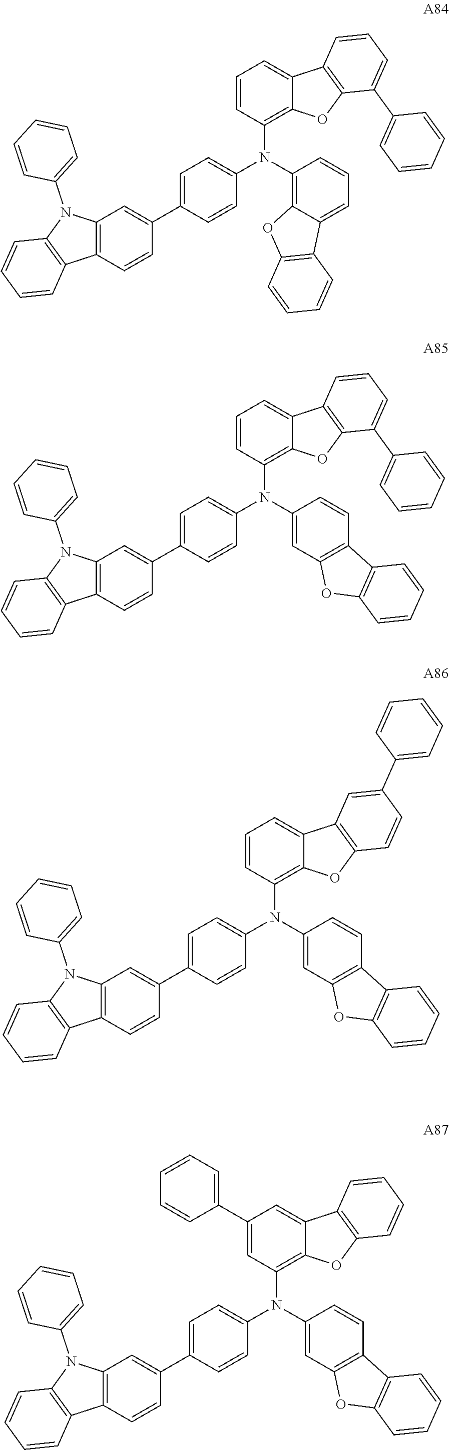 [SMILES: c1ccc(-c2cccc3c2oc2c(N(c4ccc(-c5ccc6c7ccccc7n(-c7ccccc7)c6c5)cc4)c4cccc5c4oc4ccccc45)cccc23)cc1 ; c1ccc(-c2cccc3c2oc2c(N(c4ccc(-c5ccc6c7ccccc7n(-c7ccccc7)c6c5)cc4)c4ccc5c(c4)oc4ccccc45)cccc23)cc1 ; c1ccc(-c2ccc3oc4c(N(c5ccc(-c6ccc7c8ccccc8n(-c8ccccc8)c7c6)cc5)c5ccc6c(c5)oc5ccccc56)cccc4c3c2)cc1 ; c1ccc(-c2cc(N(c3ccc(-c4ccc5c6ccccc6n(-c6ccccc6)c5c4)cc3)c3ccc4c(c3)oc3ccccc34)c3oc4ccccc4c3c2)cc1] 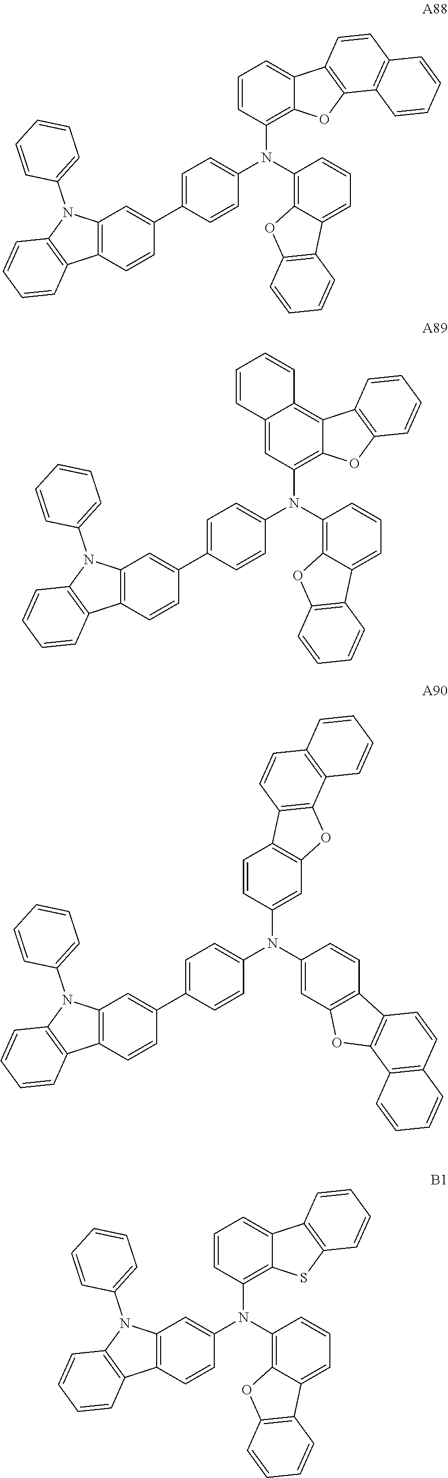 [SMILES: c1ccc(-n2c3ccccc3c3ccc(-c4ccc(N(c5cccc6c5oc5ccccc56)c5cccc6c5oc5c7ccccc7ccc65)cc4)cc32)cc1 ; c1ccc(-n2c3ccccc3c3ccc(-c4ccc(N(c5cccc6c5oc5ccccc56)c5cc6ccccc6c6c5oc5ccccc56)cc4)cc32)cc1 ; c1ccc(-n2c3ccccc3c3ccc(-c4ccc(N(c5ccc6c(c5)oc5c7ccccc7ccc65)c5ccc6c(c5)oc5c7ccccc7ccc65)cc4)cc32)cc1 ; c1ccc(-n2c3ccccc3c3ccc(N(c4cccc5c4oc4ccccc45)c4cccc5c4sc4ccccc45)cc32)cc1] 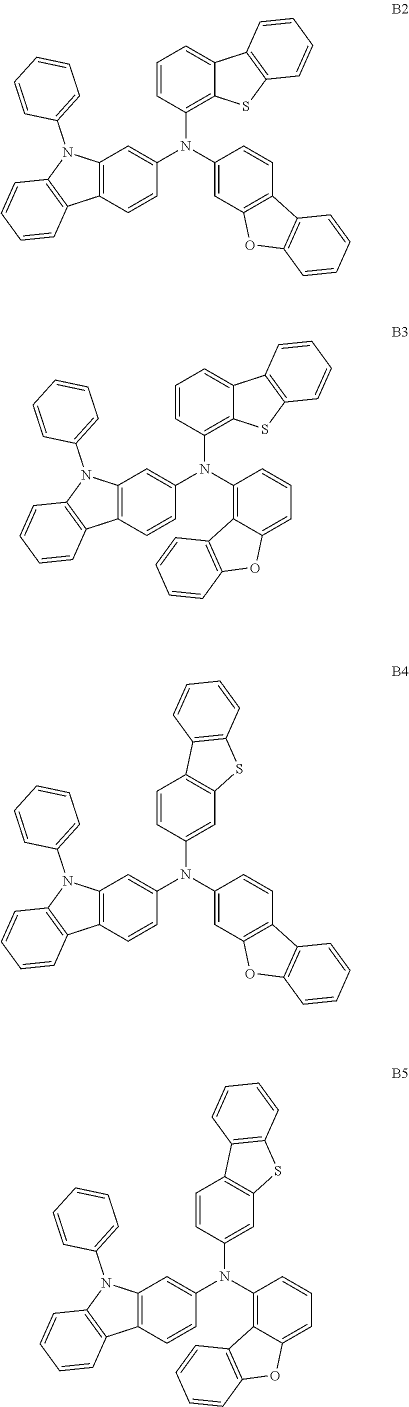 [SMILES: c1ccc(-n2c3ccccc3c3ccc(N(c4ccc5c(c4)oc4ccccc45)c4cccc5c4sc4ccccc45)cc32)cc1 ; c1ccc(-n2c3ccccc3c3ccc(N(c4cccc5c4sc4ccccc45)c4cccc5oc6ccccc6c45)cc32)cc1 ; c1ccc(-n2c3ccccc3c3ccc(N(c4ccc5c(c4)oc4ccccc45)c4ccc5c(c4)sc4ccccc45)cc32)cc1 ; c1ccc(-n2c3ccccc3c3ccc(N(c4ccc5c(c4)sc4ccccc45)c4cccc5oc6ccccc6c45)cc32)cc1] 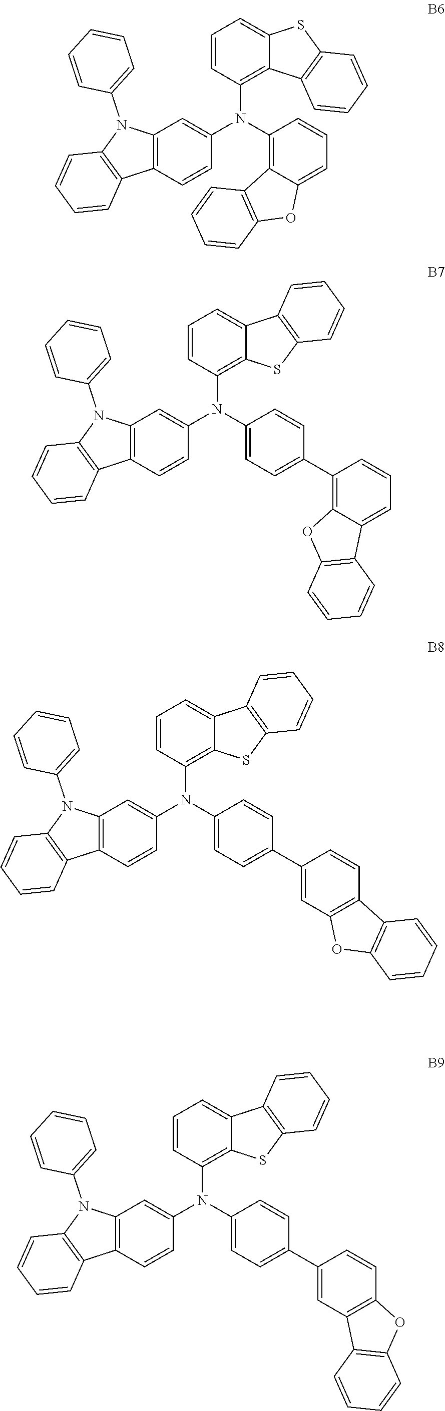 [SMILES: c1ccc(-n2c3ccccc3c3ccc(N(c4cccc5oc6ccccc6c45)c4cccc5sc6ccccc6c45)cc32)cc1 ; c1ccc(-n2c3ccccc3c3ccc(N(c4ccc(-c5cccc6c5oc5ccccc56)cc4)c4cccc5c4sc4ccccc45)cc32)cc1 ; c1ccc(-n2c3ccccc3c3ccc(N(c4ccc(-c5ccc6c(c5)oc5ccccc56)cc4)c4cccc5c4sc4ccccc45)cc32)cc1 ; c1ccc(-n2c3ccccc3c3ccc(N(c4ccc(-c5ccc6oc7ccccc7c6c5)cc4)c4cccc5c4sc4ccccc45)cc32)cc1] 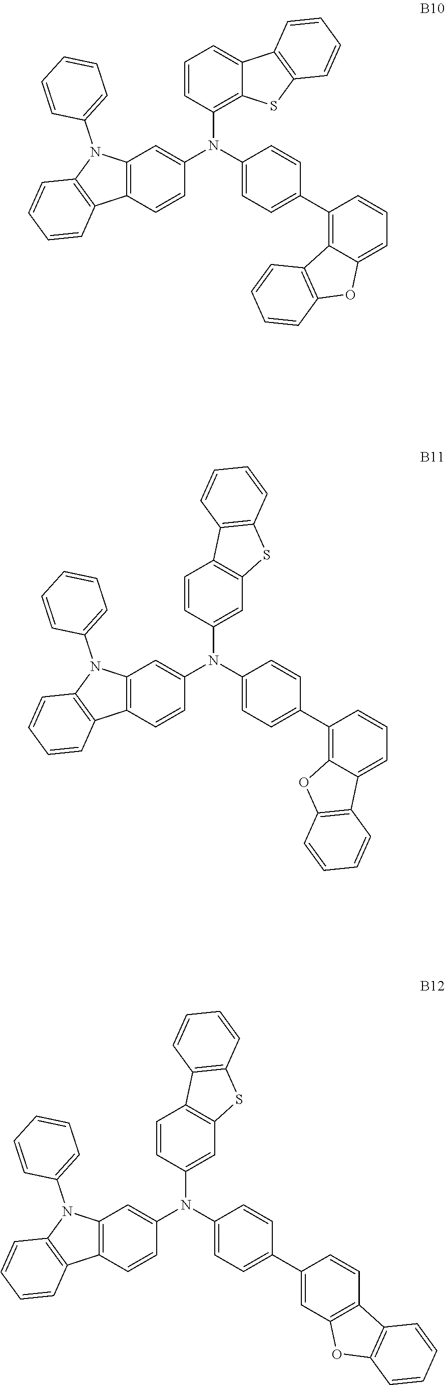 [SMILES: c1ccc(-n2c3ccccc3c3ccc(N(c4ccc(-c5cccc6oc7ccccc7c56)cc4)c4cccc5c4sc4ccccc45)cc32)cc1 ; c1ccc(-n2c3ccccc3c3ccc(N(c4ccc(-c5cccc6c5oc5ccccc56)cc4)c4ccc5c(c4)sc4ccccc45)cc32)cc1 ; c1ccc(-n2c3ccccc3c3ccc(N(c4ccc(-c5ccc6c(c5)oc5ccccc56)cc4)c4ccc5c(c4)sc4ccccc45)cc32)cc1] 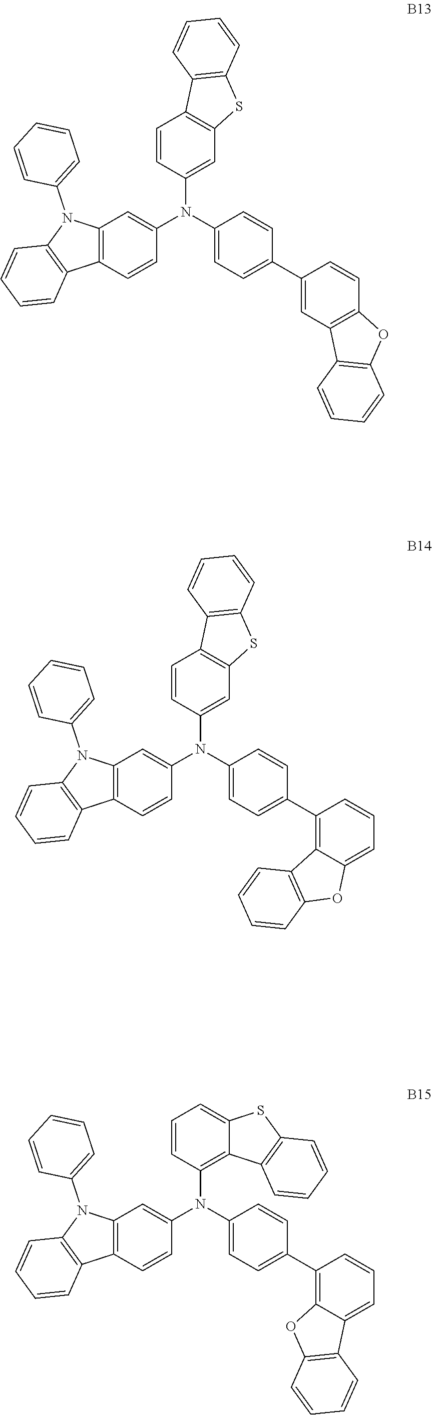 [SMILES: c1ccc(-n2c3ccccc3c3ccc(N(c4ccc(-c5ccc6oc7ccccc7c6c5)cc4)c4ccc5c(c4)sc4ccccc45)cc32)cc1 ; c1ccc(-n2c3ccccc3c3ccc(N(c4ccc(-c5cccc6oc7ccccc7c56)cc4)c4ccc5c(c4)sc4ccccc45)cc32)cc1 ; c1ccc(-n2c3ccccc3c3ccc(N(c4ccc(-c5cccc6c5oc5ccccc56)cc4)c4cccc5sc6ccccc6c45)cc32)cc1] 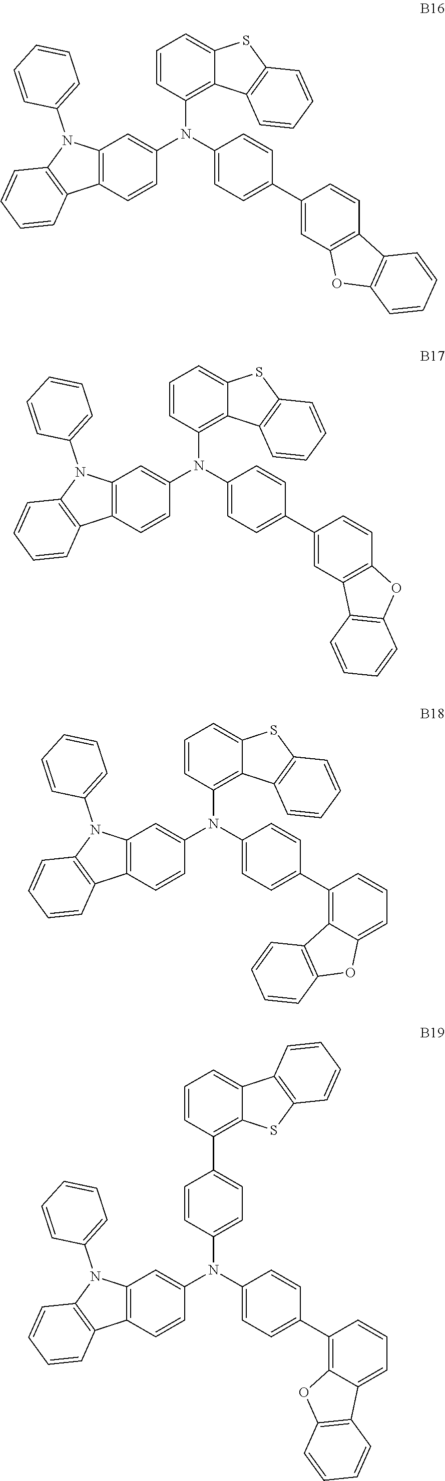 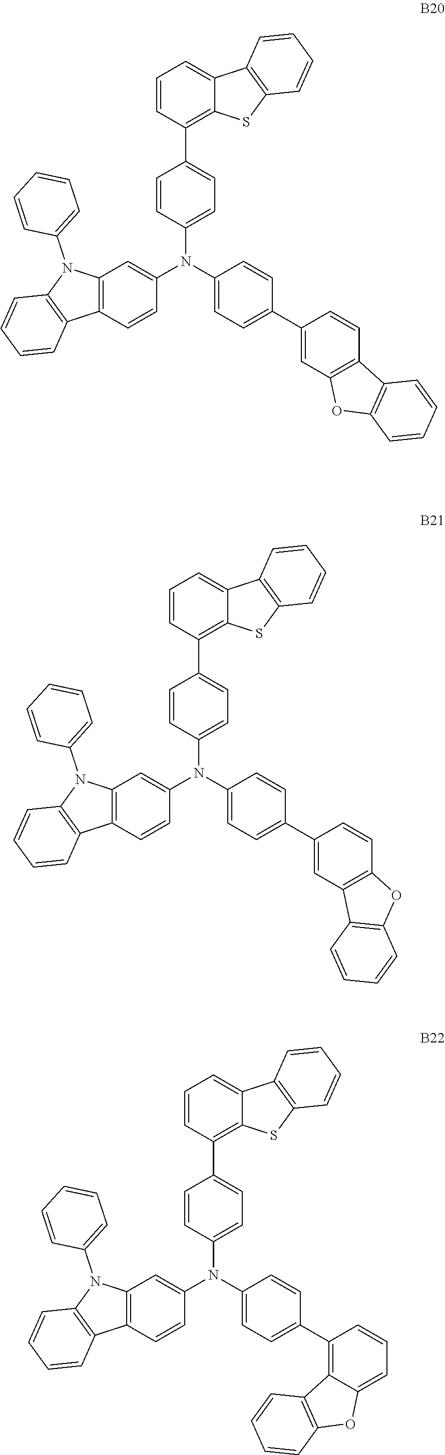 [SMILES: c1ccc(-n2c3ccccc3c3ccc(N(c4ccc(-c5ccc6c(c5)oc5ccccc56)cc4)c4ccc(-c5cccc6c5sc5ccccc56)cc4)cc32)cc1 ; c1ccc(-n2c3ccccc3c3ccc(N(c4ccc(-c5ccc6oc7ccccc7c6c5)cc4)c4ccc(-c5cccc6c5sc5ccccc56)cc4)cc32)cc1 ; c1ccc(-n2c3ccccc3c3ccc(N(c4ccc(-c5cccc6c5sc5ccccc56)cc4)c4ccc(-c5cccc6oc7ccccc7c56)cc4)cc32)cc1] 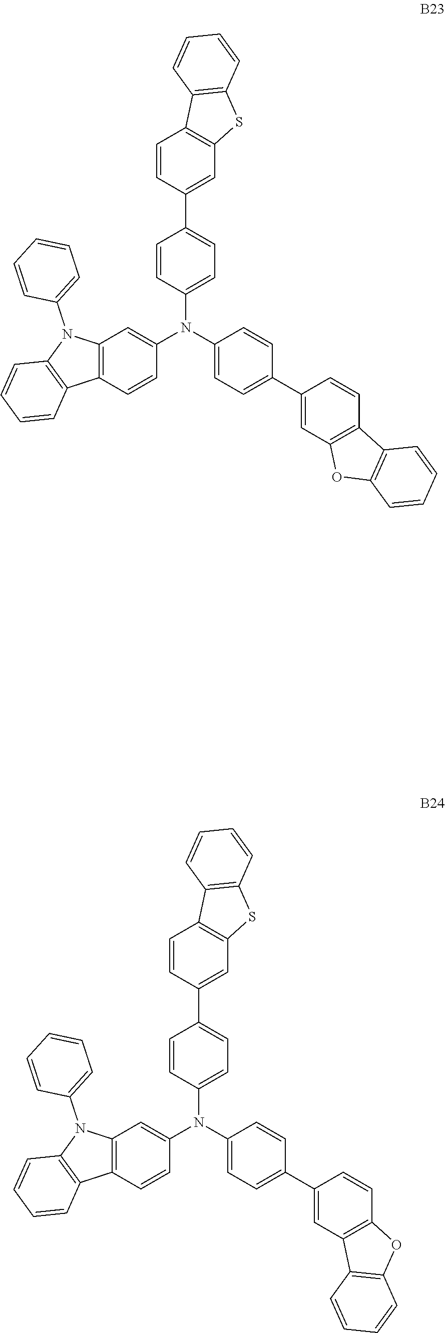 [SMILES: c1ccc(-n2c3ccccc3c3ccc(N(c4ccc(-c5ccc6c(c5)oc5ccccc56)cc4)c4ccc(-c5ccc6c(c5)sc5ccccc56)cc4)cc32)cc1 ; c1ccc(-n2c3ccccc3c3ccc(N(c4ccc(-c5ccc6c(c5)sc5ccccc56)cc4)c4ccc(-c5ccc6oc7ccccc7c6c5)cc4)cc32)cc1] 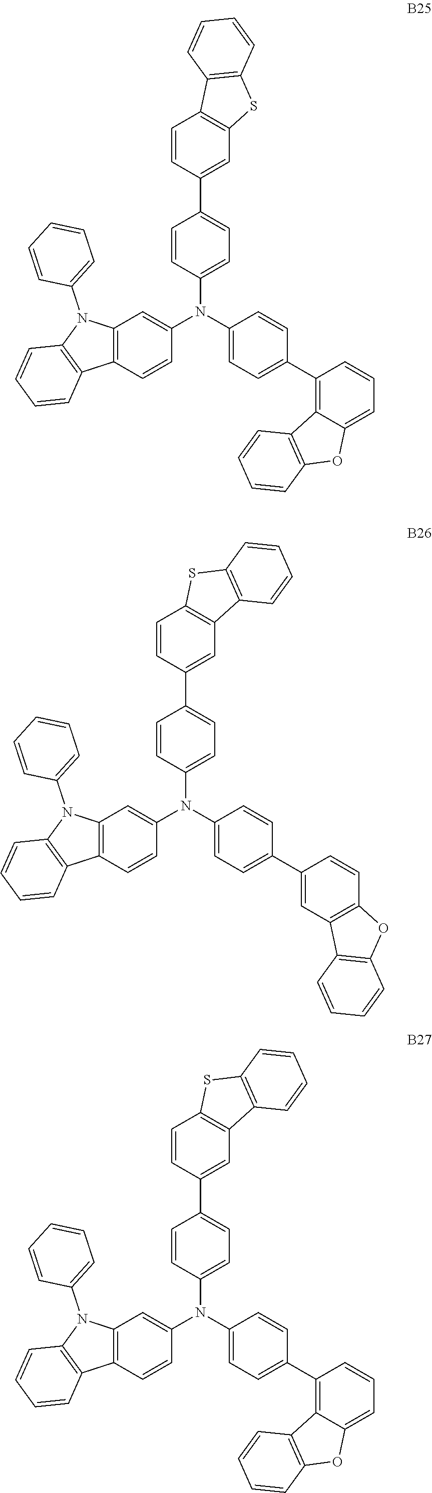 [SMILES: c1ccc(-n2c3ccccc3c3ccc(N(c4ccc(-c5ccc6c(c5)sc5ccccc56)cc4)c4ccc(-c5cccc6oc7ccccc7c56)cc4)cc32)cc1 ; c1ccc(-n2c3ccccc3c3ccc(N(c4ccc(-c5ccc6oc7ccccc7c6c5)cc4)c4ccc(-c5ccc6sc7ccccc7c6c5)cc4)cc32)cc1 ; c1ccc(-n2c3ccccc3c3ccc(N(c4ccc(-c5ccc6sc7ccccc7c6c5)cc4)c4ccc(-c5cccc6oc7ccccc7c56)cc4)cc32)cc1] 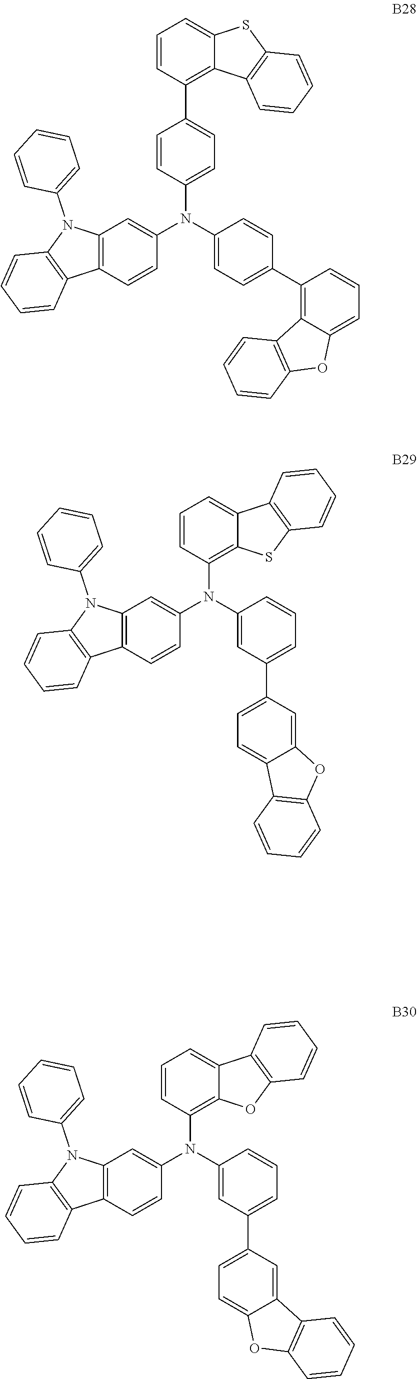 [SMILES: c1ccc(-n2c3ccccc3c3ccc(N(c4ccc(-c5cccc6oc7ccccc7c56)cc4)c4ccc(-c5cccc6sc7ccccc7c56)cc4)cc32)cc1 ; c1ccc(-n2c3ccccc3c3ccc(N(c4cccc(-c5ccc6c(c5)oc5ccccc56)c4)c4cccc5c4sc4ccccc45)cc32)cc1 ; c1ccc(-n2c3ccccc3c3ccc(N(c4cccc(-c5ccc6oc7ccccc7c6c5)c4)c4cccc5c4oc4ccccc45)cc32)cc1] 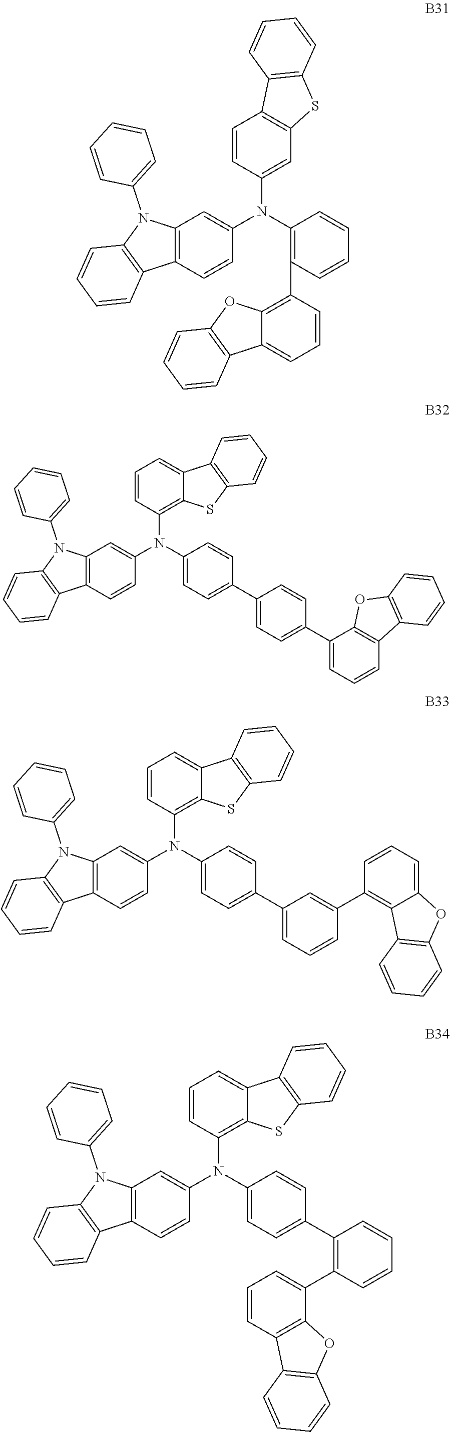 [SMILES: c1ccc(-n2c3ccccc3c3ccc(N(c4ccc5c(c4)sc4ccccc45)c4ccccc4-c4cccc5c4oc4ccccc45)cc32)cc1 ; c1ccc(-n2c3ccccc3c3ccc(N(c4ccc(-c5ccc(-c6cccc7c6oc6ccccc67)cc5)cc4)c4cccc5c4sc4ccccc45)cc32)cc1 ; c1ccc(-n2c3ccccc3c3ccc(N(c4ccc(-c5cccc(-c6cccc7oc8ccccc8c67)c5)cc4)c4cccc5c4sc4ccccc45)cc32)cc1 ; c1ccc(-n2c3ccccc3c3ccc(N(c4ccc(-c5ccccc5-c5cccc6c5oc5ccccc56)cc4)c4cccc5c4sc4ccccc45)cc32)cc1] 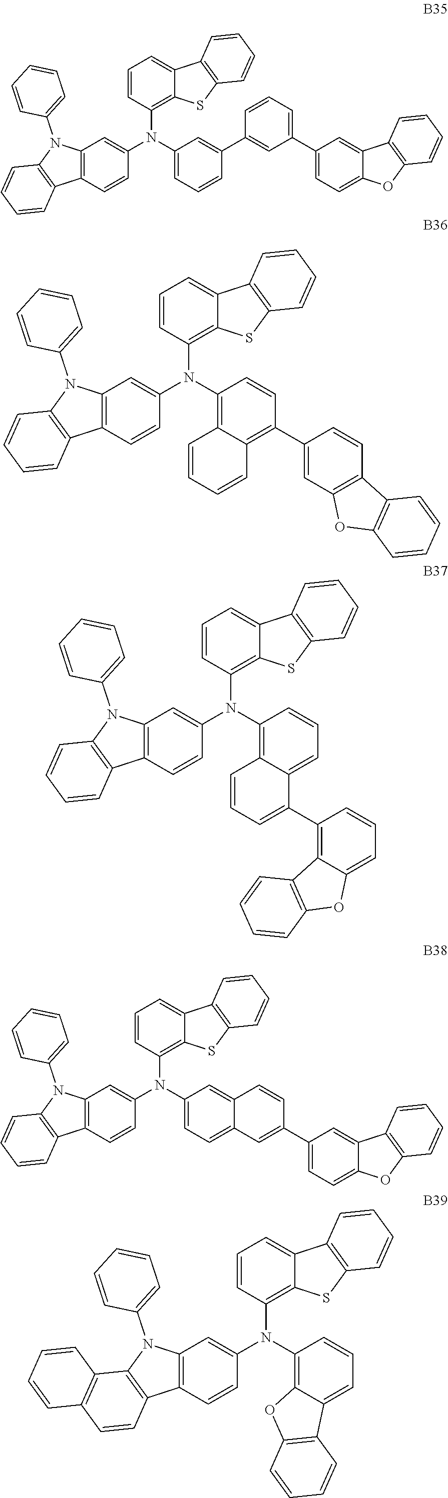 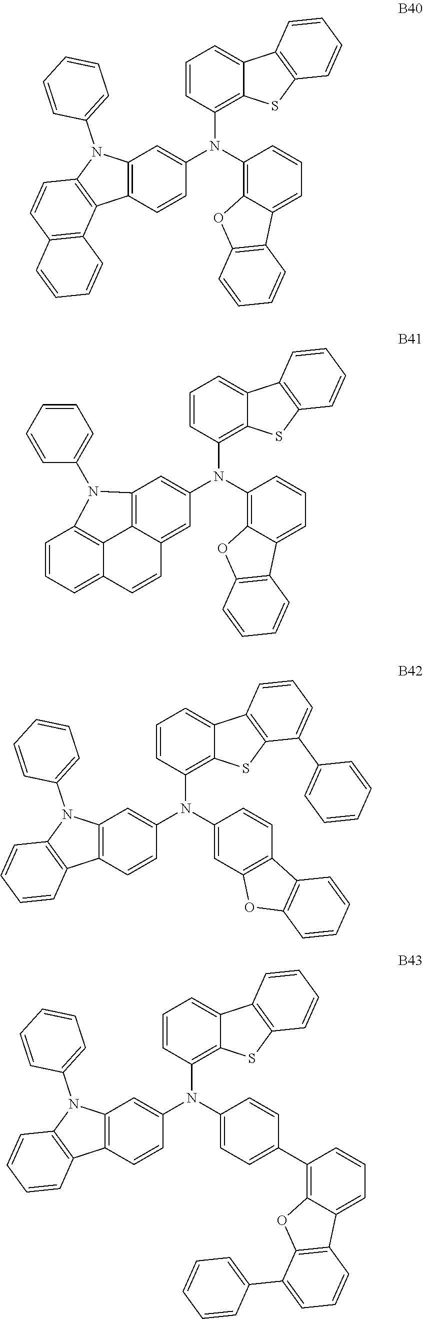 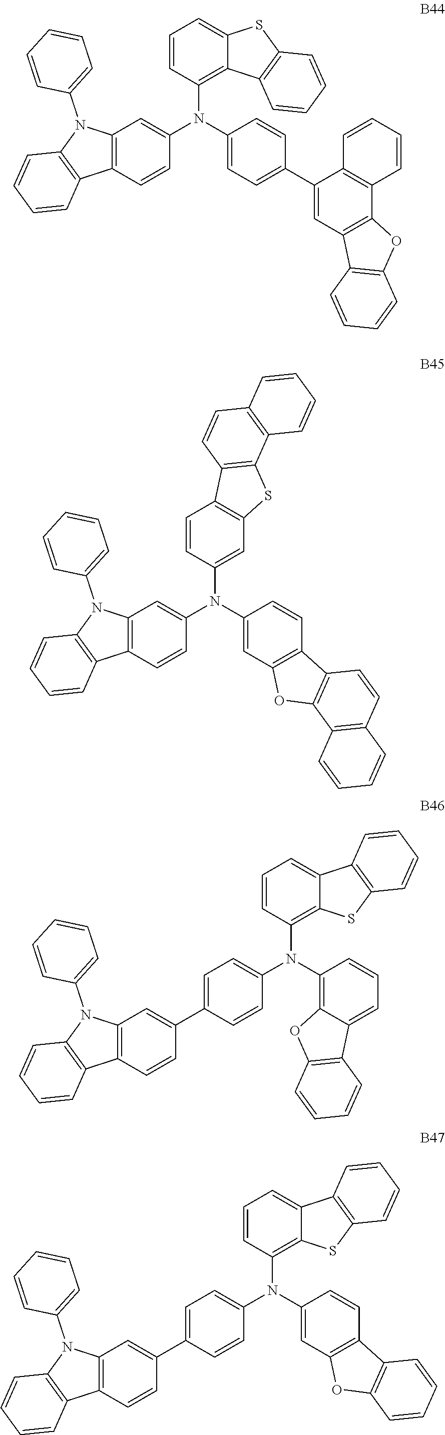 [SMILES: c1ccc(-n2c3ccccc3c3ccc(N(c4ccc(-c5cc6c7ccccc7oc6c6ccccc56)cc4)c4cccc5sc6ccccc6c45)cc32)cc1 ; c1ccc(-n2c3ccccc3c3ccc(N(c4ccc5c(c4)oc4c6ccccc6ccc54)c4ccc5c(c4)sc4c6ccccc6ccc54)cc32)cc1 ; c1ccc(-n2c3ccccc3c3ccc(-c4ccc(N(c5cccc6c5oc5ccccc56)c5cccc6c5sc5ccccc56)cc4)cc32)cc1 ; c1ccc(-n2c3ccccc3c3ccc(-c4ccc(N(c5ccc6c(c5)oc5ccccc56)c5cccc6c5sc5ccccc56)cc4)cc32)cc1] 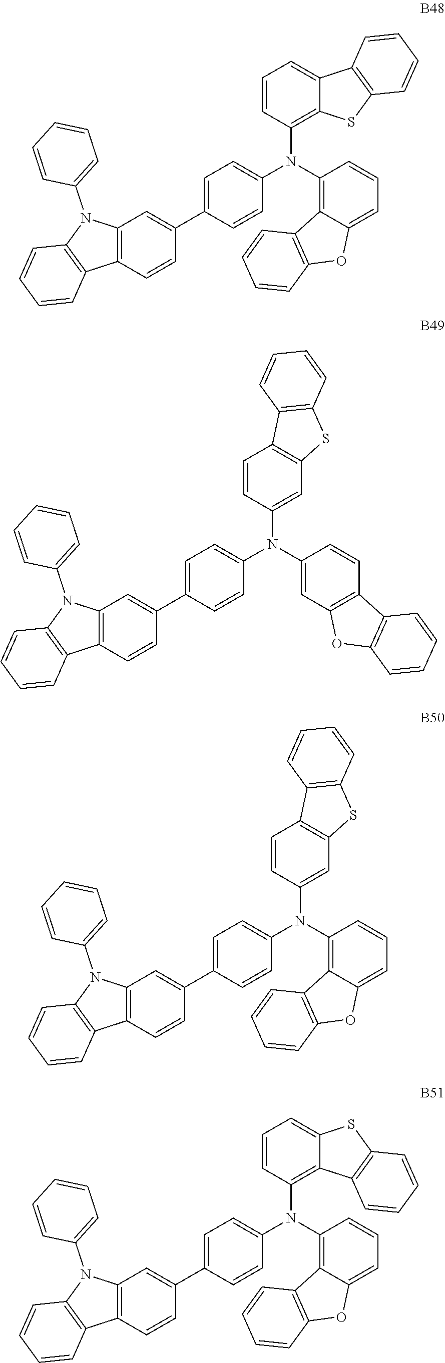 [SMILES: c1ccc(-n2c3ccccc3c3ccc(-c4ccc(N(c5cccc6c5sc5ccccc56)c5cccc6oc7ccccc7c56)cc4)cc32)cc1 ; c1ccc(-n2c3ccccc3c3ccc(-c4ccc(N(c5ccc6c(c5)oc5ccccc56)c5ccc6c(c5)sc5ccccc56)cc4)cc32)cc1 ; c1ccc(-n2c3ccccc3c3ccc(-c4ccc(N(c5ccc6c(c5)sc5ccccc56)c5cccc6oc7ccccc7c56)cc4)cc32)cc1 ; c1ccc(-n2c3ccccc3c3ccc(-c4ccc(N(c5cccc6oc7ccccc7c56)c5cccc6sc7ccccc7c56)cc4)cc32)cc1] 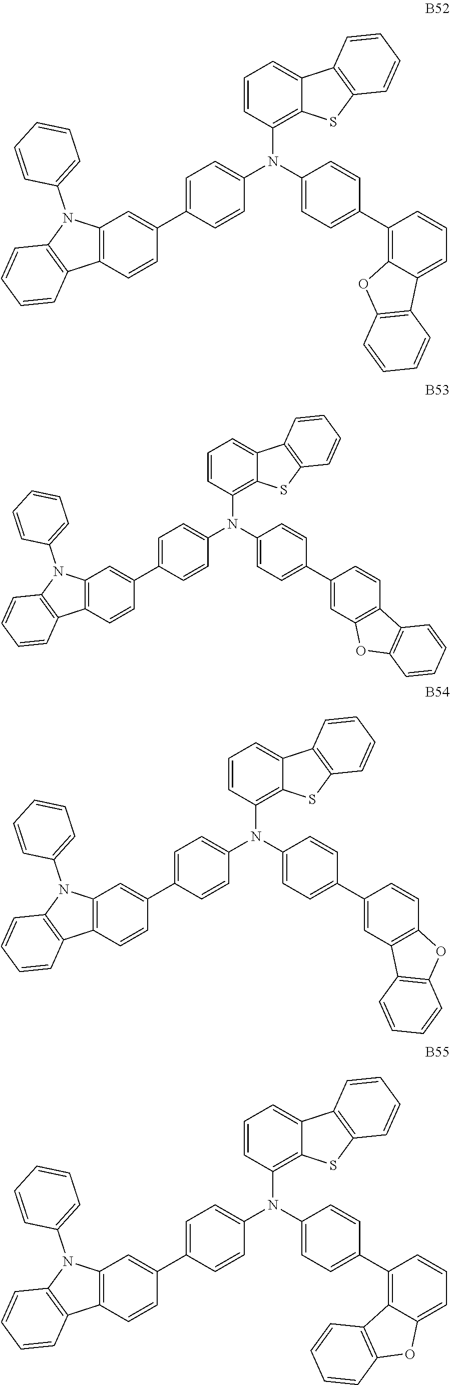 [SMILES: c1ccc(-n2c3ccccc3c3ccc(-c4ccc(N(c5ccc(-c6cccc7c6oc6ccccc67)cc5)c5cccc6c5sc5ccccc56)cc4)cc32)cc1 ; c1ccc(-n2c3ccccc3c3ccc(-c4ccc(N(c5ccc(-c6ccc7c(c6)oc6ccccc67)cc5)c5cccc6c5sc5ccccc56)cc4)cc32)cc1 ; c1ccc(-n2c3ccccc3c3ccc(-c4ccc(N(c5ccc(-c6ccc7oc8ccccc8c7c6)cc5)c5cccc6c5sc5ccccc56)cc4)cc32)cc1 ; c1ccc(-n2c3ccccc3c3ccc(-c4ccc(N(c5ccc(-c6cccc7oc8ccccc8c67)cc5)c5cccc6c5sc5ccccc56)cc4)cc32)cc1] 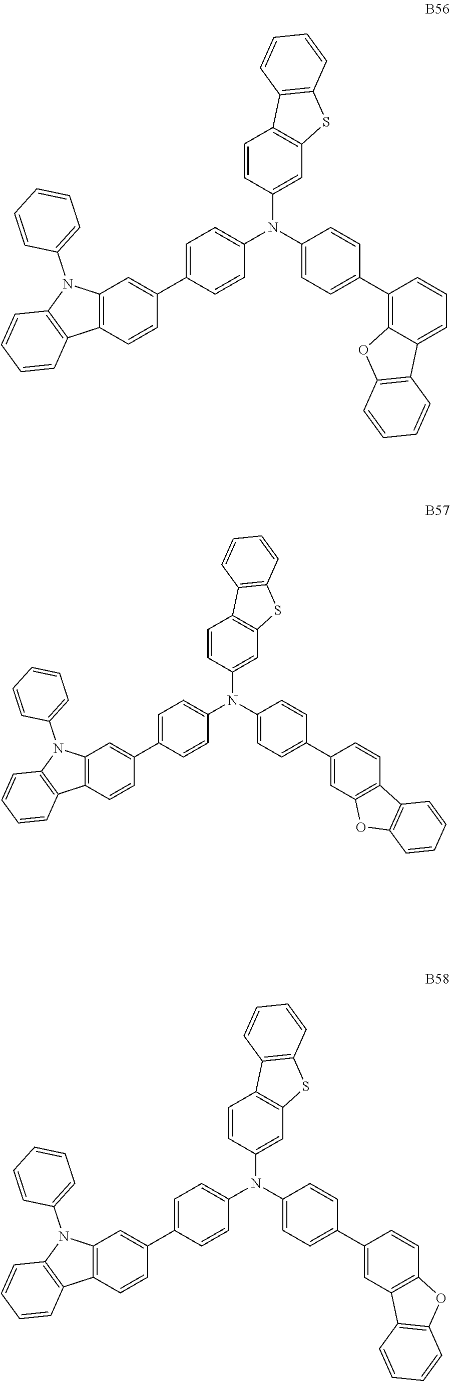 [SMILES: c1ccc(-n2c3ccccc3c3ccc(-c4ccc(N(c5ccc(-c6cccc7c6oc6ccccc67)cc5)c5ccc6c(c5)sc5ccccc56)cc4)cc32)cc1 ; c1ccc(-n2c3ccccc3c3ccc(-c4ccc(N(c5ccc(-c6ccc7c(c6)oc6ccccc67)cc5)c5ccc6c(c5)sc5ccccc56)cc4)cc32)cc1 ; c1ccc(-n2c3ccccc3c3ccc(-c4ccc(N(c5ccc(-c6ccc7oc8ccccc8c7c6)cc5)c5ccc6c(c5)sc5ccccc56)cc4)cc32)cc1] 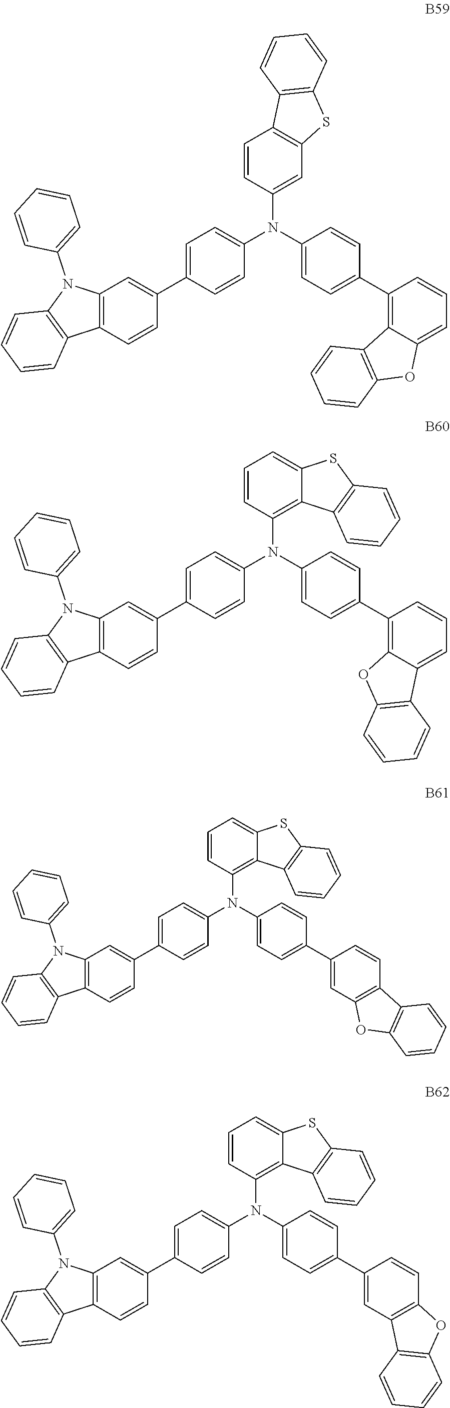 [SMILES: c1ccc(-n2c3ccccc3c3ccc(-c4ccc(N(c5ccc(-c6cccc7oc8ccccc8c67)cc5)c5ccc6c(c5)sc5ccccc56)cc4)cc32)cc1 ; c1ccc(-n2c3ccccc3c3ccc(-c4ccc(N(c5ccc(-c6cccc7c6oc6ccccc67)cc5)c5cccc6sc7ccccc7c56)cc4)cc32)cc1 ; c1ccc(-n2c3ccccc3c3ccc(-c4ccc(N(c5ccc(-c6ccc7c(c6)oc6ccccc67)cc5)c5cccc6sc7ccccc7c56)cc4)cc32)cc1 ; c1ccc(-n2c3ccccc3c3ccc(-c4ccc(N(c5ccc(-c6ccc7oc8ccccc8c7c6)cc5)c5cccc6sc7ccccc7c56)cc4)cc32)cc1] 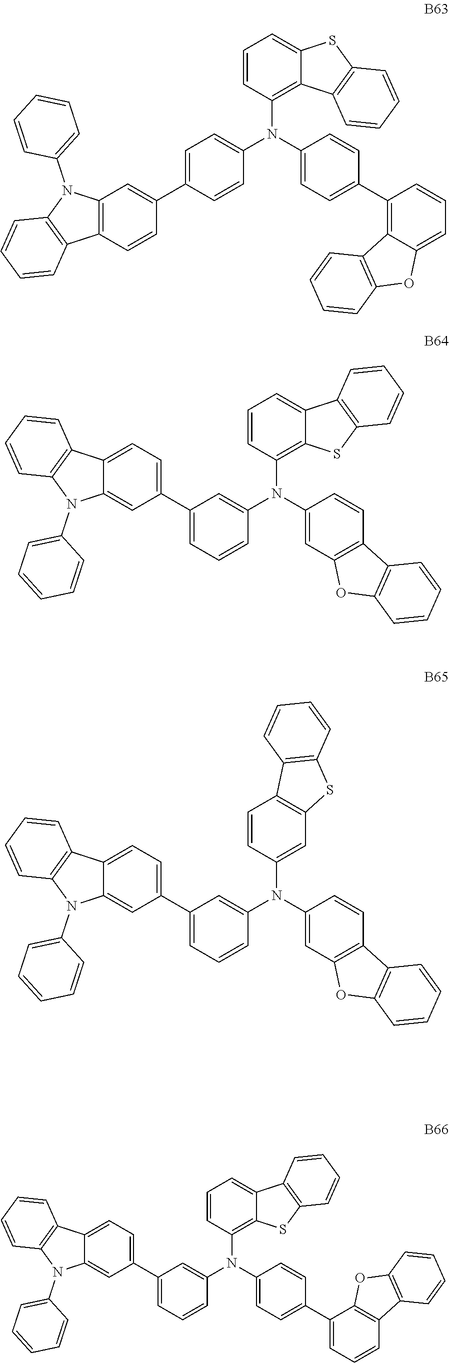 [SMILES: c1ccc(-n2c3ccccc3c3ccc(-c4ccc(N(c5ccc(-c6cccc7oc8ccccc8c67)cc5)c5cccc6sc7ccccc7c56)cc4)cc32)cc1 ; c1ccc(-n2c3ccccc3c3ccc(-c4cccc(N(c5ccc6c(c5)oc5ccccc56)c5cccc6c5sc5ccccc56)c4)cc32)cc1 ; c1ccc(-n2c3ccccc3c3ccc(-c4cccc(N(c5ccc6c(c5)oc5ccccc56)c5ccc6c(c5)sc5ccccc56)c4)cc32)cc1 ; c1ccc(-n2c3ccccc3c3ccc(-c4cccc(N(c5ccc(-c6cccc7c6oc6ccccc67)cc5)c5cccc6c5sc5ccccc56)c4)cc32)cc1] 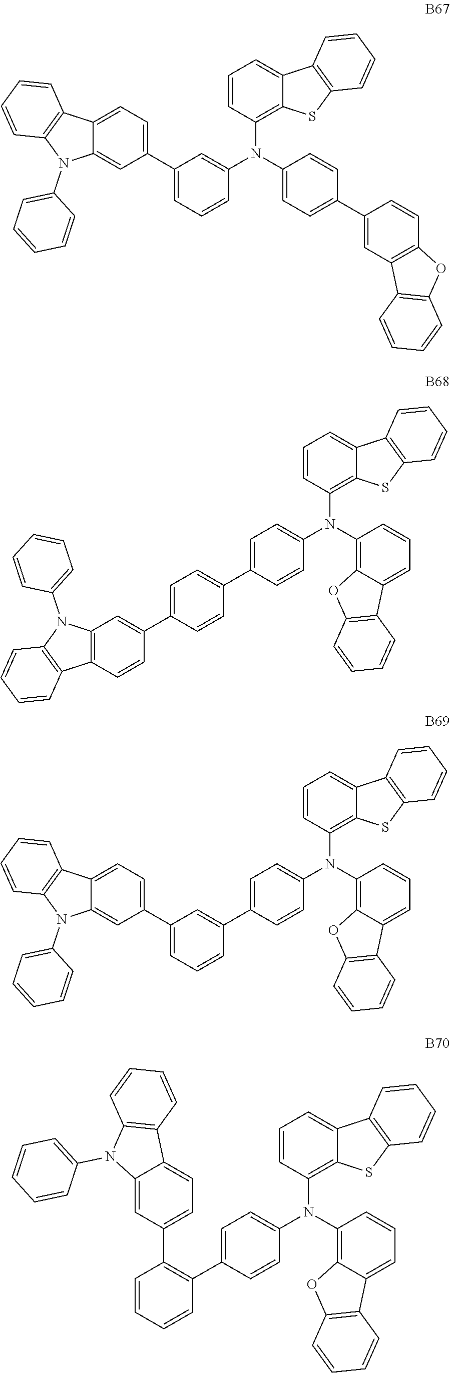 [SMILES: c1ccc(-n2c3ccccc3c3ccc(-c4cccc(N(c5ccc(-c6ccc7oc8ccccc8c7c6)cc5)c5cccc6c5sc5ccccc56)c4)cc32)cc1 ; c1ccc(-n2c3ccccc3c3ccc(-c4ccc(-c5ccc(N(c6cccc7c6oc6ccccc67)c6cccc7c6sc6ccccc67)cc5)cc4)cc32)cc1 ; c1ccc(-n2c3ccccc3c3ccc(-c4cccc(-c5ccc(N(c6cccc7c6oc6ccccc67)c6cccc7c6sc6ccccc67)cc5)c4)cc32)cc1 ; c1ccc(-n2c3ccccc3c3ccc(-c4ccccc4-c4ccc(N(c5cccc6c5oc5ccccc56)c5cccc6c5sc5ccccc56)cc4)cc32)cc1] 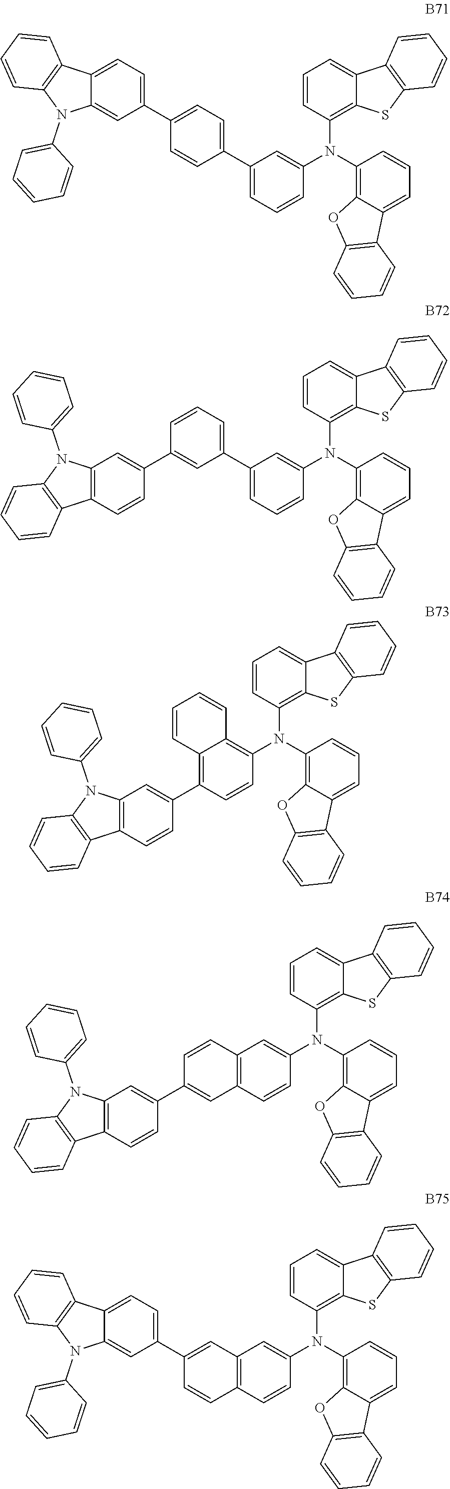 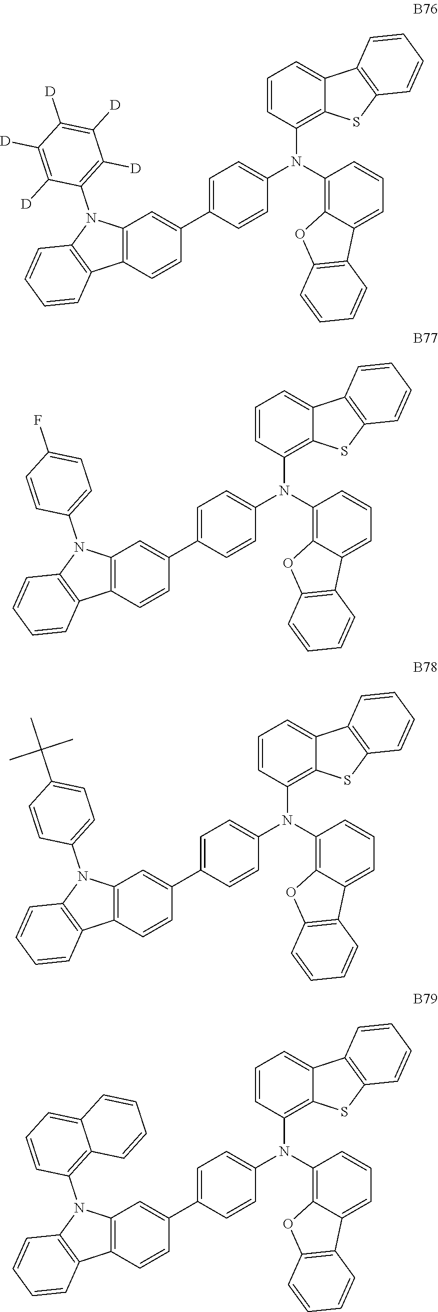 [SMILES: [2H]c1c([2H])c([2H])c(-n2c3ccccc3c3ccc(-c4ccc(N(c5cccc6c5oc5ccccc56)c5cccc6c5sc5ccccc56)cc4)cc32)c([2H])c1[2H] ; Fc1ccc(-n2c3ccccc3c3ccc(-c4ccc(N(c5cccc6c5oc5ccccc56)c5cccc6c5sc5ccccc56)cc4)cc32)cc1 ; CC(C)(C)c1ccc(-n2c3ccccc3c3ccc(-c4ccc(N(c5cccc6c5oc5ccccc56)c5cccc6c5sc5ccccc56)cc4)cc32)cc1 ; c1ccc2c(-n3c4ccccc4c4ccc(-c5ccc(N(c6cccc7c6oc6ccccc67)c6cccc7c6sc6ccccc67)cc5)cc43)cccc2c1] 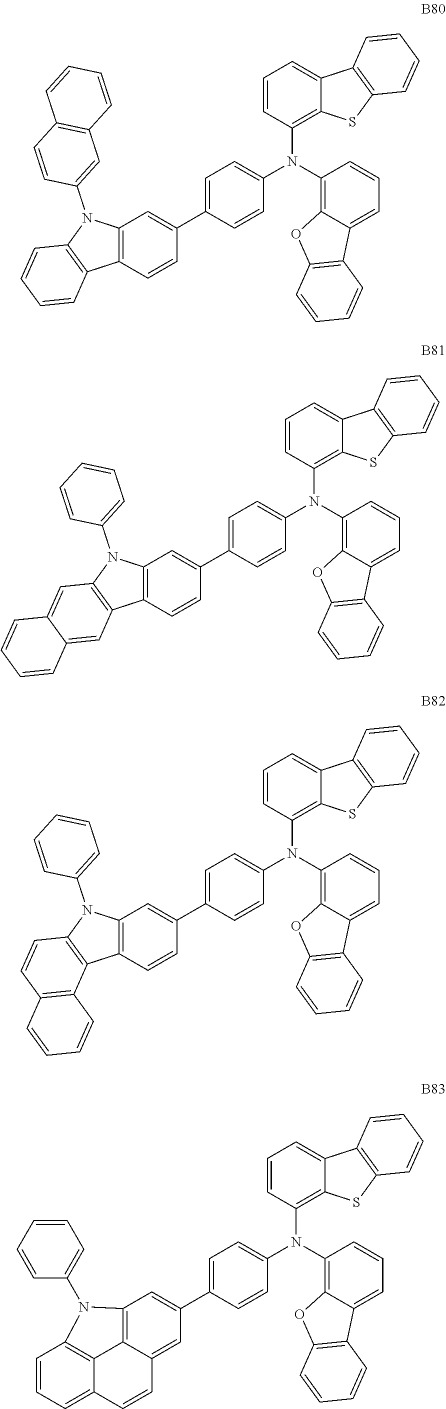 [SMILES: c1ccc2cc(-n3c4ccccc4c4ccc(-c5ccc(N(c6cccc7c6oc6ccccc67)c6cccc7c6sc6ccccc67)cc5)cc43)ccc2c1 ; c1ccc(-n2c3cc(-c4ccc(N(c5cccc6c5oc5ccccc56)c5cccc6c5sc5ccccc56)cc4)ccc3c3cc4ccccc4cc32)cc1 ; c1ccc(-n2c3cc(-c4ccc(N(c5cccc6c5oc5ccccc56)c5cccc6c5sc5ccccc56)cc4)ccc3c3c4ccccc4ccc32)cc1 ; c1ccc(-n2c3cccc4ccc5cc(-c6ccc(N(c7cccc8c7oc7ccccc78)c7cccc8c7sc7ccccc78)cc6)cc2c5c43)cc1] 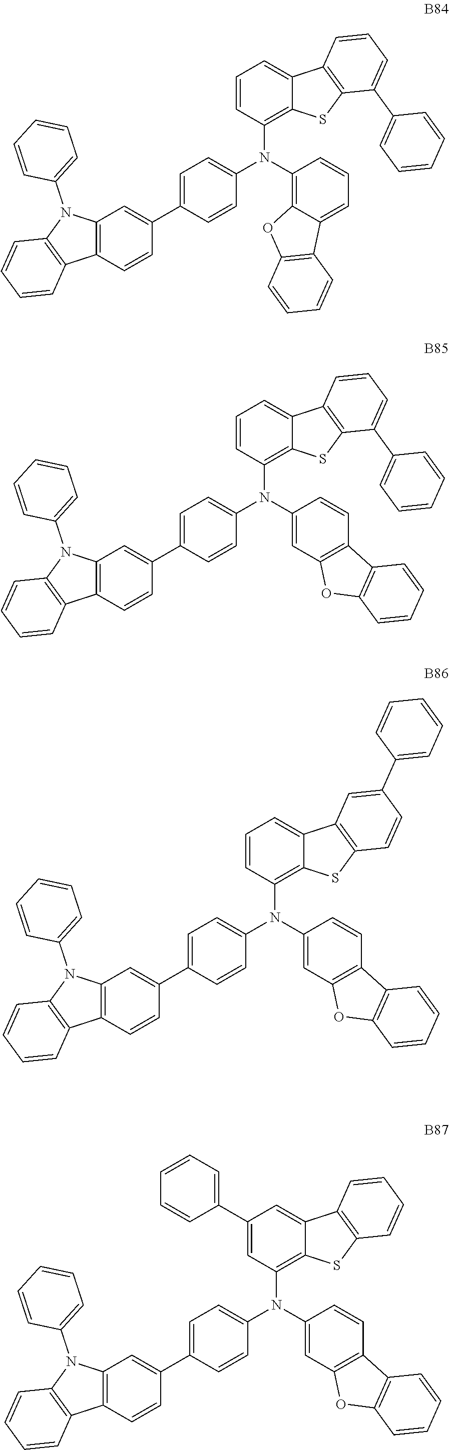 [SMILES: c1ccc(-c2cccc3c2sc2c(N(c4ccc(-c5ccc6c7ccccc7n(-c7ccccc7)c6c5)cc4)c4cccc5c4oc4ccccc45)cccc23)cc1 ; c1ccc(-c2cccc3c2sc2c(N(c4ccc(-c5ccc6c7ccccc7n(-c7ccccc7)c6c5)cc4)c4ccc5c(c4)oc4ccccc45)cccc23)cc1 ; c1ccc(-c2ccc3sc4c(N(c5ccc(-c6ccc7c8ccccc8n(-c8ccccc8)c7c6)cc5)c5ccc6c(c5)oc5ccccc56)cccc4c3c2)cc1 ; c1ccc(-c2cc(N(c3ccc(-c4ccc5c6ccccc6n(-c6ccccc6)c5c4)cc3)c3ccc4c(c3)oc3ccccc34)c3sc4ccccc4c3c2)cc1] 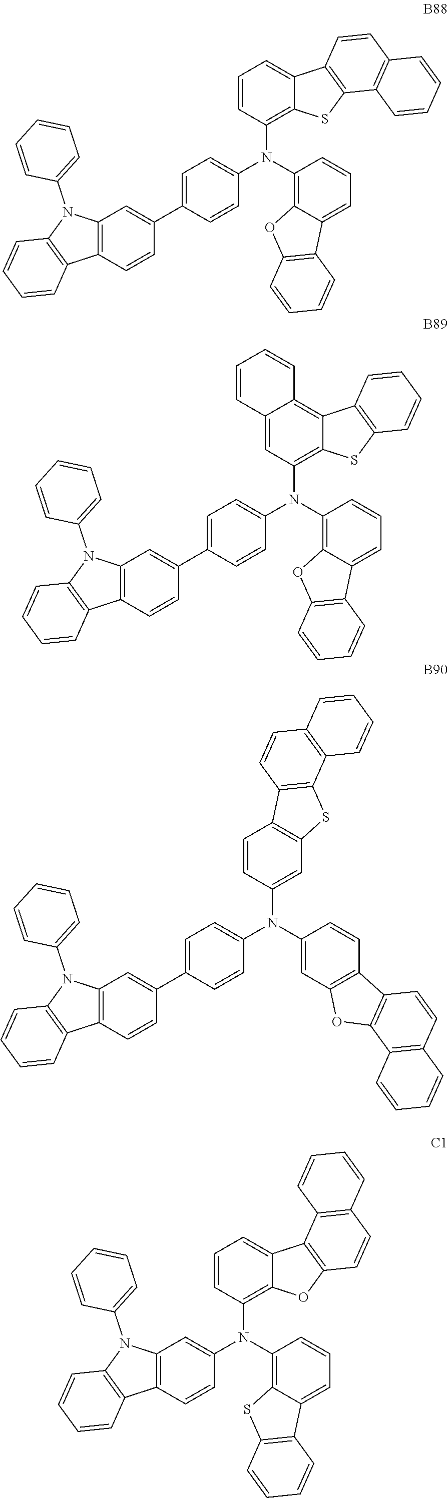 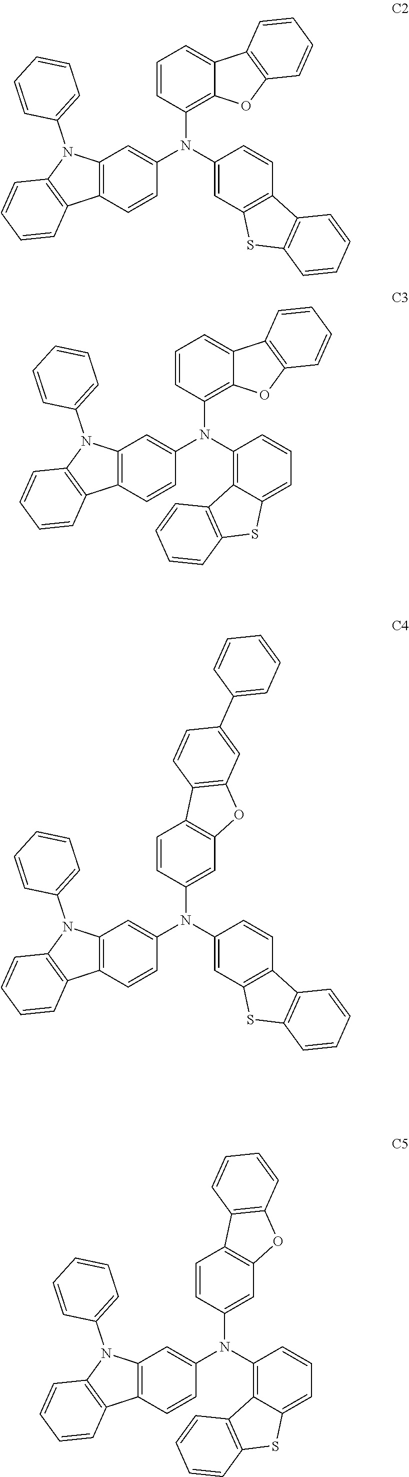 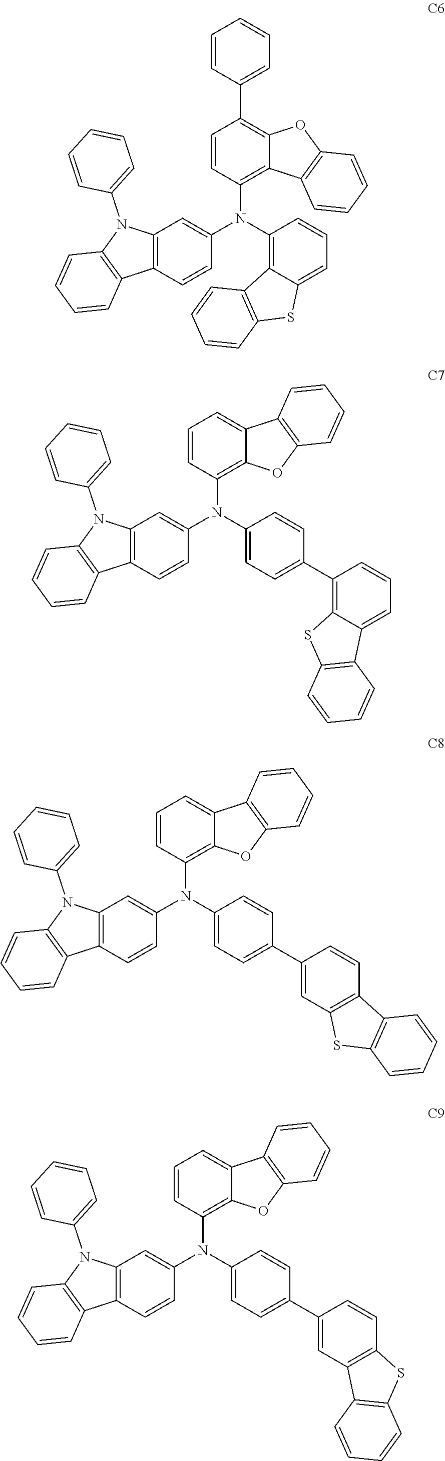 [SMILES: c1ccc(-c2ccc(N(c3ccc4c5ccccc5n(-c5ccccc5)c4c3)c3cccc4sc5ccccc5c34)c3c2oc2ccccc23)cc1 ; c1ccc(-n2c3ccccc3c3ccc(N(c4ccc(-c5cccc6c5sc5ccccc56)cc4)c4cccc5c4oc4ccccc45)cc32)cc1 ; c1ccc(-n2c3ccccc3c3ccc(N(c4ccc(-c5ccc6c(c5)sc5ccccc56)cc4)c4cccc5c4oc4ccccc45)cc32)cc1 ; c1ccc(-n2c3ccccc3c3ccc(N(c4ccc(-c5ccc6sc7ccccc7c6c5)cc4)c4cccc5c4oc4ccccc45)cc32)cc1] 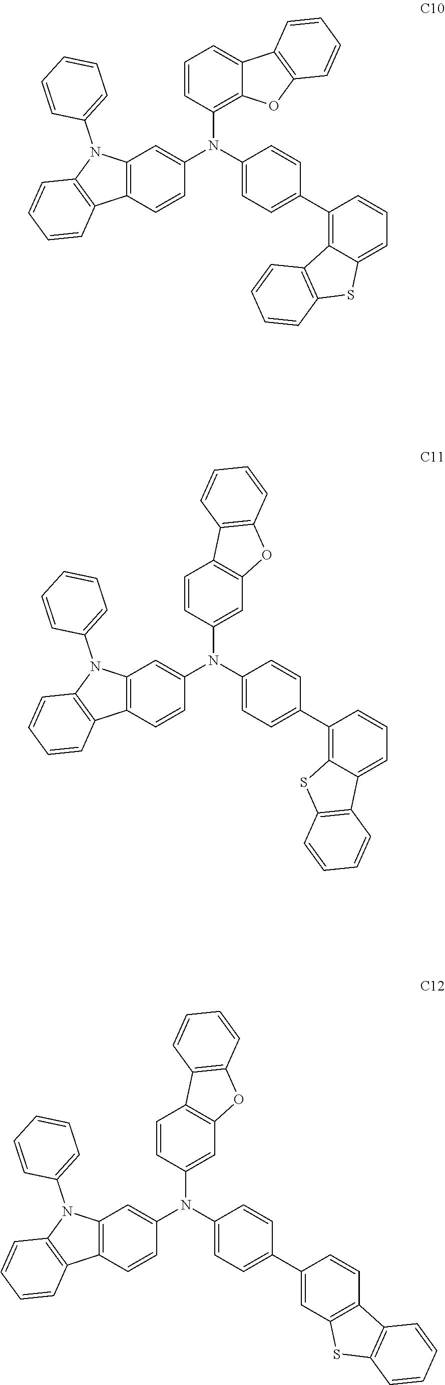 [SMILES: c1ccc(-n2c3ccccc3c3ccc(N(c4ccc(-c5cccc6sc7ccccc7c56)cc4)c4cccc5c4oc4ccccc45)cc32)cc1 ; c1ccc(-n2c3ccccc3c3ccc(N(c4ccc(-c5cccc6c5sc5ccccc56)cc4)c4ccc5c(c4)oc4ccccc45)cc32)cc1 ; c1ccc(-n2c3ccccc3c3ccc(N(c4ccc(-c5ccc6c(c5)sc5ccccc56)cc4)c4ccc5c(c4)oc4ccccc45)cc32)cc1] 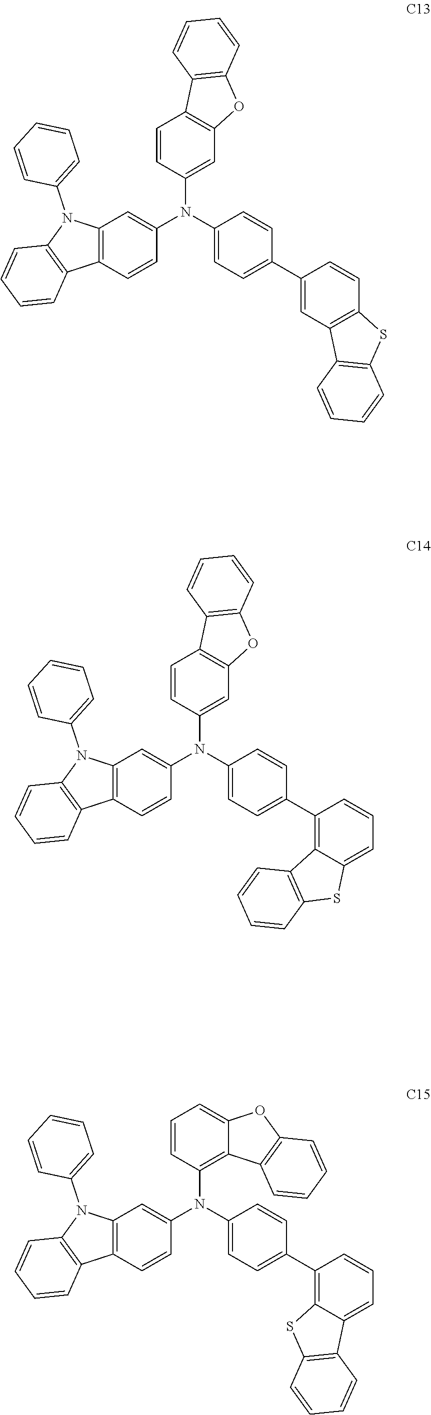 [SMILES: c1ccc(-n2c3ccccc3c3ccc(N(c4ccc(-c5ccc6sc7ccccc7c6c5)cc4)c4ccc5c(c4)oc4ccccc45)cc32)cc1 ; c1ccc(-n2c3ccccc3c3ccc(N(c4ccc(-c5cccc6sc7ccccc7c56)cc4)c4ccc5c(c4)oc4ccccc45)cc32)cc1 ; c1ccc(-n2c3ccccc3c3ccc(N(c4ccc(-c5cccc6c5sc5ccccc56)cc4)c4cccc5oc6ccccc6c45)cc32)cc1] 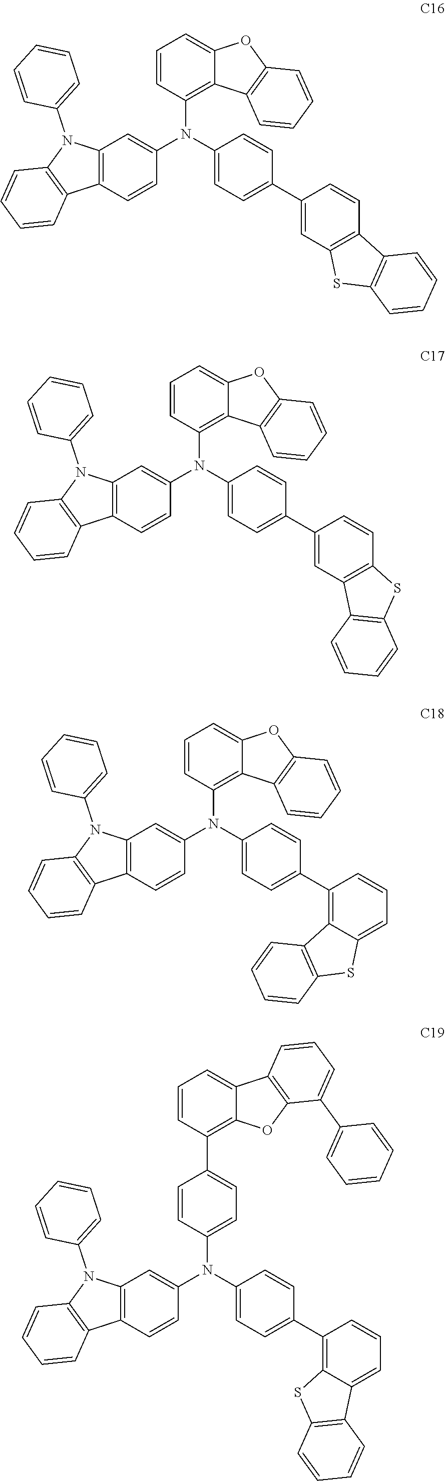 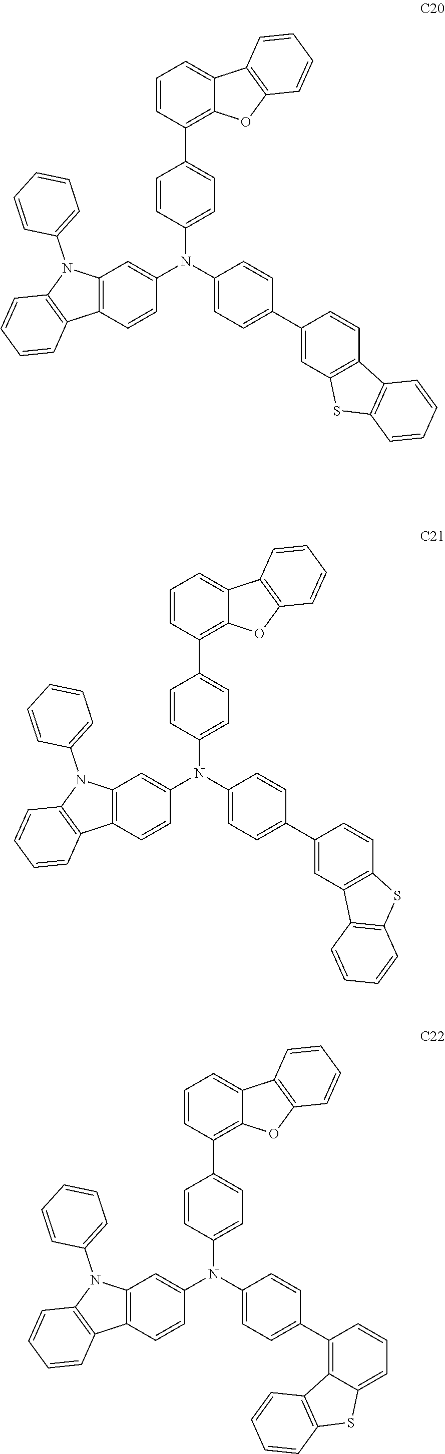 [SMILES: c1ccc(-n2c3ccccc3c3ccc(N(c4ccc(-c5ccc6c(c5)sc5ccccc56)cc4)c4ccc(-c5cccc6c5oc5ccccc56)cc4)cc32)cc1 ; c1ccc(-n2c3ccccc3c3ccc(N(c4ccc(-c5ccc6sc7ccccc7c6c5)cc4)c4ccc(-c5cccc6c5oc5ccccc56)cc4)cc32)cc1 ; c1ccc(-n2c3ccccc3c3ccc(N(c4ccc(-c5cccc6c5oc5ccccc56)cc4)c4ccc(-c5cccc6sc7ccccc7c56)cc4)cc32)cc1] 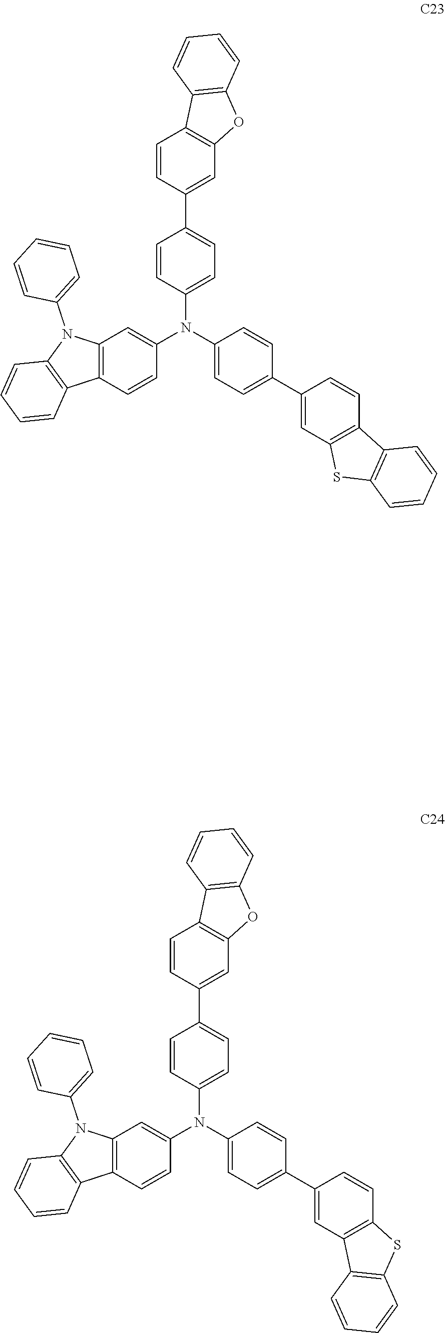 [SMILES: c1ccc(-n2c3ccccc3c3ccc(N(c4ccc(-c5ccc6c(c5)oc5ccccc56)cc4)c4ccc(-c5ccc6c(c5)sc5ccccc56)cc4)cc32)cc1 ; c1ccc(-n2c3ccccc3c3ccc(N(c4ccc(-c5ccc6c(c5)oc5ccccc56)cc4)c4ccc(-c5ccc6sc7ccccc7c6c5)cc4)cc32)cc1] 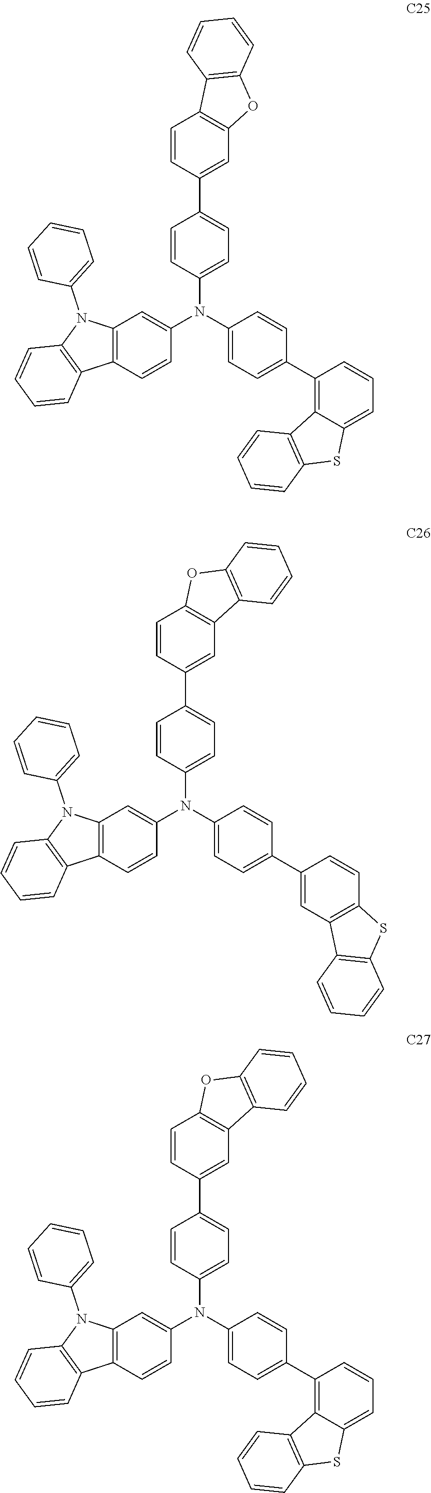 [SMILES: c1ccc(-n2c3ccccc3c3ccc(N(c4ccc(-c5ccc6c(c5)oc5ccccc56)cc4)c4ccc(-c5cccc6sc7ccccc7c56)cc4)cc32)cc1 ; c1ccc(-n2c3ccccc3c3ccc(N(c4ccc(-c5ccc6oc7ccccc7c6c5)cc4)c4ccc(-c5ccc6sc7ccccc7c6c5)cc4)cc32)cc1 ; c1ccc(-n2c3ccccc3c3ccc(N(c4ccc(-c5ccc6oc7ccccc7c6c5)cc4)c4ccc(-c5cccc6sc7ccccc7c56)cc4)cc32)cc1] 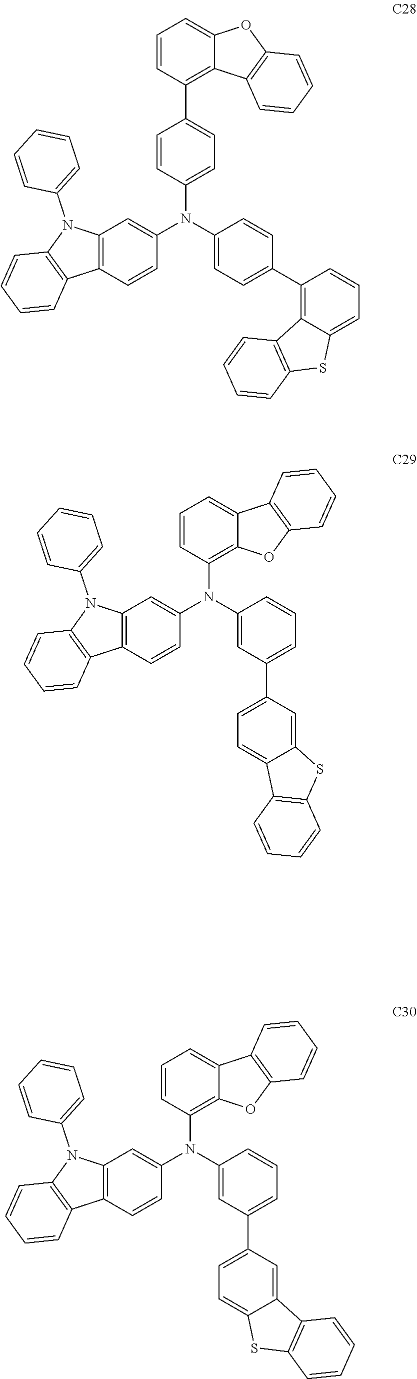 [SMILES: c1ccc(-n2c3ccccc3c3ccc(N(c4ccc(-c5cccc6oc7ccccc7c56)cc4)c4ccc(-c5cccc6sc7ccccc7c56)cc4)cc32)cc1 ; c1ccc(-n2c3ccccc3c3ccc(N(c4cccc(-c5ccc6c(c5)sc5ccccc56)c4)c4cccc5c4oc4ccccc45)cc32)cc1 ; c1ccc(-n2c3ccccc3c3ccc(N(c4cccc(-c5ccc6sc7ccccc7c6c5)c4)c4cccc5c4oc4ccccc45)cc32)cc1] 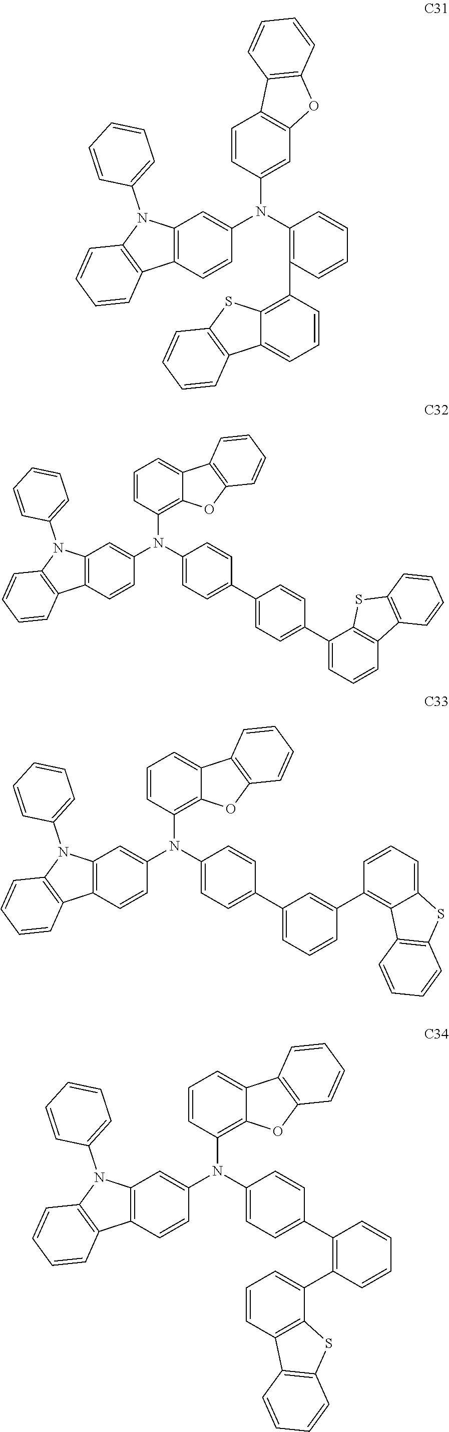 [SMILES: c1ccc(-n2c3ccccc3c3ccc(N(c4ccc5c(c4)oc4ccccc45)c4ccccc4-c4cccc5c4sc4ccccc45)cc32)cc1 ; c1ccc(-n2c3ccccc3c3ccc(N(c4ccc(-c5ccc(-c6cccc7c6sc6ccccc67)cc5)cc4)c4cccc5c4oc4ccccc45)cc32)cc1 ; c1ccc(-n2c3ccccc3c3ccc(N(c4ccc(-c5cccc(-c6cccc7sc8ccccc8c67)c5)cc4)c4cccc5c4oc4ccccc45)cc32)cc1 ; c1ccc(-n2c3ccccc3c3ccc(N(c4ccc(-c5ccccc5-c5cccc6c5sc5ccccc56)cc4)c4cccc5c4oc4ccccc45)cc32)cc1] 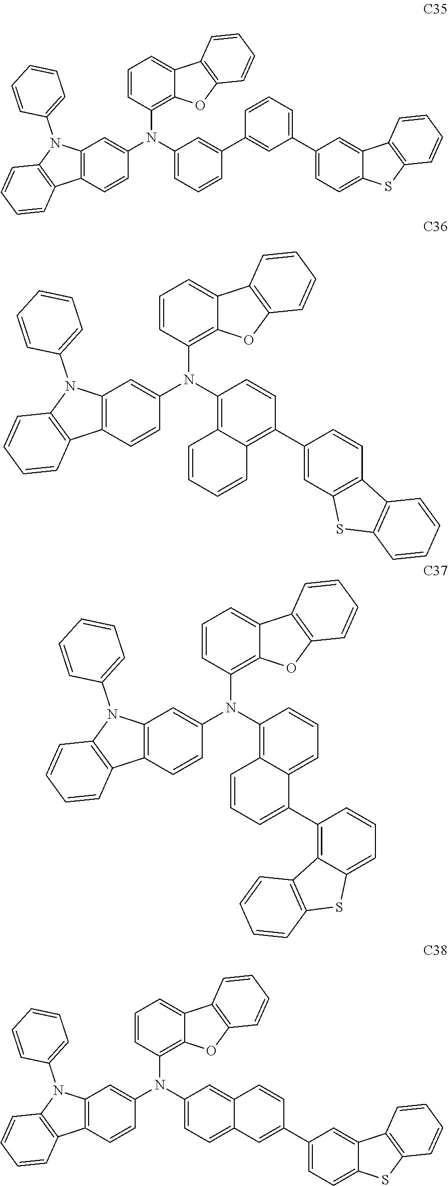 [SMILES: c1ccc(-n2c3ccccc3c3ccc(N(c4cccc(-c5cccc(-c6ccc7sc8ccccc8c7c6)c5)c4)c4cccc5c4oc4ccccc45)cc32)cc1 ; c1ccc(-n2c3ccccc3c3ccc(N(c4ccc(-c5ccc6c(c5)sc5ccccc56)c5ccccc45)c4cccc5c4oc4ccccc45)cc32)cc1 ; c1ccc(-n2c3ccccc3c3ccc(N(c4cccc5c(-c6cccc7sc8ccccc8c67)cccc45)c4cccc5c4oc4ccccc45)cc32)cc1 ; c1ccc(-n2c3ccccc3c3ccc(N(c4ccc5cc(-c6ccc7sc8ccccc8c7c6)ccc5c4)c4cccc5c4oc4ccccc45)cc32)cc1] 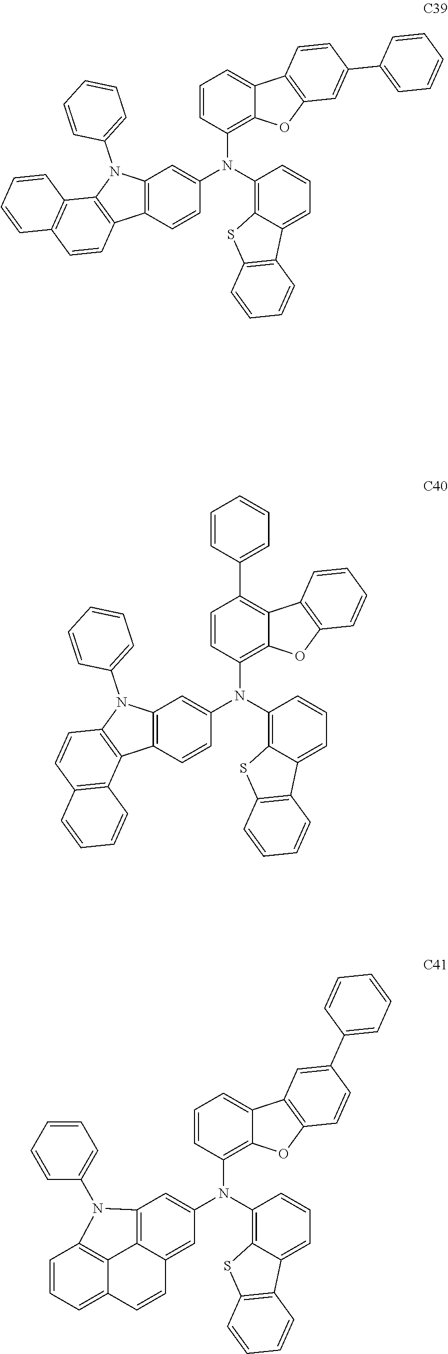 [SMILES: c1ccc(-c2ccc3c(c2)oc2c(N(c4ccc5c6ccc7ccccc7c6n(-c6ccccc6)c5c4)c4cccc5c4sc4ccccc45)cccc23)cc1 ; c1ccc(-c2ccc(N(c3ccc4c5c6ccccc6ccc5n(-c5ccccc5)c4c3)c3cccc4c3sc3ccccc34)c3oc4ccccc4c23)cc1 ; c1ccc(-c2ccc3oc4c(N(c5cc6ccc7cccc8c7c6c(c5)n8-c5ccccc5)c5cccc6c5sc5ccccc56)cccc4c3c2)cc1] 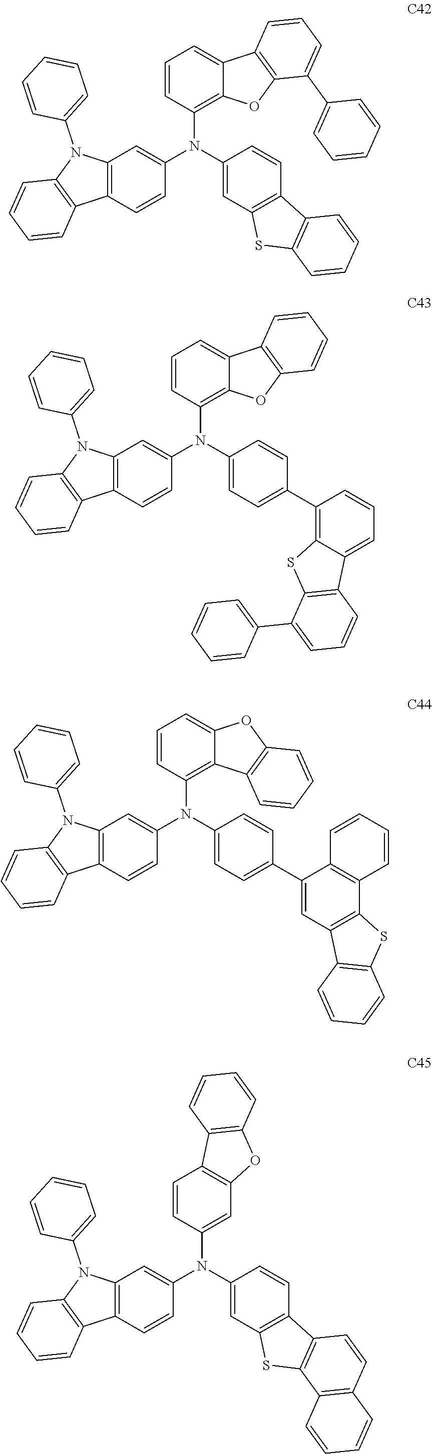 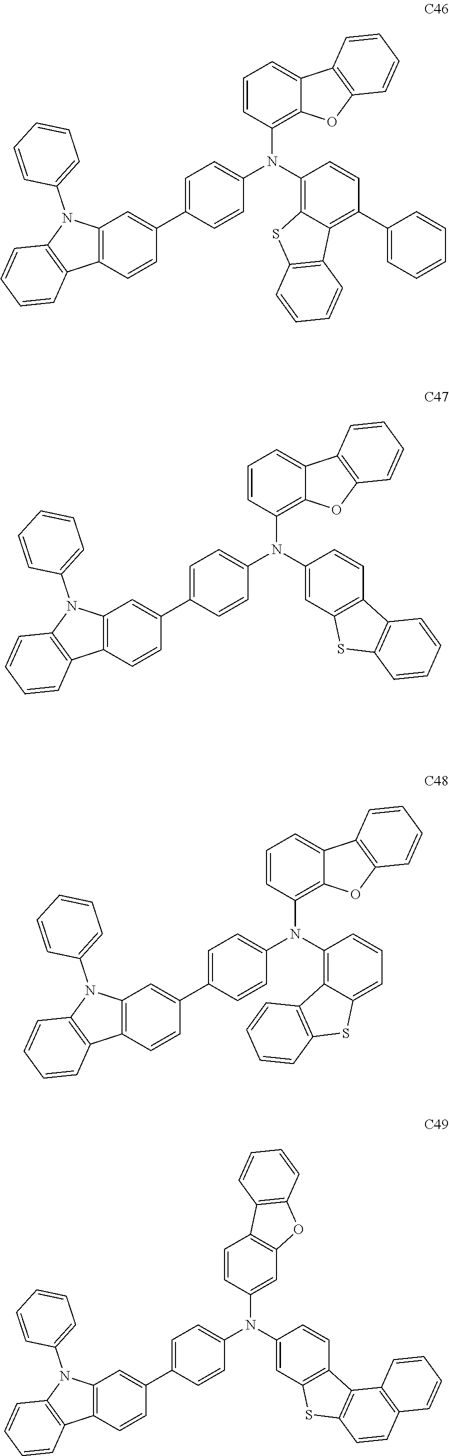 [SMILES: c1ccc(-c2ccc(N(c3ccc(-c4ccc5c6ccccc6n(-c6ccccc6)c5c4)cc3)c3cccc4c3oc3ccccc34)c3sc4ccccc4c23)cc1 ; c1ccc(-n2c3ccccc3c3ccc(-c4ccc(N(c5ccc6c(c5)sc5ccccc56)c5cccc6c5oc5ccccc56)cc4)cc32)cc1 ; c1ccc(-n2c3ccccc3c3ccc(-c4ccc(N(c5cccc6c5oc5ccccc56)c5cccc6sc7ccccc7c56)cc4)cc32)cc1 ; c1ccc(-n2c3ccccc3c3ccc(-c4ccc(N(c5ccc6c(c5)oc5ccccc56)c5ccc6c(c5)sc5ccc7ccccc7c56)cc4)cc32)cc1] 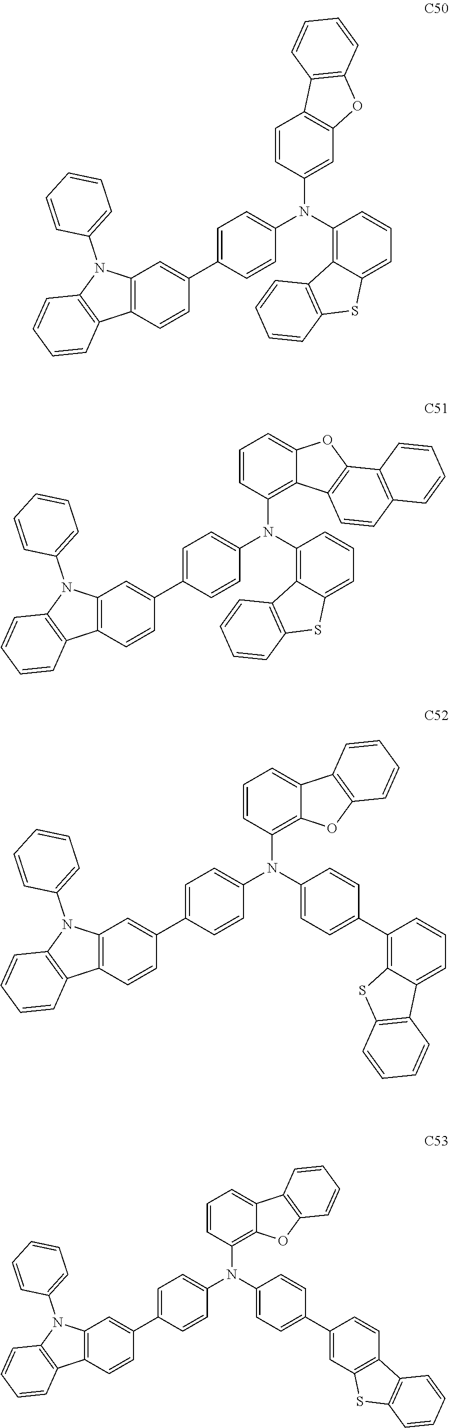 [SMILES: c1ccc(-n2c3ccccc3c3ccc(-c4ccc(N(c5ccc6c(c5)oc5ccccc56)c5cccc6sc7ccccc7c56)cc4)cc32)cc1 ; c1ccc(-n2c3ccccc3c3ccc(-c4ccc(N(c5cccc6oc7c8ccccc8ccc7c56)c5cccc6sc7ccccc7c56)cc4)cc32)cc1 ; c1ccc(-n2c3ccccc3c3ccc(-c4ccc(N(c5ccc(-c6cccc7c6sc6ccccc67)cc5)c5cccc6c5oc5ccccc56)cc4)cc32)cc1 ; c1ccc(-n2c3ccccc3c3ccc(-c4ccc(N(c5ccc(-c6ccc7c(c6)sc6ccccc67)cc5)c5cccc6c5oc5ccccc56)cc4)cc32)cc1] 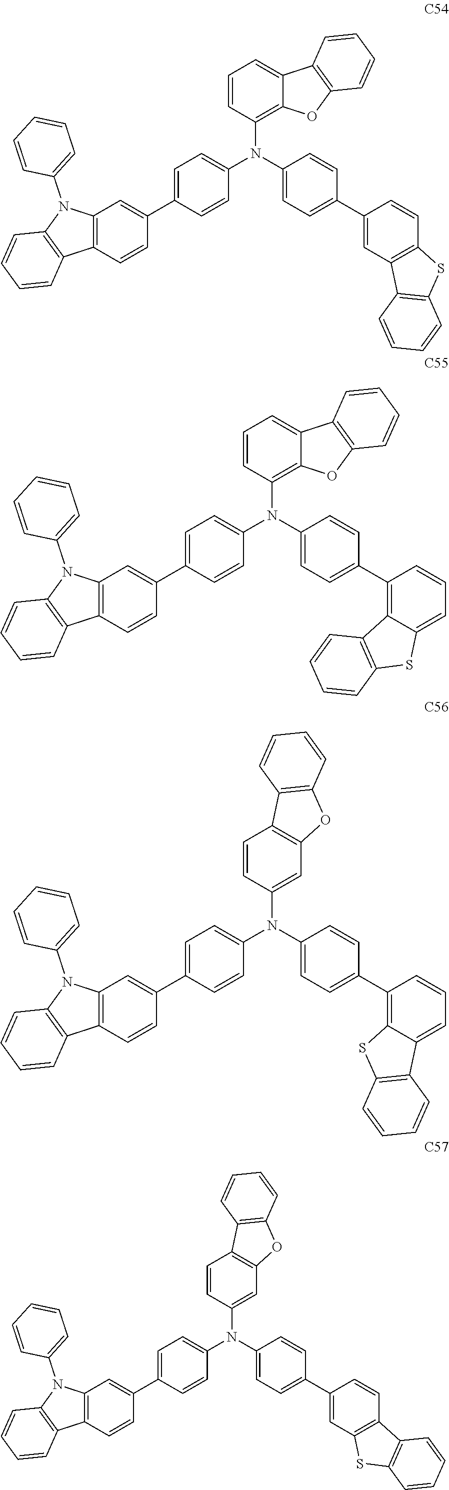 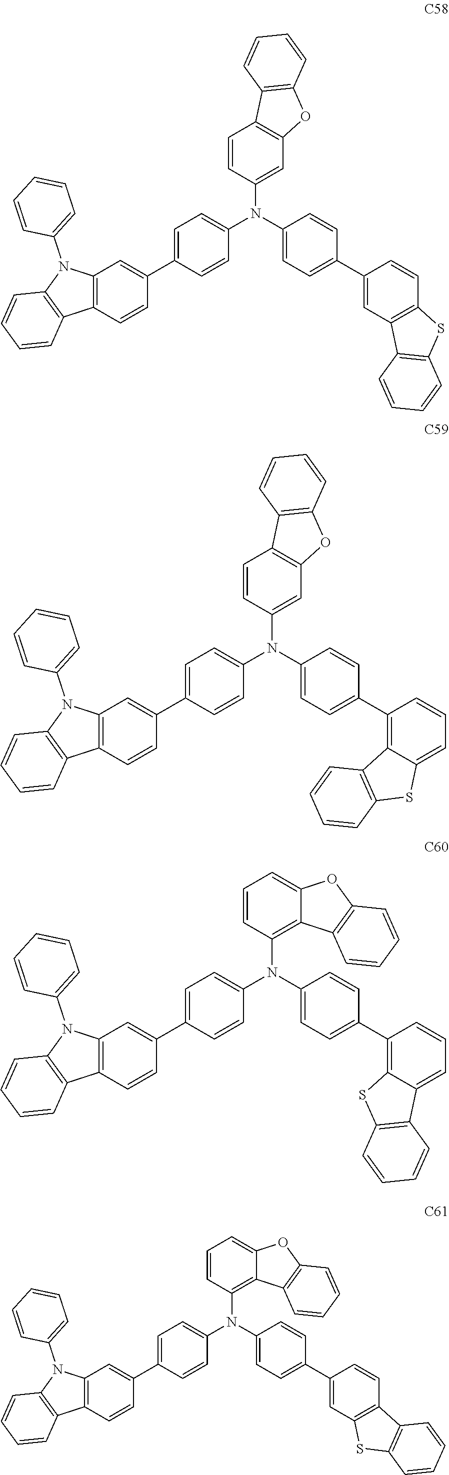 [SMILES: c1ccc(-n2c3ccccc3c3ccc(-c4ccc(N(c5ccc(-c6ccc7sc8ccccc8c7c6)cc5)c5ccc6c(c5)oc5ccccc56)cc4)cc32)cc1 ; c1ccc(-n2c3ccccc3c3ccc(-c4ccc(N(c5ccc(-c6cccc7sc8ccccc8c67)cc5)c5ccc6c(c5)oc5ccccc56)cc4)cc32)cc1 ; c1ccc(-n2c3ccccc3c3ccc(-c4ccc(N(c5ccc(-c6cccc7c6sc6ccccc67)cc5)c5cccc6oc7ccccc7c56)cc4)cc32)cc1 ; c1ccc(-n2c3ccccc3c3ccc(-c4ccc(N(c5ccc(-c6ccc7c(c6)sc6ccccc67)cc5)c5cccc6oc7ccccc7c56)cc4)cc32)cc1] 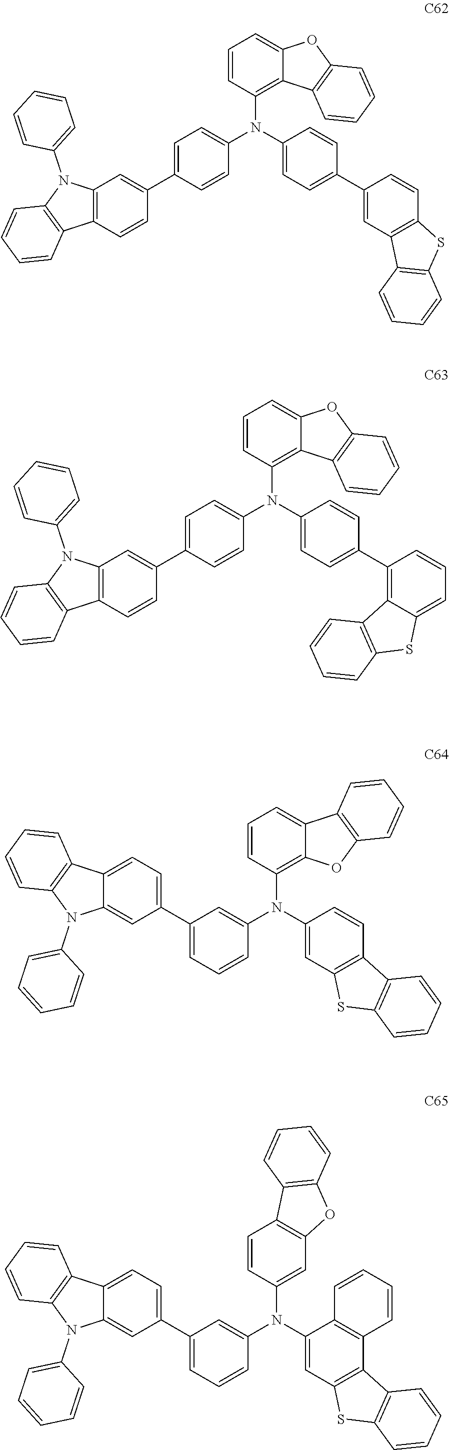 [SMILES: c1ccc(-n2c3ccccc3c3ccc(-c4ccc(N(c5ccc(-c6ccc7sc8ccccc8c7c6)cc5)c5cccc6oc7ccccc7c56)cc4)cc32)cc1 ; c1ccc(-n2c3ccccc3c3ccc(-c4ccc(N(c5ccc(-c6cccc7sc8ccccc8c67)cc5)c5cccc6oc7ccccc7c56)cc4)cc32)cc1 ; c1ccc(-n2c3ccccc3c3ccc(-c4cccc(N(c5ccc6c(c5)sc5ccccc56)c5cccc6c5oc5ccccc56)c4)cc32)cc1 ; c1ccc(-n2c3ccccc3c3ccc(-c4cccc(N(c5ccc6c(c5)oc5ccccc56)c5cc6sc7ccccc7c6c6ccccc56)c4)cc32)cc1] 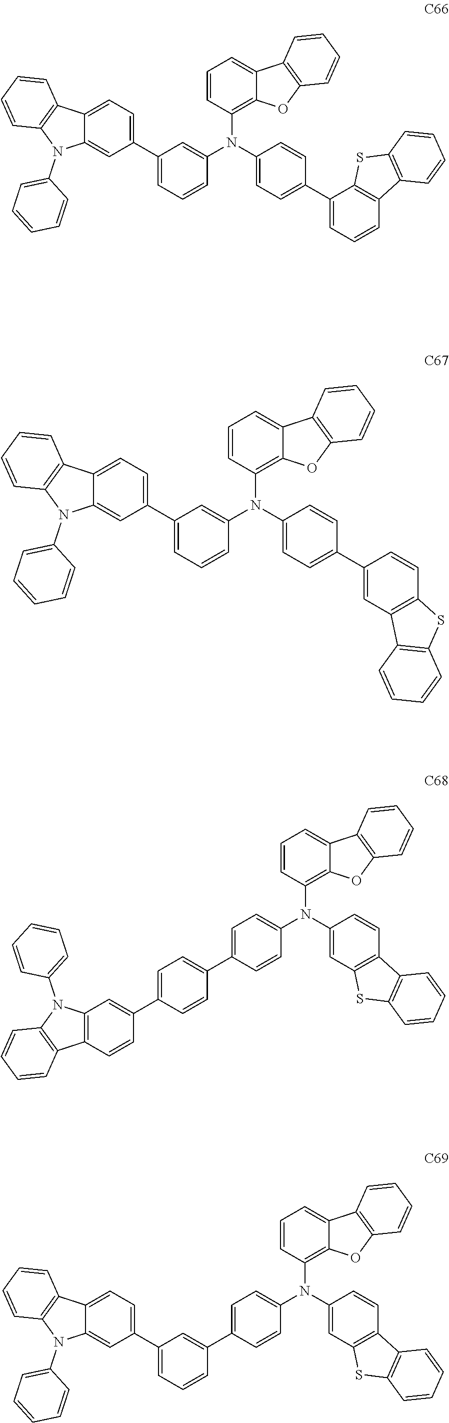 [SMILES: c1ccc(-n2c3ccccc3c3ccc(-c4cccc(N(c5ccc(-c6cccc7c6sc6ccccc67)cc5)c5cccc6c5oc5ccccc56)c4)cc32)cc1 ; c1ccc(-n2c3ccccc3c3ccc(-c4cccc(N(c5ccc(-c6ccc7sc8ccccc8c7c6)cc5)c5cccc6c5oc5ccccc56)c4)cc32)cc1 ; c1ccc(-n2c3ccccc3c3ccc(-c4ccc(-c5ccc(N(c6ccc7c(c6)sc6ccccc67)c6cccc7c6oc6ccccc67)cc5)cc4)cc32)cc1 ; c1ccc(-n2c3ccccc3c3ccc(-c4cccc(-c5ccc(N(c6ccc7c(c6)sc6ccccc67)c6cccc7c6oc6ccccc67)cc5)c4)cc32)cc1] 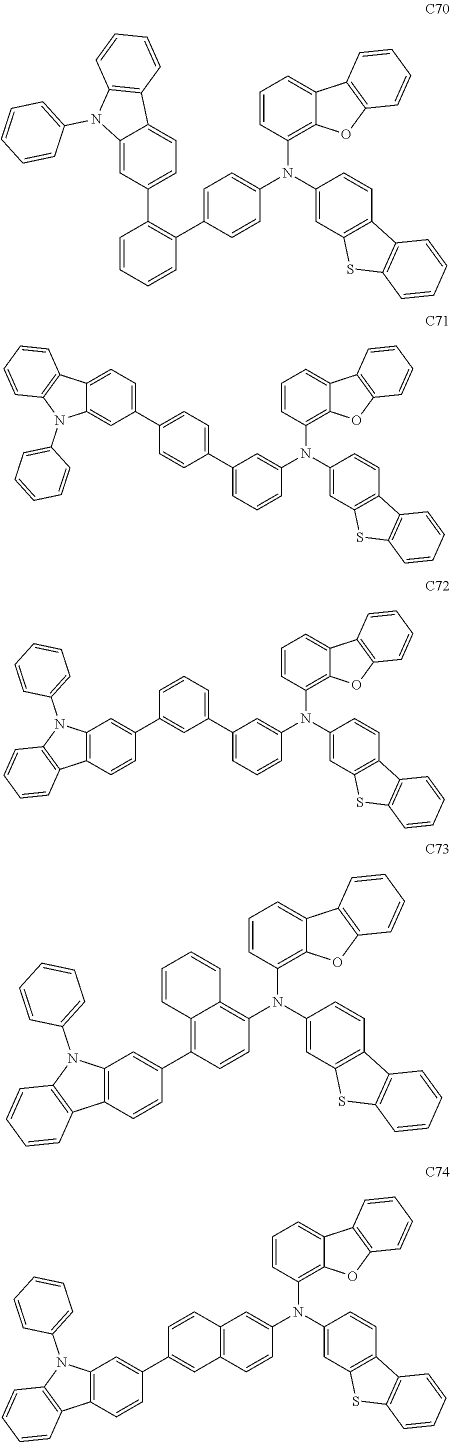 [SMILES: c1ccc(-n2c3ccccc3c3ccc(-c4ccccc4-c4ccc(N(c5ccc6c(c5)sc5ccccc56)c5cccc6c5oc5ccccc56)cc4)cc32)cc1 ; c1ccc(-n2c3ccccc3c3ccc(-c4ccc(-c5cccc(N(c6ccc7c(c6)sc6ccccc67)c6cccc7c6oc6ccccc67)c5)cc4)cc32)cc1 ; c1ccc(-n2c3ccccc3c3ccc(-c4cccc(-c5cccc(N(c6ccc7c(c6)sc6ccccc67)c6cccc7c6oc6ccccc67)c5)c4)cc32)cc1 ; c1ccc(-n2c3ccccc3c3ccc(-c4ccc(N(c5ccc6c(c5)sc5ccccc56)c5cccc6c5oc5ccccc56)c5ccccc45)cc32)cc1 ; c1ccc(-n2c3ccccc3c3ccc(-c4ccc5cc(N(c6ccc7c(c6)sc6ccccc67)c6cccc7c6oc6ccccc67)ccc5c4)cc32)cc1] 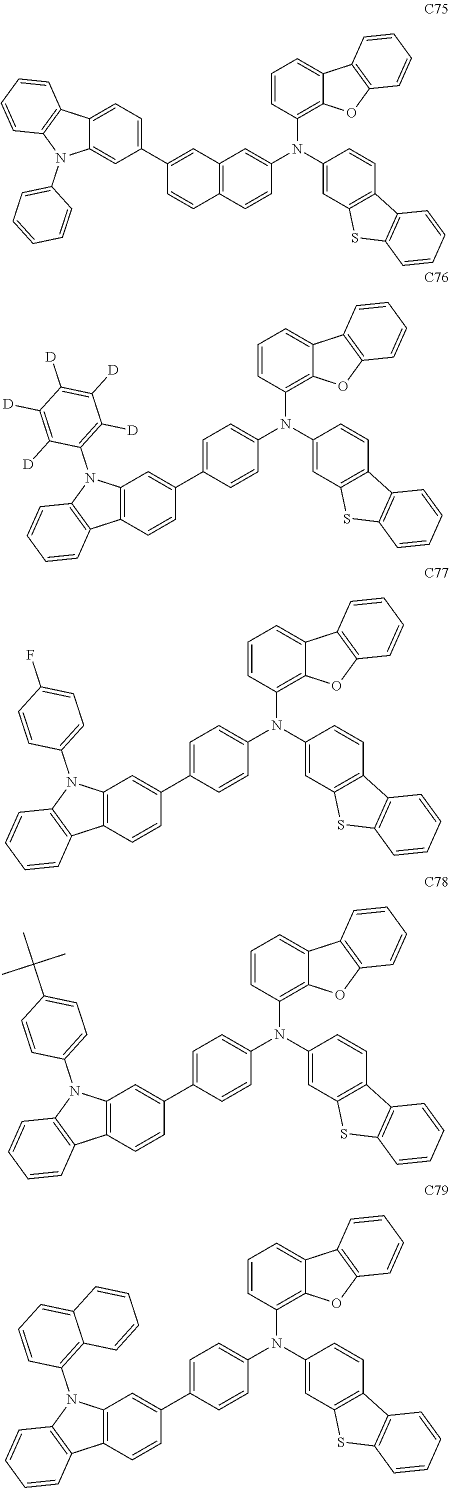 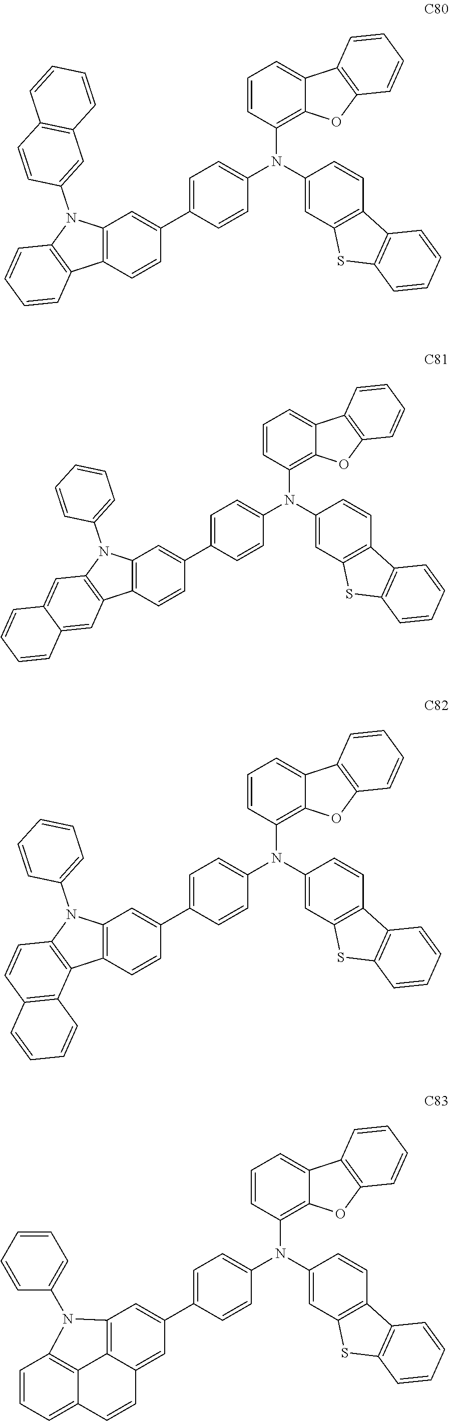 [SMILES: c1ccc2cc(-n3c4ccccc4c4ccc(-c5ccc(N(c6ccc7c(c6)sc6ccccc67)c6cccc7c6oc6ccccc67)cc5)cc43)ccc2c1 ; c1ccc(-n2c3cc(-c4ccc(N(c5ccc6c(c5)sc5ccccc56)c5cccc6c5oc5ccccc56)cc4)ccc3c3cc4ccccc4cc32)cc1 ; c1ccc(-n2c3cc(-c4ccc(N(c5ccc6c(c5)sc5ccccc56)c5cccc6c5oc5ccccc56)cc4)ccc3c3c4ccccc4ccc32)cc1 ; c1ccc(-n2c3cccc4ccc5cc(-c6ccc(N(c7ccc8c(c7)sc7ccccc78)c7cccc8c7oc7ccccc78)cc6)cc2c5c43)cc1] 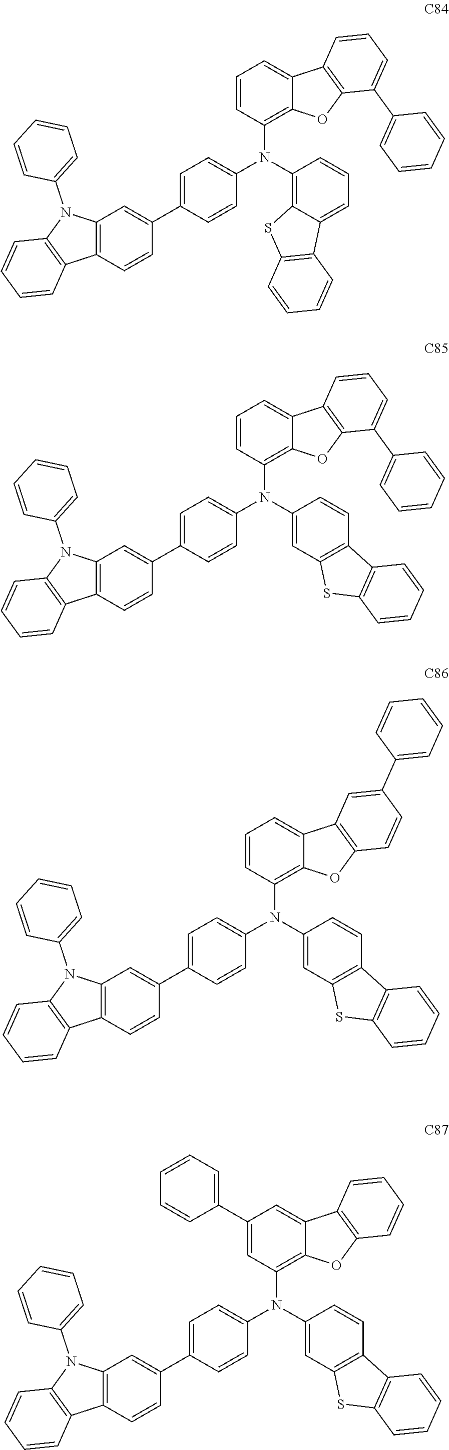 [SMILES: c1ccc(-c2cccc3c2oc2c(N(c4ccc(-c5ccc6c7ccccc7n(-c7ccccc7)c6c5)cc4)c4cccc5c4sc4ccccc45)cccc23)cc1 ; c1ccc(-c2cccc3c2oc2c(N(c4ccc(-c5ccc6c7ccccc7n(-c7ccccc7)c6c5)cc4)c4ccc5c(c4)sc4ccccc45)cccc23)cc1 ; c1ccc(-c2ccc3oc4c(N(c5ccc(-c6ccc7c8ccccc8n(-c8ccccc8)c7c6)cc5)c5ccc6c(c5)sc5ccccc56)cccc4c3c2)cc1 ; c1ccc(-c2cc(N(c3ccc(-c4ccc5c6ccccc6n(-c6ccccc6)c5c4)cc3)c3ccc4c(c3)sc3ccccc34)c3oc4ccccc4c3c2)cc1] 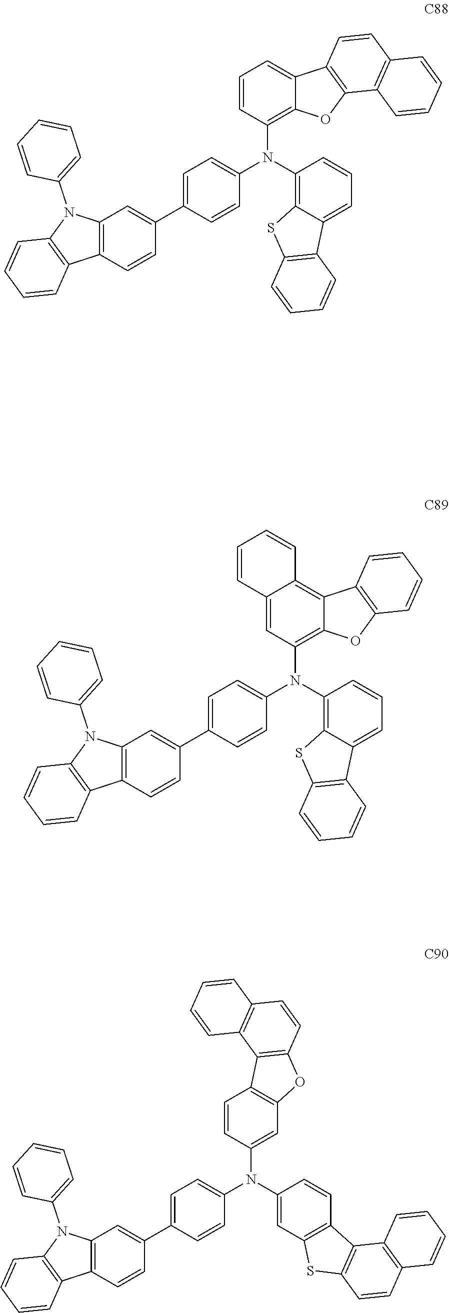 [SMILES: c1ccc(-n2c3ccccc3c3ccc(-c4ccc(N(c5cccc6c5oc5c7ccccc7ccc65)c5cccc6c5sc5ccccc56)cc4)cc32)cc1 ; c1ccc(-n2c3ccccc3c3ccc(-c4ccc(N(c5cc6ccccc6c6c5oc5ccccc56)c5cccc6c5sc5ccccc56)cc4)cc32)cc1 ; c1ccc(-n2c3ccccc3c3ccc(-c4ccc(N(c5ccc6c(c5)oc5ccc7ccccc7c56)c5ccc6c(c5)sc5ccc7ccccc7c56)cc4)cc32)cc1] 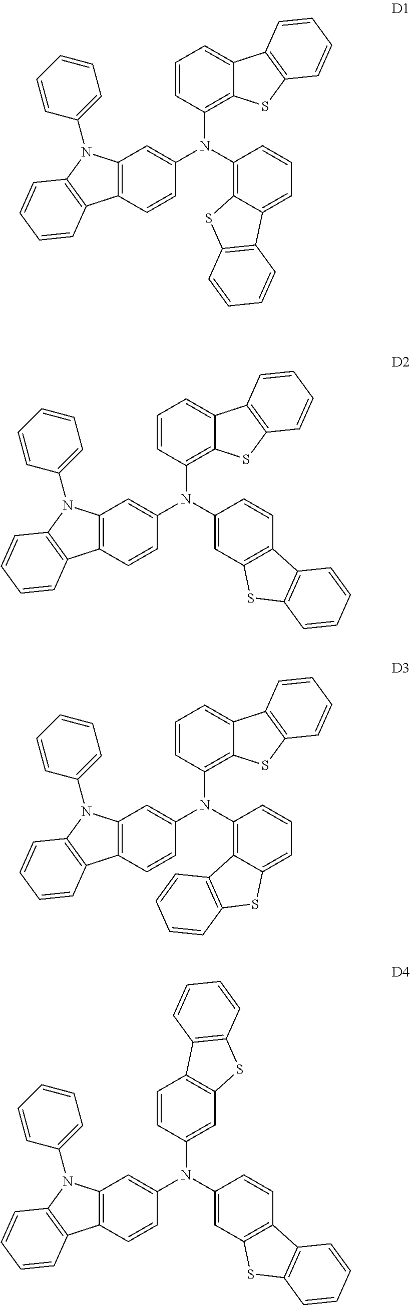 [SMILES: c1ccc(-n2c3ccccc3c3ccc(N(c4cccc5c4sc4ccccc45)c4cccc5c4sc4ccccc45)cc32)cc1 ; c1ccc(-n2c3ccccc3c3ccc(N(c4ccc5c(c4)sc4ccccc45)c4cccc5c4sc4ccccc45)cc32)cc1 ; c1ccc(-n2c3ccccc3c3ccc(N(c4cccc5c4sc4ccccc45)c4cccc5sc6ccccc6c45)cc32)cc1 ; c1ccc(-n2c3ccccc3c3ccc(N(c4ccc5c(c4)sc4ccccc45)c4ccc5c(c4)sc4ccccc45)cc32)cc1] 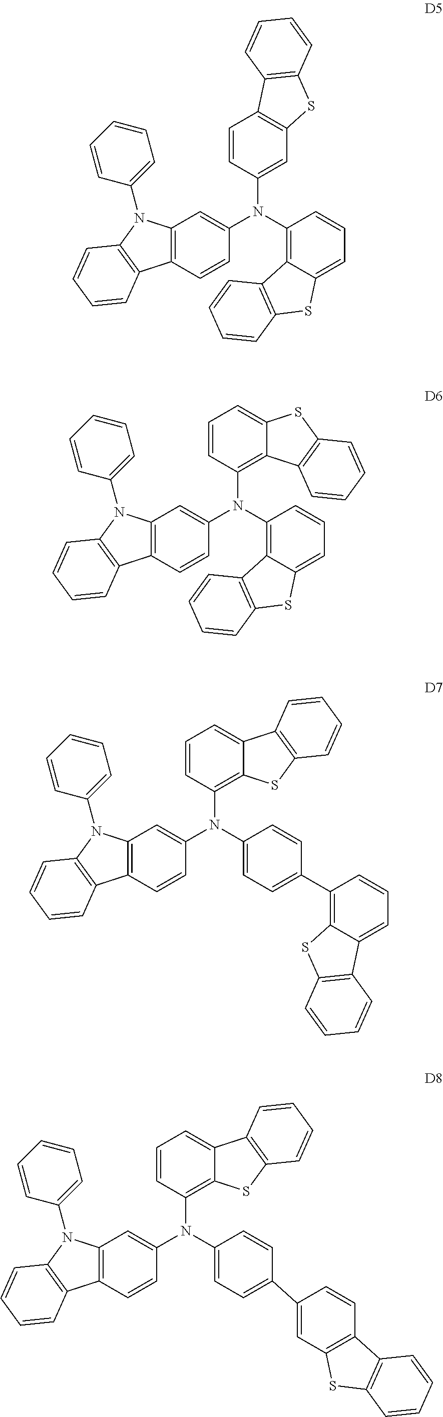 [SMILES: c1ccc(-n2c3ccccc3c3ccc(N(c4ccc5c(c4)sc4ccccc45)c4cccc5sc6ccccc6c45)cc32)cc1 ; c1ccc(-n2c3ccccc3c3ccc(N(c4cccc5sc6ccccc6c45)c4cccc5sc6ccccc6c45)cc32)cc1 ; c1ccc(-n2c3ccccc3c3ccc(N(c4ccc(-c5cccc6c5sc5ccccc56)cc4)c4cccc5c4sc4ccccc45)cc32)cc1 ; c1ccc(-n2c3ccccc3c3ccc(N(c4ccc(-c5ccc6c(c5)sc5ccccc56)cc4)c4cccc5c4sc4ccccc45)cc32)cc1] 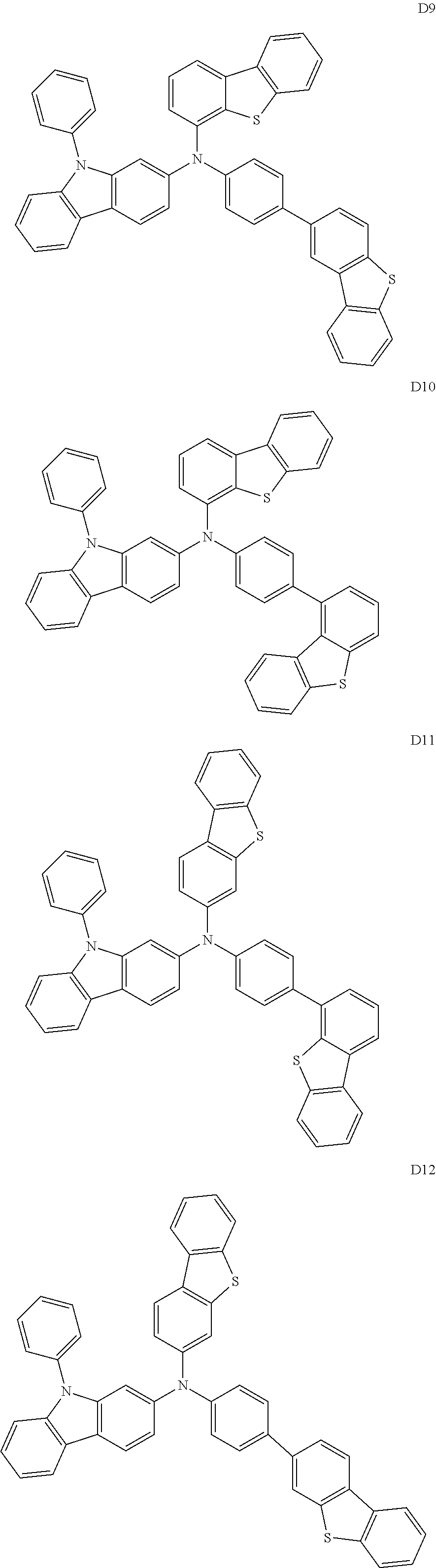 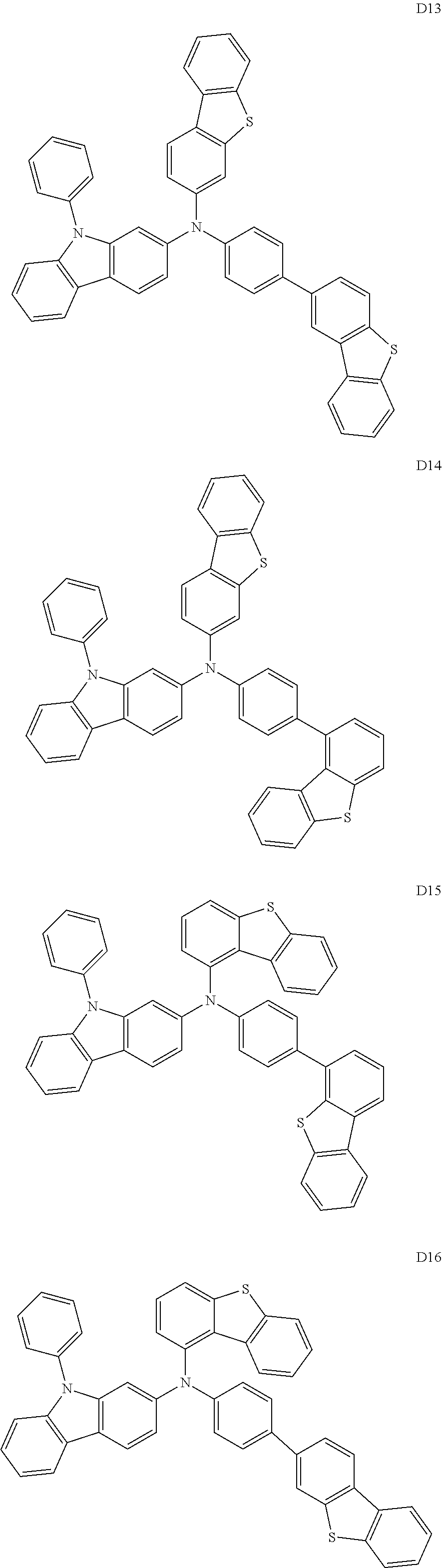 [SMILES: c1ccc(-n2c3ccccc3c3ccc(N(c4ccc(-c5ccc6sc7ccccc7c6c5)cc4)c4ccc5c(c4)sc4ccccc45)cc32)cc1 ; c1ccc(-n2c3ccccc3c3ccc(N(c4ccc(-c5cccc6sc7ccccc7c56)cc4)c4ccc5c(c4)sc4ccccc45)cc32)cc1 ; c1ccc(-n2c3ccccc3c3ccc(N(c4ccc(-c5cccc6c5sc5ccccc56)cc4)c4cccc5sc6ccccc6c45)cc32)cc1 ; c1ccc(-n2c3ccccc3c3ccc(N(c4ccc(-c5ccc6c(c5)sc5ccccc56)cc4)c4cccc5sc6ccccc6c45)cc32)cc1] 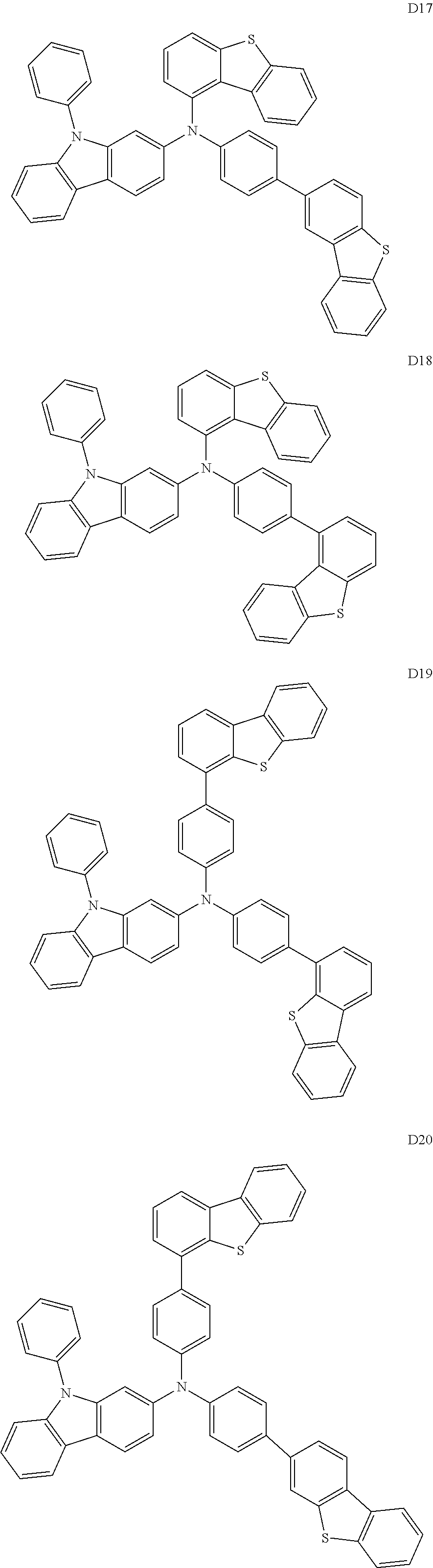 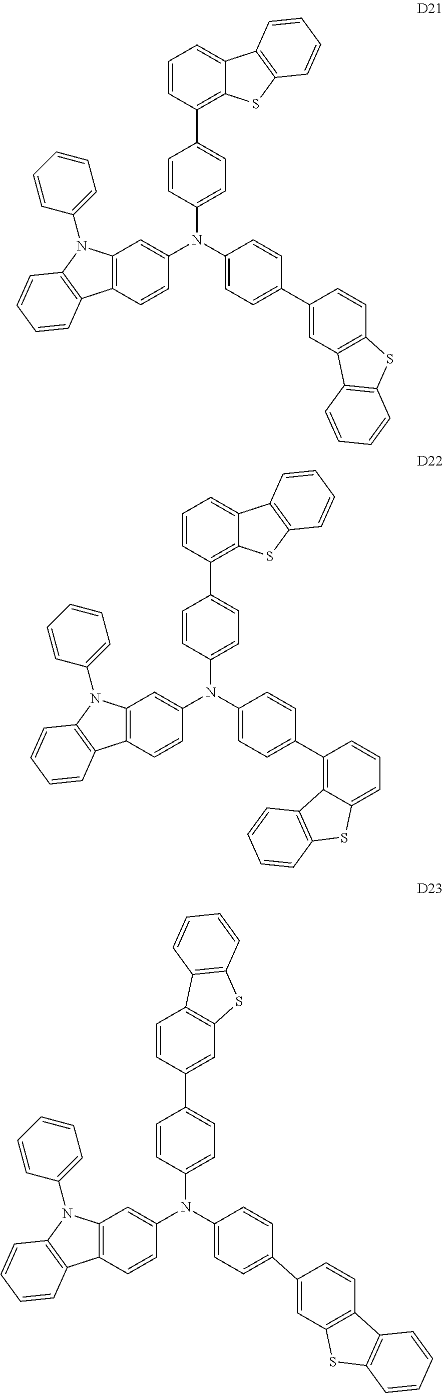 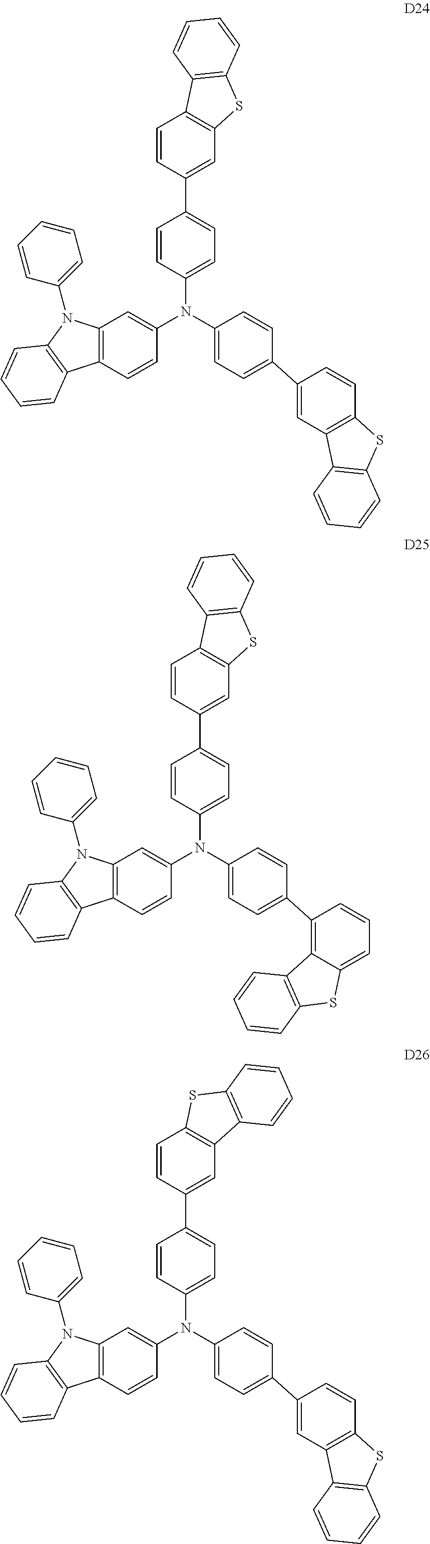 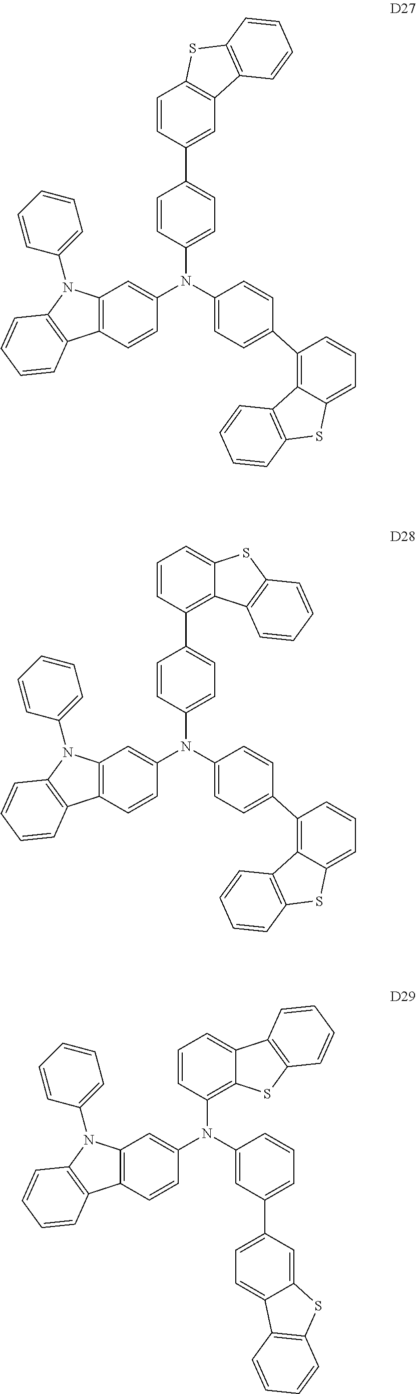 [SMILES: c1ccc(-n2c3ccccc3c3ccc(N(c4ccc(-c5ccc6sc7ccccc7c6c5)cc4)c4ccc(-c5cccc6sc7ccccc7c56)cc4)cc32)cc1 ; c1ccc(-n2c3ccccc3c3ccc(N(c4ccc(-c5cccc6sc7ccccc7c56)cc4)c4ccc(-c5cccc6sc7ccccc7c56)cc4)cc32)cc1 ; c1ccc(-n2c3ccccc3c3ccc(N(c4cccc(-c5ccc6c(c5)sc5ccccc56)c4)c4cccc5c4sc4ccccc45)cc32)cc1] 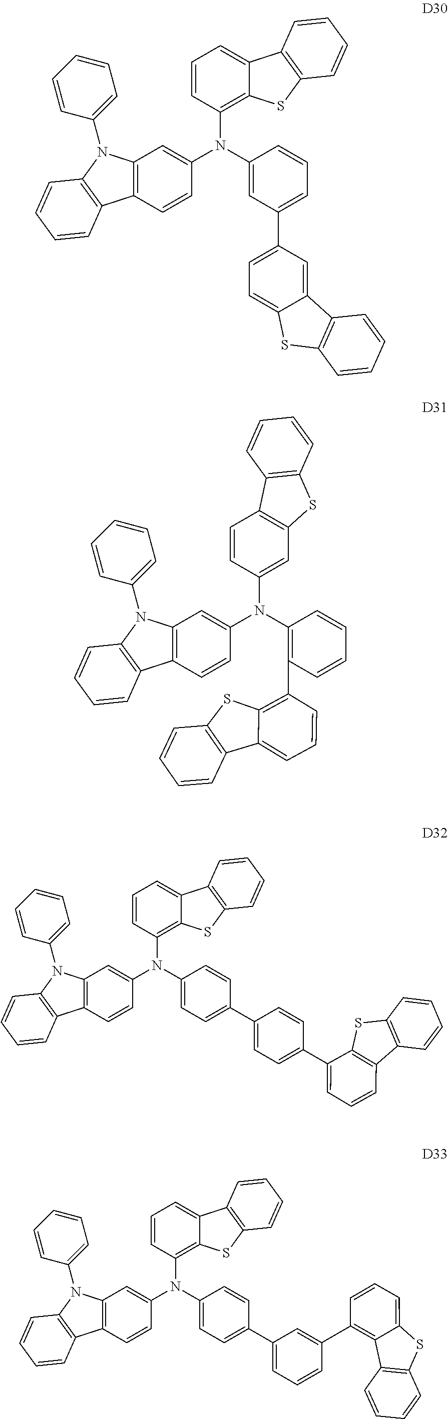 [SMILES: c1ccc(-n2c3ccccc3c3ccc(N(c4cccc(-c5ccc6sc7ccccc7c6c5)c4)c4cccc5c4sc4ccccc45)cc32)cc1 ; c1ccc(-n2c3ccccc3c3ccc(N(c4ccc5c(c4)sc4ccccc45)c4ccccc4-c4cccc5c4sc4ccccc45)cc32)cc1 ; c1ccc(-n2c3ccccc3c3ccc(N(c4ccc(-c5ccc(-c6cccc7c6sc6ccccc67)cc5)cc4)c4cccc5c4sc4ccccc45)cc32)cc1 ; c1ccc(-n2c3ccccc3c3ccc(N(c4ccc(-c5cccc(-c6cccc7sc8ccccc8c67)c5)cc4)c4cccc5c4sc4ccccc45)cc32)cc1] 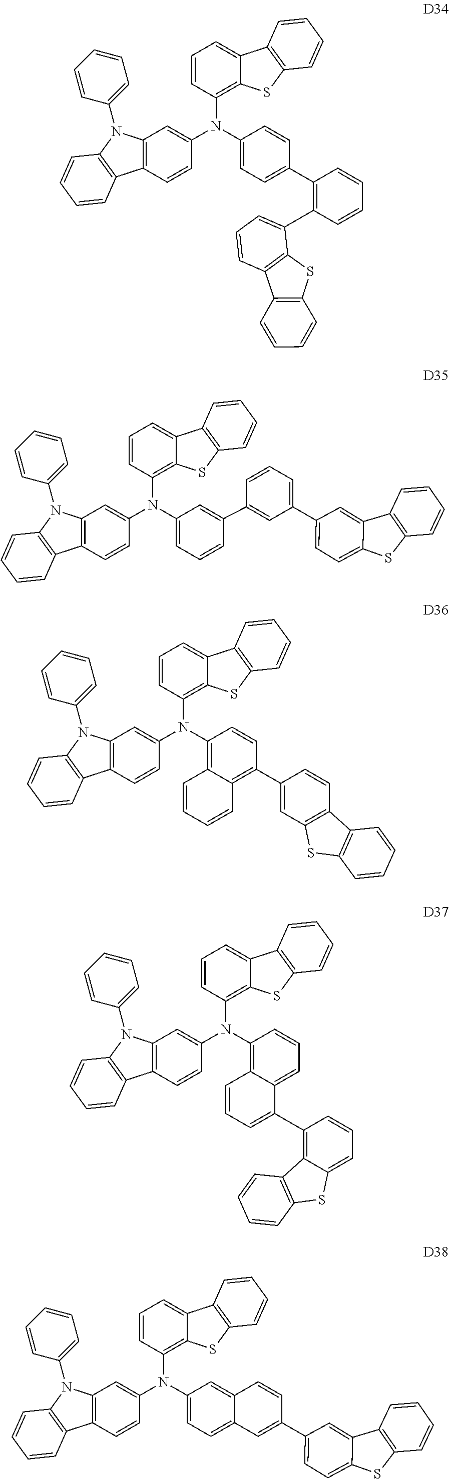 [SMILES: c1ccc(-n2c3ccccc3c3ccc(N(c4ccc(-c5ccccc5-c5cccc6c5sc5ccccc56)cc4)c4cccc5c4sc4ccccc45)cc32)cc1 ; c1ccc(-n2c3ccccc3c3ccc(N(c4cccc(-c5cccc(-c6ccc7sc8ccccc8c7c6)c5)c4)c4cccc5c4sc4ccccc45)cc32)cc1 ; c1ccc(-n2c3ccccc3c3ccc(N(c4ccc(-c5ccc6c(c5)sc5ccccc56)c5ccccc45)c4cccc5c4sc4ccccc45)cc32)cc1 ; c1ccc(-n2c3ccccc3c3ccc(N(c4cccc5c(-c6cccc7sc8ccccc8c67)cccc45)c4cccc5c4sc4ccccc45)cc32)cc1 ; c1ccc(-n2c3ccccc3c3ccc(N(c4ccc5cc(-c6ccc7sc8ccccc8c7c6)ccc5c4)c4cccc5c4sc4ccccc45)cc32)cc1] 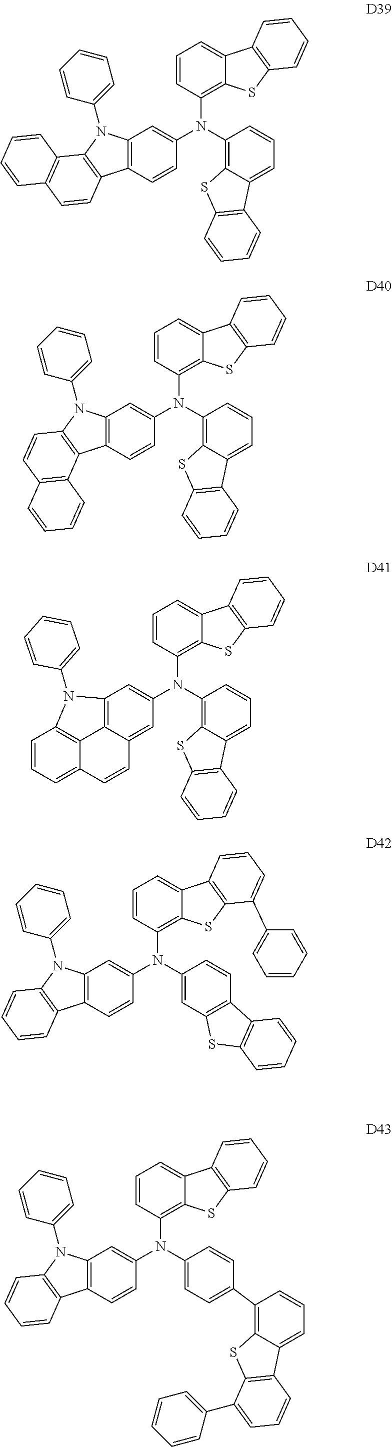 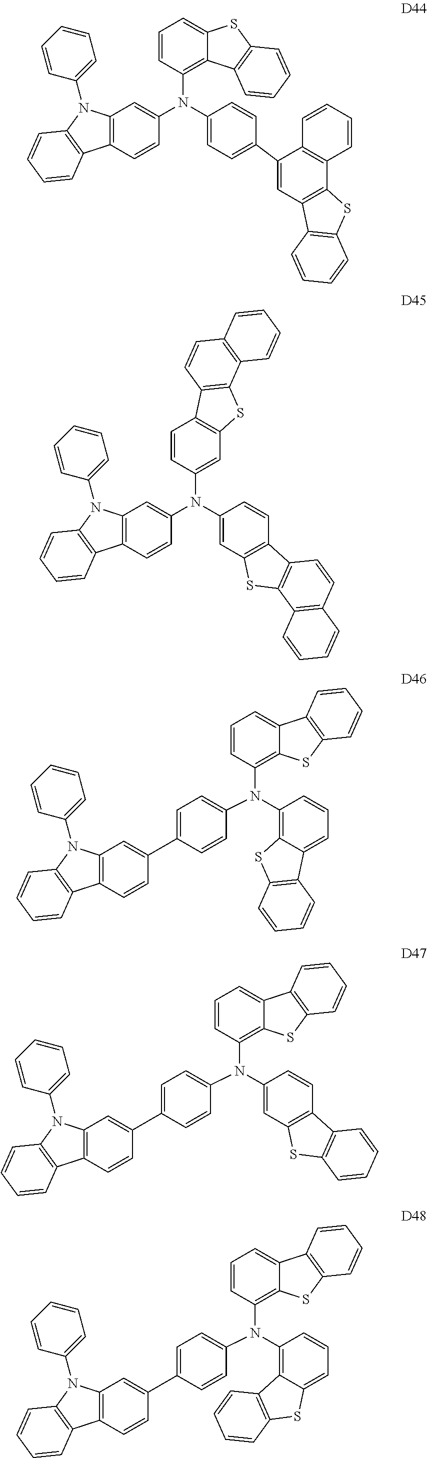 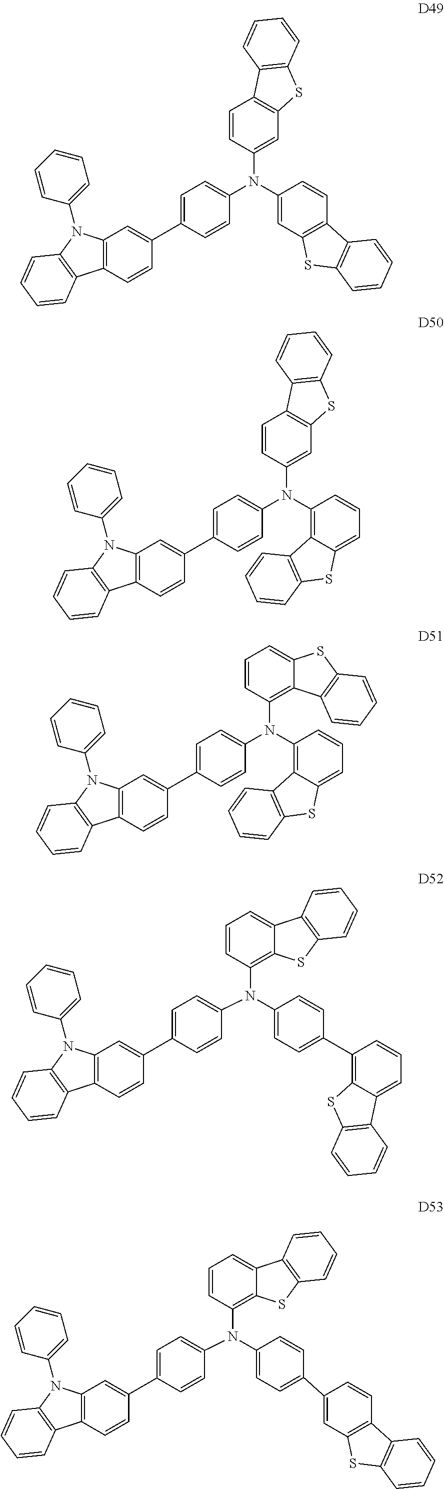 [SMILES: c1ccc(-n2c3ccccc3c3ccc(-c4ccc(N(c5ccc6c(c5)sc5ccccc56)c5ccc6c(c5)sc5ccccc56)cc4)cc32)cc1 ; c1ccc(-n2c3ccccc3c3ccc(-c4ccc(N(c5ccc6c(c5)sc5ccccc56)c5cccc6sc7ccccc7c56)cc4)cc32)cc1 ; c1ccc(-n2c3ccccc3c3ccc(-c4ccc(N(c5cccc6sc7ccccc7c56)c5cccc6sc7ccccc7c56)cc4)cc32)cc1 ; c1ccc(-n2c3ccccc3c3ccc(-c4ccc(N(c5ccc(-c6cccc7c6sc6ccccc67)cc5)c5cccc6c5sc5ccccc56)cc4)cc32)cc1 ; c1ccc(-n2c3ccccc3c3ccc(-c4ccc(N(c5ccc(-c6ccc7c(c6)sc6ccccc67)cc5)c5cccc6c5sc5ccccc56)cc4)cc32)cc1] 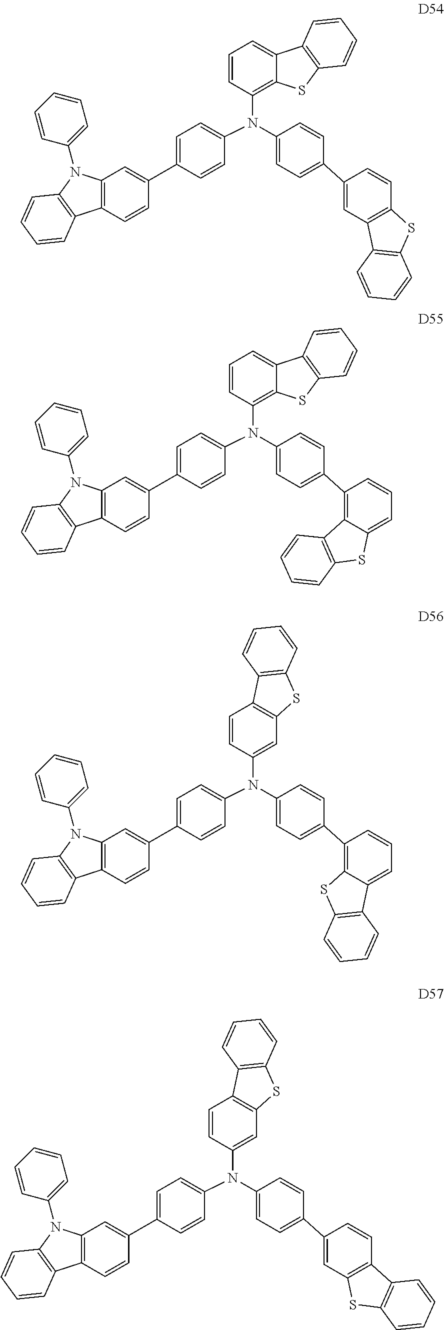 [SMILES: c1ccc(-n2c3ccccc3c3ccc(-c4ccc(N(c5ccc(-c6ccc7sc8ccccc8c7c6)cc5)c5cccc6c5sc5ccccc56)cc4)cc32)cc1 ; c1ccc(-n2c3ccccc3c3ccc(-c4ccc(N(c5ccc(-c6cccc7sc8ccccc8c67)cc5)c5cccc6c5sc5ccccc56)cc4)cc32)cc1 ; c1ccc(-n2c3ccccc3c3ccc(-c4ccc(N(c5ccc(-c6cccc7c6sc6ccccc67)cc5)c5ccc6c(c5)sc5ccccc56)cc4)cc32)cc1 ; c1ccc(-n2c3ccccc3c3ccc(-c4ccc(N(c5ccc(-c6ccc7c(c6)sc6ccccc67)cc5)c5ccc6c(c5)sc5ccccc56)cc4)cc32)cc1] 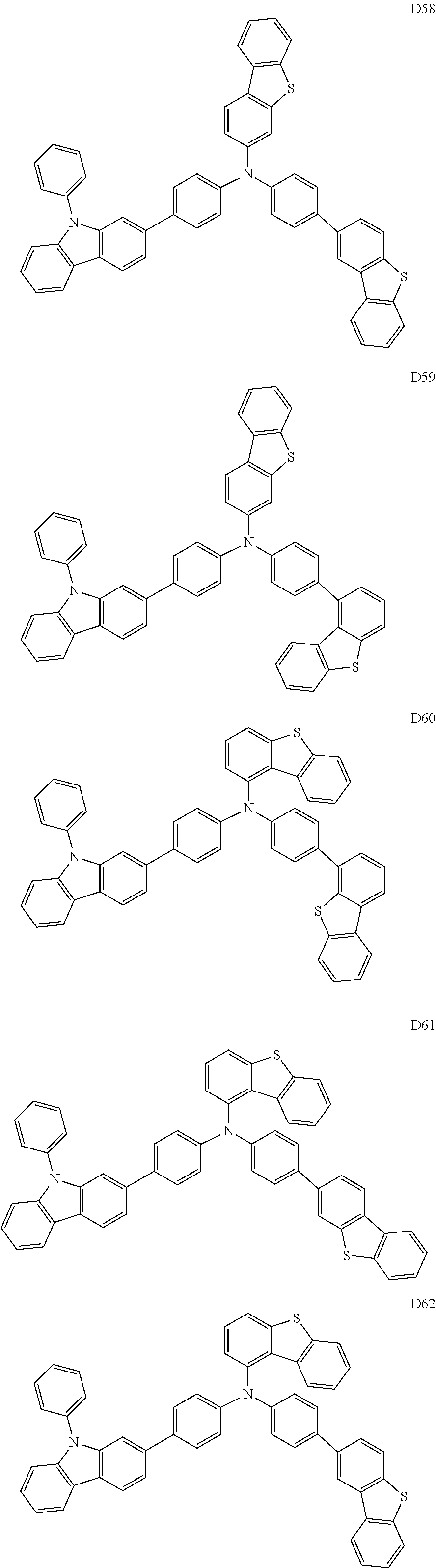 [SMILES: c1ccc(-n2c3ccccc3c3ccc(-c4ccc(N(c5ccc(-c6ccc7sc8ccccc8c7c6)cc5)c5ccc6c(c5)sc5ccccc56)cc4)cc32)cc1 ; c1ccc(-n2c3ccccc3c3ccc(-c4ccc(N(c5ccc(-c6cccc7sc8ccccc8c67)cc5)c5ccc6c(c5)sc5ccccc56)cc4)cc32)cc1 ; c1ccc(-n2c3ccccc3c3ccc(-c4ccc(N(c5ccc(-c6cccc7c6sc6ccccc67)cc5)c5cccc6sc7ccccc7c56)cc4)cc32)cc1 ; c1ccc(-n2c3ccccc3c3ccc(-c4ccc(N(c5ccc(-c6ccc7c(c6)sc6ccccc67)cc5)c5cccc6sc7ccccc7c56)cc4)cc32)cc1 ; c1ccc(-n2c3ccccc3c3ccc(-c4ccc(N(c5ccc(-c6ccc7sc8ccccc8c7c6)cc5)c5cccc6sc7ccccc7c56)cc4)cc32)cc1] 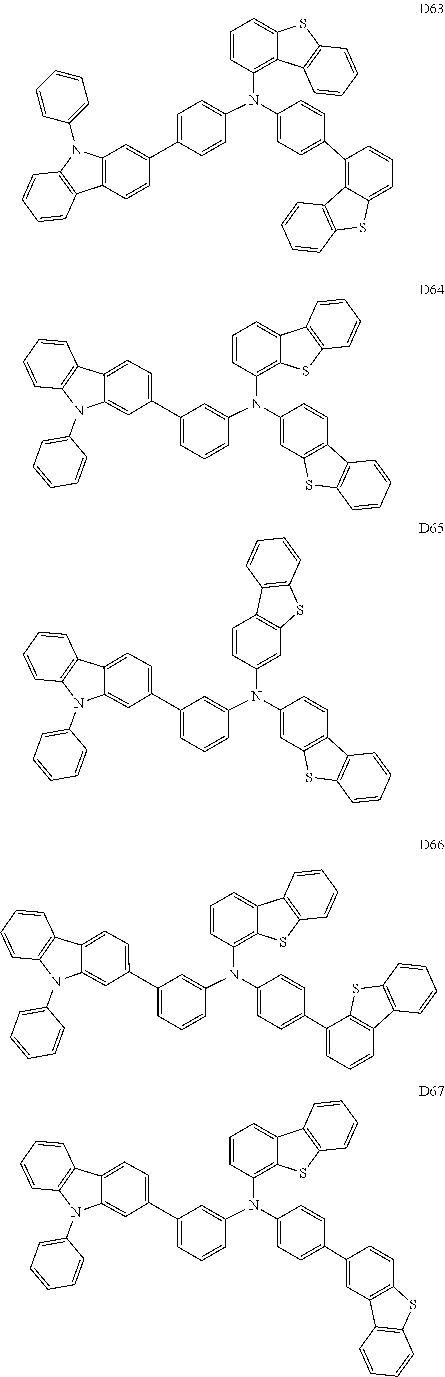 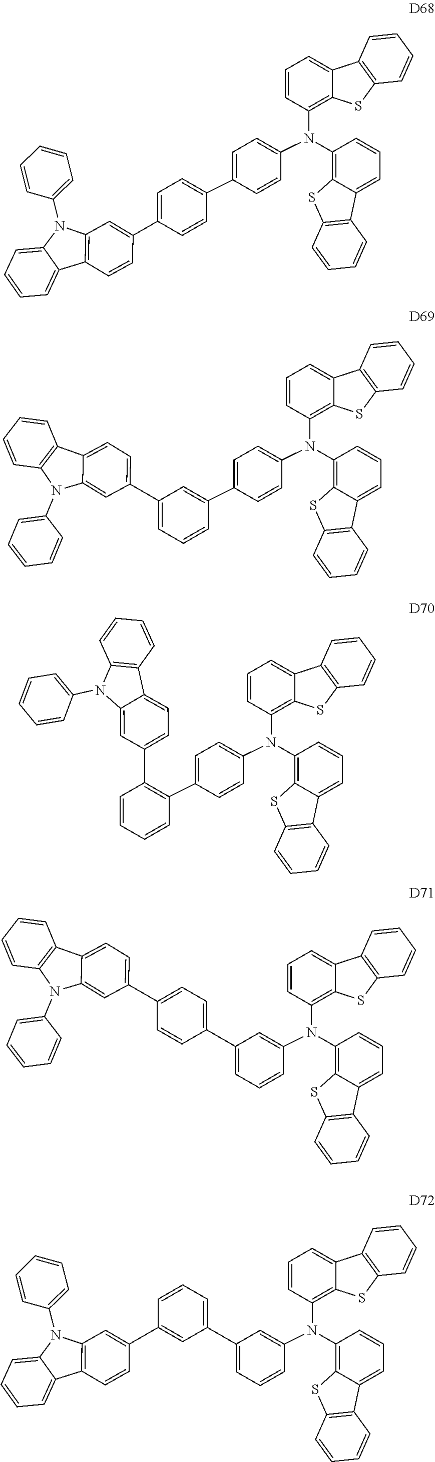 [SMILES: c1ccc(-n2c3ccccc3c3ccc(-c4ccc(-c5ccc(N(c6cccc7c6sc6ccccc67)c6cccc7c6sc6ccccc67)cc5)cc4)cc32)cc1 ; c1ccc(-n2c3ccccc3c3ccc(-c4cccc(-c5ccc(N(c6cccc7c6sc6ccccc67)c6cccc7c6sc6ccccc67)cc5)c4)cc32)cc1 ; c1ccc(-n2c3ccccc3c3ccc(-c4ccccc4-c4ccc(N(c5cccc6c5sc5ccccc56)c5cccc6c5sc5ccccc56)cc4)cc32)cc1 ; c1ccc(-n2c3ccccc3c3ccc(-c4ccc(-c5cccc(N(c6cccc7c6sc6ccccc67)c6cccc7c6sc6ccccc67)c5)cc4)cc32)cc1 ; c1ccc(-n2c3ccccc3c3ccc(-c4cccc(-c5cccc(N(c6cccc7c6sc6ccccc67)c6cccc7c6sc6ccccc67)c5)c4)cc32)cc1] 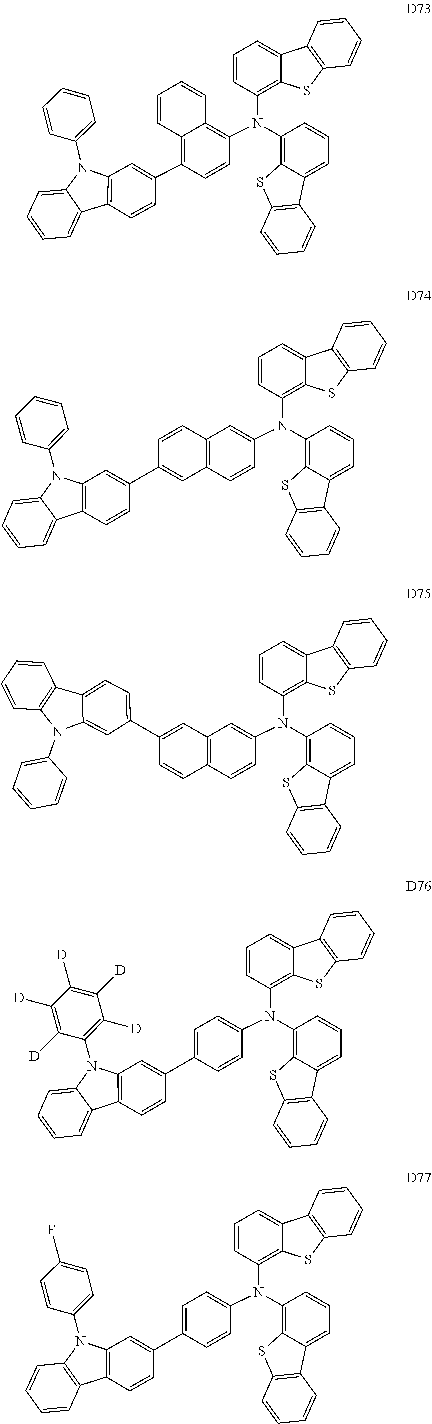 [SMILES: c1ccc(-n2c3ccccc3c3ccc(-c4ccc(N(c5cccc6c5sc5ccccc56)c5cccc6c5sc5ccccc56)c5ccccc45)cc32)cc1 ; c1ccc(-n2c3ccccc3c3ccc(-c4ccc5cc(N(c6cccc7c6sc6ccccc67)c6cccc7c6sc6ccccc67)ccc5c4)cc32)cc1 ; c1ccc(-n2c3ccccc3c3ccc(-c4ccc5ccc(N(c6cccc7c6sc6ccccc67)c6cccc7c6sc6ccccc67)cc5c4)cc32)cc1 ; [2H]c1c([2H])c([2H])c(-n2c3ccccc3c3ccc(-c4ccc(N(c5cccc6c5sc5ccccc56)c5cccc6c5sc5ccccc56)cc4)cc32)c([2H])c1[2H] ; Fc1ccc(-n2c3ccccc3c3ccc(-c4ccc(N(c5cccc6c5sc5ccccc56)c5cccc6c5sc5ccccc56)cc4)cc32)cc1] 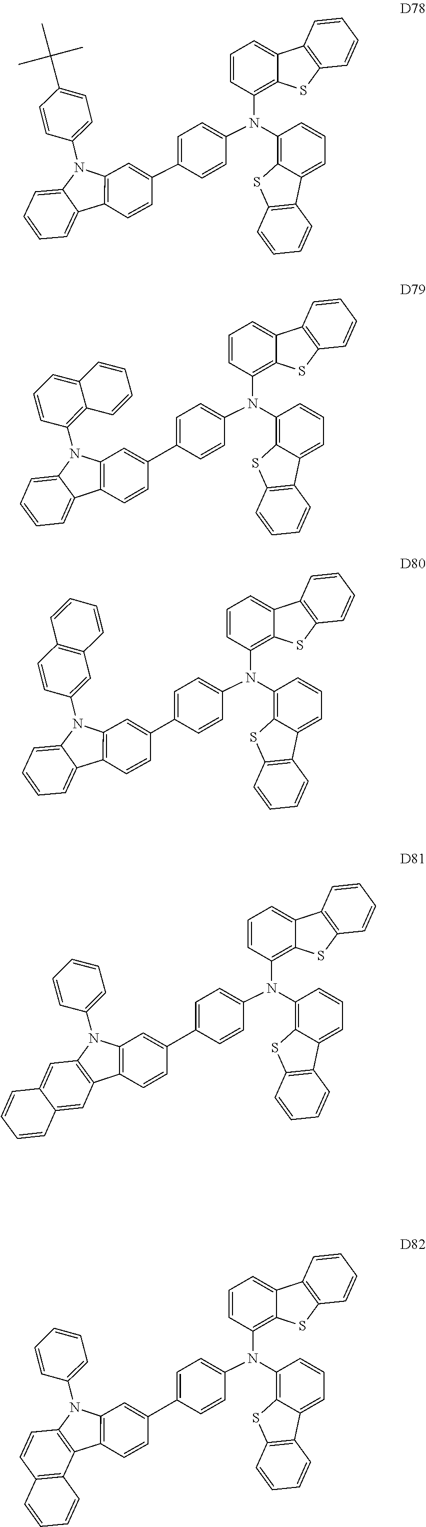 [SMILES: CC(C)(C)c1ccc(-n2c3ccccc3c3ccc(-c4ccc(N(c5cccc6c5sc5ccccc56)c5cccc6c5sc5ccccc56)cc4)cc32)cc1 ; c1ccc2c(-n3c4ccccc4c4ccc(-c5ccc(N(c6cccc7c6sc6ccccc67)c6cccc7c6sc6ccccc67)cc5)cc43)cccc2c1 ; c1ccc2cc(-n3c4ccccc4c4ccc(-c5ccc(N(c6cccc7c6sc6ccccc67)c6cccc7c6sc6ccccc67)cc5)cc43)ccc2c1 ; c1ccc(-n2c3cc(-c4ccc(N(c5cccc6c5sc5ccccc56)c5cccc6c5sc5ccccc56)cc4)ccc3c3cc4ccccc4cc32)cc1 ; c1ccc(-n2c3cc(-c4ccc(N(c5cccc6c5sc5ccccc56)c5cccc6c5sc5ccccc56)cc4)ccc3c3c4ccccc4ccc32)cc1] 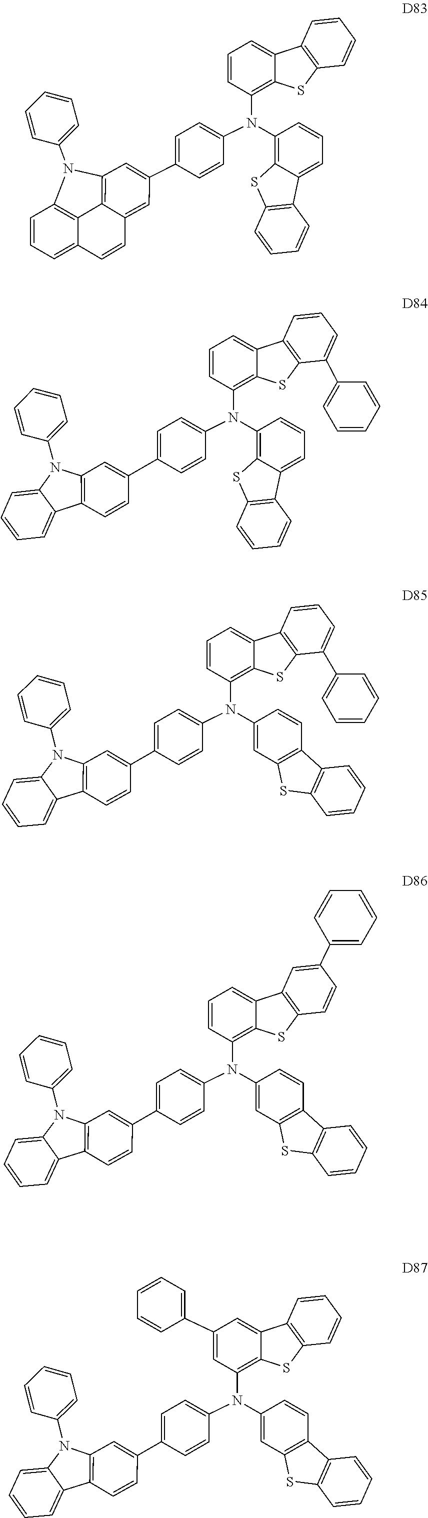 [SMILES: c1ccc(-n2c3cccc4ccc5cc(-c6ccc(N(c7cccc8c7sc7ccccc78)c7cccc8c7sc7ccccc78)cc6)cc2c5c43)cc1 ; c1ccc(-c2cccc3c2sc2c(N(c4ccc(-c5ccc6c7ccccc7n(-c7ccccc7)c6c5)cc4)c4cccc5c4sc4ccccc45)cccc23)cc1 ; c1ccc(-c2cccc3c2sc2c(N(c4ccc(-c5ccc6c7ccccc7n(-c7ccccc7)c6c5)cc4)c4ccc5c(c4)sc4ccccc45)cccc23)cc1 ; c1ccc(-c2ccc3sc4c(N(c5ccc(-c6ccc7c8ccccc8n(-c8ccccc8)c7c6)cc5)c5ccc6c(c5)sc5ccccc56)cccc4c3c2)cc1 ; c1ccc(-c2cc(N(c3ccc(-c4ccc5c6ccccc6n(-c6ccccc6)c5c4)cc3)c3ccc4c(c3)sc3ccccc34)c3sc4ccccc4c3c2)cc1] 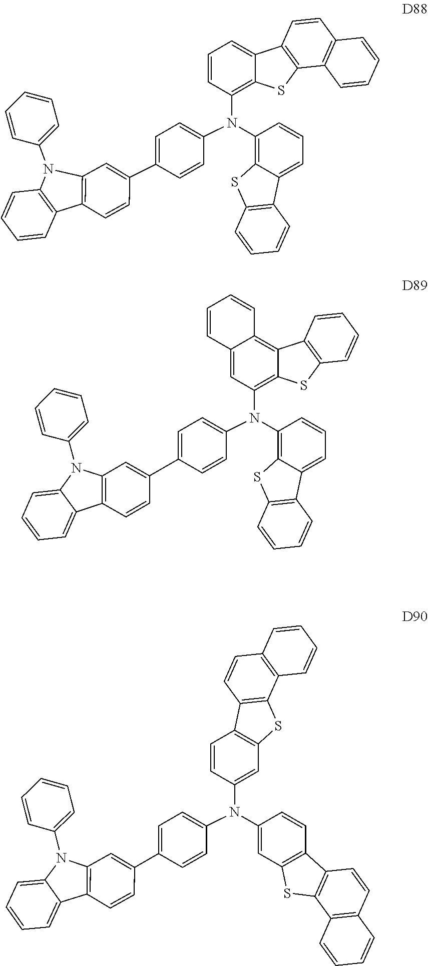 [SMILES: c1ccc(-n2c3ccccc3c3ccc(-c4ccc(N(c5cccc6c5sc5ccccc56)c5cccc6c5sc5c7ccccc7ccc65)cc4)cc32)cc1 ; c1ccc(-n2c3ccccc3c3ccc(-c4ccc(N(c5cccc6c5sc5ccccc56)c5cc6ccccc6c6c5sc5ccccc56)cc4)cc32)cc1 ; c1ccc(-n2c3ccccc3c3ccc(-c4ccc(N(c5ccc6c(c5)sc5c7ccccc7ccc65)c5ccc6c(c5)sc5c7ccccc7ccc65)cc4)cc32)cc1]
